SEQUENCE LISTING (571 sequences)

SEQ ID NO: 1 - DNA, 363 bp, synthetic construct
  source 1..363: mol_type = other DNA
gaggtgcagctggtggagtctgggggaggcttggtacagcctggggggtccctgagactctcctgtgcagcctctggattcgcctttagcagctatgccatgacctgggtccgacaggctccagggaaggggctggagtgggtctcagttatcagtggtactggtggtagtacatactacgcagactccgtgaagggccggttcaccatctccagagacaattccaagaacacgctgtatctacaaatgaacagcctgagagccgaggacacggccgtatattactgtgcgaaagggggagcagctcgtagaatggaatacttccagtactggggccagggcaccctggtcaccgtctcctca

SEQ ID NO: 2 - protein, 121 aa, synthetic construct
EVQLVESGGGLVQPGGSLRLSCAASGFAFSSYAMTWVRQAPGKGLEWVSVISGTGGSTYYADSVKGRFTISRDNSKNTLYLQMNSLRAEDTAVYYCAKGGAARRMEYFQYWGQGTLVTVSS

SEQ ID NO: 3 - protein, 8 aa, synthetic construct
GFAFSSYA

SEQ ID NO: 4 - protein, 8 aa, synthetic construct
ISGTGGST

SEQ ID NO: 5 - protein, 14 aa, synthetic construct
AKGGAARRMEYFQY

SEQ ID NO: 6 - DNA, 321 bp, synthetic construct
  source 1..321: mol_type = other DNA
gacatccagatgacccagtctccatcctccctgtctgcatctgtaggagacagagtcaccatcacttgccgggcgagtcagggcattagcaattatttagcctggtatcagcagaaaccagggaaagttcctaacctccttatctatgctgcatccactttgcaatcaggggtcccatctcgattcagtggcagtggatctgggacagatttcactctcaccatcagcagcctgcagcctgaagatgttgcaacttattactgtcaaaagtataacagtgcccctctcactttcggcggagggaccaaggtggagatcaaa

SEQ ID NO: 7 - protein, 107 aa, synthetic construct
DIQMTQSPSSLSASVGDRVTITCRASQGISNYLAWYQQKPGKVPNLLIYAASTLQSGVPSRFSGSGSGTDFTLTISSLQPEDVATYYCQKYNSAPLTFGGGTKVEIK

SEQ ID NO: 8 - protein, 6 aa, synthetic construct
QGISNY

SEQ ID NO: 9 - skipped

SEQ ID NO: 10 - protein, 9 aa, synthetic construct
QKYNSAPLT

SEQ ID NO: 11 - DNA, 363 bp, synthetic construct
  source 1..363: mol_type = other DNA
gaggtgcagctggtggagtctgggggaggcttggtacagcctggggggtccctgagactctcctgtgcagcctctggattcacctttaacagctatgccatgacctgggtccgccaggctccagggaaggggctggagtgggtctcatttattggtggtagtactggtaacacatactacgcaggctccgtgaagggccggttcaccatctccagcgacaattccaagaagacgctgtatctgcaaatgaacagcctgagagccgaggacacggccgtatattactgtgcgaaagggggagcagctcgtagaatggaatacttccagcactggggccagggcaccctggtcaccgtctcctca

SEQ ID NO: 12 - protein, 121 aa, synthetic construct
EVQLVESGGGLVQPGGSLRLSCAASGFTFNSYAMTWVRQAPGKGLEWVSFIGGSTGNTYYAGSVKGRFTISSDNSKKTLYLQMNSLRAEDTAVYYCAKGGAARRMEYFQHWGQGTLVTVSS

SEQ ID NO: 13 - protein, 8 aa, synthetic construct
GFTFNSYA

SEQ ID NO: 14 - protein, 8 aa, synthetic construct
IGGSTGNT

SEQ ID NO: 15 - protein, 14 aa, synthetic construct
AKGGAARRMEYFQH

SEQ ID NO: 16 - DNA, 321 bp, synthetic construct
  source 1..321: mol_type = other DNA
gacatccagatgacccagtctccatcctccctgtctgcatctataggagacagagtcaccatcacttgccgggcgagtcagggcattagcaattatttagcctggtatcaacagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaaccataacagtgtccctctcactttcggcggagggaccaaggtggagatcaaa

SEQ ID NO: 17 - protein, 107 aa, synthetic construct
DIQMTQSPSSLSASIGDRVTITCRASQGISNYLAWYQQKPGKVPKLLIYAASTLQSGVPSRFSGSGSGTDFTLTISSLQPEDVATYYCQNHNSVPLTFGGGTKVEIK

SEQ ID NO: 18 - protein, 6 aa, synthetic construct
QGISNY

SEQ ID NO: 19 - skipped

SEQ ID NO: 20 - protein, 9 aa, synthetic construct
QNHNSVPLT

SEQ ID NO: 21 - DNA, 366 bp, synthetic construct
  source 1..366: mol_type = other DNA
caggtgcagctggtggagtctgggggaggcgtggtccagcctgggaggtccctgagactctcctgtgcagcgtctggattcaccttcactacctatggcatgcactgggtccgccaggctccaggcaaggggctggagtgggtggctgttatatggtatgatggaagtaataaatattatggagactccgtgaagggccgattcaccatctccagagacaattccaagaacacactgtatctgcaaatgaacagcctgagagtcgacgacacggctgtttattactgtacgagaacccatggctataccaggtcgtcggacggttttgactactggggccagggaaccctggtcaccgtctcctca

SEQ ID NO: 22 - protein, 122 aa, synthetic construct
QVQLVESGGGVVQPGRSLRLSCAASGFTFTTYGMHWVRQAPGKGLEWVAVIWYDGSNKYYGDSVKGRFTISRDNSKNTLYLQMNSLRVDDTAVYYCTRTHGYTRSSDGFDYWGQGTLVTVSS

SEQ ID NO: 23 - protein, 8 aa, synthetic construct
GFTFTTYG

SEQ ID NO: 24 - protein, 8 aa, synthetic construct
IWYDGSNK

SEQ ID NO: 25 - protein, 15 aa, synthetic construct
TRTHGYTRSSDGFDY

SEQ ID NO: 26 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattagaaatgttttaggctggtttcagcagaaaccagggaaagcccctcagcgcctgatctatgctgcatccagtttgcaaagtggggtcccatcaaggttcagcggcagtggatctgggacagaattcactctcacaatcagcagcctacagcctgaagattttgcaacttattactgtctacagcataatttttacccgctcactttcggcggagggaccaaggtggagatcaaa

SEQ ID NO: 27 - protein, 107 aa, synthetic construct
DIQMTQSPSSLSASVGDRVTITCRASQSIRNVLGWFQQKPGKAPQRLIYAASSLQSGVPSRFSGSGSGTEFTLTISSLQPEDFATYYCLQHNFYPLTFGGGTKVEIK

SEQ ID NO: 28 - protein, 6 aa, synthetic construct
QSIRNV

SEQ ID NO: 29 - skipped

SEQ ID NO: 30 - protein, 9 aa, synthetic construct
LQHNFYPLT

SEQ ID NO: 31 - DNA, 369 bp, synthetic construct
  source 1..369: mol_type = other DNA
gaagtgcagctggtggagtctgggggaggcttggtacagcctggcaggtccctgagactctcctgtgcagcctctggattcacctttgatgataaagccatgcactgggtccggcaagttccagggaagggcctggaatggatctcaggtattagttggaatagtggtactataggctatgcggactctgtgaagggccgattcatcatctccagagacaacgccaagaactccctgtatctacaaatgaacagtctgagagctgaggacacggccttgtattactgcgcaaaagatggagataccagtggctggtactggtacggtttggacgtctggggccaagggaccacggtcaccgtctcctca

SEQ ID NO: 32 - protein, 123 aa, synthetic construct
EVQLVESGGGLVQPGRSLRLSCAASGFTFDDKAMHWVRQVPGKGLEWISGISWNSGTIGYADSVKGRFIISRDNAKNSLYLQMNSLRAEDTALYYCAKDGDTSGWYWYGLDVWGQGTTVTVSS

SEQ ID NO: 33 - protein, 8 aa, synthetic construct
GFTFDDKA

SEQ ID NO: 34 - protein, 8 aa, synthetic construct
ISWNSGTI

SEQ ID NO: 35 - protein, 16 aa, synthetic construct
AKDGDTSGWYWYGLDV

SEQ ID NO: 36 - DNA, 321 bp, synthetic construct
  source 1..321: mol_type = other DNA
gaaattgtgttgacacagtctcctgccaccctgtctttgtctccaggggaaagagccaccctctcctgcagggccagtcagagtgttagcagctacttagcctggtaccaacagaaacctggccaggctcccaggctcctcatccatgatgtatccaacagggccactggcatcccagccaggttcagtggcagtgggtctgggacagacttcactctcaccatcagcagtctagagcctgaagattttgtagtttattactgtcagcagcgtagcgactggcccatcaccttcggccaagggacacgactggagattaaa

SEQ ID NO: 37 - protein, 107 aa, synthetic construct
EIVLTQSPATLSLSPGERATLSCRASQSVSSYLAWYQQKPGQAPRLLIHDVSNRATGIPARFSGSGSGTDFTLTISSLEPEDFVVYYCQQRSDWPITFGQGTRLEIK

SEQ ID NO: 38 - protein, 6 aa, synthetic construct
QSVSSY

SEQ ID NO: 39 - skipped

SEQ ID NO: 40 - protein, 9 aa, synthetic construct
QQRSDWPIT

SEQ ID NO: 41 - DNA, 360 bp, synthetic construct
  source 1..360: mol_type = other DNA
caggtgcagctggtggagtctgggggaggcgtggtccagcctgggaggtccctgagactctcctgtatagcctctggattcaccttcagtgtctatggcattcactgggtccgccaggctccaggcaaggggctggagtggatggcagtaatatcacatgatggaaatattaaacactatgcagactccgtgaagggccgattcaccatctccagagacaattccaagaacacgctgtatcttcaaattaacagcctgagaactgaggacacggctgtgtattactgtgcgaaagatacctggaactcccttgatacttttgatatctggggccaagggacaatggtcaccgtctcttca

SEQ ID NO: 42 - protein, 120 aa, synthetic construct
QVQLVESGGGVVQPGRSLRLSCIASGFTFSVYGIHWVRQAPGKGLEWMAVISHDGNIKHYADSVKGRFTISRDNSKNTLYLQINSLRTEDTAVYYCAKDTWNSLDTFDIWGQGTMVTVSS

SEQ ID NO: 43 - protein, 8 aa, synthetic construct
GFTFSVYG

SEQ ID NO: 44 - protein, 8 aa, synthetic construct
ISHDGNIK

SEQ ID NO: 45 - protein, 13 aa, synthetic construct
AKDTWNSLDTFDI

SEQ ID NO: 46 - DNA, 321 bp, synthetic construct
  source 1..321: mol_type = other DNA
gacatccagttgacccagtctccatcctccctgtctgcatctgtaggagacagagtcaccatcacttgctgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgtcaacagcttaatagttaccctctcactttcggcggagggaccaaggtggagatcaaa

SEQ ID NO: 47 - protein, 107 aa, synthetic construct
DIQLTQSPSSLSASVGDRVTITCWASQGISSYLAWYQQKPGKAPKLLIYAASTLQSGVPSRFSGSGSGTEFTLTISSLQPEDFATYYCQQLNSYPLTFGGGTKVEIK

SEQ ID NO: 48 - protein, 6 aa, synthetic construct
QGISSY

SEQ ID NO: 49 - skipped

SEQ ID NO: 50 - protein, 9 aa, synthetic construct
QQLNSYPLT

SEQ ID NO: 51 - DNA, 360 bp, synthetic construct
  source 1..360: mol_type = other DNA
caggtcaccttgagggagtctggtcccgcgctggtgaaaccctcacagaccctcacactgacctgcaccttctctggattctcactcaacacttatgggatgtttgtgagctggatccgtcagcctccagggaaggccctagagtggcttgcacacattcattgggatgatgataaatactacagcacatctctgaagaccaggctcaccatctccaaggacacctccaaaaaccaggtggtccttacaatgaccaacatggaccctgtggacacagccacgtattattgtgcacgggggcacaataatttgaactacatcatccactggggccagggaaccctggtcaccgtctcctca

SEQ ID NO: 52 - protein, 120 aa, synthetic construct
QVTLRESGPALVKPSQTLTLTCTFSGFSLNTYGMFVSWIRQPPGKALEWLAHIHWDDDKYYSTSLKTRLTISKDTSKNQVVLTMTNMDPVDTATYYCARGHNNLNYIIHWGQGTLVTVSS

SEQ ID NO: 53 - protein, 10 aa, synthetic construct
GFSLNTYGMF

SEQ ID NO: 54 - protein, 7 aa, synthetic construct
IHWDDDK

SEQ ID NO: 55 - protein, 12 aa, synthetic construct
ARGHNNLNYIIH

SEQ ID NO: 56 - DNA, 321 bp, synthetic construct
  source 1..321: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaagctcctgatctatgctgcatccactttacaaagtggggtcccatcaaggttcagcggcagtggatctggcacagatttcactctcaccatcagcagcctgcagcctgaagattttgcaacttattactgtctacaagattacaattacccattcactttcggccctgggaccaaagtggatatcaaa

SEQ ID NO: 57 - protein, 107 aa, synthetic construct
AIQMTQSPSSLSASVGDRVTITCRASQGIRNDLGWYQQKPGKAPKLLIYAASTLQSGVPSRFSGSGSGTDFTLTISSLQPEDFATYYCLQDYNYPFTFGPGTKVDIK

SEQ ID NO: 58 - protein, 6 aa, synthetic construct
QGIRND

SEQ ID NO: 59 - skipped

SEQ ID NO: 60 - protein, 9 aa, synthetic construct
LQDYNYPFT

SEQ ID NO: 61 - DNA, 348 bp, synthetic construct
  source 1..348: mol_type = other DNA
gaggtgcagctggtggagtctgggggaggcttggtacagcctggagggtccctgagactctcctgtgcagtctctggattcatcttcagtagttatgaaatgaactgggtccgccaggctccagggaaggggctggagtgggtttcatacattagtagtagtggtagtaccatattctacgcagactctgtgaagggccgattcaccatctccagagacaacgccaagaactcactgtatctgcaaatgaacagcctgagagccgaggacacggctgtttattactgtgtgtctggagtggtcctttttgatgtctggggccaagggacaatggtcaccgtctcttca

SEQ ID NO: 62 - protein, 116 aa, synthetic construct
EVQLVESGGGLVQPGGSLRLSCAVSGFIFSSYEMNWVRQAPGKGLEWVSYISSSGSTIFYADSVKGRFTISRDNAKNSLYLQMNSLRAEDTAVYYCVSGVVLFDVWGQGTMVTVSS

SEQ ID NO: 63 - protein, 8 aa, synthetic construct
GFIFSSYE

SEQ ID NO: 64 - protein, 8 aa, synthetic construct
ISSSGSTI

SEQ ID NO: 65 - protein, 9 aa, synthetic construct
VSGVVLFDV

SEQ ID NO: 66 - DNA, 321 bp, synthetic construct
  source 1..321: mol_type = other DNA
gaaatagtgatgacgcagtctccagccaccctgtctgtgtctccgggggaaagagccaccctctcctgcagggccagtcagagtgttagcagcaactttgcctggtaccaacagaaacctggccaggctcccaggctcctcatctatagtgcatcctccagggccactggtatcccagtcaggttcagtggcagtgggtctgggacagagttcactctcaccatcagcagcctgcagtctgaagattttgcagtttattactgtcagcagtataatatctggcctcggacgttcggccaagggaccaaggtggaaatcaaa

SEQ ID NO: 67 - protein, 107 aa, synthetic construct
EIVMTQSPATLSVSPGERATLSCRASQSVSSNFAWYQQKPGQAPRLLIYSASSRATGIPVRFSGSGSGTEFTLTISSLQSEDFAVYYCQQYNIWPRTFGQGTKVEIK

SEQ ID NO: 68 - protein, 6 aa, synthetic construct
QSVSSN

SEQ ID NO: 69 - skipped

SEQ ID NO: 70 - protein, 9 aa, synthetic construct
QQYNIWPRT

SEQ ID NO: 71 - DNA, 375 bp, synthetic construct
  source 1..375: mol_type = other DNA
gaggtgcagctggtggagtctgggggaggcttggtccagcctggggggtccctgagactctcctgtgcagcctctggattcacctttagtaactattggatgacctgggtccgccaggctccagggaaggggctggagtgggtggccaacataaaggaagatggaagtgagaaagactatgtggactctgtgaagggccgattcaccatctccagagacaacgccaagaactcactgtatctgcaaatgaacagcctgagaggcgaggacacggctgtgtattactgtgcgagagatggggagcagctcgtcgattactactactactacgttatggacgtctggggccaagggaccacggtcaccgtctcctca

SEQ ID NO: 72 - protein, 125 aa, synthetic construct
EVQLVESGGGLVQPGGSLRLSCAASGFTFSNYWMTWVRQAPGKGLEWVANIKEDGSEKDYVDSVKGRFTISRDNAKNSLYLQMNSLRGEDTAVYYCARDGEQLVDYYYYYVMDVWGQGTTVTVSS

SEQ ID NO: 73 - protein, 8 aa, synthetic construct
GFTFSNYW

SEQ ID NO: 74 - protein, 8 aa, synthetic construct
IKEDGSEK

SEQ ID NO: 75 - protein, 18 aa, synthetic construct
ARDGEQLVDYYYYYVMDV

SEQ ID NO: 76 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaaaaggctaacagtttcccgtacacttttggccaggggaccaagctggagatcaaa

SEQ ID NO: 77 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKANSFPYTFGQGTKLEIK

SEQ ID NO: 78 - protein, 6 aa, synthetic construct
QGISSW

SEQ ID NO: 79 - skipped

SEQ ID NO: 80 - protein, 9 aa, synthetic construct
QKANSFPYT

SEQ ID NO: 81 - DNA, 381 bp, synthetic construct
  source 1..381: mol_type = other DNA
gaagtgcagctggtggagtctgggggaggcttggtacagcctggcaggtccctgagactctcctgtgcagcctctggattcacctttgatgactatgccatgcactgggtccggcaagctccagggaagggcctggagtgggtctcaggtattagttggaatagtggttacataggctatgcggactctgtgaagggccgattcaccatctccagagacaacgccgagaactccctacatctgcaaatgaacagtctgagagctgaggacacggccttgtattactgtgcaagagggggatctactctggttcggggagttaagggaggctactacggtatggacgtctggggccaagggaccacggtcaccgtctcctca

SEQ ID NO: 82 - protein, 127 aa, synthetic construct
EVQLVESGGGLVQPGRSLRLSCAASGFTFDDYAMHWVRQAPGKGLEWVSGISWNSGYIGYADSVKGRFTISRDNAENSLHLQMNSLRAEDTALYYCARGGSTLVRGVKGGYYGMDVWGQGTTVTVSS

SEQ ID NO: 83 - protein, 8 aa, synthetic construct
GFTFDDYA

SEQ ID NO: 84 - protein, 8 aa, synthetic construct
ISWNSGYI

SEQ ID NO: 85 - protein, 20 aa, synthetic construct
ARGGSTLVRGVKGGYYGMDV

SEQ ID NO: 86 - DNA, 321 bp, synthetic construct
  source 1..321: mol_type = other DNA
gacatccagatgacccagtctccatcctccctgtctgcatctgtaggagacagagtcaccatcacttgccgggcaagtcagagcataagtagctatttaaattggtatcagcagaaaccaggtaaagcccctaaggtcctgatctatgctgcatccagtttgcaaagtggggtcccatcaaggttcagtggcagtggatctgggacagatttcactctcaccatcagcagtctgcaacctgaagattttgcaacttactactgtcaacagagttacagtattccgctcactttcggcggagggaccaaggtggagatcaaa

SEQ ID NO: 87 - protein, 107 aa, synthetic construct
DIQMTQSPSSLSASVGDRVTITCRASQSISSYLNWYQQKPGKAPKVLIYAASSLQSGVPSRFSGSGSGTDFTLTISSLQPEDFATYYCQQSYSIPLTFGGGTKVEIK

SEQ ID NO: 88 - protein, 6 aa, synthetic construct
QSISSY

SEQ ID NO: 89 - skipped

SEQ ID NO: 90 - protein, 9 aa, synthetic construct
QQSYSIPLT

SEQ ID NO: 91 - DNA, 351 bp, synthetic construct
  source 1..351: mol_type = other DNA
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacatttccaagaacacgctgtatctgcaaatgaacagcctgagagccgaggacacggccgtatattactgtgcgggtcaactggatctcttctttgactactggggccagggaaccctggtcaccgtctcctca

SEQ ID NO: 92 - protein, 117 aa, synthetic construct
QVQLVESGGGVVQPGRSLRLSCAASGFTFSSYGMHWVRQAPGKGLEWVAVIWYDGSNKYYADSVKGRFTISRDISKNTLYLQMNSLRAEDTAVYYCAGQLDLFFDYWGQGTLVTVSS

SEQ ID NO: 93 - protein, 8 aa, synthetic construct
GFTFSSYG

SEQ ID NO: 94 - protein, 8 aa, synthetic construct
IWYDGSNK

SEQ ID NO: 95 - protein, 10 aa, synthetic construct
AGQLDLFFDY

SEQ ID NO: 96 - DNA, 321 bp, synthetic construct
  source 1..321: mol_type = other DNA
gacatccagatgacccagtctccatcctccctgtctgcatctgtaggagacagagtcaccatcacttgccgggcaagtcagagcattgacaggtatttaaattggtatcggcagaaaccagggaaagcccctaagctcctgatctatactacatccagtttgcaaagtggggtcccatcaaggttcagtggcagtggatctgggacagatttcactctcaccctcagcagtctgcagcctgaagattttgcaacttactactgtcagcagagttacagtcccccgctcactttcggcggagggaccaaggtggagatcaaa

SEQ ID NO: 97 - protein, 107 aa, synthetic construct
DIQMTQSPSSLSASVGDRVTITCRASQSIDRYLNWYRQKPGKAPKLLIYTTSSLQSGVPSRFSGSGSGTDFTLTLSSLQPEDFATYYCQQSYSPPLTFGGGTKVEIK

SEQ ID NO: 98 - protein, 6 aa, synthetic construct
QSIDRY

SEQ ID NO: 99 - skipped

SEQ ID NO: 100 - protein, 9 aa, synthetic construct
QQSYSPPLT

SEQ ID NO: 101 - DNA, 375 bp, synthetic construct
  source 1..375: mol_type = other DNA
gaggtgcagctggtggagtctgggggaggcttggtccagcctggggggtccctgagactctcctgtacagcctctggattcacctttagtaactattggatgacctgggtccgccaggctccagggaaggggctggagtgggtggccaacataaaggaagatggaagtgagaaagagtatgtggactctgtgaagggccggttcaccatctccagagacaacgccaagaattcactgtatctgcaaatgaacagcctgagaggcgaggacacggctgtatattactgtgcgagagatggggagcagctcgtcgattactattactactacgttatggacgtctggggccaagggaccacggtcaccgtctcctca

SEQ ID NO: 102 - protein, 125 aa, synthetic construct
EVQLVESGGGLVQPGGSLRLSCTASGFTFSNYWMTWVRQAPGKGLEWVANIKEDGSEKEYVDSVKGRFTISRDNAKNSLYLQMNSLRGEDTAVYYCARDGEQLVDYYYYYVMDVWGQGTTVTVSS

SEQ ID NO: 103 - protein, 8 aa, synthetic construct
GFTFSNYW

SEQ ID NO: 104 - protein, 8 aa, synthetic construct
IKEDGSEK

SEQ ID NO: 105 - protein, 18 aa, synthetic construct
ARDGEQLVDYYYYYVMDV

SEQ ID NO: 106 - DNA, 321 bp, synthetic construct
  source 1..321: mol_type = other DNA
gacatccagatgacccagtctccatcttccgtgtctgcatctgttggagacagagtcaccatcacttgtcgggcgagtcagggtattagcagctggttagcctggtatcagcagaaaccagggaaagcccctaagctcctgatctatgctgcatccagtttgcaaagtggggtcccatcaaggttcagcggcagtggatctgggacagatttcactctcaccatcagcagcctgcagcctgaagattttgcaacttactattgtcaaaaggctgacagtctcccgtacgcttttggccaggggaccaagctggagatcaaa

SEQ ID NO: 107 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKADSLPYAFGQGTKLEIK

SEQ ID NO: 108 - protein, 6 aa, synthetic construct
QGISSW

SEQ ID NO: 109 - skipped

SEQ ID NO: 110 - protein, 9 aa, synthetic construct
QKADSLPYA

SEQ ID NO: 111 - DNA, 363 bp, synthetic construct
  source 1..363: mol_type = other DNA
gaggtgcagctggtggagtctgggggaggcttggttcagcctggggggtccctgagactctcctgtgcaacctctggattcacctttaccagctatgacatgaagtgggtccgccaggctccagggctgggcctggagtgggtctcagctattagtggtagtggtggtaacacatactacgcagactccgtgaagggccggttcaccatctccagagacaattccaggaacacgctgtatctgcaaatgaacagcctgagagccgaggacacggccgtatattactgtacgaggtcccatgacttcggtgccttcgactactttgactactggggccagggaaccctggtcaccgtctcctca

SEQ ID NO: 112 - protein, 121 aa, synthetic construct
EVQLVESGGGLVQPGGSLRLSCATSGFTFTSYDMKWVRQAPGLGLEWVSAISGSGGNTYYADSVKGRFTISRDNSRNTLYLQMNSLRAEDTAVYYCTRSHDFGAFDYFDYWGQGTLVTVSS

SEQ ID NO: 113 - protein, 8 aa, synthetic construct
GFTFTSYD

SEQ ID NO: 114 - protein, 8 aa, synthetic construct
ISGSGGNT

SEQ ID NO: 115 - protein, 14 aa, synthetic construct
TRSHDFGAFDYFDY

SEQ ID NO: 116 - DNA, 321 bp, synthetic construct
  source 1..321: mol_type = other DNA
gacatccagatgacccagtctccatcctccctgtctgcatctgtgggagacagagtcaccatcacttgccgggcaagtcagggcattagagatcattttggctggtatcagcagaaaccagggaaagcccctaagcgcctgatctatgctgcatccagtttgcacagtggggtcccatcaaggttcagcggcagtggatctgggacagaattcactctcacaatcagcagcttgcagcctgaagattttgcaacctattactgtctacagtatgatacttacccgctcactttcggcggagggaccaaggtggagatcaaa

SEQ ID NO: 117 - protein, 107 aa, synthetic construct
DIQMTQSPSSLSASVGDRVTITCRASQGIRDHFGWYQQKPGKAPKRLIYAASSLHSGVPSRFSGSGSGTEFTLTISSLQPEDFATYYCLQYDTYPLTFGGGTKVEIK

SEQ ID NO: 118 - protein, 6 aa, synthetic construct
QGIRDH

SEQ ID NO: 119 - skipped

SEQ ID NO: 120 - protein, 9 aa, synthetic construct
LQYDTYPLT

SEQ ID NO: 121 - DNA, 366 bp, synthetic construct
  source 1..366: mol_type = other DNA
gaagtgcagctggtggagtctgggggagacttggtacagcctggcaggtccctgagactctcctgtgcagcctctggattcacctttgatgattatgccatgcactgggtccggcaagctccagggaagggcctggagtgggtctcaggtattagttggaatagtgctaccagagtctatgcggactctgtgaagggccgattcaccatctccagagacaacgccaagaatttcctgtatctgcaaatgaacagtctgagatctgaggacacggccttgtatcactgtgcaaaagatatggatatctcgctagggtactacggtttggacgtctggggccaagggaccacggtcaccgtctcctca

SEQ ID NO: 122 - protein, 122 aa, synthetic construct
EVQLVESGGDLVQPGRSLRLSCAASGFTFDDYAMHWVRQAPGKGLEWVSGISWNSATRVYADSVKGRFTISRDNAKNFLYLQMNSLRSEDTALYHCAKDMDISLGYYGLDVWGQGTTVTVSS

SEQ ID NO: 123 - protein, 8 aa, synthetic construct
GFTFDDYA

SEQ ID NO: 124 - protein, 8 aa, synthetic construct
ISWNSATR

SEQ ID NO: 125 - protein, 15 aa, synthetic construct
AKDMDISLGYYGLDV

SEQ ID NO: 126 - DNA, 324 bp, synthetic construct
  source 1..324: mol_type = other DNA
gaaatagtgatgacgcagtctccagccaccctgtctgtgtctccaggggaaagagccaccctctcctgcagggccagtcagactgttagcagcaacttagcctggtatcagcagaaacctggccaggctcccaggctcctcatctatggttcatcctccagggccactggtatcccagccaggttcagtggcagtgggtctgggacagagttcactctcaccatcagcagcctgcagtctgaagattttgcagtttattactgtcagcagtataataactggcctccctacacttttggccaggggaccaagctggagatcaaa

SEQ ID NO: 127 - protein, 108 aa, synthetic construct
EIVMTQSPATLSVSPGERATLSCRASQTVSSNLAWYQQKPGQAPRLLIYGSSSRATGIPARFSGSGSGTEFTLTISSLQSEDFAVYYCQQYNNWPPYTFGQGTKLEIK

SEQ ID NO: 128 - protein, 6 aa, synthetic construct
QTVSSN

SEQ ID NO: 129 - skipped

SEQ ID NO: 130 - protein, 10 aa, synthetic construct
QQYNNWPPYT

SEQ ID NO: 131 - DNA, 372 bp, synthetic construct
  source 1..372: mol_type = other DNA
cagatcaccttgaaggagtctggtcctacgctggtgaaacccacacagaccctcacgctgacctgcaccttctctgggttctcactcagcactagtggagtgggtgtggtctggatccgtcagccccccggaaaggccctggagtggcttgcactcatttattggaatgatcataagcggtacagcccatctctggggagcaggctcaccatcaccaaggacacctccaaaaaccaggtggtccttacaatgaccaacatggaccctgtggacacagccacatattactgtgcacactacagtgggagctattcctactactactatggtttggacgtctggggccaagggaccacggtcaccgtctcctca

SEQ ID NO: 132 - protein, 124 aa, synthetic construct
QITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSS

SEQ ID NO: 133 - protein, 10 aa, synthetic construct
GFSLSTSGVG

SEQ ID NO: 134 - protein, 7 aa, synthetic construct
IYWNDHK

SEQ ID NO: 135 - protein, 16 aa, synthetic construct
AHYSGSYSYYYYGLDV

SEQ ID NO: 136 - DNA, 321 bp, synthetic construct
  source 1..321: mol_type = other DNA
gacatccagatgacccagtctccatcttccgtgtctgcatctgtaggagacagagtcaccatcacttgtcgggcgagtcagggtattgccagctggttagcctggtatcagcagaaaccagggaaagcccctgagctcctgatctatgctgcatccagtttgcaaggtggggtcccatcaaggttcagcggcagtggatctgggacagatttcactctcaccatcagcagcctgcagcctgaagattttgcaatttactattgtcaacaggctaactatttcccgtggacgttcggccaagggaccaaggtggaaatcaaa

SEQ ID NO: 137 - protein, 107 aa, synthetic construct
DIQMTQSPSSVSASVGDRVTITCRASQGIASWLAWYQQKPGKAPELLIYAASSLQGGVPSRFSGSGSGTDFTLTISSLQPEDFAIYYCQQANYFPWTFGQGTKVEIK

SEQ ID NO: 138 - protein, 6 aa, synthetic construct
QGIASW

SEQ ID NO: 139 - skipped

SEQ ID NO: 140 - protein, 9 aa, synthetic construct
QQANYFPWT

SEQ ID NO: 141 - DNA, 363 bp, synthetic construct
  source 1..363: mol_type = other DNA
gaggtgcagctgttggagtctgggggagccttggtacagcctggggggtccctgagactctcctgtgcagcctctggattcacctttacctcctatgccatgcactgggtccgccaggctccagggaagggtctggagtgggtctcatctattagaggtagtggtggtggcacatactccgcagactccgtgaagggccggttcaccatctccagagacaattccagggacactctatatctgcaaatgaacagtgtgagagccgaggacacggccgtttattactgtgcgaggtcccatgactacggtgccttcgacttctttgactactggggccagggaaccctggtcaccgtctcctca

SEQ ID NO: 142 - protein, 121 aa, synthetic construct
EVQLLESGGALVQPGGSLRLSCAASGFTFTSYAMHWVRQAPGKGLEWVSSIRGSGGGTYSADSVKGRFTISRDNSRDTLYLQMNSVRAEDTAVYYCARSHDYGAFDFFDYWGQGTLVTVSS

SEQ ID NO: 143 - protein, 8 aa, synthetic construct
GFTFTSYA

SEQ ID NO: 144 - protein, 8 aa, synthetic construct
IRGSGGGT

SEQ ID NO: 145 - protein, 14 aa, synthetic construct
ARSHDYGAFDFFDY

SEQ ID NO: 146 - DNA, 321 bp, synthetic construct
  source 1..321: mol_type = other DNA
gacatccagatgacccagtctccatcctccctgtctgcatctgtaggagacagagtcaccatcacttgccgggcaagtcagggcattagaactgatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcggcctgaagattttgcaactttttactgtctacagtataatagttacccgctcactttcggcggagggaccaaggtggagatcaaa

SEQ ID NO: 147 - protein, 107 aa, synthetic construct
DIQMTQSPSSLSASVGDRVTITCRASQGIRTDLGWYQQKPGKAPKRLIYAASSLQSGVPSRFSGSGSGTEFTLTISSLRPEDFATFYCLQYNSYPLTFGGGTKVEIK

SEQ ID NO: 148 - protein, 6 aa, synthetic construct
QGIRTD

SEQ ID NO: 149 - skipped

SEQ ID NO: 150 - protein, 9 aa, synthetic construct
LQYNSYPLT

SEQ ID NO: 151 - DNA, 348 bp, synthetic construct
  source 1..348: mol_type = other DNA
caggtgcagctggtggagtctgggggaggcttggtcaagcctggagggtccctgagactctcctgtgcagcctctggattcaccttcagtgactacttcatgagctggatccgccaggctccagggaaggggctggagtgggtttcatacattagtagtactggtagtaccataaattatgcagactctgtgaagggccgattcaccatctccagggacaatgtcaagaattcactgtatctgcaaatgaccagcctgagagtcgaggacacggccgtgtattactgtacgagagataactggaactatgaatactggggccagggaaccctggtcaccgtctcctca

SEQ ID NO: 152 - protein, 116 aa, synthetic construct
QVQLVESGGGLVKPGGSLRLSCAASGFTFSDYFMSWIRQAPGKGLEWVSYISSTGSTINYADSVKGRFTISRDNVKNSLYLQMTSLRVEDTAVYYCTRDNWNYEYWGQGTLVTVSS

SEQ ID NO: 153 - protein, 8 aa, synthetic construct
GFTFSDYF

SEQ ID NO: 154 - protein, 8 aa, synthetic construct
ISSTGSTI

SEQ ID NO: 155 - protein, 9 aa, synthetic construct
TRDNWNYEY

SEQ ID NO: 156 - DNA, 321 bp, synthetic construct
  source 1..321: mol_type = other DNA
gaaatagtgatgacgcagtctccagccaccctgtctgtgtctccaggggaaagagccaccctctcctgcagggccagtcagagtgttagcatcaacttagcctggtaccagcagaaacctggccaggctcccaggctcctcatctttgttgcatccaccagggccactggtatcccagccaggttcagtggcagtgggtctgggacagagttcactctcaccatcagcagcctgcagtctgaagattttgcaacttattactgtcagcagtatgatatctggccgtacacttttggccaggggaccaagctggagatcaaa

SEQ ID NO: 157 - protein, 107 aa, synthetic construct
EIVMTQSPATLSVSPGERATLSCRASQSVSINLAWYQQKPGQAPRLLIFVASTRATGIPARFSGSGSGTEFTLTISSLQSEDFATYYCQQYDIWPYTFGQGTKLEIK

SEQ ID NO: 158 - protein, 6 aa, synthetic construct
QSVSIN

SEQ ID NO: 159 - skipped

SEQ ID NO: 160 - protein, 9 aa, synthetic construct
QQYDIWPYT

SEQ ID NO: 161 - DNA, 369 bp, synthetic construct
  source 1..369: mol_type = other DNA
cagctgcagctgcaggagtcgggcccaggactggtgaagccttcggagaccctgtccctcacctgcactgtgtctggtgaatccatcagcagtaatacttactactggggctggatccgccagcccccagggaaggggctggaatggattgggagtatcgattatagtgggaccaccaattataacccgtccctcaagagtcgagtcaccatatccgtagacacgtccaggaatcacttctccctgaggctgaggtctgtgaccgccgcagacacggctgtgtattactgtgcgagagagtggggaaactacggctactattacggtatggacgtttggggccaagggaccacggtcaccgtctcctca

SEQ ID NO: 162 - protein, 123 aa, synthetic construct
QLQLQESGPGLVKPSETLSLTCTVSGESISSNTYYWGWIRQPPGKGLEWIGSIDYSGTTNYNPSLKSRVTISVDTSRNHFSLRLRSVTAADTAVYYCAREWGNYGYYYGMDVWGQGTTVTVSS

SEQ ID NO: 163 - protein, 10 aa, synthetic construct
GESISSNTYY

SEQ ID NO: 164 - protein, 7 aa, synthetic construct
IDYSGTT

SEQ ID NO: 165 - protein, 15 aa, synthetic construct
AREWGNYGYYYGMDV

SEQ ID NO: 166 - DNA, 321 bp, synthetic construct
  source 1..321: mol_type = other DNA
gacatccagatgacccagtctccatcctccctgtctgcatctgtaggagacagagtcaccatcaattgccgggcaagtcagggcattagaaatgatttaggctggtatcagcagaaaccagggaaagcccctaagcgcctgatctatgctgcatccagtttgcaaagtggggtcccattaaggttcagtggcagtggatctgggacagaattcactctcacaatcaacaacctgcagcctgaagattttgcaacttattactgtctatcgcataatagttacccgtggacgttcggccaagggaccaaggtggaaatcaaa

SEQ ID NO: 167 - protein, 107 aa, synthetic construct
DIQMTQSPSSLSASVGDRVTINCRASQGIRNDLGWYQQKPGKAPKRLIYAASSLQSGVPLRFSGSGSGTEFTLTINNLQPEDFATYYCLSHNSYPWTFGQGTKVEIK

SEQ ID NO: 168 - protein, 6 aa, synthetic construct
QGIRND

SEQ ID NO: 169 - skipped

SEQ ID NO: 170 - protein, 9 aa, synthetic construct
LSHNSYPWT

SEQ ID NO: 171 - DNA, 354 bp, synthetic construct
  source 1..354: mol_type = other DNA
caggtgcagctggtgcagtctggggctgaggtgaagaagcctgggtcctcggtgagggtctcctgcaaggcttctagaggcaccttcagcagctatgctatcagctgggtgcgacaggcccctggacaaggccttgagtggatgggagggatcatccccatctttggtacagcaaactacgcacagaagttcctggccagagtcacgattaccgcggacgaatccacgagcacagcctacatggagctgagcagcctgagatctgaggacacggccgtgtattactgtgcgagagagaaggggtggaactactttgactactggggccagggaaccctggtcaccgtctcctca

SEQ ID NO: 172 - protein, 118 aa, synthetic construct
QVQLVQSGAEVKKPGSSVRVSCKASRGTFSSYAISWVRQAPGQGLEWMGGIIPIFGTANYAQKFLARVTITADESTSTAYMELSSLRSEDTAVYYCAREKGWNYFDYWGQGTLVTVSS

SEQ ID NO: 173 - protein, 8 aa, synthetic construct
RGTFSSYA

SEQ ID NO: 174 - protein, 8 aa, synthetic construct
IIPIFGTA

SEQ ID NO: 175 - protein, 11 aa, synthetic construct
AREKGWNYFDY

SEQ ID NO: 176 - DNA, 321 bp, synthetic construct
  source 1..321: mol_type = other DNA
gacatccagatgacccagtctccaccttccgtgtctgcatctgtaggagacagagtcaccatcacttgtcgggcgagtcagggtattagcagctggttagcctggtatcagcagaaaccagggaaagcccctaaactcctgatctatgctgcatccagtttgcaaagtggggtcccatcaaggttcagcggcagtggatctgggacagatttcactctcaccatcagcagcctgcagcctgaagattttgcaacttactattgtcaacaggctaacagtttccctcggacgttcggccaagggaccaaggtggaaatcaaa

SEQ ID NO: 177 - protein, 107 aa, synthetic construct
DIQMTQSPPSVSASVGDRVTITCRASQGISSWLAWYQQKPGKAPKLLIYAASSLQSGVPSRFSGSGSGTDFTLTISSLQPEDFATYYCQQANSFPRTFGQGTKVEIK

SEQ ID NO: 178 - protein, 6 aa, synthetic construct
QGISSW

SEQ ID NO: 179 - skipped

SEQ ID NO: 180 - protein, 9 aa, synthetic construct
QQANSFPRT

SEQ ID NO: 181 - DNA, 369 bp, synthetic construct
  source 1..369: mol_type = other DNA
caggtgcagctggtggagtctgggggaggcttggtcaagcctggagggtccctgagactctcctgtgcagcctctggattcaccttcagtgactactacatgaactggatccgccaggctccagggaaggggctggagtgggtttcatacattagtagtagtgggactaccatatactacgcagactctgtgaagggccgattcaccatctccagggacaacgccaagaaatcactgtatctggagatgaacagcctcagagccgaggacacggccgtgtactactgtgcgagagaggggtacggtaatgactactattactacggtatagacgtctggggccaagggaccacggtcaccgtctcctca

SEQ ID NO: 182 - protein, 123 aa, synthetic construct
QVQLVESGGGLVKPGGSLRLSCAASGFTFSDYYMNWIRQAPGKGLEWVSYISSSGTTIYYADSVKGRFTISRDNAKKSLYLEMNSLRAEDTAVYYCAREGYGNDYYYYGIDVWGQGTTVTVSS

SEQ ID NO: 183 - protein, 8 aa, synthetic construct
GFTFSDYY

SEQ ID NO: 184 - protein, 8 aa, synthetic construct
ISSSGTTI

SEQ ID NO: 185 - protein, 16 aa, synthetic construct
AREGYGNDYYYYGIDV

SEQ ID NO: 186 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatggtaatggatacaactatttgacttggtacctgcagaagccagggcagtctccacagctcctgatctatttgggttctaatcgggcctccggggtccctgacaggttcagtggcagtggatcaggcacagattttacactgaaaataagcagagtggaggctgaggatgttggggtttattactgcatgcaagctctacaaactccgtacacttttggccaggggaccaagctggagatcaaa

SEQ ID NO: 187 - protein, 112 aa, synthetic construct
DIVMTQSPLSLPVTPGEPASISCRSSQSLLHGNGYNYLTWYLQKPGQSPQLLIYLGSNRASGVPDRFSGSGSGTDFTLKISRVEAEDVGVYYCMQALQTPYTFGQGTKLEIK

SEQ ID NO: 188 - protein, 11 aa, synthetic construct
QSLLHGNGYNY

SEQ ID NO: 189 - skipped

SEQ ID NO: 190 - protein, 9 aa, synthetic construct
MQALQTPYT

SEQ ID NO: 191 - DNA, 366 bp, synthetic construct
  source 1..366: mol_type = other DNA
caggtgcagctggtggagtctgggggaggcgtggtccagcctgggaggtccctgagactctcctgtgcagcctctggattcaccttcagtagctttggcatgcactgggtccgccaggctccaggcaaggggctggagtgggtgatatttatatcatatgatggaagtgataaatactatgcagactccgtgaagggccgattcgccatctccagagacagttccaagaacacgctatatctgcaaatgaacagcctgagagctgaggacacggctgtgtattactgtgcgaaagaaaacggtattttgactgattcctacggtatggacgtctggggccaagggaccacggtcaccgtctcctca

SEQ ID NO: 192 - protein, 122 aa, synthetic construct
QVQLVESGGGVVQPGRSLRLSCAASGFTFSSFGMHWVRQAPGKGLEWVIFISYDGSDKYYADSVKGRFAISRDSSKNTLYLQMNSLRAEDTAVYYCAKENGILTDSYGMDVWGQGTTVTVSS

SEQ ID NO: 193 - protein, 8 aa, synthetic construct
GFTFSSFG

SEQ ID NO: 194 - protein, 8 aa, synthetic construct
ISYDGSDK

SEQ ID NO: 195 - protein, 15 aa, synthetic construct
AKENGILTDSYGMDV

SEQ ID NO: 196 - DNA, 324 bp, synthetic construct
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 197 - protein, 108 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 198 - protein, 6 aa, synthetic construct
QSISSY

SEQ ID NO: 199 - skipped

SEQ ID NO: 200 - protein, 10 aa, synthetic construct
QQSYSTPPIT

SEQ ID NO: 201 - DNA, 372 bp, synthetic construct
  source 1..372: mol_type = other DNA
caggttcagctggtgcagtctggagctgaggtgaagaagcctggggcctctgtgaaggtctcctgcaaggcttctggttacacctttaccagctatggtatcagctgggtgcgacaggcccctggacaagggcttgagtggatgggatggatcagtgtttaccatggtaacacaaactatgcacagaagttccagggcagagtcaccatgaccacagacacatccacgagcacagcctacatggagctgaggagcctgagatctgacgacacggccgtgtattactgtgcgagagaggggtattacgatttttggagtggttattacccttttgactactggggccagggaaccctggtcaccgtctcctca

SEQ ID NO: 202 - protein, 124 aa, synthetic construct
QVQLVQSGAEVKKPGASVKVSCKASGYTFTSYGISWVRQAPGQGLEWMGWISVYHGNTNYAQKFQGRVTMTTDTSTSTAYMELRSLRSDDTAVYYCAREGYYDFWSGYYPFDYWGQGTLVTVSS

SEQ ID NO: 203 - protein, 8 aa, synthetic construct
GYTFTSYG

SEQ ID NO: 204 - protein, 8 aa, synthetic construct
ISVYHGNT

SEQ ID NO: 205 - protein, 17 aa, synthetic construct
AREGYYDFWSGYYPFDY

SEQ ID NO: 206 - DNA, 324 bp, synthetic construct; identical to SEQ ID NO: 196
  source 1..324: mol_type = other DNA

SEQ ID NO: 207 - protein, 108 aa, synthetic construct; identical to SEQ ID NO: 197

SEQ ID NO: 208 - protein, 6 aa, synthetic construct
QSISSY

SEQ ID NO: 209 - skipped

SEQ ID NO: 210 - protein, 10 aa, synthetic construct
QQSYSTPPIT

SEQ ID NO: 211 - DNA, 354 bp, synthetic construct
  source 1..354: mol_type = other DNA
gaggtgcagctggtggagtctgggggaggcttgatacaacctggagggtccctgagactctcctgtgaagcctctggattcaccttcagaaattatgaaatgaattgggtccgccaggctccagggaaggggctggagtgggtttcatatattagtagtagtggtaatatgaaagactacgcagagtctgtgaagggccgattcaccatctccagagacaatgtcaagaattcactgcagctgcaaatgaacagcctgagagtcgaggacacggctgtttattactgtgcgagagacgagtttccttacggaatggacgtctggggccaagggaccacggtcaccgtctcctca

SEQ ID NO: 212 - protein, 118 aa, synthetic construct
EVQLVESGGGLIQPGGSLRLSCEASGFTFRNYEMNWVRQAPGKGLEWVSYISSSGNMKDYAESVKGRFTISRDNVKNSLQLQMNSLRVEDTAVYYCARDEFPYGMDVWGQGTTVTVSS

SEQ ID NO: 213 - protein, 8 aa, synthetic construct
GFTFRNYE

SEQ ID NO: 214 - protein, 8 aa, synthetic construct
ISSSGNMK

SEQ ID NO: 215 - protein, 11 aa, synthetic construct
ARDEFPYGMDV

SEQ ID NO: 216 - DNA, 324 bp, synthetic construct; identical to SEQ ID NO: 196
  source 1..324: mol_type = other DNA

SEQ ID NO: 217 - protein, 108 aa, synthetic construct; identical to SEQ ID NO: 197

SEQ ID NO: 218 - protein, 6 aa, synthetic construct
QSISSY

SEQ ID NO: 219 - skipped

SEQ ID NO: 220 - protein, 10 aa, synthetic construct
QQSYSTPPIT

SEQ ID NO: 221 - DNA, 363 bp, synthetic construct
  source 1..363: mol_type = other DNA
gaagtgcagctggtggagtctgggggaggcttggttcagcctggcaggtccctgagactctcctgtgcagcctctggattcacctttgatgattatgccatgaactgggtccggcaagctccagggaagggcctggagtgggtctcaggtattagttggagtagtggtagcatggactatgcggactctgtgaagggccgattcaccatctccagagacaacgccaaaaactccctgtatctgcaaatgaacagtctgagaactgaggacacggccttatattactgtgcaaaagctagggaagttggagactactacggtatggacgtctggggccaagggaccacggtcaccgtctcctca

SEQ ID NO: 222 - protein, 121 aa, synthetic construct
EVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSS

SEQ ID NO: 223 - protein, 8 aa, synthetic construct
GFTFDDYA

SEQ ID NO: 224 - protein, 8 aa, synthetic construct
ISWSSGSM

SEQ ID NO: 225 - protein, 14 aa, synthetic construct
AKAREVGDYYGMDV

SEQ ID NO: 226 - DNA, 324 bp, synthetic construct; identical to SEQ ID NO: 196
  source 1..324: mol_type = other DNA

SEQ ID NO: 227 - protein, 108 aa, synthetic construct; identical to SEQ ID NO: 197

SEQ ID NO: 228 - protein, 6 aa, synthetic construct
QSISSY

SEQ ID NO: 229 - skipped

SEQ ID NO: 230 - protein, 10 aa, synthetic construct
QQSYSTPPIT

SEQ ID NO: 231 - DNA, 357 bp, synthetic construct
  source 1..357: mol_type = other DNA
gaagtgcagctggtggagtctgggggaggcttggtacagcctggcaggtccctgacactctcctgtgcagcctctggattcacctttgataattttggcatgcactgggtccggcaaggtccagggaagggcctggaatgggtctcaggtcttacttggaatagtggtgtcataggctatgcggactctgtgaagggccgattcaccatctccagagacaacgccaagaactccctgtatctgcaaatgaacagtctgagacctgaggacacggccttatattactgtgcaaaagatatacggaattacggcccctttgactactggggccagggaaccctggtcaccgtctcctca

SEQ ID NO: 232 - protein, 119 aa, synthetic construct
EVQLVESGGGLVQPGRSLTLSCAASGFTFDNFGMHWVRQGPGKGLEWVSGLTWNSGVIGYADSVKGRFTISRDNAKNSLYLQMNSLRPEDTALYYCAKDIRNYGPFDYWGQGTLVTVSS

SEQ ID NO: 233 - protein, 8 aa, synthetic construct
GFTFDNFG

SEQ ID NO: 234 - protein, 8 aa, synthetic construct
LTWNSGVI

SEQ ID NO: 235 - protein, 12 aa, synthetic construct
AKDIRNYGPFDY

SEQ ID NO: 236 - DNA, 324 bp, synthetic construct
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccttggacgttcggccaagggaccaaggtggaaatcaaa

SEQ ID NO: 237 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 238 - protein, 7 aa, synthetic construct
QSVSSSY

SEQ ID NO: 239 - skipped

SEQ ID NO: 240 - protein, 9 aa, synthetic construct
QQYGSSPWT

SEQ ID NO: 241 - DNA, 357 bp, synthetic construct
  source 1..357: mol_type = other DNA
gaggtgcagctggtggagtctgggggaggcttagtacagcctggagggtccctaagactctcctgtgcagcctctggattcaccttcaatatttttgaaatgaactgggtccgccaggctccagggaaggggctggagtggatttcctacattagtagtcgtggaactaccacatactacgcagactctgtgaggggccgattcaccatctccagagacaacgccaagaactcactgtatctgcaaatgaacagcctgagagccgaggacacggctgtttattactgtgcgagagattatgaagcaacaatcccttttgacttctggggccagggaaccctggtcaccgtctcctca

SEQ ID NO: 242 - protein, 119 aa, synthetic construct
EVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSS

SEQ ID NO: 243 - protein, 8 aa, synthetic construct
GFTFNIFE

SEQ ID NO: 244 - protein, 8 aa, synthetic construct
ISSRGTTT

SEQ ID NO: 245 - protein, 12 aa, synthetic construct
ARDYEATIPFDF

SEQ ID NO: 246 - DNA, 324 bp, synthetic construct; identical to SEQ ID NO: 196
  source 1..324: mol_type = other DNA

SEQ ID NO: 247 - protein, 108 aa, synthetic construct; identical to SEQ ID NO: 197

SEQ ID NO: 248 - protein, 6 aa, synthetic construct
QSISSY

SEQ ID NO: 249 - skipped

SEQ ID NO: 250 - protein, 10 aa, synthetic construct
QQSYSTPPIT

SEQ ID NO: 251 - DNA, 363 bp, synthetic construct
  source 1..363: mol_type = other DNA
gaggtgcagctggtggagtctgggggaagtgtggtacggcctggggggtccctgagactctcctgtgaagcctctggattcacctttgatgattatggcatgagctgggtccgccaagatccagggaaggggctggagtgggtctctggtattaattggaatggtgatagaacaaattatgcagactctgtgaagggccgattcatcatttccagagacaacgccaagaactctgtgtatctacaaatgaacagtctgagagcggaggactcggccttgtatcactgtgcgagagatcagggactcggagtggcagctacccttgactactggggccagggaaccctggtcaccgtctcctca

SEQ ID NO: 252 - protein, 121 aa, synthetic construct
EVQLVESGGSVVRPGGSLRLSCEASGFTFDDYGMSWVRQDPGKGLEWVSGINWNGDRTNYADSVKGRFIISRDNAKNSVYLQMNSLRAEDSALYHCARDQGLGVAATLDYWGQGTLVTVSS

SEQ ID NO: 253 - protein, 8 aa, synthetic construct
GFTFDDYG

SEQ ID NO: 254 - protein, 8 aa, synthetic construct
INWNGDRT

SEQ ID NO: 255 - protein, 14 aa, synthetic construct
ARDQGLGVAATLDY

SEQ ID NO: 256 - DNA, 324 bp, synthetic construct; identical to SEQ ID NO: 196
  source 1..324: mol_type = other DNA

SEQ ID NO: 257 - protein, 108 aa, synthetic construct; identical to SEQ ID NO: 197

SEQ ID NO: 258 - protein, 6 aa, synthetic construct
QSISSY

SEQ ID NO: 259 - skipped

SEQ ID NO: 260 - protein, 10 aa, synthetic construct
QQSYSTPPIT

SEQ ID NO: 261 - DNA, 384 bp, synthetic construct
  source 1..384: mol_type = other DNA
gaggtgcagctggtggagtctgggggaggcttggtacagcctggagggtccctgagactctcctgtgcagcctctggattcaccgtcagtaattatgaaatgaactgggtccgccaggctccagggaaggggctggagtgggtttcatacattagtagtagtaccagtaacatatactacgcagactctgtgaagggccgattcaccatctccagagacaacgccgagaactcactgtatctgcagatgaacagcctgagagtcgaggacacggctgtttattactgtgtgagagatgggattgtagtagttccagttggtcgtggatactactattacggtttggacgtctggggccaagggaccacggtcaccgtctcctca

SEQ ID NO: 262 - protein, 128 aa, synthetic construct
EVQLVESGGGLVQPGGSLRLSCAASGFTVSNYEMNWVRQAPGKGLEWVSYISSSTSNIYYADSVKGRFTISRDNAENSLYLQMNSLRVEDTAVYYCVRDGIVVVPVGRGYYYYGLDVWGQGTTVTVSS

SEQ ID NO: 263 - protein, 8 aa, synthetic construct
GFTVSNYE

SEQ ID NO: 264 - protein, 8 aa, synthetic construct
ISSSTSNI

SEQ ID NO: 265 - protein, 21 aa, synthetic construct
VRDGIVVVPVGRGYYYYGLDV

SEQ ID NO: 266 - DNA, 324 bp, synthetic construct; identical to SEQ ID NO: 196
  source 1..324: mol_type = other DNA

SEQ ID NO: 267 - protein, 108 aa, synthetic construct; identical to SEQ ID NO: 197

SEQ ID NO: 268 - protein, 6 aa, synthetic construct
QSISSY

SEQ ID NO: 269 - skipped

SEQ ID NO: 270 - protein, 10 aa, synthetic construct
QQSYSTPPIT

SEQ ID NO: 271 - DNA, 378 bp, synthetic construct
  source 1..378: mol_type = other DNA
caggtgcagctggtggagtctgggggaggcgtggtccagcctggaaggtccctgagactctcctgcgcagcctctggattcccctttagtaattatgtcatgtattgggtccgccaggctccaggcaaggggctggagtgggtggctcttattttttttgacggaaagaaaaactatcatgcagactccgtgaagggccgattcaccataaccagagacaattccaaaaatatgttatatctgcaaatgaacagcctgagacctgaggacgcggctgtgtattactgtgcgaaaatccattgtcctaatggtgtatgttacaaggggtattacggaatggacgtctggggccaagggaccacggtcaccgtctcctca

SEQ ID NO: 272 - protein, 126 aa, synthetic construct
QVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSS

SEQ ID NO: 273 - protein, 8 aa, synthetic construct
GFPFSNYV

SEQ ID NO: 274 - protein, 8 aa, synthetic construct
IFFDGKKN

SEQ ID NO: 275 - protein, 19 aa, synthetic construct
AKIHCPNGVCYKGYYGMDV

SEQ ID NO: 276 - DNA, 324 bp, synthetic construct; identical to SEQ ID NO: 196
  source 1..324: mol_type = other DNA

SEQ ID NO: 277 - protein, 108 aa, synthetic construct; identical to SEQ ID NO: 197

SEQ ID NO: 278 - protein, 6 aa, synthetic construct
QSISSY

SEQ ID NO: 279 - skipped

SEQ ID NO: 280 - protein, 10 aa, synthetic construct
QQSYSTPPIT

SEQ ID NO: 281 - DNA, 375 bp, synthetic construct
  source 1..375: mol_type = other DNA
gaggtgcagctggtggagtctgggggaggcttggtccagcctggggggtccctaagactctcctgtgcagcctctggattcacctttagtaactattggatgaactgggtccgccaggctccagggaagggactggagtgggtggccaatataaaagaagatggaggtaagaaattgtatgtggactctgtgaagggccgattcaccatctccagagacaacgccaagaactcactgtttctgcaaatgaacagcctgagagccgaggacacggctgtgtattattgtgcgagagaagatacaactttggttgtggactactactactacggtatggacgtctggggccaagggaccacggtcaccgtctcctca

SEQ ID NO: 282 - protein, 125 aa, synthetic construct
EVQLVESGGGLVQPGGSLRLSCAASGFTFSNYWMNWVRQAPGKGLEWVANIKEDGGKKLYVDSVKGRFTISRDNAKNSLFLQMNSLRAEDTAVYYCAREDTTLVVDYYYYGMDVWGQGTTVTVSS

SEQ ID NO: 283 - protein, 8 aa, synthetic construct
GFTFSNYW

SEQ ID NO: 284 - protein, 8 aa, synthetic construct
IKEDGGKK

SEQ ID NO: 285 - protein, 18 aa, synthetic construct
AREDTTLVVDYYYYGMDV

SEQ ID NO: 286 - DNA, 324 bp, synthetic construct; identical to SEQ ID NO: 196
  source 1..324: mol_type = other DNA

SEQ ID NO: 287 - protein, 108 aa, synthetic construct; identical to SEQ ID NO: 197

SEQ ID NO: 288 - protein, 6 aa, synthetic construct
QSISSY

SEQ ID NO: 289 - skipped

SEQ ID NO: 290 - protein, 10 aa, synthetic construct
QQSYSTPPIT

SEQ ID NO: 291 - DNA, 366 bp, synthetic construct
  source 1..366: mol_type = other DNA
caggtccagctggtgcagtctggggctgaggtgaagaagcctgggtcctcggtgaaggtctcctgcaaggcttctggaggcaccttcaacacctatgctatcacctgggtgcgacaggcccctggacaagggcttgaatggatggggggaatcatccctatctctggcatagcagagtacgcacagaagttccagggcagagtcacgatcaccacggatgactcctcgaccacagcctacatggaactgaacagtctgagatctgaggacacggccgtgtattactgtgcgagctggaactacgcactctactacttctacggtatggacgtctggggccgagggaccacggtcaccgtctcctca

SEQ ID NO: 292 - protein, 122 aa, synthetic construct
QVQLVQSGAEVKKPGSSVKVSCKASGGTFNTYAITWVRQAPGQGLEWMGGIIPISGIAEYAQKFQGRVTITTDDSSTTAYMELNSLRSEDTAVYYCASWNYALYYFYGMDVWGRGTTVTVSS

SEQ ID NO: 293 - protein, 8 aa, synthetic construct
GGTFNTYA

SEQ ID NO: 294 - protein, 8 aa, synthetic construct
IIPISGIA

SEQ ID NO: 295 - protein, 15 aa, synthetic construct
ASWNYALYYFYGMDV

SEQ ID NO: 296 - DNA, 324 bp, synthetic construct; identical to SEQ ID NO: 236
  source 1..324: mol_type = other DNA

SEQ ID NO: 297 - protein, 108 aa, synthetic construct; identical to SEQ ID NO: 237

SEQ ID NO: 298 - protein, 7 aa, synthetic construct
QSVSSSY

SEQ ID NO: 299 - skipped

SEQ ID NO: 300 - protein, 9 aa, synthetic construct
QQYGSSPWT

SEQ ID NO: 301 - DNA, 369 bp, synthetic construct
  source 1..369: mol_type = other DNA
caggtgcagctggtggagtctgggggaggcttggtcaagcctggagggtccctgagactctcctgtgcagcctctggattcaccttcagtgtctattacatgaactggatccgccaggctccagggaagggcctggagtgggtttcatacattagtagtagtggtagtaccatatactacgcagactctgtgaagggccgattcaccatctccagggacaacgccaagaactcactgtatctccaaatgaacagtctgagagccgaggacacggccgtatattactgtgggagagaagggtatagtgggacttattcttattacggtatggacgtctggggccaagggaccacggtcaccgtctcctca

SEQ ID NO: 302 - protein, 123 aa, synthetic construct
QVQLVESGGGLVKPGGSLRLSCAASGFTFSVYYMNWIRQAPGKGLEWVSYISSSGSTIYYADSVKGRFTISRDNAKNSLYLQMNSLRAEDTAVYYCGREGYSGTYSYYGMDVWGQGTTVTVSS

SEQ ID NO: 303 - protein, 8 aa, synthetic construct
GFTFSVYY

SEQ ID NO: 304 - protein, 8 aa, synthetic construct
ISSSGSTI

SEQ ID NO: 305 - protein, 16 aa, synthetic construct
GREGYSGTYSYYGMDV

SEQ ID NO: 306 - DNA, 336 bp, synthetic construct
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagttcctgatctatttgggttctaatcgggcctccggggtccctgacaggttcagtggcagtggatcaggcacagattttacactgaaaatcaacagagtggaggctgaggatgttggggtttattactgcatgcaagctctacaaactccgtacacttttggccaggggaccaagctggagatcaaa

SEQ ID NO: 307 - protein, 112 aa, synthetic construct
DIVMTQSPLSLPVTPGEPASISCRSSQSLLHSNGYNYLDWYLQKPGQSPQFLIYLGSNRASGVPDRFSGSGSGTDFTLKINRVEAEDVGVYYCMQALQTPYTFGQGTKLEIK

SEQ ID NO: 308 - protein, 11 aa, synthetic construct
QSLLHSNGYNY

SEQ ID NO: 309 - skipped

SEQ ID NO: 310 - protein, 9 aa, synthetic construct
MQALQTPYT

SEQ ID NO: 311 - DNA, 366 bp, synthetic construct
  source 1..366: mol_type = other DNA
gaagtgcagctggtggagtctgggggagggttggtacagcctggcaggtccctgagactctcctgtgcagtctctggattcacctttgatgattatgccatgcactgggtccggcaagctccagggaagggcctggagtgggtctcaggtattagttggaatagtggtaccagaggatatgcggactctgtgaagggccgattcaccatctccagagacaacgccaagaactccctgtatctgcaaatgaacagtctgagaggtgaggacacggccttgtattactgtgtaaaagatattacgatatcccccaactactacggtatggacgtctggggccaagggaccacggtcaccgtctcctca

SEQ ID NO: 312 - protein, 122 aa, synthetic construct
EVQLVESGGGLVQPGRSLRLSCAVSGFTFDDYAMHWVRQAPGKGLEWVSGISWNSGTRGYADSVKGRFTISRDNAKNSLYLQMNSLRGEDTALYYCVKDITISPNYYGMDVWGQGTTVTVSS

SEQ ID NO: 313 - protein, 8 aa, synthetic construct
GFTFDDYA

SEQ ID NO: 314 - protein, 8 aa, synthetic construct
ISWNSGTR

SEQ ID NO: 315 - protein, 15 aa, synthetic construct
VKDITISPNYYGMDV

SEQ ID NO: 316 - DNA, 321 bp, synthetic construct
  source 1..321: mol_type = other DNA
gacatccagatgacccagtctccatcctccctgtctgcatctgtaggagacagagtcaccatcacttgccgggcgagtcaggacattagccattattcagcctggtatcagcagaaaccagggaaacttcctaacctcctgatctatgctgcatccactttgcaatcaggggtcccatctcggttcagtggcagtggatctgggacagatttctctctcaccaccagcagcctgcagcctgaagatgttgcaacttattactgtcaaaagtataacagtgtccctctcactttcggcggagggaccaaggtggagatcaaa

SEQ ID NO: 317 - protein, 107 aa, synthetic construct
DIQMTQSPSSLSASVGDRVTITCRASQDISHYSAWYQQKPGKLPNLLIYAASTLQSGVPSRFSGSGSGTDFSLTTSSLQPEDVATYYCQKYNSVPLTFGGGTKVEIK

SEQ ID NO: 318 - protein, 6 aa, synthetic construct
QDISHY

SEQ ID NO: 319 - skipped

SEQ ID NO: 320 - protein, 9 aa, synthetic construct
QKYNSVPLT

SEQ ID NO: 321 - protein, 1168 aa, synthetic construct
MHRPRRRGTRPPPLALLAALLLAARGADADIQMTQSPSSVSASVGDRVTITCRASQGIASWLAWYQQKPGKAPELLIYAASSLQGGVPSRFSGSGSGTDFTLTISSLQPEDFAIYYCQQANYFPWTFGQGTKVEIKGGGGSGGGGSGGGGSQITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 322 - protein, 1171 aa, synthetic construct
MHRPRRRGTRPPPLALLAALLLAARGADADIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 323 - protein, 1164 aa, synthetic construct
MHRPRRRGTRPPPLALLAALLLAARGADADIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 324 - protein, 1166 aa, synthetic construct
MHRPRRRGTRPPPLALLAALLLAARGADADIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 325 - protein, 883 aa, synthetic construct
AHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 326 - protein, 118 aa, synthetic construct
EVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSS

SEQ ID NO: 327 - protein, 107 aa, synthetic construct
DIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELK

SEQ ID NO: 328 - protein, 214 aa, synthetic construct
DIQMTQSPSSLSASVGDRVTITCRASQGISNYLAWYQQKPGKVPNLLIYAASTLQSGVPSRFSGSGSGTDFTLTISSLQPEDVATYYCQKYNSAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 329 - protein, 228 aa, synthetic construct
EVQLVESGGGLVQPGGSLRLSCAASGFAFSSYAMTWVRQAPGKGLEWVSVISGTGGSTYYADSVKGRFTISRDNSKNTLYLQMNSLRAEDTAVYYCAKGGAARRMEYFQYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 330 - protein, 214 aa, synthetic construct
DIQMTQSPSSLSASIGDRVTITCRASQGISNYLAWYQQKPGKVPKLLIYAASTLQSGVPSRFSGSGSGTDFTLTISSLQPEDVATYYCQNHNSVPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 331 - protein, 228 aa, synthetic construct
EVQLVESGGGLVQPGGSLRLSCAASGFTFNSYAMTWVRQAPGKGLEWVSFIGGSTGNTYYAGSVKGRFTISSDNSKKTLYLQMNSLRAEDTAVYYCAKGGAARRMEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 332 - protein, 214 aa, synthetic construct
DIQMTQSPSSLSASVGDRVTITCRASQSIRNVLGWFQQKPGKAPQRLIYAASSLQSGVPSRFSGSGSGTEFTLTISSLQPEDFATYYCLQHNFYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 333 - protein, 229 aa, synthetic construct
QVQLVESGGGVVQPGRSLRLSCAASGFTFTTYGMHWVRQAPGKGLEWVAVIWYDGSNKYYGDSVKGRFTISRDNSKNTLYLQMNSLRVDDTAVYYCTRTHGYTRSSDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 334 - protein, 214 aa, synthetic construct
EIVLTQSPATLSLSPGERATLSCRASQSVSSYLAWYQQKPGQAPRLLIHDVSNRATGIPARFSGSGSGTDFTLTISSLEPEDFVVYYCQQRSDWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 335 - protein, 230 aa, synthetic construct
EVQLVESGGGLVQPGRSLRLSCAASGFTFDDKAMHWVRQVPGKGLEWISGISWNSGTIGYADSVKGRFIISRDNAKNSLYLQMNSLRAEDTALYYCAKDGDTSGWYWYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 336 - protein, 214 aa, synthetic construct
DIQLTQSPSSLSASVGDRVTITCWASQGISSYLAWYQQKPGKAPKLLIYAASTLQSGVPSRFSGSGSGTEFTLTISSLQPEDFATYYCQQLNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 337 - protein, 227 aa, synthetic construct
QVQLVESGGGVVQPGRSLRLSCIASGFTFSVYGIHWVRQAPGKGLEWMAVISHDGNIKHYADSVKGRFTISRDNSKNTLYLQINSLRTEDTAVYYCAKDTWNSLDTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 338 - protein, 214 aa, synthetic construct
AIQMTQSPSSLSASVGDRVTITCRASQGIRNDLGWYQQKPGKAPKLLIYAASTLQSGVPSRFSGSGSGTDFTLTISSLQPEDFATYYCLQDYNYPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 339 - protein, 227 aa, synthetic construct
QVTLRESGPALVKPSQTLTLTCTFSGFSLNTYGMFVSWIRQPPGKALEWLAHIHWDDDKYYSTSLKTRLTISKDTSKNQVVLTMTNMDPVDTATYYCARGHNNLNYIIHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 340 - protein, 214 aa, synthetic construct
EIVMTQSPATLSVSPGERATLSCRASQSVSSNFAWYQQKPGQAPRLLIYSASSRATGIPVRFSGSGSGTEFTLTISSLQSEDFAVYYCQQYNIWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 341 - protein, 223 aa, synthetic construct
EVQLVESGGGLVQPGGSLRLSCAVSGFIFSSYEMNWVRQAPGKGLEWVSYISSSGSTIFYADSVKGRFTISRDNAKNSLYLQMNSLRAEDTAVYYCVSGVVLFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 342 - protein, 214 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 343 - protein, 232 aa, synthetic construct
EVQLVESGGGLVQPGGSLRLSCAASGFTFSNYWMTWVRQAPGKGLEWVANIKEDGSEKDYVDSVKGRFTISRDNAKNSLYLQMNSLRGEDTAVYYCARDGEQLVDYYYYYVMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 344 - protein, 214 aa, synthetic construct
DIQMTQSPSSLSASVGDRVTITCRASQSISSYLNWYQQKPGKAPKVLIYAASSLQSGVPSRFSGSGSGTDFTLTISSLQPEDFATYYCQQSYSIPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 345 - protein, 234 aa, synthetic construct
EVQLVESGGGLVQPGRSLRLSCAASGFTFDDYAMHWVRQAPGKGLEWVSGISWNSGYIGYADSVKGRFTISRDNAENSLHLQMNSLRAEDTALYYCARGGSTLVRGVKGGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 346 - protein, 214 aa, synthetic construct
DIQMTQSPSSLSASVGDRVTITCRASQSIDRYLNWYRQKPGKAPKLLIYTTSSLQSGVPSRFSGSGSGTDFTLTLSSLQPEDFATYYCQQSYSPPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 347 - protein, 224 aa, synthetic construct
QVQLVESGGGVVQPGRSLRLSCAASGFTFSSYGMHWVRQAPGKGLEWVAVIWYDGSNKYYADSVKGRFTISRDISKNTLYLQMNSLRAEDTAVYYCAGQLDLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 348 - protein, 214 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKADSLPYAFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 349 - protein, 232 aa, synthetic construct
EVQLVESGGGLVQPGGSLRLSCTASGFTFSNYWMTWVRQAPGKGLEWVANIKEDGSEKEYVDSVKGRFTISRDNAKNSLYLQMNSLRGEDTAVYYCARDGEQLVDYYYYYVMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 350 - protein, 214 aa, synthetic construct
DIQMTQSPSSLSASVGDRVTITCRASQGIRDHFGWYQQKPGKAPKRLIYAASSLHSGVPSRFSGSGSGTEFTLTISSLQPEDFATYYCLQYDTYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 351 - protein, 228 aa, synthetic construct
EVQLVESGGGLVQPGGSLRLSCATSGFTFTSYDMKWVRQAPGLGLEWVSAISGSGGNTYYADSVKGRFTISRDNSRNTLYLQMNSLRAEDTAVYYCTRSHDFGAFDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 352 - protein, 215 aa, synthetic construct
EIVMTQSPATLSVSPGERATLSCRASQTVSSNLAWYQQKPGQAPRLLIYGSSSRATGIPARFSGSGSGTEFTLTISSLQSEDFAVYYCQQYNNWPPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 353 - protein, 229 aa, synthetic construct
EVQLVESGGDLVQPGRSLRLSCAASGFTFDDYAMHWVRQAPGKGLEWVSGISWNSATRVYADSVKGRFTISRDNAKNFLYLQMNSLRSEDTALYHCAKDMDISLGYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 354 - protein, 214 aa, synthetic construct
DIQMTQSPSSVSASVGDRVTITCRASQGIASWLAWYQQKPGKAPELLIYAASSLQGGVPSRFSGSGSGTDFTLTISSLQPEDFAIYYCQQANYFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 355 - protein, 231 aa, synthetic construct
QITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 356 - protein, 214 aa, synthetic construct
DIQMTQSPSSLSASVGDRVTITCRASQGIRTDLGWYQQKPGKAPKRLIYAASSLQSGVPSRFSGSGSGTEFTLTISSLRPEDFATFYCLQYNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 357 - protein, 228 aa, synthetic construct
EVQLLESGGALVQPGGSLRLSCAASGFTFTSYAMHWVRQAPGKGLEWVSSIRGSGGGTYSADSVKGRFTISRDNSRDTLYLQMNSVRAEDTAVYYCARSHDYGAFDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 358 - protein, 214 aa, synthetic construct
EIVMTQSPATLSVSPGERATLSCRASQSVSINLAWYQQKPGQAPRLLIFVASTRATGIPARFSGSGSGTEFTLTISSLQSEDFATYYCQQYDIWPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 359 - protein, 223 aa, synthetic construct
QVQLVESGGGLVKPGGSLRLSCAASGFTFSDYFMSWIRQAPGKGLEWVSYISSTGSTINYADSVKGRFTISRDNVKNSLYLQMTSLRVEDTAVYYCTRDNWNYEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 360 - protein, 214 aa, synthetic construct
DIQMTQSPSSLSASVGDRVTINCRASQGIRNDLGWYQQKPGKAPKRLIYAASSLQSGVPLRFSGSGSGTEFTLTINNLQPEDFATYYCLSHNSYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 361 - protein, 230 aa, synthetic construct
QLQLQESGPGLVKPSETLSLTCTVSGESISSNTYYWGWIRQPPGKGLEWIGSIDYSGTTNYNPSLKSRVTISVDTSRNHFSLRLRSVTAADTAVYYCAREWGNYGYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 362 - protein, 214 aa, synthetic construct
DIQMTQSPPSVSASVGDRVTITCRASQGISSWLAWYQQKPGKAPKLLIYAASSLQSGVPSRFSGSGSGTDFTLTISSLQPEDFATYYCQQANSFPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 363 - protein, 225 aa, synthetic construct
QVQLVQSGAEVKKPGSSVRVSCKASRGTFSSYAISWVRQAPGQGLEWMGGIIPIFGTANYAQKFLARVTITADESTSTAYMELSSLRSEDTAVYYCAREKGWNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 364 - protein, 219 aa, synthetic construct
DIVMTQSPLSLPVTPGEPASISCRSSQSLLHGNGYNYLTWYLQKPGQSPQLLIYLGSNRASGVPDRFSGSGSGTDFTLKISRVEAEDVGVYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 365 - protein, 230 aa, synthetic construct
QVQLVESGGGLVKPGGSLRLSCAASGFTFSDYYMNWIRQAPGKGLEWVSYISSSGTTIYYADSVKGRFTISRDNAKKSLYLEMNSLRAEDTAVYYCAREGYGNDYYYYGIDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 366 - protein, 215 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 367 - protein, 229 aa, synthetic construct
QVQLVESGGGVVQPGRSLRLSCAASGFTFSSFGMHWVRQAPGKGLEWVIFISYDGSDKYYADSVKGRFAISRDSSKNTLYLQMNSLRAEDTAVYYCAKENGILTDSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 368 - protein, 215 aa, synthetic construct; identical to SEQ ID NO: 366

SEQ ID NO: 369 - protein, 231 aa, synthetic construct
QVQLVQSGAEVKKPGASVKVSCKASGYTFTSYGISWVRQAPGQGLEWMGWISVYHGNTNYAQKFQGRVTMTTDTSTSTAYMELRSLRSDDTAVYYCAREGYYDFWSGYYPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 370 - protein, 215 aa, synthetic construct; identical to SEQ ID NO: 366

SEQ ID NO: 371 - protein, 225 aa, synthetic construct
EVQLVESGGGLIQPGGSLRLSCEASGFTFRNYEMNWVRQAPGKGLEWVSYISSSGNMKDYAESVKGRFTISRDNVKNSLQLQMNSLRVEDTAVYYCARDEFPYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 372 - protein, 215 aa, synthetic construct; identical to SEQ ID NO: 366

SEQ ID NO: 373 - protein, 228 aa, synthetic construct
EVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 374 - protein, 215 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 375 - protein, 226 aa, synthetic construct
EVQLVESGGGLVQPGRSLTLSCAASGFTFDNFGMHWVRQGPGKGLEWVSGLTWNSGVIGYADSVKGRFTISRDNAKNSLYLQMNSLRPEDTALYYCAKDIRNYGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 376 - protein, 215 aa, synthetic construct; identical to SEQ ID NO: 366

SEQ ID NO: 377 - protein, 226 aa, synthetic construct
EVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 378 - protein, 215 aa, synthetic construct; identical to SEQ ID NO: 366

SEQ ID NO: 379 - protein, 228 aa, synthetic construct
EVQLVESGGSVVRPGGSLRLSCEASGFTFDDYGMSWVRQDPGKGLEWVSGINWNGDRTNYADSVKGRFIISRDNAKNSVYLQMNSLRAEDSALYHCARDQGLGVAATLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 380 - protein, 215 aa, synthetic construct; identical to SEQ ID NO: 366

SEQ ID NO: 381 - protein, 235 aa, synthetic construct
EVQLVESGGGLVQPGGSLRLSCAASGFTVSNYEMNWVRQAPGKGLEWVSYISSSTSNIYYADSVKGRFTISRDNAENSLYLQMNSLRVEDTAVYYCVRDGIVVVPVGRGYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 382 - protein, 215 aa, synthetic construct; identical to SEQ ID NO: 366

SEQ ID NO: 383 - protein, 233 aa, synthetic construct
QVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 384 - protein, 215 aa, synthetic construct; identical to SEQ ID NO: 366

SEQ ID NO: 385 - protein, 232 aa, synthetic construct
EVQLVESGGGLVQPGGSLRLSCAASGFTFSNYWMNWVRQAPGKGLEWVANIKEDGGKKLYVDSVKGRFTISRDNAKNSLFLQMNSLRAEDTAVYYCAREDTTLVVDYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 386 - protein, 215 aa, synthetic construct; identical to SEQ ID NO: 374

SEQ ID NO: 387 - protein, 229 aa, synthetic construct
QVQLVQSGAEVKKPGSSVKVSCKASGGTFNTYAITWVRQAPGQGLEWMGGIIPISGIAEYAQKFQGRVTITTDDSSTTAYMELNSLRSEDTAVYYCASWNYALYYFYGMDVWGRGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 388 - protein, 219 aa, synthetic construct
DIVMTQSPLSLPVTPGEPASISCRSSQSLLHSNGYNYLDWYLQKPGQSPQFLIYLGSNRASGVPDRFSGSGSGTDFTLKINRVEAEDVGVYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 389 - protein, 230 aa, synthetic construct
QVQLVESGGGLVKPGGSLRLSCAASGFTFSVYYMNWIRQAPGKGLEWVSYISSSGSTIYYADSVKGRFTISRDNAKNSLYLQMNSLRAEDTAVYYCGREGYSGTYSYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 390 - protein, 214 aa, synthetic construct
DIQMTQSPSSLSASVGDRVTITCRASQDISHYSAWYQQKPGKLPNLLIYAASTLQSGVPSRFSGSGSGTDFSLTTSSLQPEDVATYYCQKYNSVPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 391 - protein, 229 aa, synthetic construct
EVQLVESGGGLVQPGRSLRLSCAVSGFTFDDYAMHWVRQAPGKGLEWVSGISWNSGTRGYADSVKGRFTISRDNAKNSLYLQMNSLRGEDTALYYCVKDITISPNYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 392 - protein, 1136 aa, synthetic construct
DIQMTQSPSSLSASVGDRVTITCRASQGIRDHFGWYQQKPGKAPKRLIYAASSLHSGVPSRFSGSGSGTEFTLTISSLQPEDFATYYCLQYDTYPLTFGGGTKVEIKGGGGSGGGGSGGGGSEVQLVESGGGLVQPGGSLRLSCATSGFTFTSYDMKWVRQAPGLGLEWVSAISGSGGNTYYADSVKGRFTISRDNSRNTLYLQMNSLRAEDTAVYYCTRSHDFGAFDYFDYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 393 - protein, 1138 aa, synthetic construct
EIVMTQSPATLSVSPGERATLSCRASQTVSSNLAWYQQKPGQAPRLLIYGSSSRATGIPARFSGSGSGTEFTLTISSLQSEDFAVYYCQQYNNWPPYTFGQGTKLEIKGGGGSGGGGSGGGGSEVQLVESGGDLVQPGRSLRLSCAASGFTFDDYAMHWVRQAPGKGLEWVSGISWNSATRVYADSVKGRFTISRDNAKNFLYLQMNSLRSEDTALYHCAKDMDISLGYYGLDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 394 - protein, 1139 aa, synthetic construct
DIQMTQSPSSVSASVGDRVTITCRASQGIASWLAWYQQKPGKAPELLIYAASSLQGGVPSRFSGSGSGTDFTLTISSLQPEDFAIYYCQQANYFPWTFGQGTKVEIKGGGGSGGGGSGGGGSQITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 395 - protein, 1136 aa, synthetic construct
DIQMTQSPSSLSASVGDRVTITCRASQGIRTDLGWYQQKPGKAPKRLIYAASSLQSGVPSRFSGSGSGTEFTLTISSLRPEDFATFYCLQYNSYPLTFGGGTKVEIKGGGGSGGGGSGGGGSEVQLLESGGALVQPGGSLRLSCAASGFTFTSYAMHWVRQAPGKGLEWVSSIRGSGGGTYSADSVKGRFTISRDNSRDTLYLQMNSVRAEDTAVYYCARSHDYGAFDFFDYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 396 - protein, 1131 aa, synthetic construct
EIVMTQSPATLSVSPGERATLSCRASQSVSINLAWYQQKPGQAPRLLIFVASTRATGIPARFSGSGSGTEFTLTISSLQSEDFATYYCQQYDIWPYTFGQGTKLEIKGGGGSGGGGSGGGGSQVQLVESGGGLVKPGGSLRLSCAASGFTFSDYFMSWIRQAPGKGLEWVSYISSTGSTINYADSVKGRFTISRDNVKNSLYLQMTSLRVEDTAVYYCTRDNWNYEYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 397 - protein, 1138 aa, synthetic construct
DIQMTQSPSSLSASVGDRVTINCRASQGIRNDLGWYQQKPGKAPKRLIYAASSLQSGVPLRFSGSGSGTEFTLTINNLQPEDFATYYCLSHNSYPWTFGQGTKVEIKGGGGSGGGGSGGGGSQLQLQESGPGLVKPSETLSLTCTVSGESISSNTYYWGWIRQPPGKGLEWIGSIDYSGTTNYNPSLKSRVTISVDTSRNHFSLRLRSVTAADTAVYYCAREWGNYGYYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 398 - protein, 1133 aa, synthetic construct
DIQMTQSPPSVSASVGDRVTITCRASQGISSWLAWYQQKPGKAPKLLIYAASSLQSGVPSRFSGSGSGTDFTLTISSLQPEDFATYYCQQANSFPRTFGQGTKVEIKGGGGSGGGGSGGGGSQVQLVQSGAEVKKPGSSVRVSCKASRGTFSSYAISWVRQAPGQGLEWMGGIIPIFGTANYAQKFLARVTITADESTSTAYMELSSLRSEDTAVYYCAREKGWNYFDYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 399 - protein, 1143 aa, synthetic construct
DIVMTQSPLSLPVTPGEPASISCRSSQSLLHGNGYNYLTWYLQKPGQSPQLLIYLGSNRASGVPDRFSGSGSGTDFTLKISRVEAEDVGVYYCMQALQTPYTFGQGTKLEIKGGGGSGGGGSGGGGSQVQLVESGGGLVKPGGSLRLSCAASGFTFSDYYMNWIRQAPGKGLEWVSYISSSGTTIYYADSVKGRFTISRDNAKKSLYLEMNSLRAEDTAVYYCAREGYGNDYYYYGIDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 400 - protein, 1138 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVESGGGVVQPGRSLRLSCAASGFTFSSFGMHWVRQAPGKGLEWVIFISYDGSDKYYADSVKGRFAISRDSSKNTLYLQMNSLRAEDTAVYYCAKENGILTDSYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 401 - protein, 1140 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVQSGAEVKKPGASVKVSCKASGYTFTSYGISWVRQAPGQGLEWMGWISVYHGNTNYAQKFQGRVTMTTDTSTSTAYMELRSLRSDDTAVYYCAREGYYDFWSGYYPFDYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 402 - protein, 1134 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLIQPGGSLRLSCEASGFTFRNYEMNWVRQAPGKGLEWVSYISSSGNMKDYAESVKGRFTISRDNVKNSLQLQMNSLRVEDTAVYYCARDEFPYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 403 - protein, 1142 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 404 - protein, 1141 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTFSNYWMNWVRQAPGKGLEWVANIKEDGGKKLYVDSVKGRFTISRDNAKNSLFLQMNSLRAEDTAVYYCAREDTTLVVDYYYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 405 - protein, 1135 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 406 - protein, 1137 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGSVVRPGGSLRLSCEASGFTFDDYGMSWVRQDPGKGLEWVSGINWNGDRTNYADSVKGRFIISRDNAKNSVYLQMNSLRAEDSALYHCARDQGLGVAATLDYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 407 - protein, 1144 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTVSNYEMNWVRQAPGKGLEWVSYISSSTSNIYYADSVKGRFTISRDNAENSLYLQMNSLRVEDTAVYYCVRDGIVVVPVGRGYYYYGLDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 408 - protein, 1137 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 409 - protein, 1135 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSEVQLVESGGGLVQPGRSLTLSCAASGFTFDNFGMHWVRQGPGKGLEWVSGLTWNSGVIGYADSVKGRFTISRDNAKNSLYLQMNSLRPEDTALYYCAKDIRNYGPFDYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 410 - protein, 1138 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSQVQLVQSGAEVKKPGSSVKVSCKASGGTFNTYAITWVRQAPGQGLEWMGGIIPISGIAEYAQKFQGRVTITTDDSSTTAYMELNSLRSEDTAVYYCASWNYALYYFYGMDVWGRGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 411 - protein, 1136 aa, synthetic construct
DIQMTQSPSSLSASIGDRVTITCRASQGISNYLAWYQQKPGKVPKLLIYAASTLQSGVPSRFSGSGSGTDFTLTISSLQPEDVATYYCQNHNSVPLTFGGGTKVEIKGGGGSGGGGSGGGGSEVQLVESGGGLVQPGGSLRLSCAASGFTFNSYAMTWVRQAPGKGLEWVSFIGGSTGNTYYAGSVKGRFTISSDNSKKTLYLQMNSLRAEDTAVYYCAKGGAARRMEYFQHWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 412 - protein, 1136 aa, synthetic construct
DIQMTQSPSSLSASVGDRVTITCRASQGISNYLAWYQQKPGKVPNLLIYAASTLQSGVPSRFSGSGSGTDFTLTISSLQPEDVATYYCQKYNSAPLTFGGGTKVEIKGGGGSGGGGSGGGGSEVQLVESGGGLVQPGGSLRLSCAASGFAFSSYAMTWVRQAPGKGLEWVSVISGTGGSTYYADSVKGRFTISRDNSKNTLYLQMNSLRAEDTAVYYCAKGGAARRMEYFQYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 413 - protein, 1143 aa, synthetic construct
DIVMTQSPLSLPVTPGEPASISCRSSQSLLHSNGYNYLDWYLQKPGQSPQFLIYLGSNRASGVPDRFSGSGSGTDFTLKINRVEAEDVGVYYCMQALQTPYTFGQGTKLEIKGGGGSGGGGSGGGGSQVQLVESGGGLVKPGGSLRLSCAASGFTFSVYYMNWIRQAPGKGLEWVSYISSSGSTIYYADSVKGRFTISRDNAKNSLYLQMNSLRAEDTAVYYCGREGYSGTYSYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 414 - protein, 1137 aa, synthetic construct
DIQMTQSPSSLSASVGDRVTITCRASQDISHYSAWYQQKPGKLPNLLIYAASTLQSGVPSRFSGSGSGTDFSLTTSSLQPEDVATYYCQKYNSVPLTFGGGTKVEIKGGGGSGGGGSGGGGSEVQLVESGGGLVQPGRSLRLSCAVSGFTFDDYAMHWVRQAPGKGLEWVSGISWNSGTRGYADSVKGRFTISRDNAKNSLYLQMNSLRGEDTALYYCVKDITISPNYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 415 - protein, 1132 aa, synthetic construct
DIQMTQSPSSLSASVGDRVTITCRASQSIDRYLNWYRQKPGKAPKLLIYTTSSLQSGVPSRFSGSGSGTDFTLTLSSLQPEDFATYYCQQSYSPPLTFGGGTKVEIKGGGGSGGGGSGGGGSQVQLVESGGGVVQPGRSLRLSCAASGFTFSSYGMHWVRQAPGKGLEWVAVIWYDGSNKYYADSVKGRFTISRDISKNTLYLQMNSLRAEDTAVYYCAGQLDLFFDYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 416 - protein, 1140 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKADSLPYAFGQGTKLEIKGGGGSGGGGSGGGGSEVQLVESGGGLVQPGGSLRLSCTASGFTFSNYWMTWVRQAPGKGLEWVANIKEDGSEKEYVDSVKGRFTISRDNAKNSLYLQMNSLRGEDTAVYYCARDGEQLVDYYYYYVMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 417 - protein, 1142 aa, synthetic construct
DIQMTQSPSSLSASVGDRVTITCRASQSISSYLNWYQQKPGKAPKVLIYAASSLQSGVPSRFSGSGSGTDFTLTISSLQPEDFATYYCQQSYSIPLTFGGGTKVEIKGGGGSGGGGSGGGGSEVQLVESGGGLVQPGRSLRLSCAASGFTFDDYAMHWVRQAPGKGLEWVSGISWNSGYIGYADSVKGRFTISRDNAENSLHLQMNSLRAEDTALYYCARGGSTLVRGVKGGYYGMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 418 - protein, 1131 aa, synthetic construct
EIVMTQSPATLSVSPGERATLSCRASQSVSSNFAWYQQKPGQAPRLLIYSASSRATGIPVRFSGSGSGTEFTLTISSLQSEDFAVYYCQQYNIWPRTFGQGTKVEIKGGGGSGGGGSGGGGSEVQLVESGGGLVQPGGSLRLSCAVSGFIFSSYEMNWVRQAPGKGLEWVSYISSSGSTIFYADSVKGRFTISRDNAKNSLYLQMNSLRAEDTAVYYCVSGVVLFDVWGQGTMVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 419 - protein, 1140 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKANSFPYTFGQGTKLEIKGGGGSGGGGSGGGGSEVQLVESGGGLVQPGGSLRLSCAASGFTFSNYWMTWVRQAPGKGLEWVANIKEDGSEKDYVDSVKGRFTISRDNAKNSLYLQMNSLRGEDTAVYYCARDGEQLVDYYYYYVMDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 420 - protein, 1135 aa, synthetic construct
DIQLTQSPSSLSASVGDRVTITCWASQGISSYLAWYQQKPGKAPKLLIYAASTLQSGVPSRFSGSGSGTEFTLTISSLQPEDFATYYCQQLNSYPLTFGGGTKVEIKGGGGSGGGGSGGGGSQVQLVESGGGVVQPGRSLRLSCIASGFTFSVYGIHWVRQAPGKGLEWMAVISHDGNIKHYADSVKGRFTISRDNSKNTLYLQINSLRTEDTAVYYCAKDTWNSLDTFDIWGQGTMVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 421 - protein, 1135 aa, synthetic construct
AIQMTQSPSSLSASVGDRVTITCRASQGIRNDLGWYQQKPGKAPKLLIYAASTLQSGVPSRFSGSGSGTDFTLTISSLQPEDFATYYCLQDYNYPFTFGPGTKVDIKGGGGSGGGGSGGGGSQVTLRESGPALVKPSQTLTLTCTFSGFSLNTYGMFVSWIRQPPGKALEWLAHIHWDDDKYYSTSLKTRLTISKDTSKNQVVLTMTNMDPVDTATYYCARGHNNLNYIIHWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 422 - protein, 1138 aa, synthetic construct
EIVLTQSPATLSLSPGERATLSCRASQSVSSYLAWYQQKPGQAPRLLIHDVSNRATGIPARFSGSGSGTDFTLTISSLEPEDFVVYYCQQRSDWPITFGQGTRLEIKGGGGSGGGGSGGGGSEVQLVESGGGLVQPGRSLRLSCAASGFTFDDKAMHWVRQVPGKGLEWISGISWNSGTIGYADSVKGRFIISRDNAKNSLYLQMNSLRAEDTALYYCAKDGDTSGWYWYGLDVWGQGTTVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 423 - protein, 1137 aa, synthetic construct
DIQMTQSPSSLSASVGDRVTITCRASQSIRNVLGWFQQKPGKAPQRLIYAASSLQSGVPSRFSGSGSGTEFTLTISSLQPEDFATYYCLQHNFYPLTFGGGTKVEIKGGGGSGGGGSGGGGSQVQLVESGGGVVQPGRSLRLSCAASGFTFTTYGMHWVRQAPGKGLEWVAVIWYDGSNKYYGDSVKGRFTISRDNSKNTLYLQMNSLRVDDTAVYYCTRTHGYTRSSDGFDYWGQGTLVTVSSGGGGSGGGGSAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 424 - protein, 107 aa, synthetic construct [sequence omitted]

SEQ ID NO: 425 - protein, 107 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTH

SEQ ID NO: 426 - protein, 5 aa, synthetic construct
GGGGS

SEQ ID NO: 427 - protein, 243 aa, synthetic construct
DIQMTQSPSSLSASVGDRVTITCRASQGIRDHFGWYQQKPGKAPKRLIYAASSLHSGVPSRFSGSGSGTEFTLTISSLQPEDFATYYCLQYDTYPLTFGGGTKVEIKGGGGSGGGGSGGGGSEVQLVESGGGLVQPGGSLRLSCATSGFTFTSYDMKWVRQAPGLGLEWVSAISGSGGNTYYADSVKGRFTISRDNSRNTLYLQMNSLRAEDTAVYYCTRSHDFGAFDYFDYWGQGTLVTVSS

SEQ ID NO: 428 - protein, 245 aa, synthetic construct
EIVMTQSPATLSVSPGERATLSCRASQTVSSNLAWYQQKPGQAPRLLIYGSSSRATGIPARFSGSGSGTEFTLTISSLQSEDFAVYYCQQYNNWPPYTFGQGTKLEIKGGGGSGGGGSGGGGSEVQLVESGGDLVQPGRSLRLSCAASGFTFDDYAMHWVRQAPGKGLEWVSGISWNSATRVYADSVKGRFTISRDNAKNFLYLQMNSLRSEDTALYHCAKDMDISLGYYGLDVWGQGTTVTVSS

SEQ ID NO: 429 - protein, 246 aa, synthetic construct
DIQMTQSPSSVSASVGDRVTITCRASQGIASWLAWYQQKPGKAPELLIYAASSLQGGVPSRFSGSGSGTDFTLTISSLQPEDFAIYYCQQANYFPWTFGQGTKVEIKGGGGSGGGGSGGGGSQITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSS

SEQ ID NO: 430 - protein, 243 aa, synthetic construct
DIQMTQSPSSLSASVGDRVTITCRASQGIRTDLGWYQQKPGKAPKRLIYAASSLQSGVPSRFSGSGSGTEFTLTISSLRPEDFATFYCLQYNSYPLTFGGGTKVEIKGGGGSGGGGSGGGGSEVQLLESGGALVQPGGSLRLSCAASGFTFTSYAMHWVRQAPGKGLEWVSSIRGSGGGTYSADSVKGRFTISRDNSRDTLYLQMNSVRAEDTAVYYCARSHDYGAFDFFDYWGQGTLVTVSS

SEQ ID NO: 431 - protein, 238 aa, synthetic construct
EIVMTQSPATLSVSPGERATLSCRASQSVSINLAWYQQKPGQAPRLLIFVASTRATGIPARFSGSGSGTEFTLTISSLQSEDFATYYCQQYDIWPYTFGQGTKLEIKGGGGSGGGGSGGGGSQVQLVESGGGLVKPGGSLRLSCAASGFTFSDYFMSWIRQAPGKGLEWVSYISSTGSTINYADSVKGRFTISRDNVKNSLYLQMTSLRVEDTAVYYCTRDNWNYEYWGQGTLVTVSS

SEQ ID NO: 432 - protein, 245 aa, synthetic construct
DIQMTQSPSSLSASVGDRVTINCRASQGIRNDLGWYQQKPGKAPKRLIYAASSLQSGVPLRFSGSGSGTEFTLTINNLQPEDFATYYCLSHNSYPWTFGQGTKVEIKGGGGSGGGGSGGGGSQLQLQESGPGLVKPSETLSLTCTVSGESISSNTYYWGWIRQPPGKGLEWIGSIDYSGTTNYNPSLKSRVTISVDTSRNHFSLRLRSVTAADTAVYYCAREWGNYGYYYGMDVWGQGTTVTVSS

SEQ ID NO: 433 - protein, 240 aa, synthetic construct
DIQMTQSPPSVSASVGDRVTITCRASQGISSWLAWYQQKPGKAPKLLIYAASSLQSGVPSRFSGSGSGTDFTLTISSLQPEDFATYYCQQANSFPRTFGQGTKVEIKGGGGSGGGGSGGGGSQVQLVQSGAEVKKPGSSVRVSCKASRGTFSSYAISWVRQAPGQGLEWMGGIIPIFGTANYAQKFLARVTITADESTSTAYMELSSLRSEDTAVYYCAREKGWNYFDYWGQGTLVTVSS

SEQ ID NO: 434 - protein, 250 aa, synthetic construct
DIVMTQSPLSLPVTPGEPASISCRSSQSLLHGNGYNYLTWYLQKPGQSPQLLIYLGSNRASGVPDRFSGSGSGTDFTLKISRVEAEDVGVYYCMQALQTPYTFGQGTKLEIKGGGGSGGGGSGGGGSQVQLVESGGGLVKPGGSLRLSCAASGFTFSDYYMNWIRQAPGKGLEWVSYISSSGTTIYYADSVKGRFTISRDNAKKSLYLEMNSLRAEDTAVYYCAREGYGNDYYYYGIDVWGQGTTVTVSS

SEQ ID NO: 435 - protein, 245 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVESGGGVVQPGRSLRLSCAASGFTFSSFGMHWVRQAPGKGLEWVIFISYDGSDKYYADSVKGRFAISRDSSKNTLYLQMNSLRAEDTAVYYCAKENGILTDSYGMDVWGQGTTVTVSS

SEQ ID NO: 436 - protein, 247 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVQSGAEVKKPGASVKVSCKASGYTFTSYGISWVRQAPGQGLEWMGWISVYHGNTNYAQKFQGRVTMTTDTSTSTAYMELRSLRSDDTAVYYCAREGYYDFWSGYYPFDYWGQGTLVTVSS

SEQ ID NO: 437 - protein, 241 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLIQPGGSLRLSCEASGFTFRNYEMNWVRQAPGKGLEWVSYISSSGNMKDYAESVKGRFTISRDNVKNSLQLQMNSLRVEDTAVYYCARDEFPYGMDVWGQGTTVTVSS

SEQ ID NO: 438 - protein, 249 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSQVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSS

SEQ ID NO: 439 - protein, 248 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTFSNYWMNWVRQAPGKGLEWVANIKEDGGKKLYVDSVKGRFTISRDNAKNSLFLQMNSLRAEDTAVYYCAREDTTLVVDYYYYGMDVWGQGTTVTVSS

SEQ ID NO: 440 - protein, 242 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSS

SEQ ID NO: 441 - protein, 244 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGSVVRPGGSLRLSCEASGFTFDDYGMSWVRQDPGKGLEWVSGINWNGDRTNYADSVKGRFIISRDNAKNSVYLQMNSLRAEDSALYHCARDQGLGVAATLDYWGQGTLVTVSS

SEQ ID NO: 442 - protein, 251 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGGSLRLSCAASGFTVSNYEMNWVRQAPGKGLEWVSYISSSTSNIYYADSVKGRFTISRDNAENSLYLQMNSLRVEDTAVYYCVRDGIVVVPVGRGYYYYGLDVWGQGTTVTVSS

SEQ ID NO: 443 - protein, 244 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKGGGGSGGGGSGGGGSEVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSS

SEQ ID NO: 444 - protein, 242 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSEVQLVESGGGLVQPGRSLTLSCAASGFTFDNFGMHWVRQGPGKGLEWVSGLTWNSGVIGYADSVKGRFTISRDNAKNSLYLQMNSLRPEDTALYYCAKDIRNYGPFDYWGQGTLVTVSS

SEQ ID NO: 445 - protein, 245 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKGGGGSGGGGSGGGGSQVQLVQSGAEVKKPGSSVKVSCKASGGTFNTYAITWVRQAPGQGLEWMGGIIPISGIAEYAQKFQGRVTITTDDSSTTAYMELNSLRSEDTAVYYCASWNYALYYFYGMDVWGRGTTVTVSS

SEQ ID NO: 446 - protein, 243 aa, synthetic construct
DIQMTQSPSSLSASIGDRVTITCRASQGISNYLAWYQQKPGKVPKLLIYAASTLQSGVPSRFSGSGSGTDFTLTISSLQPEDVATYYCQNHNSVPLTFGGGTKVEIKGGGGSGGGGSGGGGSEVQLVESGGGLVQPGGSLRLSCAASGFTFNSYAMTWVRQAPGKGLEWVSFIGGSTGNTYYAGSVKGRFTISSDNSKKTLYLQMNSLRAEDTAVYYCAKGGAARRMEYFQHWGQGTLVTVSS

SEQ ID NO: 447 - protein, 243 aa, synthetic construct
DIQMTQSPSSLSASVGDRVTITCRASQGISNYLAWYQQKPGKVPNLLIYAASTLQSGVPSRFSGSGSGTDFTLTISSLQPEDVATYYCQKYNSAPLTFGGGTKVEIKGGGGSGGGGSGGGGSEVQLVESGGGLVQPGGSLRLSCAASGFAFSSYAMTWVRQAPGKGLEWVSVISGTGGSTYYADSVKGRFTISRDNSKNTLYLQMNSLRAEDTAVYYCAKGGAARRMEYFQYWGQGTLVTVSS

SEQ ID NO: 448 - protein, 250 aa, synthetic construct
DIVMTQSPLSLPVTPGEPASISCRSSQSLLHSNGYNYLDWYLQKPGQSPQFLIYLGSNRASGVPDRFSGSGSGTDFTLKINRVEAEDVGVYYCMQALQTPYTFGQGTKLEIKGGGGSGGGGSGGGGSQVQLVESGGGLVKPGGSLRLSCAASGFTFSVYYMNWIRQAPGKGLEWVSYISSSGSTIYYADSVKGRFTISRDNAKNSLYLQMNSLRAEDTAVYYCGREGYSGTYSYYGMDVWGQGTTVTVSS

SEQ ID NO: 449 - protein, 244 aa, synthetic construct
DIQMTQSPSSLSASVGDRVTITCRASQDISHYSAWYQQKPGKLPNLLIYAASTLQSGVPSRFSGSGSGTDFSLTTSSLQPEDVATYYCQKYNSVPLTFGGGTKVEIKGGGGSGGGGSGGGGSEVQLVESGGGLVQPGRSLRLSCAVSGFTFDDYAMHWVRQAPGKGLEWVSGISWNSGTRGYADSVKGRFTISRDNAKNSLYLQMNSLRGEDTALYYCVKDITISPNYYGMDVWGQGTTVTVSS

SEQ ID NO: 450 - protein, 239 aa, synthetic construct
DIQMTQSPSSLSASVGDRVTITCRASQSIDRYLNWYRQKPGKAPKLLIYTTSSLQSGVPSRFSGSGSGTDFTLTLSSLQPEDFATYYCQQSYSPPLTFGGGTKVEIKGGGGSGGGGSGGGGSQVQLVESGGGVVQPGRSLRLSCAASGFTFSSYGMHWVRQAPGKGLEWVAVIWYDGSNKYYADSVKGRFTISRDISKNTLYLQMNSLRAEDTAVYYCAGQLDLFFDYWGQGTLVTVSS

SEQ ID NO: 451 - protein, 247 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKADSLPYAFGQGTKLEIKGGGGSGGGGSGGGGSEVQLVESGGGLVQPGGSLRLSCTASGFTFSNYWMTWVRQAPGKGLEWVANIKEDGSEKEYVDSVKGRFTISRDNAKNSLYLQMNSLRGEDTAVYYCARDGEQLVDYYYYYVMDVWGQGTTVTVSS

SEQ ID NO: 452 - protein, 249 aa, synthetic construct
DIQMTQSPSSLSASVGDRVTITCRASQSISSYLNWYQQKPGKAPKVLIYAASSLQSGVPSRFSGSGSGTDFTLTISSLQPEDFATYYCQQSYSIPLTFGGGTKVEIKGGGGSGGGGSGGGGSEVQLVESGGGLVQPGRSLRLSCAASGFTFDDYAMHWVRQAPGKGLEWVSGISWNSGYIGYADSVKGRFTISRDNAENSLHLQMNSLRAEDTALYYCARGGSTLVRGVKGGYYGMDVWGQGTTVTVSS

SEQ ID NO: 453 - protein, 238 aa, synthetic construct
EIVMTQSPATLSVSPGERATLSCRASQSVSSNFAWYQQKPGQAPRLLIYSASSRATGIPVRFSGSGSGTEFTLTISSLQSEDFAVYYCQQYNIWPRTFGQGTKVEIKGGGGSGGGGSGGGGSEVQLVESGGGLVQPGGSLRLSCAVSGFIFSSYEMNWVRQAPGKGLEWVSYISSSGSTIFYADSVKGRFTISRDNAKNSLYLQMNSLRAEDTAVYYCVSGVVLFDVWGQGTMVTVSS

SEQ ID NO: 454 - protein, 247 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKANSFPYTFGQGTKLEIKGGGGSGGGGSGGGGSEVQLVESGGGLVQPGGSLRLSCAASGFTFSNYWMTWVRQAPGKGLEWVANIKEDGSEKDYVDSVKGRFTISRDNAKNSLYLQMNSLRGEDTAVYYCARDGEQLVDYYYYYVMDVWGQGTTVTVSS

SEQ ID NO: 455 - protein, 242 aa, synthetic construct
DIQLTQSPSSLSASVGDRVTITCWASQGISSYLAWYQQKPGKAPKLLIYAASTLQSGVPSRFSGSGSGTEFTLTISSLQPEDFATYYCQQLNSYPLTFGGGTKVEIKGGGGSGGGGSGGGGSQVQLVESGGGVVQPGRSLRLSCIASGFTFSVYGIHWVRQAPGKGLEWMAVISHDGNIKHYADSVKGRFTISRDNSKNTLYLQINSLRTEDTAVYYCAKDTWNSLDTFDIWGQGTMVTVSS

SEQ ID NO: 456 - protein, 242 aa, synthetic construct
AIQMTQSPSSLSASVGDRVTITCRASQGIRNDLGWYQQKPGKAPKLLIYAASTLQSGVPSRFSGSGSGTDFTLTISSLQPEDFATYYCLQDYNYPFTFGPGTKVDIKGGGGSGGGGSGGGGSQVTLRESGPALVKPSQTLTLTCTFSGFSLNTYGMFVSWIRQPPGKALEWLAHIHWDDDKYYSTSLKTRLTISKDTSKNQVVLTMTNMDPVDTATYYCARGHNNLNYIIHWGQGTLVTVSS

SEQ ID NO: 457 - protein, 245 aa, synthetic construct
EIVLTQSPATLSLSPGERATLSCRASQSVSSYLAWYQQKPGQAPRLLIHDVSNRATGIPARFSGSGSGTDFTLTISSLEPEDFVVYYCQQRSDWPITFGQGTRLEIKGGGGSGGGGSGGGGSEVQLVESGGGLVQPGRSLRLSCAASGFTFDDKAMHWVRQVPGKGLEWISGISWNSGTIGYADSVKGRFIISRDNAKNSLYLQMNSLRAEDTALYYCAKDGDTSGWYWYGLDVWGQGTTVTVSS

SEQ ID NO: 458 - protein, 244 aa, synthetic construct
DIQMTQSPSSLSASVGDRVTITCRASQSIRNVLGWFQQKPGKAPQRLIYAASSLQSGVPSRFSGSGSGTEFTLTISSLQPEDFATYYCLQHNFYPLTFGGGTKVEIKGGGGSGGGGSGGGGSQVQLVESGGGVVQPGRSLRLSCAASGFTFTTYGMHWVRQAPGKGLEWVAVIWYDGSNKYYGDSVKGRFTISRDNSKNTLYLQMNSLRVDDTAVYYCTRTHGYTRSSDGFDYWGQGTLVTVSS

SEQ ID NO: 459 - protein, 111 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPLLQGSG

SEQ ID NO: 460 - protein, 700 aa, synthetic construct
HHHHHHEQKLISEEDLGGEQKLISEEDLCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 461 - protein, 28 aa, synthetic construct
EQKLISEEDLGGEQKLISEEDLHHHHHH

SEQ ID NO: 462 - protein, 121 aa, synthetic construct
EVQLVESGGGLVQPGGSLRLSCAASGFAFSSYAMTWVRQAPGKGLEWVSVISGTGGSTYYADSVKGRFTISRDNSKNTLYLQMNSLRAEDTAVYYCAKGGAARRMEYFQYWGQGTTVTVSS

SEQ ID NO: 463 - protein, 121 aa, synthetic construct
EVQLVESGGGLVQPGGSLRLSCAASGFTFNSYAMTWVRQAPGKGLEWVSFIGGSTGNTYYAGSVKGRFTISSDNSKKTLYLQMNSLRAEDTAVYYCAKGGAARRMEYFQHWGQGTTVTVSS

SEQ ID NO: 464 - protein, 122 aa, synthetic construct
EVQLVESGGGVVQPGRSLRLSCAASGFTFTTYGMHWVRQAPGKGLEWVAVIWYDGSNKYYGDSVKGRFTISRDNSKNTLYLQMNSLRVDDTAVYYCTRTHGYTRSSDGFDYWGQGTMVTVSS

SEQ ID NO: 465 - protein, 107 aa, synthetic construct
DIVMTQSPATLSLSPGERATLSCRASQSVSSYLAWYQQKPGQAPRLLIHDVSNRATGIPARFSGSGSGTDFTLTISSLEPEDFVVYYCQQRSDWPITFGQGTRLEIK

SEQ ID NO: 466 - protein, 107 aa, synthetic construct
DIQMTQSPSSLSASVGDRVTITCWASQGISSYLAWYQQKPGKAPKLLIYAASTLQSGVPSRFSGSGSGTEFTLTISSLQPEDFATYYCQQLNSYPLTFGGGTKVEIK

SEQ ID NO: 467 - protein, 120 aa, synthetic construct
QVQLVESGPALVKPSQTLTLTCTFSGFSLNTYGMFVSWIRQPPGKALEWLAHIHWDDDKYYSTSLKTRLTISKDTSKNQVVLTMTNMDPVDTATYYCARGHNNLNYIIHWGQGTLVTVSS

SEQ ID NO: 468 - protein, 107 aa, synthetic construct
DILMTQSPSSLSASVGDRVTITCRASQGIRNDLGWYQQKPGKAPKLLIYAASTLQSGVPSRFSGSGSGTDFTLTISSLQPEDFATYYCLQDYNYPFTFGPGTKVEIK

SEQ ID NO: 469 - protein, 107 aa, synthetic construct
DIVMTQSPATLSVSPGERATLSCRASQSVSSNFAWYQQKPGQAPRLLIYSASSRATGIPVRFSGSGSGTEFTLTISSLQSEDFAVYYCQQYNIWPRTFGQGTKVEIK

SEQ ID NO: 470 - protein, 125 aa, synthetic construct
QVQLVESGGGLVQPGGSLRLSCAASGFTFSNYWMTWVRQAPGKGLEWVANIKEDGSEKDYVDSVKGRFTISRDNAKNSLYLQMNSLRGEDTAVYYCARDGEQLVDYYYYYVMDVWGQGTTVTVSS

SEQ ID NO: 471 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQKANSFPYTFGQGTKVEIK

SEQ ID NO: 472 - protein, 117 aa, synthetic construct
EVQLVESGGGVVQPGRSLRLSCAASGFTFSSYGMHWVRQAPGKGLEWVAVIWYDGSNKYYADSVKGRFTISRDISKNTLYLQMNSLRAEDTAVYYCAGQLDLFFDYWGQGTLVTVSS

SEQ ID NO: 473 - protein, 121 aa, synthetic construct
EVQLVQSGGGLVQPGGSLRLSCATSGFTFTSYDMKWVRQAPGLGLEWVSAISGSGGNTYYADSVKGRFTISRDNSRNTLYLQMNSLRAEDTAVYYCTRSHDFGAFDYFDYWGQGTMVTVSS

SEQ ID NO: 474 - protein, 107 aa, synthetic construct
DIQLTQSPSSLSASVGDRVTITCRASQGIRDHFGWYQQKPGKAPKRLIYAASSLHSGVPSRFSGSGSGTEFTLTISSLQPEDFATYYCLQYDTYPLTFGGGTKVEIK

SEQ ID NO: 475 - protein, 121 aa, synthetic construct
EVQLLESGGALVQPGGSLRLSCAASGFTFTSYAMHWVRQAPGKGLEWVSSIRGSGGGTYSADSVKGRFTISRDNSRDTLYLQMNSVRAEDTAVYYCARSHDYGAFDFFDYWGQGTTVTVSS

SEQ ID NO: 476 - protein, 107 aa, synthetic construct
DIQMTQSPSSLSASVGDRVTITCRASQGIRTDLGWYQQKPGKAPKRLIYAASSLQSGVPSRFSGSGSGTEFTLTISSLRPEDFATFYCLQYNSYPLTFGGGTKVDIK

SEQ ID NO: 477 - protein, 123 aa, synthetic construct
QVQLVESGPGLVKPSETLSLTCTVSGESISSNTYYWGWIRQPPGKGLEWIGSIDYSGTTNYNPSLKSRVTISVDTSRNHFSLRLRSVTAADTAVYYCAREWGNYGYYYGMDVWGQGTTVTVSS

SEQ ID NO: 478 - protein, 123 aa, synthetic construct
EVQLVESGGGLVKPGGSLRLSCAASGFTFSDYYMNWIRQAPGKGLEWVSYISSSGTTIYYADSVKGRFTISRDNAKKSLYLEMNSLRAEDTAVYYCAREGYGNDYYYYGIDVWGQGTTVTVSS

SEQ ID NO: 479 - protein, 112 aa, synthetic construct
DIQLTQSPLSLPVTPGEPASISCRSSQSLLHGNGYNYLTWYLQKPGQSPQLLIYLGSNRASGVPDRFSGSGSGTDFTLKISRVEAEDVGVYYCMQALQTPYTFGQGTKVEIK

SEQ ID NO: 480 - protein, 122 aa, synthetic construct
EVQLVESGGGVVQPGRSLRLSCAASGFTFSSFGMHWVRQAPGKGLEWVIFISYDGSDKYYADSVKGRFAISRDSSKNTLYLQMNSLRAEDTAVYYCAKENGILTDSYGMDVWGQGTTVTVSS

SEQ ID NO: 481 - protein, 124 aa, synthetic construct
EVQLVESGAEVKKPGASVKVSCKASGYTFTSYGISWVRQAPGQGLEWMGWISVYHGNTNYAQKFQGRVTMTTDTSTSTAYMELRSLRSDDTAVYYCAREGYYDFWSGYYPFDYWGQGTTVTVSS

SEQ ID NO: 482 - protein, 121 aa, synthetic construct
EVQLVESGGSVVRPGGSLRLSCEASGFTFDDYGMSWVRQDPGKGLEWVSGINWNGDRTNYADSVKGRFIISRDNAKNSVYLQMNSLRAEDSALYHCARDQGLGVAATLDYWGQGTMVTVSS

SEQ ID NO: 483 - protein, 123 aa, synthetic construct
EVQLVESGGGLVKPGGSLRLSCAASGFTFSVYYMNWIRQAPGKGLEWVSYISSSGSTIYYADSVKGRFTISRDNAKNSLYLQMNSLRAEDTAVYYCGREGYSGTYSYYGMDVWGQGTTVTVSS

SEQ ID NO: 484 - protein, 107 aa, synthetic construct
DIQLTQSPSSLSASVGDRVTITCRASQDISHYSAWYQQKPGKLPNLLIYAASTLQSGVPSRFSGSGSGTDFSLTTSSLQPEDVATYYCQKYNSVPLTFGGRTKVEIK

SEQ ID NO: 485 - protein, 233 aa, synthetic construct
EVQLVESGGDLVQPGRSLRLSCAASGFTFDDYAMHWVRQAPGKGLEWVSGISWNSATRVYADSVKGRFTISRDNAKNFLYLQMNSLRSEDTALYHCAKDMDISLGYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPLLQGSG

SEQ ID NO: 486 - protein, 235 aa, synthetic construct
QITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPLLQGSG

SEQ ID NO: 487 - protein, 232 aa, synthetic construct
EVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPLLQGSG

SEQ ID NO: 488 - protein, 112 aa, synthetic construct
DIQLTQSPLSLPVTPGEPASISCRSSQSLLHSNGYNYLDWYLQKPGQSPQFLIYLGSNRASGVPDRFSGSGSGTDFTLKINRVEAEDVGVYYCMQALQTPYTFGQGTKVEIK

SEQ ID NO: 489 - protein, 230 aa, synthetic construct
EVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPLLQGSG

SEQ ID NO: 490 - protein, 239 aa, synthetic construct
EVQLVESGGGLVQPGGSLRLSCAASGFTVSNYEMNWVRQAPGKGLEWVSYISSSTSNIYYADSVKGRFTISRDNAENSLYLQMNSLRVEDTAVYYCVRDGIVVVPVGRGYYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPLLQGSG

SEQ ID NO: 491 - protein, 237 aa, synthetic construct
QVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPLLQGSG

SEQ ID NO: 492 - protein, 116 aa, synthetic construct
QVQLVESGGGLVQPGGSLRLSCAVSGFIFSSYEMNWVRQAPGKGLEWVSYISSSGSTIFYADSVKGRFTISRDNAKNSLYLQMNSLRAEDTAVYYCVSGVVLFDVWGQGTMVTVSS

SEQ ID NO: 493 - protein, 105 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPP

SEQ ID NO: 494 - protein, 227 aa, synthetic construct
EVQLVESGGDLVQPGRSLRLSCAASGFTFDDYAMHWVRQAPGKGLEWVSGISWNSATRVYADSVKGRFTISRDNAKNFLYLQMNSLRSEDTALYHCAKDMDISLGYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPP

SEQ ID NO: 495 - protein, 229 aa, synthetic construct
QITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPP

SEQ ID NO: 496 - protein, 226 aa, synthetic construct
EVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPP

SEQ ID NO: 497 - protein, 224 aa, synthetic construct
EVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPP

SEQ ID NO: 498 - protein, 233 aa, synthetic construct
EVQLVESGGGLVQPGGSLRLSCAASGFTVSNYEMNWVRQAPGKGLEWVSYISSSTSNIYYADSVKGRFTISRDNAENSLYLQMNSLRVEDTAVYYCVRDGIVVVPVGRGYYYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPP

SEQ ID NO: 499 - protein, 231 aa, synthetic construct
QVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPP

SEQ ID NO: 500 - protein, 29 aa, synthetic construct
MHRPRRRGTRPPPLALLAALLLAARGADA

SEQ ID NO: 501 - protein, 6 aa, synthetic construct
LLQGSG

SEQ ID NO: 502 - protein, 6 aa, synthetic construct
HHHHHH

SEQ ID NO: 503 - DNA, 20 bp, synthetic construct
  source 1..20: mol_type = other DNA
gctgcattgcggactgtaga

SEQ ID NO: 504 - DNA, 23 bp, synthetic construct
  source 1..23: mol_type = other DNA
tccatcattctcagctgctacaa

SEQ ID NO: 505 - protein, 22 aa, synthetic construct
LLNENSYVPREAGSQKDENLAL

SEQ ID NO: 506 - protein, 15 aa, synthetic construct
IYMDQTKFPIVNAEL

SEQ ID NO: 507 - protein, 5 aa, synthetic construct
TYKEL

SEQ ID NO: 508 - protein, 19 aa, synthetic construct
KRKLSEKLDSTDFTGTIKL

SEQ ID NO: 509 - protein, 20 aa, synthetic construct
YTLIEKTMQNVKHPVTGQFL

SEQ ID NO: 510 - protein, 17 aa, synthetic construct
LIERIPELNKVARAAAE

SEQ ID NO: 511 - protein, 19 aa, synthetic construct
LNENSYVPREAGSQKDENL

SEQ ID NO: 512 - protein, 9 aa, synthetic construct
GTKKDFEDL

SEQ ID NO: 513 - protein, 26 aa, synthetic construct
SVIIVDKNGRLVYLVENPGGYVAYSK

SEQ ID NO: 514 - protein, 19 aa, synthetic construct
LLNENSYVPREAGSQKDEN

SEQ ID NO: 515 - protein, 12 aa, synthetic construct
DQTKFPIVNAEL

SEQ ID NO: 516 - protein, 14 aa, synthetic construct
TYKELIERIPELNK

SEQ ID NO: 517 - protein, 24 aa, synthetic construct
SVIIVDKNGRLVYLVENPGGYVAY

SEQ ID NO: 518 - protein, 20 aa, synthetic construct
FGNMEGDCPSDWKTDSTCRM

SEQ ID NO: 519 - protein, 4 aa, synthetic construct
FEDL

SEQ ID NO: 520 - protein, 21 aa, synthetic construct
LVENPGGYVAYSKAATVTGKL

SEQ ID NO: 521 - protein, 17 aa, synthetic construct
IYMDQTKFPIVNAELSF

SEQ ID NO: 522 - protein, 9 aa, synthetic construct
ISRAAAEKL

SEQ ID NO: 523 - protein, 18 aa, synthetic construct
VTSESKNVKLTVSNVLKE

SEQ ID NO: 524 - protein, 16 aa, synthetic construct
FCEDTDYPYLGTTMDT

SEQ ID NO: 525 - protein, 4 aa, synthetic construct
LLNE

SEQ ID NO: 526 - protein, 8 aa, synthetic construct
DSTDFTGT

SEQ ID NO: 527 - protein, 9 aa, synthetic construct
VKHPVTGQF

SEQ ID NO: 528 - protein, 7 aa, synthetic construct
IERIPEL

SEQ ID NO: 529 - protein, 18 aa, synthetic construct
LNENSYVPREAGSQKDEN

SEQ ID NO: 530 - protein, 8 aa, synthetic construct
IVDKNGRL

SEQ ID NO: 531 - protein, 10 aa, synthetic construct
IVDKNGRLVY

SEQ ID NO: 532 - protein, 5 aa, synthetic construct
DQTKF

SEQ ID NO: 533 - protein, 8 aa, synthetic construct
LVENPGGY

SEQ ID NO: 534 - protein, 9 aa, synthetic construct
PIVNAELSF

SEQ ID NO: 535 - protein, 9 aa, synthetic construct
PYLGTTMDT

SEQ ID NO: 536 - protein, 220 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 537 - protein, 10 aa, synthetic construct
GGGGSGGGGS

SEQ ID NO: 538 - protein, 15 aa, synthetic construct
GGGGSGGGGSGGGGS

SEQ ID NO: 539 - protein, 451 aa, synthetic construct
EVQLVESGGGLVQPGGSLRLSCAASGFAFSSYAMTWVRQAPGKGLEWVSVISGTGGSTYYADSVKGRFTISRDNSKNTLYLQMNSLRAEDTAVYYCAKGGAARRMEYFQYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 540 - protein, 451 aa, synthetic construct
EVQLVESGGGLVQPGGSLRLSCAASGFTFNSYAMTWVRQAPGKGLEWVSFIGGSTGNTYYAGSVKGRFTISSDNSKKTLYLQMNSLRAEDTAVYYCAKGGAARRMEYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 541 - protein, 452 aa, synthetic construct
QVQLVESGGGVVQPGRSLRLSCAASGFTFTTYGMHWVRQAPGKGLEWVAVIWYDGSNKYYGDSVKGRFTISRDNSKNTLYLQMNSLRVDDTAVYYCTRTHGYTRSSDGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 542 - protein, 453 aa, synthetic construct
EVQLVESGGGLVQPGRSLRLSCAASGFTFDDKAMHWVRQVPGKGLEWISGISWNSGTIGYADSVKGRFIISRDNAKNSLYLQMNSLRAEDTALYYCAKDGDTSGWYWYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 543 - protein, 450 aa, synthetic construct
QVQLVESGGGVVQPGRSLRLSCIASGFTFSVYGIHWVRQAPGKGLEWMAVISHDGNIKHYADSVKGRFTISRDNSKNTLYLQINSLRTEDTAVYYCAKDTWNSLDTFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 544 - protein, 450 aa, synthetic construct
QVTLRESGPALVKPSQTLTLTCTFSGFSLNTYGMFVSWIRQPPGKALEWLAHIHWDDDKYYSTSLKTRLTISKDTSKNQVVLTMTNMDPVDTATYYCARGHNNLNYIIHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 545 - protein, 446 aa, synthetic construct
EVQLVESGGGLVQPGGSLRLSCAVSGFIFSSYEMNWVRQAPGKGLEWVSYISSSGSTIFYADSVKGRFTISRDNAKNSLYLQMNSLRAEDTAVYYCVSGVVLFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 546 - protein, 455 aa, synthetic construct
EVQLVESGGGLVQPGGSLRLSCAASGFTFSNYWMTWVRQAPGKGLEWVANIKEDGSEKDYVDSVKGRFTISRDNAKNSLYLQMNSLRGEDTAVYYCARDGEQLVDYYYYYVMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 547 - protein, 457 aa, synthetic construct
EVQLVESGGGLVQPGRSLRLSCAASGFTFDDYAMHWVRQAPGKGLEWVSGISWNSGYIGYADSVKGRFTISRDNAENSLHLQMNSLRAEDTALYYCARGGSTLVRGVKGGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 548 - protein, 447 aa, synthetic construct
QVQLVESGGGVVQPGRSLRLSCAASGFTFSSYGMHWVRQAPGKGLEWVAVIWYDGSNKYYADSVKGRFTISRDISKNTLYLQMNSLRAEDTAVYYCAGQLDLFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 549 - protein, 455 aa, synthetic construct
EVQLVESGGGLVQPGGSLRLSCTASGFTFSNYWMTWVRQAPGKGLEWVANIKEDGSEKEYVDSVKGRFTISRDNAKNSLYLQMNSLRGEDTAVYYCARDGEQLVDYYYYYVMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 550 - protein, 451 aa, synthetic construct
EVQLVESGGGLVQPGGSLRLSCATSGFTFTSYDMKWVRQAPGLGLEWVSAISGSGGNTYYADSVKGRFTISRDNSRNTLYLQMNSLRAEDTAVYYCTRSHDFGAFDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 551 - protein, 452 aa, synthetic construct
EVQLVESGGDLVQPGRSLRLSCAASGFTFDDYAMHWVRQAPGKGLEWVSGISWNSATRVYADSVKGRFTISRDNAKNFLYLQMNSLRSEDTALYHCAKDMDISLGYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 552 - protein, 454 aa, synthetic construct
QITLKESGPTLVKPTQTLTLTCTFSGFSLSTSGVGVVWIRQPPGKALEWLALIYWNDHKRYSPSLGSRLTITKDTSKNQVVLTMTNMDPVDTATYYCAHYSGSYSYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 553 - protein, 451 aa, synthetic construct
EVQLLESGGALVQPGGSLRLSCAASGFTFTSYAMHWVRQAPGKGLEWVSSIRGSGGGTYSADSVKGRFTISRDNSRDTLYLQMNSVRAEDTAVYYCARSHDYGAFDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 554 - protein, 446 aa, synthetic construct
QVQLVESGGGLVKPGGSLRLSCAASGFTFSDYFMSWIRQAPGKGLEWVSYISSTGSTINYADSVKGRFTISRDNVKNSLYLQMTSLRVEDTAVYYCTRDNWNYEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 555 - protein, 453 aa, synthetic construct
QLQLQESGPGLVKPSETLSLTCTVSGESISSNTYYWGWIRQPPGKGLEWIGSIDYSGTTNYNPSLKSRVTISVDTSRNHFSLRLRSVTAADTAVYYCAREWGNYGYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 556 - protein, 448 aa, synthetic construct
QVQLVQSGAEVKKPGSSVRVSCKASRGTFSSYAISWVRQAPGQGLEWMGGIIPIFGTANYAQKFLARVTITADESTSTAYMELSSLRSEDTAVYYCAREKGWNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 557 - protein, 453 aa, synthetic construct
QVQLVESGGGLVKPGGSLRLSCAASGFTFSDYYMNWIRQAPGKGLEWVSYISSSGTTIYYADSVKGRFTISRDNAKKSLYLEMNSLRAEDTAVYYCAREGYGNDYYYYGIDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 558 - protein, 452 aa, synthetic construct
QVQLVESGGGVVQPGRSLRLSCAASGFTFSSFGMHWVRQAPGKGLEWVIFISYDGSDKYYADSVKGRFAISRDSSKNTLYLQMNSLRAEDTAVYYCAKENGILTDSYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 559 - protein, 454 aa, synthetic construct
QVQLVQSGAEVKKPGASVKVSCKASGYTFTSYGISWVRQAPGQGLEWMGWISVYHGNTNYAQKFQGRVTMTTDTSTSTAYMELRSLRSDDTAVYYCAREGYYDFWSGYYPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 560 - protein, 448 aa, synthetic construct
EVQLVESGGGLIQPGGSLRLSCEASGFTFRNYEMNWVRQAPGKGLEWVSYISSSGNMKDYAESVKGRFTISRDNVKNSLQLQMNSLRVEDTAVYYCARDEFPYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 561 - protein, 451 aa, synthetic construct
EVQLVESGGGLVQPGRSLRLSCAASGFTFDDYAMNWVRQAPGKGLEWVSGISWSSGSMDYADSVKGRFTISRDNAKNSLYLQMNSLRTEDTALYYCAKAREVGDYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 562 - protein, 449 aa, synthetic construct
EVQLVESGGGLVQPGRSLTLSCAASGFTFDNFGMHWVRQGPGKGLEWVSGLTWNSGVIGYADSVKGRFTISRDNAKNSLYLQMNSLRPEDTALYYCAKDIRNYGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 563 - protein, 449 aa, synthetic construct
EVQLVESGGGLVQPGGSLRLSCAASGFTFNIFEMNWVRQAPGKGLEWISYISSRGTTTYYADSVRGRFTISRDNAKNSLYLQMNSLRAEDTAVYYCARDYEATIPFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 564 - protein, 451 aa, synthetic construct
EVQLVESGGSVVRPGGSLRLSCEASGFTFDDYGMSWVRQDPGKGLEWVSGINWNGDRTNYADSVKGRFIISRDNAKNSVYLQMNSLRAEDSALYHCARDQGLGVAATLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 565 - protein, 458 aa, synthetic construct
EVQLVESGGGLVQPGGSLRLSCAASGFTVSNYEMNWVRQAPGKGLEWVSYISSSTSNIYYADSVKGRFTISRDNAENSLYLQMNSLRVEDTAVYYCVRDGIVVVPVGRGYYYYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 566 - protein, 456 aa, synthetic construct
QVQLVESGGGVVQPGRSLRLSCAASGFPFSNYVMYWVRQAPGKGLEWVALIFFDGKKNYHADSVKGRFTITRDNSKNMLYLQMNSLRPEDAAVYYCAKIHCPNGVCYKGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 567 - protein, 455 aa, synthetic construct
EVQLVESGGGLVQPGGSLRLSCAASGFTFSNYWMNWVRQAPGKGLEWVANIKEDGGKKLYVDSVKGRFTISRDNAKNSLFLQMNSLRAEDTAVYYCAREDTTLVVDYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 568 - protein, 452 aa, synthetic construct
QVQLVQSGAEVKKPGSSVKVSCKASGGTFNTYAITWVRQAPGQGLEWMGGIIPISGIAEYAQKFQGRVTITTDDSSTTAYMELNSLRSEDTAVYYCASWNYALYYFYGMDVWGRGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 569 - protein, 453 aa, synthetic construct
QVQLVESGGGLVKPGGSLRLSCAASGFTFSVYYMNWIRQAPGKGLEWVSYISSSGSTIYYADSVKGRFTISRDNAKNSLYLQMNSLRAEDTAVYYCGREGYSGTYSYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 570 - protein, 452 aa, synthetic construct
EVQLVESGGGLVQPGRSLRLSCAVSGFTFDDYAMHWVRQAPGKGLEWVSGISWNSGTRGYADSVKGRFTISRDNAKNSLYLQMNSLRGEDTALYYCVKDITISPNYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 571 - protein, 330 aa, synthetic construct [sequence omitted]

(19) United States
(12) Patent Application Publication
(10) Pub. No.: US 2024/0052051 A1
(43) Pub. Date: Feb. 15, 2024

(54) ANTI-TFR:PAYLOAD FUSIONS AND METHODS OF USE THEREOF

(71) Applicant: Regeneron Pharmaceuticals, Inc., Tarrytown, NY (US)

(72) Inventors: Nicole Keating, Yorktown Heights, NY (US); Pascaline Aimé-Wilson, New York, NY (US); John Dugan, Hoboken, NJ (US); Min Gao, Woodcliff Lake, NJ (US); Robert Babb, River Edge, NJ (US); Maria Praggastis, Cortlandt Manor, NY (US); Katherine Cygnar, New York, NY (US); Bojie Zhang, White Plains, NY (US)

(21) Appl. No.: 18/361,367

(22) Filed: Jul. 28, 2023

Related U.S. Application Data
(60) Provisional application No. 63/393,719, filed on Jul. 29, 2022.

(51) Int. Cl. C07K 16/28; A61P 3/00

(52) U.S. Cl. CPC C07K 16/2881; A61P 3/00; C07K 2317/565; C07K 2317/55; C07K 2317/622; C07K 2317/92; C07K 2317/14

(57) ABSTRACT

Provided, in part, are an anti-human transferrin receptor antigen-binding proteins and fusion proteins comprising an anti-human transferrin receptor antigen-binding proteins (e.g., in scFv, Fab or antibody format) which may be fused to a payload for delivery of the payload to a targeted tissue (e.g., past the blood-brain barrier and to the brain). Payloads include, for example, alpha-glucosidase (GAA) polypeptide. Methods for treating various diseases with such molecules, e.g., glycogen storage diseases, such as Pompe Disease, with the fusions are provided.

### CROSS-REFERENCE TO RELATED APPLICATIONS

This application claims the benefit of U.S. Application No. 63/393,719, filed Jul. 29, 2022, which is herein incorporated by reference in its entirety for all purposes.

### REFERENCE TO A SEQUENCE LISTING SUBMITTED AS A TEXT FILE VIA EFS WEB

The Sequence Listing written in file 598926SEQLIST.xml is 573 kilobytes, was created on Jul. 28, 2023, and is hereby incorporated by reference.

### BACKGROUND

Iron delivery to the brain is accomplished via binding and intracellular trafficking of the iron binding protein transferrin (Tf). The Tf receptor (TfR) is a target of some studies to deliver drugs to the brain. For example, approaches include the use of liposomes decorated with Tf used for delivery of imaging agents and DNA (Sharma et al., (2013) Cell penetrating peptide tethered bi-ligand liposomes for delivery to brain in vivo: biodistribution and transfection. J. Control. Release 167, 1-10) or the use of an iron-mimetic peptide as ligand (Staquicini et al., (2011).

A correlation has also been suggested between increased antibody affinity and lysosomal degradation (Bien-Ly et al., (2014) Transferrin receptor (TfR) trafficking determines brain uptake of TfR antibody affinity variants. J. Exp. Med. 211, 233-244) supporting the idea that lower antibody's affinity would help avoid intracellular degradation of the complexes being transported. Bien-Ly et al. found that bispecific antibodies against TfR and beta-secretase (BACE1) traversed the blood-brain barrier (BBB) and effectively reduce brain amyloid beta levels; but also that high-affinity binding to TfR caused a dose-dependent reduction of brain TfR levels. Similarly, Moos & Morgan (2001) compared the ability of anti-TfR antibody, OX26, and transferrin to cross the rat BBB finding that OX26 did not recycle out of the brain as did transferrin because the antibody exhibited a high-affinity antibody-antigen interaction with TfR that is not easily reversed, whereas that of Tf is readily reversed depending on pH and the iron content of Tf (Restricted transport of anti-transferrin receptor antibody (OX26) through the blood-brain barrier in the rat, J Neurochem 2001 October; 79(1):119-29).

### SUMMARY

In one aspect, provided are antigen-binding proteins that bind specifically to transferrin receptor or an antigenic-fragment thereof or variant thereof. Some such antigen-binding proteins comprise: (i) a HCVR that comprises the HCDR1, HCDR2 and HCDR3 of a HCVR comprising the amino acid sequence set forth in SEQ ID NO: 2; 12; 22; 32; 42; 52; 62; 72; 82; 92; 102; 112; 122; 132; 142; 152; 162; 172; 182; 192; 202; 212; 222; 232; 242; 252; 262; 272; 282; 292; 302; or 312 (or a variant thereof); and/or (ii) a LCVR that comprises the LCDR1, LCDR2 and LCDR3 of a LCVR comprising the amino acid sequence set forth in SEQ ID NO: 7; 17; 27; 37; 47; 57; 67; 77; 87; 97; 107; 117; 127; 137; 147; 157; 167; 177; 187; 197; 207; 217; 227; 237; 247; 257; 267; 277; 287; 297; 307; or 317 (or a variant thereof). Optionally, the antigen-binding protein is fused to a payload.

Some such antigen-binding proteins comprise: (1) a HCVR comprising the HCDR1, HCDR2 and HCDR3 of a HCVR that comprises the amino acid sequence set forth in SEQ ID NO: 2 (or a variant thereof); and a LCVR comprising the LCDR1, LCDR2 and LCDR3 of a LCVR that comprises the amino acid sequence set forth in SEQ ID NO: 7 (or a variant thereof); (2) a HCVR comprising the HCDR1, HCDR2 and HCDR3 of a HCVR that comprises the amino acid sequence set forth in SEQ ID NO: 12 (or a variant thereof); and a LCVR comprising the LCDR1, LCDR2 and LCDR3 of a LCVR that comprises the amino acid sequence set forth in SEQ ID NO: 17 (or a variant thereof); (3) a HCVR comprising the HCDR1, HCDR2 and HCDR3 of a HCVR that comprises the amino acid sequence set forth in SEQ ID NO: 22 (or a variant thereof); and a LCVR comprising the LCDR1, LCDR2 and LCDR3 of a LCVR that comprises the amino acid sequence set forth in SEQ ID NO: 27 (or a variant thereof); (4) a HCVR comprising the HCDR1, HCDR2 and HCDR3 of a HCVR that comprises the amino acid sequence set forth in SEQ ID NO: 32 (or a variant thereof); and a LCVR comprising the LCDR1, LCDR2 and LCDR3 of a LCVR that comprises the amino acid sequence set forth in SEQ ID NO: 37 (or a variant thereof); (5) a HCVR comprising the HCDR1, HCDR2 and HCDR3 of a HCVR that comprises the amino acid sequence set forth in SEQ ID NO: 42 (or a variant thereof); and a LCVR comprising the LCDR1, LCDR2 and LCDR3 of a LCVR that comprises the amino acid sequence set forth in SEQ ID NO: 47 (or a variant thereof); (6) a HCVR comprising the HCDR1, HCDR2 and HCDR3 of a HCVR that comprises the amino acid sequence set forth in SEQ ID NO: 52 (or a variant thereof); and a LCVR comprising the LCDR1, LCDR2 and LCDR3 of a LCVR that comprises the amino acid sequence set forth in SEQ ID NO: 57 (or a variant thereof); (7) a HCVR comprising the HCDR1, HCDR2 and HCDR3 of a HCVR that comprises the amino acid sequence set forth in SEQ ID NO: 62 (or a variant thereof); and a LCVR comprising the LCDR1, LCDR2 and LCDR3 of a LCVR that comprises the amino acid sequence set forth in SEQ ID NO: 67 (or a variant thereof); (8) a HCVR comprising the HCDR1, HCDR2 and HCDR3 of a HCVR that comprises the amino acid sequence set forth in SEQ ID NO: 72 (or a variant thereof); and a LCVR comprising the LCDR1, LCDR2 and LCDR3 of a LCVR that comprises the amino acid sequence set forth in SEQ ID NO: 77 (or a variant thereof); (9) a HCVR comprising the HCDR1, HCDR2 and HCDR3 of a HCVR that comprises the amino acid sequence set forth in SEQ ID NO: 82 (or a variant thereof); and a LCVR comprising the LCDR1, LCDR2 and LCDR3 of a LCVR that comprises the amino acid sequence set forth in SEQ ID NO: 87 (or a variant thereof); (10) a HCVR comprising the HCDR1, HCDR2 and HCDR3 of a HCVR that comprises the amino acid sequence set forth in SEQ ID NO: 92 (or a variant thereof); and a LCVR comprising the LCDR1, LCDR2 and LCDR3 of a LCVR that comprises the amino acid sequence set forth in SEQ ID NO: 97 (or a variant thereof); (11) a HCVR comprising the HCDR1, HCDR2 and HCDR3 of a HCVR that comprises the amino acid sequence set forth in SEQ ID NO: 102 (or a variant thereof); and a LCVR comprising the LCDR1, LCDR2 and LCDR3 of a LCVR that comprises the amino acid sequence set forth in SEQ ID NO: 107 (or a variant thereof); (12) a HCVR comprising the HCDR1, HCDR2 and HCDR3 of a HCVR that comprises the amino acid sequence set forth in SEQ ID NO: 112 (or a variant thereof); and a LCVR comprising the LCDR1, LCDR2 and LCDR3 of a LCVR that comprises the amino acid sequence set forth in SEQ ID NO: 117 (or a variant thereof); (13) a HCVR comprising the HCDR1, HCDR2 and HCDR3 of a HCVR that comprises the amino acid sequence set forth in SEQ ID NO: 122 (or a variant thereof); and a LCVR comprising the LCDR1, LCDR2 and LCDR3 of a LCVR that comprises the amino acid sequence set forth in SEQ ID NO: 127 (or a variant thereof); (14) a HCVR comprising the HCDR1, HCDR2 and HCDR3 of a HCVR that comprises the amino acid sequence set forth in SEQ ID NO: 132 (or a variant thereof); and a LCVR comprising the LCDR1, LCDR2 and LCDR3 of a LCVR that comprises the amino acid sequence set forth in SEQ ID NO: 137 (or a variant thereof); (15) a HCVR comprising the HCDR1, HCDR2 and HCDR3 of a HCVR that comprises the amino acid sequence set forth in SEQ ID NO: 142 (or a variant thereof); and a LCVR comprising the LCDR1, LCDR2 and LCDR3 of a LCVR that comprises the amino acid sequence set forth in SEQ ID NO: 147 (or a variant thereof); (16) a HCVR comprising the HCDR1, HCDR2 and HCDR3 of a HCVR that comprises the amino acid sequence set forth in SEQ ID NO: 152 (or a variant thereof); and a LCVR comprising the LCDR1, LCDR2 and LCDR3 of a LCVR that comprises the amino acid sequence set forth in SEQ ID NO: 157 (or a variant thereof); (17) a HCVR comprising the HCDR1, HCDR2 and HCDR3 of a HCVR that comprises the amino acid sequence set forth in SEQ ID NO: 162 (or a variant thereof); and a LCVR comprising the LCDR1, LCDR2 and LCDR3 of a LCVR that comprises the amino acid sequence set forth in SEQ ID NO: 167 (or a variant thereof); (18) a HCVR comprising the HCDR1, HCDR2 and HCDR3 of a HCVR that comprises the amino acid sequence set forth in SEQ ID NO: 172 (or a variant thereof); and a LCVR comprising the LCDR1, LCDR2 and LCDR3 of a LCVR that comprises the amino acid sequence set forth in SEQ ID NO: 177 (or a variant thereof); (19) a HCVR comprising the HCDR1, HCDR2 and HCDR3 of a HCVR that comprises the amino acid sequence set forth in SEQ ID NO: 182 (or a variant thereof); and a LCVR comprising the LCDR1, LCDR2 and LCDR3 of a LCVR that comprises the amino acid sequence set forth in SEQ ID NO: 187 (or a variant thereof); (20) a HCVR comprising the HCDR1, HCDR2 and HCDR3 of a HCVR that comprises the amino acid sequence set forth in SEQ ID NO: 192 (or a variant thereof); and a LCVR comprising the LCDR1, LCDR2 and LCDR3 of a LCVR that comprises the amino acid sequence set forth in SEQ ID NO: 197 (or a variant thereof); (21) a HCVR comprising the HCDR1, HCDR2 and HCDR3 of a HCVR that comprises the amino acid sequence set forth in SEQ ID NO: 202 (or a variant thereof); and a LCVR comprising the LCDR1, LCDR2 and LCDR3 of a LCVR that comprises the amino acid sequence set forth in SEQ ID NO: 207 (or a variant thereof); (22) a HCVR comprising the HCDR1, HCDR2 and HCDR3 of a HCVR that comprises the amino acid sequence set forth in SEQ ID NO: 212 (or a variant thereof); and a LCVR comprising the LCDR1, LCDR2 and LCDR3 of a LCVR that comprises the amino acid sequence set forth in SEQ ID NO: 217 (or a variant thereof); (23) a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof); (24) a HCVR comprising the HCDR1, HCDR2 and HCDR3 of a HCVR that comprises the amino acid sequence set forth in SEQ ID NO: 232 (or a variant thereof); and a LCVR comprising the LCDR1, LCDR2 and LCDR3 of a LCVR that comprises the amino acid sequence set forth in SEQ ID NO: 237 (or a variant thereof); (25) a HCVR comprising the HCDR1, HCDR2 and HCDR3 of a HCVR that comprises the amino acid sequence set forth in SEQ ID NO: 242 (or a variant thereof); and a LCVR comprising the LCDR1, LCDR2 and LCDR3 of a LCVR that comprises the amino acid sequence set forth in SEQ ID NO: 247 (or a variant thereof); (26) a HCVR comprising the HCDR1, HCDR2 and HCDR3 of a HCVR that comprises the amino acid sequence set forth in SEQ ID NO: 252 (or a variant thereof); and a LCVR comprising the LCDR1, LCDR2 and LCDR3 of a LCVR that comprises the amino acid sequence set forth in SEQ ID NO: 257 (or a variant thereof); (27) a HCVR comprising the HCDR1, HCDR2 and HCDR3 of a HCVR that comprises the amino acid sequence set forth in SEQ ID NO: 262 (or a variant thereof); and a LCVR comprising the LCDR1, LCDR2 and LCDR3 of a LCVR that comprises the amino acid sequence set forth in SEQ ID NO: 267 (or a variant thereof); (28) a HCVR comprising the HCDR1, HCDR2 and HCDR3 of a HCVR that comprises the amino acid sequence set forth in SEQ ID NO: 272 (or a variant thereof); and a LCVR comprising the LCDR1, LCDR2 and LCDR3 of a LCVR that comprises the amino acid sequence set forth in SEQ ID NO: 277 (or a variant thereof); (29) a HCVR comprising the HCDR1, HCDR2 and HCDR3 of a HCVR that comprises the amino acid sequence set forth in SEQ ID NO: 282 (or a variant thereof); and a LCVR comprising the LCDR1, LCDR2 and LCDR3 of a LCVR that comprises the amino acid sequence set forth in SEQ ID NO: 287 (or a variant thereof); (30) a HCVR comprising the HCDR1, HCDR2 and HCDR3 of a HCVR that comprises the amino acid sequence set forth in SEQ ID NO: 292 (or a variant thereof); and a LCVR comprising the LCDR1, LCDR2 and LCDR3 of a LCVR that comprises the amino acid sequence set forth in SEQ ID NO: 297 (or a variant thereof); (31) a HCVR comprising the HCDR1, HCDR2 and HCDR3 of a HCVR that comprises the amino acid sequence set forth in SEQ ID NO: 302 (or a variant thereof); and a LCVR comprising the LCDR1, LCDR2 and LCDR3 of a LCVR that comprises the amino acid sequence set forth in SEQ ID NO: 307 (or a variant thereof); and/or (32) a HCVR comprising the HCDR1, HCDR2 and HCDR3 of a HCVR that comprises the amino acid sequence set forth in SEQ ID NO: 312 (or a variant thereof); and a LCVR comprising the LCDR1, LCDR2 and LCDR3 of a LCVR that comprises the amino acid sequence set forth in SEQ ID NO: 317 (or a variant thereof).

Some such antigen-binding proteins comprise a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof). Some such antigen-binding proteins comprise a HCVR comprising the HCDR1, HCDR2 and HCDR3 of a HCVR that comprises the amino acid sequence set forth in SEQ ID NO: 242 (or a variant thereof); and a LCVR comprising the LCDR1, LCDR2 and LCDR3 of a LCVR that comprises the amino acid sequence set forth in SEQ ID NO: 247 (or a variant thereof). Some such antigen-binding proteins comprise a HCVR comprising the HCDR1, HCDR2 and HCDR3 of a HCVR that comprises the amino acid sequence set forth in SEQ ID NO: 132 (or a variant thereof); and a LCVR comprising the LCDR1, LCDR2 and LCDR3 of a LCVR that comprises the amino acid sequence set forth in SEQ ID NO: 137 (or a variant thereof). Some such antigen-binding proteins comprise a HCVR comprising the HCDR1, HCDR2 and HCDR3 of a HCVR that comprises the amino acid sequence set forth in SEQ ID NO: 172 (or a variant thereof); and a LCVR comprising the LCDR1, LCDR2 and LCDR3 of a LCVR that comprises the amino acid sequence set forth in SEQ ID NO: 177 (or a variant thereof). Some such antigen-binding proteins comprise a HCVR comprising the HCDR1, HCDR2 and HCDR3 of a HCVR that comprises the amino acid sequence set forth in SEQ ID NO: 262 (or a variant thereof); and a LCVR comprising the LCDR1, LCDR2 and LCDR3 of a LCVR that comprises the amino acid sequence set forth in SEQ ID NO: 267 (or a variant thereof). Some such antigen-binding proteins comprise a HCVR comprising the HCDR1, HCDR2 and HCDR3 of a HCVR that comprises the amino acid sequence set forth in SEQ ID NO: 272 (or a variant thereof); and a LCVR comprising the LCDR1, LCDR2 and LCDR3 of a LCVR that comprises the amino acid sequence set forth in SEQ ID NO: 277 (or a variant thereof).

Some such antigen-binding proteins comprise a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof).

Some such antigen-binding proteins comprise: (a) a HCVR that comprises: an HCDR1 comprising the amino acid sequence set forth in SEQ ID NO: 3 (or a variant thereof), an HCDR2 comprising the amino acid sequence set forth in SEQ ID NO: 4 (or a variant thereof), and an HCDR3 comprising the amino acid sequence set forth in SEQ ID NO: 5 (or a variant thereof); and a LCVR that comprises: an LCDR1 comprising the amino acid sequence set forth in SEQ ID NO: 8 (or a variant thereof), an LCDR2 comprising the amino acid sequence set forth in SEQ ID NO: 9 (or a variant thereof), and an LCDR3 comprising the amino acid sequence set forth in SEQ ID NO: 10 (or a variant thereof); (b) a HCVR that comprises: an HCDR1 comprising the amino acid sequence set forth in SEQ ID NO: 13 (or a variant thereof), an HCDR2 comprising the amino acid sequence set forth in SEQ ID NO: 14 (or a variant thereof), and an HCDR3 comprising the amino acid sequence set forth in SEQ ID NO: 15 (or a variant thereof); and a LCVR that comprises: an LCDR1 comprising the amino acid sequence set forth in SEQ ID NO: 18 (or a variant thereof), an LCDR2 comprising the amino acid sequence set forth in SEQ ID NO: 19 (or a variant thereof), and an LCDR3 comprising the amino acid sequence set forth in SEQ ID NO: 20 (or a variant thereof); (c) a HCVR that comprises: an HCDR1 comprising the amino acid sequence set forth in SEQ ID NO: 23 (or a variant thereof), an HCDR2 comprising the amino acid sequence set forth in SEQ ID NO: 24 (or a variant thereof), and an HCDR3 comprising the amino acid sequence set forth in SEQ ID NO: 25 (or a variant thereof); and a LCVR that comprises: an LCDR1 comprising the amino acid sequence set forth in SEQ ID NO: 28 (or a variant thereof), an LCDR2 comprising the amino acid sequence set forth in SEQ ID NO: 29 (or a variant thereof), and an LCDR3 comprising the amino acid sequence set forth in SEQ ID NO: 30 (or a variant thereof); (d) a HCVR that comprises: an HCDR1 comprising the amino acid sequence set forth in SEQ ID NO: 33 (or a variant thereof), an HCDR2 comprising the amino acid sequence set forth in SEQ ID NO: 34 (or a variant thereof), and an HCDR3 comprising the amino acid sequence set forth in SEQ ID NO: 35 (or a variant thereof); and a LCVR that comprises: an LCDR1 comprising the amino acid sequence set forth in SEQ ID NO: 38 (or a variant thereof), an LCDR2 comprising the amino acid sequence set forth in SEQ ID NO: 39 (or a variant thereof), and an LCDR3 comprising the amino acid sequence set forth in SEQ ID NO: 40 (or a variant thereof); (e) a HCVR that comprises: an HCDR1 comprising the amino acid sequence set forth in SEQ ID NO: 43 (or a variant thereof), an HCDR2 comprising the amino acid sequence set forth in SEQ ID NO: 44 (or a variant thereof), and an HCDR3 comprising the amino acid sequence set forth in SEQ ID NO: 45 (or a variant thereof); and a LCVR that comprises: an LCDR1 comprising the amino acid sequence set forth in SEQ ID NO: 48 (or a variant thereof), an LCDR2 comprising the amino acid sequence set forth in SEQ ID NO: 49 (or a variant thereof), and an LCDR3 comprising the amino acid sequence set forth in SEQ ID NO: 50 (or a variant thereof); (f) a HCVR that comprises: an HCDR1 comprising the amino acid sequence set forth in SEQ ID NO: 53 (or a variant thereof), an HCDR2 comprising the amino acid sequence set forth in SEQ ID NO: 54 (or a variant thereof), and an HCDR3 comprising the amino acid sequence set forth in SEQ ID NO: 55 (or a variant thereof); and a LCVR that comprises: an LCDR1 comprising the amino acid sequence set forth in SEQ ID NO: 58 (or a variant thereof), an LCDR2 comprising the amino acid sequence set forth in SEQ ID NO: 59 (or a variant thereof), and an LCDR3 comprising the amino acid sequence set forth in SEQ ID NO: 60 (or a variant thereof); (g) a HCVR that comprises: an HCDR1 comprising the amino acid sequence set forth in SEQ ID NO: 63 (or a variant thereof), an HCDR2 comprising the amino acid sequence set forth in SEQ ID NO: 64 (or a variant thereof), and an HCDR3 comprising the amino acid sequence set forth in SEQ ID NO: 65 (or a variant thereof); and a LCVR that comprises: an LCDR1 comprising the amino acid sequence set forth in SEQ ID NO: 68 (or a variant thereof), an LCDR2 comprising the amino acid sequence set forth in SEQ ID NO: 69 (or a variant thereof), and an LCDR3 comprising the amino acid sequence set forth in SEQ ID NO: 70 (or a variant thereof); (h) a HCVR that comprises: an HCDR1 comprising the amino acid sequence set forth in SEQ ID NO: 73 (or a variant thereof), an HCDR2 comprising the amino acid sequence set forth in SEQ ID NO: 74 (or a variant thereof), and an HCDR3 comprising the amino acid sequence set forth in SEQ ID NO: 75 (or a variant thereof); and a LCVR that comprises: an LCDR1 comprising the amino acid sequence set forth in SEQ ID NO: 78 (or a variant thereof), an LCDR2 comprising the amino acid sequence set forth in SEQ ID NO: 79 (or a variant thereof), and an LCDR3 comprising the amino acid sequence set forth in SEQ ID NO: 80 (or a variant thereof); (i) a HCVR that comprises: an HCDR1 comprising the amino acid sequence set forth in SEQ ID NO: 83 (or a variant thereof), an HCDR2 comprising the amino acid sequence set forth in SEQ ID NO: 84 (or a variant thereof), and an HCDR3 comprising the amino acid sequence set forth in SEQ ID NO: 85 (or a variant thereof); and a LCVR that comprises: an LCDR1 comprising the amino acid sequence set forth in SEQ ID NO: 88 (or a variant thereof), an LCDR2 comprising the amino acid sequence set forth in SEQ ID NO: 89 (or a variant thereof), and an LCDR3 comprising the amino acid sequence set forth in SEQ ID NO: 90 (or a variant thereof); (j) a HCVR that comprises: an HCDR1 comprising the amino acid sequence set forth in SEQ ID NO: 93 (or a variant thereof), an HCDR2 comprising the amino acid sequence set forth in SEQ ID NO: 94 (or a variant thereof), and an HCDR3 comprising the amino acid sequence set forth in SEQ ID NO: 95 (or a variant thereof); and a LCVR that comprises: an LCDR1 comprising the amino acid sequence set forth in SEQ ID NO: 98 (or a variant thereof), an LCDR2 comprising the amino acid sequence set forth in SEQ ID NO: 99 (or a variant thereof), and an LCDR3 comprising the amino acid sequence set forth in SEQ ID NO: 100 (or a variant thereof); (k) a HCVR that comprises: an HCDR1 comprising the amino acid sequence set forth in SEQ ID NO: 103 (or a variant thereof), an HCDR2 comprising the amino acid sequence set forth in SEQ ID NO: 104 (or a variant thereof), and an HCDR3 comprising the amino acid sequence set forth in SEQ ID NO: 105 (or a variant thereof); and a LCVR that comprises: an LCDR1 comprising the amino acid sequence set forth in SEQ ID NO: 108 (or a variant thereof), an LCDR2 comprising the amino acid sequence set forth in SEQ ID NO: 109 (or a variant thereof), and an LCDR3 comprising the amino acid sequence set forth in SEQ ID NO: 110 (or a variant thereof); (1) a HCVR that comprises: an HCDR1 comprising the amino acid sequence set forth in SEQ ID NO: 113 (or a variant thereof), an HCDR2 comprising the amino acid sequence set forth in SEQ ID NO: 114 (or a variant thereof), and an HCDR3 comprising the amino acid sequence set forth in SEQ ID NO: 115 (or a variant thereof); and a LCVR that comprises: an LCDR1 comprising the amino acid sequence set forth in SEQ ID NO: 118 (or a variant thereof), an LCDR2 comprising the amino acid sequence set forth in SEQ ID NO: 119 (or a variant thereof), and an LCDR3 comprising the amino acid sequence set forth in SEQ ID NO: 120 (or a variant thereof); (m) a HCVR that comprises: an HCDR1 comprising the amino acid sequence set forth in SEQ ID NO: 123 (or a variant thereof), an HCDR2 comprising the amino acid sequence set forth in SEQ ID NO: 124 (or a variant thereof), and an HCDR3 comprising the amino acid sequence set forth in SEQ ID NO: 125 (or a variant thereof); and a LCVR that comprises: an LCDR1 comprising the amino acid sequence set forth in SEQ ID NO: 128 (or a variant thereof), an LCDR2 comprising the amino acid sequence set forth in SEQ ID NO: 129 (or a variant thereof), and an LCDR3 comprising the amino acid sequence set forth in SEQ ID NO: 130 (or a variant thereof); (n) a HCVR that comprises: an HCDR1 comprising the amino acid sequence set forth in SEQ ID NO: 133 (or a variant thereof), an HCDR2 comprising the amino acid sequence set forth in SEQ ID NO: 134 (or a variant thereof), and an HCDR3 comprising the amino acid sequence set forth in SEQ ID NO: 135 (or a variant thereof); and a LCVR that comprises: an LCDR1 comprising the amino acid sequence set forth in SEQ ID NO: 138 (or a variant thereof), an LCDR2 comprising the amino acid sequence set forth in SEQ ID NO: 139 (or a variant thereof), and an LCDR3 comprising the amino acid sequence set forth in SEQ ID NO: 140 (or a variant thereof); (o) a HCVR that comprises: an HCDR1 comprising the amino acid sequence set forth in SEQ ID NO: 143 (or a variant thereof), an HCDR2 comprising the amino acid sequence set forth in SEQ ID NO: 144 (or a variant thereof), and an HCDR3 comprising the amino acid sequence set forth in SEQ ID NO: 145 (or a variant thereof); and a LCVR that comprises: an LCDR1 comprising the amino acid sequence set forth in SEQ ID NO: 148 (or a variant thereof), an LCDR2 comprising the amino acid sequence set forth in SEQ ID NO: 149 (or a variant thereof), and an LCDR3 comprising the amino acid sequence set forth in SEQ ID NO: 150 (or a variant thereof); (p) a HCVR that comprises: an HCDR1 comprising the amino acid sequence set forth in SEQ ID NO: 153 (or a variant thereof), an HCDR2 comprising the amino acid sequence set forth in SEQ ID NO: 154 (or a variant thereof), and an HCDR3 comprising the amino acid sequence set forth in SEQ ID NO: 155 (or a variant thereof); and a LCVR that comprises: an LCDR1 comprising the amino acid sequence set forth in SEQ ID NO: 158 (or a variant thereof), an LCDR2 comprising the amino acid sequence set forth in SEQ ID NO: 159 (or a variant thereof), and an LCDR3 comprising the amino acid sequence set forth in SEQ ID NO: 160 (or a variant thereof); (q) a HCVR that comprises: an HCDR1 comprising the amino acid sequence set forth in SEQ ID NO: 163 (or a variant thereof), an HCDR2 comprising the amino acid sequence set forth in SEQ ID NO: 164 (or a variant thereof), and an HCDR3 comprising the amino acid sequence set forth in SEQ ID NO: 165 (or a variant thereof); and a LCVR that comprises: an LCDR1 comprising the amino acid sequence set forth in SEQ ID NO: 168 (or a variant thereof), an LCDR2 comprising the amino acid sequence set forth in SEQ ID NO: 169 (or a variant thereof), and an LCDR3 comprising the amino acid sequence set forth in SEQ ID NO: 170 (or a variant thereof); (r) a HCVR that comprises: an HCDR1 comprising the amino acid sequence set forth in SEQ ID NO: 173 (or a variant thereof), an HCDR2 comprising the amino acid sequence set forth in SEQ ID NO: 174 (or a variant thereof), and an HCDR3 comprising the amino acid sequence set forth in SEQ ID NO: 175 (or a variant thereof); and a LCVR that comprises: an LCDR1 comprising the amino acid sequence set forth in SEQ ID NO: 178 (or a variant thereof), an LCDR2 comprising the amino acid sequence set forth in SEQ ID NO: 179 (or a variant thereof), and an LCDR3 comprising the amino acid sequence set forth in SEQ ID NO: 180 (or a variant thereof); (s) a HCVR that comprises: an HCDR1 comprising the amino acid sequence set forth in SEQ ID NO: 183 (or a variant thereof), an HCDR2 comprising the amino acid sequence set forth in SEQ ID NO: 184 (or a variant thereof), and an HCDR3 comprising the amino acid sequence set forth in SEQ ID NO: 185 (or a variant thereof); and a LCVR that comprises: an LCDR1 comprising the amino acid sequence set forth in SEQ ID NO: 188 (or a variant thereof), an LCDR2 comprising the amino acid sequence set forth in SEQ ID NO: 189 (or a variant thereof), and an LCDR3 comprising the amino acid sequence set forth in SEQ ID NO: 190 (or a variant thereof); (t) a HCVR that comprises: an HCDR1 comprising the amino acid sequence set forth in SEQ ID NO: 193 (or a variant thereof), an HCDR2 comprising the amino acid sequence set forth in SEQ ID NO: 194 (or a variant thereof), and an HCDR3 comprising the amino acid sequence set forth in SEQ ID NO: 195 (or a variant thereof); and a LCVR that comprises: an LCDR1 comprising the amino acid sequence set forth in SEQ ID NO: 198 (or a variant thereof), an LCDR2 comprising the amino acid sequence set forth in SEQ ID NO: 199 (or a variant thereof), and an LCDR3 comprising the amino acid sequence set forth in SEQ ID NO: 200 (or a variant thereof); (u) a HCVR that comprises: an HCDR1 comprising the amino acid sequence set forth in SEQ ID NO: 203 (or a variant thereof), an HCDR2 comprising the amino acid sequence set forth in SEQ ID NO: 204 (or a variant thereof), and an HCDR3 comprising the amino acid sequence set forth in SEQ ID NO: 205 (or a variant thereof); and a LCVR that comprises: an LCDR1 comprising the amino acid sequence set forth in SEQ ID NO: 208 (or a variant thereof), an LCDR2 comprising the amino acid sequence set forth in SEQ ID NO: 209 (or a variant thereof), and an LCDR3 comprising the amino acid sequence set forth in SEQ ID NO: 210 (or a variant thereof); (v) a HCVR that comprises: an HCDR1 comprising the amino acid sequence set forth in SEQ ID NO: 213 (or a variant thereof), an HCDR2 comprising the amino acid sequence set forth in SEQ ID NO: 214 (or a variant thereof), and an HCDR3 comprising the amino acid sequence set forth in SEQ ID NO: 215 (or a variant thereof); and a LCVR that comprises: an LCDR1 comprising the amino acid sequence set forth in SEQ ID NO: 218 (or a variant thereof), an LCDR2 comprising the amino acid sequence set forth in SEQ ID NO: 219 (or a variant thereof), and an LCDR3 comprising the amino acid sequence set forth in SEQ ID NO: 220 (or a variant thereof); (w) a HCVR that comprises: an HCDR1 comprising the amino acid sequence set forth in SEQ ID NO: 223 (or a variant thereof), an HCDR2 comprising the amino acid sequence set forth in SEQ ID NO: 224 (or a variant thereof), and an HCDR3 comprising the amino acid sequence set forth in SEQ ID NO: 225 (or a variant thereof); and a LCVR that comprises: an LCDR1 comprising the amino acid sequence set forth in SEQ ID NO: 228 (or a variant thereof), an LCDR2 comprising the amino acid sequence set forth in SEQ ID NO: 229 (or a variant thereof), and an LCDR3 comprising the amino acid sequence set forth in SEQ ID NO: 230 (or a variant thereof); (x) a HCVR that comprises: an HCDR1 comprising the amino acid sequence set forth in SEQ ID NO: 233 (or a variant thereof), an HCDR2 comprising the amino acid sequence set forth in SEQ ID NO: 234 (or a variant thereof), and an HCDR3 comprising the amino acid sequence set forth in SEQ ID NO: 235 (or a variant thereof); and a LCVR that comprises: an LCDR1 comprising the amino acid sequence set forth in SEQ ID NO: 238 (or a variant thereof), an LCDR2 comprising the amino acid sequence set forth in SEQ ID NO: 239 (or a variant thereof), and an LCDR3 comprising the amino acid sequence set forth in SEQ ID NO: 240 (or a variant thereof); (y) a HCVR that comprises: an HCDR1 comprising the amino acid sequence set forth in SEQ ID NO: 243 (or a variant thereof), an HCDR2 comprising the amino acid sequence set forth in SEQ ID NO: 244 (or a variant thereof), and an HCDR3 comprising the amino acid sequence set forth in SEQ ID NO: 245 (or a variant thereof); and a LCVR that comprises: an LCDR1 comprising the amino acid sequence set forth in SEQ ID NO: 248 (or a variant thereof), an LCDR2 comprising the amino acid sequence set forth in SEQ ID NO: 249 (or a variant thereof), and an LCDR3 comprising the amino acid sequence set forth in SEQ ID NO: 250 (or a variant thereof); (z) a HCVR that comprises: an HCDR1 comprising the amino acid sequence set forth in SEQ ID NO: 253 (or a variant thereof), an HCDR2 comprising the amino acid sequence set forth in SEQ ID NO: 254 (or a variant thereof), and an HCDR3 comprising the amino acid sequence set forth in SEQ ID NO: 255 (or a variant thereof); and a LCVR that comprises: an LCDR1 comprising the amino acid sequence set forth in SEQ ID NO: 258 (or a variant thereof), an LCDR2 comprising the amino acid sequence set forth in SEQ ID NO: 259 (or a variant thereof), and an LCDR3 comprising the amino acid sequence set forth in SEQ ID NO: 260 (or a variant thereof); (aa) a HCVR that comprises: an HCDR1 comprising the amino acid sequence set forth in SEQ ID NO: 263 (or a variant thereof), an HCDR2 comprising the amino acid sequence set forth in SEQ ID NO: 264 (or a variant thereof), and an HCDR3 comprising the amino acid sequence set forth in SEQ ID NO: 265 (or a variant thereof); and a LCVR that comprises: an LCDR1 comprising the amino acid sequence set forth in SEQ ID NO: 268 (or a variant thereof), an LCDR2 comprising the amino acid sequence set forth in SEQ ID NO: 269 (or a variant thereof), and an LCDR3 comprising the amino acid sequence set forth in SEQ ID NO: 270 (or a variant thereof); (ab) a HCVR that comprises: an HCDR1 comprising the amino acid sequence set forth in SEQ ID NO: 273 (or a variant thereof), an HCDR2 comprising the amino acid sequence set forth in SEQ ID NO: 274 (or a variant thereof), and an HCDR3 comprising the amino acid sequence set forth in SEQ ID NO: 275 (or a variant thereof); and a LCVR that comprises: an LCDR1 comprising the amino acid sequence set forth in SEQ ID NO: 278 (or a variant thereof), an LCDR2 comprising the amino acid sequence set forth in SEQ ID NO: 279 (or a variant thereof), and an LCDR3 comprising the amino acid sequence set forth in SEQ ID NO: 280 (or a variant thereof); (ac) a HCVR that comprises: an HCDR1 comprising the amino acid sequence set forth in SEQ ID NO: 283 (or a variant thereof), an HCDR2 comprising the amino acid sequence set forth in SEQ ID NO: 284 (or a variant thereof), and an HCDR3 comprising the amino acid sequence set forth in SEQ ID NO: 285 (or a variant thereof); and a LCVR that comprises: an LCDR1 comprising the amino acid sequence set forth in SEQ ID NO: 288 (or a variant thereof), an LCDR2 comprising the amino acid sequence set forth in SEQ ID NO: 289 (or a variant thereof), and an LCDR3 comprising the amino acid sequence set forth in SEQ ID NO: 290 (or a variant thereof); (ad) a HCVR that comprises: an HCDR1 comprising the amino acid sequence set forth in SEQ ID NO: 293 (or a variant thereof), an HCDR2 comprising the amino acid sequence set forth in SEQ ID NO: 294 (or a variant thereof), and an HCDR3 comprising the amino acid sequence set forth in SEQ ID NO: 295 (or a variant thereof); and a LCVR that comprises: an LCDR1 comprising the amino acid sequence set forth in SEQ ID NO: 298 (or a variant thereof), an LCDR2 comprising the amino acid sequence set forth in SEQ ID NO: 299 (or a variant thereof), and an LCDR3 comprising the amino acid sequence set forth in SEQ ID NO: 300 (or a variant thereof); (ae) a HCVR that comprises: an HCDR1 comprising the amino acid sequence set forth in SEQ ID NO: 303 (or a variant thereof), an HCDR2 comprising the amino acid sequence set forth in SEQ ID NO: 304 (or a variant thereof), and an HCDR3 comprising the amino acid sequence set forth in SEQ ID NO: 305 (or a variant thereof); and a LCVR that comprises: an LCDR1 comprising the amino acid sequence set forth in SEQ ID NO: 308 (or a variant thereof), an LCDR2 comprising the amino acid sequence set forth in SEQ ID NO: 309 (or a variant thereof), and an LCDR3 comprising the amino acid sequence set forth in SEQ ID NO: 310 (or a variant thereof); and/or (af) a HCVR that comprises: an HCDR1 comprising the amino acid sequence set forth in SEQ ID NO: 313 (or a variant thereof), an HCDR2 comprising the amino acid sequence set forth in SEQ ID NO: 314 (or a variant thereof), and an HCDR3 comprising the amino acid sequence set forth in SEQ ID NO: 315 (or a variant thereof); and a LCVR that comprises: an LCDR1 comprising the amino acid sequence set forth in SEQ ID NO: 318 (or a variant thereof), an LCDR2 comprising the amino acid sequence set forth in SEQ ID NO: 319 (or a variant thereof), and an LCDR3 comprising the amino acid sequence set forth in SEQ ID NO: 320 (or a variant thereof).

Some such antigen-binding proteins comprise a HCVR that comprises: an HCDR1 comprising the amino acid sequence set forth in SEQ ID NO: 223 (or a variant thereof), an HCDR2 comprising the amino acid sequence set forth in SEQ ID NO: 224 (or a variant thereof), and an HCDR3 comprising the amino acid sequence set forth in SEQ ID NO: 225 (or a variant thereof); and a LCVR that comprises: an LCDR1 comprising the amino acid sequence set forth in SEQ ID NO: 228 (or a variant thereof), an LCDR2 comprising the amino acid sequence set forth in SEQ ID NO: 229 (or a variant thereof), and an LCDR3 comprising the amino acid sequence set forth in SEQ ID NO: 230 (or a variant thereof). Some such antigen-binding proteins comprise a HCVR that comprises: an HCDR1 comprising the amino acid sequence set forth in SEQ ID NO: 243 (or a variant thereof), an HCDR2 comprising the amino acid sequence set forth in SEQ ID NO: 244 (or a variant thereof), and an HCDR3 comprising the amino acid sequence set forth in SEQ ID NO: 245 (or a variant thereof); and a LCVR that comprises: an LCDR1 comprising the amino acid sequence set forth in SEQ ID NO: 248 (or a variant thereof), an LCDR2 comprising the amino acid sequence set forth in SEQ ID NO: 249 (or a variant thereof), and an LCDR3 comprising the amino acid sequence set forth in SEQ ID NO: 250 (or a variant thereof). Some such antigen-binding proteins comprise a HCVR that comprises: an HCDR1 comprising the amino acid sequence set forth in SEQ ID NO: 133 (or a variant thereof), an HCDR2 comprising the amino acid sequence set forth in SEQ ID NO: 134 (or a variant thereof), and an HCDR3 comprising the amino acid sequence set forth in SEQ ID NO: 135 (or a variant thereof); and a LCVR that comprises: an LCDR1 comprising the amino acid sequence set forth in SEQ ID NO: 138 (or a variant thereof), an LCDR2 comprising the amino acid sequence set forth in SEQ ID NO: 139 (or a variant thereof), and an LCDR3 comprising the amino acid sequence set forth in SEQ ID NO: 140 (or a variant thereof). Some such antigen-binding proteins comprise a HCVR that comprises: an HCDR1 comprising the amino acid sequence set forth in SEQ ID NO: 173 (or a variant thereof), an HCDR2 comprising the amino acid sequence set forth in SEQ ID NO: 174 (or a variant thereof), and an HCDR3 comprising the amino acid sequence set forth in SEQ ID NO: 175 (or a variant thereof); and a LCVR that comprises: an LCDR1 comprising the amino acid sequence set forth in SEQ ID NO: 178 (or a variant thereof), an LCDR2 comprising the amino acid sequence set forth in SEQ ID NO: 179 (or a variant thereof), and an LCDR3 comprising the amino acid sequence set forth in SEQ ID NO: 180 (or a variant thereof). Some such antigen-binding proteins comprise a HCVR that comprises: an HCDR1 comprising the amino acid sequence set forth in SEQ ID NO: 263 (or a variant thereof), an HCDR2 comprising the amino acid sequence set forth in SEQ ID NO: 264 (or a variant thereof), and an HCDR3 comprising the amino acid sequence set forth in SEQ ID NO: 265 (or a variant thereof); and a LCVR that comprises: an LCDR1 comprising the amino acid sequence set forth in SEQ ID NO: 268 (or a variant thereof), an LCDR2 comprising the amino acid sequence set forth in SEQ ID NO: 269 (or a variant thereof), and an LCDR3 comprising the amino acid sequence set forth in SEQ ID NO: 270 (or a variant thereof). Some such antigen-binding proteins comprise a HCVR that comprises: an HCDR1 comprising the amino acid sequence set forth in SEQ ID NO: 273 (or a variant thereof), an HCDR2 comprising the amino acid sequence set forth in SEQ ID NO: 274 (or a variant thereof), and an HCDR3 comprising the amino acid sequence set forth in SEQ ID NO: 275 (or a variant thereof); and a LCVR that comprises: an LCDR1 comprising the amino acid sequence set forth in SEQ ID NO: 278 (or a variant thereof), an LCDR2 comprising the amino acid sequence set forth in SEQ ID NO: 279 (or a variant thereof), and an LCDR3 comprising the amino acid sequence set forth in SEQ ID NO: 280 (or a variant thereof).

Some such antigen-binding proteins comprise a HCVR that comprises: an HCDR1 comprising the amino acid sequence set forth in SEQ ID NO: 223 (or a variant thereof), an HCDR2 comprising the amino acid sequence set forth in SEQ ID NO: 224 (or a variant thereof), and an HCDR3 comprising the amino acid sequence set forth in SEQ ID NO: 225 (or a variant thereof); and a LCVR that comprises: an LCDR1 comprising the amino acid sequence set forth in SEQ ID NO: 228 (or a variant thereof), an LCDR2 comprising the amino acid sequence set forth in SEQ ID NO: 229 (or a variant thereof), and an LCDR3 comprising the amino acid sequence set forth in SEQ ID NO: 230 (or a variant thereof).

Some such antigen-binding proteins comprise: (i) a HCVR that comprises the amino acid sequence set forth in SEQ ID NO: 2 (or a variant thereof); and a LCVR that comprises the amino acid sequence set forth in SEQ ID NO: 7 (or a variant thereof); (ii) a HCVR that comprises the amino acid sequence set forth in SEQ ID NO: 12 (or a variant thereof); and a LCVR that comprises the amino acid sequence set forth in SEQ ID NO: 17 (or a variant thereof); (iii) a HCVR that comprises the amino acid sequence set forth in SEQ ID NO: 22 (or a variant thereof); and a LCVR that comprises the amino acid sequence set forth in SEQ ID NO: 27 (or a variant thereof); (iv) a HCVR that comprises the amino acid sequence set forth in SEQ ID NO: 32 (or a variant thereof); and a LCVR that comprises the amino acid sequence set forth in SEQ ID NO: 37 (or a variant thereof); (v) a HCVR that comprises the amino acid sequence set forth in SEQ ID NO: 42 (or a variant thereof); and a LCVR that comprises the amino acid sequence set forth in SEQ ID NO: 47 (or a variant thereof); (vi) a HCVR that comprises the amino acid sequence set forth in SEQ ID NO: 52 (or a variant thereof); and a LCVR that comprises the amino acid sequence set forth in SEQ ID NO: 57 (or a variant thereof); (vii) a HCVR that comprises the amino acid sequence set forth in SEQ ID NO: 62 (or a variant thereof); and a LCVR that comprises the amino acid sequence set forth in SEQ ID NO: 67 (or a variant thereof); (viii) a HCVR that comprises the amino acid sequence set forth in SEQ ID NO: 72 (or a variant thereof); and a LCVR that comprises the amino acid sequence set forth in SEQ ID NO: 77 (or a variant thereof); (ix) a HCVR that comprises the amino acid sequence set forth in SEQ ID NO: 82 (or a variant thereof); and a LCVR that comprises the amino acid sequence set forth in SEQ ID NO: 87 (or a variant thereof); (x) a HCVR that comprises the amino acid sequence set forth in SEQ ID NO: 92 (or a variant thereof); and a LCVR that comprises the amino acid sequence set forth in SEQ ID NO: 97 (or a variant thereof); (xi) a HCVR that comprises the amino acid sequence set forth in SEQ ID NO: 102 (or a variant thereof); and a LCVR that comprises the amino acid sequence set forth in SEQ ID NO: 107 (or a variant thereof); (xii) a HCVR that comprises the amino acid sequence set forth in SEQ ID NO: 112 (or a variant thereof); and a LCVR that comprises the amino acid sequence set forth in SEQ ID NO: 117 (or a variant thereof); (xiii) a HCVR that comprises the amino acid sequence set forth in SEQ ID NO: 122 (or a variant thereof); and a LCVR that comprises the amino acid sequence set forth in SEQ ID NO: 127 (or a variant thereof); (xiv) a HCVR that comprises the amino acid sequence set forth in SEQ ID NO: 132 (or a variant thereof); and a LCVR that comprises the amino acid sequence set forth in SEQ ID NO: 137 (or a variant thereof); (xv) a HCVR that comprises the amino acid sequence set forth in SEQ ID NO: 142 (or a variant thereof); and a LCVR that comprises the amino acid sequence set forth in SEQ ID NO: 147 (or a variant thereof); (xvi) a HCVR that comprises the amino acid sequence set forth in SEQ ID NO: 152 (or a variant thereof); and a LCVR that comprises the amino acid sequence set forth in SEQ ID NO: 157 (or a variant thereof); (xvii) a HCVR that comprises the amino acid sequence set forth in SEQ ID NO: 162 (or a variant thereof); and a LCVR that comprises the amino acid sequence set forth in SEQ ID NO: 167 (or a variant thereof); (xviii) a HCVR that comprises the amino acid sequence set forth in SEQ ID NO: 172 (or a variant thereof); and a LCVR that comprises the amino acid sequence set forth in SEQ ID NO: 177 (or a variant thereof); (xix) a HCVR that comprises the amino acid sequence set forth in SEQ ID NO: 182 (or a variant thereof); and a LCVR that comprises the amino acid sequence set forth in SEQ ID NO: 187 (or a variant thereof); (xx) a HCVR that comprises the amino acid sequence set forth in SEQ ID NO: 192 (or a variant thereof); and a LCVR that comprises the amino acid sequence set forth in SEQ ID NO: 197 (or a variant thereof); (xxi) a HCVR that comprises the amino acid sequence set forth in SEQ ID NO: 202 (or a variant thereof); and a LCVR that comprises the amino acid sequence set forth in SEQ ID NO: 207 (or a variant thereof); (xxii) a HCVR that comprises the amino acid sequence set forth in SEQ ID NO: 212 (or a variant thereof); and a LCVR that comprises the amino acid sequence set forth in SEQ ID NO: 217 (or a variant thereof); (xxiii) a HCVR that comprises the amino acid sequence set forth in SEQ ID NO: 222 (or a variant thereof); and a LCVR that comprises the amino acid sequence set forth in SEQ ID NO: 227 (or a variant thereof); (xxiv) a HCVR that comprises the amino acid sequence set forth in SEQ ID NO: 232 (or a variant thereof); and a LCVR that comprises the amino acid sequence set forth in SEQ ID NO: 237 (or a variant thereof); (xxv) a HCVR that comprises the amino acid sequence set forth in SEQ ID NO: 242 (or a variant thereof); and a LCVR that comprises the amino acid sequence set forth in SEQ ID NO: 247 (or a variant thereof); (xxvi) a HCVR that comprises the amino acid sequence set forth in SEQ ID NO: 252 (or a variant thereof); and a LCVR that comprises the amino acid sequence set forth in SEQ ID NO: 257 (or a variant thereof); (xxvii) a HCVR that comprises the amino acid sequence set forth in SEQ ID NO: 262 (or a variant thereof); and a LCVR that comprises the amino acid sequence set forth in SEQ ID NO: 267 (or a variant thereof); (xxviii) a HCVR that comprises the amino acid sequence set forth in SEQ ID NO: 272 (or a variant thereof); and a LCVR that comprises the amino acid sequence set forth in SEQ ID NO: 277 (or a variant thereof); (xxix) a HCVR that comprises the amino acid sequence set forth in SEQ ID NO: 282 (or a variant thereof); and a LCVR that comprises the amino acid sequence set forth in SEQ ID NO: 287 (or a variant thereof); (xxx) a HCVR that comprises the amino acid sequence set forth in SEQ ID NO: 292 (or a variant thereof); and a LCVR that comprises the amino acid sequence set forth in SEQ ID NO: 297 (or a variant thereof); (xxxi) a HCVR that comprises the amino acid sequence set forth in SEQ ID NO: 302 (or a variant thereof); and a LCVR that comprises the amino acid sequence set forth in SEQ ID NO: 307 (or a variant thereof); and/or (xxxii) a HCVR that comprises the amino acid sequence set forth in SEQ ID NO: 312 (or a variant thereof); and a LCVR that comprises the amino acid sequence set forth in SEQ ID NO: 317 (or a variant thereof).

Some such antigen-binding proteins comprise a HCVR that comprises the amino acid sequence set forth in SEQ ID NO: 222 (or a variant thereof); and a LCVR that comprises the amino acid sequence set forth in SEQ ID NO: 227 (or a variant thereof). Some such antigen-binding proteins comprise a HCVR that comprises the amino acid sequence set forth in SEQ ID NO: 242 (or a variant thereof); and a LCVR that comprises the amino acid sequence set forth in SEQ ID NO: 247 (or a variant thereof). Some such antigen-binding proteins comprise a HCVR that comprises the amino acid sequence set forth in SEQ ID NO: 132 (or a variant thereof); and a LCVR that comprises the amino acid sequence set forth in SEQ ID NO: 137 (or a variant thereof). Some such antigen-binding proteins comprise a HCVR that comprises the amino acid sequence set forth in SEQ ID NO: 172 (or a variant thereof); and a LCVR that comprises the amino acid sequence set forth in SEQ ID NO: 177 (or a variant thereof). Some such antigen-binding proteins comprise a HCVR that comprises the amino acid sequence set forth in SEQ ID NO: 262 (or a variant thereof); and a LCVR that comprises the amino acid sequence set forth in SEQ ID NO: 267 (or a variant thereof). Some such antigen-binding proteins comprise a HCVR that comprises the amino acid sequence set forth in SEQ ID NO: 272 (or a variant thereof); and a LCVR that comprises the amino acid sequence set forth in SEQ ID NO: 277 (or a variant thereof).

Some such antigen-binding proteins comprise a HCVR that comprises the amino acid sequence set forth in SEQ ID NO: 222 (or a variant thereof); and a LCVR that comprises the amino acid sequence set forth in SEQ ID NO: 227 (or a variant thereof).

In some such antigen-binding proteins, the transferrin receptor is the human transferrin receptor or a variant thereof. Some such antigen-binding proteins are an antibody or antigen-binding fragment thereof. Some such antigen-binding proteins are a Fab. Some such antigen-binding proteins are an scFv; optionally wherein the scFv and the payload are connected by a peptide linker which is -(GGGGS)ₘ- (SEQ ID NO: 426); wherein m is 1, 2, 3, 4, 5, 6, 7, 8, 9 or 10; and optionally, wherein the scFv variable regions are connected by a peptide linker which is -(GGGGS)ₙ- (SEQ ID NO: 426); wherein n is 1, 2, 3, 4, 5, 6, 7, 8, 9 or 10. In some such antigen-binding proteins, the scFv comprises the amino acid sequence set forth in SEQ ID NO: 443 (or a variant thereof), comprises the amino acid sequence set forth in SEQ ID NO: 440 (or a variant thereof), comprises the amino acid sequence set forth in SEQ ID NO: 429 (or a variant thereof), comprises the amino acid sequence set forth in SEQ ID NO: 433 (or a variant thereof), comprises the amino acid sequence set forth in SEQ ID NO: 442 (or a variant thereof), or comprises the amino acid sequence set forth in SEQ ID NO: 438 (or a variant thereof). In some such antigen-binding proteins, the scFv comprises the amino acid sequence set forth in SEQ ID NO: 443 (or a variant thereof).

Also provided are antigen-binding proteins that bind specifically to transferrin receptor or an antigenic-fragment thereof or variant thereof and bind to one or more epitopes of hTfR selected from: (a) an epitope comprising the sequence LLNE (SEQ ID NO: 525) and/or an epitope comprising the sequence TYKEL (SEQ ID NO: 507); (b) an epitope comprising the sequence DSTDFTGT (SEQ ID NO: 526) and/or an epitope comprising the sequence VKHPVTGQF (SEQ ID NO: 527) and/or an epitope comprising the sequence IERIPEL (SEQ ID NO: 528); (c) an epitope comprising the sequence LNENSYVPREAGSQKDEN (SEQ ID NO: 529); (d) an epitope comprising the sequence FEDL (SEQ ID NO: 519); (e) an epitope comprising the sequence IVDKNGRL (SEQ ID NO: 530); (f) an epitope comprising the sequence IVDKNGRLVY (SEQ ID NO: 531); (g) an epitope comprising the sequence DQTKF (SEQ ID NO: 532); (h) an epitope comprising the sequence LVENPGGY (SEQ ID NO: 533) and/or an epitope comprising the sequence PIVNAELSF (SEQ ID NO: 534) and/or an epitope comprising the sequence PYLGTTMDT (SEQ ID NO: 535); (i) an epitope comprising the sequence LLNENSYVPREAGSQKDENLAL (SEQ ID NO: 505) and/or an epitope comprising the sequence IYMDQTKFPIVNAEL (SEQ ID NO: 506) and/or an epitope comprising the sequence TYKEL (SEQ ID NO: 507); (j) an epitope comprising the sequence KRKLSEKLDSTDFTGTIKL (SEQ ID NO: 508) and/or an epitope comprising the sequence YTLIEKTMQNVKHPVTGQFL (SEQ ID NO: 509) and/or an epitope comprising the sequence LIERIPELNKVARAAAE (SEQ ID NO: 510); (k) an epitope comprising the sequence LNENSYVPREAGSQKDENL (SEQ ID NO: 511); (1) an epitope comprising the sequence GTKKDFEDL (SEQ ID NO: 512); (m) an epitope comprising the sequence SVIIVDKNGRLVYLVENPGGYVAYSK (SEQ ID NO: 513); (n) an epitope comprising the sequence LLNENSYVPREAGSQKDEN (SEQ ID NO: 514) and/or an epitope comprising the sequence DQTKFPIVNAEL (SEQ ID NO: 515) and/or an epitope comprising the sequence TYKELIERIPELNK (SEQ ID NO: 516); (o) an epitope comprising the sequence LLNENSYVPREAGSQKDEN (SEQ ID NO: 514) and/or an epitope comprising the sequence TYKELIERIPELNK (SEQ ID NO: 516); (p) an epitope comprising the sequence SVIIVDKNGRLVYLVENPGGYVAY (SEQ ID NO: 517); (q) an epitope comprising the sequence IYMDQTKFPIVNAEL (SEQ ID NO: 506) and/or an epitope comprising the sequence FGNMEGDCPSDWKTDSTCRM (SEQ ID NO: 518); (r) an epitope comprising the sequence LLNENSYVPREAGSQKDENLAL (SEQ ID NO: 505) and/or an epitope comprising the sequence LVENPGGYVAYSKAATVTGKL (SEQ ID NO: 520) and/or an epitope comprising the sequence IYMDQTKFPIVNAELSF (SEQ ID NO: 521) and/or an epitope comprising the sequence ISRAAAEKL (SEQ ID NO: 522) and/or an epitope comprising the sequence VTSESKNVKLTVSNVLKE (SEQ ID NO: 523) and/or an epitope comprising the sequence FCEDTDYPYLGTTMDT (SEQ ID NO: 524); (s) an epitope comprised within or overlapping with the sequence LLNENSYVPREAGSQKDENLAL (SEQ ID NO: 505) and/or an epitope comprised within or overlapping with the sequence IYMDQTKFPIVNAEL (SEQ ID NO: 506) and/or an epitope comprised within or overlapping with the sequence TYKEL (SEQ ID NO: 507); (t) an epitope comprised within or overlapping with the sequence KRKLSEKLDSTDFTGTIKL (SEQ ID NO: 508) and/or an epitope comprised within or overlapping with the sequence YTLIEKTMQNVKHPVTGQFL (SEQ ID NO: 509) and/or an epitope comprised within or overlapping with the sequence LIERIPELNKVARAAAE (SEQ ID NO: 510); (u) an epitope comprised within or overlapping with the sequence LNENSYVPREAGSQKDENL (SEQ ID NO: 511); (v) an epitope comprised within or overlapping with the sequence GTKKDFEDL (SEQ ID NO: 512); (w) an epitope comprised within or overlapping with the sequence SVIIVDKNGRLVYLVENPGGYVAYSK (SEQ ID NO: 513); (x) an epitope comprised within or overlapping with the sequence LLNENSYVPREAGSQKDEN (SEQ ID NO: 514) and/or an epitope comprised within or overlapping with the sequence DQTKFPIVNAEL (SEQ ID NO: 515) and/or an epitope comprised within or overlapping with the sequence TYKELIERIPELNK (SEQ ID NO: 516); (y) an epitope comprised within or overlapping with the sequence LLNENSYVPREAGSQKDEN (SEQ ID NO: 514) and/or an epitope comprised within or overlapping with the sequence TYKELIERIPELNK (SEQ ID NO: 516); (z) an epitope comprised within or overlapping with the sequence SVIIVDKNGRLVYLVENPGGYVAY (SEQ ID NO: 517); (aa) an epitope comprised within or overlapping with the sequence IYMDQTKFPIVNAEL (SEQ ID NO: 506) and/or an epitope comprised within or overlapping with the sequence FGNMEGDCPSDWKTDSTCRM (SEQ ID NO: 518); and (ab) an epitope comprised within or overlapping with the sequence LLNENSYVPREAGSQKDENLAL (SEQ ID NO: 505) and/or an epitope comprised within or overlapping with the sequence LVENPGGYVAYSKAATVTGKL (SEQ ID NO: 520) and/or an epitope comprised within or overlapping with the sequence IYMDQTKFPIVNAELSF (SEQ ID NO: 521) and/or an epitope comprised within or overlapping with the sequence ISRAAAEKL (SEQ ID NO: 522) and/or an epitope comprised within or overlapping with the sequence VTSESKNVKLTVSNVLKE (SEQ ID NO: 523) and/or an epitope comprised within or overlapping with the sequence FCEDTDYPYLGTTMDT (SEQ ID NO: 524). In some such antigen-binding proteins, the antigen binding protein comprises an antibody or antigen-binding fragment thereof which binds to one or more epitopes of hTfR selected from: (a) an epitope consisting of the sequence LLNE (SEQ ID NO: 525) and/or an epitope consisting of the sequence TYKEL (SEQ ID NO: 507); (b) an epitope consisting of the sequence DSTDFTGT (SEQ ID NO: 526) and/or an epitope consisting of the sequence VKHPVTGQF (SEQ ID NO: 527) and/or an epitope consisting of the sequence IERIPEL (SEQ ID NO: 528); (c) an epitope consisting of the sequence LNENSYVPREAGSQKDEN (SEQ ID NO: 529); (d) an epitope consisting of the sequence FEDL (SEQ ID NO: 519); (e) an epitope consisting of the sequence IVDKNGRL (SEQ ID NO: 530); (f) an epitope consisting of the sequence IVDKNGRLVY (SEQ ID NO: 531); (g) an epitope consisting of the sequence DQTKF (SEQ ID NO: 532); (h) an epitope consisting of the sequence LVENPGGY (SEQ ID NO: 533) and/or an epitope consisting of the sequence PIVNAELSF (SEQ ID NO: 534) and/or an epitope consisting of the sequence PYLGTTMDT (SEQ ID NO: 535); (i) an epitope consisting of the sequence LLNENSYVPREAGSQKDENLAL (SEQ ID NO: 505) and/or an epitope consisting of the sequence IYMDQTKFPIVNAEL (SEQ ID NO: 506) and/or an epitope consisting of the sequence TYKEL (SEQ ID NO: 507); (j) an epitope consisting of the sequence KRKLSEKLDSTDFTGTIKL (SEQ ID NO: 508) and/or an epitope consisting of the sequence YTLIEKTMQNVKHPVTGQFL (SEQ ID NO: 509) and/or an epitope consisting of the sequence LIERIPELNKVARAAAE (SEQ ID NO: 510); (k) an epitope consisting of the sequence LNENSYVPREAGSQKDENL (SEQ ID NO: 511); (1) an epitope consisting of the sequence GTKKDFEDL (SEQ ID NO: 512); (m) an epitope consisting of the sequence SVIIVDKNGRLVYLVENPGGYVAYSK (SEQ ID NO: 513); (n) an epitope consisting of the sequence LLNENSYVPREAGSQKDEN (SEQ ID NO: 514) and/or an epitope consisting of the sequence DQTKFPIVNAEL (SEQ ID NO: 515) and/or an epitope consisting of the sequence TYKELIERIPELNK (SEQ ID NO: 516); (o) an epitope consisting of the sequence LLNENSYVPREAGSQKDEN (SEQ ID NO: 514) and/or an epitope consisting of the sequence TYKELIERIPELNK (SEQ ID NO: 516); (p) an epitope consisting of the sequence SVIIVDKNGRLVYLVENPGGYVAY (SEQ ID NO: 517); (q) an epitope consisting of the sequence IYMDQTKFPIVNAEL (SEQ ID NO: 506) and/or an epitope consisting of the sequence FGNMEGDCPSDWKTDSTCRM (SEQ ID NO: 518); and (r) an epitope consisting of the sequence LLNENSYVPREAGSQKDENLAL (SEQ ID NO: 505) and/or an epitope consisting of the sequence LVENPGGYVAYSKAATVTGKL (SEQ ID NO: 520) and/or an epitope consisting of the sequence IYMDQTKFPIVNAELSF (SEQ ID NO: 521) and/or an epitope consisting of the sequence ISRAAAEKL (SEQ ID NO: 522) and/or an epitope consisting of the sequence VTSESKNVKLTVSNVLKE (SEQ ID NO: 523) and/or an epitope consisting of the sequence FCEDTDYPYLGTTMDT (SEQ ID NO: 524).

In some such antigen-binding proteins, the antigen-binding protein is selected from a humanized antibody or antigen binding fragment thereof, human antibody or antigen binding fragment thereof, murine antibody or antigen binding fragment thereof, chimeric antibody or antigen binding fragment thereof, monovalent Fab′, divalent Fab2, F(ab)′3 fragments, single-chain fragment variable (scFv), bis-scFv, (scFv)2, diabody, bivalent antibody, one-armed antibody, minibody, nanobody, triabody, tetrabody, disulfide stabilized Fv protein (dsFv), single-domain antibody (sdAb), Ig NAR, single heavy chain antibody, bispecific antibody or biding fragment thereof, bi-specific T-cell engager (BiTE), trispecific antibody, or chemically modified derivatives thereof.

In another aspect, provided is a fusion protein comprising any of the above antigen-binding proteins fused to a payload. In another aspect, provided is a fusion protein comprising an antigen-binding protein that binds specifically to transferrin receptor or an antigenic-fragment thereof or variant thereof fused to a payload, wherein the antigen-binding protein binds to human transferrin receptor with a K_{D} of about 41 nM or a stronger affinity. In some such fusion proteins, the antigen-binding protein binds to human transferrin receptor with a K_{D} of about 41 nM or a stronger affinity. In some such fusion proteins, the antigen-binding protein binds to human transferrin receptor with a K_{D} of about 3 nM or a stronger affinity. In some such fusion proteins, the antigen-binding protein binds to human transferrin receptor with a K_{D} of about 3 nM or a stronger affinity, or wherein the antigen-binding protein binds to human transferrin receptor with a K_{D} of about 0.45 nM to 3 nM. In some such fusion proteins, the payload is one or more antibodies or antigen-binding fragments thereof, proteins, enzymes or viral vectors containing one or more polynucleotides or oligonucleotides; or human alpha-glucosidase polypeptide (hGAA) or a variant thereof. In some such fusion proteins, the payload is a lysosomal storage disease therapeutic agent (LSD-TA); or a polypeptide or a polypeptide encoded by a human gene specified in any one of Tables C-N or a variant thereof. In some such fusion proteins, the payload is an LSD-TA which is Miglustat, Eliglustat, α-galactosidase A; ceramidase; β-glucosidase; saposin-C activator; acid sphingomyelinase; β-galactosidase; β-hexosaminidase A and B; β-hexosaminidase A; GM2-activator protein; GM3 synthase; arylsulfatase A; sphingolipid activator; α-iduronidase; iduronidase-2-sulphatase; heparan N-sulphatase; N-acetyl-α-glucosaminidase; acetyl-CoA; α-glucosamide N-acetyltransferase; N-acetylglucosamine-6-sulphatase; N-acetylgalactosamine-6-sulphate sulphatase; β-galactosidase; N-acetylgalactosamine-4-sulphatase (arylsulphatase B); β-glucuronidase; hylauronidase; α-hlucosidase 2; or lysosomal acid lipase.

Some such fusion proteins are a fusion protein comprising an antigen-binding protein that binds specifically to human transferrin receptor, which comprises a heavy chain variable region (HCVR or V_{H}) and a light chain variable region (LCVR or V_{L}), which is fused to an alpha-glucosidase polypeptide (GAA), wherein a Fab having said V_{H} and V_{L} binds to human transferrin receptor with a K_{D} of about 0.65 nM or a greater affinity; and wherein, when said fusion protein is administered to a mouse expressing human transferrin receptor in the brain, the mouse achieves a molar ratio of mature GAA protein in the brain:serum GAA protein, in the mouse, of about 1:1 or greater when normalized against said ratio in mouse expressing mouse transferrin receptor that was administered 8D3.

In some such fusion proteins, the antigen-binding protein is a Fab. In some such fusion proteins, the antigen-binding protein is a single chain fragment variable (scFv). In some such fusion proteins, the fusion protein comprises the amino acid sequence set forth in SEQ ID NO: 408 (or a variant thereof) or comprises the amino acid sequence set forth in SEQ ID NO: 405 (or a variant thereof). In some such fusion proteins, the fusion protein comprises the amino acid sequence set forth in SEQ ID NO: 408 (or a variant thereof).

In some such fusion proteins, the antigen-binding protein is an antibody or antigen-binding fragment thereof. In some such fusion proteins, the antigen-binding protein is an scFv comprising domains arranged in the following orientation: N-Heavy chain variable region-Light chain variable region-GAA protein-C. In some such fusion proteins, the antigen-binding protein is an scFv comprising domains arranged in the following orientation: N-Light chain variable region-Heavy chain variable region-GAA protein-C. In some such fusion proteins, the antigen-binding protein is an scFv, wherein said scFv and GAA are connected by a peptide linker. In some such fusion proteins, the scFv and GAA are connected by a peptide linker which is -(GGGGS)ₘ- (SEQ ID NO: 426); wherein m is 1, 2, 3, 4, 5, 6, 7, 8, 9 or 10. In some such fusion proteins, the antigen-binding protein is an scFv and said scFv variable regions are connected by a peptide linker. In some such fusion proteins, the scFv variable regions are connected by a peptide linker which is -(GGGGS)ₙ- (SEQ ID NO: 426); wherein n is 1, 2, 3, 4, 5, 6, 7, 8, 9 or 10. In some such fusion proteins, the fusion protein binds to human transferrin receptor with a K_{D} of about 1×10⁻⁷ M or a greater affinity.

Some such fusion proteins comprise: (i) a HCVR that comprises the HCDR1, HCDR2 and HCDR3 of a HCVR comprising the amino acid sequence set forth in SEQ ID NO: 2; 462; 12; 463; 22; 464; 32; 42; 52; 467; 62; 492; 72; 470; 82; 92; 472; 102; 112; 473; 122; 132; 142; 475; 152; 162; 477; 172; 182; 478; 192; 480; 202; 481; 212; 222; 232; 242; 252; 482; 262; 272; 282; 292; 302; 483 or 312 (or a variant thereof); and/or (ii) a LCVR that comprises the LCDR1, LCDR2 and LCDR3 of a LCVR comprising the amino acid sequence set forth in SEQ ID NO: 7; 17; 27; 37; 465; 47; 466; 57; 468; 67; 469; 77; 471; 87; 97; 107; 117; 474; 127; 137; 147; 476; 157; 167; 177; 187; 479; 197; 207; 217; 227; 237; 247; 257; 267; 277; 287; 297; 307; 488; 317 or 484 (or a variant thereof).

In some such fusion proteins, the fusion protein comprises an scFv that comprises a heavy chain variable region (V_{H}) and a light chain variable region (V_{L}), and an alpha-glucosidase polypeptide (GAA), wherein said V_{H}, V_{L} and GAA are arranged as follows: (i) V_{L}-V_{H}-GAA; (ii) V_{H}-V_{L}-GAA; (iii) V_{L}-[(GGGGS)₃ (SEQ ID NO: 538)]-V_{H}-[(GGGGS)₂ (SEQ ID NO: 537)]-GAA; or (iv) V_{H}-[(GGGGS)₃ (SEQ ID NO: 538)]-V_{L}-[(GGGGS)₂ (SEQ ID NO: 537)]-GAA.

Some such fusion proteins comprise the amino acid sequence set forth in a member selected from the group consisting of SEQ ID NOs: 392-423; SEQ ID NO: 321 (optionally lacking the N-terminal MHRPRRRGTRPPPLALLAALLLAARGADA (SEQ ID NO: 500) signal sequence); SEQ ID NO: 322 (optionally lacking the N-terminal MHRPRRRGTRPPPLALLAALLLAARGADA (SEQ ID NO: 500) signal sequence); SEQ ID NO: 323 (optionally lacking the N-terminal MHRPRRRGTRPPPLALLAALLLAARGADA (SEQ ID NO: 500) signal sequence); and SEQ ID NO: 324 (optionally lacking the N-terminal MHRPRRRGTRPPPLALLAALLLAARGADA (SEQ ID NO: 500) signal sequence); or a variant thereof.

In some such fusion proteins, the antigen-binding protein, which when not fused to a GAA polypeptide, does not block more than 50% of binding of a human transferrin receptor C-terminal fragment to human holo-transferrin that occurs in the absence of such single chain fragment variable (scFv), antibody or an antigen-binding fragment. In some such fusion proteins, the blocking is as measured in an Enzyme Linked Immunosorbent Assay (ELISA) plate assay wherein binding of human transferrin receptor extracellular domain that is fused to a His6-myc-myc tag is pre-bound to said scFv, antibody or antigen-binding fragment and then contacted with holo-transferrin which is immobilized to the surface of the plate by binding of an anti-holo-transferrin antibody that is bound to the plate. In some such fusion proteins, binding of the holo-transferrin and human transferrin receptor extracellular domain in the absence of the scFv, antibody or antigen-binding fragment is measured at a concentration of about 300 pM human transferrin receptor extracellular domain.

Some such fusion proteins or antigen-binding proteins bind specifically to human transferrin receptor which has one or more of the following characteristics:
affinity (K_{D}) for binding to human TfR at 25° C. in surface plasmon resonance format of about 41 nM or a higher affinity;
affinity (K_{D}) for binding to monkey TfR at 25° C. in surface plasmon resonance format of about 0 nM (no detectable binding) or a higher affinity;
ratio of [K_{D} for binding to monkey TfR/K_{D} for binding to human TfR] at 25° C. in surface plasmon resonance format of from 0 to 278;
blocks about 3-13% hTfR binding to Human Holo-Tf when in Fab format (IgG1);
blocks about 6-13% hTfR binding to Human Holo-Tf when in scFv (V_{K}—V_{H}) format;
blocks about 11-26% hTfR binding to Human Holo-Tf when in scFv (V_{H}-V_{L}) format;
when in anti-hTfR scFv:hGAA format, exhibits a ratio of about 1-2 mature hGAA protein in brain (normalized to that of positive control 8D3:GAA scFv) when administered to mice expressing human transferrin receptor (optionally, lacking functional endogenous GAA);
when in anti-hTfR scFv:hGAA format, exhibits a ratio of about 0.1-1.2 mature hGAA protein in brain parenchyma (normalized to that of positive control 8D3:GAA scFv) when administered to mice expressing human transferrin receptor (optionally, lacking functional endogenous GAA);
when in anti-hTfR scFv:hGAA format, exhibits a ratio of about 0.67, 1.80, 1.78 or 7.74 (about 1-2) mature hGAA protein in quadriceps (normalized to that of positive control 8D3:GAA scFv) when administered to mice expressing human transferrin receptor (optionally, lacking functional endogenous GAA);
when in anti-hTfR scFv:hGAA format, exhibits a ratio of about 0.1-1.2 mature hGAA protein in brain parenchyma (normalized to that of positive control 8D3:GAA scFv) when administered to mice expressing human transferrin receptor (optionally, lacking functional endogenous GAA);
when in anti-hTfR scFv:hGAA format, delivers mature hGAA protein to serum, liver, cerebrum, cerebellum, spinal cord, heart and/or quadricep when administered to mice expressing human transferrin receptor (optionally, lacking functional endogenous GAA);
when in anti-hTfR scFv:hGAA format, reduces glycogen stored in cerebrum, cerebellum, spinal cord, heart and/or quadricep when administered to mice expressing human transferrin receptor (optionally, lacking functional endogenous GAA);
when comprising the antigen-binding protein fused to GAA, reduces glycogen levels in cerebellum of mice expressing human transferrin receptor but lacking functional endogenous GAA by at least about 90% relative to that of untreated mice;
when comprising the antigen-binding protein fused to GAA, reduces glycogen levels in quadricep of mice expressing human transferrin receptor but lacking functional endogenous GAA by at least about 89% relative to that of untreated mice; and/or
does not cause abnormal iron homeostasis when administered to mice expressing human transferrin receptor.

In another aspect, provided are pharmaceutical compositions comprising any of the above fusion proteins or antigen-binding proteins and a pharmaceutically acceptable carrier.

In another aspect, provided are compositions or kits comprising any of the above fusion proteins or antigen-binding proteins or pharmaceutical compositions in association with a further therapeutic agent. In some such compositions or kits, the further therapeutic agent is selected from: alglucosidase alfa, rituximab, methotrexate, intravenous immunoglobulin (IVIG), avalglucosidase alfa, levalbuterol, an antibiotic, cortisone, prednisone, a bisphosphonate, and palivizumab. In some such compositions or kits, the further therapeutic agent is selected from: a beta2-adrenergic agonist, a steroid, a bisphosphonate, an infectious disease treatment, a vaccine, and a Pneumococcal vaccine.

In another aspect, provided is a complex comprising any of the above fusion proteins or antigen-binding proteins bound to a human transferrin receptor polypeptide or antigenic fragment thereof.

In another aspect, provided are isolated polynucleotide encoding any of the above fusion proteins or antigen-binding proteins. Some such polynucleotides comprise the nucleotide sequence set forth in SEQ ID NO: 1; 6; 11; 16; 21; 26; 31; 36; 41; 46; 51; 56; 61; 66; 71; 76; 81; 86; 91; 96; 101; 106; 111; 116; 121; 126; 131; 136; 141; 146; 151; 156; 161; 166; 171; 176; 181; 186; 191; 196; 201; 206; 211; 216; 221; 226; 231; 236; 241; 246; 251; 256; 261; 266; 271; 276; 281; 286; 291; 296; 301; 306; 311; and/or 316. Some such polynucleotides comprise: (1) the nucleotide sequence set forth in SEQ ID NO: 1 and SEQ ID NO: 6; (2) the nucleotide sequence set forth in SEQ ID NO: 11 and SEQ ID NO: 16; (3) the nucleotide sequence set forth in SEQ ID NO: 21 and SEQ ID NO: 26; (4) the nucleotide sequence set forth in SEQ ID NO: 31 and SEQ ID NO: 36; (5) the nucleotide sequence set forth in SEQ ID NO: 41 and SEQ ID NO: 46; (6) the nucleotide sequence set forth in SEQ ID NO: 51 and SEQ ID NO: 56; (7) the nucleotide sequence set forth in SEQ ID NO: 61 and SEQ ID NO: 66; (8) the nucleotide sequence set forth in SEQ ID NO: 71 and SEQ ID NO: 76; (9) the nucleotide sequence set forth in SEQ ID NO: 81 and SEQ ID NO: 86; (10) the nucleotide sequence set forth in SEQ ID NO: 91 and SEQ ID NO: 96; (11) the nucleotide sequence set forth in SEQ ID NO: 101 and SEQ ID NO: 106; (12) the nucleotide sequence set forth in SEQ ID NO: 111 and SEQ ID NO: 116; (13) the nucleotide sequence set forth in SEQ ID NO: 121 and SEQ ID NO: 126; (14) the nucleotide sequence set forth in SEQ ID NO: 131 and SEQ ID NO: 136; (15) the nucleotide sequence set forth in SEQ ID NO: 141 and SEQ ID NO: 146; (16) the nucleotide sequence set forth in SEQ ID NO: 151 and SEQ ID NO: 156; (17) the nucleotide sequence set forth in SEQ ID NO: 161 and SEQ ID NO: 166; (18) the nucleotide sequence set forth in SEQ ID NO: 171 and SEQ ID NO: 176; (19) the nucleotide sequence set forth in SEQ ID NO: 181 and SEQ ID NO: 186; (20) the nucleotide sequence set forth in SEQ ID NO: 191 and SEQ ID NO: 196; (21) the nucleotide sequence set forth in SEQ ID NO: 201 and SEQ ID NO: 206; (22) the nucleotide sequence set forth in SEQ ID NO: 211 and SEQ ID NO: 216; (23) the nucleotide sequence set forth in SEQ ID NO: 221 and SEQ ID NO: 226; (24) the nucleotide sequence set forth in SEQ ID NO: 231 and SEQ ID NO: 236; (25) the nucleotide sequence set forth in SEQ ID NO: 241 and SEQ ID NO: 246; (26) the nucleotide sequence set forth in SEQ ID NO: 251 and SEQ ID NO: 256; (27) the nucleotide sequence set forth in SEQ ID NO: 261 and SEQ ID NO: 266; (28) the nucleotide sequence set forth in SEQ ID NO: 271 and SEQ ID NO: 276; (29) the nucleotide sequence set forth in SEQ ID NO: 281 and SEQ ID NO: 286; (30) the nucleotide sequence set forth in SEQ ID NO: 291 and SEQ ID NO: 296; (31) the nucleotide sequence set forth in SEQ ID NO: 301 and SEQ ID NO: 306; and/or (32) the nucleotide sequence set forth in SEQ ID NO: 311 and SEQ ID NO: 316.

In another aspect, provided are vectors comprising any of the above polynucleotides.

In another aspect, provided are host cells comprising any of the above fusion proteins, antigen-binding proteins, polynucleotides, or vectors. Some such host cells are a Chinese hamster ovary (CHO) cell.

In another aspect, provided are methods for making any of the above fusion proteins or antigen-binding proteins, comprising culturing a host cell comprising a polynucleotide that encodes the fusion protein or antigen-binding protein in a culture medium under conditions favorable to expression of the fusion protein or antigen-binding protein. Some such methods comprise the steps: (a) introducing said polynucleotide into a host cell; (b) culturing the host cell under conditions favorable to expression of the fusion protein or antigen-binding protein; and (c) optionally, isolating the fusion protein or antigen-binding protein from the culture medium and/or host cell; and (d) optionally, chemically conjugating the antigen-binding protein to a payload. In another aspect, provided are fusion proteins or antigen-binding proteins which are the product of such methods.

In another aspect, provided are vessels or injection devices comprising any of the above fusion proteins or antigen-binding proteins.

In another aspect, provided are methods for administering any of the above fusion proteins or antigen-binding proteins to a subject comprising introducing the protein into the body of the subject. In some such methods, the fusion protein or antigen-binding protein is introduced into the body of the subject parenterally.

In another aspect, provided are methods for treating or preventing a lysosomal storage disease in a subject in need thereof comprising administering, to the subject, an effective amount of any of the above fusion proteins, wherein the payload is a lysosomal storage disease therapeutic agent (LSD-TA). In some such methods, the lysosomal storage disease is: Fabry disease; Farber lipogranulomatosis; Gaucher disease type I; Gaucher disease (type II or III); Niemann-Pick diseases (type A or B); GM1-gangliosidosis; GM2-gangliosidosis (Sandhoff); GM2-gangliosidosis (Tay-Sachs); GM2-gangliosidosis (GM2-activator deficiency); GM3-gangliosidosis; Metachromatic leukodystrophy; Sphingolipid-activator deficiency; MPS I (Scheie, Hurler-Scheie, or Hurler disease); MPS II (Hunter); MPS IIIA (Sanfilippo A); MPS IIIB (Sanfilippo B); MPS IIIC (Sanfilippo C); MPS IIID (Sanfilippo D); MPS IVA (Morquio syndrome A); MPS IVB (Morquio syndrome B); MPS VI (Maroteaux-Lamy); MPS VII (Sly disease); MPS IX; Pompe (glycogen storage disease type II); or Lysosomal acid lipase deficiency (LAL-D; Wolman disease). In some such methods, one or more signs or symptoms of the LSD in the subject are alleviated after the fusion protein or antigen-binding protein is administered.

In another aspect, provided are methods for treating or preventing a glycogen storage disease (GSD)) in a subject in need thereof comprising administering, to the subject, an effective amount of any of the above fusion proteins. In some such methods, the glycogen storage disease is Pompe disease. In some such methods, the Pompe disease is classic infantile-onset form Pompe disease. In some such methods, the Pompe disease is non-classic infantile form Pompe disease. In some such methods, the Pompe disease is late onset form Pompe disease. In some such methods, the subject has a GAA genotype selected from the group consisting of: ASP91ASN; MET318THR; GLU521LYS; GLY643ARG; ARG725TRP; IVS1AS, T-G, −13; LYS903DEL; LEU299ARG; SER529VAL; ASP645GLU; GLU689LYS; EX18DEL; PRO545LEU; 1-BP DEL, 525T; ARG854TER; ALA237VAL; GLY293ARG; and IVS6AS, G-C, −1.

In some such methods, the subject is administered the fusion protein in association with a further therapeutic agent. In some such methods, the further therapeutic agent is selected from: alglucosidase alfa, rituximab, methotrexate, intravenous immunoglobulin (IVIG), avalglucosidase alfa, levalbuterol, an antibiotic, cortisone, prednisone, a bisphosphonate, and palivizumab. In some such methods, the further therapeutic agent is selected from: a beta2-adrenergic agonist, a steroid, a bisphosphonate, an infectious disease treatment, a vaccine, and a pneumococcal vaccine. In some such methods, the subject is 1 year of age or less and experiences a symptom selected from:
trouble eating and not gaining weight;
poor head and neck control;
rolling over and sitting up later than expected;
breathing problems;
lung infection;
enlarged and thickening heart
heart defect;
enlarged liver; and
enlarged tongue.

In some such methods, the subject is an adult and experiences a symptom selected from:
weakness in the legs, trunk, and/or arms;
shortness of breath;
lung infection;
trouble breathing while sleeping;
spine curvature;
enlarged liver;
enlarged tongue; and
stiff joints.

In some such methods, one or more signs or symptoms of the GSD in the subject are alleviated after the fusion protein or antigen-binding protein is administered.

In another aspect, provided are methods for delivering a payload to a tissue or cell type in the body of a subject comprising administering, to the subject, an antigen-binding protein that binds specifically to transferrin receptor or an antigenic-fragment thereof or variant thereof to the subject fused to the payload. In some such methods, the payload is one or more antibodies or antigen-binding fragments thereof, proteins, enzymes or viral vectors containing one or more polynucleotides or oligonucleotides. In some such methods, the payload is human GAA protein or a variant thereof. In some such methods, the tissue is brain/spinal cord/CNS; eye; skeletal muscle; adipose tissue; blood/bone marrow; breast; lung/bronchus; colon; uterus; esophagus; heart; kidney; liver; lymph node; ovary; pancreas; placenta; prostate; rectum; skin; peripheral blood mononuclear cell (PBMC); small intestine; spleen; stomach; testis; peripheral nervous system; and/or bone/cartilage/joint. In some such methods, the cell type and tissue that is associate with the cell type is as follows:

| | |
|---|---|
| (1) brain/spinal cord/CNS tissue | endothelial cells |
| | neurons (all types) |
| | oligodendrocytes (and/or precursors) |
| | pericytes |
| | meninges/leptomeningeal cells |
| | arachnoid barrier cells |
| | peripheral glia |
| | astrocytes |
| | glia |
| | Schwann cells |
| | ependymal cells |
| | microglia; |
| (2) eye tissue | rod photoreceptor cells |
| | Muller glia cells |
| | bipolar cells |
| | cone photoreceptor cells |
| | endothelial cells |
| | cornea |
| | sclera |
| | optic nerve |
| | pupillary sphincter; |
| (3) skeletal muscle tissue | skeletal myocytes |
| | fibroblasts |
| | endothelial cells |
| | macrophages |
| | satellite cells; |
| (4) adipose tissue | adipocytes |
| | fibroblasts |
| | T-cells |
| | macrophages |
| | B-cells |
| | dendritic cells; |
| (5) blood/bone marrow tissue | T-cells |
| | B-cells |
| | macrophages |
| | erythroid cells |
| | plasmid cells |
| | dendritic cells; |
| (6) breast tissue | glandular cells |
| | T-cells |
| | fibroblasts |
| | macrophages |
| | endothelial cells |
| | myoepithelial cells |
| | adipocytes; |
| (7) lung/bronchus tissue | basal respiratory cells |
| | respiratory ciliated cells |
| | club cells |
| | smooth muscle cells |
| | ionocytes |
| | macrophages |
| | alveolar cells (type 1 and/or 2) |
| | T-cells |
| | endothelial cells; |
| (8) colon tissue | distal enterocytes |
| | intestinal goblet cells |
| | undifferentiated cells |
| | T-cells |
| | Paneth cells |
| | B-cells |
| | enteroendocrine cells; |
| (9) uterus tissue | glandular and luminal cells |
| | endometrial stromal cells |
| | endothelial cells |
| | smooth muscle cells |
| | T-cells |
| | macrophages; |
| (10) esophagus tissue | fibroblasts |
| | squamous epithelial cells |
| | endothelial cells |
| | smooth muscle cells |
| | macrophages |
| | plasma cells |
| | T-cells; |
| (11) heart tissue | cardiomyocytes |
| | endothelial cells |
| | fibroblasts |
| | macrophages |
| | T-cells |
| | B-cells |
| | dendritic cells; |
| (12) kidney tissue | proximal tubular cells |
| | T-cells |
| | macrophages |
| | collecting duct cells |
| | B-cells |
| | glomeruli |
| | fibroblasts; |
| (13) liver tissue | hepatocytes |
| | B-cells |
| | erythroid cells; |
| (14) lymph node tissue | B-cells |
| | T-cells; |
| (15) ovary tissue | granulosa cells |
| | fibroblasts |
| | smooth muscle cells |
| | macrophages |
| | T-cells |
| | theca cells |
| | fibroblasts; |
| (16) pancreas tissue | ductal cells |
| | pancreatic endocrine cells |
| | smooth muscle cells |
| | endothelial cells |
| | macrophages |
| | exocrine glandular cells |
| | monocytes; |
| (17) placenta tissue | cytotrophoblasts |
| | extravillous trophoblasts |
| | fibroblasts |
| | Hofbauer cells |
| | endothelial cells; |
| (18) prostate tissue | basal prostatic cells |
| | prostatic glandular cells |
| | urothelial cells |
| | endothelial cells |
| | fibroblasts |
| | smooth muscle cells |
| | macrophages |
| | T-cells; |
| (19) rectum tissue | undifferentiated cells |
| | intestinal goblet cells |
| | Paneth cells |
| | distal enterocytes |
| | enteroendocrine cells; |
| (20) skin tissue | Langerhans cells |
| | fibroblasts |
| | endothelial cells |
| | basal keratinocytes |
| | suprabasal keratinocytes |
| | T-cells |
| | smooth muscle cells |
| | melanocytes; |
| (21) PBMC tissue | monocytes |
| | T-cells |
| | NK-cells |
| | dendritic cells; |
| (22) small intestine tissue | proximal enterocytes |
| | undifferentiated cells |
| | intestinal goblet cells |
| | Paneth cells; |
| (23) spleen tissue | B-cells |
| | T-cells |
| | plasma cells |
| | macrophages; |
| (24) stomach tissue | B-cells |
| | T-cells |
| | gastric mucus-secreting cells |
| | plasma cells |
| | fibroblasts |
| | macrophages; |
| (25) testes tissue | Leydig cells |
| | late spermatids |
| | spermatogonia |
| | early spermatids |
| | macrophages |
| | spermatocytes |
| | peritubular cells |
| | Sertoli cells |
| | endothelial cells; |
| (26) peripheral nervous system | motor neurons |
| tissue | sensory neurons |
| | Schwann cells |
| | dorsal root ganglion; |
| (27) bone/cartilage/joint tissue | chondrocytes |
| | chondroblasts |
| | mesenchymal cells |
| | osteoblasts |
| | osteoclasts. |

In some such methods, the method comprises piercing the body of the subject with a needle of a syringe and injecting the antigen-binding protein that binds specifically to transferrin receptor or an antigenic-fragment thereof or variant thereof to the subject fused to the payload into the body of the subject. In some such methods, the subject suffers from a muscle atrophy condition, metabolic disease, sarcopenia or cachexia.

In another aspect, provided are methods of expressing in a cell a fusion protein comprising an antigen-binding protein that binds specifically to transferrin receptor or an antigenic-fragment thereof or variant thereof fused to a payload comprising: (a) administering to the cell a gene therapy vector comprising any of the above polynucleotides, wherein the isolated polynucleotide encodes the fusion protein; (b) allowing the isolated polynucleotide to integrate into a genomic locus of the cell; and (c) allowing the cell to produce the fusion protein.

In some such methods, the method further comprises administering a nuclease agent or one or more polynucleotides encoding the nuclease agent to the cell, wherein the nuclease agent cleaves a nuclease target site in the genomic locus, and the isolated polynucleotide is integrated into the genomic locus. In some such methods, the nuclease agent comprises a Clustered Regularly Interspersed Short Palindromic Repeats (CRISPR)/CRISPR-associated (Cas) system, a zinc finger nuclease (ZFN), or a Transcription Activator-Like Effector Nuclease (TALEN). In some such methods, the cell is in vivo in a subject. In some such methods, the cell is ex vivo. In some such methods, the gene therapy vector is a viral vector, a naked polynucleotide, or a polynucleotide complex, optionally wherein the polynucleotide complex is a lipid nanoparticle comprising the polynucleotide. In some such methods, the gene therapy vector is a viral vector selected from the group consisting of a retrovirus, an adenovirus, a herpes simplex virus, a pox virus, a vaccinia virus, a lentivirus, or an adeno-associated virus. In some such methods, the gene therapy vector is an adeno-associated virus (AAV) vector, optionally wherein the gene therapy vector is an AAV2/8 chimera and/or an AAV pseudotyped to the liver. In some such methods, the genomic locus is a safe harbor locus. In some such methods, the genomic locus is at or proximal to a locus selected from the group consisting of an EESYR locus, a SARS locus, position 188,083,272 of human chromosome 1 or its non-human mammalian orthologue, position 3,046,320 of human chromosome 10 or its non-human mammalian orthologue, position 67,328,980 of human chromosome 17 or its non-human mammalian orthologue, an adeno-associated virus site 1 (AAVS1) on chromosome, a naturally occurring site of integration of AAV virus on human chromosome 19 or its non-human mammalian orthologue, a chemokine receptor 5 (CCR5) gene, a chemokine receptor gene encoding an HIV-1 coreceptor, a mouse Rosa26 locus or its non-murine mammalian orthologue, and an albumin (alb) locus. In some such methods, the cell is a human cell. In some such methods, the cell is a liver cell.

Provided herein are antigen-binding proteins that can be fused to a payload having one or more of the following characteristics: (1) Affinity (K_{D}) for binding to human TfR at 25° C. in surface plasmon resonance format of about 41 nM or a higher affinity; (2) Affinity (K_{D}) for binding to monkey TfR at 25° C. in surface plasmon resonance format of about 0 nM (no detectable binding) or a higher affinity; (3) Ratio of [K_{D} for binding to monkey TfR/K_{D} for binding to human TfR] at 25° C. in surface plasmon resonance format of from 0 to 278; (4) Blocks about 3-13% hTfR binding to Human Holo-Tf when in Fab format (IgG1); (5) Blocks about 6-13% hTfR binding to Human Holo-Tf when in scFv (V_{K}-V_{H}) format; (6) Blocks about 11-26% hTfR binding to Human Holo-Tf when in scFv (V_{H}-V_{L}) format; (7) When comprising the antigen-binding protein fused to GAA, exhibits a ratio of about 1-2 mature hGAA protein in brain (normalized to that of positive control 8D3:GAA scFv) when administered to mice expressing human transferrin receptor (optionally, lacking functional endogenous GAA) when in anti-hTfR scFv:hGAA format; (8) When comprising the antigen-binding protein fused to GAA, exhibits a ratio of about 0.1-1.2 mature hGAA protein in brain parenchyma (normalized to that of positive control 8D3:GAA scFv) when administered to mice expressing human transferrin receptor (optionally, lacking functional endogenous GAA) when in anti-hTfR scFv:hGAA format; (9) When comprising the antigen-binding protein fused to GAA, exhibits a ratio of about 0.67, 1.80, 1.78 or 7.74 (about 1-2) mature hGAA protein in quadriceps (normalized to that of positive control 8D3:GAA scFv) when administered to mice expressing human transferrin receptor (optionally, lacking functional endogenous GAA) when in anti-hTfR scFv:hGAA format; (10) When comprising the antigen-binding protein fused to GAA, exhibits a ratio of about 0.1-1.2 mature hGAA protein in brain parenchyma (normalized to that of positive control 8D3:GAA scFv) when administered to mice expressing human transferrin receptor (optionally, lacking functional endogenous GAA) when in anti-hTfR scFv:hGAA format; (11) When comprising the antigen-binding protein fused to GAA, delivers mature hGAA protein to serum, liver, cerebrum, cerebellum, spinal cord, heart and/or quadricep when administered to mice expressing human transferrin receptor (optionally, lacking functional endogenous GAA) when in anti-hTfR scFv:hGAA format; (12) When comprising the antigen-binding protein fused to GAA, reduces glycogen stored in cerebrum, cerebellum, spinal cord, heart and/or quadricep when administered to mice expressing human transferrin receptor (optionally, lacking functional endogenous GAA) when in anti-hTfR scFv:hGAA format; (13) When comprising the antigen-binding protein fused to GAA, reduces glycogen levels in cerebellum of mice expressing human transferrin receptor but lacking functional endogenous GAA by at least about 90% relative to that of untreated mice; (14) When comprising the antigen-binding protein fused to GAA, reduces glycogen levels in quadricep of mice expressing human transferrin receptor but lacking functional endogenous GAA by at least about 89% relative to that of untreated mice; and/or (15) Does not cause abnormal iron homeostasis when administered to mice expressing human transferrin receptor.

Provided herein is an antibody or antigen-binding fragment thereof that binds specifically to transferrin receptor (e.g., human transferrin receptor) that comprises: (i) a heavy chain variable region that comprises the HCDR1, HCDR2 and HCDR3 of a HCVR comprising the amino acid sequence set forth in SEQ ID NO: 2; 462; 12; 463; 22; 464; 32; 42; 52; 467; 62; 492; 72; 470; 82; 92; 472; 102; 112; 473; 122; 132; 142; 475; 152; 162; 477; 172; 182; 478; 192; 480; 202; 481; 212; 222; 232; 242; 252; 482; 262; 272; 282; 292; 302; 483 or 312 (or a variant thereof); and/or (ii) a light chain variable region that comprises the LCDR1, LCDR2 and LCDR3 of a LCVR comprising the amino acid sequence set forth in SEQ ID NO: 7; 17; 27; 37; 465; 47; 466; 57; 468; 67; 469; 77; 471; 87; 97; 107; 117; 474; 127; 137; 147; 476; 157; 167; 177; 187; 479; 197; 207; 217; 227; 237; 247; 257; 267; 277; 287; 297; 307; 488; 317 or 484 (or a variant thereof). In an embodiment, the antibody or antigen-binding fragment thereof comprises: (1) a HCVR comprising the HCDR1, HCDR2 and HCDR3 of a HCVR that comprises the amino acid sequence set forth in SEQ ID NO: 2 (or a variant thereof); and a LCVR comprising the LCDR1, LCDR2 and LCDR3 of a LCVR that comprises the amino acid sequence set forth in SEQ ID NO: 7 (or a variant thereof); (2) a HCVR comprising the HCDR1, HCDR2 and HCDR3 of a HCVR that comprises the amino acid sequence set forth in SEQ ID NO: 12 (or a variant thereof); and a LCVR comprising the LCDR1, LCDR2 and LCDR3 of a LCVR that comprises the amino acid sequence set forth in SEQ ID NO: 17 (or a variant thereof); (3) a HCVR comprising the HCDR1, HCDR2 and HCDR3 of a HCVR that comprises the amino acid sequence set forth in SEQ ID NO: 22 (or a variant thereof); and a LCVR comprising the LCDR1, LCDR2 and LCDR3 of a LCVR that comprises the amino acid sequence set forth in SEQ ID NO: 27 (or a variant thereof); (4) a HCVR comprising the HCDR1, HCDR2 and HCDR3 of a HCVR that comprises the amino acid sequence set forth in SEQ ID NO: 32 (or a variant thereof); and a LCVR comprising the LCDR1, LCDR2 and LCDR3 of a LCVR that comprises the amino acid sequence set forth in SEQ ID NO: 37 (or a variant thereof); (5) a HCVR comprising the HCDR1, HCDR2 and HCDR3 of a HCVR that comprises the amino acid sequence set forth in SEQ ID NO: 42 (or a variant thereof); and a LCVR comprising the LCDR1, LCDR2 and LCDR3 of a LCVR that comprises the amino acid sequence set forth in SEQ ID NO: 47 (or a variant thereof); (6) a HCVR comprising the HCDR1, HCDR2 and HCDR3 of a HCVR that comprises the amino acid sequence set forth in SEQ ID NO: 52 (or a variant thereof); and a LCVR comprising the LCDR1, LCDR2 and LCDR3 of a LCVR that comprises the amino acid sequence set forth in SEQ ID NO: 57 (or a variant thereof); (7) a HCVR comprising the HCDR1, HCDR2 and HCDR3 of a HCVR that comprises the amino acid sequence set forth in SEQ ID NO: 62 (or a variant thereof); and a LCVR comprising the LCDR1, LCDR2 and LCDR3 of a LCVR that comprises the amino acid sequence set forth in SEQ ID NO: 67 (or a variant thereof); (8) a HCVR comprising the HCDR1, HCDR2 and HCDR3 of a HCVR that comprises the amino acid sequence set forth in SEQ ID NO: 72 (or a variant thereof); and a LCVR comprising the LCDR1, LCDR2 and LCDR3 of a LCVR that comprises the amino acid sequence set forth in SEQ ID NO: 77 (or a variant thereof); (9) a HCVR comprising the HCDR1, HCDR2 and HCDR3 of a HCVR that comprises the amino acid sequence set forth in SEQ ID NO: 82 (or a variant thereof); and a LCVR comprising the LCDR1, LCDR2 and LCDR3 of a LCVR that comprises the amino acid sequence set forth in SEQ ID NO: 87 (or a variant thereof); (10) a HCVR comprising the HCDR1, HCDR2 and HCDR3 of a HCVR that comprises the amino acid sequence set forth in SEQ ID NO: 92 (or a variant thereof); and a LCVR comprising the LCDR1, LCDR2 and LCDR3 of a LCVR that comprises the amino acid sequence set forth in SEQ ID NO: 97 (or a variant thereof); (11) a HCVR comprising the HCDR1, HCDR2 and HCDR3 of a HCVR that comprises the amino acid sequence set forth in SEQ ID NO: 102 (or a variant thereof); and a LCVR comprising the LCDR1, LCDR2 and LCDR3 of a LCVR that comprises the amino acid sequence set forth in SEQ ID NO: 107 (or a variant thereof); (12) a HCVR comprising the HCDR1, HCDR2 and HCDR3 of a HCVR that comprises the amino acid sequence set forth in SEQ ID NO: 112 (or a variant thereof); and a LCVR comprising the LCDR1, LCDR2 and LCDR3 of a LCVR that comprises the amino acid sequence set forth in SEQ ID NO: 117 (or a variant thereof); (13) a HCVR comprising the HCDR1, HCDR2 and HCDR3 of a HCVR that comprises the amino acid sequence set forth in SEQ ID NO: 122 (or a variant thereof); and a LCVR comprising the LCDR1, LCDR2 and LCDR3 of a LCVR that comprises the amino acid sequence set forth in SEQ ID NO: 127 (or a variant thereof); (14) a HCVR comprising the HCDR1, HCDR2 and HCDR3 of a HCVR that comprises the amino acid sequence set forth in SEQ ID NO: 132 (or a variant thereof); and a LCVR comprising the LCDR1, LCDR2 and LCDR3 of a LCVR that comprises the amino acid sequence set forth in SEQ ID NO: 137 (or a variant thereof); (15) a HCVR comprising the HCDR1, HCDR2 and HCDR3 of a HCVR that comprises the amino acid sequence set forth in SEQ ID NO: 142 (or a variant thereof); and a LCVR comprising the LCDR1, LCDR2 and LCDR3 of a LCVR that comprises the amino acid sequence set forth in SEQ ID NO: 147 (or a variant thereof); (16) a HCVR comprising the HCDR1, HCDR2 and HCDR3 of a HCVR that comprises the amino acid sequence set forth in SEQ ID NO: 152 (or a variant thereof); and a LCVR comprising the LCDR1, LCDR2 and LCDR3 of a LCVR that comprises the amino acid sequence set forth in SEQ ID NO: 157 (or a variant thereof); (17) a HCVR comprising the HCDR1, HCDR2 and HCDR3 of a HCVR that comprises the amino acid sequence set forth in SEQ ID NO: 162 (or a variant thereof); and a LCVR comprising the LCDR1, LCDR2 and LCDR3 of a LCVR that comprises the amino acid sequence set forth in SEQ ID NO: 167 (or a variant thereof); (18) a HCVR comprising the HCDR1, HCDR2 and HCDR3 of a HCVR that comprises the amino acid sequence set forth in SEQ ID NO: 172 (or a variant thereof); and a LCVR comprising the LCDR1, LCDR2 and LCDR3 of a LCVR that comprises the amino acid sequence set forth in SEQ ID NO: 177 (or a variant thereof); (19) a HCVR comprising the HCDR1, HCDR2 and HCDR3 of a HCVR that comprises the amino acid sequence set forth in SEQ ID NO: 182 (or a variant thereof); and a LCVR comprising the LCDR1, LCDR2 and LCDR3 of a LCVR that comprises the amino acid sequence set forth in SEQ ID NO: 187 (or a variant thereof); (20) a HCVR comprising the HCDR1, HCDR2 and HCDR3 of a HCVR that comprises the amino acid sequence set forth in SEQ ID NO: 192 (or a variant thereof); and a LCVR comprising the LCDR1, LCDR2 and LCDR3 of a LCVR that comprises the amino acid sequence set forth in SEQ ID NO: 197 (or a variant thereof); (21) a HCVR comprising the HCDR1, HCDR2 and HCDR3 of a HCVR that comprises the amino acid sequence set forth in SEQ ID NO: 202 (or a variant thereof); and a LCVR comprising the LCDR1, LCDR2 and LCDR3 of a LCVR that comprises the amino acid sequence set forth in SEQ ID NO: 207 (or a variant thereof); (22) a HCVR comprising the HCDR1, HCDR2 and HCDR3 of a HCVR that comprises the amino acid sequence set forth in SEQ ID NO: 212 (or a variant thereof); and a LCVR comprising the LCDR1, LCDR2 and LCDR3 of a LCVR that comprises the amino acid sequence set forth in SEQ ID NO: 217 (or a variant thereof); (23) a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof); (24) a HCVR comprising the HCDR1, HCDR2 and HCDR3 of a HCVR that comprises the amino acid sequence set forth in SEQ ID NO: 232 (or a variant thereof); and a LCVR comprising the LCDR1, LCDR2 and LCDR3 of a LCVR that comprises the amino acid sequence set forth in SEQ ID NO: 237 (or a variant thereof); (25) a HCVR comprising the HCDR1, HCDR2 and HCDR3 of a HCVR that comprises the amino acid sequence set forth in SEQ ID NO: 242 (or a variant thereof); and a LCVR comprising the LCDR1, LCDR2 and LCDR3 of a LCVR that comprises the amino acid sequence set forth in SEQ ID NO: 247 (or a variant thereof); (26) a HCVR comprising the HCDR1, HCDR2 and HCDR3 of a HCVR that comprises the amino acid sequence set forth in SEQ ID NO: 252 (or a variant thereof); and a LCVR comprising the LCDR1, LCDR2 and LCDR3 of a LCVR that comprises the amino acid sequence set forth in SEQ ID NO: 257 (or a variant thereof); (27) a HCVR comprising the HCDR1, HCDR2 and HCDR3 of a HCVR that comprises the amino acid sequence set forth in SEQ ID NO: 262 (or a variant thereof); and a LCVR comprising the LCDR1, LCDR2 and LCDR3 of a LCVR that comprises the amino acid sequence set forth in SEQ ID NO: 267 (or a variant thereof); (28) a HCVR comprising the HCDR1, HCDR2 and HCDR3 of a HCVR that comprises the amino acid sequence set forth in SEQ ID NO: 272 (or a variant thereof); and a LCVR comprising the LCDR1, LCDR2 and LCDR3 of a LCVR that comprises the amino acid sequence set forth in SEQ ID NO: 277 (or a variant thereof); (29) a HCVR comprising the HCDR1, HCDR2 and HCDR3 of a HCVR that comprises the amino acid sequence set forth in SEQ ID NO: 282 (or a variant thereof); and a LCVR comprising the LCDR1, LCDR2 and LCDR3 of a LCVR that comprises the amino acid sequence set forth in SEQ ID NO: 287 (or a variant thereof); (30) a HCVR comprising the HCDR1, HCDR2 and HCDR3 of a HCVR that comprises the amino acid sequence set forth in SEQ ID NO: 292 (or a variant thereof); and a LCVR comprising the LCDR1, LCDR2 and LCDR3 of a LCVR that comprises the amino acid sequence set forth in SEQ ID NO: 297 (or a variant thereof); (31) a HCVR comprising the HCDR1, HCDR2 and HCDR3 of a HCVR that comprises the amino acid sequence set forth in SEQ ID NO: 302 (or a variant thereof); and a LCVR comprising the LCDR1, LCDR2 and LCDR3 of a LCVR that comprises the amino acid sequence set forth in SEQ ID NO: 307 or 488 (or a variant thereof); and/or (32) a HCVR comprising the HCDR1, HCDR2 and HCDR3 of a HCVR that comprises the amino acid sequence set forth in SEQ ID NO: 312 (or a variant thereof); and a LCVR comprising the LCDR1, LCDR2 and LCDR3 of a LCVR that comprises the amino acid sequence set forth in SEQ ID NO: 317 (or a variant thereof). In an embodiment, the antibody or antigen-binding fragment thereof comprises: (23) a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof); or (25) a HCVR comprising the HCDR1, HCDR2 and HCDR3 of a HCVR that comprises the amino acid sequence set forth in SEQ ID NO: 242 (or a variant thereof); and a LCVR comprising the LCDR1, LCDR2 and LCDR3 of a LCVR that comprises the amino acid sequence set forth in SEQ ID NO: 247 (or a variant thereof). In an embodiment, the antibody or antigen-binding fragment thereof comprises: (23) a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof). In an embodiment, the antibody or antigen-binding fragment thereof comprises: (25) a HCVR comprising the HCDR1, HCDR2 and HCDR3 of a HCVR that comprises the amino acid sequence set forth in SEQ ID NO: 242 (or a variant thereof); and a LCVR comprising the LCDR1, LCDR2 and LCDR3 of a LCVR that comprises the amino acid sequence set forth in SEQ ID NO: 247 (or a variant thereof). In an embodiment, the antibody or antigen-binding fragment thereof comprises: (14) a HCVR comprising the HCDR1, HCDR2 and HCDR3 of a HCVR that comprises the amino acid sequence set forth in SEQ ID NO: 132 (or a variant thereof); and a LCVR comprising the LCDR1, LCDR2 and LCDR3 of a LCVR that comprises the amino acid sequence set forth in SEQ ID NO: 137 (or a variant thereof). In an embodiment, the antibody or antigen-binding fragment thereof comprises: (18) a HCVR comprising the HCDR1, HCDR2 and HCDR3 of a HCVR that comprises the amino acid sequence set forth in SEQ ID NO: 172 (or a variant thereof); and a LCVR comprising the LCDR1, LCDR2 and LCDR3 of a LCVR that comprises the amino acid sequence set forth in SEQ ID NO: 177 (or a variant thereof). In an embodiment, the antibody or antigen-binding fragment thereof comprises: (27) a HCVR comprising the HCDR1, HCDR2 and HCDR3 of a HCVR that comprises the amino acid sequence set forth in SEQ ID NO: 262 (or a variant thereof); and a LCVR comprising the LCDR1, LCDR2 and LCDR3 of a LCVR that comprises the amino acid sequence set forth in SEQ ID NO: 267 (or a variant thereof). In an embodiment, the antibody or antigen-binding fragment thereof comprises: (28) a HCVR comprising the HCDR1, HCDR2 and HCDR3 of a HCVR that comprises the amino acid sequence set forth in SEQ ID NO: 272 (or a variant thereof); and a LCVR comprising the LCDR1, LCDR2 and LCDR3 of a LCVR that comprises the amino acid sequence set forth in SEQ ID NO: 277 (or a variant thereof).

In an embodiment, the antibody or antigen-binding fragment comprises: (a) a HCVR that comprises: an HCDR1 comprising the amino acid sequence set forth in SEQ ID NO: 3 (or a variant thereof), an HCDR2 comprising the amino acid sequence set forth in SEQ ID NO: 4 (or a variant thereof), and an HCDR3 comprising the amino acid sequence set forth in SEQ ID NO: 5 (or a variant thereof); and a LCVR that comprises: an LCDR1 comprising the amino acid sequence set forth in SEQ ID NO: 8 (or a variant thereof), an LCDR2 comprising the amino acid sequence set forth in SEQ ID NO: 9 (or a variant thereof), and an LCDR3 comprising the amino acid sequence set forth in SEQ ID NO: 10 (or a variant thereof); (b) a HCVR that comprises: an HCDR1 comprising the amino acid sequence set forth in SEQ ID NO: 13 (or a variant thereof), an HCDR2 comprising the amino acid sequence set forth in SEQ ID NO: 14 (or a variant thereof), and an HCDR3 comprising the amino acid sequence set forth in SEQ ID NO: 15 (or a variant thereof); and a LCVR that comprises: an LCDR1 comprising the amino acid sequence set forth in SEQ ID NO: 18 (or a variant thereof), an LCDR2 comprising the amino acid sequence set forth in SEQ ID NO: 19 (or a variant thereof), and an LCDR3 comprising the amino acid sequence set forth in SEQ ID NO: 20 (or a variant thereof); (c) a HCVR that comprises: an HCDR1 comprising the amino acid sequence set forth in SEQ ID NO: 23 (or a variant thereof), an HCDR2 comprising the amino acid sequence set forth in SEQ ID NO: 24 (or a variant thereof), and an HCDR3 comprising the amino acid sequence set forth in SEQ ID NO: 25 (or a variant thereof); and a LCVR that comprises: an LCDR1 comprising the amino acid sequence set forth in SEQ ID NO: 28 (or a variant thereof), an LCDR2 comprising the amino acid sequence set forth in SEQ ID NO: 29 (or a variant thereof), and an LCDR3 comprising the amino acid sequence set forth in SEQ ID NO: 30 (or a variant thereof); (d) a HCVR that comprises: an HCDR1 comprising the amino acid sequence set forth in SEQ ID NO: 33 (or a variant thereof), an HCDR2 comprising the amino acid sequence set forth in SEQ ID NO: 34 (or a variant thereof), and an HCDR3 comprising the amino acid sequence set forth in SEQ ID NO: 35 (or a variant thereof); and a LCVR that comprises: an LCDR1 comprising the amino acid sequence set forth in SEQ ID NO: 38 (or a variant thereof), an LCDR2 comprising the amino acid sequence set forth in SEQ ID NO: 39 (or a variant thereof), and an LCDR3 comprising the amino acid sequence set forth in SEQ ID NO: 40 (or a variant thereof); (e) a HCVR that comprises: an HCDR1 comprising the amino acid sequence set forth in SEQ ID NO: 43 (or a variant thereof), an HCDR2 comprising the amino acid sequence set forth in SEQ ID NO: 44 (or a variant thereof), and an HCDR3 comprising the amino acid sequence set forth in SEQ ID NO: 45 (or a variant thereof); and a LCVR that comprises: an LCDR1 comprising the amino acid sequence set forth in SEQ ID NO: 48 (or a variant thereof), an LCDR2 comprising the amino acid sequence set forth in SEQ ID NO: 49 (or a variant thereof), and an LCDR3 comprising the amino acid sequence set forth in SEQ ID NO: 50 (or a variant thereof); (f) a HCVR that comprises: an HCDR1 comprising the amino acid sequence set forth in SEQ ID NO: 53 (or a variant thereof), an HCDR2 comprising the amino acid sequence set forth in SEQ ID NO: 54 (or a variant thereof), and an HCDR3 comprising the amino acid sequence set forth in SEQ ID NO: 55 (or a variant thereof); and a LCVR that comprises: an LCDR1 comprising the amino acid sequence set forth in SEQ ID NO: 58 (or a variant thereof), an LCDR2 comprising the amino acid sequence set forth in SEQ ID NO: 59 (or a variant thereof), and an LCDR3 comprising the amino acid sequence set forth in SEQ ID NO: 60 (or a variant thereof); (g) a HCVR that comprises: an HCDR1 comprising the amino acid sequence set forth in SEQ ID NO: 63 (or a variant thereof), an HCDR2 comprising the amino acid sequence set forth in SEQ ID NO: 64 (or a variant thereof), and an HCDR3 comprising the amino acid sequence set forth in SEQ ID NO: 65 (or a variant thereof); and a LCVR that comprises: an LCDR1 comprising the amino acid sequence set forth in SEQ ID NO: 68 (or a variant thereof), an LCDR2 comprising the amino acid sequence set forth in SEQ ID NO: 69 (or a variant thereof), and an LCDR3 comprising the amino acid sequence set forth in SEQ ID NO: 70 (or a variant thereof); (h) a HCVR that comprises: an HCDR1 comprising the amino acid sequence set forth in SEQ ID NO: 73 (or a variant thereof), an HCDR2 comprising the amino acid sequence set forth in SEQ ID NO: 74 (or a variant thereof), and an HCDR3 comprising the amino acid sequence set forth in SEQ ID NO: 75 (or a variant thereof); and a LCVR that comprises: an LCDR1 comprising the amino acid sequence set forth in SEQ ID NO: 78 (or a variant thereof), an LCDR2 comprising the amino acid sequence set forth in SEQ ID NO: 79 (or a variant thereof), and an LCDR3 comprising the amino acid sequence set forth in SEQ ID NO: 80 (or a variant thereof); (i) a HCVR that comprises: an HCDR1 comprising the amino acid sequence set forth in SEQ ID NO: 83 (or a variant thereof), an HCDR2 comprising the amino acid sequence set forth in SEQ ID NO: 84 (or a variant thereof), and an HCDR3 comprising the amino acid sequence set forth in SEQ ID NO: 85 (or a variant thereof); and a LCVR that comprises: an LCDR1 comprising the amino acid sequence set forth in SEQ ID NO: 88 (or a variant thereof), an LCDR2 comprising the amino acid sequence set forth in SEQ ID NO: 89 (or a variant thereof), and an LCDR3 comprising the amino acid sequence set forth in SEQ ID NO: 90 (or a variant thereof); (j) a HCVR that comprises: an HCDR1 comprising the amino acid sequence set forth in SEQ ID NO: 93 (or a variant thereof), an HCDR2 comprising the amino acid sequence set forth in SEQ ID NO: 94 (or a variant thereof), and an HCDR3 comprising the amino acid sequence set forth in SEQ ID NO: 95 (or a variant thereof); and a LCVR that comprises: an LCDR1 comprising the amino acid sequence set forth in SEQ ID NO: 98 (or a variant thereof), an LCDR2 comprising the amino acid sequence set forth in SEQ ID NO: 99 (or a variant thereof), and an LCDR3 comprising the amino acid sequence set forth in SEQ ID NO: 100 (or a variant thereof); (k) a HCVR that comprises: an HCDR1 comprising the amino acid sequence set forth in SEQ ID NO: 103 (or a variant thereof), an HCDR2 comprising the amino acid sequence set forth in SEQ ID NO: 104 (or a variant thereof), and an HCDR3 comprising the amino acid sequence set forth in SEQ ID NO: 105 (or a variant thereof); and a LCVR that comprises: an LCDR1 comprising the amino acid sequence set forth in SEQ ID NO: 108 (or a variant thereof), an LCDR2 comprising the amino acid sequence set forth in SEQ ID NO: 109 (or a variant thereof), and an LCDR3 comprising the amino acid sequence set forth in SEQ ID NO: 110 (or a variant thereof); (1) a HCVR that comprises: an HCDR1 comprising the amino acid sequence set forth in SEQ ID NO: 113 (or a variant thereof), an HCDR2 comprising the amino acid sequence set forth in SEQ ID NO: 114 (or a variant thereof), and an HCDR3 comprising the amino acid sequence set forth in SEQ ID NO: 115 (or a variant thereof); and a LCVR that comprises: an LCDR1 comprising the amino acid sequence set forth in SEQ ID NO: 118 (or a variant thereof), an LCDR2 comprising the amino acid sequence set forth in SEQ ID NO: 119 (or a variant thereof), and an LCDR3 comprising the amino acid sequence set forth in SEQ ID NO: 120 (or a variant thereof); (m) a HCVR that comprises: an HCDR1 comprising the amino acid sequence set forth in SEQ ID NO: 123 (or a variant thereof), an HCDR2 comprising the amino acid sequence set forth in SEQ ID NO: 124 (or a variant thereof), and an HCDR3 comprising the amino acid sequence set forth in SEQ ID NO: 125 (or a variant thereof); and a LCVR that comprises: an LCDR1 comprising the amino acid sequence set forth in SEQ ID NO: 128 (or a variant thereof), an LCDR2 comprising the amino acid sequence set forth in SEQ ID NO: 129 (or a variant thereof), and an LCDR3 comprising the amino acid sequence set forth in SEQ ID NO: 130 (or a variant thereof); (n) a HCVR that comprises: an HCDR1 comprising the amino acid sequence set forth in SEQ ID NO: 133 (or a variant thereof), an HCDR2 comprising the amino acid sequence set forth in SEQ ID NO: 134 (or a variant thereof), and an HCDR3 comprising the amino acid sequence set forth in SEQ ID NO: 135 (or a variant thereof); and a LCVR that comprises: an LCDR1 comprising the amino acid sequence set forth in SEQ ID NO: 138 (or a variant thereof), an LCDR2 comprising the amino acid sequence set forth in SEQ ID NO: 139 (or a variant thereof), and an LCDR3 comprising the amino acid sequence set forth in SEQ ID NO: 140 (or a variant thereof); (o) a HCVR that comprises: an HCDR1 comprising the amino acid sequence set forth in SEQ ID NO: 143 (or a variant thereof), an HCDR2 comprising the amino acid sequence set forth in SEQ ID NO: 144 (or a variant thereof), and an HCDR3 comprising the amino acid sequence set forth in SEQ ID NO: 145 (or a variant thereof); and a LCVR that comprises: an LCDR1 comprising the amino acid sequence set forth in SEQ ID NO: 148 (or a variant thereof), an LCDR2 comprising the amino acid sequence set forth in SEQ ID NO: 149 (or a variant thereof), and an LCDR3 comprising the amino acid sequence set forth in SEQ ID NO: 150 (or a variant thereof); (p) a HCVR that comprises: an HCDR1 comprising the amino acid sequence set forth in SEQ ID NO: 153 (or a variant thereof), an HCDR2 comprising the amino acid sequence set forth in SEQ ID NO: 154 (or a variant thereof), and an HCDR3 comprising the amino acid sequence set forth in SEQ ID NO: 155 (or a variant thereof); and a LCVR that comprises: an LCDR1 comprising the amino acid sequence set forth in SEQ ID NO: 158 (or a variant thereof), an LCDR2 comprising the amino acid sequence set forth in SEQ ID NO: 159 (or a variant thereof), and an LCDR3 comprising the amino acid sequence set forth in SEQ ID NO: 160 (or a variant thereof); (q) a HCVR that comprises: an HCDR1 comprising the amino acid sequence set forth in SEQ ID NO: 163 (or a variant thereof), an HCDR2 comprising the amino acid sequence set forth in SEQ ID NO: 164 (or a variant thereof), and an HCDR3 comprising the amino acid sequence set forth in SEQ ID NO: 165 (or a variant thereof); and a LCVR that comprises: an LCDR1 comprising the amino acid sequence set forth in SEQ ID NO: 168 (or a variant thereof), an LCDR2 comprising the amino acid sequence set forth in SEQ ID NO: 169 (or a variant thereof), and an LCDR3 comprising the amino acid sequence set forth in SEQ ID NO: 170 (or a variant thereof); (r) a HCVR that comprises: an HCDR1 comprising the amino acid sequence set forth in SEQ ID NO: 173 (or a variant thereof), an HCDR2 comprising the amino acid sequence set forth in SEQ ID NO: 174 (or a variant thereof), and an HCDR3 comprising the amino acid sequence set forth in SEQ ID NO: 175 (or a variant thereof); and a LCVR that comprises: an LCDR1 comprising the amino acid sequence set forth in SEQ ID NO: 178 (or a variant thereof), an LCDR2 comprising the amino acid sequence set forth in SEQ ID NO: 179 (or a variant thereof), and an LCDR3 comprising the amino acid sequence set forth in SEQ ID NO: 180 (or a variant thereof); (s) a HCVR that comprises: an HCDR1 comprising the amino acid sequence set forth in SEQ ID NO: 183 (or a variant thereof), an HCDR2 comprising the amino acid sequence set forth in SEQ ID NO: 184 (or a variant thereof), and an HCDR3 comprising the amino acid sequence set forth in SEQ ID NO: 185 (or a variant thereof); and a LCVR that comprises: an LCDR1 comprising the amino acid sequence set forth in SEQ ID NO: 188 (or a variant thereof), an LCDR2 comprising the amino acid sequence set forth in SEQ ID NO: 189 (or a variant thereof), and an LCDR3 comprising the amino acid sequence set forth in SEQ ID NO: 190 (or a variant thereof); (t) a HCVR that comprises: an HCDR1 comprising the amino acid sequence set forth in SEQ ID NO: 193 (or a variant thereof), an HCDR2 comprising the amino acid sequence set forth in SEQ ID NO: 194 (or a variant thereof), and an HCDR3 comprising the amino acid sequence set forth in SEQ ID NO: 195 (or a variant thereof); and a LCVR that comprises: an LCDR1 comprising the amino acid sequence set forth in SEQ ID NO: 198 (or a variant thereof), an LCDR2 comprising the amino acid sequence set forth in SEQ ID NO: 199 (or a variant thereof), and an LCDR3 comprising the amino acid sequence set forth in SEQ ID NO: 200 (or a variant thereof); (u) a HCVR that comprises: an HCDR1 comprising the amino acid sequence set forth in SEQ ID NO: 203 (or a variant thereof), an HCDR2 comprising the amino acid sequence set forth in SEQ ID NO: 204 (or a variant thereof), and an HCDR3 comprising the amino acid sequence set forth in SEQ ID NO: 205 (or a variant thereof); and a LCVR that comprises: an LCDR1 comprising the amino acid sequence set forth in SEQ ID NO: 208 (or a variant thereof), an LCDR2 comprising the amino acid sequence set forth in SEQ ID NO: 209 (or a variant thereof), and an LCDR3 comprising the amino acid sequence set forth in SEQ ID NO: 210 (or a variant thereof); (v) a HCVR that comprises: an HCDR1 comprising the amino acid sequence set forth in SEQ ID NO: 213 (or a variant thereof), an HCDR2 comprising the amino acid sequence set forth in SEQ ID NO: 214 (or a variant thereof), and an HCDR3 comprising the amino acid sequence set forth in SEQ ID NO: 215 (or a variant thereof); and a LCVR that comprises: an LCDR1 comprising the amino acid sequence set forth in SEQ ID NO: 218 (or a variant thereof), an LCDR2 comprising the amino acid sequence set forth in SEQ ID NO: 219 (or a variant thereof), and an LCDR3 comprising the amino acid sequence set forth in SEQ ID NO: 220 (or a variant thereof); (w) a HCVR that comprises: an HCDR1 comprising the amino acid sequence set forth in SEQ ID NO: 223 (or a variant thereof), an HCDR2 comprising the amino acid sequence set forth in SEQ ID NO: 224 (or a variant thereof), and an HCDR3 comprising the amino acid sequence set forth in SEQ ID NO: 225 (or a variant thereof); and a LCVR that comprises: an LCDR1 comprising the amino acid sequence set forth in SEQ ID NO: 228 (or a variant thereof), an LCDR2 comprising the amino acid sequence set forth in SEQ ID NO: 229 (or a variant thereof), and an LCDR3 comprising the amino acid sequence set forth in SEQ ID NO: 230 (or a variant thereof); (x) a HCVR that comprises: an HCDR1 comprising the amino acid sequence set forth in SEQ ID NO: 233 (or a variant thereof), an HCDR2 comprising the amino acid sequence set forth in SEQ ID NO: 234 (or a variant thereof), and an HCDR3 comprising the amino acid sequence set forth in SEQ ID NO: 235 (or a variant thereof); and a LCVR that comprises: an LCDR1 comprising the amino acid sequence set forth in SEQ ID NO: 238 (or a variant thereof), an LCDR2 comprising the amino acid sequence set forth in SEQ ID NO: 239 (or a variant thereof), and an LCDR3 comprising the amino acid sequence set forth in SEQ ID NO: 240 (or a variant thereof); (y) a HCVR that comprises: an HCDR1 comprising the amino acid sequence set forth in SEQ ID NO: 243 (or a variant thereof), an HCDR2 comprising the amino acid sequence set forth in SEQ ID NO: 244 (or a variant thereof), and an HCDR3 comprising the amino acid sequence set forth in SEQ ID NO: 245 (or a variant thereof); and a LCVR that comprises: an LCDR1 comprising the amino acid sequence set forth in SEQ ID NO: 248 (or a variant thereof), an LCDR2 comprising the amino acid sequence set forth in SEQ ID NO: 249 (or a variant thereof), and an LCDR3 comprising the amino acid sequence set forth in SEQ ID NO: 250 (or a variant thereof); (z) a HCVR that comprises: an HCDR1 comprising the amino acid sequence set forth in SEQ ID NO: 253 (or a variant thereof), an HCDR2 comprising the amino acid sequence set forth in SEQ ID NO: 254 (or a variant thereof), and an HCDR3 comprising the amino acid sequence set forth in SEQ ID NO: 255 (or a variant thereof); and a LCVR that comprises: an LCDR1 comprising the amino acid sequence set forth in SEQ ID NO: 258 (or a variant thereof), an LCDR2 comprising the amino acid sequence set forth in SEQ ID NO: 259 (or a variant thereof), and an LCDR3 comprising the amino acid sequence set forth in SEQ ID NO: 260 (or a variant thereof); (aa) a HCVR that comprises: an HCDR1 comprising the amino acid sequence set forth in SEQ ID NO: 263 (or a variant thereof), an HCDR2 comprising the amino acid sequence set forth in SEQ ID NO: 264 (or a variant thereof), and an HCDR3 comprising the amino acid sequence set forth in SEQ ID NO: 265 (or a variant thereof); and a LCVR that comprises: an LCDR1 comprising the amino acid sequence set forth in SEQ ID NO: 268 (or a variant thereof), an LCDR2 comprising the amino acid sequence set forth in SEQ ID NO: 269 (or a variant thereof), and an LCDR3 comprising the amino acid sequence set forth in SEQ ID NO: 270 (or a variant thereof); (ab) a HCVR that comprises: an HCDR1 comprising the amino acid sequence set forth in SEQ ID NO: 273 (or a variant thereof), an HCDR2 comprising the amino acid sequence set forth in SEQ ID NO: 274 (or a variant thereof), and an HCDR3 comprising the amino acid sequence set forth in SEQ ID NO: 275 (or a variant thereof); and a LCVR that comprises: an LCDR1 comprising the amino acid sequence set forth in SEQ ID NO: 278 (or a variant thereof), an LCDR2 comprising the amino acid sequence set forth in SEQ ID NO: 279 (or a variant thereof), and an LCDR3 comprising the amino acid sequence set forth in SEQ ID NO: 280 (or a variant thereof); (ac) a HCVR that comprises: an HCDR1 comprising the amino acid sequence set forth in SEQ ID NO: 283 (or a variant thereof), an HCDR2 comprising the amino acid sequence set forth in SEQ ID NO: 284 (or a variant thereof), and an HCDR3 comprising the amino acid sequence set forth in SEQ ID NO: 285 (or a variant thereof); and a LCVR that comprises: an LCDR1 comprising the amino acid sequence set forth in SEQ ID NO: 288 (or a variant thereof), an LCDR2 comprising the amino acid sequence set forth in SEQ ID NO: 289 (or a variant thereof), and an LCDR3 comprising the amino acid sequence set forth in SEQ ID NO: 290 (or a variant thereof); (ad) a HCVR that comprises: an HCDR1 comprising the amino acid sequence set forth in SEQ ID NO: 293 (or a variant thereof), an HCDR2 comprising the amino acid sequence set forth in SEQ ID NO: 294 (or a variant thereof), and an HCDR3 comprising the amino acid sequence set forth in SEQ ID NO: 295 (or a variant thereof); and a LCVR that comprises: an LCDR1 comprising the amino acid sequence set forth in SEQ ID NO: 298 (or a variant thereof), an LCDR2 comprising the amino acid sequence set forth in SEQ ID NO: 299 (or a variant thereof), and an LCDR3 comprising the amino acid sequence set forth in SEQ ID NO: 300 (or a variant thereof); (ae) a HCVR that comprises: an HCDR1 comprising the amino acid sequence set forth in SEQ ID NO: 303 (or a variant thereof), an HCDR2 comprising the amino acid sequence set forth in SEQ ID NO: 304 (or a variant thereof), and an HCDR3 comprising the amino acid sequence set forth in SEQ ID NO: 305 (or a variant thereof); and a LCVR that comprises: an LCDR1 comprising the amino acid sequence set forth in SEQ ID NO: 308 (or a variant thereof), an LCDR2 comprising the amino acid sequence set forth in SEQ ID NO: 309 (or a variant thereof), and an LCDR3 comprising the amino acid sequence set forth in SEQ ID NO: 310 (or a variant thereof); and/or (af) a HCVR that comprises: an HCDR1 comprising the amino acid sequence set forth in SEQ ID NO: 313 (or a variant thereof), an HCDR2 comprising the amino acid sequence set forth in SEQ ID NO: 314 (or a variant thereof), and an HCDR3 comprising the amino acid sequence set forth in SEQ ID NO: 315 (or a variant thereof); and a LCVR that comprises: an LCDR1 comprising the amino acid sequence set forth in SEQ ID NO: 318 (or a variant thereof), an LCDR2 comprising the amino acid sequence set forth in SEQ ID NO: 319 (or a variant thereof), and an LCDR3 comprising the amino acid sequence set forth in SEQ ID NO: 320 (or a variant thereof). In an embodiment, the antibody or antigen-binding fragment comprises: (w) a HCVR that comprises: an HCDR1 comprising the amino acid sequence set forth in SEQ ID NO: 223 (or a variant thereof), an HCDR2 comprising the amino acid sequence set forth in SEQ ID NO: 224 (or a variant thereof), and an HCDR3 comprising the amino acid sequence set forth in SEQ ID NO: 225 (or a variant thereof); and a LCVR that comprises: an LCDR1 comprising the amino acid sequence set forth in SEQ ID NO: 228 (or a variant thereof), an LCDR2 comprising the amino acid sequence set forth in SEQ ID NO: 229 (or a variant thereof), and an LCDR3 comprising the amino acid sequence set forth in SEQ ID NO: 230 (or a variant thereof); or (y) a HCVR that comprises: an HCDR1 comprising the amino acid sequence set forth in SEQ ID NO: 243 (or a variant thereof), an HCDR2 comprising the amino acid sequence set forth in SEQ ID NO: 244 (or a variant thereof), and an HCDR3 comprising the amino acid sequence set forth in SEQ ID NO: 245 (or a variant thereof); and a LCVR that comprises: an LCDR1 comprising the amino acid sequence set forth in SEQ ID NO: 248 (or a variant thereof), an LCDR2 comprising the amino acid sequence set forth in SEQ ID NO: 249 (or a variant thereof), and an LCDR3 comprising the amino acid sequence set forth in SEQ ID NO: 250 (or a variant thereof. In an embodiment, the antibody or antigen-binding fragment comprises: (w) a HCVR that comprises: an HCDR1 comprising the amino acid sequence set forth in SEQ ID NO: 223 (or a variant thereof), an HCDR2 comprising the amino acid sequence set forth in SEQ ID NO: 224 (or a variant thereof), and an HCDR3 comprising the amino acid sequence set forth in SEQ ID NO: 225 (or a variant thereof); and a LCVR that comprises: an LCDR1 comprising the amino acid sequence set forth in SEQ ID NO: 228 (or a variant thereof), an LCDR2 comprising the amino acid sequence set forth in SEQ ID NO: 229 (or a variant thereof), and an LCDR3 comprising the amino acid sequence set forth in SEQ ID NO: 230 (or a variant thereof). In an embodiment, the antibody or antigen-binding fragment comprises: (y) a HCVR that comprises: an HCDR1 comprising the amino acid sequence set forth in SEQ ID NO: 243 (or a variant thereof), an HCDR2 comprising the amino acid sequence set forth in SEQ ID NO: 244 (or a variant thereof), and an HCDR3 comprising the amino acid sequence set forth in SEQ ID NO: 245 (or a variant thereof); and a LCVR that comprises: an LCDR1 comprising the amino acid sequence set forth in SEQ ID NO: 248 (or a variant thereof), an LCDR2 comprising the amino acid sequence set forth in SEQ ID NO: 249 (or a variant thereof), and an LCDR3 comprising the amino acid sequence set forth in SEQ ID NO: 250 (or a variant thereof. In an embodiment, the antibody or antigen-binding fragment comprises: (n) a HCVR that comprises: an HCDR1 comprising the amino acid sequence set forth in SEQ ID NO: 133 (or a variant thereof), an HCDR2 comprising the amino acid sequence set forth in SEQ ID NO: 134 (or a variant thereof), and an HCDR3 comprising the amino acid sequence set forth in SEQ ID NO: 135 (or a variant thereof); and a LCVR that comprises: an LCDR1 comprising the amino acid sequence set forth in SEQ ID NO: 138 (or a variant thereof), an LCDR2 comprising the amino acid sequence set forth in SEQ ID NO: 139 (or a variant thereof), and an LCDR3 comprising the amino acid sequence set forth in SEQ ID NO: 140 (or a variant thereof). In an embodiment, the antibody or antigen-binding fragment comprises: (r) a HCVR that comprises: an HCDR1 comprising the amino acid sequence set forth in SEQ ID NO: 173 (or a variant thereof), an HCDR2 comprising the amino acid sequence set forth in SEQ ID NO: 174 (or a variant thereof), and an HCDR3 comprising the amino acid sequence set forth in SEQ ID NO: 175 (or a variant thereof); and a LCVR that comprises: an LCDR1 comprising the amino acid sequence set forth in SEQ ID NO: 178 (or a variant thereof), an LCDR2 comprising the amino acid sequence set forth in SEQ ID NO: 179 (or a variant thereof), and an LCDR3 comprising the amino acid sequence set forth in SEQ ID NO: 180 (or a variant thereof). In an embodiment, the antibody or antigen-binding fragment comprises: (aa) a HCVR that comprises: an HCDR1 comprising the amino acid sequence set forth in SEQ ID NO: 263 (or a variant thereof), an HCDR2 comprising the amino acid sequence set forth in SEQ ID NO: 264 (or a variant thereof), and an HCDR3 comprising the amino acid sequence set forth in SEQ ID NO: 265 (or a variant thereof); and a LCVR that comprises: an LCDR1 comprising the amino acid sequence set forth in SEQ ID NO: 268 (or a variant thereof), an LCDR2 comprising the amino acid sequence set forth in SEQ ID NO: 269 (or a variant thereof), and an LCDR3 comprising the amino acid sequence set forth in SEQ ID NO: 270 (or a variant thereof). In an embodiment, the antibody or antigen-binding fragment comprises: (ab) a HCVR that comprises: an HCDR1 comprising the amino acid sequence set forth in SEQ ID NO: 273 (or a variant thereof), an HCDR2 comprising the amino acid sequence set forth in SEQ ID NO: 274 (or a variant thereof), and an HCDR3 comprising the amino acid sequence set forth in SEQ ID NO: 275 (or a variant thereof); and a LCVR that comprises: an LCDR1 comprising the amino acid sequence set forth in SEQ ID NO: 278 (or a variant thereof), an LCDR2 comprising the amino acid sequence set forth in SEQ ID NO: 279 (or a variant thereof), and an LCDR3 comprising the amino acid sequence set forth in SEQ ID NO: 280 (or a variant thereof).

In an embodiment, the antibody or antigen-binding fragment comprises: (i) a HCVR that comprises the amino acid sequence set forth in SEQ ID NO: 2 (or a variant thereof); and a LCVR that comprises the amino acid sequence set forth in SEQ ID NO: 7 (or a variant thereof); (ii) a HCVR that comprises the amino acid sequence set forth in SEQ ID NO: 12 (or a variant thereof); and a LCVR that comprises the amino acid sequence set forth in SEQ ID NO: 17 (or a variant thereof); (iii) a HCVR that comprises the amino acid sequence set forth in SEQ ID NO: 22 (or a variant thereof); and a LCVR that comprises the amino acid sequence set forth in SEQ ID NO: 27 (or a variant thereof); (iv) a HCVR that comprises the amino acid sequence set forth in SEQ ID NO: 32 (or a variant thereof); and a LCVR that comprises the amino acid sequence set forth in SEQ ID NO: 37 (or a variant thereof); (v) a HCVR that comprises the amino acid sequence set forth in SEQ ID NO: 42 (or a variant thereof); and a LCVR that comprises the amino acid sequence set forth in SEQ ID NO: 47 (or a variant thereof); (vi) a HCVR that comprises the amino acid sequence set forth in SEQ ID NO: 52 (or a variant thereof); and a LCVR that comprises the amino acid sequence set forth in SEQ ID NO: 57 (or a variant thereof); (vii) a HCVR that comprises the amino acid sequence set forth in SEQ ID NO: 62 (or a variant thereof); and a LCVR that comprises the amino acid sequence set forth in SEQ ID NO: 67 (or a variant thereof); (viii) a HCVR that comprises the amino acid sequence set forth in SEQ ID NO: 72 (or a variant thereof); and a LCVR that comprises the amino acid sequence set forth in SEQ ID NO: 77 (or a variant thereof); (ix) a HCVR that comprises the amino acid sequence set forth in SEQ ID NO: 82 (or a variant thereof); and a LCVR that comprises the amino acid sequence set forth in SEQ ID NO: 87 (or a variant thereof); (x) a HCVR that comprises the amino acid sequence set forth in SEQ ID NO: 92 (or a variant thereof); and a LCVR that comprises the amino acid sequence set forth in SEQ ID NO: 97 (or a variant thereof); (xi) a HCVR that comprises the amino acid sequence set forth in SEQ ID NO: 102 (or a variant thereof); and a LCVR that comprises the amino acid sequence set forth in SEQ ID NO: 107 (or a variant thereof); (xii) a HCVR that comprises the amino acid sequence set forth in SEQ ID NO: 112 (or a variant thereof); and a LCVR that comprises the amino acid sequence set forth in SEQ ID NO: 117 (or a variant thereof); (xiii) a HCVR that comprises the amino acid sequence set forth in SEQ ID NO: 122 (or a variant thereof); and a LCVR that comprises the amino acid sequence set forth in SEQ ID NO: 127 (or a variant thereof); (xiv) a HCVR that comprises the amino acid sequence set forth in SEQ ID NO: 132 (or a variant thereof); and a LCVR that comprises the amino acid sequence set forth in SEQ ID NO: 137 (or a variant thereof); (xv) a HCVR that comprises the amino acid sequence set forth in SEQ ID NO: 142 (or a variant thereof); and a LCVR that comprises the amino acid sequence set forth in SEQ ID NO: 147 (or a variant thereof); (xvi) a HCVR that comprises the amino acid sequence set forth in SEQ ID NO: 152 (or a variant thereof); and a LCVR that comprises the amino acid sequence set forth in SEQ ID NO: 157 (or a variant thereof); (xvii) a HCVR that comprises the amino acid sequence set forth in SEQ ID NO: 162 (or a variant thereof); and a LCVR that comprises the amino acid sequence set forth in SEQ ID NO: 167 (or a variant thereof); (xviii) a HCVR that comprises the amino acid sequence set forth in SEQ ID NO: 172 (or a variant thereof); and a LCVR that comprises the amino acid sequence set forth in SEQ ID NO: 177 (or a variant thereof); (xix) a HCVR that comprises the amino acid sequence set forth in SEQ ID NO: 182 (or a variant thereof); and a LCVR that comprises the amino acid sequence set forth in SEQ ID NO: 187 (or a variant thereof); (xx) a HCVR that comprises the amino acid sequence set forth in SEQ ID NO: 192 (or a variant thereof); and a LCVR that comprises the amino acid sequence set forth in SEQ ID NO: 197 (or a variant thereof); (xxi) a HCVR that comprises the amino acid sequence set forth in SEQ ID NO: 202 (or a variant thereof); and a LCVR that comprises the amino acid sequence set forth in SEQ ID NO: 207 (or a variant thereof); (xxii) a HCVR that comprises the amino acid sequence set forth in SEQ ID NO: 212 (or a variant thereof); and a LCVR that comprises the amino acid sequence set forth in SEQ ID NO: 217 (or a variant thereof); (xxiii) a HCVR that comprises the amino acid sequence set forth in SEQ ID NO: 222 (or a variant thereof); and a LCVR that comprises the amino acid sequence set forth in SEQ ID NO: 227 (or a variant thereof); (xxiv) a HCVR that comprises the amino acid sequence set forth in SEQ ID NO: 232 (or a variant thereof); and a LCVR that comprises the amino acid sequence set forth in SEQ ID NO: 237 (or a variant thereof); (xxv) a HCVR that comprises the amino acid sequence set forth in SEQ ID NO: 242 (or a variant thereof); and a LCVR that comprises the amino acid sequence set forth in SEQ ID NO: 247 (or a variant thereof); (xxvi) a HCVR that comprises the amino acid sequence set forth in SEQ ID NO: 252 (or a variant thereof); and a LCVR that comprises the amino acid sequence set forth in SEQ ID NO: 257 (or a variant thereof); (xxvii) a HCVR that comprises the amino acid sequence set forth in SEQ ID NO: 262 (or a variant thereof); and a LCVR that comprises the amino acid sequence set forth in SEQ ID NO: 267 (or a variant thereof); (xxviii) a HCVR that comprises the amino acid sequence set forth in SEQ ID NO: 272 (or a variant thereof); and a LCVR that comprises the amino acid sequence set forth in SEQ ID NO: 277 (or a variant thereof); (xxix) a HCVR that comprises the amino acid sequence set forth in SEQ ID NO: 282 (or a variant thereof); and a LCVR that comprises the amino acid sequence set forth in SEQ ID NO: 287 (or a variant thereof); (xxx) a HCVR that comprises the amino acid sequence set forth in SEQ ID NO: 292 (or a variant thereof); and a LCVR that comprises the amino acid sequence set forth in SEQ ID NO: 297 (or a variant thereof); (xxxi) a HCVR that comprises the amino acid sequence set forth in SEQ ID NO: 302 (or a variant thereof); and a LCVR that comprises the amino acid sequence set forth in SEQ ID NO: 307 or 488 (or a variant thereof); and/or (xxxii) a HCVR that comprises the amino acid sequence set forth in SEQ ID NO: 312 (or a variant thereof); and a LCVR that comprises the amino acid sequence set forth in SEQ ID NO: 317 (or a variant thereof). See, e.g., FIG. 1. In an embodiment, the antibody or antigen-binding fragment comprises: (xxiii) a HCVR that comprises the amino acid sequence set forth in SEQ ID NO: 222 (or a variant thereof); and a LCVR that comprises the amino acid sequence set forth in SEQ ID NO: 227 (or a variant thereof); or (xxv) a HCVR that comprises the amino acid sequence set forth in SEQ ID NO: 242 (or a variant thereof); and a LCVR that comprises the amino acid sequence set forth in SEQ ID NO: 247 (or a variant thereof). In an embodiment, the antibody or antigen-binding fragment comprises: (xxiii) a HCVR that comprises the amino acid sequence set forth in SEQ ID NO: 222 (or a variant thereof); and a LCVR that comprises the amino acid sequence set forth in SEQ ID NO: 227 (or a variant thereof). In an embodiment, the antibody or antigen-binding fragment comprises: (xxv) a HCVR that comprises the amino acid sequence set forth in SEQ ID NO: 242 (or a variant thereof); and a LCVR that comprises the amino acid sequence set forth in SEQ ID NO: 247 (or a variant thereof). In an embodiment, the antibody or antigen-binding fragment comprises: (xiv) a HCVR that comprises the amino acid sequence set forth in SEQ ID NO: 132 (or a variant thereof); and a LCVR that comprises the amino acid sequence set forth in SEQ ID NO: 137 (or a variant thereof). In an embodiment, the antibody or antigen-binding fragment comprises: (xviii) a HCVR that comprises the amino acid sequence set forth in SEQ ID NO: 172 (or a variant thereof); and a LCVR that comprises the amino acid sequence set forth in SEQ ID NO: 177 (or a variant thereof). In an embodiment, the antibody or antigen-binding fragment comprises: (xxvii) a HCVR that comprises the amino acid sequence set forth in SEQ ID NO: 262 (or a variant thereof); and a LCVR that comprises the amino acid sequence set forth in SEQ ID NO: 267 (or a variant thereof). In an embodiment, the antibody or antigen-binding fragment comprises: (xxviii) a HCVR that comprises the amino acid sequence set forth in SEQ ID NO: 272 (or a variant thereof); and a LCVR that comprises the amino acid sequence set forth in SEQ ID NO: 277 (or a variant thereof).

In an embodiment, the antigen-binding fragment comprises an scFv. In an embodiment, the scFv comprises the amino acid sequence set forth in SEQ ID NO: 443 (or a variant thereof) or comprises the amino acid sequence set forth in SEQ ID NO: 440 (or a variant thereof). In an embodiment, the scFv comprises the amino acid sequence set forth in SEQ ID NO: 443 (or a variant thereof). In an embodiment, the antigen-binding fragment comprises an scFv. In an embodiment, the scFv comprises the amino acid sequence set forth in SEQ ID NO: 440 (or a variant thereof). In an embodiment, the scFv comprises the amino acid sequence set forth in SEQ ID NO: 429 (or a variant thereof). In an embodiment, the scFv comprises the amino acid sequence set forth in SEQ ID NO: 433 (or a variant thereof). In an embodiment, the scFv comprises the amino acid sequence set forth in SEQ ID NO: 442 (or a variant thereof). In an embodiment, the scFv comprises the amino acid sequence set forth in SEQ ID NO: 438 (or a variant thereof).

Provided herein is an anti-hTfR:Payload fusion protein comprising a single chain fragment variable (scFv), an antibody (e.g., an IgG, e.g., IgG1, IgG2, IgG3 or IgG4) or an antigen-binding fragment thereof (e.g., a Fab) that binds specifically to human transferrin receptor (or a vessel (e.g., vial) or injection device (e.g., syringe) containing such a fusion) (e.g., that binds to human transferrin receptor with a K_{D} of about 1×10⁻⁷ M or a greater affinity), which comprises a heavy chain variable region (V_{H}) and a light chain variable region (V_{L}), which is fused to a payload such as an alpha-glucosidase polypeptide (GAA), wherein a Fab having said V_{H} and V_{L} binds to human transferrin receptor with a K_{D} of about 0.65 nM or a greater affinity; and wherein, when said fusion protein is an anti-hTfR:GAA and is administered to a mouse expressing human transferrin receptor in the brain, the mouse achieves a molar ratio of mature GAA protein in the brain:serum GAA protein, in the mouse, of about 1:1 or greater when normalized against said ratio in mouse expressing mouse transferrin receptor that was administered 8D3. For example, where, when the fusion protein is an scfV, the scFv comprises domains arranged in the following orientation: N-Heavy chain variable region-Light chain variable region-GAA protein-C or N-Light chain variable region-Heavy chain variable region-Payload protein-C (“N-” denotes the amino terminus of the polypeptide and “C-” denotes the carboxy terminus of the polypeptide). In an embodiment, the scFv and the payload, e.g., GAA, are connected by a peptide linker such as -(GGGGS)ₘ- (SEQ ID NO: 426); wherein m is 1, 2, 3, 4, 5, 6, 7, 8, 9 or 10. In an embodiment, the scFv variable regions are connected by a peptide linker such as -(GGGGS)ₙ- (SEQ ID NO: 426); wherein n is 1, 2, 3, 4, 5, 6, 7, 8, 9 or 10.

In an embodiment, the fusion protein comprises the amino acid sequence set forth in SEQ ID NO: 408 (or a variant thereof) or comprises the amino acid sequence set forth in SEQ ID NO: 405 (or a variant thereof). In an embodiment, the fusion protein comprises the amino acid sequence set forth in SEQ ID NO: 408 (or a variant thereof). In an embodiment, the fusion protein comprises the amino acid sequence set forth in SEQ ID NO: 405 (or a variant thereof).

In an embodiment, the anti-hTfR:Payload fusion protein comprises: (i) a heavy chain variable region that comprises the HCDR1, HCDR2 and HCDR3 of a HCVR comprising the amino acid sequence set forth in SEQ ID NO: 2; 462; 12; 463; 22; 464; 32; 42; 52; 467; 62; 492; 72; 470; 82; 92; 472; 102; 112; 473; 122; 132; 142; 475; 152; 162; 477; 172; 182; 478; 192; 480; 202; 481; 212; 222; 232; 242; 252; 482; 262; 272; 282; 292; 302; 483 or 312 (or a variant thereof); and/or (ii) a light chain variable region that comprises the LCDR1, LCDR2 and LCDR3 of a LCVR comprising the amino acid sequence set forth in SEQ ID NO: 7; 17; 27; 37; 465; 47; 466; 57; 468; 67; 469; 77; 471; 87; 97; 107; 117; 474; 127; 137; 147; 476; 157; 167; 177; 187; 479; 197; 207; 217; 227; 237; 247; 257; 267; 277; 287; 297; 307; 488; 317 or 484 (or a variant thereof). In an embodiment, the fusion protein comprises: (1) a HCVR comprising the HCDR1, HCDR2 and HCDR3 of a HCVR that comprises the amino acid sequence set forth in SEQ ID NO: 2 (or a variant thereof); and a LCVR comprising the LCDR1, LCDR2 and LCDR3 of a LCVR that comprises the amino acid sequence set forth in SEQ ID NO: 7 (or a variant thereof); (2) a HCVR comprising the HCDR1, HCDR2 and HCDR3 of a HCVR that comprises the amino acid sequence set forth in SEQ ID NO: 12 (or a variant thereof); and a LCVR comprising the LCDR1, LCDR2 and LCDR3 of a LCVR that comprises the amino acid sequence set forth in SEQ ID NO: 17 (or a variant thereof); (3) a HCVR comprising the HCDR1, HCDR2 and HCDR3 of a HCVR that comprises the amino acid sequence set forth in SEQ ID NO: 22 (or a variant thereof); and a LCVR comprising the LCDR1, LCDR2 and LCDR3 of a LCVR that comprises the amino acid sequence set forth in SEQ ID NO: 27 (or a variant thereof); (4) a HCVR comprising the HCDR1, HCDR2 and HCDR3 of a HCVR that comprises the amino acid sequence set forth in SEQ ID NO: 32 (or a variant thereof); and a LCVR comprising the LCDR1, LCDR2 and LCDR3 of a LCVR that comprises the amino acid sequence set forth in SEQ ID NO: 37 (or a variant thereof); (5) a HCVR comprising the HCDR1, HCDR2 and HCDR3 of a HCVR that comprises the amino acid sequence set forth in SEQ ID NO: 42 (or a variant thereof); and a LCVR comprising the LCDR1, LCDR2 and LCDR3 of a LCVR that comprises the amino acid sequence set forth in SEQ ID NO: 47 (or a variant thereof); (6) a HCVR comprising the HCDR1, HCDR2 and HCDR3 of a HCVR that comprises the amino acid sequence set forth in SEQ ID NO: 52 (or a variant thereof); and a LCVR comprising the LCDR1, LCDR2 and LCDR3 of a LCVR that comprises the amino acid sequence set forth in SEQ ID NO: 57 (or a variant thereof); (7) a HCVR comprising the HCDR1, HCDR2 and HCDR3 of a HCVR that comprises the amino acid sequence set forth in SEQ ID NO: 62 (or a variant thereof); and a LCVR comprising the LCDR1, LCDR2 and LCDR3 of a LCVR that comprises the amino acid sequence set forth in SEQ ID NO: 67 (or a variant thereof); (8) a HCVR comprising the HCDR1, HCDR2 and HCDR3 of a HCVR that comprises the amino acid sequence set forth in SEQ ID NO: 72 (or a variant thereof); and a LCVR comprising the LCDR1, LCDR2 and LCDR3 of a LCVR that comprises the amino acid sequence set forth in SEQ ID NO: 77 (or a variant thereof); (9) a HCVR comprising the HCDR1, HCDR2 and HCDR3 of a HCVR that comprises the amino acid sequence set forth in SEQ ID NO: 82 (or a variant thereof); and a LCVR comprising the LCDR1, LCDR2 and LCDR3 of a LCVR that comprises the amino acid sequence set forth in SEQ ID NO: 87 (or a variant thereof); (10) a HCVR comprising the HCDR1, HCDR2 and HCDR3 of a HCVR that comprises the amino acid sequence set forth in SEQ ID NO: 92 (or a variant thereof); and a LCVR comprising the LCDR1, LCDR2 and LCDR3 of a LCVR that comprises the amino acid sequence set forth in SEQ ID NO: 97 (or a variant thereof); (11) a HCVR comprising the HCDR1, HCDR2 and HCDR3 of a HCVR that comprises the amino acid sequence set forth in SEQ ID NO: 102 (or a variant thereof); and a LCVR comprising the LCDR1, LCDR2 and LCDR3 of a LCVR that comprises the amino acid sequence set forth in SEQ ID NO: 107 (or a variant thereof); (12) a HCVR comprising the HCDR1, HCDR2 and HCDR3 of a HCVR that comprises the amino acid sequence set forth in SEQ ID NO: 112 (or a variant thereof); and a LCVR comprising the LCDR1, LCDR2 and LCDR3 of a LCVR that comprises the amino acid sequence set forth in SEQ ID NO: 117 (or a variant thereof); (13) a HCVR comprising the HCDR1, HCDR2 and HCDR3 of a HCVR that comprises the amino acid sequence set forth in SEQ ID NO: 122 (or a variant thereof); and a LCVR comprising the LCDR1, LCDR2 and LCDR3 of a LCVR that comprises the amino acid sequence set forth in SEQ ID NO: 127 (or a variant thereof); (14) a HCVR comprising the HCDR1, HCDR2 and HCDR3 of a HCVR that comprises the amino acid sequence set forth in SEQ ID NO: 132 (or a variant thereof); and a LCVR comprising the LCDR1, LCDR2 and LCDR3 of a LCVR that comprises the amino acid sequence set forth in SEQ ID NO: 137 (or a variant thereof); (15) a HCVR comprising the HCDR1, HCDR2 and HCDR3 of a HCVR that comprises the amino acid sequence set forth in SEQ ID NO: 142 (or a variant thereof); and a LCVR comprising the LCDR1, LCDR2 and LCDR3 of a LCVR that comprises the amino acid sequence set forth in SEQ ID NO: 147 (or a variant thereof); (16) a HCVR comprising the HCDR1, HCDR2 and HCDR3 of a HCVR that comprises the amino acid sequence set forth in SEQ ID NO: 152 (or a variant thereof); and a LCVR comprising the LCDR1, LCDR2 and LCDR3 of a LCVR that comprises the amino acid sequence set forth in SEQ ID NO: 157 (or a variant thereof); (17) a HCVR comprising the HCDR1, HCDR2 and HCDR3 of a HCVR that comprises the amino acid sequence set forth in SEQ ID NO: 162 (or a variant thereof); and a LCVR comprising the LCDR1, LCDR2 and LCDR3 of a LCVR that comprises the amino acid sequence set forth in SEQ ID NO: 167 (or a variant thereof); (18) a HCVR comprising the HCDR1, HCDR2 and HCDR3 of a HCVR that comprises the amino acid sequence set forth in SEQ ID NO: 172 (or a variant thereof); and a LCVR comprising the LCDR1, LCDR2 and LCDR3 of a LCVR that comprises the amino acid sequence set forth in SEQ ID NO: 177 (or a variant thereof); (19) a HCVR comprising the HCDR1, HCDR2 and HCDR3 of a HCVR that comprises the amino acid sequence set forth in SEQ ID NO: 182 (or a variant thereof); and a LCVR comprising the LCDR1, LCDR2 and LCDR3 of a LCVR that comprises the amino acid sequence set forth in SEQ ID NO: 187 (or a variant thereof); (20) a HCVR comprising the HCDR1, HCDR2 and HCDR3 of a HCVR that comprises the amino acid sequence set forth in SEQ ID NO: 192 (or a variant thereof); and a LCVR comprising the LCDR1, LCDR2 and LCDR3 of a LCVR that comprises the amino acid sequence set forth in SEQ ID NO: 197 (or a variant thereof); (21) a HCVR comprising the HCDR1, HCDR2 and HCDR3 of a HCVR that comprises the amino acid sequence set forth in SEQ ID NO: 202 (or a variant thereof); and a LCVR comprising the LCDR1, LCDR2 and LCDR3 of a LCVR that comprises the amino acid sequence set forth in SEQ ID NO: 207 (or a variant thereof); (22) a HCVR comprising the HCDR1, HCDR2 and HCDR3 of a HCVR that comprises the amino acid sequence set forth in SEQ ID NO: 212 (or a variant thereof); and a LCVR comprising the LCDR1, LCDR2 and LCDR3 of a LCVR that comprises the amino acid sequence set forth in SEQ ID NO: 217 (or a variant thereof); (23) a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof); (24) a HCVR comprising the HCDR1, HCDR2 and HCDR3 of a HCVR that comprises the amino acid sequence set forth in SEQ ID NO: 232 (or a variant thereof); and a LCVR comprising the LCDR1, LCDR2 and LCDR3 of a LCVR that comprises the amino acid sequence set forth in SEQ ID NO: 237 (or a variant thereof); (25) a HCVR comprising the HCDR1, HCDR2 and HCDR3 of a HCVR that comprises the amino acid sequence set forth in SEQ ID NO: 242 (or a variant thereof); and a LCVR comprising the LCDR1, LCDR2 and LCDR3 of a LCVR that comprises the amino acid sequence set forth in SEQ ID NO: 247 (or a variant thereof); (26) a HCVR comprising the HCDR1, HCDR2 and HCDR3 of a HCVR that comprises the amino acid sequence set forth in SEQ ID NO: 252 (or a variant thereof); and a LCVR comprising the LCDR1, LCDR2 and LCDR3 of a LCVR that comprises the amino acid sequence set forth in SEQ ID NO: 257 (or a variant thereof); (27) a HCVR comprising the HCDR1, HCDR2 and HCDR3 of a HCVR that comprises the amino acid sequence set forth in SEQ ID NO: 262 (or a variant thereof); and a LCVR comprising the LCDR1, LCDR2 and LCDR3 of a LCVR that comprises the amino acid sequence set forth in SEQ ID NO: 267 (or a variant thereof); (28) a HCVR comprising the HCDR1, HCDR2 and HCDR3 of a HCVR that comprises the amino acid sequence set forth in SEQ ID NO: 272 (or a variant thereof); and a LCVR comprising the LCDR1, LCDR2 and LCDR3 of a LCVR that comprises the amino acid sequence set forth in SEQ ID NO: 277 (or a variant thereof); (29) a HCVR comprising the HCDR1, HCDR2 and HCDR3 of a HCVR that comprises the amino acid sequence set forth in SEQ ID NO: 282 (or a variant thereof); and a LCVR comprising the LCDR1, LCDR2 and LCDR3 of a LCVR that comprises the amino acid sequence set forth in SEQ ID NO: 287 (or a variant thereof); (30) a HCVR comprising the HCDR1, HCDR2 and HCDR3 of a HCVR that comprises the amino acid sequence set forth in SEQ ID NO: 292 (or a variant thereof); and a LCVR comprising the LCDR1, LCDR2 and LCDR3 of a LCVR that comprises the amino acid sequence set forth in SEQ ID NO: 297 (or a variant thereof); (31) a HCVR comprising the HCDR1, HCDR2 and HCDR3 of a HCVR that comprises the amino acid sequence set forth in SEQ ID NO: 302 (or a variant thereof); and a LCVR comprising the LCDR1, LCDR2 and LCDR3 of a LCVR that comprises the amino acid sequence set forth in SEQ ID NO: 307 or 488 (or a variant thereof); and/or (32) a HCVR comprising the HCDR1, HCDR2 and HCDR3 of a HCVR that comprises the amino acid sequence set forth in SEQ ID NO: 312 (or a variant thereof); and a LCVR comprising the LCDR1, LCDR2 and LCDR3 of a LCVR that comprises the amino acid sequence set forth in SEQ ID NO: 317 (or a variant thereof). In an embodiment, the fusion protein comprises: (23) a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof); or (25) a HCVR comprising the HCDR1, HCDR2 and HCDR3 of a HCVR that comprises the amino acid sequence set forth in SEQ ID NO: 242 (or a variant thereof); and a LCVR comprising the LCDR1, LCDR2 and LCDR3 of a LCVR that comprises the amino acid sequence set forth in SEQ ID NO: 247 (or a variant thereof). In an embodiment, the fusion protein comprises: (23) a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof). In an embodiment, the fusion protein comprises: (25) a HCVR comprising the HCDR1, HCDR2 and HCDR3 of a HCVR that comprises the amino acid sequence set forth in SEQ ID NO: 242 (or a variant thereof); and a LCVR comprising the LCDR1, LCDR2 and LCDR3 of a LCVR that comprises the amino acid sequence set forth in SEQ ID NO: 247 (or a variant thereof). In an embodiment, the fusion protein comprises: (14) a HCVR comprising the HCDR1, HCDR2 and HCDR3 of a HCVR that comprises the amino acid sequence set forth in SEQ ID NO: 132 (or a variant thereof); and a LCVR comprising the LCDR1, LCDR2 and LCDR3 of a LCVR that comprises the amino acid sequence set forth in SEQ ID NO: 137 (or a variant thereof). In an embodiment, the fusion protein comprises: (18) a HCVR comprising the HCDR1, HCDR2 and HCDR3 of a HCVR that comprises the amino acid sequence set forth in SEQ ID NO: 172 (or a variant thereof); and a LCVR comprising the LCDR1, LCDR2 and LCDR3 of a LCVR that comprises the amino acid sequence set forth in SEQ ID NO: 177 (or a variant thereof). In an embodiment, the fusion protein comprises: (27) a HCVR comprising the HCDR1, HCDR2 and HCDR3 of a HCVR that comprises the amino acid sequence set forth in SEQ ID NO: 262 (or a variant thereof); and a LCVR comprising the LCDR1, LCDR2 and LCDR3 of a LCVR that comprises the amino acid sequence set forth in SEQ ID NO: 267 (or a variant thereof). In an embodiment, the fusion protein comprises: (28) a HCVR comprising the HCDR1, HCDR2 and HCDR3 of a HCVR that comprises the amino acid sequence set forth in SEQ ID NO: 272 (or a variant thereof); and a LCVR comprising the LCDR1, LCDR2 and LCDR3 of a LCVR that comprises the amino acid sequence set forth in SEQ ID NO: 277 (or a variant thereof).

In an embodiment, the fusion protein comprises: (a) a HCVR that comprises: an HCDR1 comprising the amino acid sequence set forth in SEQ ID NO: 3 (or a variant thereof), an HCDR2 comprising the amino acid sequence set forth in SEQ ID NO: 4 (or a variant thereof), and an HCDR3 comprising the amino acid sequence set forth in SEQ ID NO: 5 (or a variant thereof); and a LCVR that comprises: an LCDR1 comprising the amino acid sequence set forth in SEQ ID NO: 8 (or a variant thereof), an LCDR2 comprising the amino acid sequence set forth in SEQ ID NO: 9 (or a variant thereof), and an LCDR3 comprising the amino acid sequence set forth in SEQ ID NO: 10 (or a variant thereof); (b) a HCVR that comprises: an HCDR1 comprising the amino acid sequence set forth in SEQ ID NO: 13 (or a variant thereof), an HCDR2 comprising the amino acid sequence set forth in SEQ ID NO: 14 (or a variant thereof), and an HCDR3 comprising the amino acid sequence set forth in SEQ ID NO: 15 (or a variant thereof); and a LCVR that comprises: an LCDR1 comprising the amino acid sequence set forth in SEQ ID NO: 18 (or a variant thereof), an LCDR2 comprising the amino acid sequence set forth in SEQ ID NO: 19 (or a variant thereof), and an LCDR3 comprising the amino acid sequence set forth in SEQ ID NO: 20 (or a variant thereof); (c) a HCVR that comprises: an HCDR1 comprising the amino acid sequence set forth in SEQ ID NO: 23 (or a variant thereof), an HCDR2 comprising the amino acid sequence set forth in SEQ ID NO: 24 (or a variant thereof), and an HCDR3 comprising the amino acid sequence set forth in SEQ ID NO: 25 (or a variant thereof); and a LCVR that comprises: an LCDR1 comprising the amino acid sequence set forth in SEQ ID NO: 28 (or a variant thereof), an LCDR2 comprising the amino acid sequence set forth in SEQ ID NO: 29 (or a variant thereof), and an LCDR3 comprising the amino acid sequence set forth in SEQ ID NO: 30 (or a variant thereof); (d) a HCVR that comprises: an HCDR1 comprising the amino acid sequence set forth in SEQ ID NO: 33 (or a variant thereof), an HCDR2 comprising the amino acid sequence set forth in SEQ ID NO: 34 (or a variant thereof), and an HCDR3 comprising the amino acid sequence set forth in SEQ ID NO: 35 (or a variant thereof); and a LCVR that comprises: an LCDR1 comprising the amino acid sequence set forth in SEQ ID NO: 38 (or a variant thereof), an LCDR2 comprising the amino acid sequence set forth in SEQ ID NO: 39 (or a variant thereof), and an LCDR3 comprising the amino acid sequence set forth in SEQ ID NO: 40 (or a variant thereof); (e) a HCVR that comprises: an HCDR1 comprising the amino acid sequence set forth in SEQ ID NO: 43 (or a variant thereof), an HCDR2 comprising the amino acid sequence set forth in SEQ ID NO: 44 (or a variant thereof), and an HCDR3 comprising the amino acid sequence set forth in SEQ ID NO: 45 (or a variant thereof); and a LCVR that comprises: an LCDR1 comprising the amino acid sequence set forth in SEQ ID NO: 48 (or a variant thereof), an LCDR2 comprising the amino acid sequence set forth in SEQ ID NO: 49 (or a variant thereof), and an LCDR3 comprising the amino acid sequence set forth in SEQ ID NO: 50 (or a variant thereof); (f) a HCVR that comprises: an HCDR1 comprising the amino acid sequence set forth in SEQ ID NO: 53 (or a variant thereof), an HCDR2 comprising the amino acid sequence set forth in SEQ ID NO: 54 (or a variant thereof), and an HCDR3 comprising the amino acid sequence set forth in SEQ ID NO: 55 (or a variant thereof); and a LCVR that comprises: an LCDR1 comprising the amino acid sequence set forth in SEQ ID NO: 58 (or a variant thereof), an LCDR2 comprising the amino acid sequence set forth in SEQ ID NO: 59 (or a variant thereof), and an LCDR3 comprising the amino acid sequence set forth in SEQ ID NO: 60 (or a variant thereof); (g) a HCVR that comprises: an HCDR1 comprising the amino acid sequence set forth in SEQ ID NO: 63 (or a variant thereof), an HCDR2 comprising the amino acid sequence set forth in SEQ ID NO: 64 (or a variant thereof), and an HCDR3 comprising the amino acid sequence set forth in SEQ ID NO: 65 (or a variant thereof); and a LCVR that comprises: an LCDR1 comprising the amino acid sequence set forth in SEQ ID NO: 68 (or a variant thereof), an LCDR2 comprising the amino acid sequence set forth in SEQ ID NO: 69 (or a variant thereof), and an LCDR3 comprising the amino acid sequence set forth in SEQ ID NO: 70 (or a variant thereof); (h) a HCVR that comprises: an HCDR1 comprising the amino acid sequence set forth in SEQ ID NO: 73 (or a variant thereof), an HCDR2 comprising the amino acid sequence set forth in SEQ ID NO: 74 (or a variant thereof), and an HCDR3 comprising the amino acid sequence set forth in SEQ ID NO: 75 (or a variant thereof); and a LCVR that comprises: an LCDR1 comprising the amino acid sequence set forth in SEQ ID NO: 78 (or a variant thereof), an LCDR2 comprising the amino acid sequence set forth in SEQ ID NO: 79 (or a variant thereof), and an LCDR3 comprising the amino acid sequence set forth in SEQ ID NO: 80 (or a variant thereof); (i) a HCVR that comprises: an HCDR1 comprising the amino acid sequence set forth in SEQ ID NO: 83 (or a variant thereof), an HCDR2 comprising the amino acid sequence set forth in SEQ ID NO: 84 (or a variant thereof), and an HCDR3 comprising the amino acid sequence set forth in SEQ ID NO: 85 (or a variant thereof); and a LCVR that comprises: an LCDR1 comprising the amino acid sequence set forth in SEQ ID NO: 88 (or a variant thereof), an LCDR2 comprising the amino acid sequence set forth in SEQ ID NO: 89 (or a variant thereof), and an LCDR3 comprising the amino acid sequence set forth in SEQ ID NO: 90 (or a variant thereof); (j) a HCVR that comprises: an HCDR1 comprising the amino acid sequence set forth in SEQ ID NO: 93 (or a variant thereof), an HCDR2 comprising the amino acid sequence set forth in SEQ ID NO: 94 (or a variant thereof), and an HCDR3 comprising the amino acid sequence set forth in SEQ ID NO: 95 (or a variant thereof); and a LCVR that comprises: an LCDR1 comprising the amino acid sequence set forth in SEQ ID NO: 98 (or a variant thereof), an LCDR2 comprising the amino acid sequence set forth in SEQ ID NO: 99 (or a variant thereof), and an LCDR3 comprising the amino acid sequence set forth in SEQ ID NO: 100 (or a variant thereof); (k) a HCVR that comprises: an HCDR1 comprising the amino acid sequence set forth in SEQ ID NO: 103 (or a variant thereof), an HCDR2 comprising the amino acid sequence set forth in SEQ ID NO: 104 (or a variant thereof), and an HCDR3 comprising the amino acid sequence set forth in SEQ ID NO: 105 (or a variant thereof); and a LCVR that comprises: an LCDR1 comprising the amino acid sequence set forth in SEQ ID NO: 108 (or a variant thereof), an LCDR2 comprising the amino acid sequence set forth in SEQ ID NO: 109 (or a variant thereof), and an LCDR3 comprising the amino acid sequence set forth in SEQ ID NO: 110 (or a variant thereof); (1) a HCVR that comprises: an HCDR1 comprising the amino acid sequence set forth in SEQ ID NO: 113 (or a variant thereof), an HCDR2 comprising the amino acid sequence set forth in SEQ ID NO: 114 (or a variant thereof), and an HCDR3 comprising the amino acid sequence set forth in SEQ ID NO: 115 (or a variant thereof); and a LCVR that comprises: an LCDR1 comprising the amino acid sequence set forth in SEQ ID NO: 118 (or a variant thereof), an LCDR2 comprising the amino acid sequence set forth in SEQ ID NO: 119 (or a variant thereof), and an LCDR3 comprising the amino acid sequence set forth in SEQ ID NO: 120 (or a variant thereof); (m) a HCVR that comprises: an HCDR1 comprising the amino acid sequence set forth in SEQ ID NO: 123 (or a variant thereof), an HCDR2 comprising the amino acid sequence set forth in SEQ ID NO: 124 (or a variant thereof), and an HCDR3 comprising the amino acid sequence set forth in SEQ ID NO: 125 (or a variant thereof); and a LCVR that comprises: an LCDR1 comprising the amino acid sequence set forth in SEQ ID NO: 128 (or a variant thereof), an LCDR2 comprising the amino acid sequence set forth in SEQ ID NO: 129 (or a variant thereof), and an LCDR3 comprising the amino acid sequence set forth in SEQ ID NO: 130 (or a variant thereof); (n) a HCVR that comprises: an HCDR1 comprising the amino acid sequence set forth in SEQ ID NO: 133 (or a variant thereof), an HCDR2 comprising the amino acid sequence set forth in SEQ ID NO: 134 (or a variant thereof), and an HCDR3 comprising the amino acid sequence set forth in SEQ ID NO: 135 (or a variant thereof); and a LCVR that comprises: an LCDR1 comprising the amino acid sequence set forth in SEQ ID NO: 138 (or a variant thereof), an LCDR2 comprising the amino acid sequence set forth in SEQ ID NO: 139 (or a variant thereof), and an LCDR3 comprising the amino acid sequence set forth in SEQ ID NO: 140 (or a variant thereof); (o) a HCVR that comprises: an HCDR1 comprising the amino acid sequence set forth in SEQ ID NO: 143 (or a variant thereof), an HCDR2 comprising the amino acid sequence set forth in SEQ ID NO: 144 (or a variant thereof), and an HCDR3 comprising the amino acid sequence set forth in SEQ ID NO: 145 (or a variant thereof); and a LCVR that comprises: an LCDR1 comprising the amino acid sequence set forth in SEQ ID NO: 148 (or a variant thereof), an LCDR2 comprising the amino acid sequence set forth in SEQ ID NO: 149 (or a variant thereof), and an LCDR3 comprising the amino acid sequence set forth in SEQ ID NO: 150 (or a variant thereof); (p) a HCVR that comprises: an HCDR1 comprising the amino acid sequence set forth in SEQ ID NO: 153 (or a variant thereof), an HCDR2 comprising the amino acid sequence set forth in SEQ ID NO: 154 (or a variant thereof), and an HCDR3 comprising the amino acid sequence set forth in SEQ ID NO: 155 (or a variant thereof); and a LCVR that comprises: an LCDR1 comprising the amino acid sequence set forth in SEQ ID NO: 158 (or a variant thereof), an LCDR2 comprising the amino acid sequence set forth in SEQ ID NO: 159 (or a variant thereof), and an LCDR3 comprising the amino acid sequence set forth in SEQ ID NO: 160 (or a variant thereof); (q) a HCVR that comprises: an HCDR1 comprising the amino acid sequence set forth in SEQ ID NO: 163 (or a variant thereof), an HCDR2 comprising the amino acid sequence set forth in SEQ ID NO: 164 (or a variant thereof), and an HCDR3 comprising the amino acid sequence set forth in SEQ ID NO: 165 (or a variant thereof); and a LCVR that comprises: an LCDR1 comprising the amino acid sequence set forth in SEQ ID NO: 168 (or a variant thereof), an LCDR2 comprising the amino acid sequence set forth in SEQ ID NO: 169 (or a variant thereof), and an LCDR3 comprising the amino acid sequence set forth in SEQ ID NO: 170 (or a variant thereof); (r) a HCVR that comprises: an HCDR1 comprising the amino acid sequence set forth in SEQ ID NO: 173 (or a variant thereof), an HCDR2 comprising the amino acid sequence set forth in SEQ ID NO: 174 (or a variant thereof), and an HCDR3 comprising the amino acid sequence set forth in SEQ ID NO: 175 (or a variant thereof); and a LCVR that comprises: an LCDR1 comprising the amino acid sequence set forth in SEQ ID NO: 178 (or a variant thereof), an LCDR2 comprising the amino acid sequence set forth in SEQ ID NO: 179 (or a variant thereof), and an LCDR3 comprising the amino acid sequence set forth in SEQ ID NO: 180 (or a variant thereof); (s) a HCVR that comprises: an HCDR1 comprising the amino acid sequence set forth in SEQ ID NO: 183 (or a variant thereof), an HCDR2 comprising the amino acid sequence set forth in SEQ ID NO: 184 (or a variant thereof), and an HCDR3 comprising the amino acid sequence set forth in SEQ ID NO: 185 (or a variant thereof); and a LCVR that comprises: an LCDR1 comprising the amino acid sequence set forth in SEQ ID NO: 188 (or a variant thereof), an LCDR2 comprising the amino acid sequence set forth in SEQ ID NO: 189 (or a variant thereof), and an LCDR3 comprising the amino acid sequence set forth in SEQ ID NO: 190 (or a variant thereof); (t) a HCVR that comprises: an HCDR1 comprising the amino acid sequence set forth in SEQ ID NO: 193 (or a variant thereof), an HCDR2 comprising the amino acid sequence set forth in SEQ ID NO: 194 (or a variant thereof), and an HCDR3 comprising the amino acid sequence set forth in SEQ ID NO: 195 (or a variant thereof); and a LCVR that comprises: an LCDR1 comprising the amino acid sequence set forth in SEQ ID NO: 198 (or a variant thereof), an LCDR2 comprising the amino acid sequence set forth in SEQ ID NO: 199 (or a variant thereof), and an LCDR3 comprising the amino acid sequence set forth in SEQ ID NO: 200 (or a variant thereof); (u) a HCVR that comprises: an HCDR1 comprising the amino acid sequence set forth in SEQ ID NO: 203 (or a variant thereof), an HCDR2 comprising the amino acid sequence set forth in SEQ ID NO: 204 (or a variant thereof), and an HCDR3 comprising the amino acid sequence set forth in SEQ ID NO: 205 (or a variant thereof); and a LCVR that comprises: an LCDR1 comprising the amino acid sequence set forth in SEQ ID NO: 208 (or a variant thereof), an LCDR2 comprising the amino acid sequence set forth in SEQ ID NO: 209 (or a variant thereof), and an LCDR3 comprising the amino acid sequence set forth in SEQ ID NO: 210 (or a variant thereof); (v) a HCVR that comprises: an HCDR1 comprising the amino acid sequence set forth in SEQ ID NO: 213 (or a variant thereof), an HCDR2 comprising the amino acid sequence set forth in SEQ ID NO: 214 (or a variant thereof), and an HCDR3 comprising the amino acid sequence set forth in SEQ ID NO: 215 (or a variant thereof); and a LCVR that comprises: an LCDR1 comprising the amino acid sequence set forth in SEQ ID NO: 218 (or a variant thereof), an LCDR2 comprising the amino acid sequence set forth in SEQ ID NO: 219 (or a variant thereof), and an LCDR3 comprising the amino acid sequence set forth in SEQ ID NO: 220 (or a variant thereof); (w) a HCVR that comprises: an HCDR1 comprising the amino acid sequence set forth in SEQ ID NO: 223 (or a variant thereof), an HCDR2 comprising the amino acid sequence set forth in SEQ ID NO: 224 (or a variant thereof), and an HCDR3 comprising the amino acid sequence set forth in SEQ ID NO: 225 (or a variant thereof); and a LCVR that comprises: an LCDR1 comprising the amino acid sequence set forth in SEQ ID NO: 228 (or a variant thereof), an LCDR2 comprising the amino acid sequence set forth in SEQ ID NO: 229 (or a variant thereof), and an LCDR3 comprising the amino acid sequence set forth in SEQ ID NO: 230 (or a variant thereof); (x) a HCVR that comprises: an HCDR1 comprising the amino acid sequence set forth in SEQ ID NO: 233 (or a variant thereof), an HCDR2 comprising the amino acid sequence set forth in SEQ ID NO: 234 (or a variant thereof), and an HCDR3 comprising the amino acid sequence set forth in SEQ ID NO: 235 (or a variant thereof); and a LCVR that comprises: an LCDR1 comprising the amino acid sequence set forth in SEQ ID NO: 238 (or a variant thereof), an LCDR2 comprising the amino acid sequence set forth in SEQ ID NO: 239 (or a variant thereof), and an LCDR3 comprising the amino acid sequence set forth in SEQ ID NO: 240 (or a variant thereof); (y) a HCVR that comprises: an HCDR1 comprising the amino acid sequence set forth in SEQ ID NO: 243 (or a variant thereof), an HCDR2 comprising the amino acid sequence set forth in SEQ ID NO: 244 (or a variant thereof), and an HCDR3 comprising the amino acid sequence set forth in SEQ ID NO: 245 (or a variant thereof); and a LCVR that comprises: an LCDR1 comprising the amino acid sequence set forth in SEQ ID NO: 248 (or a variant thereof), an LCDR2 comprising the amino acid sequence set forth in SEQ ID NO: 249 (or a variant thereof), and an LCDR3 comprising the amino acid sequence set forth in SEQ ID NO: 250 (or a variant thereof); (z) a HCVR that comprises: an HCDR1 comprising the amino acid sequence set forth in SEQ ID NO: 253 (or a variant thereof), an HCDR2 comprising the amino acid sequence set forth in SEQ ID NO: 254 (or a variant thereof), and an HCDR3 comprising the amino acid sequence set forth in SEQ ID NO: 255 (or a variant thereof); and a LCVR that comprises: an LCDR1 comprising the amino acid sequence set forth in SEQ ID NO: 258 (or a variant thereof), an LCDR2 comprising the amino acid sequence set forth in SEQ ID NO: 259 (or a variant thereof), and an LCDR3 comprising the amino acid sequence set forth in SEQ ID NO: 260 (or a variant thereof); (aa) a HCVR that comprises: an HCDR1 comprising the amino acid sequence set forth in SEQ ID NO: 263 (or a variant thereof), an HCDR2 comprising the amino acid sequence set forth in SEQ ID NO: 264 (or a variant thereof), and an HCDR3 comprising the amino acid sequence set forth in SEQ ID NO: 265 (or a variant thereof); and a LCVR that comprises: an LCDR1 comprising the amino acid sequence set forth in SEQ ID NO: 268 (or a variant thereof), an LCDR2 comprising the amino acid sequence set forth in SEQ ID NO: 269 (or a variant thereof), and an LCDR3 comprising the amino acid sequence set forth in SEQ ID NO: 270 (or a variant thereof); (ab) a HCVR that comprises: an HCDR1 comprising the amino acid sequence set forth in SEQ ID NO: 273 (or a variant thereof), an HCDR2 comprising the amino acid sequence set forth in SEQ ID NO: 274 (or a variant thereof), and an HCDR3 comprising the amino acid sequence set forth in SEQ ID NO: 275 (or a variant thereof); and a LCVR that comprises: an LCDR1 comprising the amino acid sequence set forth in SEQ ID NO: 278 (or a variant thereof), an LCDR2 comprising the amino acid sequence set forth in SEQ ID NO: 279 (or a variant thereof), and an LCDR3 comprising the amino acid sequence set forth in SEQ ID NO: 280 (or a variant thereof); (ac) a HCVR that comprises: an HCDR1 comprising the amino acid sequence set forth in SEQ ID NO: 283 (or a variant thereof), an HCDR2 comprising the amino acid sequence set forth in SEQ ID NO: 284 (or a variant thereof), and an HCDR3 comprising the amino acid sequence set forth in SEQ ID NO: 285 (or a variant thereof); and a LCVR that comprises: an LCDR1 comprising the amino acid sequence set forth in SEQ ID NO: 288 (or a variant thereof), an LCDR2 comprising the amino acid sequence set forth in SEQ ID NO: 289 (or a variant thereof), and an LCDR3 comprising the amino acid sequence set forth in SEQ ID NO: 290 (or a variant thereof); (ad) a HCVR that comprises: an HCDR1 comprising the amino acid sequence set forth in SEQ ID NO: 293 (or a variant thereof), an HCDR2 comprising the amino acid sequence set forth in SEQ ID NO: 294 (or a variant thereof), and an HCDR3 comprising the amino acid sequence set forth in SEQ ID NO: 295 (or a variant thereof); and a LCVR that comprises: an LCDR1 comprising the amino acid sequence set forth in SEQ ID NO: 298 (or a variant thereof), an LCDR2 comprising the amino acid sequence set forth in SEQ ID NO: 299 (or a variant thereof), and an LCDR3 comprising the amino acid sequence set forth in SEQ ID NO: 300 (or a variant thereof); (ae) a HCVR that comprises: an HCDR1 comprising the amino acid sequence set forth in SEQ ID NO: 303 (or a variant thereof), an HCDR2 comprising the amino acid sequence set forth in SEQ ID NO: 304 (or a variant thereof), and an HCDR3 comprising the amino acid sequence set forth in SEQ ID NO: 305 (or a variant thereof); and a LCVR that comprises: an LCDR1 comprising the amino acid sequence set forth in SEQ ID NO: 308 (or a variant thereof), an LCDR2 comprising the amino acid sequence set forth in SEQ ID NO: 309 (or a variant thereof), and an LCDR3 comprising the amino acid sequence set forth in SEQ ID NO: 310 (or a variant thereof); and/or (af) a HCVR that comprises: an HCDR1 comprising the amino acid sequence set forth in SEQ ID NO: 313 (or a variant thereof), an HCDR2 comprising the amino acid sequence set forth in SEQ ID NO: 314 (or a variant thereof), and an HCDR3 comprising the amino acid sequence set forth in SEQ ID NO: 315 (or a variant thereof); and a LCVR that comprises: an LCDR1 comprising the amino acid sequence set forth in SEQ ID NO: 318 (or a variant thereof), an LCDR2 comprising the amino acid sequence set forth in SEQ ID NO: 319 (or a variant thereof), and an LCDR3 comprising the amino acid sequence set forth in SEQ ID NO: 320 (or a variant thereof). In an embodiment, the fusion protein comprises: (w) a HCVR that comprises: an HCDR1 comprising the amino acid sequence set forth in SEQ ID NO: 223 (or a variant thereof), an HCDR2 comprising the amino acid sequence set forth in SEQ ID NO: 224 (or a variant thereof), and an HCDR3 comprising the amino acid sequence set forth in SEQ ID NO: 225 (or a variant thereof); and a LCVR that comprises: an LCDR1 comprising the amino acid sequence set forth in SEQ ID NO: 228 (or a variant thereof), an LCDR2 comprising the amino acid sequence set forth in SEQ ID NO: 229 (or a variant thereof), and an LCDR3 comprising the amino acid sequence set forth in SEQ ID NO: 230 (or a variant thereof); or (y) a HCVR that comprises: an HCDR1 comprising the amino acid sequence set forth in SEQ ID NO: 243 (or a variant thereof), an HCDR2 comprising the amino acid sequence set forth in SEQ ID NO: 244 (or a variant thereof), and an HCDR3 comprising the amino acid sequence set forth in SEQ ID NO: 245 (or a variant thereof); and a LCVR that comprises: an LCDR1 comprising the amino acid sequence set forth in SEQ ID NO: 248 (or a variant thereof), an LCDR2 comprising the amino acid sequence set forth in SEQ ID NO: 249 (or a variant thereof), and an LCDR3 comprising the amino acid sequence set forth in SEQ ID NO: 250 (or a variant thereof. In an embodiment, the fusion protein comprises: (w) a HCVR that comprises: an HCDR1 comprising the amino acid sequence set forth in SEQ ID NO: 223 (or a variant thereof), an HCDR2 comprising the amino acid sequence set forth in SEQ ID NO: 224 (or a variant thereof), and an HCDR3 comprising the amino acid sequence set forth in SEQ ID NO: 225 (or a variant thereof); and a LCVR that comprises: an LCDR1 comprising the amino acid sequence set forth in SEQ ID NO: 228 (or a variant thereof), an LCDR2 comprising the amino acid sequence set forth in SEQ ID NO: 229 (or a variant thereof), and an LCDR3 comprising the amino acid sequence set forth in SEQ ID NO: 230 (or a variant thereof). In an embodiment, the fusion protein comprises: (y) a HCVR that comprises: an HCDR1 comprising the amino acid sequence set forth in SEQ ID NO: 243 (or a variant thereof), an HCDR2 comprising the amino acid sequence set forth in SEQ ID NO: 244 (or a variant thereof), and an HCDR3 comprising the amino acid sequence set forth in SEQ ID NO: 245 (or a variant thereof); and a LCVR that comprises: an LCDR1 comprising the amino acid sequence set forth in SEQ ID NO: 248 (or a variant thereof), an LCDR2 comprising the amino acid sequence set forth in SEQ ID NO: 249 (or a variant thereof), and an LCDR3 comprising the amino acid sequence set forth in SEQ ID NO: 250 (or a variant thereof. In an embodiment, the fusion protein comprises: (n) a HCVR that comprises: an HCDR1 comprising the amino acid sequence set forth in SEQ ID NO: 133 (or a variant thereof), an HCDR2 comprising the amino acid sequence set forth in SEQ ID NO: 134 (or a variant thereof), and an HCDR3 comprising the amino acid sequence set forth in SEQ ID NO: 135 (or a variant thereof); and a LCVR that comprises: an LCDR1 comprising the amino acid sequence set forth in SEQ ID NO: 138 (or a variant thereof), an LCDR2 comprising the amino acid sequence set forth in SEQ ID NO: 139 (or a variant thereof), and an LCDR3 comprising the amino acid sequence set forth in SEQ ID NO: 140 (or a variant thereof). In an embodiment, the fusion protein comprises: (r) a HCVR that comprises: an HCDR1 comprising the amino acid sequence set forth in SEQ ID NO: 173 (or a variant thereof), an HCDR2 comprising the amino acid sequence set forth in SEQ ID NO: 174 (or a variant thereof), and an HCDR3 comprising the amino acid sequence set forth in SEQ ID NO: 175 (or a variant thereof); and a LCVR that comprises: an LCDR1 comprising the amino acid sequence set forth in SEQ ID NO: 178 (or a variant thereof), an LCDR2 comprising the amino acid sequence set forth in SEQ ID NO: 179 (or a variant thereof), and an LCDR3 comprising the amino acid sequence set forth in SEQ ID NO: 180 (or a variant thereof). In an embodiment, the fusion protein comprises: (aa) a HCVR that comprises: an HCDR1 comprising the amino acid sequence set forth in SEQ ID NO: 263 (or a variant thereof), an HCDR2 comprising the amino acid sequence set forth in SEQ ID NO: 264 (or a variant thereof), and an HCDR3 comprising the amino acid sequence set forth in SEQ ID NO: 265 (or a variant thereof); and a LCVR that comprises: an LCDR1 comprising the amino acid sequence set forth in SEQ ID NO: 268 (or a variant thereof), an LCDR2 comprising the amino acid sequence set forth in SEQ ID NO: 269 (or a variant thereof), and an LCDR3 comprising the amino acid sequence set forth in SEQ ID NO: 270 (or a variant thereof). In an embodiment, the fusion protein comprises: (ab) a HCVR that comprises: an HCDR1 comprising the amino acid sequence set forth in SEQ ID NO: 273 (or a variant thereof), an HCDR2 comprising the amino acid sequence set forth in SEQ ID NO: 274 (or a variant thereof), and an HCDR3 comprising the amino acid sequence set forth in SEQ ID NO: 275 (or a variant thereof); and a LCVR that comprises: an LCDR1 comprising the amino acid sequence set forth in SEQ ID NO: 278 (or a variant thereof), an LCDR2 comprising the amino acid sequence set forth in SEQ ID NO: 279 (or a variant thereof), and an LCDR3 comprising the amino acid sequence set forth in SEQ ID NO: 280 (or a variant thereof).

In an embodiment, the fusion protein comprises: (i) a HCVR that comprises the amino acid sequence set forth in SEQ ID NO: 2 (or a variant thereof); and a LCVR that comprises the amino acid sequence set forth in SEQ ID NO: 7 (or a variant thereof); (ii) a HCVR that comprises the amino acid sequence set forth in SEQ ID NO: 12 (or a variant thereof); and a LCVR that comprises the amino acid sequence set forth in SEQ ID NO: 17 (or a variant thereof); (iii) a HCVR that comprises the amino acid sequence set forth in SEQ ID NO: 22 (or a variant thereof); and a LCVR that comprises the amino acid sequence set forth in SEQ ID NO: 27 (or a variant thereof); (iv) a HCVR that comprises the amino acid sequence set forth in SEQ ID NO: 32 (or a variant thereof); and a LCVR that comprises the amino acid sequence set forth in SEQ ID NO: 37 (or a variant thereof); (v) a HCVR that comprises the amino acid sequence set forth in SEQ ID NO: 42 (or a variant thereof); and a LCVR that comprises the amino acid sequence set forth in SEQ ID NO: 47 (or a variant thereof); (vi) a HCVR that comprises the amino acid sequence set forth in SEQ ID NO: 52 (or a variant thereof); and a LCVR that comprises the amino acid sequence set forth in SEQ ID NO: 57 (or a variant thereof); (vii) a HCVR that comprises the amino acid sequence set forth in SEQ ID NO: 62 (or a variant thereof); and a LCVR that comprises the amino acid sequence set forth in SEQ ID NO: 67 (or a variant thereof); (viii) a HCVR that comprises the amino acid sequence set forth in SEQ ID NO: 72 (or a variant thereof); and a LCVR that comprises the amino acid sequence set forth in SEQ ID NO: 77 (or a variant thereof); (ix) a HCVR that comprises the amino acid sequence set forth in SEQ ID NO: 82 (or a variant thereof); and a LCVR that comprises the amino acid sequence set forth in SEQ ID NO: 87 (or a variant thereof); (x) a HCVR that comprises the amino acid sequence set forth in SEQ ID NO: 92 (or a variant thereof); and a LCVR that comprises the amino acid sequence set forth in SEQ ID NO: 97 (or a variant thereof); (xi) a HCVR that comprises the amino acid sequence set forth in SEQ ID NO: 102 (or a variant thereof); and a LCVR that comprises the amino acid sequence set forth in SEQ ID NO: 107 (or a variant thereof); (xii) a HCVR that comprises the amino acid sequence set forth in SEQ ID NO: 112 (or a variant thereof); and a LCVR that comprises the amino acid sequence set forth in SEQ ID NO: 117 (or a variant thereof); (xiii) a HCVR that comprises the amino acid sequence set forth in SEQ ID NO: 122 (or a variant thereof); and a LCVR that comprises the amino acid sequence set forth in SEQ ID NO: 127 (or a variant thereof); (xiv) a HCVR that comprises the amino acid sequence set forth in SEQ ID NO: 132 (or a variant thereof); and a LCVR that comprises the amino acid sequence set forth in SEQ ID NO: 137 (or a variant thereof); (xv) a HCVR that comprises the amino acid sequence set forth in SEQ ID NO: 142 (or a variant thereof); and a LCVR that comprises the amino acid sequence set forth in SEQ ID NO: 147 (or a variant thereof); (xvi) a HCVR that comprises the amino acid sequence set forth in SEQ ID NO: 152 (or a variant thereof); and a LCVR that comprises the amino acid sequence set forth in SEQ ID NO: 157 (or a variant thereof); (xvii) a HCVR that comprises the amino acid sequence set forth in SEQ ID NO: 162 (or a variant thereof); and a LCVR that comprises the amino acid sequence set forth in SEQ ID NO: 167 (or a variant thereof); (xviii) a HCVR that comprises the amino acid sequence set forth in SEQ ID NO: 172 (or a variant thereof); and a LCVR that comprises the amino acid sequence set forth in SEQ ID NO: 177 (or a variant thereof); (xix) a HCVR that comprises the amino acid sequence set forth in SEQ ID NO: 182 (or a variant thereof); and a LCVR that comprises the amino acid sequence set forth in SEQ ID NO: 187 (or a variant thereof); (xx) a HCVR that comprises the amino acid sequence set forth in SEQ ID NO: 192 (or a variant thereof); and a LCVR that comprises the amino acid sequence set forth in SEQ ID NO: 197 (or a variant thereof); (xxi) a HCVR that comprises the amino acid sequence set forth in SEQ ID NO: 202 (or a variant thereof); and a LCVR that comprises the amino acid sequence set forth in SEQ ID NO: 207 (or a variant thereof); (xxii) a HCVR that comprises the amino acid sequence set forth in SEQ ID NO: 212 (or a variant thereof); and a LCVR that comprises the amino acid sequence set forth in SEQ ID NO: 217 (or a variant thereof); (xxiii) a HCVR that comprises the amino acid sequence set forth in SEQ ID NO: 222 (or a variant thereof); and a LCVR that comprises the amino acid sequence set forth in SEQ ID NO: 227 (or a variant thereof); (xxiv) a HCVR that comprises the amino acid sequence set forth in SEQ ID NO: 232 (or a variant thereof); and a LCVR that comprises the amino acid sequence set forth in SEQ ID NO: 237 (or a variant thereof); (xxv) a HCVR that comprises the amino acid sequence set forth in SEQ ID NO: 242 (or a variant thereof); and a LCVR that comprises the amino acid sequence set forth in SEQ ID NO: 247 (or a variant thereof); (xxvi) a HCVR that comprises the amino acid sequence set forth in SEQ ID NO: 252 (or a variant thereof); and a LCVR that comprises the amino acid sequence set forth in SEQ ID NO: 257 (or a variant thereof); (xxvii) a HCVR that comprises the amino acid sequence set forth in SEQ ID NO: 262 (or a variant thereof); and a LCVR that comprises the amino acid sequence set forth in SEQ ID NO: 267 (or a variant thereof); (xxviii) a HCVR that comprises the amino acid sequence set forth in SEQ ID NO: 272 (or a variant thereof); and a LCVR that comprises the amino acid sequence set forth in SEQ ID NO: 277 (or a variant thereof); (xxix) a HCVR that comprises the amino acid sequence set forth in SEQ ID NO: 282 (or a variant thereof); and a LCVR that comprises the amino acid sequence set forth in SEQ ID NO: 287 (or a variant thereof); (xxx) a HCVR that comprises the amino acid sequence set forth in SEQ ID NO: 292 (or a variant thereof); and a LCVR that comprises the amino acid sequence set forth in SEQ ID NO: 297 (or a variant thereof); (xxxi) a HCVR that comprises the amino acid sequence set forth in SEQ ID NO: 302 (or a variant thereof); and a LCVR that comprises the amino acid sequence set forth in SEQ ID NO: 307 or 488 (or a variant thereof); and/or (xxxii) a HCVR that comprises the amino acid sequence set forth in SEQ ID NO: 312 (or a variant thereof); and a LCVR that comprises the amino acid sequence set forth in SEQ ID NO: 317 (or a variant thereof). In an embodiment, the fusion protein comprises: (xxiii) a HCVR that comprises the amino acid sequence set forth in SEQ ID NO: 222 (or a variant thereof); and a LCVR that comprises the amino acid sequence set forth in SEQ ID NO: 227 (or a variant thereof); or (xxv) a HCVR that comprises the amino acid sequence set forth in SEQ ID NO: 242 (or a variant thereof); and a LCVR that comprises the amino acid sequence set forth in SEQ ID NO: 247 (or a variant thereof). In an embodiment, the fusion protein comprises: (xxiii i) a HCVR that comprises the amino acid sequence set forth in SEQ ID NO: 222 (or a variant thereof); and a LCVR that comprises the amino acid sequence set forth in SEQ ID NO: 227 (or a variant thereof). In an embodiment, the fusion protein comprises: (xxv) a HCVR that comprises the amino acid sequence set forth in SEQ ID NO: 242 (or a variant thereof); and a LCVR that comprises the amino acid sequence set forth in SEQ ID NO: 247 (or a variant thereof). In an embodiment, the fusion protein comprises: (xiv) a HCVR that comprises the amino acid sequence set forth in SEQ ID NO: 132 (or a variant thereof); and a LCVR that comprises the amino acid sequence set forth in SEQ ID NO: 137 (or a variant thereof). In an embodiment, the fusion protein comprises: (xviii) a HCVR that comprises the amino acid sequence set forth in SEQ ID NO: 172 (or a variant thereof); and a LCVR that comprises the amino acid sequence set forth in SEQ ID NO: 177 (or a variant thereof). In an embodiment, the fusion protein comprises: (xxvii) a HCVR that comprises the amino acid sequence set forth in SEQ ID NO: 262 (or a variant thereof); and a LCVR that comprises the amino acid sequence set forth in SEQ ID NO: 267 (or a variant thereof). In an embodiment, the fusion protein comprises: (xxviii) a HCVR that comprises the amino acid sequence set forth in SEQ ID NO: 272 (or a variant thereof); and a LCVR that comprises the amino acid sequence set forth in SEQ ID NO: 277 (or a variant thereof).

Provided herein is a fusion protein that is an scFv that comprises a heavy chain variable region (V_{H}) and a light chain variable region (V_{L}), and a payload such as an alpha-glucosidase polypeptide (GAA), wherein said V_{H}, V_{L} and payload, e.g., GAA, are arranged as follows: (i) V_{L}-V_{H}-Payload; (ii) V_{H}-V_{L}-Payload; (iii) V_{L}-[(GGGGS)₃ (SEQ ID NO: 538)]-V_{H}-[(GGGGS)₂ (SEQ ID NO: 537)]-Payload, or (iv) V_{H}-[(GGGGS)₃ (SEQ ID NO: 538)]-V_{L}-[(GGGGS)₂ (SEQ ID NO: 537)]-Payload. For example, in an embodiment, the fusion protein comprises (i) the amino acid sequence set forth in SEQ ID NO: 321 (or a mature polypeptide thereof), (ii) the amino acid sequence set forth in SEQ ID NO: 322 (or a mature polypeptide thereof), (iii) the amino acid sequence set forth in SEQ ID NO: 323 (or a mature polypeptide thereof), (iv) the amino acid sequence set forth in SEQ ID NO: 324 (or a mature polypeptide thereof), (v) amino acids 30-1168 of SEQ ID NO: 321, (vi) amino acids 30-1171 of SEQ ID NO: 322, (vii) amino acids 30-1164 of SEQ ID NO: 323, or (viii) amino acids 30-1166 of SEQ ID NO: 324. In an embodiment, the scFv comprises the amino acid sequence set forth in SEQ ID NO: 443 (or a variant thereof) or comprises the amino acid sequence set forth in SEQ ID NO: 440 (or a variant thereof). In an embodiment, the scFv comprises the amino acid sequence set forth in SEQ ID NO: 443 (or a variant thereof). In an embodiment, the scFv comprises the amino acid sequence set forth in SEQ ID NO: 440 (or a variant thereof). In an embodiment, the scFv comprises the amino acid sequence set forth in SEQ ID NO: 429 (or a variant thereof). In an embodiment, the scFv comprises the amino acid sequence set forth in SEQ ID NO: 433 (or a variant thereof). In an embodiment, the scFv comprises the amino acid sequence set forth in SEQ ID NO: 442 (or a variant thereof). In an embodiment, the scFv comprises the amino acid sequence set forth in SEQ ID NO: 438 (or a variant thereof). In an embodiment, the fusion protein comprises the amino acid sequence set forth in SEQ ID NO: 408 (or a variant thereof) or comprises the amino acid sequence set forth in SEQ ID NO: 405 (or a variant thereof). In an embodiment, the fusion protein comprises the amino acid sequence set forth in SEQ ID NO: 408 (or a variant thereof). In an embodiment, the fusion protein comprises the amino acid sequence set forth in SEQ ID NO: 405 (or a variant thereof). In an embodiment, the fusion protein (e.g., a Fab of said fusion protein) comprises the amino acid sequence set forth in a member selected from the group consisting of SEQ ID NOs: 392-423, e.g., which is fused to a payload such as a GAA polypeptide. In an embodiment, an anti-TfR:GAA fusion protein single chain fragment variable (scFv), antibody or an antigen-binding fragment thereof, which is not fused to a GAA polypeptide, does not block more than 50% of binding of a human transferrin receptor C-terminal fragment to human holo-transferrin that occurs in the absence of such single chain fragment variable (scFv), antibody or an antigen-binding fragment; for example, wherein said blocking is as measured in an Enzyme Linked Immunosorbent Assay (ELISA) plate assay wherein binding of human transferrin receptor C-terminal fragment that is fused to a His6-myc-myc tag is pre-bound to said scFv, antibody or antigen-binding fragment and then contacted with holo-transferrin which is immobilized to the surface of the plate by binding of an anti-holo-transferrin antibody that is bound to the plate, e.g., wherein binding of the holotransferrin and human transferrin receptor C in the absence of the scFv, antibody or antigen-binding fragment is measured at a concentration of about 300 pM human transferrin receptor C-terminal fragment.

Also provided is a composition that includes an anti-hTfR:GAA fusion protein provided herein, e.g., pharmaceutical composition comprising a fusion protein as disclosed herein and a pharmaceutically acceptable carrier. Kits including a fusion protein as disclosed herein are also provided. Such a composition or kit further including a further therapeutic agent (e.g., alglucosidase alfa, rituximab, methotrexate, Intravenous immunoglobulin (IVIG), avalglucosidase alfa, levalbuterol, an antibiotic, cortisone, prednisone, a bisphosphonate, and palivizumab, a Beta2-adrenergic agonist, a steroid, a bisphosphonate, an infectious disease treatment, a vaccine, and/or a Pneumococcal vaccine) is also provided.

Complexes comprising an anti-hTfR:GAA fusion protein as disclosed herein bound to a human transferrin receptor polypeptide or antigenic fragment thereof are also provided.

Also provided is an isolated polynucleotide encoding an anti-hTfR:GAA fusion protein disclosed herein, e.g., that comprises the nucleotide sequence set forth in SEQ ID NO: 1; 6; 11; 16; 21; 26; 31; 36; 41; 46; 51; 56; 61; 66; 71; 76; 81; 86; 91; 96; 101; 106; 111; 116; 121; 126; 131; 136; 141; 146; 151; 156; 161; 166; 171; 176; 181; 186; 191; 196; 201; 206; 211; 216; 221; 226; 231; 236; 241; 246; 251; 256; 261; 266; 271; 276; 281; 286; 291; 296; 301; 306; 311; and/or 316. For example, polynucleotides comprising any one or more of the following are provided: (1) the nucleotide sequence set forth in SEQ ID NO: 1 and SEQ ID NO: 6; (2) the nucleotide sequence set forth in SEQ ID NO: 11 and SEQ ID NO: 16; (3) the nucleotide sequence set forth in SEQ ID NO: 21 and SEQ ID NO: 26; (4) the nucleotide sequence set forth in SEQ ID NO: 31 and SEQ ID NO: 36; (5) the nucleotide sequence set forth in SEQ ID NO: 41 and SEQ ID NO: 46; (6) the nucleotide sequence set forth in SEQ ID NO: 51 and SEQ ID NO: 56; (7) the nucleotide sequence set forth in SEQ ID NO: 61 and SEQ ID NO: 66; (8) the nucleotide sequence set forth in SEQ ID NO: 71 and SEQ ID NO: 76; (9) the nucleotide sequence set forth in SEQ ID NO: 81 and SEQ ID NO: 86; (10) the nucleotide sequence set forth in SEQ ID NO: 91 and SEQ ID NO: 96; (11) the nucleotide sequence set forth in SEQ ID NO: 101 and SEQ ID NO: 106; (12) the nucleotide sequence set forth in SEQ ID NO: 111 and SEQ ID NO: 116; (13) the nucleotide sequence set forth in SEQ ID NO: 121 and SEQ ID NO: 126; (14) the nucleotide sequence set forth in SEQ ID NO: 131 and SEQ ID NO: 136; (15) the nucleotide sequence set forth in SEQ ID NO: 141 and SEQ ID NO: 146; (16) the nucleotide sequence set forth in SEQ ID NO: 151 and SEQ ID NO: 156; (17) the nucleotide sequence set forth in SEQ ID NO: 161 and SEQ ID NO: 166; (18) the nucleotide sequence set forth in SEQ ID NO: 171 and SEQ ID NO: 176; (19) the nucleotide sequence set forth in SEQ ID NO: 181 and SEQ ID NO: 186; (20) the nucleotide sequence set forth in SEQ ID NO: 191 and SEQ ID NO: 196; (21) the nucleotide sequence set forth in SEQ ID NO: 201 and SEQ ID NO: 206; (22) the nucleotide sequence set forth in SEQ ID NO: 211 and SEQ ID NO: 216; (23) the nucleotide sequence set forth in SEQ ID NO: 221 and SEQ ID NO: 226; (24) the nucleotide sequence set forth in SEQ ID NO: 231 and SEQ ID NO: 236; (25) the nucleotide sequence set forth in SEQ ID NO: 241 and SEQ ID NO: 246; (26) the nucleotide sequence set forth in SEQ ID NO: 251 and SEQ ID NO: 256; (27) the nucleotide sequence set forth in SEQ ID NO: 261 and SEQ ID NO: 266; (28) the nucleotide sequence set forth in SEQ ID NO: 271 and SEQ ID NO: 276; (29) the nucleotide sequence set forth in SEQ ID NO: 281 and SEQ ID NO: 286; (30) the nucleotide sequence set forth in SEQ ID NO: 291 and SEQ ID NO: 296; (31) the nucleotide sequence set forth in SEQ ID NO: 301 and SEQ ID NO: 306; and/or (32) the nucleotide sequence set forth in SEQ ID NO: 311 and SEQ ID NO: 316. A vector, e.g., an expression vector, comprising a polynucleotide encoding a fusion protein disclosed herein is provided. Also provided is a host cell (Chinese hamster ovary (CHO) cell) comprising the fusion protein, polynucleotide and/or vector.

Also provided is a method for making an anti-hTfR:GAA fusion protein as disclosed herein comprises the steps of culturing a host cell (e.g., CHO cell) comprising a polynucleotide that encodes the fusion protein in a culture medium under conditions favorable to expression of the fusion protein, e.g., (a) introducing said polynucleotide into a host cell; (b) culturing the host cell under conditions favorable to expression of the fusion protein; and (c) optionally, isolating the fusion protein from the culture medium and/or host cell. Such fusion proteins which are a product of such a method are provided.

Also provided herein is a method for administering (e.g., parenterally, e.g., intravenously) an anti-hTfR:GAA fusion protein as disclosed herein, optionally in association with a further therapeutic agent, to a subject (e.g., having a GSD and/or a GAA genotype selected from the group consisting of: ASP91ASN; MET318THR; GLU521LYS; GLY643ARG; ARG725TRP; IVS1AS, T-G, −13; LYS903DEL; LEU299ARG; SER529VAL; ASP645GLU; GLU689LYS; EX18DEL; PRO545LEU; 1-BP DEL, 525T; ARG854TER; ALA237VAL; GLY293ARG; and IVS6AS, G-C, −1) comprising introducing the protein into the body of the subject. Also provided herein is a method for treating or preventing a glycogen storage disease (e.g., Pompe disease, for example, classic infantile-onset form Pompe disease; non-classic infantile form Pompe disease; or late onset form Pompe disease), in a subject (e.g., having a GAA genotype selected from the group consisting of: ASP91ASN; MET318THR; GLU521LYS; GLY643ARG; ARG725TRP; IVS1AS, T-G, −13; LYS903DEL; LEU299ARG; SER529VAL; ASP645GLU; GLU689LYS; EX18DEL; PRO545LEU; 1-BP DEL, 525T; ARG854TER; ALA237VAL; GLY293ARG; and IVS6AS, G-C, −1) in need thereof comprising administering, to the subject, an effective amount of the fusion protein as disclosed herein, optionally in association with a further therapeutic agent. In an embodiment, the subject is 1 year of age or less and experiences a symptom selected from: Trouble eating and not gaining weight; Poor head and neck control; Rolling over and sitting up later than expected; Breathing problems; Lung infection; Enlarged and thickening heart; Heart defect; Enlarged liver; and Enlarged tongue. In an embodiment, the subject is an adult and experiences a symptom selected from: Weakness in the legs, trunk, and/or arms; Shortness of breath; Lung infection; Trouble breathing while sleeping; Spine curvature; Enlarged liver; Enlarged tongue; and Stiff joints.

Also provided herein is a method for delivering a payload (e.g., one or more antibodies or antigen-binding fragments thereof, proteins, enzymes or viral vectors containing one or more polynucleotides or oligonucleotides), e.g., GAA, to a tissue in the body of a subject (e.g., cartilage, brain, cerebral cortex; cerebellum; hippocampus; caudate; parathyroid gland; adrenal gland; bronchus; lung; oral mucosa; esophagus; stomach; duodenum; small intestine; colon; rectum; liver; gallbladder; pancreas; kidney; urinary bladder; testis; epididymis; prostate; vagina; ovary; fallopian tube; endometrium; cervix; placenta; breast; muscle, heart muscle; skeletal muscle, smooth muscle, muscle endothelial vasculature; soft tissue; skin; appendix; lymph node; tonsil; and/or bone marrow) including administering an antigen-binding protein that binds specifically to transferrin receptor or an antigenic-fragment thereof or variant thereof to the subject fused to the payload. For example, the method can include the steps of piercing the body of the subject with a needle of a syringe and injecting antigen-binding protein that binds specifically to transferrin receptor or an antigenic-fragment thereof or variant thereof to the subject fused to the payload into the body of the subject.

### BRIEF DESCRIPTION OF THE FIGURES

FIG. 1. Amino acid sequences of various anti-human transferrin receptor scFv molecules in Vₖ-3×G₄S(SEQ ID NO: 538)-V_{H} format which are provided herein.

FIGS. 2A-2C. Anti-human TFRC scFv antibody clones deliver GAA to the cerebrum of Tfrc^{hum} mice. Anti-human TfR:GAA molecules 69261, 69329, 12839, 12841, 12843 and 12845 (FIG. 2A) 69348, 12795, 12799, 12801, 12850 and 12798 (FIG. 2B); and 12802, 69340, 12847, 12848, 69307 and 69323 (FIG. 2C) were tested. Each lane=1 mouse. Delivery by HDD. Quantified in Table 4-1.

FIG. 3. A subset of anti-hTFRC antibodies (12798, 12850, 69323, 12841, 12843, 12845, 12847, 12848, 12799, 69307 and 12839) delivered mature GAA to the brain parenchyma in scfv:GAA format (delivery by HDD). Lane E corresponds to endothelium and Lane P corresponds to parenchyma. Ratio of affinity for mfTfR:human TfR are indicated below the image (mf refers to Macaca fascicularis monkey). Quantified in Tables 4-2 and 4-3.

FIG. 4. Anti-hTFRC antibodies (12799, 12843, 12847 and 12839) delivered mature GAA to the brain parenchyma in scfv:GAA format (AAV8 episomal liver depot gene therapy). Lane E corresponds to endothelium and Lane P corresponds to parenchyma. Quantified in Table 4-4.

FIG. 5. Episomal AAV8 liver depot anti-hTFRC scfv:GAA antibodies delivered GAA protein to CNS (cerebellum, cerebrum, spinal cord), heart, and muscle (quadricep) in Gaa^{-/-}/Tfrc^{hum} mice. Quantified in Table 4-5.

FIG. 6. Episomal AAV8 liver depot anti-hTFRC scfv:GAA antibodies (12839, 12843 and 12847) rescued glycogen storage in central nervous system (CNS) (cerebellum, cerebrum, spinal cord), heart, and muscle (quadricep) in Gaa^{-/-}/Tfrc^{hum} mice. Quantified in Table 4-6.

FIGS. 7A-7D. Episomal AAV8 liver depot anti-hTFRC scfv:GAA antibodies (12847, 12843 and 12799) rescued glycogen storage in brain (brain thalamus (FIG. 7A), brain cerebral cortex (FIG. 7B), brain hippocampus CA1 (FIG. 7C)) and muscle (quadricep (FIG. 7D)) in Gaa^{-/-}/Tfrc^{hum} mice.

FIG. 8. Albumin insertion of anti-hTFRC 12847scfv:GAA delivers mature GAA protein to CNS and muscle of Pompe model mice.

FIG. 9. Albumin insertion of anti-hTFRC 12847scfv:GAA rescues glycogen storage in CNS and muscle of Pompe model mice. One Way ANOVA (*p<0.01; **p<0.001; ***p<0.0001).

FIG. 10. GAA activity in serum following Cas9-mediated insertion of AAV-delivered anti-TfR1:GAA or anti-CD63:GAA into the cynomolgus monkey albumin locus. Vehicle-only was used as a negative control. One unit of GAA activity is defined as the amount of enzyme that generates 1.0 μmol of 4-MU per min at pH 4.5 at 37° C. Error bars are SEM. N=1 for vehicle; N=2-4 for all others.

FIG. 11. Albumin insertion of anti-hTFRC 12847scfv:GAA delivers mature GAA protein to CNS and muscle of cynomolgus monkeys. For the bar graphs, mature GAA was quantified by western blot of tissue lysates, and error bars are SD.

FIG. 12 shows the interaction of Mammarenavirus machupoense GP1 protein (PDB 3KAS), human ferritin (PDB 6GSR), Plasmodium vivax Sal-1 PvRBP2b protein (PDB 6D04), human HFE protein (PDB 1DE4), and human transferrin (PDB 1 SUV) molecules superimposed on two TfR molecules in a symmetrical unit. For Mammarenavirus machupoense GP1 protein and human ferritin, only one copy in the symmetrical unit is shown to reduce complexity of the figure for clear view.

FIG. 13 depicts Hydrogen-Deuterium Exchange Mass Spectrometry (HDX) protections for the antibodies tested in HDX-MS experiments can be assigned to 5 regions in TfR (PDB 1 SUV).

FIG. 14 illustrates TfR regions protected by REGN17513, a representation of antibodies that cause HDX protections in TfR apical domain that overlap with Mammarenavirus machupoense GP1 protein, human ferritin, and Plasmodium vivax PvRBP2b protein binding sites.

FIG. 15 illustrates TfR regions protected by REGN17510, a representation of antibodies with HDX protections in TfR apical domain that are not shared by other TfR binding partners shown in FIG. 15.

FIG. 16 illustrates TfR regions protected by REGN17515, a representation of antibodies with HDX protections in TfR apical domain that share binding sites with human ferritin and Plasmodium vivax Sal-1 PvRBP2b protein.

FIG. 17 illustrates TfR regions protected by REGN17514, a representation of antibodies with HDX protections in TfR protease-like domain and share binding sites with Plasmodium vivax Sal-1 PvRBP2b protein.

FIG. 18 illustrates TfR regions protected by REGN17508, a representation of antibodies with HDX protections in TfR protease-like domain. This region is not utilized by other TfR interacting molecules shown in FIG. 18.

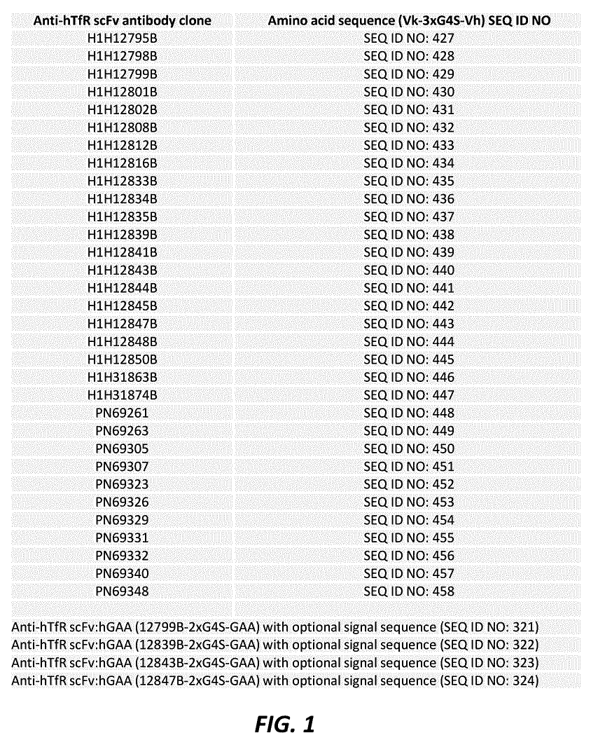

### DETAILED DESCRIPTION

### I. Overview

Provided herein are anti-transferrin receptor antigen-binding proteins. Also provided are anti-transferrin receptor antigen-binding proteins that are fused to a payload. Such fusions are useful, for example, for delivery of the payload to various tissues in the body, including the brain. For example, anti-TfR:GAA fusion proteins exhibiting high affinity to the transferrin receptor and superior blood-brain barrier crossing are provided. Surprisingly, fusions exhibiting high binding affinity to TfR crossed the BBB more efficiently than that of low affinity binders. We found that high affinity antibodies impart the best delivery to the CNS and muscle in the anti-hTFRscfv:payload (e.g., GAA) format. This is in contrast to previous findings with mono- and bivalent anti-TFR antibodies, where low affinity antibodies crossed the BBB more effectively. The fusions disclosed herein have an ability to efficiently deliver GAA to the brain and, thus, are an effective treatment of glycogen storage diseases such as Pompe Disease.

There may be employed herein conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994).

A polynucleotide includes DNA and RNA. Provided herein is any polynucleotide disclosed herein which is operably linked to a promoter or other expression control sequence.

A symptom is a manifestation of disease apparent to the patient himself, while a sign is a manifestation of disease that the physician perceives. Reduction, fully or in part, of a sign or symptom may be referred to as alleviation of the sign or symptom.

An oligonucleotide is a polynucleotide of up to about 30 nucleotides in length, e.g., about 20, 21, 22, 23, 24, 25, 26, 27, 28, 29 or 30 nucleotides.

Transferrin receptor 1 (TfR) is a membrane receptor involved in the control of iron supply to the cell through the binding of transferrin, the major iron-carrier protein. Transferrin receptor 1 is expressed from the TFRC gene. Transferrin receptor 1 may be referred to, herein, at TFRC. This receptor plays a key role in the control of cell proliferation because iron is essential for sustaining ribonucleotide reductase activity, and is the only enzyme that catalyzes the conversion of ribonucleotides to deoxyribonucleotides. Preferably, the TfR is human TfR (hTfR). See e.g., Accession numbers NP_001121620.1; BAD92491.1; and NP_001300894.1; and e!Ensembl entry: ENSG00000072274. The human transferrin receptor 1 is expressed in several tissues, including but not limited to: cerebral cortex; cerebellum; hippocampus; caudate; parathyroid gland; adrenal gland; bronchus; lung; oral mucosa; esophagus; stomach; duodenum; small intestine; colon; rectum; liver; gallbladder; pancreas; kidney; urinary bladder; testis; epididymis; prostate; vagina; ovary; fallopian tube; endometrium; cervix; placenta; breast; heart muscle; smooth muscle; soft tissue; skin; appendix; lymph node; tonsil; and bone marrow. See also tissues and cell types of Table B herein. A related transferrin receptor is transferrin receptor 2 (TfR2). Human transferrin receptor 2 bears about 45% sequence identity to human transferrin receptor 1. Trinder & Baker, Transferrin receptor 2: a new molecule in iron metabolism. Int J Biochem Cell Biol. 2003 March; 35(3):292-6. Unless otherwise stated, transferrin receptor as used herein generally refers to transferrin receptor 1 (e.g., human transferrin receptor 1) (CD71).

Human Transferrin (Tf) is a single chain, 80 kDa member of the anion-binding superfamily of proteins. Transferrin is a 698 amino acid precursor that is divided into a 19 aa signal sequence plus a 679 aa mature segment that typically contains 19 intrachain disulfide bonds. The N- and C-terminal flanking regions (or domains) bind ferric iron through the interaction of an obligate anion (e.g., bicarbonate) and four amino acids (His, Asp, and two Tyr). Apotransferrin (or iron-free) will initially bind one atom of iron at the C-terminus, and this is followed by subsequent iron binding by the N-terminus to form holotransferrin (diferric Tf, Holo-Tf). Through its C-terminal iron-binding domain, holotransferrin will interact with the TfR on the surface of cells where it is internalized into acidified endosomes. Iron dissociates from the Tf molecule within these endosomes, and is transported into the cytosol as ferrous iron. In addition to TfR, transferrin is reported to bind to cubulin, IGFBP3, microbial iron-binding proteins and liver-specific TfR2.

The blood-brain barrier (BBB) is located within the microvasculature of the brain, and it regulates passage of molecules from the blood to the brain. Burkhart et al., Accessing targeted nanoparticles to the brain: the vascular route. Curr Med Chem. 2014; 21(36):4092-9. The transcellular passage through the brain capillary endothelial cells can take place via 1) cell entry by leukocytes; 2) carrier-mediated influx of e.g., glucose by glucose transporter 1 (GLUT-1), amino acids by e.g., the L-type amino acid transporter 1 (LAT-1) and small peptides by e.g., organic anion-transporting peptide-B (OATP-B); 3) paracellular passage of small hydrophobic molecules; 4) adsorption-mediated transcytosis of e.g., albumin and cationized molecules; 5) passive diffusion of lipid soluble, non-polar solutes, including CO₂ and O₂; and 5) receptor-mediated transcytosis of e.g., insulin by the insulin receptor and Tf by the TfR. Johnsen et al., Targeting the transferrin receptor for brain drug delivery, Prog Neurobiol. 2019 October; 181:101665.

### II. Anti-Human Transferrin Receptor Antigen-Binding Proteins and Fusions

Provided are antigen-binding proteins, such as antibodies, antigen-binding fragments thereof, such as Fabs and scFvs, that bind specifically to the transferrin receptor, preferably the human transferrin receptor 1 (anti-hTfR). For example, in an embodiment, the anti-hTfR is in the form of a fusion protein. The fusion protein includes the anti-hTfR antigen-binding protein fused to a particular payload (anti-hTfR:Payload). The anti-hTfRs disclosed herein efficiently cross the blood-brain barrier (BBB) and can, thereby, deliver the fused payload to the brain.

An antigen-binding protein that specifically binds to transferrin receptor and fusions thereof, for example, a tag such as His₆ and/or myc (e.g., human transferrin receptor (e.g., REGN2431) or monkey transferrin receptor (e.g., REGN2054)) binds at about 25° C., e.g., in a surface plasmon resonance assay, with a K_{D} of about 20 nM or a higher affinity. Such an antigen-binding protein may be referred to as “anti-TfR”. In some embodiments, the antigen-binding protein binds to human transferrin receptor with a K_{D} of about 0.41 nM or a stronger affinity. In some embodiments, the antigen-binding protein binds to human transferrin receptor with a K_{D} of about 3 nM or a stronger affinity. In some embodiments, the antigen-binding protein binds to human transferrin receptor with a K_{D} of about 0.45 nM to 3 nM. In some embodiments, a Fab having an HCVR and LCVR binds to human transferrin receptor with a K_{D} of about 0.65 nM or a stronger affinity. In some embodiments, a fusion protein disclosed herein binds to human transferrin receptor with a K_{D} of about 1×10⁻⁷ M or a stronger affinity.

In an embodiment, an anti-hTfR scFv:Payload or anti-TfR:Payload scFv fusion protein includes an scFv comprising the arrangement of variable regions as follows LCVR-HCVR or HCVR-LCVR, wherein the HCVR and LCVR are optionally connected by a linker and the scFv is connected, optionally by a linker, to a payload (e.g., GAA or variant thereof) (e.g., LCVR-(Gly₄Ser)₃ (SEQ ID NO: 538)-HCVR-(Gly₄Ser)₂ (SEQ ID NO: 537))-Payload (e.g., mature human GAA); or LCVR-(Gly₄Ser)₃ (SEQ ID NO: 538)-HCVR-(Gly₄Ser)₂ (SEQ ID NO: 537))-Payload (e.g., mature human GAA)) (Gly₄Ser=SEQ ID NO: 426).

An anti-hTfR:Payload optionally comprises a signal peptide, connected to the antigen-binding protein that binds specifically to transferrin receptor (TfR), preferably, human transferrin receptor (hTfR) which is fused (optionally by a linker) to a payload such as GAA or a variant thereof. In an embodiment, the signal peptide is the mROR signal sequence (e.g., mROR signal sequence-LCVR-(Gly₄Ser)₃ (SEQ ID NO: 538)-HCVR-(Gly₄Ser)₂ (SEQ ID NO: 537))-Payload (e.g., mature human GAA); or LCVR-(Gly₄Ser)₃ (SEQ ID NO: 538)-HCVR-(Gly₄Ser)₂ (SEQ ID NO: 537))-Payload (e.g., mature human GAA)) (Gly₄Ser=SEQ ID NO: 426).

The term “fused” or “tethered” with regard to fused polypeptides refers to polypeptides joined directly or indirectly (e.g., via a linker or other polypeptide).

In an embodiment, the assignment of amino acids to each framework or CDR domain in an immunoglobulin is in accordance with the definitions of Sequences of Proteins of Immunological Interest, Kabat et al.; National Institutes of Health, Bethesda, Md.; 5^{th} ed.; NIH Publ. No. 91-3242 (1991); Kabat (1978) Adv. Prot. Chem. 32:1-75; Kabat et al., (1977) J. Biol. Chem. 252:6609-6616; Chothia, et al., (1987) J Mol. Biol. 196:901-917 or Chothia, et al., (1989) Nature 342: 878-883. Thus, provided herein are antibodies and antigen-binding fragments including the CDRs of a V_{H} and the CDRs of a V_{L}, which V_{H} and V_{L} comprise amino acid sequences as set forth herein (see e.g., sequences of Table A, or variants thereof), wherein the CDRs are as defined according to Kabat and/or Chothia.

Provided herein are antibodies that bind specifically to the human transferrin receptor 1. The term “antibody”, as used herein, refers to immunoglobulin molecules comprising four polypeptide chains, two heavy chains (HCs) and two light chains (LCs), inter-connected by disulfide bonds. In an embodiment, each antibody heavy chain (HC) comprises a heavy chain variable region (“HCVR” or “V_{H}”) (e.g., comprising SEQ ID NO: 2; 462; 12; 463; 22; 464; 32; 42; 52; 467; 62; 492; 72; 470; 82; 92; 472; 102; 112; 473; 122; 132; 142; 475; 152; 162; 477; 172; 182; 478; 192; 480; 202; 481; 212; 222; 232; 242; 252; 482; 262; 272; 282; 292; 302; 483 and/or 312, or a variant thereof) and a heavy chain constant region (e.g., human IgG, human IgG1 or human IgG4); and each antibody light chain (LC) comprises a light chain variable region (“LCVR or “V_{L}”) (e.g., SEQ ID NO: 7; 17; 27; 37; 465; 47; 466; 57; 468; 67; 469; 77; 471; 87; 97; 107; 117; 474; 127; 137; 147; 476; 157; 167; 177; 187; 479; 197; 207; 217; 227; 237; 247; 257; 267; 277; 287; 297; 307; 488; 317 and/or 484, or a variant thereof) and a light chain constant region (e.g., human kappa or human lambda). In an embodiment, each antibody heavy chain (HC) comprises a heavy chain variable region (“HCVR” or “V_{H}”) (e.g., comprising SEQ ID NO: 222 or 242, or a variant thereof) and a heavy chain constant region (e.g., human IgG, human IgG1 or human IgG4); and each antibody light chain (LC) comprises a light chain variable region (“LCVR or “V_{L}”) (e.g., SEQ ID NO: 227 or 247, or a variant thereof) and a light chain constant region (e.g., human kappa or human lambda). In an embodiment, each antibody heavy chain (HC) comprises a heavy chain variable region (“HCVR” or “V_{H}”) (e.g., comprising SEQ ID NO: 222, or a variant thereof) and a heavy chain constant region (e.g., human IgG, human IgG1 or human IgG4); and each antibody light chain (LC) comprises a light chain variable region (“LCVR or “V_{L}”) (e.g., SEQ ID NO: 227, or a variant thereof) and a light chain constant region (e.g., human kappa or human lambda). In an embodiment, each antibody heavy chain (HC) comprises a heavy chain variable region (“HCVR” or “V_{H}”) (e.g., comprising SEQ ID NO: 242, or a variant thereof) and a heavy chain constant region (e.g., human IgG, human IgG1 or human IgG4); and each antibody light chain (LC) comprises a light chain variable region (“LCVR or “V_{L}”) (e.g., SEQ ID NO: 247, or a variant thereof) and a light chain constant region (e.g., human kappa or human lambda). In an embodiment, each antibody heavy chain (HC) comprises a heavy chain variable region (“HCVR” or “V_{H}”) (e.g., comprising SEQ ID NO: 132, or a variant thereof) and a heavy chain constant region (e.g., human IgG, human IgG1 or human IgG4); and each antibody light chain (LC) comprises a light chain variable region (“LCVR or “V_{L}”) (e.g., SEQ ID NO: 137, or a variant thereof) and a light chain constant region (e.g., human kappa or human lambda). In an embodiment, each antibody heavy chain (HC) comprises a heavy chain variable region (“HCVR” or “V_{H}”) (e.g., comprising SEQ ID NO: 172, or a variant thereof) and a heavy chain constant region (e.g., human IgG, human IgG1 or human IgG4); and each antibody light chain (LC) comprises a light chain variable region (“LCVR or “V_{L}”) (e.g., SEQ ID NO: 177, or a variant thereof) and a light chain constant region (e.g., human kappa or human lambda). In an embodiment, each antibody heavy chain (HC) comprises a heavy chain variable region (“HCVR” or “V_{H}”) (e.g., comprising SEQ ID NO: 262, or a variant thereof) and a heavy chain constant region (e.g., human IgG, human IgG1 or human IgG4); and each antibody light chain (LC) comprises a light chain variable region (“LCVR or “V_{L}”) (e.g., SEQ ID NO: 267, or a variant thereof) and a light chain constant region (e.g., human kappa or human lambda). In an embodiment, each antibody heavy chain (HC) comprises a heavy chain variable region (“HCVR” or “V_{H}”) (e.g., comprising SEQ ID NO: 272, or a variant thereof) and a heavy chain constant region (e.g., human IgG, human IgG1 or human IgG4); and each antibody light chain (LC) comprises a light chain variable region (“LCVR or “V_{L}”) (e.g., SEQ ID NO: 277, or a variant thereof) and a light chain constant region (e.g., human kappa or human lambda). The V_{H} and V_{L} regions can be further subdivided into regions of hypervariability, termed complementarity determining regions (CDR), interspersed with regions that are more conserved, termed framework regions (FR). Each V_{H} and V_{L} comprises three CDRs and four FRs. Anti-TfR antibodies disclosed herein can also be fused to a payload such as GAA or a variant thereof.

An anti-TfR antigen-binding protein provided herein may be an antigen-binding fragment of an antibody which may be tethered to a payload. The terms “antigen-binding portion” or “antigen-binding fragment” of an antibody, as used herein, refers to an immunoglobulin molecule that binds antigen but that does not include all of the sequences of a full antibody (preferably, the full antibody is an IgG). Non-limiting examples of antigen-binding fragments include: (i) Fab fragments; (ii) F(ab′)₂ fragments; (iii) Fd fragments; (iv) Fv fragments; (v) single-chain Fv (scFv) molecules; and (vi) dAb fragments; consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies and small modular immunopharmaceuticals (SMIPs), are also encompassed within the expression “antigen-binding fragment,” as used herein.

As mentioned, an anti-TfR antigen-binding protein provided herein may be an scFv which may be tethered to a payload. An scFv (single chain fragment variable) has variable regions of heavy (V_{H}) and light (V_{L}) domains (in either order), which, preferably, are joined together by a flexible linker (e.g., peptide linker). The length of the flexible linker used to link both of the V regions may be important for yielding the correct folding of the polypeptide chain. Previously, it has been estimated that the peptide linker must span 3.5 nm (35 A) between the carboxy terminus of the variable domain and the amino terminus of the other domain without affecting the ability of the domains to fold and form an intact antigen-binding site (Huston et al., Protein engineering of single-chain Fv analogs and fusion proteins. Methods in Enzymology. 1991; 203:46-88). In an embodiment, the linker comprises an amino acid sequence of such length to separate the variable domains by about 3.5 nm.

In some embodiments, an anti-TfR antigen-binding protein described herein comprises a monovalent or “one-armed” antibody. The monovalent or “one-armed” antibodies as used herein refer to immunoglobulin proteins comprising a single variable domain. For example, the one-armed antibody may comprise a single variable domain within a Fab wherein the Fab is linked to at least one Fc fragment. In certain embodiments, the one-armed antibody comprises: (i) a heavy chain comprising a heavy chain constant region and a heavy chain variable region, (ii) a light chain comprising a light chain constant region and a light chain variable region, and (iii) a polypeptide comprising a Fc fragment or a truncated heavy chain. In certain embodiments, the Fc fragment or a truncated heavy chain comprised in the separate polypeptide is a “dummy Fc,” which refers to an Fc fragment that is not linked to an antigen binding domain. The one-armed antibodies of the present disclosure may comprise any of the HCVR/LCVR pairs or CDR amino acid sequences as set forth in Table A herein. One-armed antibodies comprising a full-length heavy chain, a full-length light chain and an additional Fc domain polypeptide can be constructed using standard methodologies (see, e.g., WO2010151792, which is incorporated herein by reference in its entirety), wherein the heavy chain constant region differs from the Fc domain polypeptide by at least two amino acids (e.g., H95R and Y96F according to the IMGT exon numbering system; or H435R and Y436F according to the EU numbering system). Such modifications are useful in purification of the monovalent antibodies (see WO2010151792).

An antigen-binding fragment of an antibody will, in an embodiment, comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody described herein include: (i) V_{H}-CH1; (ii) V_{H}-CH2; (iii) V_{H}-CH3; (iv) V_{H}-CH1-CH2; (v) V_{H}-CH1-CH2-CH3; (vi) V_{H}-CH2-CH3; (vii) V_{H}-CL; (viii) V_{L}-CH1; (ix) V_{L}-CH2; (x) V_{L}-CH3; (xi) V_{L}-CH1-CH2; (xii) VL-CH1-CH2-CH3; (xiii) V_{L}-CH2-CH3; and (xiv) V_{L}-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody described herein may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)). The present disclosure includes an antigen-binding fragment of an antigen-binding protein such as an antibody set forth herein.

Antigen-binding proteins (e.g., antibodies and antigen-binding fragments) may be monospecific or multi-specific (e.g., bispecific). Multispecific antigen-binding proteins are discussed further herein. The present disclosure includes monospecific as well as multispecific (e.g., bispecific) antigen-binding fragments comprising one or more variable domains from an antigen-binding protein that is specifically set forth herein.

The term “specifically binds” or “binds specifically” refers to those antigen-binding proteins (e.g., antibodies or antigen-binding fragments thereof) having a binding affinity to an antigen, such as human TfR protein, mouse TfR protein or monkey TfR protein, expressed as K_{D}, of at least about 10⁻⁹ M (e.g., 0.01, 0.1, 0.2, 0.3, 0.4, 0.5, 0.6, 0.7, 0.8, 0.9 or 1.0 nM), as measured by real-time, label free bio-layer interferometry assay, for example, at 25° C. or 37° C., e.g., an Octet® HTX biosensor, or by surface plasmon resonance, e.g., BIACORE™, or by solution-affinity ELISA. The present disclosure includes antigen-binding proteins that specifically bind to TfR protein. “Anti-TfR” refers to an antigen-binding protein (or other molecule), for example an antibody or antigen-binding fragment thereof, that binds specifically to TfR.

“Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules (e.g., minor or insignificant amounts of impurity may remain) or to an absence of water, buffers, or salts or to components of a pharmaceutical formulation that includes the antigen-binding proteins (e.g., antibodies or antigen-binding fragments).

The present disclosure includes antigen-binding proteins, e.g., antibodies or antigen-binding fragments, that bind to the same epitope as an antigen-binding protein described herein. In some embodiments, provided is an antigen-binding protein that binds specifically to transferrin receptor or an antigenic-fragment thereof or variant thereof which binds to one or more epitopes of hTfR selected from: (a) an epitope comprising the sequence LLNE (SEQ ID NO: 525) and/or an epitope comprising the sequence TYKEL (SEQ ID NO: 507); (b) an epitope comprising the sequence DSTDFTGT (SEQ ID NO: 526) and/or an epitope comprising the sequence VKHPVTGQF (SEQ ID NO: 527) and/or an epitope comprising the sequence IERIPEL (SEQ ID NO: 528); (c) an epitope comprising the sequence LNENSYVPREAGSQKDEN (SEQ ID NO: 529); (d) an epitope comprising the sequence FEDL (SEQ ID NO: 519); (e) an epitope comprising the sequence IVDKNGRL (SEQ ID NO: 530); (f) an epitope comprising the sequence IVDKNGRLVY (SEQ ID NO: 531); (g) an epitope comprising the sequence DQTKF (SEQ ID NO: 532); (h) an epitope comprising the sequence LVENPGGY (SEQ ID NO: 533) and/or an epitope comprising the sequence PIVNAELSF (SEQ ID NO: 534) and/or an epitope comprising the sequence PYLGTTMDT (SEQ ID NO: 535); (i) an epitope comprising the sequence LLNENSYVPREAGSQKDENLAL (SEQ ID NO: 505) and/or an epitope comprising the sequence IYMDQTKFPIVNAEL (SEQ ID NO: 506) and/or an epitope comprising the sequence TYKEL (SEQ ID NO: 507); (j) an epitope comprising the sequence KRKLSEKLDSTDFTGTIKL (SEQ ID NO: 508) and/or an epitope comprising the sequence YTLIEKTMQNVKHPVTGQFL (SEQ ID NO: 509) and/or an epitope comprising the sequence LIERIPELNKVARAAAE (SEQ ID NO: 510); (k) an epitope comprising the sequence LNENSYVPREAGSQKDENL (SEQ ID NO: 511); (1) an epitope comprising the sequence GTKKDFEDL (SEQ ID NO: 512); (m) an epitope comprising the sequence SVIIVDKNGRLVYLVENPGGYVAYSK (SEQ ID NO: 513); (n) an epitope comprising the sequence LLNENSYVPREAGSQKDEN (SEQ ID NO: 514) and/or an epitope comprising the sequence DQTKFPIVNAEL (SEQ ID NO: 515) and/or an epitope comprising the sequence TYKELIERIPELNK (SEQ ID NO: 516); (o) an epitope comprising the sequence LLNENSYVPREAGSQKDEN (SEQ ID NO: 514) and/or an epitope comprising the sequence TYKELIERIPELNK (SEQ ID NO: 516); (p) an epitope comprising the sequence SVIIVDKNGRLVYLVENPGGYVAY (SEQ ID NO: 517); (q) an epitope comprising the sequence IYMDQTKFPIVNAEL (SEQ ID NO: 506) and/or an epitope comprising the sequence FGNMEGDCPSDWKTDSTCRM (SEQ ID NO: 518); (r) an epitope comprising the sequence LLNENSYVPREAGSQKDENLAL (SEQ ID NO: 505) and/or an epitope comprising the sequence LVENPGGYVAYSKAATVTGKL (SEQ ID NO: 520) and/or an epitope comprising the sequence IYMDQTKFPIVNAELSF (SEQ ID NO: 521) and/or an epitope comprising the sequence ISRAAAEKL (SEQ ID NO: 522) and/or an epitope comprising the sequence VTSESKNVKLTVSNVLKE (SEQ ID NO: 523) and/or an epitope comprising the sequence FCEDTDYPYLGTTMDT (SEQ ID NO: 524); (s) an epitope comprised within or overlapping with the sequence LLNENSYVPREAGSQKDENLAL (SEQ ID NO: 505) and/or an epitope comprised within or overlapping with the sequence IYMDQTKFPIVNAEL (SEQ ID NO: 506) and/or an epitope comprised within or overlapping with the sequence TYKEL (SEQ ID NO: 507); (t) an epitope comprised within or overlapping with the sequence KRKLSEKLDSTDFTGTIKL (SEQ ID NO: 508) and/or an epitope comprised within or overlapping with the sequence YTLIEKTMQNVKHPVTGQFL (SEQ ID NO: 509) and/or an epitope comprised within or overlapping with the sequence LIERIPELNKVARAAAE (SEQ ID NO: 510); (u) an epitope comprised within or overlapping with the sequence LNENSYVPREAGSQKDENL (SEQ ID NO: 511); (v) an epitope comprised within or overlapping with the sequence GTKKDFEDL (SEQ ID NO: 512); (w) an epitope comprised within or overlapping with the sequence SVIIVDKNGRLVYLVENPGGYVAYSK (SEQ ID NO: 513); (x) an epitope comprised within or overlapping with the sequence LLNENSYVPREAGSQKDEN (SEQ ID NO: 514) and/or an epitope comprised within or overlapping with the sequence DQTKFPIVNAEL (SEQ ID NO: 515) and/or an epitope comprised within or overlapping with the sequence TYKELIERIPELNK (SEQ ID NO: 516); (y) an epitope comprised within or overlapping with the sequence LLNENSYVPREAGSQKDEN (SEQ ID NO: 514) and/or an epitope comprised within or overlapping with the sequence TYKELIERIPELNK (SEQ ID NO: 516); (z) an epitope comprised within or overlapping with the sequence SVIIVDKNGRLVYLVENPGGYVAY (SEQ ID NO: 517); (aa) an epitope comprised within or overlapping with the sequence IYMDQTKFPIVNAEL (SEQ ID NO: 506) and/or an epitope comprised within or overlapping with the sequence FGNMEGDCPSDWKTDSTCRM (SEQ ID NO: 518); and (bb) an epitope comprised within or overlapping with the sequence LLNENSYVPREAGSQKDENLAL (SEQ ID NO: 505) and/or an epitope comprised within or overlapping with the sequence LVENPGGYVAYSKAATVTGKL (SEQ ID NO: 520) and/or an epitope comprised within or overlapping with the sequence IYMDQTKFPIVNAELSF (SEQ ID NO: 521) and/or an epitope comprised within or overlapping with the sequence ISRAAAEKL (SEQ ID NO: 522) and/or an epitope comprised within or overlapping with the sequence VTSESKNVKLTVSNVLKE (SEQ ID NO: 523) and/or an epitope comprised within or overlapping with the sequence FCEDTDYPYLGTTMDT (SEQ ID NO: 524). In some embodiments, provided is an antigen-binding protein, wherein the antigen binding protein comprises an antibody or antigen-binding fragment thereof which binds to one or more epitopes of hTfR selected from: (a) an epitope consisting of the sequence LLNE (SEQ ID NO: 525) and/or an epitope consisting of the sequence TYKEL (SEQ ID NO: 507); (b) an epitope consisting of the sequence DSTDFTGT (SEQ ID NO: 526) and/or an epitope consisting of the sequence VKHPVTGQF (SEQ ID NO: 527) and/or an epitope consisting of the sequence IERIPEL (SEQ ID NO: 528); (c) an epitope consisting of the sequence LNENSYVPREAGSQKDEN (SEQ ID NO: 529); (d) an epitope consisting of the sequence FEDL (SEQ ID NO: 519); (e) an epitope consisting of the sequence IVDKNGRL (SEQ ID NO: 530); (f) an epitope consisting of the sequence IVDKNGRLVY (SEQ ID NO: 531); (g) an epitope consisting of the sequence DQTKF (SEQ ID NO: 532); (h) an epitope consisting of the sequence LVENPGGY (SEQ ID NO: 533) and/or an epitope consisting of the sequence PIVNAELSF (SEQ ID NO: 534) and/or an epitope consisting of the sequence PYLGTTMDT (SEQ ID NO: 535); (i) an epitope consisting of the sequence LLNENSYVPREAGSQKDENLAL (SEQ ID NO: 505) and/or an epitope consisting of the sequence IYMDQTKFPIVNAEL (SEQ ID NO: 506) and/or an epitope consisting of the sequence TYKEL (SEQ ID NO: 507); (j) an epitope consisting of the sequence KRKLSEKLDSTDFTGTIKL (SEQ ID NO: 508) and/or an epitope consisting of the sequence YTLIEKTMQNVKHPVTGQFL (SEQ ID NO: 509) and/or an epitope consisting of the sequence LIERIPELNKVARAAAE (SEQ ID NO: 510); (k) an epitope consisting of the sequence LNENSYVPREAGSQKDENL (SEQ ID NO: 511); (1) an epitope consisting of the sequence GTKKDFEDL (SEQ ID NO: 512); (m) an epitope consisting of the sequence SVIIVDKNGRLVYLVENPGGYVAYSK (SEQ ID NO: 513); (n) an epitope consisting of the sequence LLNENSYVPREAGSQKDEN (SEQ ID NO: 514) and/or an epitope consisting of the sequence DQTKFPIVNAEL (SEQ ID NO: 515) and/or an epitope consisting of the sequence TYKELIERIPELNK (SEQ ID NO: 516); (o) an epitope consisting of the sequence LLNENSYVPREAGSQKDEN (SEQ ID NO: 514) and/or an epitope consisting of the sequence TYKELIERIPELNK (SEQ ID NO: 516); (p) an epitope consisting of the sequence SVIIVDKNGRLVYLVENPGGYVAY (SEQ ID NO: 517); (q) an epitope consisting of the sequence IYMDQTKFPIVNAEL (SEQ ID NO: 506) and/or an epitope consisting of the sequence FGNMEGDCPSDWKTDSTCRM (SEQ ID NO: 518); and (r) an epitope consisting of the sequence LLNENSYVPREAGSQKDENLAL (SEQ ID NO: 505) and/or an epitope consisting of the sequence LVENPGGYVAYSKAATVTGKL (SEQ ID NO: 520) and/or an epitope consisting of the sequence IYMDQTKFPIVNAELSF (SEQ ID NO: 521) and/or an epitope consisting of the sequence ISRAAAEKL (SEQ ID NO: 522) and/or an epitope consisting of the sequence VTSESKNVKLTVSNVLKE (SEQ ID NO: 523) and/or an epitope consisting of the sequence FCEDTDYPYLGTTMDT (SEQ ID NO: 524).

An antigen is a molecule, such as a peptide (e.g., TfR or a fragment thereof (an antigenic fragment)), to which, for example, an antibody or antigen-binding fragment thereof binds. The specific region on an antigen that an antibody recognizes and binds to is called the epitope. Antigen-binding proteins (e.g., antibodies) described herein that specifically bind to such antigens are part of the present disclosure.

The term “epitope” refers to an antigenic determinant (e.g., on TfR) that interacts with a specific antigen-binding site of an antigen-binding protein, e.g., a variable region of an antibody, known as a paratope. A single antigen may have more than one epitope. Thus, different antibodies may bind to different areas on an antigen and may have different biological effects. The term “epitope” may also refer to a site on an antigen to which B and/or T cells respond and/or to a region of an antigen that is bound by an antibody. Epitopes may be defined as structural or functional. Functional epitopes are generally a subset of the structural epitopes and have those residues that directly contribute to the affinity of the interaction. Epitopes may be linear or conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes to which antigen-binding proteins described herein bind may be included in fragments of TfR, for example the extracellular domain thereof. Antigen-binding proteins (e.g., antibodies) described herein that bind to such epitopes are part of the present disclosure.

Methods for determining the epitope of an antigen-binding protein, e.g., antibody or fragment or polypeptide, include alanine scanning mutational analysis, peptide blot analysis (Reineke (2004) Methods Mol. Biol. 248: 443-63), peptide cleavage analysis, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antigen-binding protein (e.g., antibody or fragment or polypeptide) interacts is hydrogen/deuterium exchange detected by mass spectrometry. See, e.g., Ehring (1999) Analytical Biochemistry 267: 252-259; Engen and Smith (2001) Anal. Chem. 73: 256A-265A.

The present disclosure includes antigen-binding proteins that compete for binding to a TfR epitope as discussed herein, with an antigen-binding protein described herein. The term “competes” as used herein, refers to an antigen-binding protein (e.g., antibody or antigen-binding fragment thereof) that binds to an antigen (e.g., TfR) and inhibits or blocks the binding of another antigen-binding protein (e.g., antibody or antigen-binding fragment thereof) to the antigen. Unless otherwise stated, the term also includes competition between two antigen-binding proteins e.g., antibodies, in both orientations, i.e., a first antibody that binds antigen and blocks binding by a second antibody and vice versa. Thus, in an embodiment, competition occurs in one such orientation. In certain embodiments, the first antigen-binding protein (e.g., antibody) and second antigen-binding protein (e.g., antibody) may bind to the same epitope. Alternatively, the first and second antigen-binding proteins (e.g., antibodies) may bind to different, but, for example, overlapping or non-overlapping epitopes, wherein binding of one inhibits or blocks the binding of the second antibody, e.g., via steric hindrance. Competition between antigen-binding proteins (e.g., antibodies) may be measured by methods known in the art, for example, by a real-time, label-free bio-layer interferometry assay. Also, binding competition between TfR-binding proteins (e.g., monoclonal antibodies (mAbs)) can be determined using a real time, label-free bio-layer interferometry assay on an Octet RED384 biosensor (Pall ForteBio Corp.).

Typically, an antibody or antigen-binding fragment described herein which is modified in some way retains the ability to specifically bind to TfR, e.g., retains at least 10% of its TfR binding activity (when compared to the parental antibody) when that activity is expressed on a molar basis. Preferably, an antibody or antigen-binding fragment described herein retains at least 20%, 50%, 70%, 80%, 90%, 95% or 100% or more of the TfR binding affinity as the parental antibody. It is also intended that an antibody or antigen-binding fragment described herein may include conservative or non-conservative amino acid substitutions (referred to as “conservative variants” or “function conserved variants” of the antibody) that do not substantially alter its biologic activity.

An anti-TfR antigen-binding protein provided herein may be a monoclonal antibody or an antigen-binding fragment of a monoclonal antibody which may be tethered to a payload. Provided herein are monoclonal anti-TfR antigen-binding proteins, e.g., antibodies and antigen-binding fragments thereof, as well as monoclonal compositions comprising a plurality of isolated monoclonal antigen-binding proteins. The term “monoclonal antibody” or “mAb”, as used herein, refers to a member of a population of substantially homogeneous antibodies, i.e., the antibody molecules comprising the population are identical in amino acid sequence except for possible naturally occurring mutations that may be present in minor amounts. A “plurality” of such monoclonal antibodies and fragments in a composition refers to a concentration of identical (i.e., as discussed above, in amino acid sequence except for possible naturally occurring mutations that may be present in minor amounts) antibodies and fragments which is above that which would normally occur in nature, e.g., in the blood of a host organism such as a mouse or a human.

In an embodiment, an anti-TfR antigen-binding protein, e.g., antibody or antigen-binding fragment (which may be tethered to a payload) comprises a heavy chain constant domain, e.g., of the type IgA (e.g., IgA1 or IgA2), IgD, IgE, IgG (e.g., IgG1, IgG2, IgG3 and IgG4) or IgM. In an embodiment, an antigen-binding protein, e.g., antibody or antigen-binding fragment, comprises a light chain constant domain, e.g., of the type kappa or lambda. In an embodiment, a V_{H} as set forth herein is linked to a human heavy chain constant domain (e.g., IgG) and a V_{L} as set forth herein is linked to a human light chain constant domain (e.g., kappa). The present disclosure includes antigen-binding proteins comprising the variable domains set forth herein, which are linked to a heavy and/or light chain constant domain, e.g., as set forth herein.

Provided herein are human anti-TfR antigen-binding proteins which may be tethered to a payload. The term “human” antigen-binding protein, such as an antibody or antigen-binding fragment, as used herein, includes antibodies and fragments having variable and constant regions derived from human germline immunoglobulin sequences whether in a human cell or grafted into a non-human cell, e.g., a mouse cell. See e.g., U.S. Pat. Nos. 8,502,018, 6,596,541 or U.S. Pat. No. 5,789,215. The anti-TfR human mAbs provided herein may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse) have been grafted onto human FR sequences. The term includes antibodies recombinantly produced in a non-human mammal or in cells of a non-human mammal. The term is not intended to include natural antibodies directly isolated from a human subject. The present disclosure includes human antigen-binding proteins (e.g., antibodies or antigen-binding fragments thereof described herein).

Provided herein are anti-TfR chimeric antigen-binding proteins, e.g., antibodies and antigen-binding fragments thereof (which may be tethered to a payload), and methods of use thereof. As used herein, a “chimeric antibody” is an antibody having the variable domain from a first antibody and the constant domain from a second antibody, where the first and second antibodies are from different species. (see e.g., U.S. Pat. No. 4,816,567; and Morrison et al., (1984) Proc. Natl. Acad. Sci. USA 81: 6851-6855). The present disclosure includes chimeric antibodies comprising the variable domains which are set forth herein and a non-human constant domain.

The term “recombinant” anti-TfR antigen-binding proteins, such as antibodies or antigen-binding fragments thereof (which may be tethered to a payload), refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a cell (e.g., CHO cells) such as a cellular expression system or isolated from a recombinant combinatorial human antibody library. The present disclosure includes recombinant antigen-binding proteins, such as antibodies and antigen-binding fragments as set forth herein.

An antigen-binding fragment of an antibody will, in an embodiment, comprise less than a full antibody but still binds specifically to antigen, e.g., TfR, e.g., including at least one variable domain. The variable domain may be of any size or amino acid composition and will generally comprise at least one (e.g., 3) CDR(s), which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} and/or V_{L} domain which are bound non-covalently.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody described herein include: (i) V_{H}-CH1; (ii) V_{H}-CH2; (iii) V_{H}-CH3; (iv) V_{H}-CH1-CH2; (v) V_{H}-CH1-CH2-CH3; (vi) V_{H}-CH2-CH3; (vii) V_{H}-CL; (viii) V_{L}-CH1; (ix) V_{L}-CH2; (x) V_{L}-CH3; (xi) V_{L}-CH1-CH2; (xii) VL-CH1-CH2-CH3; (xiii) V_{L}-CH2-CH3; and (xiv) V_{L}-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody described herein may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)). The present disclosure includes an antigen-binding fragment of an antigen-binding protein such as an antibody set forth herein.

Antigen-binding proteins (e.g., antibodies and antigen-binding fragments) may be monospecific or multi-specific (e.g., bispecific). Multispecific antigen-binding proteins are discussed further herein. The present disclosure includes monospecific as well as multispecific (e.g., bispecific) antigen-binding fragments comprising one or more variable domains from an antigen-binding protein that is specifically set forth herein.

A “variant” of a polypeptide, such as an immunoglobulin chain, refers to a polypeptide comprising an amino acid sequence that is at least about 70-99.9% (e.g., at least 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein (e.g., any of SEQ ID NOs: 2; 3; 4; 5; 7; 8; 9; 10; 12; 13; 14; 15; 17; 18; 19; 20; 22; 23; 24; 25; 27; 28; 29; 30; 32; 33; 34; 35; 37; 38; 39; 40; 42; 43; 44; 45; 47; 48; 49; 50; 52; 53; 54; 55; 57; 58; 59; 60; 62; 63; 64; 65; 67; 68; 69; 70; 72; 73; 74; 75; 77; 78; 79; 80; 82; 83; 84; 85; 87; 88; 89; 90; 92; 93; 94; 95; 97; 98; 99; 100; 102; 103; 104; 105; 107; 108; 109; 110; 112; 113; 114; 115; 117; 118; 119; 120; 122; 123; 124; 125; 127; 128; 129; 130; 132; 133; 134; 135; 137; 138; 139; 140; 142; 143; 144; 145; 147; 148; 149; 150; 152; 153; 154; 155; 157; 158; 159; 160; 162; 163; 164; 165; 167; 168; 169; 170; 172; 173; 174; 175; 177; 178; 179; 180; 182; 183; 184; 185; 187; 188; 189; 190; 192; 193; 194; 195; 197; 198; 199; 200; 202; 203; 204; 205; 207; 208; 209; 210; 212; 213; 214; 215; 217; 218; 219; 220; 222; 223; 224; 225; 227; 228; 229; 230; 232; 233; 234; 235; 237; 238; 239; 240; 242; 243; 244; 245; 247; 248; 249; 250; 252; 253; 254; 255; 257; 258; 259; 260; 262; 263; 264; 265; 267; 268; 269; 270; 272; 273; 274; 275; 277; 278; 279; 280; 282; 283; 284; 285; 287; 288; 289; 290; 292; 293; 294; 295; 297; 298; 299; 300; 302; 303; 304; 305; 307; 488; 308; 309; 310; 312; 313; 314; 315; 317; 318; 319; 320; 321 (optionally not including the N-terminal MHRPRRRGTRPPPLALLAALLLAARGADA (SEQ ID NO: 500)), 322 (optionally not including the N-terminal MHRPRRRGTRPPPLALLAALLLAARGADA (SEQ ID NO: 500)), 323 (optionally not including the N-terminal MHRPRRRGTRPPPLALLAALLLAARGADA (SEQ ID NO: 500)), 324 (optionally not including the N-terminal MHRPRRRGTRPPPLALLAALLLAARGADA (SEQ ID NO: 500)); 328-423 or 427-458); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment) and/or comprising the amino acid sequence but having one or more (e.g., 1, 2, 3, 4, 5, 6, 7, 8, 9 or 10) mutations (e.g., point mutation, insertion, truncation, and/or deletion).

Moreover, a variant of a polypeptide may include a polypeptide such as an immunoglobulin chain which may include the amino acid sequence of the reference polypeptide whose amino acid sequence is specifically set forth herein but for one or more (e.g., 1, 2, 3, 4, 5, 6, 7, 8, 9 or 10) mutations, e.g., one or more missense mutations (e.g., conservative substitutions), non-sense mutations, deletions, or insertions. For example, the present disclosure includes TfR-binding proteins which include an immunoglobulin light chain (or V_{L}) variant comprising the amino acid sequence set forth in SEQ ID NO: 7, 17, 27, 37, 465, 47, 466, 57, 468, 67, 469, 77, 471, 87, 97, 107, 117, 474, 127, 137, 147, 476, 157, 167, 177, 187, 479, 197, 207, 217, 227, 237, 247, 257, 267, 277, 287, 297, 307, 488, 317, or 484 but having one or more of such mutations and/or an immunoglobulin heavy chain (or V_{H}) variant comprising the amino acid sequence set forth in SEQ ID NO: 2, 462, 12, 463, 22, 464, 32, 42, 52, 467, 62, 492, 72, 470, 82, 92, 472, 102, 112, 473, 122, 132, 142, 475, 152, 162, 477, 172, 182, 478, 192, 480, 202, 481, 212, 222, 232, 242, 252, 482, 262, 272, 282, 292, 302, 483, or 312 but having one or more of such mutations. In an embodiment, a TfR-binding protein includes an immunoglobulin light chain variant comprising CDR-L1, CDR-L2 and CDR-L3 wherein one or more (e.g., 1 or 2 or 3) of such CDRs has one or more of such mutations (e.g., conservative substitutions) and/or an immunoglobulin heavy chain variant comprising CDR-H1, CDR-H2 and CDR-H3 wherein one or more (e.g., 1 or 2 or 3) of such CDRs has one or more of such mutations (e.g., conservative substitutions).

The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

A “conservatively modified variant” or a “conservative substitution”, e.g., of an immunoglobulin chain set forth herein, refers to a variant wherein there is one or more substitutions of amino acids in a polypeptide with other amino acids having similar characteristics (e.g., charge, side-chain size, hydrophobicity/hydrophilicity, backbone conformation and rigidity, etc.). Such changes can frequently be made without significantly disrupting the biological activity of the antibody or fragment. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4^{th} Ed.)). In addition, substitutions of structurally or functionally similar amino acids are less likely to significantly disrupt biological activity. The present disclosure includes TfR-binding proteins comprising such conservatively modified variant immunoglobulin chains.

Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Alternatively, a conservative replacement is any change having a positive value in the PAM250 log-likelihood matrix disclosed in Gonnet et al. (1992) Science 256: 1443-45.

Antibodies and antigen-binding fragments described herein comprise immunoglobulin chains including the amino acid sequences specifically set forth herein (and variants thereof) as well as cellular and in vitro post-translational modifications to the antibody or fragment. For example, the present disclosure includes antibodies and antigen-binding fragments thereof that specifically bind to TfR comprising heavy and/or light chain amino acid sequences set forth herein as well as antibodies and fragments wherein one or more asparagine, serine and/or threonine residues is glycosylated, one or more asparagine residues is deamidated, one or more residues (e.g., Met, Trp and/or His) is oxidized, the N-terminal glutamine is pyroglutamate (pyroE) and/or the C-terminal lysine or other amino acid is missing.

In an embodiment, an anti-hTfR:Payload or anti-hTfR:Payload (e.g., in scFv, Fab, antibody or antigen-binding fragment thereof format), e.g., wherein the payload is human GAA, exhibits one or more of the following characteristics:
Affinity (K_{D}) for binding to human TfR at 25° C. in surface plasmon resonance format of about 41 nM or a higher affinity (e.g., about 1 or 0.1 nM or about 0.18 to about 1.2 nM, or higher);
Affinity (K_{D}) for binding to monkey TfR at 25° C. in surface plasmon resonance format of about 0 nM (no detectable binding) or a higher affinity (e.g., about 20 nM or higher);
Ratio of K_{D} for binding to monkey TfR/human TfR at 25° C. in surface plasmon resonance format of from 0 to 278 (e.g., about 17 or 18);
Blocks about 3, 5, 10 or 13% hTfR (e.g., Hmm-hTFRC such as REGN2431) binding to Human Holo-Tf when in Fab format (IgG1), e.g., no more than about 45% blocking;
Blocks about 6, 8, 10 or 13% hTfR (e.g., Hmm-hTFRC such as REGN2431) binding to Human Holo-Tf when in scFv (V_{K}-V_{H}) format, e.g., no more than about 45% blocking;
Blocks about 11, 17, 23 or 26% hTfR (e.g., Hmm-hTFRC such as REGN2431) binding to Human Holo-Tf when in scFv (V_{H}-V_{L}) format, e.g., no more than about 45% blocking;
Exhibits a ratio of about 1 or greater; 0.67 or greater; 1.08 or greater; 0.91 or greater; 0.65 or greater; 0.55 or greater; 0.50 or greater; 0.27 or greater; 0.72 or greater; 1.05 or greater; 0.49 or greater; 0.29 or greater; 1.29 or greater; 1.72 or greater; 1.79 or greater; 3.08 or greater; 1.24 or greater; 0.59 or greater; or 0.47 or greater (or about 1-2 or greater) mature hGAA protein in brain (normalized to that of positive control 8D3:GAA scFv) in mice (e.g., Tfrc^{hum/hum} knock-in mice) administered the molecule via HDD, when in anti-hTfR scFv:hGAA format; or delivers mature human GAA protein to the brain of humans administered said scFv:hGAA molecule;
Exhibits a ratio of about 0.44, 0.05, 1.13 or 0.60 (about 0.1-1.2) mature hGAA protein in brain parenchyma (normalized to that of positive control 8D3:GAA scFv) in mice (e.g., Tfrc^{hum}/hum knock-in mice) administered the molecule via HDD, when in anti-hTfR scFv:hGAA format; or delivers mature human GAA protein to the brain parenchyma of humans administered said scFv:hGAA molecule;
Exhibits a ratio of about 0.67, 1.80, 1.78 or 7.74 (about 1-2) mature hGAA protein in quadriceps (normalized to that of positive control 8D3:GAA scFv) in mice (e.g., Tfrc^{hum}/hum knock-in mice) administered the molecule via HDD, when in anti-hTfR scFv:hGAA format; or delivers mature human GAA protein to the quadricep or other muscle tissue of humans administered said scFv:hGAA molecule;
Exhibits a ratio of about 0.94, 0.49, 0.61 or 1.90 (about 0.1-1.2) mature hGAA protein in brain parenchyma (normalized to that of positive control 8D3:GAA scFv) in mice (e.g., Tfrc^{hum} knock-in mice) administered the molecule via AAV8 liver depot, when in anti-hTfR scFv:hGAA format; or delivers mature human GAA protein to the brain parenchyma of humans administered said scFv:hGAA molecule via viral, e.g., AAV, liver depot or parenterally delivered in protein scFv:hGAA fusion format;
Delivers mature hGAA protein to serum, liver, cerebrum, cerebellum, spinal cord, heart and/or quadricep in mice (e.g., Tfrc^{hum} knock-in mice) administered the molecule via AAV8 liver depot, when in anti-hTfR scFv:hGAA format; or delivers mature human GAA protein to the serum, liver, cerebrum, cerebellum, spinal cord, heart and/or quadricep of humans administered said scFv:hGAA molecule via viral, e.g., AAV, liver depot or parenterally delivered in protein scFv:hGAA fusion format;
Reduces glycogen stored in cerebrum, cerebellum, spinal cord, heart and/or quadricep in mice (e.g., Tfrc^{hum} knock-in mice) administered the molecule via AAV8 liver depot, when in anti-hTfR scFv:hGAA format; e.g., by at least 75% to greater than 95% or greater than 99%; or reduces glycogen stored in cerebrum, cerebellum, spinal cord, heart and/or quadricep of humans administered said scFv:hGAA molecule via viral, e.g., AAV, liver depot, or parenterally delivered in protein scFv:hGAA fusion format;
Reduces glycogen levels in tissues (e.g., cerebellum) of Gaa^{-/-}/Tfrc^{hum} mice treated with liver-depot AAV8 anti-hTFRC scfv:hGAA (e.g., 4e11vg/kg AAV8) by at least about 90% (e.g., about 95% or more) relative to untreated Gaa^{-/-}/Tfrc^{hum} mice;
Reduces glycogen levels in tissues (e.g., quadricep) of Gaa^{-/-}/Tfrc^{hum} mice treated with liver-depot AAV8 anti-hTFRC scfv:hGAA (e.g., 4e11vg/kg AAV8) by at least about 89% (e.g., about 90% or 91% or more) relative to untreated Gaa^{-/-}/Tfrc^{hum} mice; or of humans treated with the fusion, e.g., by parenteral deliver of the fusion protein;
Does not cause abnormal iron homeostasis when administered (e.g., by HDD or AAV8 episomal liver depot) to Tfrc^{hum} mice; e.g., wherein the mice maintain normal serum, heart, liver and/or spleen iron levels, normal total iron-binding capacity (TIBC), and/or normal hepcidin levels); or when administered to humans, e.g., by parenteral deliver of the fusion protein;
When chromosomally inserted (e.g., into the albumin gene locus) or delivered episomally to a subject (e.g., to a human or Gaa^{-/-}/Tfrc^{hum/hum} mouse), for example, in an AAV8 vector, DNA encoding the fusion causes expression of mature human GAA to serum, liver, cerebrum and/or quadricep; and/or
When chromosomally inserted (e.g., into the albumin gene locus) or delivered episomally (e.g., to a human or Gaa^{-/-}/Tfrc^{hum/hum} mouse), for example, in an AAV8 vector, DNA encoding the fusion reduces glycogen levels in the cerebrum and/or quadricep.
TfrC^{hum} or Tfrc^{hum/hum} are homozygous knock-in mice.

The amino acid sequences of domains in anti-human transferrin receptor antigen-binding proteins provided herein are summarized below in Table A. The amino acid sequences of domains in anti-human transferrin receptor antigen-binding proteins of fusions provided herein are also summarized below in Table A. For example, anti-human transferrin receptor 1 antibodies and antigen-binding fragments thereof (e.g., scFvs and Fabs) comprising the HCVR and LCVR of the molecules in Table A; or comprising the CDRs thereof, fused to a payload, are provided herein. In a specific example, the anti-human transferrin receptor 1 antibodies and antigen-binding fragments thereof (e.g., scFvs and Fabs) comprise the HCVR and LCVR of or comprise the CDRs of #23 or #25 in Table A. In a specific example, the anti-human transferrin receptor 1 antibodies and antigen-binding fragments thereof (e.g., scFvs and Fabs) comprise the HCVR and LCVR of or comprise the CDRs of #23 in Table A. In a specific example, the anti-human transferrin receptor 1 antibodies and antigen-binding fragments thereof (e.g., scFvs and Fabs) comprise the HCVR and LCVR of or comprise the CDRs of #14 in Table A. In a specific example, the anti-human transferrin receptor 1 antibodies and antigen-binding fragments thereof (e.g., scFvs and Fabs) comprise the HCVR and LCVR of or comprise the CDRs of #18 in Table A. In a specific example, the anti-human transferrin receptor 1 antibodies and antigen-binding fragments thereof (e.g., scFvs and Fabs) comprise the HCVR and LCVR of or comprise the CDRs of #27 in Table A. In a specific example, the anti-human transferrin receptor 1 antibodies and antigen-binding fragments thereof (e.g., scFvs and Fabs) comprise the HCVR and LCVR of or comprise the CDRs of #28 in Table A.

TABLE A
SEQ ID NOs of Domains in Antibodies, Antigen-Binding Fragments
(e.g., Fabs or scFv Molecules), or Fusion Proteins (anti-hTfR:Payload).

| | anti-hTfR: | | | | | | | | |
|---|---|---|---|---|---|---|---|---|---|
| | Payload | | | | | | | | |
| # | Molecule | HCVR | HCDR1 | HCDR2 | HCDR3 | LCVR | LCDR1 | LCDR2 | LCDR3 |
| 1 | 31874B | 2 or 462 | 3 | 4 | 5 | 7 | 8 | 9 | 10 |
| 2 | 31863B | 12 or 463 | 13 | 14 | 15 | 17 | 18 | 19 | 20 |
| 3 | 69348 | 22 or 464 | 23 | 24 | 25 | 27 | 28 | 29 | 30 |
| 4 | 69340 | 32 | 33 | 34 | 35 | 37 or 465 | 38 | 39 | 40 |
| 5 | 69331 | 42 | 43 | 44 | 45 | 47 or 466 | 48 | 49 | 50 |
| 6 | 69332 | 52 or 467 | 53 | 54 | 55 | 57 or 468 | 58 | 59 | 60 |
| 7 | 69326 | 62 or 492 | 63 | 64 | 65 | 67 or 469 | 68 | 69 | 70 |
| 8 | 69329 | 72 or 470 | 73 | 74 | 75 | 77 or 471 | 78 | 79 | 80 |
| 9 | 69323 | 82 | 83 | 84 | 85 | 87 | 88 | 89 | 90 |
| 10 | 69305 | 92 or 472 | 93 | 94 | 95 | 97 | 98 | 99 | 100 |
| 11 | 69307 | 102 | 103 | 104 | 105 | 107 | 108 | 109 | 110 |
| 12 | 12795B | 112 or 473 | 113 | 114 | 115 | 117 or 474 | 118 | 119 | 120 |
| 13 | 12798B | 122 | 123 | 124 | 125 | 127 | 128 | 129 | 130 |
| 14 | 12799B | 132 | 133 | 134 | 135 | 137 | 138 | 139 | 140 |
| 15 | 12801B | 142 or 475 | 143 | 144 | 145 | 147 or 476 | 148 | 149 | 150 |
| 16 | 12802B | 152 | 153 | 154 | 155 | 157 | 158 | 159 | 160 |
| 17 | 12808B | 162 or 477 | 163 | 164 | 165 | 167 | 168 | 169 | 170 |
| 18 | 12812B | 172 | 173 | 174 | 175 | 177 | 178 | 179 | 180 |
| 19 | 12816B | 182 or 478 | 183 | 184 | 185 | 187 or 479 | 188 | 189 | 190 |
| 20 | 12833B | 192 or 480 | 193 | 194 | 195 | 197 | 198 | 199 | 200 |
| 21 | 12834B | 202 or 481 | 203 | 204 | 205 | 207 | 208 | 209 | 210 |
| 22 | 12835B | 212 | 213 | 214 | 215 | 217 | 218 | 219 | 220 |
| 23 | 12847B | 222 | 223 | 224 | 225 | 227 | 228 | 229 | 230 |
| 24 | 12848B | 232 | 233 | 234 | 235 | 237 | 238 | 239 | 240 |
| 25 | 12843B | 242 | 243 | 244 | 245 | 247 | 248 | 249 | 250 |
| 26 | 12844B | 252 or 482 | 253 | 254 | 255 | 257 | 258 | 259 | 260 |
| 27 | 12845B | 262 | 263 | 264 | 265 | 267 | 268 | 269 | 270 |
| 28 | 12839B | 272 | 273 | 274 | 275 | 277 | 278 | 279 | 280 |
| 29 | 12841B | 282 | 283 | 284 | 285 | 287 | 288 | 289 | 290 |
| 30 | 12850B | 292 | 293 | 294 | 295 | 297 | 298 | 299 | 300 |
| 31 | 69261 | 302 or 483 | 303 | 304 | 305 | 307 or 488 | 308 | 309 | 310 |
| 32 | 69263 | 312 | 313 | 314 | 315 | 317 or 484 | 318 | 319 | 320 |

H31874B
HCVR (V_{H}) Nucleotide Sequence
(SEQ ID NO: 1)
GAGGTGCAGCTGGTGGAGTCTGGGGGAGGCTTGGTACAGCCTGGGGGGTCCCTGAGACTCTCCTGTGCAGCCTCTGG
ATTCGCCTTTAGCAGCTATGCCATGACCTGGGTCCGACAGGCTCCAGGGAAGGGGCTGGAGTGGGTCTCAGTTATCA
GTGGTACTGGTGGTAGTACATACTACGCAGACTCCGTGAAGGGCCGGTTCACCATCTCCAGAGACAATTCCAAGAAC
ACGCTGTATCTACAAATGAACAGCCTGAGAGCCGAGGACACGGCCGTATATTACTGTGCGAAAGGGGGAGCAGCTCG
TAGAATGGAATACTTCCAGTACTGGGGCCAGGGCACCCTGGTCACCGTCTCCTCA
HCVR (V_{H}) Amino Acid Sequence
(SEQ ID NO: 2)
EVQLVESGGGLVQPGGSLRLSCAASGFAFSSYAMTWVRQAPGKGLEWVSVISGTGGSTYYADSVKGRFTISRDNSKN
TLYLQMNSLRAEDTAVYYCAKGGAARRMEYFQYWGQGTLVTVSS
or
(SEQ ID NO: 462)
EVQLVESGGGLVQPGGSLRLSCAASGFAFSSYAMTWVRQAPGKGLEWVSVISGTGGSTYYADSVKGRFTISRDNSKN
TLYLQMNSLRAEDTAVYYCAKGGAARRMEYFQYWGQGTTVTVSS
HCDR1:
(SEQ ID NO: 3)
GFAFSSYA
HCDR2:
(SEQ ID NO: 4)
ISGTGGST
HCDR3:
(SEQ ID NO: 5)
AKGGAARRMEYFQY
LCVR (V_{L}) Nucleotide Sequence
(SEQ ID NO: 6)
GACATCCAGATGACCCAGTCTCCATCCTCCCTGTCTGCATCTGTAGGAGACAGAGTCACCATCACTTGCCGGGCGAG
TCAGGGCATTAGCAATTATTTAGCCTGGTATCAGCAGAAACCAGGGAAAGTTCCTAACCTCCTTATCTATGCTGCAT
CCACTTTGCAATCAGGGGTCCCATCTCGATTCAGTGGCAGTGGATCTGGGACAGATTTCACTCTCACCATCAGCAGC
CTGCAGCCTGAAGATGTTGCAACTTATTACTGTCAAAAGTATAACAGTGCCCCTCTCACTTTCGGCGGAGGGACCAA
GGTGGAGATCAAA
LCVR (V_{L}) Amino Acid Sequence
(SEQ ID NO: 7)
DIQMTQSPSSLSASVGDRVTITCRASQGISNYLAWYQQKPGKVPNLLIYAASTLQSGVPSRFSGSGSGTDFTLTISS
LQPEDVATYYCQKYNSAPLTFGGGTKVEIK
LCDR1:
(SEQ ID NO: 8)
QGISNY
LCDR2:
(SEQ ID NO: 9)
AAS
LCDR3:
(SEQ ID NO: 10)
QKYNSAPLT
31863B
HCVR (V_{H}) Nucleotide Sequence
(SEQ ID NO: 11)
GAGGTGCAGCTGGTGGAGTCTGGGGGAGGCTTGGTACAGCCTGGGGGGTCCCTGAGACTCTCCTGTGCAGCCTCTGG
ATTCACCTTTAACAGCTATGCCATGACCTGGGTCCGCCAGGCTCCAGGGAAGGGGCTGGAGTGGGTCTCATTTATTG
GTGGTAGTACTGGTAACACATACTACGCAGGCTCCGTGAAGGGCCGGTTCACCATCTCCAGCGACAATTCCAAGAAG
ACGCTGTATCTGCAAATGAACAGCCTGAGAGCCGAGGACACGGCCGTATATTACTGTGCGAAAGGGGGAGCAGCTCG
TAGAATGGAATACTTCCAGCACTGGGGCCAGGGCACCCTGGTCACCGTCTCCTCA
HCVR (V_{H}) Amino Acid Sequence
(SEQ ID NO: 12)
EVQLVESGGGLVQPGGSLRLSCAASGFTENSYAMTWVRQAPGKGLEWVSFIGGSTGNTYYAGSVKGRFTISSDNSKK
TLYLQMNSLRAEDTAVYYCAKGGAARRMEYFQHWGQGTLVTVSS
or
(SEQ ID NO: 463)
EVQLVESGGGLVQPGGSLRLSCAASGFTENSYAMTWVRQAPGKGLEWVSFIGGSTGNTYYAGSVKGRFTISSDNSKK
TLYLQMNSLRAEDTAVYYCAKGGAARRMEYFQHWGQGTTVTVSS
HCDR1:
(SEQ ID NO: 13)
GFTENSYA
HCDR2:
(SEQ ID NO: 14)
IGGSTGNT
HCDR3:
(SEQ ID NO: 15)
AKGGAARRMEYFQH
LCVR (V_{L}) Nucleotide Sequence
(SEQ ID NO: 16)
GACATCCAGATGACCCAGTCTCCATCCTCCCTGTCTGCATCTATAGGAGACAGAGTCACCATCACTTGCCGGGCGAG
TCAGGGCATTAGCAATTATTTAGCCTGGTATCAACAGAAACCAGGGAAAGTTCCTAAGCTCCTGATCTATGCTGCAT
CCACTTTGCAATCAGGGGTCCCATCTCGGTTCAGTGGCAGTGGATCTGGGACAGATTTCACTCTCACCATCAGCAGC
CTGCAGCCTGAAGATGTTGCAACTTATTACTGTCAAAACCATAACAGTGTCCCTCTCACTTTCGGCGGAGGGACCAA
GGTGGAGATCAAA
LCVR (V_{L}) Amino Acid Sequence
(SEQ ID NO: 17)
DIQMTQSPSSLSASIGDRVTITCRASQGISNYLAWYQQKPGKVPKLLIYAASTLQSGVPSRFSGSGSGTDFTLTISS
LQPEDVATYYCQNHNSVPLTFGGGTKVEIK
LCDR1:
(SEQ ID NO: 18)
QGISNY
LCDR2:
(SEQ ID NO: 19)
AAS
LCDR3:
(SEQ ID NO: 20)
QNHNSVPLT
69348
HCVR (V_{H}) Nucleotide Sequence
(SEQ ID NO: 21)
CAGGTGCAGCTGGTGGAGTCTGGGGGAGGCGTGGTCCAGCCTGGGAGGTCCCTGAGACTCTCCTGTGCAGCGTCTGG
ATTCACCTTCACTACCTATGGCATGCACTGGGTCCGCCAGGCTCCAGGCAAGGGGCTGGAGTGGGTGGCTGTTATAT
GGTATGATGGAAGTAATAAATATTATGGAGACTCCGTGAAGGGCCGATTCACCATCTCCAGAGACAATTCCAAGAAC
ACACTGTATCTGCAAATGAACAGCCTGAGAGTCGACGACACGGCTGTTTATTACTGTACGAGAACCCATGGCTATAC
CAGGTCGTCGGACGGTTTTGACTACTGGGGCCAGGGAACCCTGGTCACCGTCTCCTCA
HCVR (V_{H}) Amino Acid Sequence
(SEQ ID NO: 22)
QVQLVESGGGVVQPGRSLRLSCAASGFTFTTYGMHWVRQAPGKGLEWVAVIWYDGSNKYYGDSVKGRFTISRDNSKN
TLYLQMNSLRVDDTAVYYCTRTHGYTRSSDGFDYWGQGTLVTVSS
or
(SEQ ID NO: 464)
EVQLVESGGGVVQPGRSLRLSCAASGFTFTTYGMHWVRQAPGKGLEWVAVIWYDGSNKYYGDSVKGRFTISRDNSKN
TLYLQMNSLRVDDTAVYYCTRTHGYTRSSDGFDYWGQGTMVTVSS
HCDR1:
(SEQ ID NO: 23)
GFTFTTYG
HCDR2:
(SEQ ID NO: 24)
IWYDGSNK
HCDR3:
(SEQ ID NO: 25)
TRTHGYTRSSDGFDY
LCVR (V_{L}) Nucleotide Sequence
(SEQ ID NO: 26)
GACATCCAGATGACCCAGTCTCCATCCTCCCTGTCTGCATCTGTAGGAGACAGAGTCACCATCACTTGCCGGGCAAG
TCAGAGCATTAGAAATGTTTTAGGCTGGTTTCAGCAGAAACCAGGGAAAGCCCCTCAGCGCCTGATCTATGCTGCAT
CCAGTTTGCAAAGTGGGGTCCCATCAAGGTTCAGCGGCAGTGGATCTGGGACAGAATTCACTCTCACAATCAGCAGC
CTACAGCCTGAAGATTTTGCAACTTATTACTGTCTACAGCATAATTTTTACCCGCTCACTTTCGGCGGAGGGACCAA
GGTGGAGATCAAA
LCVR (V_{L}) Amino Acid Sequence
(SEQ ID NO: 27)
DIQMTQSPSSLSASVGDRVTITCRASQSIRNVLGWFQQKPGKAPQRLIYAASSLQSGVPSRESGSGSGTEFTLTISS
LQPEDFATYYCLQHNFYPLTFGGGTKVEIK
LCDR1:
(SEQ ID NO: 28)
QSIRNV
LCDR2:
(SEQ ID NO: 29)
AAS
LCDR3:
(SEQ ID NO: 30)
LQHNFYPLT
69340
HCVR (V_{H}) Nucleotide Sequence
(SEQ ID NO: 31)
GAAGTGCAGCTGGTGGAGTCTGGGGGAGGCTTGGTACAGCCTGGCAGGTCCCTGAGACTCTCCTGTGCAGCCTCTGG
ATTCACCTTTGATGATAAAGCCATGCACTGGGTCCGGCAAGTTCCAGGGAAGGGCCTGGAATGGATCTCAGGTATTA
GTTGGAATAGTGGTACTATAGGCTATGCGGACTCTGTGAAGGGCCGATTCATCATCTCCAGAGACAACGCCAAGAAC
TCCCTGTATCTACAAATGAACAGTCTGAGAGCTGAGGACACGGCCTTGTATTACTGCGCAAAAGATGGAGATACCAG
TGGCTGGTACTGGTACGGTTTGGACGTCTGGGGCCAAGGGACCACGGTCACCGTCTCCTCA
HCVR (V_{H}) Amino Acid Sequence
(SEQ ID NO: 32)
EVQLVESGGGLVQPGRSLRLSCAASGFTFDDKAMHWVRQVPGKGLEWISGISWNSGTIGYADSVKGRFIISRDNAKN
SLYLQMNSLRAEDTALYYCAKDGDTSGWYWYGLDVWGQGTTVTVSS
HCDR1:
(SEQ ID NO: 33)
GFTFDDKA
HCDR2:
(SEQ ID NO: 34)
ISWNSGTI
HCDR3:
(SEQ ID NO: 35)
AKDGDTSGWYWYGLDV
LCVR (V_{L}) Nucleotide Sequence
(SEQ ID NO: 36)
GAAATTGTGTTGACACAGTCTCCTGCCACCCTGTCTTTGTCTCCAGGGGAAAGAGCCACCCTCTCCTGCAGGGCCAG
TCAGAGTGTTAGCAGCTACTTAGCCTGGTACCAACAGAAACCTGGCCAGGCTCCCAGGCTCCTCATCCATGATGTAT
CCAACAGGGCCACTGGCATCCCAGCCAGGTTCAGTGGCAGTGGGTCTGGGACAGACTTCACTCTCACCATCAGCAGT
CTAGAGCCTGAAGATTTTGTAGTTTATTACTGTCAGCAGCGTAGCGACTGGCCCATCACCTTCGGCCAAGGGACACG
ACTGGAGATTAAA
LCVR (V_{L}) Amino Acid Sequence
(SEQ ID NO: 37)
EIVLTQSPATLSLSPGERATLSCRASQSVSSYLAWYQQKPGQAPRLLIHDVSNRATGIPARFSGSGSGTDFTLTISS
LEPEDFVVYYCOORSDWPITFGQGTRLEIK
OR
(SEQ ID NO: 465)
DIVMTQSPATLSLSPGERATLSCRASQSVSSYLAWYQQKPGQAPRLLIHDVSNRATGIPARFSGSGSGTDFTLTISS
LEPEDFVVYYCQQRSDWPITFGQGTRLEIK
LCDR1:
(SEQ ID NO: 38)
QSVSSY
LCDR2:
(SEQ ID NO: 39)
DVS
LCDR3:
(SEQ ID NO: 40)
QQRSDWPIT
69331
HCVR (V_{H}) Nucleotide Sequence
(SEQ ID NO: 41)
CAGGTGCAGCTGGTGGAGTCTGGGGGAGGCGTGGTCCAGCCTGGGAGGTCCCTGAGACTCTCCTGTATAGCCTCTGG
ATTCACCTTCAGTGTCTATGGCATTCACTGGGTCCGCCAGGCTCCAGGCAAGGGGCTGGAGTGGATGGCAGTAATAT
CACATGATGGAAATATTAAACACTATGCAGACTCCGTGAAGGGCCGATTCACCATCTCCAGAGACAATTCCAAGAAC
ACGCTGTATCTTCAAATTAACAGCCTGAGAACTGAGGACACGGCTGTGTATTACTGTGCGAAAGATACCTGGAACTC
CCTTGATACTTTTGATATCTGGGGCCAAGGGACAATGGTCACCGTCTCTTCA
HCVR (V_{H}) Amino Acid Sequence
(SEQ ID NO: 42)
QVQLVESGGGVVQPGRSLRLSCIASGFTFSVYGIHWVRQAPGKGLEWMAVISHDGNIKHYADSVKGRFTISRDNSKN
TLYLQINSLRTEDTAVYYCAKDTWNSLDTFDIWGQGTMVTVSS
HCDR1:
(SEQ ID NO: 43)
GFTFSVYG
HCDR2:
(SEQ ID NO: 44)
ISHDGNIK
HCDR3:
(SEQ ID NO: 45)
AKDTWNSLDTFDI
LCVR (V_{L}) Nucleotide Sequence
(SEQ ID NO: 46)
GACATCCAGTTGACCCAGTCTCCATCCTCCCTGTCTGCATCTGTAGGAGACAGAGTCACCATCACTTGCTGGGCCAG
TCAGGGCATTAGCAGTTATTTAGCCTGGTATCAGCAAAAACCAGGGAAAGCCCCTAAGCTCCTGATCTATGCTGCAT
CCACTTTGCAAAGTGGGGTCCCATCAAGGTTCAGCGGCAGTGGATCTGGGACAGAATTCACTCTCACAATCAGCAGC
CTGCAGCCTGAAGATTTTGCAACTTATTACTGTCAACAGCTTAATAGTTACCCTCTCACTTTCGGCGGAGGGACCAA
GGTGGAGATCAAA
LCVR (V_{L}) Amino Acid Sequence
(SEQ ID NO: 47)
DIQLTQSPSSLSASVGDRVTITCWASQGISSYLAWYQQKPGKAPKLLIYAASTLQSGVPSRFSGSGSGTEFTLTISS
LQPEDFATYYCQQLNSYPLTFGGGTKVEIK
or
(SEQ ID NO: 466)
DIQMTQSPSSLSASVGDRVTITCWASQGISSYLAWYQQKPGKAPKLLIYAASTLQSGVPSRFSGSGSGTEFTLTISS
LQPEDFATYYCQQLNSYPLTFGGGTKVEIK
LCDR1:
(SEQ ID NO: 48)
QGISSY
LCDR2:
(SEQ ID NO: 49)
AAS
LCDR3:
(SEQ ID NO: 50)
QQLNSYPLT
69332
HCVR (V_{H}) Nucleotide Sequence
(SEQ ID NO: 51)
CAGGTCACCTTGAGGGAGTCTGGTCCCGCGCTGGTGAAACCCTCACAGACCCTCACACTGACCTGCACCTTCTCTGG
ATTCTCACTCAACACTTATGGGATGTTTGTGAGCTGGATCCGTCAGCCTCCAGGGAAGGCCCTAGAGTGGCTTGCAC
ACATTCATTGGGATGATGATAAATACTACAGCACATCTCTGAAGACCAGGCTCACCATCTCCAAGGACACCTCCAAA
AACCAGGTGGTCCTTACAATGACCAACATGGACCCTGTGGACACAGCCACGTATTATTGTGCACGGGGGCACAATAA
TTTGAACTACATCATCCACTGGGGCCAGGGAACCCTGGTCACCGTCTCCTCA
HCVR (V_{H}) Amino Acid Sequence
(SEQ ID NO: 52)
QVTLRESGPALVKPSQTLTLTCTFSGFSLNTYGMFVSWIRQPPGKALEWLAHIHWDDDKYYSTSLKTRLTISKDTSK
NQVVLTMTNMDPVDTATYYCARGHNNLNYIIHWGQGTLVTVSS
or
(SEQ ID NO: 467)
QVQLVESGPALVKPSQTLTLTCTFSGFSLNTYGMFVSWIRQPPGKALEWLAHIHWDDDKYYSTSLKTRLTISKDTSK
NQVVLTMTNMDPVDTATYYCARGHNNLNYIIHWGQGTLVTVSS
HCDR1:
(SEQ ID NO: 53)
GFSLNTYGMF
HCDR2:
(SEQ ID NO: 54)
IHWDDDK
HCDR3:
(SEQ ID NO: 55)
ARGHNNLNYIIH
LCVR (V_{L}) Nucleotide Sequence
(SEQ ID NO: 56)
GCCATCCAGATGACCCAGTCTCCATCCTCCCTGTCTGCATCTGTAGGAGACAGAGTCACCATCACTTGCCGGGCAAG
TCAGGGCATTAGAAATGATTTAGGCTGGTATCAGCAGAAACCAGGGAAAGCCCCTAAGCTCCTGATCTATGCTGCAT
CCACTTTACAAAGTGGGGTCCCATCAAGGTTCAGCGGCAGTGGATCTGGCACAGATTTCACTCTCACCATCAGCAGC
CTGCAGCCTGAAGATTTTGCAACTTATTACTGTCTACAAGATTACAATTACCCATTCACTTTCGGCCCTGGGACCAA
AGTGGATATCAAA
LCVR (V_{L}) Amino Acid Sequence
(SEQ ID NO: 57)
AIQMTQSPSSLSASVGDRVTITCRASQGIRNDLGWYQQKPGKAPKLLIYAASTLQSGVPSRFSGSGSGTDFTLTISS
LQPEDFATYYCLQDYNYPFTFGPGTKVDIK
or
(SEQ ID NO: 468)
DILMTQSPSSLSASVGDRVTITCRASQGIRNDLGWYQQKPGKAPKLLIYAASTLQSGVPSRFSGSGSGTDFTLTISS
LQPEDFATYYCLQDYNYPFTFGPGTKVEIK
LCDR1:
(SEQ ID NO: 58)
QGIRND
LCDR2:
(SEQ ID NO: 59)
AAS
LCDR3:
(SEQ ID NO: 60)
LQDYNYPFT
69326
HCVR (V_{H}) Nucleotide Sequence
(SEQ ID NO: 61)
GAGGTGCAGCTGGTGGAGTCTGGGGGAGGCTTGGTACAGCCTGGAGGGTCCCTGAGACTCTCCTGTGCAGTCTCTGG
ATTCATCTTCAGTAGTTATGAAATGAACTGGGTCCGCCAGGCTCCAGGGAAGGGGCTGGAGTGGGTTTCATACATTA
GTAGTAGTGGTAGTACCATATTCTACGCAGACTCTGTGAAGGGCCGATTCACCATCTCCAGAGACAACGCCAAGAAC
TCACTGTATCTGCAAATGAACAGCCTGAGAGCCGAGGACACGGCTGTTTATTACTGTGTGTCTGGAGTGGTCCTTTT
TGATGTCTGGGGCCAAGGGACAATGGTCACCGTCTCTTCA
HCVR (V_{H}) Amino Acid Sequence
(SEQ ID NO: 62)
EVQLVESGGGLVQPGGSLRLSCAVSGFIFSSYEMNWVRQAPGKGLEWVSYISSSGSTIFYADSVKGRFTISRDNAKN
SLYLQMNSLRAEDTAVYYCVSGVVLFDVWGQGTMVTVSS
or
(SEQ ID NO: 492)
QVQLVESGGGLVQPGGSLRLSCAVSGFIFSSYEMNWVRQAPGKGLEWVSYISSSGSTIFYADSVKGRFTISRDNAKN
SLYLQMNSLRAEDTAVYYCVSGVVLFDVWGQGTMVTVSS
HCDR1:
(SEQ ID NO: 63)
GFIFSSYE
HCDR2:
(SEQ ID NO: 64)
ISSSGSTI
HCDR3:
(SEQ ID NO: 65)
VSGVVLFDV
LCVR (V_{L}) Nucleotide Sequence
(SEQ ID NO: 66)
GAAATAGTGATGACGCAGTCTCCAGCCACCCTGTCTGTGTCTCCGGGGGAAAGAGCCACCCTCTCCTGCAGGGCCAG
TCAGAGTGTTAGCAGCAACTTTGCCTGGTACCAACAGAAACCTGGCCAGGCTCCCAGGCTCCTCATCTATAGTGCAT
CCTCCAGGGCCACTGGTATCCCAGTCAGGTTCAGTGGCAGTGGGTCTGGGACAGAGTTCACTCTCACCATCAGCAGC
CTGCAGTCTGAAGATTTTGCAGTTTATTACTGTCAGCAGTATAATATCTGGCCTCGGACGTTCGGCCAAGGGACCAA
GGTGGAAATCAAA
LCVR (V_{L}) Amino Acid Sequence
(SEQ ID NO: 67)
EIVMTQSPATLSVSPGERATLSCRASQSVSSNFAWYQQKPGQAPRLLIYSASSRATGIPVRFSGSGSGTEFTLTISS
LQSEDFAVYYCQQYNIWPRTFGQGTKVEIK
or
(SEQ ID NO: 469)
DIVMTQSPATLSVSPGERATLSCRASQSVSSNFAWYQQKPGQAPRLLIYSASSRATGIPVRFSGSGSGTEFTLTISS
LQSEDFAVYYCQQYNIWPRTFGQGTKVEIK
LCDR1:
(SEQ ID NO: 68)
QSVSSN
LCDR2:
(SEQ ID NO: 69)
SAS
LCDR3:
(SEQ ID NO: 70)
QQYNIWPRT
69329
HCVR (V_{H}) Nucleotide Sequence
(SEQ ID NO: 71)
GAGGTGCAGCTGGTGGAGTCTGGGGGAGGCTTGGTCCAGCCTGGGGGGTCCCTGAGACTCTCCTGTGCAGCCTCTGG
ATTCACCTTTAGTAACTATTGGATGACCTGGGTCCGCCAGGCTCCAGGGAAGGGGCTGGAGTGGGTGGCCAACATAA
AGGAAGATGGAAGTGAGAAAGACTATGTGGACTCTGTGAAGGGCCGATTCACCATCTCCAGAGACAACGCCAAGAAC
TCACTGTATCTGCAAATGAACAGCCTGAGAGGCGAGGACACGGCTGTGTATTACTGTGCGAGAGATGGGGAGCAGCT
CGTCGATTACTACTACTACTACGTTATGGACGTCTGGGGCCAAGGGACCACGGTCACCGTCTCCTCA
HCVR (V_{H}) Amino Acid Sequence
(SEQ ID NO: 72)
EVQLVESGGGLVQPGGSLRLSCAASGFTFSNYWMTWVRQAPGKGLEWVANIKEDGSEKDYVDSVKGRFTISRDNAKN
SLYLQMNSLRGEDTAVYYCARDGEQLVDYYYYYVMDVWGQGTTVTVSS
or
(SEQ ID NO: 470)
QVQLVESGGGLVQPGGSLRLSCAASGFTFSNYWMTWVRQAPGKGLEWVANIKEDGSEKDYVDSVKGRFTISRDNAKN
SLYLQMNSLRGEDTAVYYCARDGEQLVDYYYYYVMDVWGQGTTVTVSS
HCDR1:
(SEQ ID NO: 73)
GFTFSNYW
HCDR2:
(SEQ ID NO: 74)
IKEDGSEK
HCDR3:
(SEQ ID NO: 75)
ARDGEQLVDYYYYYVMDV
LCVR (V_{L}) Nucleotide Sequence
(SEQ ID NO: 76)
GACATCCAGATGACCCAGTCTCCATCTTCCGTGTCTGCATCTGTAGGAGACAGAGTCACCATCACTTGTCGGGCGAG
TCAGGGTATTAGCAGCTGGTTAGCCTGGTATCAGCAGAAACCAGGGAAAGCCCCTAAGCTCCTGATCTATGCTGCAT
CCAGTTTGCAAAGTGGGGTCCCATCAAGGTTCAGCGGCAGTGGATCTGGGACAGATTTCACTCTCACCATCAGCAGC
CTGCAGCCTGAAGATTTTGCAACTTACTATTGTCAAAAGGCTAACAGTTTCCCGTACACTTTTGGCCAGGGGACCAA
GCTGGAGATCAAA
LCVR (V_{L}) Amino Acid Sequence
(SEQ ID NO: 77)
DIQMTQSPSSVSASVGDRVTITCRASQGISSWLAWYQQKPGKAPKLLIYAASSLQSGVPSRESGSGSGTDFTLTISS
LQPEDFATYYCQKANSFPYTFGQGTKLEIK
or
(SEQ ID NO: 471)
DIQMTQSPSSVSASVGDRVTITCRASQGISSWLAWYQQKPGKAPKLLIYAASSLQSGVPSRFSGSGSGTDFTLTISS
LQPEDFATYYCQKANSFPYTFGQGTKVEIK
LCDR1:
(SEQ ID NO: 78)
QGISSW
LCDR2:
(SEQ ID NO: 79)
AAS
LCDR3:
(SEQ ID NO: 80)
QKANSFPYT
69323 (REGN16816 anti-hTfR scFv:hGAA)
HCVR (V_{H}) Nucleotide Sequence
(SEQ ID NO: 81)
GAAGTGCAGCTGGTGGAGTCTGGGGGAGGCTTGGTACAGCCTGGCAGGTCCCTGAGACTCTCCTGTGCAGCCTCTGG
ATTCACCTTTGATGACTATGCCATGCACTGGGTCCGGCAAGCTCCAGGGAAGGGCCTGGAGTGGGTCTCAGGTATTA
GTTGGAATAGTGGTTACATAGGCTATGCGGACTCTGTGAAGGGCCGATTCACCATCTCCAGAGACAACGCCGAGAAC
TCCCTACATCTGCAAATGAACAGTCTGAGAGCTGAGGACACGGCCTTGTATTACTGTGCAAGAGGGGGATCTACTCT
GGTTCGGGGAGTTAAGGGAGGCTACTACGGTATGGACGTCTGGGGCCAAGGGACCACGGTCACCGTCTCCTCA
HCVR (V_{H}) Amino Acid Sequence
(SEQ ID NO: 82)
EVQLVESGGGLVQPGRSLRLSCAASGFTEDDYAMHWVRQAPGKGLEWVSGISWNSGYIGYADSVKGRFTISRDNAEN
SLHLQMNSLRAEDTALYYCARGGSTLVRGVKGGYYGMDVWGQGTTVTVSS
HCDR1:
(SEQ ID NO: 83)
GFTEDDYA
HCDR2:
(SEQ ID NO: 84)
ISWNSGYI
HCDR3:
(SEQ ID NO: 85)
ARGGSTLVRGVKGGYYGMDV
LCVR (V_{L}) Nucleotide Sequence
(SEQ ID NO: 86)
GACATCCAGATGACCCAGTCTCCATCCTCCCTGTCTGCATCTGTAGGAGACAGAGTCACCATCACTTGCCGGGCAAG
TCAGAGCATAAGTAGCTATTTAAATTGGTATCAGCAGAAACCAGGTAAAGCCCCTAAGGTCCTGATCTATGCTGCAT
CCAGTTTGCAAAGTGGGGTCCCATCAAGGTTCAGTGGCAGTGGATCTGGGACAGATTTCACTCTCACCATCAGCAGT
CTGCAACCTGAAGATTTTGCAACTTACTACTGTCAACAGAGTTACAGTATTCCGCTCACTTTCGGCGGAGGGACCAA
GGTGGAGATCAAA
LCVR (V_{L}) Amino Acid Sequence
(SEQ ID NO: 87)
DIQMTQSPSSLSASVGDRVTITCRASQSISSYLNWYQQKPGKAPKVLIYAASSLQSGVPSRFSGSGSGTDFTLTISS
LQPEDFATYYCQQSYSIPLTFGGGTKVEIK
LCDR1:
(SEQ ID NO: 88)
QSISSY
LCDR2:
(SEQ ID NO: 89)
AAS
LCDR3:
(SEQ ID NO: 90)
QQSYSIPLT
69305
HCVR (V_{H}) Nucleotide Sequence
(SEQ ID NO: 91)
CAGGTGCAGCTGGTGGAGTCTGGGGGAGGCGTGGTCCAGCCTGGGAGGTCCCTGAGACTCTCCTGTGCAGCGTCTGG
ATTCACCTTCAGTAGCTATGGCATGCACTGGGTCCGCCAGGCTCCAGGCAAGGGGCTGGAGTGGGTGGCAGTTATAT
GGTATGATGGAAGTAATAAATACTATGCAGACTCCGTGAAGGGCCGATTCACCATCTCCAGAGACATTTCCAAGAAC
ACGCTGTATCTGCAAATGAACAGCCTGAGAGCCGAGGACACGGCCGTATATTACTGTGCGGGTCAACTGGATCTCTT
CTTTGACTACTGGGGCCAGGGAACCCTGGTCACCGTCTCCTCA
HCVR (V_{H}) Amino Acid Sequence
(SEQ ID NO: 92)
QVQLVESGGGVVQPGRSLRLSCAASGFTFSSYGMHWVRQAPGKGLEWVAVIWYDGSNKYYADSVKGRFTISRDISKN
TLYLQMNSLRAEDTAVYYCAGQLDLFFDYWGQGTLVTVSS
or
(SEQ ID NO: 472)
EVQLVESGGGVVQPGRSLRLSCAASGFTFSSYGMHWVRQAPGKGLEWVAVIWYDGSNKYYADSVKGRFTISRDISKN
TLYLQMNSLRAEDTAVYYCAGOLDLFFDYWGQGTLVTVSS
HCDR1:
(SEQ ID NO: 93)
GFTFSSYG
HCDR2:
(SEQ ID NO: 94)
IWYDGSNK
HCDR3:
(SEQ ID NO: 95)
AGQLDLFFDY
LCVR (V_{L}) Nucleotide Sequence
(SEQ ID NO: 96)
GACATCCAGATGACCCAGTCTCCATCCTCCCTGTCTGCATCTGTAGGAGACAGAGTCACCATCACTTGCCGGGCAAG
TCAGAGCATTGACAGGTATTTAAATTGGTATCGGCAGAAACCAGGGAAAGCCCCTAAGCTCCTGATCTATACTACAT
CCAGTTTGCAAAGTGGGGTCCCATCAAGGTTCAGTGGCAGTGGATCTGGGACAGATTTCACTCTCACCCTCAGCAGT
CTGCAGCCTGAAGATTTTGCAACTTACTACTGTCAGCAGAGTTACAGTCCCCCGCTCACTTTCGGCGGAGGGACCAA
GGTGGAGATCAAA
LCVR (V_{L}) Amino Acid Sequence
(SEQ ID NO: 97)
DIQMTQSPSSLSASVGDRVTITCRASQSIDRYLNWYROKPGKAPKLLIYTTSSLQSGVPSRFSGSGSGTDFTLTLSS
LQPEDFATYYCQQSYSPPLTFGGGTKVEIK
LCDR1:
(SEQ ID NO: 98)
QSIDRY
LCDR2:
(SEQ ID NO: 99)
TTS
LCDR3:
(SEQ ID NO: 100)
QQSYSPPLT
69307 (REGN16817 anti-hTfR scFv:hGAA)
HCVR (V_{H}) Nucleotide Sequence
(SEQ ID NO: 101)
GAGGTGCAGCTGGTGGAGTCTGGGGGAGGCTTGGTCCAGCCTGGGGGGTCCCTGAGACTCTCCTGTACAGCCTCTGG
ATTCACCTTTAGTAACTATTGGATGACCTGGGTCCGCCAGGCTCCAGGGAAGGGGCTGGAGTGGGTGGCCAACATAA
AGGAAGATGGAAGTGAGAAAGAGTATGTGGACTCTGTGAAGGGCCGGTTCACCATCTCCAGAGACAACGCCAAGAAT
TCACTGTATCTGCAAATGAACAGCCTGAGAGGCGAGGACACGGCTGTATATTACTGTGCGAGAGATGGGGAGCAGCT
CGTCGATTACTATTACTACTACGTTATGGACGTCTGGGGCCAAGGGACCACGGTCACCGTCTCCTCA
HCVR (V_{H}) Amino Acid Sequence
(SEQ ID NO: 102)
EVQLVESGGGLVQPGGSLRLSCTASGFTFSNYWMTWVRQAPGKGLEWVANIKEDGSEKEYVDSVKGRFTISRDNAKN
SLYLQMNSLRGEDTAVYYCARDGEQLVDYYYYYVMDVWGQGTTVTVSS
HCDR1:
(SEQ ID NO: 103)
GFTFSNYW
HCDR2:
(SEQ ID NO: 104)
IKEDGSEK
HCDR3:
(SEQ ID NO: 105)
ARDGEQLVDYYYYYVMDV
LCVR (V_{L}) Nucleotide Sequence
(SEQ ID NO: 106)
GACATCCAGATGACCCAGTCTCCATCTTCCGTGTCTGCATCTGTTGGAGACAGAGTCACCATCACTTGTCGGGCGAG
TCAGGGTATTAGCAGCTGGTTAGCCTGGTATCAGCAGAAACCAGGGAAAGCCCCTAAGCTCCTGATCTATGCTGCAT
CCAGTTTGCAAAGTGGGGTCCCATCAAGGTTCAGCGGCAGTGGATCTGGGACAGATTTCACTCTCACCATCAGCAGC
CTGCAGCCTGAAGATTTTGCAACTTACTATTGTCAAAAGGCTGACAGTCTCCCGTACGCTTTTGGCCAGGGGACCAA
GCTGGAGATCAAA
LCVR (V_{L}) Amino Acid Sequence
(SEQ ID NO: 107)
DIQMTQSPSSVSASVGDRVTITCRASQGISSWLAWYQQKPGKAPKLLIYAASSLQSGVPSRFSGSGSGTDFTLTISS
LQPEDFATYYCQKADSLPYAFGQGTKLEIK
LCDR1:
(SEQ ID NO: 108)
QGISSW
LCDR2:
(SEQ ID NO: 109)
AAS
LCDR3:
(SEQ ID NO: 110)
QKADSLPYA
12795B
HCVR (V_{H}) Nucleotide Sequence
(SEQ ID NO: 111)
GAGGTGCAGCTGGTGGAGTCTGGGGGAGGCTTGGTTCAGCCTGGGGGGTCCCTGAGACTCTCCTGTGCAACCTCTGG
ATTCACCTTTACCAGCTATGACATGAAGTGGGTCCGCCAGGCTCCAGGGCTGGGCCTGGAGTGGGTCTCAGCTATTA
GTGGTAGTGGTGGTAACACATACTACGCAGACTCCGTGAAGGGCCGGTTCACCATCTCCAGAGACAATTCCAGGAAC
ACGCTGTATCTGCAAATGAACAGCCTGAGAGCCGAGGACACGGCCGTATATTACTGTACGAGGTCCCATGACTTCGG
TGCCTTCGACTACTTTGACTACTGGGGCCAGGGAACCCTGGTCACCGTCTCCTCA
HCVR (V_{H}) Amino Acid Sequence
(SEQ ID NO: 112)
EVQLVESGGGLVQPGGSLRLSCATSGFTFTSYDMKWVRQAPGLGLEWVSAISGSGGNTYYADSVKGRFTISRDNSRN
TLYLQMNSLRAEDTAVYYCTRSHDFGAFDYFDYWGQGTLVTVSS
or
(SEQ ID NO: 473)
EVQLVQSGGGLVQPGGSLRLSCATSGFTFTSYDMKWVRQAPGLGLEWVSAISGSGGNTYYADSVKGRFTISRDNSRN
TLYLQMNSLRAEDTAVYYCTRSHDFGAFDYFDYWGQGTMVTVSS
HCDR1:
(SEQ ID NO: 113)
GFTFTSYD
HCDR2:
(SEQ ID NO: 114)
ISGSGGNT
HCDR3:
(SEQ ID NO: 115)
TRSHDFGAFDYEDY
LCVR (V_{L}) Nucleotide Sequence
(SEQ ID NO: 116)
GACATCCAGATGACCCAGTCTCCATCCTCCCTGTCTGCATCTGTGGGAGACAGAGTCACCATCACTTGCCGGGCAAG
TCAGGGCATTAGAGATCATTTTGGCTGGTATCAGCAGAAACCAGGGAAAGCCCCTAAGCGCCTGATCTATGCTGCAT
CCAGTTTGCACAGTGGGGTCCCATCAAGGTTCAGCGGCAGTGGATCTGGGACAGAATTCACTCTCACAATCAGCAGC
TTGCAGCCTGAAGATTTTGCAACCTATTACTGTCTACAGTATGATACTTACCCGCTCACTTTCGGCGGAGGGACCAA
GGTGGAGATCAAA
LCVR (V_{L}) Amino Acid Sequence
(SEQ ID NO: 117)
DIQMTQSPSSLSASVGDRVTITCRASQGIRDHFGWYQQKPGKAPKRLIYAASSLHSGVPSRFSGSGSGTEFTLTISS
LQPEDFATYYCLQYDTYPLTFGGGTKVEIK
or
(SEQ ID NO: 474)
DIQLTQSPSSLSASVGDRVTITCRASQGIRDHFGWYQQKPGKAPKRLIYAASSLHSGVPSRFSGSGSGTEFTLTISS
LQPEDFATYYCLQYDTYPLTFGGGTKVEIK
LCDR1:
(SEQ ID NO: 118)
QGIRDH
LCDR2:
(SEQ ID NO: 119)
AAS
LCDR3:
(SEQ ID NO: 120)
LQYDTYPLT
12798B (REGN17078 Fab; REGN17072 scFv; REGN16818 anti-hTfR scFv:hGAA)
HCVR (V_{H}) Nucleotide Sequence
(SEQ ID NO: 121)
GAAGTGCAGCTGGTGGAGTCTGGGGGAGACTTGGTACAGCCTGGCAGGTCCCTGAGACTCTCCTGTGCAGCCTCTGG
ATTCACCTTTGATGATTATGCCATGCACTGGGTCCGGCAAGCTCCAGGGAAGGGCCTGGAGTGGGTCTCAGGTATTA
GTTGGAATAGTGCTACCAGAGTCTATGCGGACTCTGTGAAGGGCCGATTCACCATCTCCAGAGACAACGCCAAGAAT
TTCCTGTATCTGCAAATGAACAGTCTGAGATCTGAGGACACGGCCTTGTATCACTGTGCAAAAGATATGGATATCTC
GCTAGGGTACTACGGTTTGGACGTCTGGGGCCAAGGGACCACGGTCACCGTCTCCTCA
HCVR (V_{H}) Amino Acid Sequence
(SEQ ID NO: 122)
EVQLVESGGDLVQPGRSLRLSCAASGFTFDDYAMHWVRQAPGKGLEWVSGISWNSATRVYADSVKGRFTISRDNAKN
FLYLQMNSLRSEDTALYHCAKDMDISLGYYGLDVWGQGTTVTVSS
HCDR1:
(SEQ ID NO: 123)
GFTEDDYA
HCDR2:
(SEQ ID NO: 124)
ISWNSATR
HCDR3:
(SEQ ID NO: 125)
AKDMDISLGYYGLDV
LCVR (V_{L}) Nucleotide Sequence
(SEQ ID NO: 126)
GAAATAGTGATGACGCAGTCTCCAGCCACCCTGTCTGTGTCTCCAGGGGAAAGAGCCACCCTCTCCTGCAGGGCCAG
TCAGACTGTTAGCAGCAACTTAGCCTGGTATCAGCAGAAACCTGGCCAGGCTCCCAGGCTCCTCATCTATGGTTCAT
CCTCCAGGGCCACTGGTATCCCAGCCAGGTTCAGTGGCAGTGGGTCTGGGACAGAGTTCACTCTCACCATCAGCAGC
CTGCAGTCTGAAGATTTTGCAGTTTATTACTGTCAGCAGTATAATAACTGGCCTCCCTACACTTTTGGCCAGGGGAC
CAAGCTGGAGATCAAA
LCVR (V_{L}) Amino Acid Sequence
(SEQ ID NO: 127)
EIVMTQSPATLSVSPGERATLSCRASQTVSSNLAWYQQKPGQAPRLLIYGSSSRATGIPARFSGSGSGTEFTLTISS
LOSEDFAVYYCQQYNNWPPYTFGQGTKLEIK
LCDR1:
(SEQ ID NO: 128)
QTVSSN
LCDR2:
(SEQ ID NO: 129)
GSS
LCDR3:
(SEQ ID NO: 130)
QQYNNWPPYT
12799B (REGN17079 Fab; REGN17073 scFv; REGN16819 anti-hTfR scFv:hGAA)
HCVR (V_{H}) Nucleotide Sequence
(SEQ ID NO: 131)
CAGATCACCTTGAAGGAGTCTGGTCCTACGCTGGTGAAACCCACACAGACCCTCACGCTGACCTGCACCTTCTCTGG
GTTCTCACTCAGCACTAGTGGAGTGGGTGTGGTCTGGATCCGTCAGCCCCCCGGAAAGGCCCTGGAGTGGCTTGCAC
TCATTTATTGGAATGATCATAAGCGGTACAGCCCATCTCTGGGGAGCAGGCTCACCATCACCAAGGACACCTCCAAA
AACCAGGTGGTCCTTACAATGACCAACATGGACCCTGTGGACACAGCCACATATTACTGTGCACACTACAGTGGGAG
CTATTCCTACTACTACTATGGTTTGGACGTCTGGGGCCAAGGGACCACGGTCACCGTCTCCTCA
HCVR (V_{H}) Amino Acid Sequence
(SEQ ID NO: 132)
QITLKESGPTLVKPTQTLTLTCTFSGFSLSTSGVGVVWIRQPPGKALEWLALIYWNDHKRYSPSLGSRLTITKDTSK
NQVVLTMTNMDPVDTATYYCAHYSGSYSYYYYGLDVWGQGTTVTVSS
HCDR1:
(SEQ ID NO: 133)
GFSLSTSGVG
HCDR2:
(SEQ ID NO: 134)
IYWNDHK
HCDR3:
(SEQ ID NO: 135)
AHYSGSYSYYYYGLDV
LCVR (V_{L}) Nucleotide Sequence
(SEQ ID NO: 136)
GACATCCAGATGACCCAGTCTCCATCTTCCGTGTCTGCATCTGTAGGAGACAGAGTCACCATCACTTGTCGGGCGAG
TCAGGGTATTGCCAGCTGGTTAGCCTGGTATCAGCAGAAACCAGGGAAAGCCCCTGAGCTCCTGATCTATGCTGCAT
CCAGTTTGCAAGGTGGGGTCCCATCAAGGTTCAGCGGCAGTGGATCTGGGACAGATTTCACTCTCACCATCAGCAGC
CTGCAGCCTGAAGATTTTGCAATTTACTATTGTCAACAGGCTAACTATTTCCCGTGGACGTTCGGCCAAGGGACCAA
GGTGGAAATCAAA
LCVR (V_{L}) Amino Acid Sequence
(SEQ ID NO: 137)
DIQMTQSPSSVSASVGDRVTITCRASQGIASWLAWYQQKPGKAPELLIYAASSLOGGVPSRFSGSGSGTDFTLTISS
LQPEDFAIYYCQQANYFPWTFGQGTKVEIK
LCDR1:
(SEQ ID NO: 138)
QGIASW
LCDR2:
(SEQ ID NO: 139)
AAS
LCDR3:
(SEQ ID NO: 140)
QQANYFPWT
12801B
HCVR (V_{H}) Nucleotide Sequence
(SEQ ID NO: 141)
GAGGTGCAGCTGTTGGAGTCTGGGGGAGCCTTGGTACAGCCTGGGGGGTCCCTGAGACTCTCCTGTGCAGCCTCTGG
ATTCACCTTTACCTCCTATGCCATGCACTGGGTCCGCCAGGCTCCAGGGAAGGGTCTGGAGTGGGTCTCATCTATTA
GAGGTAGTGGTGGTGGCACATACTCCGCAGACTCCGTGAAGGGCCGGTTCACCATCTCCAGAGACAATTCCAGGGAC
ACTCTATATCTGCAAATGAACAGTGTGAGAGCCGAGGACACGGCCGTTTATTACTGTGCGAGGTCCCATGACTACGG
TGCCTTCGACTTCTTTGACTACTGGGGCCAGGGAACCCTGGTCACCGTCTCCTCA
HCVR (V_{H}) Amino Acid Sequence
(SEQ ID NO: 142)
EVQLLESGGALVQPGGSLRLSCAASGFTFTSYAMHWVRQAPGKGLEWVSSIRGSGGGTYSADSVKGRFTISRDNSRD
TLYLQMNSVRAEDTAVYYCARSHDYGAFDFFDYWGQGTLVTVSS
or
(SEQ ID NO: 475)
EVQLLESGGALVQPGGSLRLSCAASGFTFTSYAMHWVRQAPGKGLEWVSSIRGSGGGTYSADSVKGRFTISRDNSRD
TLYLQMNSVRAEDTAVYYCARSHDYGAFDFFDYWGQGTTVTVSS
HCDR1:
(SEQ ID NO: 143)
GFTFTSYA
HCDR2:
(SEQ ID NO: 144)
IRGSGGGT
HCDR3:
(SEQ ID NO: 145)
ARSHDYGAFDFFDY
LCVR (V_{L}) Nucleotide Sequence
(SEQ ID NO: 146)
GACATCCAGATGACCCAGTCTCCATCCTCCCTGTCTGCATCTGTAGGAGACAGAGTCACCATCACTTGCCGGGCAAG
TCAGGGCATTAGAACTGATTTAGGCTGGTATCAGCAGAAACCAGGGAAAGCCCCTAAGCGCCTGATCTATGCTGCAT
CCAGTTTGCAAAGTGGGGTCCCATCAAGGTTCAGCGGCAGTGGATCTGGGACAGAATTCACTCTCACAATCAGCAGC
CTGCGGCCTGAAGATTTTGCAACTTTTTACTGTCTACAGTATAATAGTTACCCGCTCACTTTCGGCGGAGGGACCAA
GGTGGAGATCAAA
LCVR (V_{L}) Amino Acid Sequence
(SEQ ID NO: 147)
DIQMTQSPSSLSASVGDRVTITCRASQGIRTDLGWYQQKPGKAPKRLIYAASSLQSGVPSRFSGSGSGTEFTLTISS
LRPEDFATFYCLQYNSYPLTFGGGTKVEIK
or
(SEQ ID NO: 476)
DIQMTQSPSSLSASVGDRVTITCRASQGIRTDLGWYQQKPGKAPKRLIYAASSLQSGVPSRFSGSGSGTEFTLTISS
LRPEDFATFYCLQYNSYPLTFGGGTKVDIK
LCDR1:
(SEQ ID NO: 148)
QGIRTD
LCDR2:
(SEQ ID NO: 149)
AAS
LCDR3:
(SEQ ID NO: 150)
LQYNSYPLT
12802B (REGN16820 anti-hTfR scFv:hGAA)
HCVR (V_{H}) Nucleotide Sequence
(SEQ ID NO: 151)
CAGGTGCAGCTGGTGGAGTCTGGGGGAGGCTTGGTCAAGCCTGGAGGGTCCCTGAGACTCTCCTGTGCAGCCTCTGG
ATTCACCTTCAGTGACTACTTCATGAGCTGGATCCGCCAGGCTCCAGGGAAGGGGCTGGAGTGGGTTTCATACATTA
GTAGTACTGGTAGTACCATAAATTATGCAGACTCTGTGAAGGGCCGATTCACCATCTCCAGGGACAATGTCAAGAAT
TCACTGTATCTGCAAATGACCAGCCTGAGAGTCGAGGACACGGCCGTGTATTACTGTACGAGAGATAACTGGAACTA
TGAATACTGGGGCCAGGGAACCCTGGTCACCGTCTCCTCA
HCVR (V_{H}) Amino Acid Sequence
(SEQ ID NO: 152)
QVQLVESGGGLVKPGGSLRLSCAASGFTFSDYFMSWIRQAPGKGLEWVSYISSTGSTINYADSVKGRFTISRDNVKN
SLYLQMTSLRVEDTAVYYCTRDNWNYEYWGQGTLVTVSS
HCDR1:
(SEQ ID NO: 153)
GFTFSDYF
HCDR2:
(SEQ ID NO: 154)
ISSTGSTI
HCDR3:
(SEQ ID NO: 155)
TRDNWNYEY
LCVR (V_{L}) Nucleotide Sequence
(SEQ ID NO: 156)
GAAATAGTGATGACGCAGTCTCCAGCCACCCTGTCTGTGTCTCCAGGGGAAAGAGCCACCCTCTCCTGCAGGGCCAG
TCAGAGTGTTAGCATCAACTTAGCCTGGTACCAGCAGAAACCTGGCCAGGCTCCCAGGCTCCTCATCTTTGTTGCAT
CCACCAGGGCCACTGGTATCCCAGCCAGGTTCAGTGGCAGTGGGTCTGGGACAGAGTTCACTCTCACCATCAGCAGC
CTGCAGTCTGAAGATTTTGCAACTTATTACTGTCAGCAGTATGATATCTGGCCGTACACTTTTGGCCAGGGGACCAA
GCTGGAGATCAAA
LCVR (V_{L}) Amino Acid Sequence
(SEQ ID NO: 157)
EIVMTQSPATLSVSPGERATLSCRASQSVSINLAWYQQKPGQAPRLLIFVASTRATGIPARFSGSGSGTEFTLTISS
LOSEDFATYYCQQYDIWPYTFGQGTKLEIK
LCDR1:
(SEQ ID NO: 158)
QSVSIN
LCDR2:
(SEQ ID NO: 159)
VAS
LCDR3:
(SEQ ID NO: 160)
QQYDIWPYT
12808B
HCVR (V_{H}) Nucleotide Sequence
(SEQ ID NO: 161)
CAGCTGCAGCTGCAGGAGTCGGGCCCAGGACTGGTGAAGCCTTCGGAGACCCTGTCCCTCACCTGCACTGTGTCTGG
TGAATCCATCAGCAGTAATACTTACTACTGGGGCTGGATCCGCCAGCCCCCAGGGAAGGGGCTGGAATGGATTGGGA
GTATCGATTATAGTGGGACCACCAATTATAACCCGTCCCTCAAGAGTCGAGTCACCATATCCGTAGACACGTCCAGG
AATCACTTCTCCCTGAGGCTGAGGTCTGTGACCGCCGCAGACACGGCTGTGTATTACTGTGCGAGAGAGTGGGGAAA
CTACGGCTACTATTACGGTATGGACGTTTGGGGCCAAGGGACCACGGTCACCGTCTCCTCA
HCVR (V_{H}) Amino Acid Sequence
(SEQ ID NO: 162)
QLQLQESGPGLVKPSETLSLTCTVSGESISSNTYYWGWIRQPPGKGLEWIGSIDYSGTTNYNPSLKSRVTISVDTSR
NHFSLRLRSVTAADTAVYYCAREWGNYGYYYGMDVWGQGTTVTVSS
or
(SEQ ID NO: 477)
QVQLVESGPGLVKPSETLSLTCTVSGESISSNTYYWGWIRQPPGKGLEWIGSIDYSGTTNYNPSLKSRVTISVDTSR
NHFSLRLRSVTAADTAVYYCAREWGNYGYYYGMDVWGQGTTVTVSS
HCDR1:
(SEQ ID NO: 163)
GESISSNTYY
HCDR2:
(SEQ ID NO: 164)
IDYSGTT
HCDR3:
(SEQ ID NO: 165)
AREWGNYGYYYGMDV
LCVR (V_{L}) Nucleotide Sequence
(SEQ ID NO: 166)
GACATCCAGATGACCCAGTCTCCATCCTCCCTGTCTGCATCTGTAGGAGACAGAGTCACCATCAATTGCCGGGCAAG
TCAGGGCATTAGAAATGATTTAGGCTGGTATCAGCAGAAACCAGGGAAAGCCCCTAAGCGCCTGATCTATGCTGCAT
CCAGTTTGCAAAGTGGGGTCCCATTAAGGTTCAGTGGCAGTGGATCTGGGACAGAATTCACTCTCACAATCAACAAC
CTGCAGCCTGAAGATTTTGCAACTTATTACTGTCTATCGCATAATAGTTACCCGTGGACGTTCGGCCAAGGGACCAA
GGTGGAAATCAAA
LCVR (V_{L}) Amino Acid Sequence
(SEQ ID NO: 167)
DIQMTQSPSSLSASVGDRVTINCRASQGIRNDLGWYQQKPGKAPKRLIYAASSLQSGVPLRFSGSGSGTEFTLTINN
LQPEDFATYYCLSHNSYPWTFGQGTKVEIK
LCDR1:
(SEQ ID NO: 168)
QGIRND
LCDR2:
(SEQ ID NO: 169)
AAS
LCDR3:
(SEQ ID NO: 170)
LSHNSYPWT
12812B (REGN16821 anti-hTfR scFv:hGAA)
HCVR (V_{H}) Nucleotide Sequence
(SEQ ID NO: 171)
CAGGTGCAGCTGGTGCAGTCTGGGGCTGAGGTGAAGAAGCCTGGGTCCTCGGTGAGGGTCTCCTGCAAGGCTTCTAG
AGGCACCTTCAGCAGCTATGCTATCAGCTGGGTGCGACAGGCCCCTGGACAAGGCCTTGAGTGGATGGGAGGGATCA
TCCCCATCTTTGGTACAGCAAACTACGCACAGAAGTTCCTGGCCAGAGTCACGATTACCGCGGACGAATCCACGAGC
ACAGCCTACATGGAGCTGAGCAGCCTGAGATCTGAGGACACGGCCGTGTATTACTGTGCGAGAGAGAAGGGGTGGAA
CTACTTTGACTACTGGGGCCAGGGAACCCTGGTCACCGTCTCCTCA
HCVR (V_{H}) Amino Acid Sequence
(SEQ ID NO: 172)
QVQLVQSGAEVKKPGSSVRVSCKASRGTFSSYAISWVRQAPGQGLEWMGGIIPIFGTANYAQKFLARVTITADESTS
TAYMELSSLRSEDTAVYYCAREKGWNYFDYWGQGTLVTVSS
HCDR1:
(SEQ ID NO: 173)
RGTFSSYA
HCDR2:
(SEQ ID NO: 174)
IIPIFGTA
HCDR3:
(SEQ ID NO: 175)
AREKGWNYFDY
LCVR (V_{L}) Nucleotide Sequence
(SEQ ID NO: 176)
GACATCCAGATGACCCAGTCTCCACCTTCCGTGTCTGCATCTGTAGGAGACAGAGTCACCATCACTTGTCGGGCGAG
TCAGGGTATTAGCAGCTGGTTAGCCTGGTATCAGCAGAAACCAGGGAAAGCCCCTAAACTCCTGATCTATGCTGCAT
CCAGTTTGCAAAGTGGGGTCCCATCAAGGTTCAGCGGCAGTGGATCTGGGACAGATTTCACTCTCACCATCAGCAGC
CTGCAGCCTGAAGATTTTGCAACTTACTATTGTCAACAGGCTAACAGTTTCCCTCGGACGTTCGGCCAAGGGACCAA
GGTGGAAATCAAA
LCVR (V_{L}) Amino Acid Sequence
(SEQ ID NO: 177)
DIQMTQSPPSVSASVGDRVTITCRASQGISSWLAWYQQKPGKAPKLLIYAASSLQSGVPSRFSGSGSGTDFTLTISS
LQPEDFATYYCQQANSFPRTFGQGTKVEIK
LCDR1:
(SEQ ID NO: 178)
QGISSW
LCDR2:
(SEQ ID NO: 179)
AAS
LCDR3:
(SEQ ID NO: 180)
QQANSFPRT
12816B
HCVR (V_{H}) Nucleotide Sequence
(SEQ ID NO: 181)
CAGGTGCAGCTGGTGGAGTCTGGGGGAGGCTTGGTCAAGCCTGGAGGGTCCCTGAGACTCTCCTGTGCAGCCTCTGG
ATTCACCTTCAGTGACTACTACATGAACTGGATCCGCCAGGCTCCAGGGAAGGGGCTGGAGTGGGTTTCATACATTA
GTAGTAGTGGGACTACCATATACTACGCAGACTCTGTGAAGGGCCGATTCACCATCTCCAGGGACAACGCCAAGAAA
TCACTGTATCTGGAGATGAACAGCCTCAGAGCCGAGGACACGGCCGTGTACTACTGTGCGAGAGAGGGGTACGGTAA
TGACTACTATTACTACGGTATAGACGTCTGGGGCCAAGGGACCACGGTCACCGTCTCCTCA
HCVR (V_{H}) Amino Acid Sequence
(SEQ ID NO: 182)
QVQLVESGGGLVKPGGSLRLSCAASGFTFSDYYMNWIRQAPGKGLEWVSYISSSGTTIYYADSVKGRFTISRDNAKK
SLYLEMNSLRAEDTAVYYCAREGYGNDYYYYGIDVWGQGTTVTVSS
or
(SEQ ID NO: 478)
EVQLVESGGGLVKPGGSLRLSCAASGFTFSDYYMNWIRQAPGKGLEWVSYISSSGTTIYYADSVKGRFTISRDNAKK
SLYLEMNSLRAEDTAVYYCAREGYGNDYYYYGIDVWGQGTTVTVSS
HCDR1:
(SEQ ID NO: 183)
GFTFSDYY
HCDR2:
(SEQ ID NO: 184)
ISSSGTTI
HCDR3:
(SEQ ID NO: 185)
AREGYGNDYYYYGIDV
LCVR (V_{L}) Nucleotide Sequence
(SEQ ID NO: 186)
GATATTGTGATGACTCAGTCTCCACTCTCCCTGCCCGTCACCCCTGGAGAGCCGGCCTCCATCTCCTGCAGGTCTAG
TCAGAGCCTCCTGCATGGTAATGGATACAACTATTTGACTTGGTACCTGCAGAAGCCAGGGCAGTCTCCACAGCTCC
TGATCTATTTGGGTTCTAATCGGGCCTCCGGGGTCCCTGACAGGTTCAGTGGCAGTGGATCAGGCACAGATTTTACA
CTGAAAATAAGCAGAGTGGAGGCTGAGGATGTTGGGGTTTATTACTGCATGCAAGCTCTACAAACTCCGTACACTTT
TGGCCAGGGGACCAAGCTGGAGATCAAA
LCVR (V_{L}) Amino Acid Sequence
(SEQ ID NO: 187)
DIVMTQSPLSLPVTPGEPASISCRSSQSLLHGNGYNYLTWYLQKPGQSPQLLIYLGSNRASGVPDRESGSGSGTDFT
LKISRVEAEDVGVYYCMQALQTPYTFGQGTKLEIK
or
(SEQ ID NO: 479)
DIQLTQSPLSLPVTPGEPASISCRSSQSLLHGNGYNYLTWYLQKPGQSPQLLIYLGSNRASGVPDRESGSGSGTDFT
LKISRVEAEDVGVYYCMQALQTPYTFGQGTKVEIK
LCDR1:
(SEQ ID NO: 188)
QSLLHGNGYNY
LCDR2:
(SEQ ID NO: 189)
LGS
LCDR3:
(SEQ ID NO: 190)
MQALQTPYT
12833B
HCVR (V_{H}) Nucleotide Sequence
(SEQ ID NO: 191)
CAGGTGCAGCTGGTGGAGTCTGGGGGAGGCGTGGTCCAGCCTGGGAGGTCCCTGAGACTCTCCTGTGCAGCCTCTGG
ATTCACCTTCAGTAGCTTTGGCATGCACTGGGTCCGCCAGGCTCCAGGCAAGGGGCTGGAGTGGGTGATATTTATAT
CATATGATGGAAGTGATAAATACTATGCAGACTCCGTGAAGGGCCGATTCGCCATCTCCAGAGACAGTTCCAAGAAC
ACGCTATATCTGCAAATGAACAGCCTGAGAGCTGAGGACACGGCTGTGTATTACTGTGCGAAAGAAAACGGTATTTT
GACTGATTCCTACGGTATGGACGTCTGGGGCCAAGGGACCACGGTCACCGTCTCCTCA
HCVR (V_{H}) Amino Acid Sequence
(SEQ ID NO: 192)
QVQLVESGGGVVQPGRSLRLSCAASGFTFSSFGMHWVRQAPGKGLEWVIFISYDGSDKYYADSVKGRFAISRDSSKN
TLYLQMNSLRAEDTAVYYCAKENGILTDSYGMDVWGQGTTVTVSS
or
(SEQ ID NO: 480)
EVQLVESGGGVVQPGRSLRLSCAASGFTFSSFGMHWVRQAPGKGLEWVIFISYDGSDKYYADSVKGRFAISRDSSKN
TLYLQMNSLRAEDTAVYYCAKENGILTDSYGMDVWGQGTTVTVSS
HCDR1:
(SEQ ID NO: 193)
GFTFSSFG
HCDR2:
(SEQ ID NO: 194)
ISYDGSDK
HCDR3:
(SEQ ID NO: 195)
AKENGILTDSYGMDV
LCVR (V_{L}) Nucleotide Sequence
(SEQ ID NO: 196)
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
LCVR (V_{L}) Amino Acid Sequence
(SEQ ID NO: 197)
DIQMTQSPSSLSASVGDRVTITCRASQSISSYLNWYQQKPGKAPKLLIYAASSLQSGVPSRESGSGSGTDFTLTISS
LQPEDFATYYCQQSYSTPPITFGQGTRLEIK
LCDR1:
(SEQ ID NO: 198)
QSISSY
LCDR2:
(SEQ ID NO: 199)
AAS
LCDR3:
(SEQ ID NO: 200)
QQSYSTPPIT
12834B
HCVR (V_{H}) Nucleotide Sequence
(SEQ ID NO: 201)
CAGGTTCAGCTGGTGCAGTCTGGAGCTGAGGTGAAGAAGCCTGGGGCCTCTGTGAAGGTCTCCTGCAAGGCTTCTGG
TTACACCTTTACCAGCTATGGTATCAGCTGGGTGCGACAGGCCCCTGGACAAGGGCTTGAGTGGATGGGATGGATCA
GTGTTTACCATGGTAACACAAACTATGCACAGAAGTTCCAGGGCAGAGTCACCATGACCACAGACACATCCACGAGC
ACAGCCTACATGGAGCTGAGGAGCCTGAGATCTGACGACACGGCCGTGTATTACTGTGCGAGAGAGGGGTATTACGA
TTTTTGGAGTGGTTATTACCCTTTTGACTACTGGGGCCAGGGAACCCTGGTCACCGTCTCCTCA
HCVR (V_{H}) Amino Acid Sequence
(SEQ ID NO: 202)
QVQLVQSGAEVKKPGASVKVSCKASGYTFTSYGISWVRQAPGQGLEWMGWISVYHGNTNYAQKFQGRVTMTTDTSTS
TAYMELRSLRSDDTAVYYCAREGYYDFWSGYYPFDYWGQGTLVTVSS
or
(SEQ ID NO: 481)
EVQLVESGAEVKKPGASVKVSCKASGYTFTSYGISWVRQAPGQGLEWMGWISVYHGNTNYAQKFQGRVTMTTDTSTS
TAYMELRSLRSDDTAVYYCAREGYYDFWSGYYPFDYWGQGTTVTVSS
HCDR1:
(SEQ ID NO: 203)
GYTFTSYG
HCDR2:
(SEQ ID NO: 204)
ISVYHGNT
HCDR3:
(SEQ ID NO: 205)
AREGYYDFWSGYYPFDY
LCVR (V_{L}) Nucleotide Sequence
(SEQ ID NO: 206)
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
LCVR (V_{L}) Amino Acid Sequence
(SEQ ID NO: 207)
DIQMTQSPSSLSASVGDRVTITCRASQSISSYLNWYQQKPGKAPKLLIYAASSLQSGVPSRESGSGSGTDFTLTISS
LQPEDFATYYCQQSYSTPPITFGQGTRLEIK
LCDR1:
(SEQ ID NO: 208)
QSISSY
LCDR2:
(SEQ ID NO: 209)
AAS
LCDR3:
(SEQ ID NO: 210)
QQSYSTPPIT
12835B
HCVR (V_{H}) Nucleotide Sequence
(SEQ ID NO: 211)
GAGGTGCAGCTGGTGGAGTCTGGGGGAGGCTTGATACAACCTGGAGGGTCCCTGAGACTCTCCTGTGAAGCCTCTGG
ATTCACCTTCAGAAATTATGAAATGAATTGGGTCCGCCAGGCTCCAGGGAAGGGGCTGGAGTGGGTTTCATATATTA
GTAGTAGTGGTAATATGAAAGACTACGCAGAGTCTGTGAAGGGCCGATTCACCATCTCCAGAGACAATGTCAAGAAT
TCACTGCAGCTGCAAATGAACAGCCTGAGAGTCGAGGACACGGCTGTTTATTACTGTGCGAGAGACGAGTTTCCTTA
CGGAATGGACGTCTGGGGCCAAGGGACCACGGTCACCGTCTCCTCA
HCVR (V_{H}) Amino Acid Sequence
(SEQ ID NO: 212)
EVQLVESGGGLIQPGGSLRLSCEASGFTFRNYEMNWVRQAPGKGLEWVSYISSSGNMKDYAESVKGRFTISRDNVKN
SLQLQMNSLRVEDTAVYYCARDEFPYGMDVWGQGTTVTVSS
HCDR1:
(SEQ ID NO: 213)
GFTFRNYE
HCDR2:
(SEQ ID NO: 214)
ISSSGNMK
HCDR3:
(SEQ ID NO: 215)
ARDEFPYGMDV
LCVR (V_{L}) Nucleotide Sequence
(SEQ ID NO: 216)
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
LCVR (V_{L}) Amino Acid Sequence
(SEQ ID NO: 217)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIK
LCDR1:
(SEQ ID NO: 218)
QSISSY
LCDR2:
(SEQ ID NO: 219)
AAS
LCDR3:
(SEQ ID NO: 220)
QQSYSTPPIT
12847B (REGN17083 anti-hTfR Fab; REGN17077 anti-hTfR scFv;
REGN16826 anti-hTfR scFv:hGAA)
HCVR (V_{H}) Nucleotide Sequence
(SEQ ID NO: 221)
GAAGTGCAGCTGGTGGAGTCTGGGGGAGGCTTGGTTCAGCCTGGCAGGTCCCTGAGACTCTCCTGTGCAGCCTCTGG
ATTCACCTTTGATGATTATGCCATGAACTGGGTCCGGCAAGCTCCAGGGAAGGGCCTGGAGTGGGTCTCAGGTATTA
GTTGGAGTAGTGGTAGCATGGACTATGCGGACTCTGTGAAGGGCCGATTCACCATCTCCAGAGACAACGCCAAAAAC
TCCCTGTATCTGCAAATGAACAGTCTGAGAACTGAGGACACGGCCTTATATTACTGTGCAAAAGCTAGGGAAGTTGG
AGACTACTACGGTATGGACGTCTGGGGCCAAGGGACCACGGTCACCGTCTCCTCA
HCVR (V_{H}) Amino Acid Sequence
(SEQ ID NO: 222)
EVQLVESGGGLVQPGRSLRLSCAASGFTFDDYAMNWVRQAPGKGLEWVSGISWSSGSMDYADSVKGRFTISRDNAKN
SLYLQMNSLRTEDTALYYCAKAREVGDYYGMDVWGQGTTVTVSS
HCDR1:
(SEQ ID NO: 223)
GFTEDDYA
HCDR2:
(SEQ ID NO: 224)
ISWSSGSM
HCDR3:
(SEQ ID NO: 225)
AKAREVGDYYGMDV
LCVR (V_{L}) Nucleotide Sequence
(SEQ ID NO: 226)
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
LCVR (V_{L}) Amino Acid Sequence
(SEQ ID NO: 227)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIK
LCDR1:
(SEQ ID NO: 228)
QSISSY
LCDR2:
(SEQ ID NO: 229)
AAS
LCDR3:
(SEQ ID NO: 230)
QQSYSTPPIT
12848B (REGN16827 anti-hTfR scFv:hGAA)
HCVR (V_{H}) Nucleotide Sequence
(SEQ ID NO: 231)
GAAGTGCAGCTGGTGGAGTCTGGGGGAGGCTTGGTACAGCCTGGCAGGTCCCTGACACTCTCCTGTGCAGCCTCTGG
ATTCACCTTTGATAATTTTGGCATGCACTGGGTCCGGCAAGGTCCAGGGAAGGGCCTGGAATGGGTCTCAGGTCTTA
CTTGGAATAGTGGTGTCATAGGCTATGCGGACTCTGTGAAGGGCCGATTCACCATCTCCAGAGACAACGCCAAGAAC
TCCCTGTATCTGCAAATGAACAGTCTGAGACCTGAGGACACGGCCTTATATTACTGTGCAAAAGATATACGGAATTA
CGGCCCCTTTGACTACTGGGGCCAGGGAACCCTGGTCACCGTCTCCTCA
HCVR (V_{H}) Amino Acid Sequence
(SEQ ID NO: 232)
EVQLVESGGGLVQPGRSLTLSCAASGFTFDNFGMHWVRQGPGKGLEWVSGLTWNSGVIGYADSVKGRFTISRDNAKN
SLYLQMNSLRPEDTALYYCAKDIRNYGPFDYWGQGTLVTVSS
HCDR1:
(SEQ ID NO: 233)
GFTFDNFG
HCDR2:
(SEQ ID NO: 234)
LTWNSGVI
HCDR3:
(SEQ ID NO: 235)
AKDIRNYGPFDY
LCVR (V_{L}) Nucleotide Sequence
(SEQ ID NO: 236)
GAAATTGTGTTGACGCAGTCTCCAGGCACCCTGTCTTTGTCTCCAGGGGAAAGAGCCACCCTCTCCTGCAGGGCCAG
TCAGAGTGTTAGCAGCAGCTACTTAGCCTGGTACCAGCAGAAACCTGGCCAGGCTCCCAGGCTCCTCATCTATGGTG
CATCCAGCAGGGCCACTGGCATCCCAGACAGGTTCAGTGGCAGTGGGTCTGGGACAGACTTCACTCTCACCATCAGC
AGACTGGAGCCTGAAGATTTTGCAGTGTATTACTGTCAGCAGTATGGTAGCTCACCTTGGACGTTCGGCCAAGGGAC
CAAGGTGGAAATCAAA
LCVR (V_{L}) Amino Acid Sequence
(SEQ ID NO: 237)
EIVLTQSPGTLSLSPGERATLSCRASQSVSSSYLAWYQQKPGQAPRLLIYGASSRATGIPDRESGSGSGTDFTLTIS
RLEPEDFAVYYCQQYGSSPWTFGQGTKVEIK
LCDR1:
(SEQ ID NO: 238)
QSVSSSY
LCDR2:
(SEQ ID NO: 239)
GAS
LCDR3:
(SEQ ID NO: 240)
QQYGSSPWT
12843B (REGN17075 anti-hTfR scFv; REGN16824 anti-hTfR scFv:hGAA;
REGN17081 anti-hTfR Fab)
HCVR (V_{H}) Nucleotide Sequence
(SEQ ID NO: 241)
GAGGTGCAGCTGGTGGAGTCTGGGGGAGGCTTAGTACAGCCTGGAGGGTCCCTAAGACTCTCCTGTGCAGCCTCTGG
ATTCACCTTCAATATTTTTGAAATGAACTGGGTCCGCCAGGCTCCAGGGAAGGGGCTGGAGTGGATTTCCTACATTA
GTAGTCGTGGAACTACCACATACTACGCAGACTCTGTGAGGGGCCGATTCACCATCTCCAGAGACAACGCCAAGAAC
TCACTGTATCTGCAAATGAACAGCCTGAGAGCCGAGGACACGGCTGTTTATTACTGTGCGAGAGATTATGAAGCAAC
AATCCCTTTTGACTTCTGGGGCCAGGGAACCCTGGTCACCGTCTCCTCA
HCVR (V_{H}) Amino Acid Sequence
(SEQ ID NO: 242)
EVQLVESGGGLVQPGGSLRLSCAASGFTENIFEMNWVRQAPGKGLEWISYISSRGTTTYYADSVRGRFTISRDNAKN
SLYLQMNSLRAEDTAVYYCARDYEATIPFDFWGQGTLVTVSS
HCDR1:
(SEQ ID NO: 243)
GFTFNIFE
HCDR2:
(SEQ ID NO: 244)
ISSRGTTT
HCDR3:
(SEQ ID NO: 245)
ARDYEATIPFDF
LCVR (V_{L}) Nucleotide Sequence
(SEQ ID NO: 246)
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
LCVR (V_{L}) Amino Acid Sequence
(SEQ ID NO: 247)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIK
LCDR1:
(SEQ ID NO: 248)
QSISSY
LCDR2:
(SEQ ID NO: 249)
AAS
LCDR3:
(SEQ ID NO: 250)
QQSYSTPPIT
12844B
HCVR (V_{H}) Nucleotide Sequence
(SEQ ID NO: 251)
GAGGTGCAGCTGGTGGAGTCTGGGGGAAGTGTGGTACGGCCTGGGGGGTCCCTGAGACTCTCCTGTGAAGCCTCTGG
ATTCACCTTTGATGATTATGGCATGAGCTGGGTCCGCCAAGATCCAGGGAAGGGGCTGGAGTGGGTCTCTGGTATTA
ATTGGAATGGTGATAGAACAAATTATGCAGACTCTGTGAAGGGCCGATTCATCATTTCCAGAGACAACGCCAAGAAC
TCTGTGTATCTACAAATGAACAGTCTGAGAGCGGAGGACTCGGCCTTGTATCACTGTGCGAGAGATCAGGGACTCGG
AGTGGCAGCTACCCTTGACTACTGGGGCCAGGGAACCCTGGTCACCGTCTCCTCA
HCVR (V_{H}) Amino Acid Sequence
(SEQ ID NO: 252)
EVQLVESGGSVVRPGGSLRLSCEASGFTFDDYGMSWVRQDPGKGLEWVSGINWNGDRTNYADSVKGRFIISRDNAKN
SVYLQMNSLRAEDSALYHCARDQGLGVAATLDYWGQGTLVTVSS
or
(SEQ ID NO: 482)
EVQLVESGGSVVRPGGSLRLSCEASGFTFDDYGMSWVRQDPGKGLEWVSGINWNGDRTNYADSVKGRFIISRDNAKN
SVYLQMNSLRAEDSALYHCARDQGLGVAATLDYWGQGTMVTVSS
HCDR1:
(SEQ ID NO: 253)
GFTEDDYG
HCDR2:
(SEQ ID NO: 254)
INWNGDRT
HCDR3:
(SEQ ID NO: 255)
ARDQGLGVAATLDY
LCVR (V_{L}) Nucleotide Sequence
(SEQ ID NO: 256)
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
LCVR (V_{L}) Amino Acid Sequence
(SEQ ID NO: 257)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIK
LCDR1:
(SEQ ID NO: 258)
QSISSY
LCDR2:
(SEQ ID NO: 259)
AAS
LCDR3:
(SEQ ID NO: 260)
QQSYSTPPIT
12845B (REGN17082 Fab; REGN17076 scFv; REGN16825 anti-hTfR scFv:hGAA)
HCVR (V_{H}) Nucleotide Sequence
(SEQ ID NO: 261)
GAGGTGCAGCTGGTGGAGTCTGGGGGAGGCTTGGTACAGCCTGGAGGGTCCCTGAGACTCTCCTGTGCAGCCTCTGG
ATTCACCGTCAGTAATTATGAAATGAACTGGGTCCGCCAGGCTCCAGGGAAGGGGCTGGAGTGGGTTTCATACATTA
GTAGTAGTACCAGTAACATATACTACGCAGACTCTGTGAAGGGCCGATTCACCATCTCCAGAGACAACGCCGAGAAC
TCACTGTATCTGCAGATGAACAGCCTGAGAGTCGAGGACACGGCTGTTTATTACTGTGTGAGAGATGGGATTGTAGT
AGTTCCAGTTGGTCGTGGATACTACTATTACGGTTTGGACGTCTGGGGCCAAGGGACCACGGTCACCGTCTCCTCA
HCVR (V_{H}) Amino Acid Sequence
(SEQ ID NO: 262)
EVQLVESGGGLVQPGGSLRLSCAASGFTVSNYEMNWVRQAPGKGLEWVSYISSSTSNIYYADSVKGRFTISRDNAEN
SLYLQMNSLRVEDTAVYYCVRDGIVVVPVGRGYYYYGLDVWGQGTTVTVSS
HCDR1:
(SEQ ID NO: 263)
GFTVSNYE
HCDR2:
(SEQ ID NO: 264)
ISSSTSNI
HCDR3:
(SEQ ID NO: 265)
VRDGIVVVPVGRGYYYYGLDV
LCVR (V_{L}) Nucleotide Sequence
(SEQ ID NO: 266)
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
LCVR (V_{L}) Amino Acid Sequence
(SEQ ID NO: 267)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIK
LCDR1:
(SEQ ID NO: 268)
QSISSY
LCDR2:
(SEQ ID NO: 269)
AAS
LCDR3:
(SEQ ID NO: 270)
QQSYSTPPIT
12839B (REGN17080 Fab; REGN17074 scFv; REGN16822 anti-hTfR scFv:hGAA)
HCVR (V_{H}) Nucleotide Sequence
(SEQ ID NO: 271)
CAGGTGCAGCTGGTGGAGTCTGGGGGAGGCGTGGTCCAGCCTGGAAGGTCCCTGAGACTCTCCTGCGCAGCCTCTGG
ATTCCCCTTTAGTAATTATGTCATGTATTGGGTCCGCCAGGCTCCAGGCAAGGGGCTGGAGTGGGTGGCTCTTATTT
TTTTTGACGGAAAGAAAAACTATCATGCAGACTCCGTGAAGGGCCGATTCACCATAACCAGAGACAATTCCAAAAAT
ATGTTATATCTGCAAATGAACAGCCTGAGACCTGAGGACGCGGCTGTGTATTACTGTGCGAAAATCCATTGTCCTAA
TGGTGTATGTTACAAGGGGTATTACGGAATGGACGTCTGGGGCCAAGGGACCACGGTCACCGTCTCCTCA
HCVR (V_{H}) Amino Acid Sequence
(SEQ ID NO: 272)
QVQLVESGGGVVQPGRSLRLSCAASGFPFSNYVMYWVRQAPGKGLEWVALIFFDGKKNYHADSVKGRFTITRDNSKN
MLYLQMNSLRPEDAAVYYCAKIHCPNGVCYKGYYGMDVWGQGTTVTVSS
HCDR1:
(SEQ ID NO: 273)
GFPFSNYV
HCDR2:
(SEQ ID NO: 274)
IFFDGKKN
HCDR3:
(SEQ ID NO: 275)
AKIHCPNGVCYKGYYGMDV
LCVR (V_{L}) Nucleotide Sequence
(SEQ ID NO: 276)
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
LCVR (V_{L}) Amino Acid Sequence
(SEQ ID NO: 277)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIK
LCDR1:
(SEQ ID NO: 278)
QSISSY
LCDR2:
(SEQ ID NO: 279)
AAS
LCDR3:
(SEQ ID NO: 280)
QQSYSTPPIT
12841B (REGN16823 anti-hTfR scFv:hGAA)
HCVR (V_{H}) Nucleotide Sequence
(SEQ ID NO: 281)
GAGGTGCAGCTGGTGGAGTCTGGGGGAGGCTTGGTCCAGCCTGGGGGGTCCCTAAGACTCTCCTGTGCAGCCTCTGG
ATTCACCTTTAGTAACTATTGGATGAACTGGGTCCGCCAGGCTCCAGGGAAGGGACTGGAGTGGGTGGCCAATATAA
AAGAAGATGGAGGTAAGAAATTGTATGTGGACTCTGTGAAGGGCCGATTCACCATCTCCAGAGACAACGCCAAGAAC
TCACTGTTTCTGCAAATGAACAGCCTGAGAGCCGAGGACACGGCTGTGTATTATTGTGCGAGAGAAGATACAACTTT
GGTTGTGGACTACTACTACTACGGTATGGACGTCTGGGGCCAAGGGACCACGGTCACCGTCTCCTCA
HCVR (V_{H}) Amino Acid Sequence
(SEQ ID NO: 282)
EVQLVESGGGLVQPGGSLRLSCAASGFTFSNYWMNWVRQAPGKGLEWVANIKEDGGKKLYVDSVKGRFTISRDNAKN
SLFLQMNSLRAEDTAVYYCAREDTTLVVDYYYYGMDVWGQGTTVTVSS
HCDR1:
(SEQ ID NO: 283)
GFTFSNYW
HCDR2:
(SEQ ID NO: 284)
IKEDGGKK
HCDR3:
(SEQ ID NO: 285)
AREDTTLVVDYYYYGMDV
LCVR (V_{L}) Nucleotide Sequence
(SEQ ID NO: 286)
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
LCVR (V_{L}) Amino Acid Sequence
(SEQ ID NO: 287)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIK
LCDR1:
(SEQ ID NO: 288)
QSISSY
LCDR2:
(SEQ ID NO: 289)
AAS
LCDR3:
(SEQ ID NO: 290)
QQSYSTPPIT
12850B (REGN16828 anti-hTfR scFv:hGAA)
HCVR (V_{H}) Nucleotide Sequence
(SEQ ID NO: 291)
CAGGTCCAGCTGGTGCAGTCTGGGGCTGAGGTGAAGAAGCCTGGGTCCTCGGTGAAGGTCTCCTGCAAGGCTTCTGG
AGGCACCTTCAACACCTATGCTATCACCTGGGTGCGACAGGCCCCTGGACAAGGGCTTGAATGGATGGGGGGAATCA
TCCCTATCTCTGGCATAGCAGAGTACGCACAGAAGTTCCAGGGCAGAGTCACGATCACCACGGATGACTCCTCGACC
ACAGCCTACATGGAACTGAACAGTCTGAGATCTGAGGACACGGCCGTGTATTACTGTGCGAGCTGGAACTACGCACT
CTACTACTTCTACGGTATGGACGTCTGGGGCCGAGGGACCACGGTCACCGTCTCCTCA
HCVR (V_{H}) Amino Acid Sequence
(SEQ ID NO: 292)
QVQLVQSGAEVKKPGSSVKVSCKASGGTFNTYAITWVRQAPGOGLEWMGGIIPISGIAEYAQKFQGRVTITTDDSST
TAYMELNSLRSEDTAVYYCASWNYALYYFYGMDVWGRGTTVTVSS
HCDR1:
(SEQ ID NO: 293)
GGTENTYA
HCDR2:
(SEQ ID NO: 294)
IIPISGIA
HCDR3:
(SEQ ID NO: 295)
ASWNYALYYFYGMDV
LCVR (V_{L}) Nucleotide Sequence
(SEQ ID NO: 296)
GAAATTGTGTTGACGCAGTCTCCAGGCACCCTGTCTTTGTCTCCAGGGGAAAGAGCCACCCTCTCCTGCAGGGCCAG
TCAGAGTGTTAGCAGCAGCTACTTAGCCTGGTACCAGCAGAAACCTGGCCAGGCTCCCAGGCTCCTCATCTATGGTG
CATCCAGCAGGGCCACTGGCATCCCAGACAGGTTCAGTGGCAGTGGGTCTGGGACAGACTTCACTCTCACCATCAGC
AGACTGGAGCCTGAAGATTTTGCAGTGTATTACTGTCAGCAGTATGGTAGCTCACCTTGGACGTTCGGCCAAGGGAC
CAAGGTGGAAATCAAA
LCVR (V_{L}) Amino Acid Sequence
(SEQ ID NO: 297)
EIVLTQSPGTLSLSPGERATLSCRASQSVSSSYLAWYQQKPGQAPRLLIYGASSRATGIPDRESGSGSGTDFTLTIS
RLEPEDFAVYYCQQYGSSPWTFGQGTKVEIK
LCDR1:
(SEQ ID NO: 298)
QSVSSSY
LCDR2:
(SEQ ID NO: 299)
GAS
LCDR3:
(SEQ ID NO: 300)
QQYGSSPWT
69261
HCVR (V_{H}) Nucleotide Sequence
(SEQ ID NO: 301)
CAGGTGCAGCTGGTGGAGTCTGGGGGAGGCTTGGTCAAGCCTGGAGGGTCCCTGAGACTCTCCTGTGCAGCCTCTGG
ATTCACCTTCAGTGTCTATTACATGAACTGGATCCGCCAGGCTCCAGGGAAGGGCCTGGAGTGGGTTTCATACATTA
GTAGTAGTGGTAGTACCATATACTACGCAGACTCTGTGAAGGGCCGATTCACCATCTCCAGGGACAACGCCAAGAAC
TCACTGTATCTCCAAATGAACAGTCTGAGAGCCGAGGACACGGCCGTATATTACTGTGGGAGAGAAGGGTATAGTGG
GACTTATTCTTATTACGGTATGGACGTCTGGGGCCAAGGGACCACGGTCACCGTCTCCTCA
HCVR (V_{H}) Amino Acid Sequence
(SEQ ID NO: 302)
QVQLVESGGGLVKPGGSLRLSCAASGFTFSVYYMNWIRQAPGKGLEWVSYISSSGSTIYYADSVKGRFTISRDNAKN
SLYLQMNSLRAEDTAVYYCGREGYSGTYSYYGMDVWGQGTTVTVSS
or
(SEQ ID NO: 483)
EVQLVESGGGLVKPGGSLRLSCAASGFTFSVYYMNWIRQAPGKGLEWVSYISSSGSTIYYADSVKGRFTISRDNAKN
SLYLQMNSLRAEDTAVYYCGREGYSGTYSYYGMDVWGQGTTVTVSS
HCDR1:
(SEQ ID NO: 303)
GFTFSVYY
HCDR2:
(SEQ ID NO: 304)
ISSSGSTI
HCDR3:
(SEQ ID NO: 305)
GREGYSGTYSYYGMDV
LCVR (V_{L}) Nucleotide Sequence
(SEQ ID NO: 306)
GATATTGTGATGACTCAGTCTCCACTCTCCCTGCCCGTCACCCCTGGAGAGCCGGCCTCCATCTCCTGCAGGTCTAG
TCAGAGCCTCCTGCATAGTAATGGATACAACTATTTGGATTGGTACCTGCAGAAGCCAGGGCAGTCTCCACAGTTCC
TGATCTATTTGGGTTCTAATCGGGCCTCCGGGGTCCCTGACAGGTTCAGTGGCAGTGGATCAGGCACAGATTTTACA
CTGAAAATCAACAGAGTGGAGGCTGAGGATGTTGGGGTTTATTACTGCATGCAAGCTCTACAAACTCCGTACACTTT
TGGCCAGGGGACCAAGCTGGAGATCAAA
LCVR (V_{L}) Amino Acid Sequence
(SEQ ID NO: 307)
DIVMTQSPLSLPVTPGEPASISCRSSQSLLHSNGYNYLDWYLQKPGQSPQFLIYLGSNRASGVPDRESGSGSGTDFT
LKINRVEAEDVGVYYCMQALQTPYTFGQGTKLEIK
or
(SEQ ID NO: 488)
DIQLTQSPLSLPVTPGEPASISCRSSQSLLHSNGYNYLDWYLQKPGQSPQFLIYLGSNRASGVPDRFSGSGSGTDFT
LKINRVEAEDVGVYYCMQALQTPYTFGQGTKVEIK
LCDR1:
(SEQ ID NO: 308)
QSLLHSNGYNY
LCDR2:
(SEQ ID NO: 309)
LGS
LCDR3:
(SEQ ID NO: 310)
MQALQTPYT
69263
HCVR (V_{H}) Nucleotide Sequence
(SEQ ID NO: 311)
GAAGTGCAGCTGGTGGAGTCTGGGGGAGGGTTGGTACAGCCTGGCAGGTCCCTGAGACTCTCCTGTGCAGTCTCTGG
ATTCACCTTTGATGATTATGCCATGCACTGGGTCCGGCAAGCTCCAGGGAAGGGCCTGGAGTGGGTCTCAGGTATTA
GTTGGAATAGTGGTACCAGAGGATATGCGGACTCTGTGAAGGGCCGATTCACCATCTCCAGAGACAACGCCAAGAAC
TCCCTGTATCTGCAAATGAACAGTCTGAGAGGTGAGGACACGGCCTTGTATTACTGTGTAAAAGATATTACGATATC
CCCCAACTACTACGGTATGGACGTCTGGGGCCAAGGGACCACGGTCACCGTCTCCTCA
HCVR (V_{H}) Amino Acid Sequence
(SEQ ID NO: 312)
EVQLVESGGGLVQPGRSLRLSCAVSGFTFDDYAMHWVRQAPGKGLEWVSGISWNSGTRGYADSVKGRFTISRDNAKN
SLYLQMNSLRGEDTALYYCVKDITISPNYYGMDVWGQGTTVTVSS
HCDR1:
(SEQ ID NO: 313)
GFTEDDYA
HCDR2:
(SEQ ID NO: 314)
ISWNSGTR
HCDR3:
(SEQ ID NO: 315)
VKDITISPNYYGMDV
LCVR (V_{L}) Nucleotide Sequence
(SEQ ID NO: 316)
GACATCCAGATGACCCAGTCTCCATCCTCCCTGTCTGCATCTGTAGGAGACAGAGTCACCATCACTTGCCGGGCGAG
TCAGGACATTAGCCATTATTCAGCCTGGTATCAGCAGAAACCAGGGAAACTTCCTAACCTCCTGATCTATGCTGCAT
CCACTTTGCAATCAGGGGTCCCATCTCGGTTCAGTGGCAGTGGATCTGGGACAGATTTCTCTCTCACCACCAGCAGC
CTGCAGCCTGAAGATGTTGCAACTTATTACTGTCAAAAGTATAACAGTGTCCCTCTCACTTTCGGCGGAGGGACCAA
GGTGGAGATCAAA
LCVR (V_{L}) Amino Acid Sequence
(SEQ ID NO: 317)
DIQMTQSPSSLSASVGDRVTITCRASQDISHYSAWYQQKPGKLPNLLIYAASTLQSGVPSRFSGSGSGTDESLTTSS
LQPEDVATYYCQKYNSVPLTFGGGTKVEIK
or
(SEQ ID NO: 484)
DIQLTQSPSSLSASVGDRVTITCRASQDISHYSAWYQQKPGKLPNLLIYAASTLQSGVPSRFSGSGSGTDESLTTSS
LQPEDVATYYCQKYNSVPLTFGGRTKVEIK
LCDR1:
(SEQ ID NO: 318)
QDISHY
LCDR2:
(SEQ ID NO: 319)
AAS
LCDR3:
(SEQ ID NO: 320)
QKYNSVPLT

As discussed, an anti-hTfR:Payload scFv fusion protein (e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263) comprises an optional signal peptide, connected to an scFv (e.g., including a V_{L} and a V_{H} optionally connected by a linker), connected to an optional linker, connected to a payload such as GAA or variant thereof wherein, for example:
(I) the optional signal peptide is, for example, the signal peptide from Mus musculus Ror (e.g., consisting of the amino acids MHRPRRRGTRPPPLALLAALLLAARGADA (SEQ ID NO: 500));
(II) the scFv comprises:
(i) a heavy chain variable region that comprises the HCDR1, HCDR2 and HCDR3 of a HCVR comprising the amino acid sequence set forth in SEQ ID NO: 2; 462; 12; 463; 22; 464; 32; 42; 52; 467; 62; 492; 72; 470; 82; 92; 472; 102; 112; 473; 122; 132; 142; 475; 152; 162; 477; 172; 182; 478; 192; 480; 202; 481; 212; 222; 232; 242; 252; 482; 262; 272; 282; 292; 302; 483 or 312; and/or
(ii) a light chain variable region that comprises the LCDR1, LCDR2 and LCDR3 of a LCVR comprising the amino acid sequence set forth in SEQ ID NO: 7; 17; 27; 37; 465; 47; 466; 57; 468; 67; 469; 77; 471; 87; 97; 107; 117; 474; 127; 137; 147; 476; 157; 167; 177; 187; 479; 197; 207; 217; 227; 237; 247; 257; 267; 277; 287; 297; 307; 488; 317 or 484; or the scFv comprises:
(1) a HCVR comprising the HCDR1, HCDR2 and HCDR3 of a HCVR that comprises the amino acid sequence set forth in SEQ ID NO: 2 or 462 (or a variant thereof); and a LCVR comprising the LCDR1, LCDR2 and LCDR3 of a LCVR that comprises the amino acid sequence set forth in SEQ ID NO: 7 (or a variant thereof);
(2) a HCVR comprising the HCDR1, HCDR2 and HCDR3 of a HCVR that comprises the amino acid sequence set forth in SEQ ID NO: 12 or 463 (or a variant thereof); and a LCVR comprising the LCDR1, LCDR2 and LCDR3 of a LCVR that comprises the amino acid sequence set forth in SEQ ID NO: 17 (or a variant thereof);
(3) a HCVR comprising the HCDR1, HCDR2 and HCDR3 of a HCVR that comprises the amino acid sequence set forth in SEQ ID NO: 22 or 464 (or a variant thereof); and a LCVR comprising the LCDR1, LCDR2 and LCDR3 of a LCVR that comprises the amino acid sequence set forth in SEQ ID NO: 27 (or a variant thereof);
(4) a HCVR comprising the HCDR1, HCDR2 and HCDR3 of a HCVR that comprises the amino acid sequence set forth in SEQ ID NO: 32 (or a variant thereof); and a LCVR comprising the LCDR1, LCDR2 and LCDR3 of a LCVR that comprises the amino acid sequence set forth in SEQ ID NO: 37 or 465 (or a variant thereof);
(5) a HCVR comprising the HCDR1, HCDR2 and HCDR3 of a HCVR that comprises the amino acid sequence set forth in SEQ ID NO: 42 (or a variant thereof); and a LCVR comprising the LCDR1, LCDR2 and LCDR3 of a LCVR that comprises the amino acid sequence set forth in SEQ ID NO: 47 or 466 (or a variant thereof);
(6) a HCVR comprising the HCDR1, HCDR2 and HCDR3 of a HCVR that comprises the amino acid sequence set forth in SEQ ID NO: 52 or 467 (or a variant thereof); and a LCVR comprising the LCDR1, LCDR2 and LCDR3 of a LCVR that comprises the amino acid sequence set forth in SEQ ID NO: 57 or 468 (or a variant thereof);
(7) a HCVR comprising the HCDR1, HCDR2 and HCDR3 of a HCVR that comprises the amino acid sequence set forth in SEQ ID NO: 62 or 492 (or a variant thereof); and a LCVR comprising the LCDR1, LCDR2 and LCDR3 of a LCVR that comprises the amino acid sequence set forth in SEQ ID NO: 67 or 469 (or a variant thereof);
(8) a HCVR comprising the HCDR1, HCDR2 and HCDR3 of a HCVR that comprises the amino acid sequence set forth in SEQ ID NO: 72 or 470 (or a variant thereof); and a LCVR comprising the LCDR1, LCDR2 and LCDR3 of a LCVR that comprises the amino acid sequence set forth in SEQ ID NO: 77 or 471 (or a variant thereof);
(9) a HCVR comprising the HCDR1, HCDR2 and HCDR3 of a HCVR that comprises the amino acid sequence set forth in SEQ ID NO: 82 (or a variant thereof); and a LCVR comprising the LCDR1, LCDR2 and LCDR3 of a LCVR that comprises the amino acid sequence set forth in SEQ ID NO: 87 (or a variant thereof);
(10) a HCVR comprising the HCDR1, HCDR2 and HCDR3 of a HCVR that comprises the amino acid sequence set forth in SEQ ID NO: 92 or 472 (or a variant thereof); and a LCVR comprising the LCDR1, LCDR2 and LCDR3 of a LCVR that comprises the amino acid sequence set forth in SEQ ID NO: 97 (or a variant thereof);
(11) a HCVR comprising the HCDR1, HCDR2 and HCDR3 of a HCVR that comprises the amino acid sequence set forth in SEQ ID NO: 102 (or a variant thereof); and a LCVR comprising the LCDR1, LCDR2 and LCDR3 of a LCVR that comprises the amino acid sequence set forth in SEQ ID NO: 107 (or a variant thereof);
(12) a HCVR comprising the HCDR1, HCDR2 and HCDR3 of a HCVR that comprises the amino acid sequence set forth in SEQ ID NO: 112 or 473 (or a variant thereof); and a LCVR comprising the LCDR1, LCDR2 and LCDR3 of a LCVR that comprises the amino acid sequence set forth in SEQ ID NO: 117 or 474 (or a variant thereof);
(13) a HCVR comprising the HCDR1, HCDR2 and HCDR3 of a HCVR that comprises the amino acid sequence set forth in SEQ ID NO: 122 (or a variant thereof); and a LCVR comprising the LCDR1, LCDR2 and LCDR3 of a LCVR that comprises the amino acid sequence set forth in SEQ ID NO: 127 (or a variant thereof);
(14) a HCVR comprising the HCDR1, HCDR2 and HCDR3 of a HCVR that comprises the amino acid sequence set forth in SEQ ID NO: 132 (or a variant thereof); and a LCVR comprising the LCDR1, LCDR2 and LCDR3 of a LCVR that comprises the amino acid sequence set forth in SEQ ID NO: 137 (or a variant thereof);
(15) a HCVR comprising the HCDR1, HCDR2 and HCDR3 of a HCVR that comprises the amino acid sequence set forth in SEQ ID NO: 142 or 475 (or a variant thereof); and a LCVR comprising the LCDR1, LCDR2 and LCDR3 of a LCVR that comprises the amino acid sequence set forth in SEQ ID NO: 147 or 476 (or a variant thereof);
(16) a HCVR comprising the HCDR1, HCDR2 and HCDR3 of a HCVR that comprises the amino acid sequence set forth in SEQ ID NO: 152 (or a variant thereof); and a LCVR comprising the LCDR1, LCDR2 and LCDR3 of a LCVR that comprises the amino acid sequence set forth in SEQ ID NO: 157 (or a variant thereof);
(17) a HCVR comprising the HCDR1, HCDR2 and HCDR3 of a HCVR that comprises the amino acid sequence set forth in SEQ ID NO: 162 or 477 (or a variant thereof); and a LCVR comprising the LCDR1, LCDR2 and LCDR3 of a LCVR that comprises the amino acid sequence set forth in SEQ ID NO: 167 (or a variant thereof);
(18) a HCVR comprising the HCDR1, HCDR2 and HCDR3 of a HCVR that comprises the amino acid sequence set forth in SEQ ID NO: 172 (or a variant thereof); and a LCVR comprising the LCDR1, LCDR2 and LCDR3 of a LCVR that comprises the amino acid sequence set forth in SEQ ID NO: 177 (or a variant thereof);
(19) a HCVR comprising the HCDR1, HCDR2 and HCDR3 of a HCVR that comprises the amino acid sequence set forth in SEQ ID NO: 182 or 478 (or a variant thereof); and a LCVR comprising the LCDR1, LCDR2 and LCDR3 of a LCVR that comprises the amino acid sequence set forth in SEQ ID NO: 187 or 479 (or a variant thereof);
(20) a HCVR comprising the HCDR1, HCDR2 and HCDR3 of a HCVR that comprises the amino acid sequence set forth in SEQ ID NO: 192 or 480 (or a variant thereof); and a LCVR comprising the LCDR1, LCDR2 and LCDR3 of a LCVR that comprises the amino acid sequence set forth in SEQ ID NO: 197 (or a variant thereof);
(21) a HCVR comprising the HCDR1, HCDR2 and HCDR3 of a HCVR that comprises the amino acid sequence set forth in SEQ ID NO: 202 or 481 (or a variant thereof); and a LCVR comprising the LCDR1, LCDR2 and LCDR3 of a LCVR that comprises the amino acid sequence set forth in SEQ ID NO: 207 (or a variant thereof);
(22) a HCVR comprising the HCDR1, HCDR2 and HCDR3 of a HCVR that comprises the amino acid sequence set forth in SEQ ID NO: 212 (or a variant thereof); and a LCVR comprising the LCDR1, LCDR2 and LCDR3 of a LCVR that comprises the amino acid sequence set forth in SEQ ID NO: 217 (or a variant thereof);
(23) a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof);
(24) a HCVR comprising the HCDR1, HCDR2 and HCDR3 of a HCVR that comprises the amino acid sequence set forth in SEQ ID NO: 232 (or a variant thereof); and a LCVR comprising the LCDR1, LCDR2 and LCDR3 of a LCVR that comprises the amino acid sequence set forth in SEQ ID NO: 237 (or a variant thereof);
(25) a HCVR comprising the HCDR1, HCDR2 and HCDR3 of a HCVR that comprises the amino acid sequence set forth in SEQ ID NO: 242 (or a variant thereof); and a LCVR comprising the LCDR1, LCDR2 and LCDR3 of a LCVR that comprises the amino acid sequence set forth in SEQ ID NO: 247 (or a variant thereof);
(26) a HCVR comprising the HCDR1, HCDR2 and HCDR3 of a HCVR that comprises the amino acid sequence set forth in SEQ ID NO: 252 or 482 (or a variant thereof); and a LCVR comprising the LCDR1, LCDR2 and LCDR3 of a LCVR that comprises the amino acid sequence set forth in SEQ ID NO: 257 (or a variant thereof);
(27) a HCVR comprising the HCDR1, HCDR2 and HCDR3 of a HCVR that comprises the amino acid sequence set forth in SEQ ID NO: 262 (or a variant thereof); and a LCVR comprising the LCDR1, LCDR2 and LCDR3 of a LCVR that comprises the amino acid sequence set forth in SEQ ID NO: 267 (or a variant thereof);
(28) a HCVR comprising the HCDR1, HCDR2 and HCDR3 of a HCVR that comprises the amino acid sequence set forth in SEQ ID NO: 272 (or a variant thereof); and a LCVR comprising the LCDR1, LCDR2 and LCDR3 of a LCVR that comprises the amino acid sequence set forth in SEQ ID NO: 277 (or a variant thereof);
(29) a HCVR comprising the HCDR1, HCDR2 and HCDR3 of a HCVR that comprises the amino acid sequence set forth in SEQ ID NO: 282 (or a variant thereof); and a LCVR comprising the LCDR1, LCDR2 and LCDR3 of a LCVR that comprises the amino acid sequence set forth in SEQ ID NO: 287 (or a variant thereof);
(30) a HCVR comprising the HCDR1, HCDR2 and HCDR3 of a HCVR that comprises the amino acid sequence set forth in SEQ ID NO: 292 (or a variant thereof); and a LCVR comprising the LCDR1, LCDR2 and LCDR3 of a LCVR that comprises the amino acid sequence set forth in SEQ ID NO: 297 (or a variant thereof);
(31) a HCVR comprising the HCDR1, HCDR2 and HCDR3 of a HCVR that comprises the amino acid sequence set forth in SEQ ID NO: 302 or 483 (or a variant thereof); and a LCVR comprising the LCDR1, LCDR2 and LCDR3 of a LCVR that comprises the amino acid sequence set forth in SEQ ID NO: 307 or 488 (or a variant thereof);
(32) a HCVR comprising the HCDR1, HCDR2 and HCDR3 of a HCVR that comprises the amino acid sequence set forth in SEQ ID NO: 312 (or a variant thereof); and a LCVR comprising the LCDR1, LCDR2 and LCDR3 of a LCVR that comprises the amino acid sequence set forth in SEQ ID NO: 317 or 484 (or a variant thereof);
or the scFv comprises:
(a) a HCVR that comprises:
an HCDR1 comprising the amino acid sequence set forth in SEQ ID NO: 3 (or a variant thereof),
an HCDR2 comprising the amino acid sequence set forth in SEQ ID NO: 4 (or a variant thereof), and
an HCDR3 comprising the amino acid sequence set forth in SEQ ID NO: 5 (or a variant thereof); and
a LCVR that comprises:
an LCDR1 comprising the amino acid sequence set forth in SEQ ID NO: 8 (or a variant thereof),
an LCDR2 comprising the amino acid sequence set forth in SEQ ID NO: 9 (or a variant thereof), and
an LCDR3 comprising the amino acid sequence set forth in SEQ ID NO: 10 (or a variant thereof);
(b) a HCVR that comprises:
an HCDR1 comprising the amino acid sequence set forth in SEQ ID NO: 13 (or a variant thereof),
an HCDR2 comprising the amino acid sequence set forth in SEQ ID NO: 14 (or a variant thereof), and
an HCDR3 comprising the amino acid sequence set forth in SEQ ID NO: 15 (or a variant thereof); and
a LCVR that comprises:
an LCDR1 comprising the amino acid sequence set forth in SEQ ID NO: 18 (or a variant thereof),
an LCDR2 comprising the amino acid sequence set forth in SEQ ID NO: 19 (or a variant thereof), and
an LCDR3 comprising the amino acid sequence set forth in SEQ ID NO: 20 (or a variant thereof);
(c) a HCVR that comprises:
an HCDR1 comprising the amino acid sequence set forth in SEQ ID NO: 23 (or a variant thereof),
an HCDR2 comprising the amino acid sequence set forth in SEQ ID NO: 24 (or a variant thereof), and
an HCDR3 comprising the amino acid sequence set forth in SEQ ID NO: 25 (or a variant thereof); and
a LCVR that comprises:
an LCDR1 comprising the amino acid sequence set forth in SEQ ID NO: 28 (or a variant thereof),
an LCDR2 comprising the amino acid sequence set forth in SEQ ID NO: 29 (or a variant thereof), and
an LCDR3 comprising the amino acid sequence set forth in SEQ ID NO: 30 (or a variant thereof);
(d) a HCVR that comprises:
an HCDR1 comprising the amino acid sequence set forth in SEQ ID NO: 33 (or a variant thereof),
an HCDR2 comprising the amino acid sequence set forth in SEQ ID NO: 34 (or a variant thereof), and
an HCDR3 comprising the amino acid sequence set forth in SEQ ID NO: 35 (or a variant thereof); and
a LCVR that comprises:
an LCDR1 comprising the amino acid sequence set forth in SEQ ID NO: 38 (or a variant thereof),
an LCDR2 comprising the amino acid sequence set forth in SEQ ID NO: 39 (or a variant thereof), and
an LCDR3 comprising the amino acid sequence set forth in SEQ ID NO: 40 (or a variant thereof);
(e) a HCVR that comprises:
an HCDR1 comprising the amino acid sequence set forth in SEQ ID NO: 43 (or a variant thereof),
an HCDR2 comprising the amino acid sequence set forth in SEQ ID NO: 44 (or a variant thereof), and
an HCDR3 comprising the amino acid sequence set forth in SEQ ID NO: 45 (or a variant thereof); and
a LCVR that comprises:
an LCDR1 comprising the amino acid sequence set forth in SEQ ID NO: 48 (or a variant thereof),
an LCDR2 comprising the amino acid sequence set forth in SEQ ID NO: 49 (or a variant thereof), and
an LCDR3 comprising the amino acid sequence set forth in SEQ ID NO: 50 (or a variant thereof);
(f) a HCVR that comprises:
an HCDR1 comprising the amino acid sequence set forth in SEQ ID NO: 53 (or a variant thereof),
an HCDR2 comprising the amino acid sequence set forth in SEQ ID NO: 54 (or a variant thereof), and
an HCDR3 comprising the amino acid sequence set forth in SEQ ID NO: 55 (or a variant thereof); and
a LCVR that comprises:
an LCDR1 comprising the amino acid sequence set forth in SEQ ID NO: 58 (or a variant thereof),
an LCDR2 comprising the amino acid sequence set forth in SEQ ID NO: 59 (or a variant thereof), and
an LCDR3 comprising the amino acid sequence set forth in SEQ ID NO: 60 (or a variant thereof);
(g) a HCVR that comprises:
an HCDR1 comprising the amino acid sequence set forth in SEQ ID NO: 63 (or a variant thereof),
an HCDR2 comprising the amino acid sequence set forth in SEQ ID NO: 64 (or a variant thereof), and
an HCDR3 comprising the amino acid sequence set forth in SEQ ID NO: 65 (or a variant thereof); and
a LCVR that comprises:
an LCDR1 comprising the amino acid sequence set forth in SEQ ID NO: 68 (or a variant thereof),
an LCDR2 comprising the amino acid sequence set forth in SEQ ID NO: 69 (or a variant thereof), and
an LCDR3 comprising the amino acid sequence set forth in SEQ ID NO: 70 (or a variant thereof);
(h) a HCVR that comprises:
an HCDR1 comprising the amino acid sequence set forth in SEQ ID NO: 73 (or a variant thereof),
an HCDR2 comprising the amino acid sequence set forth in SEQ ID NO: 74 (or a variant thereof), and
an HCDR3 comprising the amino acid sequence set forth in SEQ ID NO: 75 (or a variant thereof); and
a LCVR that comprises:
an LCDR1 comprising the amino acid sequence set forth in SEQ ID NO: 78 (or a variant thereof),
an LCDR2 comprising the amino acid sequence set forth in SEQ ID NO: 79 (or a variant thereof), and
an LCDR3 comprising the amino acid sequence set forth in SEQ ID NO: 80 (or a variant thereof);
(i) a HCVR that comprises:
an HCDR1 comprising the amino acid sequence set forth in SEQ ID NO: 83 (or a variant thereof),
an HCDR2 comprising the amino acid sequence set forth in SEQ ID NO: 84 (or a variant thereof), and
an HCDR3 comprising the amino acid sequence set forth in SEQ ID NO: 85 (or a variant thereof); and
a LCVR that comprises:
an LCDR1 comprising the amino acid sequence set forth in SEQ ID NO: 88 (or a variant thereof),
an LCDR2 comprising the amino acid sequence set forth in SEQ ID NO: 89 (or a variant thereof), and
an LCDR3 comprising the amino acid sequence set forth in SEQ ID NO: 90 (or a variant thereof);
(j) a HCVR that comprises:
an HCDR1 comprising the amino acid sequence set forth in SEQ ID NO: 93 (or a variant thereof),
an HCDR2 comprising the amino acid sequence set forth in SEQ ID NO: 94 (or a variant thereof), and
an HCDR3 comprising the amino acid sequence set forth in SEQ ID NO: 95 (or a variant thereof); and
a LCVR that comprises:
an LCDR1 comprising the amino acid sequence set forth in SEQ ID NO: 98 (or a variant thereof),
an LCDR2 comprising the amino acid sequence set forth in SEQ ID NO: 99 (or a variant thereof), and
an LCDR3 comprising the amino acid sequence set forth in SEQ ID NO: 100 (or a variant thereof);
(k) a HCVR that comprises:
an HCDR1 comprising the amino acid sequence set forth in SEQ ID NO: 103 (or a variant thereof),
an HCDR2 comprising the amino acid sequence set forth in SEQ ID NO: 104 (or a variant thereof), and
an HCDR3 comprising the amino acid sequence set forth in SEQ ID NO: 105 (or a variant thereof); and
a LCVR that comprises:
an LCDR1 comprising the amino acid sequence set forth in SEQ ID NO: 108 (or a variant thereof),
an LCDR2 comprising the amino acid sequence set forth in SEQ ID NO: 109 (or a variant thereof), and
an LCDR3 comprising the amino acid sequence set forth in SEQ ID NO: 110 (or a variant thereof);
(l) a HCVR that comprises:
an HCDR1 comprising the amino acid sequence set forth in SEQ ID NO: 113 (or a variant thereof),
an HCDR2 comprising the amino acid sequence set forth in SEQ ID NO: 114 (or a variant thereof), and
an HCDR3 comprising the amino acid sequence set forth in SEQ ID NO: 115 (or a variant thereof); and
a LCVR that comprises:
an LCDR1 comprising the amino acid sequence set forth in SEQ ID NO: 118 (or a variant thereof),
an LCDR2 comprising the amino acid sequence set forth in SEQ ID NO: 119 (or a variant thereof), and
an LCDR3 comprising the amino acid sequence set forth in SEQ ID NO: 120 (or a variant thereof);
(m) a HCVR that comprises:
an HCDR1 comprising the amino acid sequence set forth in SEQ ID NO: 123 (or a variant thereof),
an HCDR2 comprising the amino acid sequence set forth in SEQ ID NO: 124 (or a variant thereof), and
an HCDR3 comprising the amino acid sequence set forth in SEQ ID NO: 125 (or a variant thereof); and
a LCVR that comprises:
an LCDR1 comprising the amino acid sequence set forth in SEQ ID NO: 128 (or a variant thereof),
an LCDR2 comprising the amino acid sequence set forth in SEQ ID NO: 129 (or a variant thereof), and
an LCDR3 comprising the amino acid sequence set forth in SEQ ID NO: 130 (or a variant thereof);
(n) a HCVR that comprises:
an HCDR1 comprising the amino acid sequence set forth in SEQ ID NO: 133 (or a variant thereof),
an HCDR2 comprising the amino acid sequence set forth in SEQ ID NO: 134 (or a variant thereof), and
an HCDR3 comprising the amino acid sequence set forth in SEQ ID NO: 135 (or a variant thereof); and
a LCVR that comprises:
an LCDR1 comprising the amino acid sequence set forth in SEQ ID NO: 138 (or a variant thereof),
an LCDR2 comprising the amino acid sequence set forth in SEQ ID NO: 139 (or a variant thereof), and
an LCDR3 comprising the amino acid sequence set forth in SEQ ID NO: 140 (or a variant thereof);
(o) a HCVR that comprises:
an HCDR1 comprising the amino acid sequence set forth in SEQ ID NO: 143 (or a variant thereof),
an HCDR2 comprising the amino acid sequence set forth in SEQ ID NO: 144 (or a variant thereof), and
an HCDR3 comprising the amino acid sequence set forth in SEQ ID NO: 145 (or a variant thereof); and
a LCVR that comprises:
an LCDR1 comprising the amino acid sequence set forth in SEQ ID NO: 148 (or a variant thereof),
an LCDR2 comprising the amino acid sequence set forth in SEQ ID NO: 149 (or a variant thereof), and
an LCDR3 comprising the amino acid sequence set forth in SEQ ID NO: 150 (or a variant thereof);
(p) a HCVR that comprises:
an HCDR1 comprising the amino acid sequence set forth in SEQ ID NO: 153 (or a variant thereof),
an HCDR2 comprising the amino acid sequence set forth in SEQ ID NO: 154 (or a variant thereof), and
an HCDR3 comprising the amino acid sequence set forth in SEQ ID NO: 155 (or a variant thereof); and
a LCVR that comprises:
an LCDR1 comprising the amino acid sequence set forth in SEQ ID NO: 158 (or a variant thereof),
an LCDR2 comprising the amino acid sequence set forth in SEQ ID NO: 159 (or a variant thereof), and
an LCDR3 comprising the amino acid sequence set forth in SEQ ID NO: 160 (or a variant thereof);
(q) a HCVR that comprises:
an HCDR1 comprising the amino acid sequence set forth in SEQ ID NO: 163 (or a variant thereof),
an HCDR2 comprising the amino acid sequence set forth in SEQ ID NO: 164 (or a variant thereof), and
an HCDR3 comprising the amino acid sequence set forth in SEQ ID NO: 165 (or a variant thereof); and
a LCVR that comprises:
an LCDR1 comprising the amino acid sequence set forth in SEQ ID NO: 168 (or a variant thereof),
an LCDR2 comprising the amino acid sequence set forth in SEQ ID NO: 169 (or a variant thereof), and
an LCDR3 comprising the amino acid sequence set forth in SEQ ID NO: 170 (or a variant thereof);
(r) a HCVR that comprises:
an HCDR1 comprising the amino acid sequence set forth in SEQ ID NO: 173 (or a variant thereof),
an HCDR2 comprising the amino acid sequence set forth in SEQ ID NO: 174 (or a variant thereof), and
an HCDR3 comprising the amino acid sequence set forth in SEQ ID NO: 175 (or a variant thereof); and
a LCVR that comprises:
an LCDR1 comprising the amino acid sequence set forth in SEQ ID NO: 178 (or a variant thereof),
an LCDR2 comprising the amino acid sequence set forth in SEQ ID NO: 179 (or a variant thereof), and
an LCDR3 comprising the amino acid sequence set forth in SEQ ID NO: 180 (or a variant thereof);
(s) a HCVR that comprises:
an HCDR1 comprising the amino acid sequence set forth in SEQ ID NO: 183 (or a variant thereof),
an HCDR2 comprising the amino acid sequence set forth in SEQ ID NO: 184 (or a variant thereof), and
an HCDR3 comprising the amino acid sequence set forth in SEQ ID NO: 185 (or a variant thereof); and a LCVR that comprises:
an LCDR1 comprising the amino acid sequence set forth in SEQ ID NO: 188 (or a variant thereof),
an LCDR2 comprising the amino acid sequence set forth in SEQ ID NO: 189 (or a variant thereof), and
an LCDR3 comprising the amino acid sequence set forth in SEQ ID NO: 190 (or a variant thereof);
(t) a HCVR that comprises:
an HCDR1 comprising the amino acid sequence set forth in SEQ ID NO: 193 (or a variant thereof),
an HCDR2 comprising the amino acid sequence set forth in SEQ ID NO: 194 (or a variant thereof), and
an HCDR3 comprising the amino acid sequence set forth in SEQ ID NO: 195 (or a variant thereof); and
a LCVR that comprises:
an LCDR1 comprising the amino acid sequence set forth in SEQ ID NO: 198 (or a variant thereof),
an LCDR2 comprising the amino acid sequence set forth in SEQ ID NO: 199 (or a variant thereof), and
an LCDR3 comprising the amino acid sequence set forth in SEQ ID NO: 200 (or a variant thereof);
(u) a HCVR that comprises:
an HCDR1 comprising the amino acid sequence set forth in SEQ ID NO: 203 (or a variant thereof),
an HCDR2 comprising the amino acid sequence set forth in SEQ ID NO: 204 (or a variant thereof), and
an HCDR3 comprising the amino acid sequence set forth in SEQ ID NO: 205 (or a variant thereof); and
a LCVR that comprises:
an LCDR1 comprising the amino acid sequence set forth in SEQ ID NO: 208 (or a variant thereof),
an LCDR2 comprising the amino acid sequence set forth in SEQ ID NO: 209 (or a variant thereof), and
an LCDR3 comprising the amino acid sequence set forth in SEQ ID NO: 210 (or a variant thereof);
(v) a HCVR that comprises:
an HCDR1 comprising the amino acid sequence set forth in SEQ ID NO: 213 (or a variant thereof),
an HCDR2 comprising the amino acid sequence set forth in SEQ ID NO: 214 (or a variant thereof), and
an HCDR3 comprising the amino acid sequence set forth in SEQ ID NO: 215 (or a variant thereof); and
a LCVR that comprises:
an LCDR1 comprising the amino acid sequence set forth in SEQ ID NO: 218 (or a variant thereof),
an LCDR2 comprising the amino acid sequence set forth in SEQ ID NO: 219 (or a variant thereof), and
an LCDR3 comprising the amino acid sequence set forth in SEQ ID NO: 220 (or a variant thereof);
(w) a HCVR that comprises:
an HCDR1 comprising the amino acid sequence set forth in SEQ ID NO: 223 (or a variant thereof),
an HCDR2 comprising the amino acid sequence set forth in SEQ ID NO: 224 (or a variant thereof), and
an HCDR3 comprising the amino acid sequence set forth in SEQ ID NO: 225 (or a variant thereof); and
a LCVR that comprises:
an LCDR1 comprising the amino acid sequence set forth in SEQ ID NO: 228 (or a variant thereof),
an LCDR2 comprising the amino acid sequence set forth in SEQ ID NO: 229 (or a variant thereof), and
an LCDR3 comprising the amino acid sequence set forth in SEQ ID NO: 230 (or a variant thereof);
(x) a HCVR that comprises:
an HCDR1 comprising the amino acid sequence set forth in SEQ ID NO: 233 (or a variant thereof),
an HCDR2 comprising the amino acid sequence set forth in SEQ ID NO: 234 (or a variant thereof), and
an HCDR3 comprising the amino acid sequence set forth in SEQ ID NO: 235 (or a variant thereof); and
a LCVR that comprises:
an LCDR1 comprising the amino acid sequence set forth in SEQ ID NO: 238 (or a variant thereof),
an LCDR2 comprising the amino acid sequence set forth in SEQ ID NO: 239 (or a variant thereof), and
an LCDR3 comprising the amino acid sequence set forth in SEQ ID NO: 240 (or a variant thereof);
(y) a HCVR that comprises:
an HCDR1 comprising the amino acid sequence set forth in SEQ ID NO: 243 (or a variant thereof),
an HCDR2 comprising the amino acid sequence set forth in SEQ ID NO: 244 (or a variant thereof), and
an HCDR3 comprising the amino acid sequence set forth in SEQ ID NO: 245 (or a variant thereof); and
a LCVR that comprises:
an LCDR1 comprising the amino acid sequence set forth in SEQ ID NO: 248 (or a variant thereof),
an LCDR2 comprising the amino acid sequence set forth in SEQ ID NO: 249 (or a variant thereof), and
an LCDR3 comprising the amino acid sequence set forth in SEQ ID NO: 250 (or a variant thereof);
(z) a HCVR that comprises:
an HCDR1 comprising the amino acid sequence set forth in SEQ ID NO: 253 (or a variant thereof),
an HCDR2 comprising the amino acid sequence set forth in SEQ ID NO: 254 (or a variant thereof), and
an HCDR3 comprising the amino acid sequence set forth in SEQ ID NO: 255 (or a variant thereof); and
a LCVR that comprises:
an LCDR1 comprising the amino acid sequence set forth in SEQ ID NO: 258 (or a variant thereof),
an LCDR2 comprising the amino acid sequence set forth in SEQ ID NO: 259 (or a variant thereof), and
an LCDR3 comprising the amino acid sequence set forth in SEQ ID NO: 260 (or a variant thereof);
(aa) a HCVR that comprises:
an HCDR1 comprising the amino acid sequence set forth in SEQ ID NO: 263 (or a variant thereof),
an HCDR2 comprising the amino acid sequence set forth in SEQ ID NO: 264 (or a variant thereof), and
an HCDR3 comprising the amino acid sequence set forth in SEQ ID NO: 265 (or a variant thereof); and
a LCVR that comprises:
an LCDR1 comprising the amino acid sequence set forth in SEQ ID NO: 268 (or a variant thereof),
an LCDR2 comprising the amino acid sequence set forth in SEQ ID NO: 269 (or a variant thereof), and
an LCDR3 comprising the amino acid sequence set forth in SEQ ID NO: 270 (or a variant thereof);
(ab) a HCVR that comprises:
an HCDR1 comprising the amino acid sequence set forth in SEQ ID NO: 273 (or a variant thereof),
an HCDR2 comprising the amino acid sequence set forth in SEQ ID NO: 274 (or a variant thereof), and
an HCDR3 comprising the amino acid sequence set forth in SEQ ID NO: 275 (or a variant thereof); and
a LCVR that comprises:
an LCDR1 comprising the amino acid sequence set forth in SEQ ID NO: 278 (or a variant thereof),
an LCDR2 comprising the amino acid sequence set forth in SEQ ID NO: 279 (or a variant thereof), and
an LCDR3 comprising the amino acid sequence set forth in SEQ ID NO: 280 (or a variant thereof);
(ac) a HCVR that comprises:
an HCDR1 comprising the amino acid sequence set forth in SEQ ID NO: 283 (or a variant thereof),
an HCDR2 comprising the amino acid sequence set forth in SEQ ID NO: 284 (or a variant thereof), and
an HCDR3 comprising the amino acid sequence set forth in SEQ ID NO: 285 (or a variant thereof); and
a LCVR that comprises:
an LCDR1 comprising the amino acid sequence set forth in SEQ ID NO: 288 (or a variant thereof),
an LCDR2 comprising the amino acid sequence set forth in SEQ ID NO: 289 (or a variant thereof), and
an LCDR3 comprising the amino acid sequence set forth in SEQ ID NO: 290 (or a variant thereof);
(ad) a HCVR that comprises:
an HCDR1 comprising the amino acid sequence set forth in SEQ ID NO: 293 (or a variant thereof),
an HCDR2 comprising the amino acid sequence set forth in SEQ ID NO: 294 (or a variant thereof), and
an HCDR3 comprising the amino acid sequence set forth in SEQ ID NO: 295 (or a variant thereof); and
a LCVR that comprises:
an LCDR1 comprising the amino acid sequence set forth in SEQ ID NO: 298 (or a variant thereof),
an LCDR2 comprising the amino acid sequence set forth in SEQ ID NO: 299 (or a variant thereof), and
an LCDR3 comprising the amino acid sequence set forth in SEQ ID NO: 300 (or a variant thereof);
(ae) a HCVR that comprises:
an HCDR1 comprising the amino acid sequence set forth in SEQ ID NO: 303 (or a variant thereof),
an HCDR2 comprising the amino acid sequence set forth in SEQ ID NO: 304 (or a variant thereof), and
an HCDR3 comprising the amino acid sequence set forth in SEQ ID NO: 305 (or a variant thereof); and
a LCVR that comprises:
an LCDR1 comprising the amino acid sequence set forth in SEQ ID NO: 308 (or a variant thereof),
an LCDR2 comprising the amino acid sequence set forth in SEQ ID NO: 309 (or a variant thereof), and
an LCDR3 comprising the amino acid sequence set forth in SEQ ID NO: 310 (or a variant thereof);
and/or
(af) a HCVR that comprises:
an HCDR1 comprising the amino acid sequence set forth in SEQ ID NO: 313 (or a variant thereof),
an HCDR2 comprising the amino acid sequence set forth in SEQ ID NO: 314 (or a variant thereof), and
an HCDR3 comprising the amino acid sequence set forth in SEQ ID NO: 315 (or a variant thereof); and
a LCVR that comprises:
an LCDR1 comprising the amino acid sequence set forth in SEQ ID NO: 318 (or a variant thereof),
an LCDR2 comprising the amino acid sequence set forth in SEQ ID NO: 319 (or a variant thereof), and
an LCDR3 comprising the amino acid sequence set forth in SEQ ID NO: 320 (or a variant thereof);
or the scFv comprises:
(i) a HCVR that comprises the amino acid sequence set forth in SEQ ID NO: 2 or 462 (or a variant thereof); and a LCVR that comprises the amino acid sequence set forth in SEQ ID NO: 7 (or a variant thereof);
(ii) a HCVR that comprises the amino acid sequence set forth in SEQ ID NO: 12 or 463 (or a variant thereof); and a LCVR that comprises the amino acid sequence set forth in SEQ ID NO: 17 (or a variant thereof);
(iii) a HCVR that comprises the amino acid sequence set forth in SEQ ID NO: 22 or 464 (or a variant thereof); and a LCVR that comprises the amino acid sequence set forth in SEQ ID NO: 27 (or a variant thereof);
(iv) a HCVR that comprises the amino acid sequence set forth in SEQ ID NO: 32 (or a variant thereof); and a LCVR that comprises the amino acid sequence set forth in SEQ ID NO: 37 or 465 (or a variant thereof);
(v) a HCVR that comprises the amino acid sequence set forth in SEQ ID NO: 42 (or a variant thereof); and a LCVR that comprises the amino acid sequence set forth in SEQ ID NO: 47 or 466 (or a variant thereof);
(vi) a HCVR that comprises the amino acid sequence set forth in SEQ ID NO: 52 or 467 (or a variant thereof); and a LCVR that comprises the amino acid sequence set forth in SEQ ID NO: 57 or 468 (or a variant thereof);
(vii) a HCVR that comprises the amino acid sequence set forth in SEQ ID NO: 62 or 492 (or a variant thereof); and a LCVR that comprises the amino acid sequence set forth in SEQ ID NO: 67 or 469 (or a variant thereof);
(viii) a HCVR that comprises the amino acid sequence set forth in SEQ ID NO: 72 or 470 (or a variant thereof); and a LCVR that comprises the amino acid sequence set forth in SEQ ID NO: 77 or 471 (or a variant thereof);
(ix) a HCVR that comprises the amino acid sequence set forth in SEQ ID NO: 82 (or a variant thereof); and a LCVR that comprises the amino acid sequence set forth in SEQ ID NO: 87 (or a variant thereof);
(x) a HCVR that comprises the amino acid sequence set forth in SEQ ID NO: 92 or 472 (or a variant thereof); and a LCVR that comprises the amino acid sequence set forth in SEQ ID NO: 97 (or a variant thereof);
(xi) a HCVR that comprises the amino acid sequence set forth in SEQ ID NO: 102 (or a variant thereof); and a LCVR that comprises the amino acid sequence set forth in SEQ ID NO: 107 (or a variant thereof);
(xii) a HCVR that comprises the amino acid sequence set forth in SEQ ID NO: 112 or 473 (or a variant thereof); and a LCVR that comprises the amino acid sequence set forth in SEQ ID NO: 117 or 474 (or a variant thereof);
(xiii) a HCVR that comprises the amino acid sequence set forth in SEQ ID NO: 122 (or a variant thereof); and a LCVR that comprises the amino acid sequence set forth in SEQ ID NO: 127 (or a variant thereof);
(xiv) a HCVR that comprises the amino acid sequence set forth in SEQ ID NO: 132 (or a variant thereof); and a LCVR that comprises the amino acid sequence set forth in SEQ ID NO: 137 (or a variant thereof);
(xv) a HCVR that comprises the amino acid sequence set forth in SEQ ID NO: 142 or 475 (or a variant thereof); and a LCVR that comprises the amino acid sequence set forth in SEQ ID NO: 147 or 476 (or a variant thereof);
(xvi) a HCVR that comprises the amino acid sequence set forth in SEQ ID NO: 152 (or a variant thereof); and a LCVR that comprises the amino acid sequence set forth in SEQ ID NO: 157 (or a variant thereof);
(xvii) a HCVR that comprises the amino acid sequence set forth in SEQ ID NO: 162 or 477 (or a variant thereof); and a LCVR that comprises the amino acid sequence set forth in SEQ ID NO: 167 (or a variant thereof);
(xviii) a HCVR that comprises the amino acid sequence set forth in SEQ ID NO: 172 (or a variant thereof); and a LCVR that comprises the amino acid sequence set forth in SEQ ID NO: 177 (or a variant thereof);
(xix) a HCVR that comprises the amino acid sequence set forth in SEQ ID NO: 182 or 478 (or a variant thereof); and a LCVR that comprises the amino acid sequence set forth in SEQ ID NO: 187 or 479 (or a variant thereof);
(xx) a HCVR that comprises the amino acid sequence set forth in SEQ ID NO: 192 or 480 (or a variant thereof); and a LCVR that comprises the amino acid sequence set forth in SEQ ID NO: 197 (or a variant thereof);
(xxi) a HCVR that comprises the amino acid sequence set forth in SEQ ID NO: 202 or 481 (or a variant thereof); and a LCVR that comprises the amino acid sequence set forth in SEQ ID NO: 207 (or a variant thereof);
(xxii) a HCVR that comprises the amino acid sequence set forth in SEQ ID NO: 212 (or a variant thereof); and a LCVR that comprises the amino acid sequence set forth in SEQ ID NO: 217 (or a variant thereof);
(xxiii) a HCVR that comprises the amino acid sequence set forth in SEQ ID NO: 222 (or a variant thereof); and a LCVR that comprises the amino acid sequence set forth in SEQ ID NO: 227 (or a variant thereof);
(xxiv) a HCVR that comprises the amino acid sequence set forth in SEQ ID NO: 232 (or a variant thereof); and a LCVR that comprises the amino acid sequence set forth in SEQ ID NO: 237 (or a variant thereof);
(xxv) a HCVR that comprises the amino acid sequence set forth in SEQ ID NO: 242 (or a variant thereof); and a LCVR that comprises the amino acid sequence set forth in SEQ ID NO: 247 (or a variant thereof);
(xxvi) a HCVR that comprises the amino acid sequence set forth in SEQ ID NO: 252 or 482 (or a variant thereof); and a LCVR that comprises the amino acid sequence set forth in SEQ ID NO: 257 (or a variant thereof);
(xxvii) a HCVR that comprises the amino acid sequence set forth in SEQ ID NO: 262 (or a variant thereof); and a LCVR that comprises the amino acid sequence set forth in SEQ ID NO: 267 (or a variant thereof);
(xxviii) a HCVR that comprises the amino acid sequence set forth in SEQ ID NO: 272 (or a variant thereof); and a LCVR that comprises the amino acid sequence set forth in SEQ ID NO: 277 (or a variant thereof);
(xxix) a HCVR that comprises the amino acid sequence set forth in SEQ ID NO: 282 (or a variant thereof); and a LCVR that comprises the amino acid sequence set forth in SEQ ID NO: 287 (or a variant thereof);
(xxx) a HCVR that comprises the amino acid sequence set forth in SEQ ID NO: 292 (or a variant thereof); and a LCVR that comprises the amino acid sequence set forth in SEQ ID NO: 297 (or a variant thereof);
(xxxi) a HCVR that comprises the amino acid sequence set forth in SEQ ID NO: 302 or 483 (or a variant thereof); and a LCVR that comprises the amino acid sequence set forth in SEQ ID NO: 307 or 488 (or a variant thereof); and/or
(xxxii) a HCVR that comprises the amino acid sequence set forth in SEQ ID NO: 312 (or a variant thereof); and a LCVR that comprises the amino acid sequence set forth in SEQ ID NO: 317 or 484 (or a variant thereof);
e.g., wherein the HCVR and LCVR are in either orientation (HCVR-LCVR or LCVR-HCVR), optionally, wherein the HCVR and LCVR are linked by a linker, e.g., that comprises an amino acid sequence, e.g., about 10 amino acids in length, for example:
(Gly₄Ser)ₘ wherein m is 1, 2, 3, 4, 5, 6, 7, 8, 9 or 10 (Gly₄Ser=SEQ ID NO: 426);
(III) the optional linker comprises an amino acid sequence, e.g., about 10 amino acids in length, for example: (Gly₄Ser)ₘ wherein m is 1, 2, 3, 4, 5, 6, 7, 8, 9 or 10, e.g., 2 (Gly₄Ser=SEQ ID NO: 426); and,
(IV) payload, for example, GAA which is a mature peptide of alpha-glucosidase (GAA) (e.g., human GAA) comprising the amino acid sequence set forth in SEQ ID NO: 325 or a variant thereof.

Also provided are antigen-binding proteins or antibodies or antigen-binding fragments thereof comprising any of the heavy chain variable regions and/or light chain variable regions or any of the heavy chain variable region and light chain variable region combinations or any of the HCDR and LCDR combinations described above in the context of anti-hTFR:Payload scFv fusion proteins.

In an embodiment, the scFv or antigen-binding protein or antibody or antigen-binding fragment thereof comprises: (23) a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof); or (25) a HCVR comprising the HCDR1, HCDR2 and HCDR3 of a HCVR that comprises the amino acid sequence set forth in SEQ ID NO: 242 (or a variant thereof); and a LCVR comprising the LCDR1, LCDR2 and LCDR3 of a LCVR that comprises the amino acid sequence set forth in SEQ ID NO: 247 (or a variant thereof). In an embodiment, the scFv or antigen-binding protein or antibody or antigen-binding fragment thereof comprises: (23) a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof). In an embodiment, the scFv or antigen-binding protein or antibody or antigen-binding fragment thereof comprises: (25) a HCVR comprising the HCDR1, HCDR2 and HCDR3 of a HCVR that comprises the amino acid sequence set forth in SEQ ID NO: 242 (or a variant thereof); and a LCVR comprising the LCDR1, LCDR2 and LCDR3 of a LCVR that comprises the amino acid sequence set forth in SEQ ID NO: 247 (or a variant thereof). In an embodiment, the scFv or antigen-binding protein or antibody or antigen-binding fragment thereof comprises: (14) a HCVR comprising the HCDR1, HCDR2 and HCDR3 of a HCVR that comprises the amino acid sequence set forth in SEQ ID NO: 132 (or a variant thereof); and a LCVR comprising the LCDR1, LCDR2 and LCDR3 of a LCVR that comprises the amino acid sequence set forth in SEQ ID NO: 137 (or a variant thereof). In an embodiment, the scFv or antigen-binding protein or antibody or antigen-binding fragment thereof comprises: (18) a HCVR comprising the HCDR1, HCDR2 and HCDR3 of a HCVR that comprises the amino acid sequence set forth in SEQ ID NO: 172 (or a variant thereof); and a LCVR comprising the LCDR1, LCDR2 and LCDR3 of a LCVR that comprises the amino acid sequence set forth in SEQ ID NO: 177 (or a variant thereof). In an embodiment, the scFv or antigen-binding protein or antibody or antigen-binding fragment thereof comprises: (27) a HCVR comprising the HCDR1, HCDR2 and HCDR3 of a HCVR that comprises the amino acid sequence set forth in SEQ ID NO: 262 (or a variant thereof); and a LCVR comprising the LCDR1, LCDR2 and LCDR3 of a LCVR that comprises the amino acid sequence set forth in SEQ ID NO: 267 (or a variant thereof). In an embodiment, the scFv or antigen-binding protein or antibody or antigen-binding fragment thereof comprises: (28) a HCVR comprising the HCDR1, HCDR2 and HCDR3 of a HCVR that comprises the amino acid sequence set forth in SEQ ID NO: 272 (or a variant thereof); and a LCVR comprising the LCDR1, LCDR2 and LCDR3 of a LCVR that comprises the amino acid sequence set forth in SEQ ID NO: 277 (or a variant thereof).

In an embodiment, the scFv or antigen-binding protein or antibody or antigen-binding fragment thereof comprises: (w) a HCVR that comprises: an HCDR1 comprising the amino acid sequence set forth in SEQ ID NO: 223 (or a variant thereof), an HCDR2 comprising the amino acid sequence set forth in SEQ ID NO: 224 (or a variant thereof), and an HCDR3 comprising the amino acid sequence set forth in SEQ ID NO: 225 (or a variant thereof); and a LCVR that comprises: an LCDR1 comprising the amino acid sequence set forth in SEQ ID NO: 228 (or a variant thereof), an LCDR2 comprising the amino acid sequence set forth in SEQ ID NO: 229 (or a variant thereof), and an LCDR3 comprising the amino acid sequence set forth in SEQ ID NO: 230 (or a variant thereof); or (y) a HCVR that comprises: an HCDR1 comprising the amino acid sequence set forth in SEQ ID NO: 243 (or a variant thereof), an HCDR2 comprising the amino acid sequence set forth in SEQ ID NO: 244 (or a variant thereof), and an HCDR3 comprising the amino acid sequence set forth in SEQ ID NO: 245 (or a variant thereof); and a LCVR that comprises: an LCDR1 comprising the amino acid sequence set forth in SEQ ID NO: 248 (or a variant thereof), an LCDR2 comprising the amino acid sequence set forth in SEQ ID NO: 249 (or a variant thereof), and an LCDR3 comprising the amino acid sequence set forth in SEQ ID NO: 250 (or a variant thereof). In an embodiment, the scFv or antigen-binding protein or antibody or antigen-binding fragment thereof comprises: (w) a HCVR that comprises: an HCDR1 comprising the amino acid sequence set forth in SEQ ID NO: 223 (or a variant thereof), an HCDR2 comprising the amino acid sequence set forth in SEQ ID NO: 224 (or a variant thereof), and an HCDR3 comprising the amino acid sequence set forth in SEQ ID NO: 225 (or a variant thereof); and a LCVR that comprises: an LCDR1 comprising the amino acid sequence set forth in SEQ ID NO: 228 (or a variant thereof), an LCDR2 comprising the amino acid sequence set forth in SEQ ID NO: 229 (or a variant thereof), and an LCDR3 comprising the amino acid sequence set forth in SEQ ID NO: 230 (or a variant thereof). In an embodiment, the scFv or antigen-binding protein or antibody or antigen-binding fragment thereof comprises: (y) a HCVR that comprises: an HCDR1 comprising the amino acid sequence set forth in SEQ ID NO: 243 (or a variant thereof), an HCDR2 comprising the amino acid sequence set forth in SEQ ID NO: 244 (or a variant thereof), and an HCDR3 comprising the amino acid sequence set forth in SEQ ID NO: 245 (or a variant thereof); and a LCVR that comprises: an LCDR1 comprising the amino acid sequence set forth in SEQ ID NO: 248 (or a variant thereof), an LCDR2 comprising the amino acid sequence set forth in SEQ ID NO: 249 (or a variant thereof), and an LCDR3 comprising the amino acid sequence set forth in SEQ ID NO: 250 (or a variant thereof). In an embodiment, the scFv or antigen-binding protein or antibody or antigen-binding fragment thereof comprises: (n) a HCVR that comprises: an HCDR1 comprising the amino acid sequence set forth in SEQ ID NO: 133 (or a variant thereof), an HCDR2 comprising the amino acid sequence set forth in SEQ ID NO: 134 (or a variant thereof), and an HCDR3 comprising the amino acid sequence set forth in SEQ ID NO: 135 (or a variant thereof); and a LCVR that comprises: an LCDR1 comprising the amino acid sequence set forth in SEQ ID NO: 138 (or a variant thereof), an LCDR2 comprising the amino acid sequence set forth in SEQ ID NO: 139 (or a variant thereof), and an LCDR3 comprising the amino acid sequence set forth in SEQ ID NO: 140 (or a variant thereof). In an embodiment, the scFv or antigen-binding protein or antibody or antigen-binding fragment thereof comprises: (r) a HCVR that comprises: an HCDR1 comprising the amino acid sequence set forth in SEQ ID NO: 173 (or a variant thereof), an HCDR2 comprising the amino acid sequence set forth in SEQ ID NO: 174 (or a variant thereof), and an HCDR3 comprising the amino acid sequence set forth in SEQ ID NO: 175 (or a variant thereof); and a LCVR that comprises: an LCDR1 comprising the amino acid sequence set forth in SEQ ID NO: 178 (or a variant thereof), an LCDR2 comprising the amino acid sequence set forth in SEQ ID NO: 179 (or a variant thereof), and an LCDR3 comprising the amino acid sequence set forth in SEQ ID NO: 180 (or a variant thereof). In an embodiment, the scFv or antigen-binding protein or antibody or antigen-binding fragment thereof comprises: (aa) a HCVR that comprises: an HCDR1 comprising the amino acid sequence set forth in SEQ ID NO: 263 (or a variant thereof), an HCDR2 comprising the amino acid sequence set forth in SEQ ID NO: 264 (or a variant thereof), and an HCDR3 comprising the amino acid sequence set forth in SEQ ID NO: 265 (or a variant thereof); and a LCVR that comprises: an LCDR1 comprising the amino acid sequence set forth in SEQ ID NO: 268 (or a variant thereof), an LCDR2 comprising the amino acid sequence set forth in SEQ ID NO: 269 (or a variant thereof), and an LCDR3 comprising the amino acid sequence set forth in SEQ ID NO: 270 (or a variant thereof). In an embodiment, the scFv or antigen-binding protein or antibody or antigen-binding fragment thereof comprises: (ab) a HCVR that comprises: an HCDR1 comprising the amino acid sequence set forth in SEQ ID NO: 273 (or a variant thereof), an HCDR2 comprising the amino acid sequence set forth in SEQ ID NO: 274 (or a variant thereof), and an HCDR3 comprising the amino acid sequence set forth in SEQ ID NO: 275 (or a variant thereof); and a LCVR that comprises: an LCDR1 comprising the amino acid sequence set forth in SEQ ID NO: 278 (or a variant thereof), an LCDR2 comprising the amino acid sequence set forth in SEQ ID NO: 279 (or a variant thereof), and an LCDR3 comprising the amino acid sequence set forth in SEQ ID NO: 280 (or a variant thereof).

In an embodiment, the scFv or antigen-binding protein or antibody or antigen-binding fragment thereof comprises: (xxiii) a HCVR that comprises the amino acid sequence set forth in SEQ ID NO: 222 (or a variant thereof); and a LCVR that comprises the amino acid sequence set forth in SEQ ID NO: 227 (or a variant thereof); or (xxv) a HCVR that comprises the amino acid sequence set forth in SEQ ID NO: 242 (or a variant thereof); and a LCVR that comprises the amino acid sequence set forth in SEQ ID NO: 247 (or a variant thereof). In an embodiment, the scFv or antigen-binding protein or antibody or antigen-binding fragment thereof comprises: (xxiii) a HCVR that comprises the amino acid sequence set forth in SEQ ID NO: 222 (or a variant thereof); and a LCVR that comprises the amino acid sequence set forth in SEQ ID NO: 227 (or a variant thereof). In an embodiment, the scFv or antigen-binding protein or antibody or antigen-binding fragment thereof comprises: (xxv) a HCVR that comprises the amino acid sequence set forth in SEQ ID NO: 242 (or a variant thereof); and a LCVR that comprises the amino acid sequence set forth in SEQ ID NO: 247 (or a variant thereof). In an embodiment, the scFv or antigen-binding protein or antibody or antigen-binding fragment thereof comprises: (xiv) a HCVR that comprises the amino acid sequence set forth in SEQ ID NO: 132 (or a variant thereof); and a LCVR that comprises the amino acid sequence set forth in SEQ ID NO: 137 (or a variant thereof). In an embodiment, the scFv or antigen-binding protein or antibody or antigen-binding fragment thereof comprises: (xviii) a HCVR that comprises the amino acid sequence set forth in SEQ ID NO: 172 (or a variant thereof); and a LCVR that comprises the amino acid sequence set forth in SEQ ID NO: 177 (or a variant thereof). In an embodiment, the scFv or antigen-binding protein or antibody or antigen-binding fragment thereof comprises: (xxvii) a HCVR that comprises the amino acid sequence set forth in SEQ ID NO: 262 (or a variant thereof); and a LCVR that comprises the amino acid sequence set forth in SEQ ID NO: 267 (or a variant thereof). In an embodiment, the scFv or antigen-binding protein or antibody or antigen-binding fragment thereof comprises: (xxviii) a HCVR that comprises the amino acid sequence set forth in SEQ ID NO: 272 (or a variant thereof); and a LCVR that comprises the amino acid sequence set forth in SEQ ID NO: 277 (or a variant thereof).

In an embodiment, an anti-hTfR scFv provided herein, in V_{L}-(Gly₄Ser)₃ (SEQ ID NO: 538)-V_{H} format (Gly₄Ser=SEQ ID NO: 426), comprises an amino acid sequence as set forth below (optionally, an anti-hTfR scFv provided herein further includes an N-terminal LLQGSG (SEQ ID NO: 501) and/or a C-terminal HHHHHH (SEQ ID NO: 502)):

(1) 12795B
(SEQ ID NO: 427)
DIQMTQSPSSLSASVGDRVTITCRASQGIRDHFGWYQQKPGKAPKRLIYAASSLHSGVPSRESGSGSGTEFTLTISS
LQPEDFATYYCLQYDTYPLTFGGGTKVEIKGGGGSGGGGSGGGGSEVQLVESGGGLVQPGGSLRLSCATSGFTFTSY
DMKWVRQAPGLGLEWVSAISGSGGNTYYADSVKGRFTISRDNSRNTLYLQMNSLRAEDTAVYYCTRSHDFGAFDYED
YWGQGTLVTVSS
(2) 12798B (REGN17072)
(SEQ ID NO: 428)
EIVMTQSPATLSVSPGERATLSCRASQTVSSNLAWYQQKPGQAPRLLIYGSSSRATGIPARFSGSGSGTEFTLTISS
LQSEDFAVYYCQQYNNWPPYTFGQGTKLEIKGGGGSGGGGSGGGGSEVQLVESGGDLVQPGRSLRLSCAASGFTEDD
YAMHWVRQAPGKGLEWVSGISWNSATRVYADSVKGRFTISRDNAKNFLYLQMNSLRSEDTALYHCAKDMDISLGYYG
LDVWGQGTTVTVSS
(3) 12799B (REGN17073)
(SEQ ID NO: 429)
DIQMTQSPSSVSASVGDRVTITCRASQGIASWLAWYQQKPGKAPELLIYAASSLQGGVPSRESGSGSGTDFTLTISS
LQPEDFAIYYCQQANYFPWTFGQGTKVEIKGGGGSGGGGSGGGGSQITLKESGPTLVKPTQTLTLTCTFSGFSLSTS
GVGVVWIRQPPGKALEWLALIYWNDHKRYSPSLGSRLTITKDTSKNQVVLTMTNMDPVDTATYYCAHYSGSYSYYYY
GLDVWGQGTTVTVSS
(4) 12801B
(SEQ ID NO: 430)
DIQMTQSPSSLSASVGDRVTITCRASQGIRTDLGWYQQKPGKAPKRLIYAASSLQSGVPSRFSGSGSGTEFTLTISS
LRPEDFATFYCLQYNSYPLTFGGGTKVEIKGGGGSGGGGSGGGGSEVQLLESGGALVQPGGSLRLSCAASGFTFTSY
AMHWVRQAPGKGLEWVSSIRGSGGGTYSADSVKGRFTISRDNSRDTLYLQMNSVRAEDTAVYYCARSHDYGAFDFFD
YWGQGTLVTVSS
(5) 12802B
(SEQ ID NO: 431)
EIVMTQSPATLSVSPGERATLSCRASQSVSINLAWYQQKPGQAPRLLIFVASTRATGIPARFSGSGSGTEFTLTISS
LQSEDFATYYCQQYDIWPYTFGQGTKLEIKGGGGSGGGGSGGGGSQVQLVESGGGLVKPGGSLRLSCAASGFTFSDY
FMSWIRQAPGKGLEWVSYISSTGSTINYADSVKGRFTISRDNVKNSLYLQMTSLRVEDTAVYYCTRDNWNYEYWGQG
TLVTVSS
(6) 12808B
(SEQ ID NO: 432)
DIQMTQSPSSLSASVGDRVTINCRASQGIRNDLGWYQQKPGKAPKRLIYAASSLQSGVPLRFSGSGSGTEFTLTINN
LQPEDFATYYCLSHNSYPWTFGQGTKVEIKGGGGSGGGGSGGGGSQLQLQESGPGLVKPSETLSLTCTVSGESISSN
TYYWGWIRQPPGKGLEWIGSIDYSGTTNYNPSLKSRVTISVDTSRNHFSLRLRSVTAADTAVYYCAREWGNYGYYYG
MDVWGQGTTVTVSS
(7) 12812B
(SEQ ID NO: 433)
DIQMTQSPPSVSASVGDRVTITCRASQGISSWLAWYQQKPGKAPKLLIYAASSLQSGVPSRFSGSGSGTDFTLTISS
LQPEDFATYYCQQANSFPRTFGQGTKVEIKGGGGSGGGGSGGGGSQVQLVQSGAEVKKPGSSVRVSCKASRGTESSY
AISWVRQAPGQGLEWMGGIIPIFGTANYAQKFLARVTITADESTSTAYMELSSLRSEDTAVYYCAREKGWNYFDYWG
QGTLVTVSS
(8) 12816B
(SEQ ID NO: 434)
DIVMTQSPLSLPVTPGEPASISCRSSQSLLHGNGYNYLTWYLQKPGQSPQLLIYLGSNRASGVPDRESGSGSGTDET
LKISRVEAEDVGVYYCMQALQTPYTFGQGTKLEIKGGGGSGGGGSGGGGSQVQLVESGGGLVKPGGSLRLSCAASGF
TFSDYYMNWIRQAPGKGLEWVSYISSSGTTIYYADSVKGRFTISRDNAKKSLYLEMNSLRAEDTAVYYCAREGYGND
YYYYGIDVWGQGTTVTVSS
(9) 12833B
(SEQ ID NO: 435)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKGGGGSGGGGSGGGGSQVQLVESGGGVVQPGRSLRLSCAASGFTESS
FGMHWVRQAPGKGLEWVIFISYDGSDKYYADSVKGRFAISRDSSKNTLYLQMNSLRAEDTAVYYCAKENGILTDSYG
MDVWGQGTTVTVSS
(10) 12834B
(SEQ ID NO: 436)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKGGGGSGGGGSGGGGSQVQLVQSGAEVKKPGASVKVSCKASGYTFTS
YGISWVRQAPGQGLEWMGWISVYHGNTNYAQKFQGRVTMTTDTSTSTAYMELRSLRSDDTAVYYCAREGYYDFWSGY
YPFDYWGQGTLVTVSS
(11) 12835B
(SEQ ID NO: 437)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKGGGGSGGGGSGGGGSEVQLVESGGGLIQPGGSLRLSCEASGFTERN
YEMNWVRQAPGKGLEWVSYISSSGNMKDYAESVKGRFTISRDNVKNSLQLQMNSLRVEDTAVYYCARDEFPYGMDVW
GQGTTVTVSS
(12) 12839B (REGN17074)
(SEQ ID NO: 438)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKGGGGSGGGGSGGGGSQVQLVESGGGVVQPGRSLRLSCAASGFPFSN
YVMYWVRQAPGKGLEWVALIFFDGKKNYHADSVKGRFTITRDNSKNMLYLQMNSLRPEDAAVYYCAKIHCPNGVCYK
GYYGMDVWGQGTTVTVSS
(13) 12841B
(SEQ ID NO: 439)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKGGGGSGGGGSGGGGSEVQLVESGGGLVQPGGSLRLSCAASGFTFSN
YWMNWVRQAPGKGLEWVANIKEDGGKKLYVDSVKGRFTISRDNAKNSLFLQMNSLRAEDTAVYYCAREDTTLVVDYY
YYGMDVWGQGTTVTVSS
(14) 12843B (REGN17075)
(SEQ ID NO: 440)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKGGGGSGGGGSGGGGSEVQLVESGGGLVQPGGSLRLSCAASGFTENI
FEMNWVRQAPGKGLEWISYISSRGTTTYYADSVRGRFTISRDNAKNSLYLQMNSLRAEDTAVYYCARDYEATIPFDF
WGQGTLVTVSS
(15) 12844B
(SEQ ID NO: 441)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKGGGGSGGGGSGGGGSEVQLVESGGSVVRPGGSLRLSCEASGFTEDD
YGMSWVRQDPGKGLEWVSGINWNGDRTNYADSVKGRFIISRDNAKNSVYLQMNSLRAEDSALYHCARDQGLGVAATL
DYWGQGTLVTVSS
(16) 12845B (REGN17076)
(SEQ ID NO: 442)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKGGGGSGGGGSGGGGSEVQLVESGGGLVQPGGSLRLSCAASGFTVSN
YEMNWVRQAPGKGLEWVSYISSSTSNIYYADSVKGRFTISRDNAENSLYLQMNSLRVEDTAVYYCVRDGIVVVPVGR
GYYYYGLDVWGQGTTVTVSS
(17) 12847B (REGN17077)
(SEQ ID NO: 443)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKGGGGSGGGGSGGGGSEVQLVESGGGLVQPGRSLRLSCAASGFTFDD
YAMNWVRQAPGKGLEWVSGISWSSGSMDYADSVKGRFTISRDNAKNSLYLQMNSLRTEDTALYYCAKAREVGDYYGM
DVWGQGTTVTVSS
(18) 12848B
(SEQ ID NO: 444)
EIVLTQSPGTLSLSPGERATLSCRASQSVSSSYLAWYQQKPGQAPRLLIYGASSRATGIPDRFSGSGSGTDFTLTIS
RLEPEDFAVYYCQQYGSSPWTFGQGTKVEIKGGGGSGGGGSGGGGSEVQLVESGGGLVQPGRSLTLSCAASGFTFDN
FGMHWVRQGPGKGLEWVSGLTWNSGVIGYADSVKGRFTISRDNAKNSLYLQMNSLRPEDTALYYCAKDIRNYGPFDY
WGQGTLVTVSS
(19) 12850B
(SEQ ID NO: 445)
EIVLTQSPGTLSLSPGERATLSCRASQSVSSSYLAWYQQKPGQAPRLLIYGASSRATGIPDRFSGSGSGTDFTLTIS
RLEPEDFAVYYCQQYGSSPWTFGQGTKVEIKGGGGSGGGGSGGGGSQVQLVQSGAEVKKPGSSVKVSCKASGGTENT
YAITWVRQAPGOGLEWMGGIIPISGIAEYAQKFQGRVTITTDDSSTTAYMELNSLRSEDTAVYYCASWNYALYYFYG
MDVWGRGTTVTVSS
(20) 31863B
(SEQ ID NO: 446)
DIQMTQSPSSLSASIGDRVTITCRASQGISNYLAWYQQKPGKVPKLLIYAASTLQSGVPSRFSGSGSGTDFTLTISS
LQPEDVATYYCONHNSVPLTFGGGTKVEIKGGGGSGGGGSGGGGSEVQLVESGGGLVQPGGSLRLSCAASGFTENSY
AMTWVRQAPGKGLEWVSFIGGSTGNTYYAGSVKGRFTISSDNSKKTLYLQMNSLRAEDTAVYYCAKGGAARRMEYFQ
HWGQGTLVTVSS
(21) 31874B
(SEQ ID NO: 447)
DIQMTQSPSSLSASVGDRVTITCRASQGISNYLAWYQQKPGKVPNLLIYAASTLQSGVPSRFSGSGSGTDFTLTISS
LQPEDVATYYCQKYNSAPLTFGGGTKVEIKGGGGSGGGGSGGGGSEVQLVESGGGLVQPGGSLRLSCAASGFAFSSY
AMTWVRQAPGKGLEWVSVISGTGGSTYYADSVKGRFTISRDNSKNTLYLQMNSLRAEDTAVYYCAKGGAARRMEYFQ
YWGQGTLVTVSS
(22) 69261
(SEQ ID NO: 448)
DIVMTQSPLSLPVTPGEPASISCRSSQSLLHSNGYNYLDWYLQKPGQSPQFLIYLGSNRASGVPDRFSGSGSGTDFT
LKINRVEAEDVGVYYCMQALQTPYTFGQGTKLEIKGGGGSGGGGSGGGGSQVQLVESGGGLVKPGGSLRLSCAASGF
TFSVYYMNWIRQAPGKGLEWVSYISSSGSTIYYADSVKGRFTISRDNAKNSLYLQMNSLRAEDTAVYYCGREGYSGT
YSYYGMDVWGQGTTVTVSS
(23) 69263
(SEQ ID NO: 449)
DIQMTQSPSSLSASVGDRVTITCRASQDISHYSAWYQQKPGKLPNLLIYAASTLQSGVPSRESGSGSGTDESLTTSS
LQPEDVATYYCQKYNSVPLTFGGGTKVEIKGGGGSGGGGSGGGGSEVQLVESGGGLVQPGRSLRLSCAVSGFTEDDY
AMHWVRQAPGKGLEWVSGISWNSGTRGYADSVKGRFTISRDNAKNSLYLQMNSLRGEDTALYYCVKDITISPNYYGM
DVWGQGTTVTVSS
(24) 69305
(SEQ ID NO: 450)
DIQMTQSPSSLSASVGDRVTITCRASQSIDRYLNWYRQKPGKAPKLLIYTTSSLQSGVPSRFSGSGSGTDFTLTLSS
LQPEDFATYYCQQSYSPPLTFGGGTKVEIKGGGGSGGGGSGGGGSQVQLVESGGGVVQPGRSLRLSCAASGFTFSSY
GMHWVRQAPGKGLEWVAVIWYDGSNKYYADSVKGRFTISRDISKNTLYLQMNSLRAEDTAVYYCAGQLDLFFDYWGQ
GTLVTVSS
(25) 69307
(SEQ ID NO: 451)
DIQMTQSPSSVSASVGDRVTITCRASQGISSWLAWYQQKPGKAPKLLIYAASSLQSGVPSRFSGSGSGTDFTLTISS
LQPEDFATYYCQKADSLPYAFGQGTKLEIKGGGGSGGGGSGGGGSEVQLVESGGGLVQPGGSLRLSCTASGFTFSNY
WMTWVRQAPGKGLEWVANIKEDGSEKEYVDSVKGRFTISRDNAKNSLYLQMNSLRGEDTAVYYCARDGEQLVDYYYY
YVMDVWGQGTTVTVSS
(26) 69323
(SEQ ID NO: 452)
DIQMTQSPSSLSASVGDRVTITCRASQSISSYLNWYQQKPGKAPKVLIYAASSLQSGVPSRFSGSGSGTDFTLTISS
LQPEDFATYYCQQSYSIPLTFGGGTKVEIKGGGGSGGGGSGGGGSEVQLVESGGGLVQPGRSLRLSCAASGFTEDDY
AMHWVRQAPGKGLEWVSGISWNSGYIGYADSVKGRFTISRDNAENSLHLQMNSLRAEDTALYYCARGGSTLVRGVKG
GYYGMDVWGQGTTVTVSS
(27) 69326
(SEQ ID NO: 453)
EIVMTQSPATLSVSPGERATLSCRASQSVSSNFAWYQQKPGQAPRLLIYSASSRATGIPVRFSGSGSGTEFTLTISS
LQSEDFAVYYCQQYNIWPRTFGQGTKVEIKGGGGSGGGGSGGGGSEVQLVESGGGLVQPGGSLRLSCAVSGFIFSSY
EMNWVRQAPGKGLEWVSYISSSGSTIFYADSVKGRFTISRDNAKNSLYLQMNSLRAEDTAVYYCVSGVVLFDVWGQG
TMVTVSS
(28) 69329
(SEQ ID NO: 454)
DIQMTQSPSSVSASVGDRVTITCRASQGISSWLAWYQQKPGKAPKLLIYAASSLQSGVPSRFSGSGSGTDFTLTISS
LQPEDFATYYCQKANSFPYTFGQGTKLEIKGGGGSGGGGSGGGGSEVQLVESGGGLVQPGGSLRLSCAASGFTFSNY
WMTWVRQAPGKGLEWVANIKEDGSEKDYVDSVKGRFTISRDNAKNSLYLQMNSLRGEDTAVYYCARDGEQLVDYYYY
YVMDVWGQGTTVTVSS
(29) 69331
(SEQ ID NO: 455)
DIQLTQSPSSLSASVGDRVTITCWASQGISSYLAWYQQKPGKAPKLLIYAASTLQSGVPSRFSGSGSGTEFTLTISS
LQPEDFATYYCQQLNSYPLTFGGGTKVEIKGGGGSGGGGSGGGGSQVQLVESGGGVVQPGRSLRLSCIASGFTFSVY
GIHWVRQAPGKGLEWMAVISHDGNIKHYADSVKGRFTISRDNSKNTLYLQINSLRTEDTAVYYCAKDTWNSLDTEDI
WGQGTMVTVSS
(30) 69332
(SEQ ID NO: 456)
AIQMTQSPSSLSASVGDRVTITCRASQGIRNDLGWYQQKPGKAPKLLIYAASTLQSGVPSRFSGSGSGTDFTLTISS
LQPEDFATYYCLQDYNYPFTFGPGTKVDIKGGGGSGGGGSGGGGSQVTLRESGPALVKPSQTLTLTCTFSGFSLNTY
GMFVSWIRQPPGKALEWLAHIHWDDDKYYSTSLKTRLTISKDTSKNQVVLTMTNMDPVDTATYYCARGHNNLNYIIH
WGQGTLVTVSS
(31) 69340
(SEQ ID NO: 457)
EIVLTQSPATLSLSPGERATLSCRASQSVSSYLAWYQQKPGQAPRLLIHDVSNRATGIPARFSGSGSGTDFTLTISS
LEPEDFVVYYCQQRSDWPITFGQGTRLEIKGGGGSGGGGSGGGGSEVQLVESGGGLVQPGRSLRLSCAASGFTFDDK
AMHWVRQVPGKGLEWISGISWNSGTIGYADSVKGRFIISRDNAKNSLYLQMNSLRAEDTALYYCAKDGDTSGWYWYG
LDVWGQGTTVTVSS
(32) 69348
(SEQ ID NO: 458)
DIQMTQSPSSLSASVGDRVTITCRASQSIRNVLGWFQQKPGKAPQRLIYAASSLQSGVPSRFSGSGSGTEFTLTISS
LQPEDFATYYCLQHNFYPLTFGGGTKVEIKGGGGSGGGGSGGGGSQVQLVESGGGVVQPGRSLRLSCAASGFTFTTY
GMHWVRQAPGKGLEWVAVIWYDGSNKYYGDSVKGRFTISRDNSKNTLYLQMNSLRVDDTAVYYCTRTHGYTRSSDGF
DYWGQGTLVTVSS; however, provided herein are such fusions that are in the
format V_{H}-(Gly₄Ser)₃ (SEQ ID NO: 538)-V_{L}(Gly₄Ser = SEQ ID NO: 426).

In an embodiment, an anti-hTfR scFv comprises an amino acid sequence as set forth in SEQ ID NO: 443 or SEQ ID NO: 440. In an embodiment, an anti-hTfR scFv comprises an amino acid sequence as set forth in SEQ ID NO: 443. In an embodiment, an anti-hTfR scFv comprises an amino acid sequence as set forth in SEQ ID NO: 440. In an embodiment, an anti-hTfR scFv comprises an amino acid sequence as set forth in SEQ ID NO: 429. In an embodiment, an anti-hTfR scFv comprises an amino acid sequence as set forth in SEQ ID NO: 433. In an embodiment, an anti-hTfR scFv comprises an amino acid sequence as set forth in SEQ ID NO: 442. In an embodiment, an anti-hTfR scFv comprises an amino acid sequence as set forth in SEQ ID NO: 438.

In an embodiment, an anti-hTfR scFv:GAA fusion provided herein comprises the amino acid sequence:

(SEQ ID NO: 392)
(1) 12795B
DIQMTQSPSSLSASVGDRVTITCRASQGIRDHFGWYQQKPGKAPKRLIYAASSLHSGVPSRFSGSGSGTEFTLTISS
LQPEDFATYYCLQYDTYPLTFGGGTKVEIKGGGGSGGGGSGGGGSEVQLVESGGGLVQPGGSLRLSCATSGFTFTSY
DMKWVRQAPGLGLEWVSAISGSGGNTYYADSVKGRFTISRDNSRNTLYLQMNSLRAEDTAVYYCTRSHDFGAFDYED
YWGQGTLVTVSSGGGGSGGGGSAHPGRPRAVPTQCDVPPNSREDCAPDKAITQEQCEARGCCYIPAKQGLQGAQMGQ
PWCFFPPSYPSYKLENLSSSEMGYTATLTRTTPTFFPKDILTLRLDVMMETENRLHFTIKDPANRRYEVPLETPHVH
SRAPSPLYSVEFSEEPFGVIVRRQLDGRVLLNTTVAPLFFADQFLQLSTSLPSQYITGLAEHLSPLMLSTSWTRITL
WNRDLAPTPGANLYGSHPFYLALEDGGSAHGVFLLNSNAMDVVLQPSPALSWRSTGGILDVYIFLGPEPKSVVQQYL
DVVGYPFMPPYWGLGFHLCRWGYSSTAITRQVVENMTRAHFPLDVQWNDLDYMDSRRDFTENKDGFRDFPAMVQELH
QGGRRYMMIVDPAISSSGPAGSYRPYDEGLRRGVFITNETGQPLIGKVWPGSTAFPDFTNPTALAWWEDMVAEFHDQ
VPFDGMWIDMNEPSNFIRGSEDGCPNNELENPPYVPGVVGGTLQAATICASSHQFLSTHYNLHNLYGLTEAIASHRA
LVKARGTRPFVISRSTFAGHGRYAGHWTGDVWSSWEQLASSVPEILQFNLLGVPLVGADVCGFLGNTSEELCVRWTQ
LGAFYPFMRNHNSLLSLPQEPYSFSEPAQQAMRKALTLRYALLPHLYTLFHQAHVAGETVARPLFLEFPKDSSTWTV
DHQLLWGEALLITPVLQAGKAEVTGYFPLGTWYDLQTVPVEALGSLPPPPAAPREPAIHSEGQWVTLPAPLDTINVH
LRAGYIIPLQGPGLTTTESRQQPMALAVALTKGGEARGELFWDDGESLEVLERGAYTQVIFLARNNTIVNELVRVTS
EGAGLQLQKVTVLGVATAPQQVLSNGVPVSNFTYSPDTKVLDICVSLLMGEQFLVSWC
(2) 12798B (REGN16818)
(SEQ ID NO: 393)
EIVMTQSPATLSVSPGERATLSCRASQTVSSNLAWYQQKPGQAPRLLIYGSSSRATGIPARFSGSGSGTEFTLTISS
LQSEDFAVYYCQQYNNWPPYTFGQGTKLEIKGGGGSGGGGSGGGGSEVQLVESGGDLVQPGRSLRLSCAASGFTFDD
YAMHWVRQAPGKGLEWVSGISWNSATRVYADSVKGRFTISRDNAKNFLYLQMNSLRSEDTALYHCAKDMDISLGYYG
LDVWGQGTTVTVSSGGGGSGGGGSAHPGRPRAVPTQCDVPPNSREDCAPDKAITQEQCEARGCCYIPAKQGLQGAQM
GQPWCFFPPSYPSYKLENLSSSEMGYTATLTRTTPTFFPKDILTLRLDVMMETENRLHFTIKDPANRRYEVPLETPH
VHSRAPSPLYSVEFSEEPFGVIVRRQLDGRVLLNTTVAPLFFADQFLQLSTSLPSQYITGLAEHLSPLMLSTSWTRI
TLWNRDLAPTPGANLYGSHPFYLALEDGGSAHGVFLLNSNAMDVVLQPSPALSWRSTGGILDVYIFLGPEPKSVVQQ
YLDVVGYPFMPPYWGLGFHLCRWGYSSTAITRQVVENMTRAHFPLDVQWNDLDYMDSRRDFTFNKDGFRDFPAMVQE
LHQGGRRYMMIVDPAISSSGPAGSYRPYDEGLRRGVFITNETGQPLIGKVWPGSTAFPDFTNPTALAWWEDMVAEFH
DQVPFDGMWIDMNEPSNFIRGSEDGCPNNELENPPYVPGVVGGTLQAATICASSHQFLSTHYNLHNLYGLTEAIASH
RALVKARGTRPFVISRSTFAGHGRYAGHWTGDVWSSWEQLASSVPEILQFNLLGVPLVGADVCGELGNTSEELCVRW
TQLGAFYPFMRNHNSLLSLPQEPYSFSEPAQQAMRKALTLRYALLPHLYTLFHQAHVAGETVARPLFLEFPKDSSTW
TVDHQLLWGEALLITPVLQAGKAEVTGYFPLGTWYDLQTVPVEALGSLPPPPAAPREPAIHSEGQWVTLPAPLDTIN
VHLRAGYIIPLQGPGLTTTESRQQPMALAVALTKGGEARGELFWDDGESLEVLERGAYTQVIFLARNNTIVNELVRV
TSEGAGLQLQKVTVLGVATAPQQVLSNGVPVSNFTYSPDTKVLDICVSLLMGEQFLVSWC
(3) 12799B (REGN16819)
(SEQ ID NO: 394)
DIQMTQSPSSVSASVGDRVTITCRASQGIASWLAWYQQKPGKAPELLIYAASSLQGGVPSRFSGSGSGTDFTLTISS
LQPEDFAIYYCQQANYFPWTFGQGTKVEIKGGGGSGGGGSGGGGSQITLKESGPTLVKPTQTLTLTCTFSGFSLSTS
GVGVVWIRQPPGKALEWLALIYWNDHKRYSPSLGSRLTITKDTSKNQVVLTMTNMDPVDTATYYCAHYSGSYSYYYY
GLDVWGQGTTVTVSSGGGGSGGGGSAHPGRPRAVPTQCDVPPNSREDCAPDKAITQEQCEARGCCYIPAKQGLQGAQ
MGQPWCFFPPSYPSYKLENLSSSEMGYTATLTRTTPTFFPKDILTLRLDVMMETENRLHFTIKDPANRRYEVPLETP
HVHSRAPSPLYSVEFSEEPFGVIVRRQLDGRVLLNTTVAPLFFADQFLQLSTSLPSQYITGLAEHLSPLMLSTSWTR
ITLWNRDLAPTPGANLYGSHPFYLALEDGGSAHGVFLLNSNAMDVVLQPSPALSWRSTGGILDVYIFLGPEPKSVVQ
QYLDVVGYPFMPPYWGLGFHLCRWGYSSTAITRQVVENMTRAHFPLDVQWNDLDYMDSRRDFTENKDGFRDFPAMVQ
ELHQGGRRYMMIVDPAISSSGPAGSYRPYDEGLRRGVFITNETGQPLIGKVWPGSTAFPDFTNPTALAWWEDMVAEF
HDQVPFDGMWIDMNEPSNFIRGSEDGCPNNELENPPYVPGVVGGTLQAATICASSHQFLSTHYNLHNLYGLTEAIAS
HRALVKARGTRPFVISRSTFAGHGRYAGHWTGDVWSSWEQLASSVPEILQFNLLGVPLVGADVCGFLGNTSEELCVR
WTQLGAFYPFMRNHNSLLSLPQEPYSFSEPAQQAMRKALTLRYALLPHLYTLFHQAHVAGETVARPLFLEFPKDSST
WTVDHQLLWGEALLITPVLQAGKAEVTGYFPLGTWYDLQTVPVEALGSLPPPPAAPREPAIHSEGQWVTLPAPLDTI
NVHLRAGYIIPLQGPGLTTTESRQQPMALAVALTKGGEARGELFWDDGESLEVLERGAYTQVIFLARNNTIVNELVR
VTSEGAGLQLQKVTVLGVATAPQQVLSNGVPVSNFTYSPDTKVLDICVSLLMGEQFLVSWC
(4) 12801B
(SEQ ID NO: 395)
DIQMTQSPSSLSASVGDRVTITCRASQGIRTDLGWYQQKPGKAPKRLIYAASSLQSGVPSRFSGSGSGTEFTLTISS
LRPEDFATFYCLQYNSYPLTFGGGTKVEIKGGGGSGGGGSGGGGSEVQLLESGGALVQPGGSLRLSCAASGFTFTSY
AMHWVRQAPGKGLEWVSSIRGSGGGTYSADSVKGRFTISRDNSRDTLYLQMNSVRAEDTAVYYCARSHDYGAFDFFD
YWGQGTLVTVSSGGGGSGGGGSAHPGRPRAVPTQCDVPPNSREDCAPDKAITQEQCEARGCCYIPAKQGLQGAQMGQ
PWCFFPPSYPSYKLENLSSSEMGYTATLTRTTPTFFPKDILTLRLDVMMETENRLHFTIKDPANRRYEVPLETPHVH
SRAPSPLYSVEFSEEPFGVIVRRQLDGRVLLNTTVAPLFFADQFLQLSTSLPSQYITGLAEHLSPLMLSTSWTRITL
WNRDLAPTPGANLYGSHPFYLALEDGGSAHGVFLLNSNAMDVVLQPSPALSWRSTGGILDVYIFLGPEPKSVVQQYL
DVVGYPFMPPYWGLGFHLCRWGYSSTAITRQVVENMTRAHFPLDVQWNDLDYMDSRRDFTENKDGFRDFPAMVQELH
QGGRRYMMIVDPAISSSGPAGSYRPYDEGLRRGVFITNETGQPLIGKVWPGSTAFPDFTNPTALAWWEDMVAEFHDQ
VPFDGMWIDMNEPSNFIRGSEDGCPNNELENPPYVPGVVGGTLQAATICASSHQFLSTHYNLHNLYGLTEAIASHRA
LVKARGTRPFVISRSTFAGHGRYAGHWTGDVWSSWEQLASSVPEILQFNLLGVPLVGADVCGFLGNTSEELCVRWTQ
LGAFYPFMRNHNSLLSLPQEPYSFSEPAQQAMRKALTLRYALLPHLYTLFHQAHVAGETVARPLFLEFPKDSSTWTV
DHQLLWGEALLITPVLQAGKAEVTGYFPLGTWYDLQTVPVEALGSLPPPPAAPREPAIHSEGQWVTLPAPLDTINVH
LRAGYIIPLQGPGLTTTESRQQPMALAVALTKGGEARGELFWDDGESLEVLERGAYTQVIFLARNNTIVNELVRVTS
EGAGLQLQKVTVLGVATAPQQVLSNGVPVSNFTYSPDTKVLDICVSLLMGEQFLVSWC
(5) 12802B (REGN16820)
(SEQ ID NO: 396)
EIVMTQSPATLSVSPGERATLSCRASQSVSINLAWYQQKPGQAPRLLIFVASTRATGIPARFSGSGSGTEFTLTISS
LQSEDFATYYCQQYDIWPYTFGQGTKLEIKGGGGSGGGGSGGGGSQVQLVESGGGLVKPGGSLRLSCAASGFTFSDY
FMSWIRQAPGKGLEWVSYISSTGSTINYADSVKGRFTISRDNVKNSLYLQMTSLRVEDTAVYYCTRDNWNYEYWGQG
TLVTVSSGGGGSGGGGSAHPGRPRAVPTQCDVPPNSRFDCAPDKAITQEQCEARGCCYIPAKQGLQGAQMGQPWCFF
PPSYPSYKLENLSSSEMGYTATLTRTTPTFFPKDILTLRLDVMMETENRLHFTIKDPANRRYEVPLETPHVHSRAPS
PLYSVEFSEEPFGVIVRRQLDGRVLLNTTVAPLFFADQFLQLSTSLPSQYITGLAEHLSPLMLSTSWTRITLWNRDL
APTPGANLYGSHPFYLALEDGGSAHGVFLLNSNAMDVVLQPSPALSWRSTGGILDVYIFLGPEPKSVVQQYLDVVGY
PFMPPYWGLGFHLCRWGYSSTAITRQVVENMTRAHFPLDVQWNDLDYMDSRRDFTFNKDGFRDFPAMVQELHQGGRR
YMMIVDPAISSSGPAGSYRPYDEGLRRGVFITNETGQPLIGKVWPGSTAFPDFTNPTALAWWEDMVAEFHDQVPFDG
MWIDMNEPSNFIRGSEDGCPNNELENPPYVPGVVGGTLQAATICASSHQFLSTHYNLHNLYGLTEAIASHRALVKAR
GTRPFVISRSTFAGHGRYAGHWTGDVWSSWEQLASSVPEILQFNLLGVPLVGADVCGFLGNTSEELCVRWTQLGAFY
PFMRNHNSLLSLPQEPYSFSEPAQQAMRKALTLRYALLPHLYTLFHQAHVAGETVARPLFLEFPKDSSTWTVDHQLL
WGEALLITPVLQAGKAEVTGYFPLGTWYDLQTVPVEALGSLPPPPAAPREPAIHSEGQWVTLPAPLDTINVHLRAGY
IIPLQGPGLTTTESRQQPMALAVALTKGGEARGELFWDDGESLEVLERGAYTQVIFLARNNTIVNELVRVTSEGAGL
QLQKVTVLGVATAPQQVLSNGVPVSNFTYSPDTKVLDICVSLLMGEQFLVSWC
(6) 12808B
(SEQ ID NO: 397)
DIQMTQSPSSLSASVGDRVTINCRASQGIRNDLGWYQQKPGKAPKRLIYAASSLQSGVPLRFSGSGSGTEFTLTINN
LQPEDFATYYCLSHNSYPWTFGQGTKVEIKGGGGSGGGGSGGGGSQLQLQESGPGLVKPSETLSLTCTVSGESISSN
TYYWGWIRQPPGKGLEWIGSIDYSGTTNYNPSLKSRVTISVDTSRNHFSLRLRSVTAADTAVYYCAREWGNYGYYYG
MDVWGQGTTVTVSSGGGGSGGGGSAHPGRPRAVPTQCDVPPNSREDCAPDKAITQEQCEARGCCYIPAKQGLQGAQM
GQPWCFFPPSYPSYKLENLSSSEMGYTATLTRTTPTFFPKDILTLRLDVMMETENRLHFTIKDPANRRYEVPLETPH
VHSRAPSPLYSVEFSEEPFGVIVRRQLDGRVLLNTTVAPLFFADQFLQLSTSLPSQYITGLAEHLSPLMLSTSWTRI
TLWNRDLAPTPGANLYGSHPFYLALEDGGSAHGVFLLNSNAMDVVLQPSPALSWRSTGGILDVYIFLGPEPKSVVQQ
YLDVVGYPFMPPYWGLGFHLCRWGYSSTAITRQVVENMTRAHFPLDVQWNDLDYMDSRRDFTENKDGERDFPAMVQE
LHQGGRRYMMIVDPAISSSGPAGSYRPYDEGLRRGVFITNETGQPLIGKVWPGSTAFPDFTNPTALAWWEDMVAEFH
DQVPFDGMWIDMNEPSNFIRGSEDGCPNNELENPPYVPGVVGGTLQAATICASSHQFLSTHYNLHNLYGLTEAIASH
RALVKARGTRPFVISRSTFAGHGRYAGHWTGDVWSSWEQLASSVPEILQFNLLGVPLVGADVCGFLGNTSEELCVRW
TQLGAFYPFMRNHNSLLSLPQEPYSFSEPAQQAMRKALTLRYALLPHLYTLFHQAHVAGETVARPLFLEFPKDSSTW
TVDHQLLWGEALLITPVLQAGKAEVTGYFPLGTWYDLQTVPVEALGSLPPPPAAPREPAIHSEGQWVTLPAPLDTIN
VHLRAGYIIPLQGPGLTTTESRQQPMALAVALTKGGEARGELFWDDGESLEVLERGAYTQVIFLARNNTIVNELVRV
TSEGAGLQLQKVTVLGVATAPQQVLSNGVPVSNFTYSPDTKVLDICVSLLMGEQFLVSWC
(7) 12812B (REGN16821)
(SEQ ID NO: 398)
DIQMTQSPPSVSASVGDRVTITCRASQGISSWLAWYQQKPGKAPKLLIYAASSLQSGVPSRFSGSGSGTDFTLTISS
LQPEDFATYYCQQANSFPRTFGQGTKVEIKGGGGSGGGGSGGGGSQVQLVQSGAEVKKPGSSVRVSCKASRGTESSY
AISWVRQAPGQGLEWMGGIIPIFGTANYAQKFLARVTITADESTSTAYMELSSLRSEDTAVYYCAREKGWNYFDYWG
QGTLVTVSSGGGGSGGGGSAHPGRPRAVPTQCDVPPNSREDCAPDKAITQEQCEARGCCYIPAKQGLQGAQMGQPWC
FFPPSYPSYKLENLSSSEMGYTATLTRTTPTFFPKDILTLRLDVMMETENRLHFTIKDPANRRYEVPLETPHVHSRA
PSPLYSVEFSEEPFGVIVRRQLDGRVLLNTTVAPLFFADQFLQLSTSLPSQYITGLAEHLSPLMLSTSWTRITLWNR
DLAPTPGANLYGSHPFYLALEDGGSAHGVFLLNSNAMDVVLQPSPALSWRSTGGILDVYIFLGPEPKSVVQQYLDVV
GYPFMPPYWGLGFHLCRWGYSSTAITRQVVENMTRAHFPLDVQWNDLDYMDSRRDFTENKDGFRDFPAMVQELHOGG
RRYMMIVDPAISSSGPAGSYRPYDEGLRRGVFITNETGQPLIGKVWPGSTAFPDFTNPTALAWWEDMVAEFHDQVPF
DGMWIDMNEPSNFIRGSEDGCPNNELENPPYVPGVVGGTLQAATICASSHQFLSTHYNLHNLYGLTEAIASHRALVK
ARGTRPFVISRSTFAGHGRYAGHWTGDVWSSWEQLASSVPEILQFNLLGVPLVGADVCGFLGNTSEELCVRWTQLGA
FYPFMRNHNSLLSLPQEPYSFSEPAQQAMRKALTLRYALLPHLYTLFHQAHVAGETVARPLFLEFPKDSSTWTVDHQ
LLWGEALLITPVLQAGKAEVTGYFPLGTWYDLQTVPVEALGSLPPPPAAPREPAIHSEGQWVTLPAPLDTINVHLRA
GYIIPLQGPGLTTTESRQQPMALAVALTKGGEARGELFWDDGESLEVLERGAYTQVIFLARNNTIVNELVRVTSEGA
GLQLQKVTVLGVATAPQQVLSNGVPVSNFTYSPDTKVLDICVSLLMGEQFLVSWC
(8) 12816B
(SEQ ID NO: 399)
DIVMTQSPLSLPVTPGEPASISCRSSQSLLHGNGYNYLTWYLQKPGQSPQLLIYLGSNRASGVPDRESGSGSGTDET
LKISRVEAEDVGVYYCMQALQTPYTFGQGTKLEIKGGGGSGGGGSGGGGSQVQLVESGGGLVKPGGSLRLSCAASGF
TFSDYYMNWIRQAPGKGLEWVSYISSSGTTIYYADSVKGRFTISRDNAKKSLYLEMNSLRAEDTAVYYCAREGYGND
YYYYGIDVWGQGTTVTVSSGGGGSGGGGSAHPGRPRAVPTQCDVPPNSREDCAPDKAITQEQCEARGCCYIPAKQGL
QGAQMGQPWCFFPPSYPSYKLENLSSSEMGYTATLTRTTPTFFPKDILTLRLDVMMETENRLHFTIKDPANRRYEVP
LETPHVHSRAPSPLYSVEFSEEPFGVIVRRQLDGRVLLNTTVAPLFFADQFLQLSTSLPSQYITGLAEHLSPLMLST
SWTRITLWNRDLAPTPGANLYGSHPFYLALEDGGSAHGVFLLNSNAMDVVLQPSPALSWRSTGGILDVYIFLGPEPK
SVVQQYLDVVGYPFMPPYWGLGFHLCRWGYSSTAITRQVVENMTRAHFPLDVQWNDLDYMDSRRDFTENKDGERDFP
AMVQELHQGGRRYMMIVDPAISSSGPAGSYRPYDEGLRRGVFITNETGQPLIGKVWPGSTAFPDFTNPTALAWWEDM
VAEFHDQVPFDGMWIDMNEPSNFIRGSEDGCPNNELENPPYVPGVVGGTLQAATICASSHQFLSTHYNLHNLYGLTE
AIASHRALVKARGTRPFVISRSTFAGHGRYAGHWTGDVWSSWEQLASSVPEILQFNLLGVPLVGADVCGELGNTSEE
LCVRWTQLGAFYPFMRNHNSLLSLPQEPYSFSEPAQQAMRKALTLRYALLPHLYTLFHQAHVAGETVARPLFLEFPK
DSSTWTVDHQLLWGEALLITPVLQAGKAEVTGYFPLGTWYDLQTVPVEALGSLPPPPAAPREPAIHSEGQWVTLPAP
LDTINVHLRAGYIIPLQGPGLTTTESRQQPMALAVALTKGGEARGELFWDDGESLEVLERGAYTQVIFLARNNTIVN
ELVRVTSEGAGLQLQKVTVLGVATAPQQVLSNGVPVSNFTYSPDTKVLDICVSLLMGEQFLVSWC
(9) 12833B
(SEQ ID NO: 400)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKGGGGSGGGGSGGGGSQVQLVESGGGVVQPGRSLRLSCAASGFTFSS
FGMHWVRQAPGKGLEWVIFISYDGSDKYYADSVKGRFAISRDSSKNTLYLQMNSLRAEDTAVYYCAKENGILTDSYG
MDVWGQGTTVTVSSGGGGSGGGGSAHPGRPRAVPTQCDVPPNSREDCAPDKAITQEQCEARGCCYIPAKQGLQGAQM
GQPWCFFPPSYPSYKLENLSSSEMGYTATLTRTTPTFFPKDILTLRLDVMMETENRLHFTIKDPANRRYEVPLETPH
VHSRAPSPLYSVEFSEEPFGVIVRRQLDGRVLLNTTVAPLFFADQFLQLSTSLPSQYITGLAEHLSPLMLSTSWTRI
TLWNRDLAPTPGANLYGSHPFYLALEDGGSAHGVFLLNSNAMDVVLQPSPALSWRSTGGILDVYIFLGPEPKSVVQQ
YLDVVGYPFMPPYWGLGFHLCRWGYSSTAITRQVVENMTRAHFPLDVQWNDLDYMDSRRDFTENKDGFRDFPAMVQE
LHQGGRRYMMIVDPAISSSGPAGSYRPYDEGLRRGVFITNETGQPLIGKVWPGSTAFPDFTNPTALAWWEDMVAEFH
DQVPFDGMWIDMNEPSNFIRGSEDGCPNNELENPPYVPGVVGGTLQAATICASSHQFLSTHYNLHNLYGLTEAIASH
RALVKARGTRPFVISRSTFAGHGRYAGHWTGDVWSSWEQLASSVPEILQFNLLGVPLVGADVCGELGNTSEELCVRW
TQLGAFYPFMRNHNSLLSLPQEPYSFSEPAQQAMRKALTLRYALLPHLYTLFHQAHVAGETVARPLFLEFPKDSSTW
TVDHQLLWGEALLITPVLQAGKAEVTGYFPLGTWYDLQTVPVEALGSLPPPPAAPREPAIHSEGQWVTLPAPLDTIN
VHLRAGYIIPLQGPGLTTTESRQQPMALAVALTKGGEARGELFWDDGESLEVLERGAYTQVIFLARNNTIVNELVRV
TSEGAGLQLQKVTVLGVATAPQQVLSNGVPVSNFTYSPDTKVLDICVSLLMGEQFLVSWC
(10) 12834B
(SEQ ID NO: 401)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKGGGGSGGGGSGGGGSQVQLVQSGAEVKKPGASVKVSCKASGYTFTS
YGISWVRQAPGQGLEWMGWISVYHGNTNYAQKFQGRVTMTTDTSTSTAYMELRSLRSDDTAVYYCAREGYYDFWSGY
YPFDYWGQGTLVTVSSGGGGSGGGGSAHPGRPRAVPTQCDVPPNSREDCAPDKAITQEQCEARGCCYIPAKQGLQGA
QMGQPWCFFPPSYPSYKLENLSSSEMGYTATLTRTTPTFFPKDILTLRLDVMMETENRLHFTIKDPANRRYEVPLET
PHVHSRAPSPLYSVEFSEEPFGVIVRRQLDGRVLLNTTVAPLFFADQFLQLSTSLPSQYITGLAEHLSPLMLSTSWT
RITLWNRDLAPTPGANLYGSHPFYLALEDGGSAHGVFLLNSNAMDVVLQPSPALSWRSTGGILDVYIFLGPEPKSVV
QQYLDVVGYPFMPPYWGLGFHLCRWGYSSTAITRQVVENMTRAHFPLDVQWNDLDYMDSRRDFTENKDGFRDFPAMV
QELHQGGRRYMMIVDPAISSSGPAGSYRPYDEGLRRGVFITNETGQPLIGKVWPGSTAFPDFTNPTALAWWEDMVAE
FHDQVPFDGMWIDMNEPSNFIRGSEDGCPNNELENPPYVPGVVGGTLQAATICASSHQFLSTHYNLHNLYGLTEAIA
SHRALVKARGTRPFVISRSTFAGHGRYAGHWTGDVWSSWEQLASSVPEILQFNLLGVPLVGADVCGELGNTSEELCV
RWTQLGAFYPFMRNHNSLLSLPQEPYSFSEPAQQAMRKALTLRYALLPHLYTLFHQAHVAGETVARPLFLEFPKDSS
TWTVDHQLLWGEALLITPVLQAGKAEVTGYFPLGTWYDLQTVPVEALGSLPPPPAAPREPAIHSEGQWVTLPAPLDT
INVHLRAGYIIPLQGPGLTTTESRQQPMALAVALTKGGEARGELFWDDGESLEVLERGAYTQVIFLARNNTIVNELV
RVTSEGAGLQLQKVTVLGVATAPQQVLSNGVPVSNFTYSPDTKVLDICVSLLMGEQFLVSWC
(11) 12835B
(SEQ ID NO: 402)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKGGGGSGGGGSGGGGSEVQLVESGGGLIQPGGSLRLSCEASGFTERN
YEMNWVRQAPGKGLEWVSYISSSGNMKDYAESVKGRFTISRDNVKNSLQLQMNSLRVEDTAVYYCARDEFPYGMDVW
GQGTTVTVSSGGGGSGGGGSAHPGRPRAVPTQCDVPPNSREDCAPDKAITQEQCEARGCCYIPAKQGLQGAQMGQPW
CFFPPSYPSYKLENLSSSEMGYTATLTRTTPTFFPKDILTLRLDVMMETENRLHFTIKDPANRRYEVPLETPHVHSR
APSPLYSVEFSEEPFGVIVRRQLDGRVLLNTTVAPLFFADQFLQLSTSLPSQYITGLAEHLSPLMLSTSWTRITLWN
RDLAPTPGANLYGSHPFYLALEDGGSAHGVFLLNSNAMDVVLQPSPALSWRSTGGILDVYIFLGPEPKSVVQQYLDV
VGYPFMPPYWGLGFHLCRWGYSSTAITRQVVENMTRAHFPLDVQWNDLDYMDSRRDFTENKDGERDFPAMVQELHQG
GRRYMMIVDPAISSSGPAGSYRPYDEGLRRGVFITNETGQPLIGKVWPGSTAFPDFTNPTALAWWEDMVAEFHDQVP
FDGMWIDMNEPSNFIRGSEDGCPNNELENPPYVPGVVGGTLQAATICASSHQFLSTHYNLHNLYGLTEAIASHRALV
KARGTRPFVISRSTFAGHGRYAGHWTGDVWSSWEQLASSVPEILQFNLLGVPLVGADVCGFLGNTSEELCVRWTQLG
AFYPFMRNHNSLLSLPQEPYSFSEPAQQAMRKALTLRYALLPHLYTLFHQAHVAGETVARPLFLEFPKDSSTWTVDH
QLLWGEALLITPVLQAGKAEVTGYFPLGTWYDLQTVPVEALGSLPPPPAAPREPAIHSEGQWVTLPAPLDTINVHLR
AGYIIPLQGPGLTTTESRQQPMALAVALTKGGEARGELFWDDGESLEVLERGAYTQVIFLARNNTIVNELVRVTSEG
AGLQLQKVTVLGVATAPQQVLSNGVPVSNFTYSPDTKVLDICVSLLMGEQFLVSWC
(12) 12839B (REGN16822)
(SEQ ID NO: 403)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKGGGGSGGGGSGGGGSQVQLVESGGGVVQPGRSLRLSCAASGFPFSN
YVMYWVRQAPGKGLEWVALIFFDGKKNYHADSVKGRFTITRDNSKNMLYLQMNSLRPEDAAVYYCAKIHCPNGVCYK
GYYGMDVWGQGTTVTVSSGGGGSGGGGSAHPGRPRAVPTQCDVPPNSREDCAPDKAITQEQCEARGCCYIPAKQGLQ
GAQMGQPWCFFPPSYPSYKLENLSSSEMGYTATLTRTTPTFFPKDILTLRLDVMMETENRLHFTIKDPANRRYEVPL
ETPHVHSRAPSPLYSVEFSEEPFGVIVRRQLDGRVLLNTTVAPLFFADQFLQLSTSLPSQYITGLAEHLSPLMLSTS
WTRITLWNRDLAPTPGANLYGSHPFYLALEDGGSAHGVFLLNSNAMDVVLQPSPALSWRSTGGILDVYIFLGPEPKS
VVQQYLDVVGYPFMPPYWGLGFHLCRWGYSSTAITRQVVENMTRAHFPLDVQWNDLDYMDSRRDFTENKDGERDFPA
MVQELHQGGRRYMMIVDPAISSSGPAGSYRPYDEGLRRGVFITNETGQPLIGKVWPGSTAFPDFTNPTALAWWEDMV
AEFHDQVPFDGMWIDMNEPSNFIRGSEDGCPNNELENPPYVPGVVGGTLQAATICASSHQFLSTHYNLHNLYGLTEA
IASHRALVKARGTRPFVISRSTFAGHGRYAGHWTGDVWSSWEQLASSVPEILQFNLLGVPLVGADVCGELGNTSEEL
CVRWTQLGAFYPFMRNHNSLLSLPQEPYSFSEPAQQAMRKALTLRYALLPHLYTLFHQAHVAGETVARPLFLEFPKD
SSTWTVDHQLLWGEALLITPVLQAGKAEVTGYFPLGTWYDLQTVPVEALGSLPPPPAAPREPAIHSEGQWVTLPAPL
DTINVHLRAGYIIPLQGPGLTTTESRQQPMALAVALTKGGEARGELFWDDGESLEVLERGAYTQVIFLARNNTIVNE
LVRVTSEGAGLQLQKVTVLGVATAPQQVLSNGVPVSNFTYSPDTKVLDICVSLLMGEQFLVSWC
(13) 12841B (REGN16823)
(SEQ ID NO: 404)
DIQMTQSPSSLSASVGDRVTITCRASQSISSYLNWYQQKPGKAPKLLIYAASSLQSGVPSRESGSGSGTDFTLTISS
LQPEDFATYYCQQSYSTPPITFGQGTRLEIKGGGGSGGGGSGGGGSEVQLVESGGGLVQPGGSLRLSCAASGFTFSN
YWMNWVRQAPGKGLEWVANIKEDGGKKLYVDSVKGRFTISRDNAKNSLFLQMNSLRAEDTAVYYCAREDTTLVVDYY
YYGMDVWGQGTTVTVSSGGGGSGGGGSAHPGRPRAVPTQCDVPPNSREDCAPDKAITQEQCEARGCCYIPAKQGLQG
AQMGQPWCFFPPSYPSYKLENLSSSEMGYTATLTRTTPTFFPKDILTLRLDVMMETENRLHFTIKDPANRRYEVPLE
TPHVHSRAPSPLYSVEFSEEPFGVIVRRQLDGRVLLNTTVAPLFFADQFLQLSTSLPSQYITGLAEHLSPLMLSTSW
TRITLWNRDLAPTPGANLYGSHPFYLALEDGGSAHGVFLLNSNAMDVVLQPSPALSWRSTGGILDVYIFLGPEPKSV
VQQYLDVVGYPFMPPYWGLGFHLCRWGYSSTAITRQVVENMTRAHFPLDVQWNDLDYMDSRRDFTENKDGFRDFPAM
VQELHQGGRRYMMIVDPAISSSGPAGSYRPYDEGLRRGVFITNETGQPLIGKVWPGSTAFPDFTNPTALAWWEDMVA
EFHDQVPFDGMWIDMNEPSNFIRGSEDGCPNNELENPPYVPGVVGGTLQAATICASSHQFLSTHYNLHNLYGLTEAI
ASHRALVKARGTRPFVISRSTFAGHGRYAGHWTGDVWSSWEQLASSVPEILQFNLLGVPLVGADVCGFLGNTSEELC
VRWTQLGAFYPFMRNHNSLLSLPQEPYSFSEPAQQAMRKALTLRYALLPHLYTLFHQAHVAGETVARPLFLEFPKDS
STWTVDHQLLWGEALLITPVLQAGKAEVTGYFPLGTWYDLQTVPVEALGSLPPPPAAPREPAIHSEGQWVTLPAPLD
TINVHLRAGYIIPLQGPGLTTTESRQQPMALAVALTKGGEARGELFWDDGESLEVLERGAYTQVIFLARNNTIVNEL
VRVTSEGAGLQLQKVTVLGVATAPQQVLSNGVPVSNFTYSPDTKVLDICVSLLMGEQFLVSWC
(14) 12843B (REGN16824)
(SEQ ID NO: 405)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKGGGGSGGGGSGGGGSEVQLVESGGGLVQPGGSLRLSCAASGFTENI
FEMNWVRQAPGKGLEWISYISSRGTTTYYADSVRGRFTISRDNAKNSLYLQMNSLRAEDTAVYYCARDYEATIPFDF
WGQGTLVTVSSGGGGSGGGGSAHPGRPRAVPTQCDVPPNSREDCAPDKAITQEQCEARGCCYIPAKQGLQGAQMGOP
WCFFPPSYPSYKLENLSSSEMGYTATLTRTTPTFFPKDILTLRLDVMMETENRLHFTIKDPANRRYEVPLETPHVHS
RAPSPLYSVEFSEEPFGVIVRRQLDGRVLLNTTVAPLFFADQFLQLSTSLPSQYITGLAEHLSPLMLSTSWTRITLW
NRDLAPTPGANLYGSHPFYLALEDGGSAHGVFLLNSNAMDVVLQPSPALSWRSTGGILDVYIFLGPEPKSVVQQYLD
VVGYPFMPPYWGLGFHLCRWGYSSTAITRQVVENMTRAHFPLDVQWNDLDYMDSRRDFTENKDGFRDFPAMVQELHQ
GGRRYMMIVDPAISSSGPAGSYRPYDEGLRRGVFITNETGQPLIGKVWPGSTAFPDFTNPTALAWWEDMVAEFHDQV
PFDGMWIDMNEPSNFIRGSEDGCPNNELENPPYVPGVVGGTLQAATICASSHQFLSTHYNLHNLYGLTEAIASHRAL
VKARGTRPFVISRSTFAGHGRYAGHWTGDVWSSWEQLASSVPEILQFNLLGVPLVGADVCGFLGNTSEELCVRWTQL
GAFYPFMRNHNSLLSLPQEPYSFSEPAQQAMRKALTLRYALLPHLYTLFHQAHVAGETVARPLFLEFPKDSSTWTVD
HQLLWGEALLITPVLQAGKAEVTGYFPLGTWYDLQTVPVEALGSLPPPPAAPREPAIHSEGQWVTLPAPLDTINVHL
RAGYIIPLQGPGLTTTESRQQPMALAVALTKGGEARGELFWDDGESLEVLERGAYTQVIFLARNNTIVNELVRVTSE
GAGLQLQKVTVLGVATAPQQVLSNGVPVSNFTYSPDTKVLDICVSLLMGEQFLVSWC
(15) 12844B
(SEQ ID NO: 406)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKGGGGSGGGGSGGGGSEVQLVESGGSVVRPGGSLRLSCEASGFTEDD
YGMSWVRQDPGKGLEWVSGINWNGDRTNYADSVKGRFIISRDNAKNSVYLQMNSLRAEDSALYHCARDQGLGVAATL
DYWGQGTLVTVSSGGGGSGGGGSAHPGRPRAVPTQCDVPPNSRFDCAPDKAITQEQCEARGCCYIPAKQGLQGAQMG
QPWCFFPPSYPSYKLENLSSSEMGYTATLTRTTPTFFPKDILTLRLDVMMETENRLHFTIKDPANRRYEVPLETPHV
HSRAPSPLYSVEFSEEPFGVIVRRQLDGRVLLNTTVAPLFFADQFLQLSTSLPSQYITGLAEHLSPLMLSTSWTRIT
LWNRDLAPTPGANLYGSHPFYLALEDGGSAHGVFLLNSNAMDVVLQPSPALSWRSTGGILDVYIFLGPEPKSVVQQY
LDVVGYPFMPPYWGLGFHLCRWGYSSTAITRQVVENMTRAHFPLDVQWNDLDYMDSRRDFTENKDGERDFPAMVQEL
HQGGRRYMMIVDPAISSSGPAGSYRPYDEGLRRGVFITNETGQPLIGKVWPGSTAFPDFTNPTALAWWEDMVAEFHD
QVPFDGMWIDMNEPSNFIRGSEDGCPNNELENPPYVPGVVGGTLQAATICASSHQFLSTHYNLHNLYGLTEAIASHR
ALVKARGTRPFVISRSTFAGHGRYAGHWTGDVWSSWEQLASSVPEILQFNLLGVPLVGADVCGFLGNTSEELCVRWT
QLGAFYPFMRNHNSLLSLPQEPYSFSEPAQQAMRKALTLRYALLPHLYTLFHQAHVAGETVARPLFLEFPKDSSTWT
VDHQLLWGEALLITPVLQAGKAEVTGYFPLGTWYDLQTVPVEALGSLPPPPAAPREPAIHSEGQWVTLPAPLDTINV
HLRAGYIIPLQGPGLTTTESRQQPMALAVALTKGGEARGELFWDDGESLEVLERGAYTQVIFLARNNTIVNELVRVT
SEGAGLQLQKVTVLGVATAPQQVLSNGVPVSNFTYSPDTKVLDICVSLLMGEQFLVSWC
(16) 12845B (REGN16825)
(SEQ ID NO: 407)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKGGGGSGGGGSGGGGSEVQLVESGGGLVQPGGSLRLSCAASGFTVSN
YEMNWVRQAPGKGLEWVSYISSSTSNIYYADSVKGRFTISRDNAENSLYLQMNSLRVEDTAVYYCVRDGIVVVPVGR
GYYYYGLDVWGQGTTVTVSSGGGGSGGGGSAHPGRPRAVPTQCDVPPNSREDCAPDKAITQEQCEARGCCYIPAKQG
LQGAQMGQPWCFFPPSYPSYKLENLSSSEMGYTATLTRTTPTFFPKDILTLRLDVMMETENRLHFTIKDPANRRYEV
PLETPHVHSRAPSPLYSVEFSEEPFGVIVRRQLDGRVLLNTTVAPLFFADQFLQLSTSLPSQYITGLAEHLSPLMLS
TSWTRITLWNRDLAPTPGANLYGSHPFYLALEDGGSAHGVFLLNSNAMDVVLQPSPALSWRSTGGILDVYIFLGPEP
KSVVQQYLDVVGYPFMPPYWGLGFHLCRWGYSSTAITRQVVENMTRAHFPLDVQWNDLDYMDSRRDFTENKDGERDF
PAMVQELHQGGRRYMMIVDPAISSSGPAGSYRPYDEGLRRGVFITNETGQPLIGKVWPGSTAFPDFTNPTALAWWED
MVAEFHDQVPFDGMWIDMNEPSNFIRGSEDGCPNNELENPPYVPGVVGGTLQAATICASSHQFLSTHYNLHNLYGLT
EAIASHRALVKARGTRPFVISRSTFAGHGRYAGHWTGDVWSSWEQLASSVPEILQFNLLGVPLVGADVCGELGNTSE
ELCVRWTQLGAFYPFMRNHNSLLSLPQEPYSFSEPAQQAMRKALTLRYALLPHLYTLFHQAHVAGETVARPLFLEFP
KDSSTWTVDHQLLWGEALLITPVLQAGKAEVTGYFPLGTWYDLQTVPVEALGSLPPPPAAPREPAIHSEGQWVTLPA
PLDTINVHLRAGYIIPLQGPGLTTTESRQQPMALAVALTKGGEARGELFWDDGESLEVLERGAYTQVIFLARNNTIV
NELVRVTSEGAGLQLQKVTVLGVATAPQQVLSNGVPVSNFTYSPDTKVLDICVSLLMGEQFLVSWC
(17) 12847B (REGN16826)
(SEQ ID NO: 408)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKGGGGSGGGGSGGGGSEVQLVESGGGLVQPGRSLRLSCAASGFTFDD
YAMNWVRQAPGKGLEWVSGISWSSGSMDYADSVKGRFTISRDNAKNSLYLQMNSLRTEDTALYYCAKAREVGDYYGM
DVWGQGTTVTVSSGGGGSGGGGSAHPGRPRAVPTQCDVPPNSREDCAPDKAITQEQCEARGCCYIPAKQGLQGAQMG
QPWCFFPPSYPSYKLENLSSSEMGYTATLTRTTPTFFPKDILTLRLDVMMETENRLHFTIKDPANRRYEVPLETPHV
HSRAPSPLYSVEFSEEPFGVIVRRQLDGRVLLNTTVAPLFFADQFLQLSTSLPSQYITGLAEHLSPLMLSTSWTRIT
LWNRDLAPTPGANLYGSHPFYLALEDGGSAHGVFLLNSNAMDVVLQPSPALSWRSTGGILDVYIFLGPEPKSVVQQY
LDVVGYPFMPPYWGLGFHLCRWGYSSTAITRQVVENMTRAHFPLDVQWNDLDYMDSRRDFTENKDGERDFPAMVQEL
HQGGRRYMMIVDPAISSSGPAGSYRPYDEGLRRGVFITNETGQPLIGKVWPGSTAFPDFTNPTALAWWEDMVAEFHD
QVPFDGMWIDMNEPSNFIRGSEDGCPNNELENPPYVPGVVGGTLQAATICASSHQFLSTHYNLHNLYGLTEAIASHR
ALVKARGTRPFVISRSTFAGHGRYAGHWTGDVWSSWEQLASSVPEILQFNLLGVPLVGADVCGFLGNTSEELCVRWT
QLGAFYPFMRNHNSLLSLPQEPYSFSEPAQQAMRKALTLRYALLPHLYTLFHQAHVAGETVARPLFLEFPKDSSTWT
VDHQLLWGEALLITPVLQAGKAEVTGYFPLGTWYDLQTVPVEALGSLPPPPAAPREPAIHSEGQWVTLPAPLDTINV
HLRAGYIIPLQGPGLTTTESRQQPMALAVALTKGGEARGELFWDDGESLEVLERGAYTQVIFLARNNTIVNELVRVT
SEGAGLQLQKVTVLGVATAPQQVLSNGVPVSNFTYSPDTKVLDICVSLLMGEQFLVSWC
(18) 12848B (REGN16827)
(SEQ ID NO: 409)
EIVLTQSPGTLSLSPGERATLSCRASQSVSSSYLAWYQQKPGQAPRLLIYGASSRATGIPDRESGSGSGTDFTLTIS
RLEPEDFAVYYCQQYGSSPWTFGQGTKVEIKGGGGSGGGGSGGGGSEVQLVESGGGLVQPGRSLTLSCAASGFTFDN
FGMHWVRQGPGKGLEWVSGLTWNSGVIGYADSVKGRFTISRDNAKNSLYLQMNSLRPEDTALYYCAKDIRNYGPFDY
WGQGTLVTVSSGGGGSGGGGSAHPGRPRAVPTQCDVPPNSREDCAPDKAITQEQCEARGCCYIPAKQGLQGAQMGQP
WCFFPPSYPSYKLENLSSSEMGYTATLTRTTPTFFPKDILTLRLDVMMETENRLHFTIKDPANRRYEVPLETPHVHS
RAPSPLYSVEFSEEPFGVIVRRQLDGRVLLNTTVAPLFFADQFLQLSTSLPSQYITGLAEHLSPLMLSTSWTRITLW
NRDLAPTPGANLYGSHPFYLALEDGGSAHGVFLLNSNAMDVVLQPSPALSWRSTGGILDVYIFLGPEPKSVVQQYLD
VVGYPFMPPYWGLGFHLCRWGYSSTAITRQVVENMTRAHFPLDVQWNDLDYMDSRRDFTENKDGERDFPAMVQELHQ
GGRRYMMIVDPAISSSGPAGSYRPYDEGLRRGVFITNETGQPLIGKVWPGSTAFPDFTNPTALAWWEDMVAEFHDQV
PFDGMWIDMNEPSNFIRGSEDGCPNNELENPPYVPGVVGGTLQAATICASSHQFLSTHYNLHNLYGLTEAIASHRAL
VKARGTRPFVISRSTFAGHGRYAGHWTGDVWSSWEQLASSVPEILQFNLLGVPLVGADVCGFLGNTSEELCVRWTQL
GAFYPFMRNHNSLLSLPQEPYSFSEPAQQAMRKALTLRYALLPHLYTLFHQAHVAGETVARPLFLEFPKDSSTWTVD
HQLLWGEALLITPVLQAGKAEVTGYFPLGTWYDLQTVPVEALGSLPPPPAAPREPAIHSEGQWVTLPAPLDTINVHL
RAGYIIPLQGPGLTTTESRQQPMALAVALTKGGEARGELFWDDGESLEVLERGAYTQVIFLARNNTIVNELVRVTSE
GAGLQLQKVTVLGVATAPQQVLSNGVPVSNFTYSPDTKVLDICVSLLMGEQFLVSWC
(19) 12850B (REGN16828)
(SEQ ID NO: 410)
EIVLTQSPGTLSLSPGERATLSCRASQSVSSSYLAWYQQKPGQAPRLLIYGASSRATGIPDRFSGSGSGTDFTLTIS
RLEPEDFAVYYCQQYGSSPWTFGQGTKVEIKGGGGSGGGGSGGGGSQVQLVQSGAEVKKPGSSVKVSCKASGGTENT
YAITWVRQAPGQGLEWMGGIIPISGIAEYAQKFQGRVTITTDDSSTTAYMELNSLRSEDTAVYYCASWNYALYYFYG
MDVWGRGTTVTVSSGGGGSGGGGSAHPGRPRAVPTQCDVPPNSREDCAPDKAITQEQCEARGCCYIPAKQGLQGAQM
GQPWCFFPPSYPSYKLENLSSSEMGYTATLTRTTPTFFPKDILTLRLDVMMETENRLHFTIKDPANRRYEVPLETPH
VHSRAPSPLYSVEFSEEPFGVIVRRQLDGRVLLNTTVAPLFFADQFLQLSTSLPSQYITGLAEHLSPLMLSTSWTRI
TLWNRDLAPTPGANLYGSHPFYLALEDGGSAHGVFLLNSNAMDVVLQPSPALSWRSTGGILDVYIFLGPEPKSVVQQ
YLDVVGYPFMPPYWGLGFHLCRWGYSSTAITRQVVENMTRAHFPLDVQWNDLDYMDSRRDFTENKDGERDFPAMVQE
LHQGGRRYMMIVDPAISSSGPAGSYRPYDEGLRRGVFITNETGQPLIGKVWPGSTAFPDFTNPTALAWWEDMVAEFH
DQVPFDGMWIDMNEPSNFIRGSEDGCPNNELENPPYVPGVVGGTLQAATICASSHQFLSTHYNLHNLYGLTEAIASH
RALVKARGTRPFVISRSTFAGHGRYAGHWTGDVWSSWEQLASSVPEILQFNLLGVPLVGADVCGFLGNTSEELCVRW
TQLGAFYPFMRNHNSLLSLPQEPYSFSEPAQQAMRKALTLRYALLPHLYTLFHQAHVAGETVARPLFLEFPKDSSTW
TVDHQLLWGEALLITPVLQAGKAEVTGYFPLGTWYDLQTVPVEALGSLPPPPAAPREPAIHSEGQWVTLPAPLDTIN
VHLRAGYIIPLQGPGLTTTESRQQPMALAVALTKGGEARGELFWDDGESLEVLERGAYTQVIFLARNNTIVNELVRV
TSEGAGLQLQKVTVLGVATAPQQVLSNGVPVSNFTYSPDTKVLDICVSLLMGEQFLVSWC
(20) 31863B
(SEQ ID NO: 411)
DIQMTQSPSSLSASIGDRVTITCRASQGISNYLAWYQQKPGKVPKLLIYAASTLQSGVPSRFSGSGSGTDFTLTISS
LQPEDVATYYCQNHNSVPLTFGGGTKVEIKGGGGSGGGGSGGGGSEVQLVESGGGLVQPGGSLRLSCAASGFTENSY
AMTWVRQAPGKGLEWVSFIGGSTGNTYYAGSVKGRFTISSDNSKKTLYLQMNSLRAEDTAVYYCAKGGAARRMEYFQ
HWGQGTLVTVSSGGGGSGGGGSAHPGRPRAVPTQCDVPPNSREDCAPDKAITQEQCEARGCCYIPAKQGLQGAQMGQ
PWCFFPPSYPSYKLENLSSSEMGYTATLTRTTPTFFPKDILTLRLDVMMETENRLHFTIKDPANRRYEVPLETPHVH
SRAPSPLYSVEFSEEPFGVIVRRQLDGRVLLNTTVAPLFFADQFLQLSTSLPSQYITGLAEHLSPLMLSTSWTRITL
WNRDLAPTPGANLYGSHPFYLALEDGGSAHGVFLLNSNAMDVVLQPSPALSWRSTGGILDVYIFLGPEPKSVVQQYL
DVVGYPFMPPYWGLGFHLCRWGYSSTAITRQVVENMTRAHFPLDVQWNDLDYMDSRRDFTENKDGERDFPAMVQELH
QGGRRYMMIVDPAISSSGPAGSYRPYDEGLRRGVFITNETGQPLIGKVWPGSTAFPDFTNPTALAWWEDMVAEFHDQ
VPFDGMWIDMNEPSNFIRGSEDGCPNNELENPPYVPGVVGGTLQAATICASSHQFLSTHYNLHNLYGLTEAIASHRA
LVKARGTRPFVISRSTFAGHGRYAGHWTGDVWSSWEQLASSVPEILQFNLLGVPLVGADVCGFLGNTSEELCVRWTQ
LGAFYPFMRNHNSLLSLPQEPYSFSEPAQQAMRKALTLRYALLPHLYTLFHQAHVAGETVARPLFLEFPKDSSTWTV
DHQLLWGEALLITPVLQAGKAEVTGYFPLGTWYDLQTVPVEALGSLPPPPAAPREPAIHSEGQWVTLPAPLDTINVH
LRAGYIIPLQGPGLTTTESRQQPMALAVALTKGGEARGELFWDDGESLEVLERGAYTQVIFLARNNTIVNELVRVTS
EGAGLQLQKVTVLGVATAPQQVLSNGVPVSNFTYSPDTKVLDICVSLLMGEQFLVSWC
(21) 31874B
(SEQ ID NO: 412)
DIQMTQSPSSLSASVGDRVTITCRASQGISNYLAWYQQKPGKVPNLLIYAASTLQSGVPSRESGSGSGTDFTLTISS
LQPEDVATYYCQKYNSAPLTFGGGTKVEIKGGGGSGGGGSGGGGSEVQLVESGGGLVQPGGSLRLSCAASGFAFSSY
AMTWVRQAPGKGLEWVSVISGTGGSTYYADSVKGRFTISRDNSKNTLYLQMNSLRAEDTAVYYCAKGGAARRMEYFQ
YWGQGTLVTVSSGGGGSGGGGSAHPGRPRAVPTQCDVPPNSREDCAPDKAITQEQCEARGCCYIPAKQGLQGAQMGQ
PWCFFPPSYPSYKLENLSSSEMGYTATLTRTTPTFFPKDILTLRLDVMMETENRLHFTIKDPANRRYEVPLETPHVH
SRAPSPLYSVEFSEEPFGVIVRRQLDGRVLLNTTVAPLFFADQFLQLSTSLPSQYITGLAEHLSPLMLSTSWTRITL
WNRDLAPTPGANLYGSHPFYLALEDGGSAHGVFLLNSNAMDVVLQPSPALSWRSTGGILDVYIFLGPEPKSVVQQYL
DVVGYPFMPPYWGLGFHLCRWGYSSTAITRQVVENMTRAHFPLDVQWNDLDYMDSRRDFTENKDGFRDFPAMVQELH
QGGRRYMMIVDPAISSSGPAGSYRPYDEGLRRGVFITNETGQPLIGKVWPGSTAFPDFTNPTALAWWEDMVAEFHDQ
VPFDGMWIDMNEPSNFIRGSEDGCPNNELENPPYVPGVVGGTLQAATICASSHQFLSTHYNLHNLYGLTEAIASHRA
LVKARGTRPFVISRSTFAGHGRYAGHWTGDVWSSWEQLASSVPEILQFNLLGVPLVGADVCGFLGNTSEELCVRWTQ
LGAFYPFMRNHNSLLSLPQEPYSFSEPAQQAMRKALTLRYALLPHLYTLFHQAHVAGETVARPLFLEFPKDSSTWTV
DHQLLWGEALLITPVLQAGKAEVTGYFPLGTWYDLQTVPVEALGSLPPPPAAPREPAIHSEGQWVTLPAPLDTINVH
LRAGYIIPLQGPGLTTTESRQQPMALAVALTKGGEARGELFWDDGESLEVLERGAYTQVIFLARNNTIVNELVRVTS
EGAGLQLQKVTVLGVATAPQQVLSNGVPVSNFTYSPDTKVLDICVSLLMGEQFLVSWC
(22) 69261
(SEQ ID NO: 413)
DIVMTQSPLSLPVTPGEPASISCRSSQSLLHSNGYNYLDWYLQKPGQSPQFLIYLGSNRASGVPDRESGSGSGTDFT
LKINRVEAEDVGVYYCMQALQTPYTFGQGTKLEIKGGGGSGGGGSGGGGSQVQLVESGGGLVKPGGSLRLSCAASGF
TFSVYYMNWIRQAPGKGLEWVSYISSSGSTIYYADSVKGRFTISRDNAKNSLYLQMNSLRAEDTAVYYCGREGYSGT
YSYYGMDVWGQGTTVTVSSGGGGSGGGGSAHPGRPRAVPTQCDVPPNSRFDCAPDKAITQEQCEARGCCYIPAKQGL
QGAQMGQPWCFFPPSYPSYKLENLSSSEMGYTATLTRTTPTFFPKDILTLRLDVMMETENRLHFTIKDPANRRYEVP
LETPHVHSRAPSPLYSVEFSEEPFGVIVRRQLDGRVLLNTTVAPLFFADQFLQLSTSLPSQYITGLAEHLSPLMLST
SWTRITLWNRDLAPTPGANLYGSHPFYLALEDGGSAHGVFLLNSNAMDVVLQPSPALSWRSTGGILDVYIFLGPEPK
SVVQQYLDVVGYPFMPPYWGLGFHLCRWGYSSTAITRQVVENMTRAHFPLDVQWNDLDYMDSRRDFTENKDGERDFP
AMVQELHQGGRRYMMIVDPAISSSGPAGSYRPYDEGLRRGVFITNETGQPLIGKVWPGSTAFPDFTNPTALAWWEDM
VAEFHDQVPFDGMWIDMNEPSNFIRGSEDGCPNNELENPPYVPGVVGGTLQAATICASSHQFLSTHYNLHNLYGLTE
AIASHRALVKARGTRPFVISRSTFAGHGRYAGHWTGDVWSSWEQLASSVPEILQFNLLGVPLVGADVCGELGNTSEE
LCVRWTQLGAFYPFMRNHNSLLSLPQEPYSFSEPAQQAMRKALTLRYALLPHLYTLFHQAHVAGETVARPLFLEFPK
DSSTWTVDHQLLWGEALLITPVLQAGKAEVTGYFPLGTWYDLQTVPVEALGSLPPPPAAPREPAIHSEGQWVTLPAP
LDTINVHLRAGYIIPLQGPGLTTTESRQQPMALAVALTKGGEARGELFWDDGESLEVLERGAYTQVIFLARNNTIVN
ELVRVTSEGAGLQLQKVTVLGVATAPQQVLSNGVPVSNFTYSPDTKVLDICVSLLMGEQFLVSWC
(23) 69263
(SEQ ID NO: 414)
DIQMTQSPSSLSASVGDRVTITCRASQDISHYSAWYQQKPGKLPNLLIYAASTLQSGVPSRFSGSGSGTDESLTTSS
LQPEDVATYYCQKYNSVPLTFGGGTKVEIKGGGGSGGGGSGGGGSEVQLVESGGGLVQPGRSLRLSCAVSGFTEDDY
AMHWVRQAPGKGLEWVSGISWNSGTRGYADSVKGRFTISRDNAKNSLYLQMNSLRGEDTALYYCVKDITISPNYYGM
DVWGQGTTVTVSSGGGGSGGGGSAHPGRPRAVPTQCDVPPNSREDCAPDKAITQEQCEARGCCYIPAKQGLQGAQMG
QPWCFFPPSYPSYKLENLSSSEMGYTATLTRTTPTFFPKDILTLRLDVMMETENRLHFTIKDPANRRYEVPLETPHV
HSRAPSPLYSVEFSEEPFGVIVRRQLDGRVLLNTTVAPLFFADQFLQLSTSLPSQYITGLAEHLSPLMLSTSWTRIT
LWNRDLAPTPGANLYGSHPFYLALEDGGSAHGVFLLNSNAMDVVLQPSPALSWRSTGGILDVYIFLGPEPKSVVQQY
LDVVGYPFMPPYWGLGFHLCRWGYSSTAITRQVVENMTRAHFPLDVQWNDLDYMDSRRDFTENKDGFRDFPAMVQEL
HQGGRRYMMIVDPAISSSGPAGSYRPYDEGLRRGVFITNETGQPLIGKVWPGSTAFPDFTNPTALAWWEDMVAEFHD
QVPFDGMWIDMNEPSNFIRGSEDGCPNNELENPPYVPGVVGGTLQAATICASSHQFLSTHYNLHNLYGLTEAIASHR
ALVKARGTRPFVISRSTFAGHGRYAGHWTGDVWSSWEQLASSVPEILQFNLLGVPLVGADVCGELGNTSEELCVRWT
QLGAFYPFMRNHNSLLSLPQEPYSFSEPAQQAMRKALTLRYALLPHLYTLFHQAHVAGETVARPLFLEFPKDSSTWT
VDHQLLWGEALLITPVLQAGKAEVTGYFPLGTWYDLQTVPVEALGSLPPPPAAPREPAIHSEGQWVTLPAPLDTINV
HLRAGYIIPLQGPGLTTTESRQQPMALAVALTKGGEARGELFWDDGESLEVLERGAYTQVIFLARNNTIVNELVRVT
SEGAGLQLQKVTVLGVATAPQQVLSNGVPVSNFTYSPDTKVLDICVSLLMGEQFLVSWC
(24) 69305
(SEQ ID NO: 415)
DIQMTQSPSSLSASVGDRVTITCRASQSIDRYLNWYRQKPGKAPKLLIYTTSSLQSGVPSRFSGSGSGTDFTLTLSS
LQPEDFATYYCQQSYSPPLTFGGGTKVEIKGGGGSGGGGSGGGGSQVQLVESGGGVVQPGRSLRLSCAASGFTFSSY
GMHWVRQAPGKGLEWVAVIWYDGSNKYYADSVKGRFTISRDISKNTLYLQMNSLRAEDTAVYYCAGQLDLFFDYWGQ
GTLVTVSSGGGGSGGGGSAHPGRPRAVPTQCDVPPNSREDCAPDKAITQEQCEARGCCYIPAKQGLQGAQMGQPWCF
FPPSYPSYKLENLSSSEMGYTATLTRTTPTFFPKDILTLRLDVMMETENRLHFTIKDPANRRYEVPLETPHVHSRAP
SPLYSVEFSEEPFGVIVRRQLDGRVLLNTTVAPLFFADQFLQLSTSLPSQYITGLAEHLSPLMLSTSWTRITLWNRD
LAPTPGANLYGSHPFYLALEDGGSAHGVFLLNSNAMDVVLQPSPALSWRSTGGILDVYIFLGPEPKSVVQQYLDVVG
YPFMPPYWGLGFHLCRWGYSSTAITRQVVENMTRAHFPLDVQWNDLDYMDSRRDFTENKDGERDFPAMVQELHOGGR
RYMMIVDPAISSSGPAGSYRPYDEGLRRGVFITNETGQPLIGKVWPGSTAFPDFTNPTALAWWEDMVAEFHDQVPFD
GMWIDMNEPSNFIRGSEDGCPNNELENPPYVPGVVGGTLQAATICASSHQFLSTHYNLHNLYGLTEAIASHRALVKA
RGTRPFVISRSTFAGHGRYAGHWTGDVWSSWEQLASSVPEILQFNLLGVPLVGADVCGFLGNTSEELCVRWTQLGAF
YPFMRNHNSLLSLPQEPYSFSEPAQQAMRKALTLRYALLPHLYTLFHQAHVAGETVARPLFLEFPKDSSTWTVDHQL
LWGEALLITPVLQAGKAEVTGYFPLGTWYDLQTVPVEALGSLPPPPAAPREPAIHSEGQWVTLPAPLDTINVHLRAG
YIIPLQGPGLTTTESRQQPMALAVALTKGGEARGELFWDDGESLEVLERGAYTQVIFLARNNTIVNELVRVTSEGAG
LQLQKVTVLGVATAPQQVLSNGVPVSNFTYSPDTKVLDICVSLLMGEQFLVSWC
(25) 69307 (REGN16817)
(SEQ ID NO: 416)
DIQMTQSPSSVSASVGDRVTITCRASQGISSWLAWYQQKPGKAPKLLIYAASSLQSGVPSRFSGSGSGTDFTLTISS
LQPEDFATYYCQKADSLPYAFGQGTKLEIKGGGGSGGGGSGGGGSEVQLVESGGGLVQPGGSLRLSCTASGFTFSNY
WMTWVRQAPGKGLEWVANIKEDGSEKEYVDSVKGRFTISRDNAKNSLYLQMNSLRGEDTAVYYCARDGEQLVDYYYY
YVMDVWGQGTTVTVSSGGGGSGGGGSAHPGRPRAVPTQCDVPPNSREDCAPDKAITQEQCEARGCCYIPAKQGLQGA
QMGQPWCFFPPSYPSYKLENLSSSEMGYTATLTRTTPTFFPKDILTLRLDVMMETENRLHFTIKDPANRRYEVPLET
PHVHSRAPSPLYSVEFSEEPFGVIVRRQLDGRVLLNTTVAPLFFADQFLQLSTSLPSQYITGLAEHLSPLMLSTSWT
RITLWNRDLAPTPGANLYGSHPFYLALEDGGSAHGVFLLNSNAMDVVLQPSPALSWRSTGGILDVYIFLGPEPKSVV
QQYLDVVGYPFMPPYWGLGFHLCRWGYSSTAITRQVVENMTRAHFPLDVQWNDLDYMDSRRDFTENKDGERDFPAMV
QELHQGGRRYMMIVDPAISSSGPAGSYRPYDEGLRRGVFITNETGQPLIGKVWPGSTAFPDFTNPTALAWWEDMVAE
FHDQVPFDGMWIDMNEPSNFIRGSEDGCPNNELENPPYVPGVVGGTLQAATICASSHQFLSTHYNLHNLYGLTEAIA
SHRALVKARGTRPFVISRSTFAGHGRYAGHWTGDVWSSWEQLASSVPEILQFNLLGVPLVGADVCGFLGNTSEELCV
RWTQLGAFYPFMRNHNSLLSLPQEPYSFSEPAQQAMRKALTLRYALLPHLYTLFHQAHVAGETVARPLFLEFPKDSS
TWTVDHQLLWGEALLITPVLQAGKAEVTGYFPLGTWYDLQTVPVEALGSLPPPPAAPREPAIHSEGQWVTLPAPLDT
INVHLRAGYIIPLQGPGLTTTESRQQPMALAVALTKGGEARGELFWDDGESLEVLERGAYTQVIFLARNNTIVNELV
RVTSEGAGLQLQKVTVLGVATAPQQVLSNGVPVSNFTYSPDTKVLDICVSLLMGEQFLVSWC
(26) 69323 (REGN16816)
(SEQ ID NO: 417)
DIQMTQSPSSLSASVGDRVTITCRASQSISSYLNWYQQKPGKAPKVLIYAASSLQSGVPSRFSGSGSGTDFTLTISS
LQPEDFATYYCQQSYSIPLTFGGGTKVEIKGGGGSGGGGSGGGGSEVQLVESGGGLVQPGRSLRLSCAASGFTEDDY
AMHWVRQAPGKGLEWVSGISWNSGYIGYADSVKGRFTISRDNAENSLHLQMNSLRAEDTALYYCARGGSTLVRGVKG
GYYGMDVWGQGTTVTVSSGGGGSGGGGSAHPGRPRAVPTQCDVPPNSREDCAPDKAITQEQCEARGCCYIPAKQGLQ
GAQMGQPWCFFPPSYPSYKLENLSSSEMGYTATLTRTTPTFFPKDILTLRLDVMMETENRLHFTIKDPANRRYEVPL
ETPHVHSRAPSPLYSVEFSEEPFGVIVRRQLDGRVLLNTTVAPLFFADQFLQLSTSLPSQYITGLAEHLSPLMLSTS
WTRITLWNRDLAPTPGANLYGSHPFYLALEDGGSAHGVFLLNSNAMDVVLQPSPALSWRSTGGILDVYIFLGPEPKS
VVQQYLDVVGYPFMPPYWGLGFHLCRWGYSSTAITRQVVENMTRAHFPLDVQWNDLDYMDSRRDFTENKDGERDFPA
MVQELHQGGRRYMMIVDPAISSSGPAGSYRPYDEGLRRGVFITNETGQPLIGKVWPGSTAFPDFTNPTALAWWEDMV
AEFHDQVPFDGMWIDMNEPSNFIRGSEDGCPNNELENPPYVPGVVGGTLQAATICASSHQFLSTHYNLHNLYGLTEA
IASHRALVKARGTRPFVISRSTFAGHGRYAGHWTGDVWSSWEQLASSVPEILQFNLLGVPLVGADVCGELGNTSEEL
CVRWTQLGAFYPFMRNHNSLLSLPQEPYSFSEPAQQAMRKALTLRYALLPHLYTLFHQAHVAGETVARPLFLEFPKD
SSTWTVDHQLLWGEALLITPVLQAGKAEVTGYFPLGTWYDLQTVPVEALGSLPPPPAAPREPAIHSEGQWVTLPAPL
DTINVHLRAGYIIPLQGPGLTTTESRQQPMALAVALTKGGEARGELFWDDGESLEVLERGAYTQVIFLARNNTIVNE
LVRVTSEGAGLQLQKVTVLGVATAPQQVLSNGVPVSNFTYSPDTKVLDICVSLLMGEQFLVSWC
(27) 69326
(SEQ ID NO: 418)
EIVMTQSPATLSVSPGERATLSCRASQSVSSNFAWYQQKPGQAPRLLIYSASSRATGIPVRFSGSGSGTEFTLTISS
LQSEDFAVYYCQQYNIWPRTFGQGTKVEIKGGGGSGGGGSGGGGSEVQLVESGGGLVQPGGSLRLSCAVSGFIFSSY
EMNWVRQAPGKGLEWVSYISSSGSTIFYADSVKGRFTISRDNAKNSLYLQMNSLRAEDTAVYYCVSGVVLFDVWGQG
TMVTVSSGGGGSGGGGSAHPGRPRAVPTQCDVPPNSREDCAPDKAITQEQCEARGCCYIPAKQGLQGAQMGQPWCFF
PPSYPSYKLENLSSSEMGYTATLTRTTPTFFPKDILTLRLDVMMETENRLHFTIKDPANRRYEVPLETPHVHSRAPS
PLYSVEFSEEPFGVIVRRQLDGRVLLNTTVAPLFFADQFLQLSTSLPSQYITGLAEHLSPLMLSTSWTRITLWNRDL
APTPGANLYGSHPFYLALEDGGSAHGVFLLNSNAMDVVLQPSPALSWRSTGGILDVYIFLGPEPKSVVQQYLDVVGY
PFMPPYWGLGFHLCRWGYSSTAITRQVVENMTRAHFPLDVQWNDLDYMDSRRDFTENKDGERDFPAMVQELHQGGRR
YMMIVDPAISSSGPAGSYRPYDEGLRRGVFITNETGQPLIGKVWPGSTAFPDFTNPTALAWWEDMVAEFHDQVPFDG
MWIDMNEPSNFIRGSEDGCPNNELENPPYVPGVVGGTLQAATICASSHQFLSTHYNLHNLYGLTEAIASHRALVKAR
GTRPFVISRSTFAGHGRYAGHWTGDVWSSWEQLASSVPEILQFNLLGVPLVGADVCGFLGNTSEELCVRWTQLGAFY
PFMRNHNSLLSLPQEPYSFSEPAQQAMRKALTLRYALLPHLYTLFHQAHVAGETVARPLFLEFPKDSSTWTVDHQLL
WGEALLITPVLQAGKAEVTGYFPLGTWYDLQTVPVEALGSLPPPPAAPREPAIHSEGQWVTLPAPLDTINVHLRAGY
IIPLQGPGLTTTESRQQPMALAVALTKGGEARGELFWDDGESLEVLERGAYTQVIFLARNNTIVNELVRVTSEGAGL
QLQKVTVLGVATAPQQVLSNGVPVSNFTYSPDTKVLDICVSLLMGEQFLVSWC
(28) 69329
(SEQ ID NO: 419)
DIQMTQSPSSVSASVGDRVTITCRASQGISSWLAWYQQKPGKAPKLLIYAASSLQSGVPSRFSGSGSGTDFTLTISS
LQPEDFATYYCQKANSFPYTFGQGTKLEIKGGGGSGGGGSGGGGSEVQLVESGGGLVQPGGSLRLSCAASGFTFSNY
WMTWVRQAPGKGLEWVANIKEDGSEKDYVDSVKGRFTISRDNAKNSLYLQMNSLRGEDTAVYYCARDGEQLVDYYYY
YVMDVWGQGTTVTVSSGGGGSGGGGSAHPGRPRAVPTQCDVPPNSREDCAPDKAITQEQCEARGCCYIPAKQGLQGA
QMGQPWCFFPPSYPSYKLENLSSSEMGYTATLTRTTPTFFPKDILTLRLDVMMETENRLHFTIKDPANRRYEVPLET
PHVHSRAPSPLYSVEFSEEPFGVIVRRQLDGRVLLNTTVAPLFFADQFLQLSTSLPSQYITGLAEHLSPLMLSTSWT
RITLWNRDLAPTPGANLYGSHPFYLALEDGGSAHGVFLLNSNAMDVVLQPSPALSWRSTGGILDVYIFLGPEPKSVV
QQYLDVVGYPFMPPYWGLGFHLCRWGYSSTAITRQVVENMTRAHFPLDVQWNDLDYMDSRRDFTENKDGERDFPAMV
QELHQGGRRYMMIVDPAISSSGPAGSYRPYDEGLRRGVFITNETGQPLIGKVWPGSTAFPDFTNPTALAWWEDMVAE
FHDQVPFDGMWIDMNEPSNFIRGSEDGCPNNELENPPYVPGVVGGTLQAATICASSHQFLSTHYNLHNLYGLTEAIA
SHRALVKARGTRPFVISRSTFAGHGRYAGHWTGDVWSSWEQLASSVPEILQFNLLGVPLVGADVCGFLGNTSEELCV
RWTQLGAFYPFMRNHNSLLSLPQEPYSFSEPAQQAMRKALTLRYALLPHLYTLFHQAHVAGETVARPLFLEFPKDSS
TWTVDHQLLWGEALLITPVLQAGKAEVTGYFPLGTWYDLQTVPVEALGSLPPPPAAPREPAIHSEGQWVTLPAPLDT
INVHLRAGYIIPLQGPGLTTTESRQQPMALAVALTKGGEARGELFWDDGESLEVLERGAYTQVIFLARNNTIVNELV
RVTSEGAGLQLQKVTVLGVATAPQQVLSNGVPVSNFTYSPDTKVLDICVSLLMGEQFLVSWC
(29) 69331
(SEQ ID NO: 420)
DIQLTQSPSSLSASVGDRVTITCWASQGISSYLAWYQQKPGKAPKLLIYAASTLQSGVPSRFSGSGSGTEFTLTISS
LQPEDFATYYCQQLNSYPLTFGGGTKVEIKGGGGSGGGGSGGGGSQVQLVESGGGVVQPGRSLRLSCIASGFTFSVY
GIHWVRQAPGKGLEWMAVISHDGNIKHYADSVKGRFTISRDNSKNTLYLQINSLRTEDTAVYYCAKDTWNSLDTEDI
WGQGTMVTVSSGGGGSGGGGSAHPGRPRAVPTQCDVPPNSREDCAPDKAITQEQCEARGCCYIPAKQGLQGAQMGOP
WCFFPPSYPSYKLENLSSSEMGYTATLTRTTPTFFPKDILTLRLDVMMETENRLHFTIKDPANRRYEVPLETPHVHS
RAPSPLYSVEFSEEPFGVIVRRQLDGRVLLNTTVAPLFFADQFLQLSTSLPSQYITGLAEHLSPLMLSTSWTRITLW
NRDLAPTPGANLYGSHPFYLALEDGGSAHGVFLLNSNAMDVVLQPSPALSWRSTGGILDVYIFLGPEPKSVVQQYLD
VVGYPFMPPYWGLGFHLCRWGYSSTAITRQVVENMTRAHFPLDVQWNDLDYMDSRRDFTFNKDGERDFPAMVQELHQ
GGRRYMMIVDPAISSSGPAGSYRPYDEGLRRGVFITNETGQPLIGKVWPGSTAFPDFTNPTALAWWEDMVAEFHDQV
PFDGMWIDMNEPSNFIRGSEDGCPNNELENPPYVPGVVGGTLQAATICASSHQFLSTHYNLHNLYGLTEAIASHRAL
VKARGTRPFVISRSTFAGHGRYAGHWTGDVWSSWEQLASSVPEILQFNLLGVPLVGADVCGFLGNTSEELCVRWTQL
GAFYPFMRNHNSLLSLPQEPYSFSEPAQQAMRKALTLRYALLPHLYTLFHQAHVAGETVARPLFLEFPKDSSTWTVD
HQLLWGEALLITPVLQAGKAEVTGYFPLGTWYDLQTVPVEALGSLPPPPAAPREPAIHSEGQWVTLPAPLDTINVHL
RAGYIIPLQGPGLTTTESRQQPMALAVALTKGGEARGELFWDDGESLEVLERGAYTQVIFLARNNTIVNELVRVTSE
GAGLQLQKVTVLGVATAPQQVLSNGVPVSNFTYSPDTKVLDICVSLLMGEQFLVSWC
(30) 69332
(SEQ ID NO: 421)
AIQMTQSPSSLSASVGDRVTITCRASQGIRNDLGWYQQKPGKAPKLLIYAASTLQSGVPSRFSGSGSGTDFTLTISS
LQPEDFATYYCLQDYNYPFTFGPGTKVDIKGGGGSGGGGSGGGGSQVTLRESGPALVKPSQTLTLTCTFSGFSLNTY
GMFVSWIRQPPGKALEWLAHIHWDDDKYYSTSLKTRLTISKDTSKNQVVLTMTNMDPVDTATYYCARGHNNLNYIIH
WGQGTLVTVSSGGGGSGGGGSAHPGRPRAVPTQCDVPPNSREDCAPDKAITQEQCEARGCCYIPAKQGLQGAQMGQP
WCFFPPSYPSYKLENLSSSEMGYTATLTRTTPTFFPKDILTLRLDVMMETENRLHFTIKDPANRRYEVPLETPHVHS
RAPSPLYSVEFSEEPFGVIVRRQLDGRVLLNTTVAPLFFADQFLQLSTSLPSQYITGLAEHLSPLMLSTSWTRITLW
NRDLAPTPGANLYGSHPFYLALEDGGSAHGVFLLNSNAMDVVLQPSPALSWRSTGGILDVYIFLGPEPKSVVQQYLD
VVGYPFMPPYWGLGFHLCRWGYSSTAITRQVVENMTRAHFPLDVQWNDLDYMDSRRDFTENKDGFRDFPAMVQELHQ
GGRRYMMIVDPAISSSGPAGSYRPYDEGLRRGVFITNETGQPLIGKVWPGSTAFPDFTNPTALAWWEDMVAEFHDQV
PFDGMWIDMNEPSNFIRGSEDGCPNNELENPPYVPGVVGGTLQAATICASSHQFLSTHYNLHNLYGLTEAIASHRAL
VKARGTRPFVISRSTFAGHGRYAGHWTGDVWSSWEQLASSVPEILQFNLLGVPLVGADVCGFLGNTSEELCVRWTQL
GAFYPFMRNHNSLLSLPQEPYSFSEPAQQAMRKALTLRYALLPHLYTLFHQAHVAGETVARPLFLEFPKDSSTWTVD
HQLLWGEALLITPVLQAGKAEVTGYFPLGTWYDLQTVPVEALGSLPPPPAAPREPAIHSEGQWVTLPAPLDTINVHL
RAGYIIPLQGPGLTTTESRQQPMALAVALTKGGEARGELFWDDGESLEVLERGAYTQVIFLARNNTIVNELVRVTSE
GAGLQLQKVTVLGVATAPQQVLSNGVPVSNFTYSPDTKVLDICVSLLMGEQFLVSWC
(31) 69340
(SEQ ID NO: 422)
EIVLTQSPATLSLSPGERATLSCRASQSVSSYLAWYQQKPGQAPRLLIHDVSNRATGIPARFSGSGSGTDFTLTISS
LEPEDFVVYYCQQRSDWPITFGQGTRLEIKGGGGSGGGGSGGGGSEVQLVESGGGLVQPGRSLRLSCAASGFTFDDK
AMHWVRQVPGKGLEWISGISWNSGTIGYADSVKGRFIISRDNAKNSLYLQMNSLRAEDTALYYCAKDGDTSGWYWYG
LDVWGQGTTVTVSSGGGGSGGGGSAHPGRPRAVPTQCDVPPNSREDCAPDKAITQEQCEARGCCYIPAKQGLQGAQM
GQPWCFFPPSYPSYKLENLSSSEMGYTATLTRTTPTFFPKDILTLRLDVMMETENRLHFTIKDPANRRYEVPLETPH
VHSRAPSPLYSVEFSEEPFGVIVRRQLDGRVLLNTTVAPLFFADQFLQLSTSLPSQYITGLAEHLSPLMLSTSWTRI
TLWNRDLAPTPGANLYGSHPFYLALEDGGSAHGVFLLNSNAMDVVLQPSPALSWRSTGGILDVYIFLGPEPKSVVQQ
YLDVVGYPFMPPYWGLGFHLCRWGYSSTAITRQVVENMTRAHFPLDVQWNDLDYMDSRRDFTENKDGFRDFPAMVQE
LHQGGRRYMMIVDPAISSSGPAGSYRPYDEGLRRGVFITNETGQPLIGKVWPGSTAFPDFTNPTALAWWEDMVAEFH
DQVPFDGMWIDMNEPSNFIRGSEDGCPNNELENPPYVPGVVGGTLQAATICASSHQFLSTHYNLHNLYGLTEAIASH
RALVKARGTRPFVISRSTFAGHGRYAGHWTGDVWSSWEQLASSVPEILQFNLLGVPLVGADVCGFLGNTSEELCVRW
TQLGAFYPFMRNHNSLLSLPQEPYSFSEPAQQAMRKALTLRYALLPHLYTLFHQAHVAGETVARPLFLEFPKDSSTW
TVDHQLLWGEALLITPVLQAGKAEVTGYFPLGTWYDLQTVPVEALGSLPPPPAAPREPAIHSEGQWVTLPAPLDTIN
VHLRAGYIIPLQGPGLTTTESRQQPMALAVALTKGGEARGELFWDDGESLEVLERGAYTQVIFLARNNTIVNELVRV
TSEGAGLQLQKVTVLGVATAPQQVLSNGVPVSNFTYSPDTKVLDICVSLLMGEQFLVSWC
(32) 69348
(SEQ ID NO: 423)
DIQMTQSPSSLSASVGDRVTITCRASQSIRNVLGWFQQKPGKAPQRLIYAASSLQSGVPSRFSGSGSGTEFTLTISS
LQPEDFATYYCLQHNFYPLTFGGGTKVEIKGGGGSGGGGSGGGGSQVQLVESGGGVVQPGRSLRLSCAASGFTFTTY
GMHWVRQAPGKGLEWVAVIWYDGSNKYYGDSVKGRFTISRDNSKNTLYLQMNSLRVDDTAVYYCTRTHGYTRSSDGF
DYWGQGTLVTVSSGGGGSGGGGSAHPGRPRAVPTQCDVPPNSREDCAPDKAITQEQCEARGCCYIPAKQGLQGAQMG
QPWCFFPPSYPSYKLENLSSSEMGYTATLTRTTPTFFPKDILTLRLDVMMETENRLHFTIKDPANRRYEVPLETPHV
HSRAPSPLYSVEFSEEPFGVIVRRQLDGRVLLNTTVAPLFFADQFLQLSTSLPSQYITGLAEHLSPLMLSTSWTRIT
LWNRDLAPTPGANLYGSHPFYLALEDGGSAHGVFLLNSNAMDVVLQPSPALSWRSTGGILDVYIFLGPEPKSVVQQY
LDVVGYPFMPPYWGLGFHLCRWGYSSTAITRQVVENMTRAHFPLDVQWNDLDYMDSRRDFTENKDGFRDFPAMVQEL
HQGGRRYMMIVDPAISSSGPAGSYRPYDEGLRRGVFITNETGQPLIGKVWPGSTAFPDFTNPTALAWWEDMVAEFHD
QVPFDGMWIDMNEPSNFIRGSEDGCPNNELENPPYVPGVVGGTLQAATICASSHQFLSTHYNLHNLYGLTEAIASHR
ALVKARGTRPFVISRSTFAGHGRYAGHWTGDVWSSWEQLASSVPEILQFNLLGVPLVGADVCGFLGNTSEELCVRWT
QLGAFYPFMRNHNSLLSLPQEPYSFSEPAQQAMRKALTLRYALLPHLYTLFHQAHVAGETVARPLFLEFPKDSSTWT
VDHQLLWGEALLITPVLQAGKAEVTGYFPLGTWYDLQTVPVEALGSLPPPPAAPREPAIHSEGQWVTLPAPLDTINV
HLRAGYIIPLQGPGLTTTESRQQPMALAVALTKGGEARGELFWDDGESLEVLERGAYTQVIFLARNNTIVNELVRVT
SEGAGLQLQKVTVLGVATAPQQVLSNGVPVSNFTYSPDTKVLDICVSLLMGEQFLVSWC;
however, provided herein are such fusions that are in the format V_{H}-(Gly₄Ser)₃
(SEQ ID NO: 538)-V_{L}:GAA (Gly₄Ser = SEQ ID NO: 426).

In an embodiment, an anti-hTfR scFv:GAA fusion provided herein comprises the amino acid sequence set forth in SEQ ID NO: 408 or SEQ ID NO: 405. In an embodiment, an anti-hTfR scFv:GAA fusion provided herein comprises the amino acid sequence set forth in SEQ ID NO: 408. In an embodiment, an anti-hTfR scFv:GAA fusion provided herein comprises the amino acid sequence set forth in SEQ ID NO: 405.

In an embodiment, the anti-hTfR:GAA scFv fusion protein comprises the amino acid sequence:

(1)
MHRPRRRGTRPPPLALLAALLLAARGADADIQMTQSPSSVSASVGDRVTITCRASQGIASWLAWYQQKPGKAPELLI
YAASSLQGGVPSRFSGSGSGTDFTLTISSLQPEDFAIYYCQQANYFPWTFGQGTKVEIKGGGGSGGGGSGGGGSQIT
LKESGPTLVKPTQTLTLTCTFSGFSLSTSGVGVVWIRQPPGKALEWLALIYWNDHKRYSPSLGSRLTITKDTSKNQV
VLTMTNMDPVDTATYYCAHYSGSYSYYYYGLDVWGQGTTVTVSSGGGGSGGGGSAHPGRPRAVPTQCDVPPNSRFDC
APDKAITQEQCEARGCCYIPAKQGLQGAQMGQPWCFFPPSYPSYKLENLSSSEMGYTATLTRTTPTFFPKDILTLRL
DVMMETENRLHFTIKDPANRRYEVPLETPHVHSRAPSPLYSVEFSEEPFGVIVRRQLDGRVLLNTTVAPLFFADQFL
QLSTSLPSQYITGLAEHLSPLMLSTSWTRITLWNRDLAPTPGANLYGSHPFYLALEDGGSAHGVFLLNSNAMDVVLQ
PSPALSWRSTGGILDVYIFLGPEPKSVVQQYLDVVGYPFMPPYWGLGFHLCRWGYSSTAITRQVVENMTRAHFPLDV
QWNDLDYMDSRRDFTENKDGFRDFPAMVQELHQGGRRYMMIVDPAISSSGPAGSYRPYDEGLRRGVFITNETGQPLI
GKVWPGSTAFPDFTNPTALAWWEDMVAEFHDQVPFDGMWIDMNEPSNFIRGSEDGCPNNELENPPYVPGVVGGTLQA
ATICASSHQFLSTHYNLHNLYGLTEAIASHRALVKARGTRPFVISRSTFAGHGRYAGHWTGDVWSSWEQLASSVPEI
LQFNLLGVPLVGADVCGFLGNTSEELCVRWTQLGAFYPFMRNHNSLLSLPQEPYSFSEPAQQAMRKALTLRYALLPH
LYTLFHQAHVAGETVARPLFLEFPKDSSTWTVDHQLLWGEALLITPVLQAGKAEVTGYFPLGTWYDLQTVPVEALGS
LPPPPAAPREPAIHSEGQWVTLPAPLDTINVHLRAGYIIPLQGPGLTTTESRQQPMALAVALTKGGEARGELFWDDG
ESLEVLERGAYTQVIFLARNNTIVNELVRVTSEGAGLQLQKVTVLGVATAPQQVLSNGVPVSNFTYSPDTKVLDICV
SLLMGEQFLVSWC
(SEQ ID NO: 321; optionally lacking the N-terminal
MHRPRRRGTRPPPLALLAALLLAARGADA (SEQ ID NO: 500) sequence);
(2)
MHRPRRRGTRPPPLALLAALLLAARGADADIQMTQSPSSLSASVGDRVTITCRASQSISSYLNWYQQKPGKAPKLLI
YAASSLQSGVPSRFSGSGSGTDFTLTISSLQPEDFATYYCQQSYSTPPITFGQGTRLEIKGGGGSGGGGSGGGGSQV
QLVESGGGVVQPGRSLRLSCAASGFPFSNYVMYWVRQAPGKGLEWVALIFFDGKKNYHADSVKGRFTITRDNSKNML
YLQMNSLRPEDAAVYYCAKIHCPNGVCYKGYYGMDVWGQGTTVTVSSGGGGSGGGGSAHPGRPRAVPTQCDVPPNSR
FDCAPDKAITQEQCEARGCCYIPAKQGLQGAQMGQPWCFFPPSYPSYKLENLSSSEMGYTATLTRTTPTFFPKDILT
LRLDVMMETENRLHFTIKDPANRRYEVPLETPHVHSRAPSPLYSVEFSEEPFGVIVRRQLDGRVLLNTTVAPLFFAD
QFLQLSTSLPSQYITGLAEHLSPLMLSTSWTRITLWNRDLAPTPGANLYGSHPFYLALEDGGSAHGVFLLNSNAMDV
VLQPSPALSWRSTGGILDVYIFLGPEPKSVVQQYLDVVGYPFMPPYWGLGFHLCRWGYSSTAITRQVVENMTRAHFP
LDVQWNDLDYMDSRRDFTENKDGERDFPAMVQELHQGGRRYMMIVDPAISSSGPAGSYRPYDEGLRRGVFITNETGQ
PLIGKVWPGSTAFPDFTNPTALAWWEDMVAEFHDQVPFDGMWIDMNEPSNFIRGSEDGCPNNELENPPYVPGVVGGT
LQAATICASSHQFLSTHYNLHNLYGLTEAIASHRALVKARGTRPFVISRSTFAGHGRYAGHWTGDVWSSWEQLASSV
PEILQFNLLGVPLVGADVCGFLGNTSEELCVRWTQLGAFYPFMRNHNSLLSLPQEPYSFSEPAQQAMRKALTLRYAL
LPHLYTLFHQAHVAGETVARPLFLEFPKDSSTWTVDHQLLWGEALLITPVLQAGKAEVTGYFPLGTWYDLQTVPVEA
LGSLPPPPAAPREPAIHSEGQWVTLPAPLDTINVHLRAGYIIPLQGPGLTTTESRQQPMALAVALTKGGEARGELFW
DDGESLEVLERGAYTQVIFLARNNTIVNELVRVTSEGAGLQLQKVTVLGVATAPQQVLSNGVPVSNFTYSPDTKVLD
ICVSLLMGEQFLVSWC
(SEQ ID NO: 322; optionally lacking the N-terminal
MHRPRRRGTRPPPLALLAALLLAARGADA (SEQ ID NO: 500) sequence);
(3)
MHRPRRRGTRPPPLALLAALLLAARGADADIQMTQSPSSLSASVGDRVTITCRASQSISSYLNWYQQKPGKAPKLLI
YAASSLQSGVPSRFSGSGSGTDFTLTISSLQPEDFATYYCQQSYSTPPITFGQGTRLEIKGGGGSGGGGSGGGGSEV
QLVESGGGLVQPGGSLRLSCAASGFTFNIFEMNWVRQAPGKGLEWISYISSRGTTTYYADSVRGRFTISRDNAKNSL
YLQMNSLRAEDTAVYYCARDYEATIPFDFWGQGTLVTVSSGGGGSGGGGSAHPGRPRAVPTQCDVPPNSREDCAPDK
AITQEQCEARGCCYIPAKQGLQGAQMGQPWCFFPPSYPSYKLENLSSSEMGYTATLTRTTPTFFPKDILTLRLDVMM
ETENRLHFTIKDPANRRYEVPLETPHVHSRAPSPLYSVEFSEEPFGVIVRRQLDGRVLLNTTVAPLFFADQFLQLST
SLPSQYITGLAEHLSPLMLSTSWTRITLWNRDLAPTPGANLYGSHPFYLALEDGGSAHGVFLLNSNAMDVVLQPSPA
LSWRSTGGILDVYIFLGPEPKSVVQQYLDVVGYPFMPPYWGLGFHLCRWGYSSTAITRQVVENMTRAHFPLDVQWND
LDYMDSRRDFTFNKDGFRDFPAMVQELHQGGRRYMMIVDPAISSSGPAGSYRPYDEGLRRGVFITNETGQPLIGKVW
PGSTAFPDFTNPTALAWWEDMVAEFHDQVPFDGMWIDMNEPSNFIRGSEDGCPNNELENPPYVPGVVGGTLQAATIC
ASSHQFLSTHYNLHNLYGLTEAIASHRALVKARGTRPFVISRSTFAGHGRYAGHWTGDVWSSWEQLASSVPEILQEN
LLGVPLVGADVCGFLGNTSEELCVRWTQLGAFYPFMRNHNSLLSLPQEPYSFSEPAQQAMRKALTLRYALLPHLYTL
FHQAHVAGETVARPLFLEFPKDSSTWTVDHQLLWGEALLITPVLQAGKAEVTGYFPLGTWYDLQTVPVEALGSLPPP
PAAPREPAIHSEGQWVTLPAPLDTINVHLRAGYIIPLQGPGLTTTESRQQPMALAVALTKGGEARGELFWDDGESLE
VLERGAYTQVIFLARNNTIVNELVRVTSEGAGLQLQKVTVLGVATAPQQVLSNGVPVSNFTYSPDTKVLDICVSLLM
GEQFLVSWC
(SEQ ID NO: 323; optionally lacking the N-terminal
MHRPRRRGTRPPPLALLAALLLAARGADA (SEQ ID NO: 500) sequence);
or
(4)
MHRPRRRGTRPPPLALLAALLLAARGADADIQMTQSPSSLSASVGDRVTITCRASQSISSYLNWYQQKPGKAPKLLI
YAASSLQSGVPSRFSGSGSGTDFTLTISSLQPEDFATYYCQQSYSTPPITFGQGTRLEIKGGGGSGGGGSGGGGSEV
QLVESGGGLVQPGRSLRLSCAASGFTFDDYAMNWVRQAPGKGLEWVSGISWSSGSMDYADSVKGRFTISRDNAKNSL
YLQMNSLRTEDTALYYCAKAREVGDYYGMDVWGQGTTVTVSSGGGGSGGGGSAHPGRPRAVPTQCDVPPNSREDCAP
DKAITQEQCEARGCCYIPAKQGLQGAQMGQPWCFFPPSYPSYKLENLSSSEMGYTATLTRTTPTFFPKDILTLRLDV
MMETENRLHFTIKDPANRRYEVPLETPHVHSRAPSPLYSVEFSEEPFGVIVRRQLDGRVLLNTTVAPLFFADQFLQL
STSLPSQYITGLAEHLSPLMLSTSWTRITLWNRDLAPTPGANLYGSHPFYLALEDGGSAHGVFLLNSNAMDVVLQPS
PALSWRSTGGILDVYIFLGPEPKSVVQQYLDVVGYPFMPPYWGLGFHLCRWGYSSTAITRQVVENMTRAHFPLDVQW
NDLDYMDSRRDFTFNKDGFRDFPAMVQELHQGGRRYMMIVDPAISSSGPAGSYRPYDEGLRRGVFITNETGQPLIGK
VWPGSTAFPDFTNPTALAWWEDMVAEFHDQVPFDGMWIDMNEPSNFIRGSEDGCPNNELENPPYVPGVVGGTLQAAT
ICASSHQFLSTHYNLHNLYGLTEAIASHRALVKARGTRPFVISRSTFAGHGRYAGHWTGDVWSSWEQLASSVPEILQ
FNLLGVPLVGADVCGFLGNTSEELCVRWTQLGAFYPFMRNHNSLLSLPQEPYSFSEPAQQAMRKALTLRYALLPHLY
TLFHQAHVAGETVARPLFLEFPKDSSTWTVDHQLLWGEALLITPVLQAGKAEVTGYFPLGTWYDLQTVPVEALGSLP
PPPAAPREPAIHSEGQWVTLPAPLDTINVHLRAGYIIPLQGPGLTTTESRQQPMALAVALTKGGEARGELFWDDGES
LEVLERGAYTQVIFLARNNTIVNELVRVTSEGAGLQLQKVTVLGVATAPQQVLSNGVPVSNFTYSPDTKVLDICVSL
LMGEQFLVSWC
(SEQ ID NO: 324; optionally lacking the N-terminal
MHRPRRRGTRPPPLALLAALLLAARGADA (SEQ ID NO: 500) sequence).

Fab fragments that bind specifically to human transferrin receptor, optionally fused to a payload such as GAA (or variant thereof) (anti-TfR Fab:Payload fusion proteins), are provided herein. Fab fragments typically contain one complete light chain, V_{L}, and a constant light domain, e.g., kappa (e.g., RTVAAPSVFIFPPSDEQLKSGTASVVCLLNNFYPREAKVQWKVDNALQSGNSQESVTEQDSKDSTYSLS STLTLSKADYEKHKVYACEVTHQGLSSPVTKSFNRGEC (SEQ ID NO: 424)) and the V_{H} and IgG1 CH1 portion (e.g., ASTKGPSVFPLAPSSKSTSGGTAALGCLVKDYFPEPVTVSWNSGALTSGVHTFPAVLQSSGLYSLSSVV TVPSSSLGTQTYICNVNHKPSNTKVDKKVEPKSCDKTH (SEQ ID NO: 425)) or IgG4 CH1 (e.g., ASTKGPSVFPLAPCSRSTSESTAALGCLVKDYFPEPVTVSWNSGALTSGVHTFPAVLQSSGLYSLSSVVTVPSSSLG TKTYTCNVDHKPSNTKVDKRVESKYGPPLLQGSG (SEQ ID NO: 459); or ASTKGPSVFPLAPCSRSTSESTAALGCLVKDYFPEPVTVSWNSGALTSGVHTFPAVLQSSGLYSLSSVVTVPSSSLG TKTYTCNVDHKPSNTKVDKRVESKYGPP (SEQ ID NO: 493)) of one heavy chain. Fab fragment antibodies can be generated by papain digestion of whole IgG antibodies to remove the entire Fc fragment, including the hinge region. For example, provided herein are Fab proteins comprising:
(1) a heavy chain variable region (HCVR) that comprises the amino acid sequence set forth in SEQ ID NO: 2 or 462, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 7, or LCDR1, LCDR2 and LCDR3 of such a LCVR-linked to the CL domain;
(2) a heavy chain variable region (HCVR) that comprises the amino acid sequence set forth in SEQ ID NO: 12 or 463, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 17, or LCDR1, LCDR2 and LCDR3 of such a LCVR-linked to the CL domain;
(3) a heavy chain variable region (HCVR) that comprises the amino acid sequence set forth in SEQ ID NO: 22 or 464, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 27, or LCDR1, LCDR2 and LCDR3 of such a LCVR-linked to the CL domain;
(4) a heavy chain variable region (HCVR) that comprises the amino acid sequence set forth in SEQ ID NO: 32, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 37 or 465, or LCDR1, LCDR2 and LCDR3 of such a LCVR-linked to the CL domain;
(5) a heavy chain variable region (HCVR) that comprises the amino acid sequence set forth in SEQ ID NO: 42, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 47 or 466, or LCDR1, LCDR2 and LCDR3 of such a LCVR-linked to the CL domain;
(6) a heavy chain variable region (HCVR) that comprises the amino acid sequence set forth in SEQ ID NO: 52 or 467, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 57 or 468, or LCDR1, LCDR2 and LCDR3 of such a LCVR-linked to the CL domain;
(7) a heavy chain variable region (HCVR) that comprises the amino acid sequence set forth in SEQ ID NO: 62 or 492, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 67 or 469, or LCDR1, LCDR2 and LCDR3 of such a LCVR-linked to the CL domain;
(8) a heavy chain variable region (HCVR) that comprises the amino acid sequence set forth in SEQ ID NO: 72 or 470, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 77 or 471, or LCDR1, LCDR2 and LCDR3 of such a LCVR-linked to the CL domain;
(9) a heavy chain variable region (HCVR) that comprises the amino acid sequence set forth in SEQ ID NO: 82, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 87, or LCDR1, LCDR2 and LCDR3 of such a LCVR-linked to the CL domain;
(10) a heavy chain variable region (HCVR) that comprises the amino acid sequence set forth in SEQ ID NO: 92 or 472, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 97, or LCDR1, LCDR2 and LCDR3 of such a LCVR-linked to the CL domain;
(11) a heavy chain variable region (HCVR) that comprises the amino acid sequence set forth in SEQ ID NO: 102, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 107, or LCDR1, LCDR2 and LCDR3 of such a LCVR-linked to the CL domain;
(12) a heavy chain variable region (HCVR) that comprises the amino acid sequence set forth in SEQ ID NO: 112 or 473, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 117 or 474, or LCDR1, LCDR2 and LCDR3 of such a LCVR-linked to the CL domain;
(13) a heavy chain variable region (HCVR) that comprises the amino acid sequence set forth in SEQ ID NO: 122, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 127, or LCDR1, LCDR2 and LCDR3 of such a LCVR-linked to the CL domain;
(14) a heavy chain variable region (HCVR) that comprises the amino acid sequence set forth in SEQ ID NO: 132, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 137, or LCDR1, LCDR2 and LCDR3 of such a LCVR-linked to the CL domain;
(15) a heavy chain variable region (HCVR) that comprises the amino acid sequence set forth in SEQ ID NO: 142 or 475, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 147 or 476, or LCDR1, LCDR2 and LCDR3 of such a LCVR-linked to the CL domain;
(16) a heavy chain variable region (HCVR) that comprises the amino acid sequence set forth in SEQ ID NO: 152, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 157, or LCDR1, LCDR2 and LCDR3 of such a LCVR-linked to the CL domain;
(17) a heavy chain variable region (HCVR) that comprises the amino acid sequence set forth in SEQ ID NO: 162 or 477, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 167, or LCDR1, LCDR2 and LCDR3 of such a LCVR-linked to the CL domain;
(18) a heavy chain variable region (HCVR) that comprises the amino acid sequence set forth in SEQ ID NO: 172, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 177, or LCDR1, LCDR2 and LCDR3 of such a LCVR-linked to the CL domain;
(19) a heavy chain variable region (HCVR) that comprises the amino acid sequence set forth in SEQ ID NO: 182 or 478, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 187 or 479, or LCDR1, LCDR2 and LCDR3 of such a LCVR-linked to the CL domain;
(20) a heavy chain variable region (HCVR) that comprises the amino acid sequence set forth in SEQ ID NO: 192 or 480, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 197, or LCDR1, LCDR2 and LCDR3 of such a LCVR-linked to the CL domain;
(21) a heavy chain variable region (HCVR) that comprises the amino acid sequence set forth in SEQ ID NO: 202 or 481, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 207, or LCDR1, LCDR2 and LCDR3 of such a LCVR-linked to the CL domain;
(22) a heavy chain variable region (HCVR) that comprises the amino acid sequence set forth in SEQ ID NO: 212, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 217, or LCDR1, LCDR2 and LCDR3 of such a LCVR-linked to the CL domain;
(23) a heavy chain variable region (HCVR) that comprises the amino acid sequence set forth in SEQ ID NO: 222, or a heavy chain variable region that includes HCDR1, HCDR2 and HCDR3 of such a HCVR-linked to the CH1 domain-and comprising a light chain variable region (LCVR) that comprises the amino acid sequence set forth in SEQ ID NO: 227, or LCDR1, LCDR2 and LCDR3 of such a LCVR-linked to the CL domain;
(24) a heavy chain variable region (HCVR) that comprises the amino acid sequence set forth in SEQ ID NO: 232, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 237, or LCDR1, LCDR2 and LCDR3 of such a LCVR-linked to the CL domain;
(25) a heavy chain variable region (HCVR) that comprises the amino acid sequence set forth in SEQ ID NO: 242, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 247, or LCDR1, LCDR2 and LCDR3 of such a LCVR-linked to the CL domain;
(26) a heavy chain variable region (HCVR) that comprises the amino acid sequence set forth in SEQ ID NO: 252 or 482, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 257, or LCDR1, LCDR2 and LCDR3 of such a LCVR-linked to the CL domain;
(27) a heavy chain variable region (HCVR) that comprises the amino acid sequence set forth in SEQ ID NO: 262, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 267, or LCDR1, LCDR2 and LCDR3 of such a LCVR-linked to the CL domain;
(28) a heavy chain variable region (HCVR) that comprises the amino acid sequence set forth in SEQ ID NO: 272, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 277, or LCDR1, LCDR2 and LCDR3 of such a LCVR-linked to the CL domain;
(29) a heavy chain variable region (HCVR) that comprises the amino acid sequence set forth in SEQ ID NO: 282, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 287, or LCDR1, LCDR2 and LCDR3 of such a LCVR-linked to the CL domain;
(30) a heavy chain variable region (HCVR) that comprises the amino acid sequence set forth in SEQ ID NO: 292, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 297, or LCDR1, LCDR2 and LCDR3 of such a LCVR-linked to the CL domain;
(31) a heavy chain variable region (HCVR) that comprises the amino acid sequence set forth in SEQ ID NO: 302 or 483, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 307 or 488, or LCDR1, LCDR2 and LCDR3 of such a LCVR-linked to the CL domain; and/or
(32) a heavy chain variable region (HCVR) that comprises the amino acid sequence set forth in SEQ ID NO: 312, or a heavy chain variable region that includes HCDR1, HCDR2 and HCDR3 of such a HCVR-linked to the CH1 domain-and
a light chain variable region (LCVR) that comprises the amino acid sequence set forth in SEQ ID NO: 317 or 484, or LCDR1, LCDR2 and LCDR3 of such a LCVR-linked to the CL domain;
e.g., wherein CH1 is from IgG1 or IgG4;
e.g., wherein CH1 is SEQ ID NO: 425, 459, or 493.

For example, provided herein are Fab proteins comprising: (23) a heavy chain variable region (HCVR) that comprises the amino acid sequence set forth in SEQ ID NO: 222, or a heavy chain variable region that includes HCDR1, HCDR2 and HCDR3 of such a HCVR-linked to the CH1 domain-and comprising a light chain variable region (LCVR) that comprises the amino acid sequence set forth in SEQ ID NO: 227, or LCDR1, LCDR2 and LCDR3 of such a LCVR-linked to the CL domain; or (25) a heavy chain variable region (HCVR) that comprises the amino acid sequence set forth in SEQ ID NO: 242, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 247, or LCDR1, LCDR2 and LCDR3 of such a LCVR-linked to the CL domain. For example, provided herein are Fab proteins comprising: (23) a heavy chain variable region (HCVR) that comprises the amino acid sequence set forth in SEQ ID NO: 222, or a heavy chain variable region that includes HCDR1, HCDR2 and HCDR3 of such a HCVR-linked to the CH1 domain-and comprising a light chain variable region (LCVR) that comprises the amino acid sequence set forth in SEQ ID NO: 227, or LCDR1, LCDR2 and LCDR3 of such a LCVR-linked to the CL domain. For example, provided herein are Fab proteins comprising: (25) a heavy chain variable region (HCVR) that comprises the amino acid sequence set forth in SEQ ID NO: 242, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 247, or LCDR1, LCDR2 and LCDR3 of such a LCVR-linked to the CL domain. For example, provided herein are Fab proteins comprising: (14) a heavy chain variable region (HCVR) that comprises the amino acid sequence set forth in SEQ ID NO: 132, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 137, or LCDR1, LCDR2 and LCDR3 of such a LCVR-linked to the CL domain. For example, provided herein are Fab proteins comprising: (18) a heavy chain variable region (HCVR) that comprises the amino acid sequence set forth in SEQ ID NO: 172, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 177, or LCDR1, LCDR2 and LCDR3 of such a LCVR-linked to the CL domain. For example, provided herein are Fab proteins comprising: (27) a heavy chain variable region (HCVR) that comprises the amino acid sequence set forth in SEQ ID NO: 262, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 267, or LCDR1, LCDR2 and LCDR3 of such a LCVR-linked to the CL domain. For example, provided herein are Fab proteins comprising: (28) a heavy chain variable region (HCVR) that comprises the amino acid sequence set forth in SEQ ID NO: 272, or a heavy chain variable region that includes HCDR1, HCDR2 and HCDR3 of such a HCVR-linked to the CH1 domain-and a light chain variable region (LCVR) that comprises the amino acid sequence set forth in SEQ ID NO: 277, or LCDR1, LCDR2 and LCDR3 of such a LCVR-linked to the CL domain.

In an embodiment, the antigen-binding fragment comprises a Fab protein. In an embodiment, the Fab protein comprises the amino acid sequences set forth in SEQ ID NO: 372 and SEQ ID NO: 496, 487, or 373 (or variants thereof) or comprises the amino acid sequences set forth in SEQ ID NO: 376 and SEQ ID NO: 497, 489, or 377 (or variants thereof). In an embodiment, the Fab protein comprises the amino acid sequences set forth in SEQ ID NO: 372 and SEQ ID NO: 496, 487, or 373 (or variants thereof). In an embodiment, the Fab protein comprises the amino acid sequences set forth in SEQ ID NO: 376 and SEQ ID NO: 497, 489, or 377 (or variants thereof).

Heavy and light chains of anti-hTfR Fabs in exemplary anti-hTfR:Payload fusion proteins provided herein are set forth below.

(1) 31874B
Fab Light Chain
(SEQ ID NO: 328)
DIQMTQSPSSLSASVGDRVTITCRASQGISNYLAWYQQKPGKVPNLLIYAASTLQSGVPSRFSGSGSGTDFTLTISS
LQPEDVATYYCQKYNSAPLTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSENRGEC
Fab Heavy Chain
(SEQ ID NO: 329)
EVQLVESGGGLVQPGGSLRLSCAASGFAFSSYAMTWVRQAPGKGLEWVSVISGTGGSTYYADSVKGRFTISRDNSKN
TLYLQMNSLRAEDTAVYYCAKGGAARRMEYFQYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTH
(2) 31863B
Fab Light Chain
(SEQ ID NO: 330)
DIQMTQSPSSLSASIGDRVTITCRASQGISNYLAWYQQKPGKVPKLLIYAASTLQSGVPSRFSGSGSGTDFTLTISS
LQPEDVATYYCQNHNSVPLTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSENRGEC
Fab Heavy Chain
(SEQ ID NO: 331)
EVQLVESGGGLVQPGGSLRLSCAASGFTFNSYAMTWVRQAPGKGLEWVSFIGGSTGNTYYAGSVKGRFTISSDNSKK
TLYLQMNSLRAEDTAVYYCAKGGAARRMEYFQHWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTH
(3) 69348
Fab Light Chain
(SEQ ID NO: 332)
DIQMTQSPSSLSASVGDRVTITCRASQSIRNVLGWFQQKPGKAPQRLIYAASSLQSGVPSRFSGSGSGTEFTLTISS
LQPEDFATYYCLQHNFYPLTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSENRGEC
Fab Heavy Chain
(SEQ ID NO: 333)
QVQLVESGGGVVQPGRSLRLSCAASGFTFTTYGMHWVRQAPGKGLEWVAVIWYDGSNKYYGDSVKGRFTISRDNSKN
TLYLQMNSLRVDDTAVYYCTRTHGYTRSSDGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
(4) 69340
Fab Light Chain
(SEQ ID NO: 334)
EIVLTQSPATLSLSPGERATLSCRASQSVSSYLAWYQQKPGQAPRLLIHDVSNRATGIPARFSGSGSGTDFTLTISS
LEPEDFVVYYCQQRSDWPITFGQGTRLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 335)
EVQLVESGGGLVQPGRSLRLSCAASGFTFDDKAMHWVRQVPGKGLEWISGISWNSGTIGYADSVKGRFIISRDNAKN
SLYLQMNSLRAEDTALYYCAKDGDTSGWYWYGLDVWGQGTTVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKTH
(5) 69331
Fab Light Chain
(SEQ ID NO: 336)
DIQLTQSPSSLSASVGDRVTITCWASQGISSYLAWYQQKPGKAPKLLIYAASTLQSGVPSRFSGSGSGTEFTLTISS
LQPEDFATYYCQQLNSYPLTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSENRGEC
Fab Heavy Chain
(SEQ ID NO: 337)
QVQLVESGGGVVQPGRSLRLSCIASGFTFSVYGIHWVRQAPGKGLEWMAVISHDGNIKHYADSVKGRFTISRDNSKN
TLYLQINSLRTEDTAVYYCAKDTWNSLDTFDIWGQGTMVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTH
(6) 69332
Fab Light Chain
(SEQ ID NO: 338)
AIQMTQSPSSLSASVGDRVTITCRASQGIRNDLGWYQQKPGKAPKLLIYAASTLQSGVPSRFSGSGSGTDFTLTISS
LQPEDFATYYCLQDYNYPFTFGPGTKVDIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSENRGEC
Fab Heavy Chain
(SEQ ID NO: 339)
QVTLRESGPALVKPSQTLTLTCTFSGFSLNTYGMFVSWIRQPPGKALEWLAHIHWDDDKYYSTSLKTRLTISKDTSK
NQVVLTMTNMDPVDTATYYCARGHNNLNYIIHWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTH
(7) 69326
Fab Light Chain
(SEQ ID NO: 340)
EIVMTQSPATLSVSPGERATLSCRASQSVSSNFAWYQQKPGQAPRLLIYSASSRATGIPVRFSGSGSGTEFTLTISS
LQSEDFAVYYCQQYNIWPRTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSENRGEC
Fab Heavy Chain
(SEQ ID NO: 341)
EVQLVESGGGLVQPGGSLRLSCAVSGFIFSSYEMNWVRQAPGKGLEWVSYISSSGSTIFYADSVKGRFTISRDNAKN
SLYLQMNSLRAEDTAVYYCVSGVVLFDVWGQGTMVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTH
(8) 69329
Fab Light Chain
(SEQ ID NO: 342)
DIQMTQSPSSVSASVGDRVTITCRASQGISSWLAWYQQKPGKAPKLLIYAASSLQSGVPSRFSGSGSGTDFTLTISS
LQPEDFATYYCQKANSFPYTFGQGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 343)
EVQLVESGGGLVQPGGSLRLSCAASGFTFSNYWMTWVRQAPGKGLEWVANIKEDGSEKDYVDSVKGRFTISRDNAKN
SLYLQMNSLRGEDTAVYYCARDGEQLVDYYYYYVMDVWGQGTTVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCDKT
H
(9) 69323
Fab Light Chain
(SEQ ID NO: 344)
DIQMTQSPSSLSASVGDRVTITCRASQSISSYLNWYQQKPGKAPKVLIYAASSLQSGVPSRESGSGSGTDFTLTISS
LQPEDFATYYCQQSYSIPLTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSENRGEC
Fab Heavy Chain
(SEQ ID NO: 345)
EVQLVESGGGLVQPGRSLRLSCAASGFTEDDYAMHWVRQAPGKGLEWVSGISWNSGYIGYADSVKGRFTISRDNAEN
SLHLQMNSLRAEDTALYYCARGGSTLVRGVKGGYYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKKVEPKSCD
KTH
(10) 69305
Fab Light Chain
(SEQ ID NO: 346)
DIQMTQSPSSLSASVGDRVTITCRASQSIDRYLNWYRQKPGKAPKLLIYTTSSLQSGVPSRFSGSGSGTDFTLTLSS
LQPEDFATYYCQQSYSPPLTFGGGTKVEIKRTVAAPSVFIFPPSDEQLKSGTASVVCLLNNFYPREAKVOWKVDNAL
QSGNSQESVTEQDSKDSTYSLSSTLTLSKADYEKHKVYACEVTHQGLSSPVTKSFNRGEC
Fab Heavy Chain
(SEQ ID NO: 347)
QVQLVESGGGVVQPGRSLRLSCAASGFTFSSYGMHWVRQAPGKGLEWVAVIWYDGSNKYYADSVKGRFTISRDISKN
TLYLQMNSLRAEDTAVYYCAGQLDLFFDYWGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKVDKKVEPKSCDKTH
(11) 69307
Fab Light Chain
(SEQ ID NO: 348)
DIQMTQSPSSVSASVGDRVTITCRASQGISSWLAWYQQKPGKAPKLLIYAASSLQSGVPSRFSGSGSGTDFTLTISS
LQPEDFATYYCQKADSLPYAFGQGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSENRGEC
Fab Heavy Chain
(SEQ ID NO: 349)
EVQLVESGGGLVQPGGSLRLSCTASGFTFSNYWMTWVRQAPGKGLEWVANIKEDGSEKEYVDSVKGRFTISRDNAKN
SLYLQMNSLRGEDTAVYYCARDGEQLVDYYYYYVMDVWGQGTTVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCDKT
H
(12) 12795B
Fab Light Chain
(SEQ ID NO: 350)
DIQMTQSPSSLSASVGDRVTITCRASQGIRDHFGWYQQKPGKAPKRLIYAASSLHSGVPSRFSGSGSGTEFTLTISS
LQPEDFATYYCLQYDTYPLTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSENRGEC
Fab Heavy Chain
(SEQ ID NO: 351)
EVQLVESGGGLVQPGGSLRLSCATSGFTFTSYDMKWVRQAPGLGLEWVSAISGSGGNTYYADSVKGRFTISRDNSRN
TLYLQMNSLRAEDTAVYYCTRSHDFGAFDYFDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTH
(13) 12798B (REGN17078)
Fab Light Chain
(SEQ ID NO: 352)
EIVMTQSPATLSVSPGERATLSCRASQTVSSNLAWYQQKPGQAPRLLIYGSSSRATGIPARFSGSGSGTEFTLTISS
LQSEDFAVYYCQQYNNWPPYTFGQGTKLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSENRGEC
Fab Heavy Chain
(SEQ ID NO: 353)
EVQLVESGGDLVQPGRSLRLSCAASGFTFDDYAMHWVRQAPGKGLEWVSGISWNSATRVYADSVKGRFTISRDNAKN
FLYLQMNSLRSEDTALYHCAKDMDISLGYYGLDVWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH;
or
(SEQ ID NO: 485)
EVQLVESGGDLVQPGRSLRLSCAASGFTFDDYAMHWVRQAPGKGLEWVSGISWNSATRVYADSVKGRFTISRDNAKN
FLYLQMNSLRSEDTALYHCAKDMDISLGYYGLDVWGQGTTVTVSSASTKGPSVFPLAPCSRSTSESTAALGCLVKDY
FPEPVTVSWNSGALTSGVHTFPAVLQSSGLYSLSSVVTVPSSSLGTKTYTCNVDHKPSNTKVDKRVESKYGPPLLQG
SG;
or
(SEQ ID NO: 494)
EVQLVESGGDLVQPGRSLRLSCAASGFTEDDYAMHWVRQAPGKGLEWVSGISWNSATRVYADSVKGRFTISRDNAKN
FLYLQMNSLRSEDTALYHCAKDMDISLGYYGLDVWGQGTTVTVSSASTKGPSVFPLAPCSRSTSESTAALGCLVKDY
FPEPVTVSWNSGALTSGVHTFPAVLQSSGLYSLSSVVTVPSSSLGTKTYTCNVDHKPSNTKVDKRVESKYGPP
(14) 12799B (REGN17079)
Fab Light Chain
(SEQ ID NO: 354)
DIQMTQSPSSVSASVGDRVTITCRASQGIASWLAWYQQKPGKAPELLIYAASSLQGGVPSRFSGSGSGTDFTLTISS
LQPEDFAIYYCQQANYFPWTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSENRGEC
Fab Heavy Chain
(SEQ ID NO: 355)
QITLKESGPTLVKPTQTLTLTCTFSGFSLSTSGVGVVWIRQPPGKALEWLALIYWNDHKRYSPSLGSRLTITKDTSK
NQVVLTMTNMDPVDTATYYCAHYSGSYSYYYYGLDVWGQGTTVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSCDKTH;
(SEQ ID NO: 486)
QITLKESGPTLVKPTQTLTLTCTFSGFSLSTSGVGVVWIRQPPGKALEWLALIYWNDHKRYSPSLGSRLTITKDTSK
NQVVLTMTNMDPVDTATYYCAHYSGSYSYYYYGLDVWGQGTTVTVSSASTKGPSVFPLAPCSRSTSESTAALGCLVK
DYFPEPVTVSWNSGALTSGVHTFPAVLQSSGLYSLSSVVTVPSSSLGTKTYTCNVDHKPSNTKVDKRVESKYGPPLL
QGSG;
or
(SEQ ID NO: 495)
QITLKESGPTLVKPTQTLTLTCTFSGFSLSTSGVGVVWIRQPPGKALEWLALIYWNDHKRYSPSLGSRLTITKDTSK
NQVVLTMTNMDPVDTATYYCAHYSGSYSYYYYGLDVWGQGTTVTVSSASTKGPSVFPLAPCSRSTSESTAALGCLVK
DYFPEPVTVSWNSGALTSGVHTFPAVLQSSGLYSLSSVVTVPSSSLGTKTYTCNVDHKPSNTKVDKRVESKYGPP
(15) 12801B
Fab Light Chain
(SEQ ID NO: 356)
DIQMTQSPSSLSASVGDRVTITCRASQGIRTDLGWYQQKPGKAPKRLIYAASSLQSGVPSRFSGSGSGTEFTLTISS
LRPEDFATFYCLQYNSYPLTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSENRGEC
Fab Heavy Chain
(SEQ ID NO: 357)
EVQLLESGGALVQPGGSLRLSCAASGFTFTSYAMHWVRQAPGKGLEWVSSIRGSGGGTYSADSVKGRFTISRDNSRD
TLYLQMNSVRAEDTAVYYCARSHDYGAFDFFDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTH
(16) 12802B
Fab Light Chain
(SEQ ID NO: 358)
EIVMTQSPATLSVSPGERATLSCRASQSVSINLAWYQQKPGQAPRLLIFVASTRATGIPARFSGSGSGTEFTLTISS
LQSEDFATYYCQQYDIWPYTFGQGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNRGEC
Fab Heavy Chain
(SEQ ID NO: 359)
QVQLVESGGGLVKPGGSLRLSCAASGFTFSDYFMSWIRQAPGKGLEWVSYISSTGSTINYADSVKGRFTISRDNVKN
SLYLQMTSLRVEDTAVYYCTRDNWNYEYWGQGTLVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVDKKVEPKSCDKTH
(17) 12808B
Fab Light Chain
(SEQ ID NO: 360)
DIQMTQSPSSLSASVGDRVTINCRASQGIRNDLGWYQQKPGKAPKRLIYAASSLQSGVPLRFSGSGSGTEFTLTINN
LQPEDFATYYCLSHNSYPWTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSENRGEC
Fab Heavy Chain
(SEQ ID NO: 361)
QLQLQESGPGLVKPSETLSLTCTVSGESISSNTYYWGWIRQPPGKGLEWIGSIDYSGTTNYNPSLKSRVTISVDTSR
NHFSLRLRSVTAADTAVYYCAREWGNYGYYYGMDVWGQGTTVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKTH
(18) 12812B
Fab Light Chain
(SEQ ID NO: 362)
DIQMTQSPPSVSASVGDRVTITCRASQGISSWLAWYQQKPGKAPKLLIYAASSLQSGVPSRFSGSGSGTDFTLTISS
LQPEDFATYYCQQANSFPRTFGQGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSENRGEC
Fab Heavy Chain
(SEQ ID NO: 363)
QVQLVQSGAEVKKPGSSVRVSCKASRGTFSSYAISWVRQAPGQGLEWMGGIIPIFGTANYAQKFLARVTITADESTS
TAYMELSSLRSEDTAVYYCAREKGWNYFDYWGQGTLVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVDKKVEPKSCDKTH
(19) 12816B
Fab Light Chain
(SEQ ID NO: 364)
DIVMTQSPLSLPVTPGEPASISCRSSQSLLHGNGYNYLTWYLQKPGQSPQLLIYLGSNRASGVPDRESGSGSGTDFT
LKISRVEAEDVGVYYCMQALQTPYTFGQGTKLEIKRTVAAPSVFIFPPSDEQLKSGTASVVCLLNNFYPREAKVOWK
VDNALQSGNSQESVTEQDSKDSTYSLSSTLTLSKADYEKHKVYACEVTHQGLSSPVTKSENRGEC
Fab Heavy Chain
(SEQ ID NO: 365)
QVQLVESGGGLVKPGGSLRLSCAASGFTFSDYYMNWIRQAPGKGLEWVSYISSSGTTIYYADSVKGRFTISRDNAKK
SLYLEMNSLRAEDTAVYYCAREGYGNDYYYYGIDVWGQGTTVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKTH
(20) 12833B
Fab Light Chain
(SEQ ID NO: 366)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSENRGEC
Fab Heavy Chain
(SEQ ID NO: 367)
QVQLVESGGGVVQPGRSLRLSCAASGFTFSSFGMHWVRQAPGKGLEWVIFISYDGSDKYYADSVKGRFAISRDSSKN
TLYLQMNSLRAEDTAVYYCAKENGILTDSYGMDVWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
(21) 12834B
Fab Light Chain
(SEQ ID NO: 368)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSENRGEC
Fab Heavy Chain
(SEQ ID NO: 369)
QVQLVQSGAEVKKPGASVKVSCKASGYTFTSYGISWVRQAPGQGLEWMGWISVYHGNTNYAQKFQGRVTMTTDTSTS
TAYMELRSLRSDDTAVYYCAREGYYDFWSGYYPFDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQTYICNVNHKPSNTKVDKKVEPKSCDKTH
(22) 12835B
Fab Light Chain
(SEQ ID NO: 370)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSENRGEC
Fab Heavy Chain
(SEQ ID NO: 371)
EVQLVESGGGLIQPGGSLRLSCEASGFTFRNYEMNWVRQAPGKGLEWVSYISSSGNMKDYAESVKGRFTISRDNVKN
SLQLQMNSLRVEDTAVYYCARDEFPYGMDVWGQGTTVTVSSASTKGPSVFPLAPSSKSTSGGTAALGCLVKDYFPEP
VTVSWNSGALTSGVHTFPAVLQSSGLYSLSSVVTVPSSSLGTQTYICNVNHKPSNTKVDKKVEPKSCDKTH
(23) 12847B (REGN17083)
Fab Light Chain
(SEQ ID NO: 372)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSENRGEC
Fab Heavy Chain
(SEQ ID NO: 373)
EVQLVESGGGLVQPGRSLRLSCAASGFTEDDYAMNWVRQAPGKGLEWVSGISWSSGSMDYADSVKGRFTISRDNAKN
SLYLQMNSLRTEDTALYYCAKAREVGDYYGMDVWGQGTTVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTH;
(SEQ ID NO: 487)
EVQLVESGGGLVQPGRSLRLSCAASGFTEDDYAMNWVRQAPGKGLEWVSGISWSSGSMDYADSVKGRFTISRDNAKN
SLYLQMNSLRTEDTALYYCAKAREVGDYYGMDVWGQGTTVTVSSASTKGPSVFPLAPCSRSTSESTAALGCLVKDYF
PEPVTVSWNSGALTSGVHTFPAVLQSSGLYSLSSVVTVPSSSLGTKTYTCNVDHKPSNTKVDKRVESKYGPPLLQGS
G;
or
(SEQ ID NO: 496)
EVQLVESGGGLVQPGRSLRLSCAASGFTFDDYAMNWVRQAPGKGLEWVSGISWSSGSMDYADSVKGRFTISRDNAKN
SLYLQMNSLRTEDTALYYCAKAREVGDYYGMDVWGQGTTVTVSSASTKGPSVFPLAPCSRSTSESTAALGCLVKDYF
PEPVTVSWNSGALTSGVHTFPAVLQSSGLYSLSSVVTVPSSSLGTKTYTCNVDHKPSNTKVDKRVESKYGPP
(24) 12848B
Fab Light Chain
(SEQ ID NO: 374)
EIVLTQSPGTLSLSPGERATLSCRASQSVSSSYLAWYQQKPGQAPRLLIYGASSRATGIPDRFSGSGSGTDFTLTIS
RLEPEDFAVYYCQQYGSSPWTFGQGTKVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSENRGEC
Fab Heavy Chain
(SEQ ID NO: 375)
EVQLVESGGGLVQPGRSLTLSCAASGFTFDNFGMHWVRQGPGKGLEWVSGLTWNSGVIGYADSVKGRFTISRDNAKN
SLYLQMNSLRPEDTALYYCAKDIRNYGPFDYWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVDKKVEPKSCDKTH
(25) 12843B (REGN17081)
Fab Light Chain
(SEQ ID NO: 376)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSENRGEC
Fab Heavy Chain
(SEQ ID NO: 377)
EVQLVESGGGLVQPGGSLRLSCAASGFTFNIFEMNWVRQAPGKGLEWISYISSRGTTTYYADSVRGRFTISRDNAKN
SLYLQMNSLRAEDTAVYYCARDYEATIPFDFWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVDKKVEPKSCDKTH;
(SEQ ID NO: 489)
EVQLVESGGGLVQPGGSLRLSCAASGFTFNIFEMNWVRQAPGKGLEWISYISSRGTTTYYADSVRGRFTISRDNAKN
SLYLQMNSLRAEDTAVYYCARDYEATIPFDFWGQGTLVTVSSASTKGPSVFPLAPCSRSTSESTAALGCLVKDYFPE
PVTVSWNSGALTSGVHTFPAVLQSSGLYSLSSVVTVPSSSLGTKTYTCNVDHKPSNTKVDKRVESKYGPPLLQGSG;
or
(SEQ ID NO: 497)
EVQLVESGGGLVQPGGSLRLSCAASGFTFNIFEMNWVRQAPGKGLEWISYISSRGTTTYYADSVRGRFTISRDNAKN
SLYLQMNSLRAEDTAVYYCARDYEATIPFDFWGQGTLVTVSSASTKGPSVFPLAPCSRSTSESTAALGCLVKDYFPE
PVTVSWNSGALTSGVHTFPAVLQSSGLYSLSSVVTVPSSSLGTKTYTCNVDHKPSNTKVDKRVESKYGPP
(26) 12844B
Fab Light Chain
(SEQ ID NO: 378)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC
Fab Heavy Chain
(SEQ ID NO: 379)
EVQLVESGGSVVRPGGSLRLSCEASGFTFDDYGMSWVRQDPGKGLEWVSGINWNGDRTNYADSVKGRFIISRDNAKN
SVYLQMNSLRAEDSALYHCARDQGLGVAATLDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYICNVNHKPSNTKVDKKVEPKSCDKTH
(27) 12845B (REGN17082)
Fab Light Chain
(SEQ ID NO: 380)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSENRGEC
Fab Heavy Chain
(SEQ ID NO: 381)
EVQLVESGGGLVQPGGSLRLSCAASGFTVSNYEMNWVRQAPGKGLEWVSYISSSTSNIYYADSVKGRFTISRDNAEN
SLYLQMNSLRVEDTAVYYCVRDGIVVVPVGRGYYYYGLDVWGQGTTVTVSSASTKGPSVFPLAPSSKSTSGGTAALG
CLVKDYFPEPVTVSWNSGALTSGVHTFPAVLQSSGLYSLSSVVTVPSSSLGTQTYICNVNHKPSNTKVDKKVEPKSC
DKTH;
(SEQ ID NO: 490)
EVQLVESGGGLVQPGGSLRLSCAASGFTVSNYEMNWVRQAPGKGLEWVSYISSSTSNIYYADSVKGRFTISRDNAEN
SLYLQMNSLRVEDTAVYYCVRDGIVVVPVGRGYYYYGLDVWGQGTTVTVSSASTKGPSVFPLAPCSRSTSESTAALG
CLVKDYFPEPVTVSWNSGALTSGVHTFPAVLQSSGLYSLSSVVTVPSSSLGTKTYTCNVDHKPSNTKVDKRVESKYG
PPLLQGSG;
or
(SEQ ID NO: 498)
EVQLVESGGGLVQPGGSLRLSCAASGFTVSNYEMNWVRQAPGKGLEWVSYISSSTSNIYYADSVKGRFTISRDNAEN
SLYLQMNSLRVEDTAVYYCVRDGIVVVPVGRGYYYYGLDVWGQGTTVTVSSASTKGPSVFPLAPCSRSTSESTAALG
CLVKDYFPEPVTVSWNSGALTSGVHTFPAVLQSSGLYSLSSVVTVPSSSLGTKTYTCNVDHKPSNTKVDKRVESKYG
PP
(28) 12839B (REGN17080)
Fab Light Chain
(SEQ ID NO: 382)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSENRGEC
Fab Heavy Chain
(SEQ ID NO: 383)
QVQLVESGGGVVQPGRSLRLSCAASGFPFSNYVMYWVRQAPGKGLEWVALIFFDGKKNYHADSVKGRFTITRDNSKN
MLYLQMNSLRPEDAAVYYCAKIHCPNGVCYKGYYGMDVWGQGTTVTVSSASTKGPSVFPLAPSSKSTSGGTAALGCL
VKDYFPEPVTVSWNSGALTSGVHTFPAVLQSSGLYSLSSVVTVPSSSLGTQTYICNVNHKPSNTKVDKKVEPKSCDK
TH;
(SEQ ID NO: 491)
QVQLVESGGGVVQPGRSLRLSCAASGFPFSNYVMYWVRQAPGKGLEWVALIFFDGKKNYHADSVKGRFTITRDNSKN
MLYLQMNSLRPEDAAVYYCAKIHCPNGVCYKGYYGMDVWGQGTTVTVSSASTKGPSVFPLAPCSRSTSESTAALGCL
VKDYFPEPVTVSWNSGALTSGVHTFPAVLQSSGLYSLSSVVTVPSSSLGTKTYTCNVDHKPSNTKVDKRVESKYGPP
LLQGSG;
or
(SEQ ID NO: 499)
QVQLVESGGGVVQPGRSLRLSCAASGFPFSNYVMYWVRQAPGKGLEWVALIFFDGKKNYHADSVKGRFTITRDNSKN
MLYLQMNSLRPEDAAVYYCAKIHCPNGVCYKGYYGMDVWGQGTTVTVSSASTKGPSVFPLAPCSRSTSESTAALGCL
VKDYFPEPVTVSWNSGALTSGVHTFPAVLQSSGLYSLSSVVTVPSSSLGTKTYTCNVDHKPSNTKVDKRVESKYGPP
(29) 12841B
Fab Light Chain
(SEQ ID NO: 384)
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSENRGEC
Fab Heavy Chain
(SEQ ID NO: 385)
EVQLVESGGGLVQPGGSLRLSCAASGFTFSNYWMNWVRQAPGKGLEWVANIKEDGGKKLYVDSVKGRFTISRDNAKN
SLFLQMNSLRAEDTAVYYCAREDTTLVVDYYYYGMDVWGQGTTVTVSSASTKGPSVFPLAPSSKSTSGGTAALGCLV
KDYFPEPVTVSWNSGALTSGVHTFPAVLQSSGLYSLSSVVTVPSSSLGTQTYICNVNHKPSNTKVDKKVEPKSCDKT
H
(30) 12850B
Fab Light Chain
(SEQ ID NO: 386)
EIVLTQSPGTLSLSPGERATLSCRASQSVSSSYLAWYQQKPGQAPRLLIYGASSRATGIPDRESGSGSGTDETLTIS
RLEPEDFAVYYCQQYGSSPWTFGQGTKVEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSENRGEC
Fab Heavy Chain
(SEQ ID NO: 387)
QVQLVQSGAEVKKPGSSVKVSCKASGGTFNTYAITWVRQAPGQGLEWMGGIIPISGIAEYAQKFQGRVTITTDDSST
TAYMELNSLRSEDTAVYYCASWNYALYYFYGMDVWGRGTTVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH
(31) 69261
Fab Light Chain
(SEQ ID NO: 388)
DIVMTQSPLSLPVTPGEPASISCRSSQSLLHSNGYNYLDWYLQKPGQSPQFLIYLGSNRASGVPDRESGSGSGTDFT
LKINRVEAEDVGVYYCMQALQTPYTFGQGTKLEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSENRGEC
Fab Heavy Chain
(SEQ ID NO: 389)
QVQLVESGGGLVKPGGSLRLSCAASGFTFSVYYMNWIRQAPGKGLEWVSYISSSGSTIYYADSVKGRFTISRDNAKN
SLYLQMNSLRAEDTAVYYCGREGYSGTYSYYGMDVWGQGTTVTVSSASTKGPSVFPLAPSSKSTSGGTAALGCLVKD
YFPEPVTVSWNSGALTSGVHTFPAVLQSSGLYSLSSVVTVPSSSLGTQTYICNVNHKPSNTKVDKKVEPKSCDKTH
(32) 69263
Fab Light Chain
(SEQ ID NO: 390)
DIQMTQSPSSLSASVGDRVTITCRASQDISHYSAWYQQKPGKLPNLLIYAASTLQSGVPSRFSGSGSGTDESLTTSS
LQPEDVATYYCQKYNSVPLTFGGGTKVEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSENRGEC
Fab Heavy Chain
(SEQ ID NO: 391)
EVQLVESGGGLVQPGRSLRLSCAVSGFTFDDYAMHWVRQAPGKGLEWVSGISWNSGTRGYADSVKGRFTISRDNAKN
SLYLQMNSLRGEDTALYYCVKDITISPNYYGMDVWGQGTTVTVSSASTKGPSVFPLAPSSKSTSGGTAALGCLVKDY
FPEPVTVSWNSGALTSGVHTFPAVLQSSGLYSLSSVVTVPSSSLGTQTYICNVNHKPSNTKVDKKVEPKSCDKTH

In an embodiment, an anti-TfR antigen-binding protein, e.g., antibody or antigen-binding fragment (which may be tethered to a payload) comprises an IgG1 heavy chain constant domain comprising the sequence set forth in SEQ ID NO: 571: ASTKGPSVFPLAPSSKSTSGGTAALGCLVKDYFPEPVTVSWNSGALTSGVHTFPAVLQSSGLYSLSSVVTVPSSSLG TQTYICNVNHKPSNTKVDKKVEPKSCDKTHTCPPCPAPELLGGPSVFLFPPKPKDTLMISRTPEVTCVVVDVSHEDP EVKFNWYVDGVEVHNAKTKPREEQYNSTYRVVSVLTVLHQDWLNGKEYKCKVSNKALPAPIEKTISKAKGQPREPQV YTLPPSRDELTKNQVSLTCLVKGFYPSDIAVEWESNGQPENNYKTTPPVLDSDGSFFLYSKLTVDKSRWQQGNVFSC SVMHEALHNHYTQKSLSLSPGK (see, e.g., sequences of Table B, or variants thereof). In an embodiment, an antigen-binding protein, e.g., antibody or antigen-binding fragment, comprises a light chain constant domain, e.g., of the type kappa or lambda. In an embodiment, a V_{H} as set forth herein is linked to a human heavy chain constant domain (e.g., IgG) and a V_{L} as set forth herein is linked to a human light chain constant domain (e.g., kappa). The present disclosure includes antigen-binding proteins comprising the variable domains set forth herein, which are linked to a heavy and/or light chain constant domain, e.g., as set forth herein.

TABLE B
Heavy Chain Full hlgG1 Sequences.

| Identifier | HC Full hIgG1 sequence |
|---|---|
| 31874B | EVQLVESGGGLVQPGGSLRLSCAASGFAFSSYAMTWVRQAPGKGLEWVSVISGTGGSTYYADSVKG |
| | RFTISRDNSKNTLYLQMNSLRAEDTAVYYCAKGGAARRMEYFQYWGQGTLVTVSSASTKGPSVFPL |
| | APSSKSTSGGTAALGCLVKDYFPEPVTVSWNSGALTSGVHTFPAVLQSSGLYSLSSVVTVPSSSLG |
| | TQTYICNVNHKPSNTKVDKKVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK (SEQ ID NO: |
| | 539) |
| 31863B | EVQLVESGGGLVQPGGSLRLSCAASGFTENSYAMTWVRQAPGKGLEWVSFIGGSTGNTYYAGSVKG |
| | RFTISSDNSKKTLYLQMNSLRAEDTAVYYCAKGGAARRMEYFQHWGQGTLVTVSSASTKGPSVFPL |
| | APSSKSTSGGTAALGCLVKDYFPEPVTVSWNSGALTSGVHTFPAVLQSSGLYSLSSVVTVPSSSLG |
| | TQTYICNVNHKPSNTKVDKKVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK (SEQ ID NO: |
| | 540) |
| 69348 | QVQLVESGGGVVQPGRSLRLSCAASGFTFTTYGMHWVRQAPGKGLEWVAVIWYDGSNKYYGDSVKG |
| | RFTISRDNSKNTLYLQMNSLRVDDTAVYYCTRTHGYTRSSDGFDYWGQGTLVTVSSASTKGPSVFP |
| | LAPSSKSTSGGTAALGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK (SEQ ID NO: |
| | 541) |
| 69340 | EVQLVESGGGLVQPGRSLRLSCAASGFTFDDKAMHWVRQVPGKGLEWISGISWNSGTIGYADSVKG |
| | RFIISRDNAKNSLYLQMNSLRAEDTALYYCAKDGDTSGWYWYGLDVWGQGTTVTVSSASTKGPSVF |
| | PLAPSSKSTSGGTAALGCLVKDYFPEPVTVSWNSGALTSGVHTFPAVLQSSGLYSLSSVVTVPSSS |
| | LGTQTYICNVNHKPSNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSPGK (SEQ ID NO: |
| | 542) |
| 69331 | QVQLVESGGGVVQPGRSLRLSCIASGFTFSVYGIHWVRQAPGKGLEWMAVISHDGNIKHYADSVKG |
| | RFTISRDNSKNTLYLQINSLRTEDTAVYYCAKDTWNSLDTFDIWGQGTMVTVSSASTKGPSVFPLA |
| | PSSKSTSGGTAALGCLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKPKDTLMISRTPEVTC |
| | VVVDVSHEDPEVKFNWYVDGVEVHNAKTKPREEQYNSTYRVVSVLTVLHQDWLNGKEYKCKVSNKA |
| | LPAPIEKTISKAKGQPREPQVYTLPPSRDELTKNQVSLTCLVKGFYPSDIAVEWESNGQPENNYKT |
| | TPPVLDSDGSFFLYSKLTVDKSRWQQGNVFSCSVMHEALHNHYTQKSLSLSPGK (SEQ ID NO: |
| | 543) |
| 69332 | QVTLRESGPALVKPSQTLTLTCTFSGFSLNTYGMFVSWIRQPPGKALEWLAHIHWDDDKYYSTSLK |
| | TRLTISKDTSKNQVVLTMTNMDPVDTATYYCARGHNNLNYIIHWGQGTLVTVSSASTKGPSVFPLA |
| | PSSKSTSGGTAALGCLVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPKPKDTLMISRTPEVTC |
| | VVVDVSHEDPEVKFNWYVDGVEVHNAKTKPREEQYNSTYRVVSVLTVLHQDWLNGKEYKCKVSNKA |
| | LPAPIEKTISKAKGQPREPQVYTLPPSRDELTKNQVSLTCLVKGFYPSDIAVEWESNGQPENNYKT |
| | TPPVLDSDGSFFLYSKLTVDKSRWQQGNVFSCSVMHEALHNHYTQKSLSLSPGK (SEQ ID NO: |
| | 544) |
| 69326 | EVQLVESGGGLVQPGGSLRLSCAVSGFIFSSYEMNWVRQAPGKGLEWVSYISSSGSTIFYADSVKG |
| | RFTISRDNAKNSLYLQMNSLRAEDTAVYYCVSGVVLFDVWGQGTMVTVSSASTKGPSVFPLAPSSK |
| | STSGGTAALGCLVKDYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQDWLNGKEYKCKVSNKALPAP |
| | IEKTISKAKGQPREPQVYTLPPSRDELTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK (SEQ ID NO: 545) |
| 69329 | EVQLVESGGGLVQPGGSLRLSCAASGFTFSNYWMTWVRQAPGKGLEWVANIKEDGSEKDYVDSVKG |
| | RFTISRDNAKNSLYLQMNSLRGEDTAVYYCARDGEQLVDYYYYYVMDVWGQGTTVTVSSASTKGPS |
| | VFPLAPSSKSTSGGTAALGCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSLSPGK (SEQ ID |
| | NO: 546) |
| 69323 | EVQLVESGGGLVQPGRSLRLSCAASGFTFDDYAMHWVRQAPGKGLEWVSGISWNSGYIGYADSVKG |
| | RFTISRDNAENSLHLQMNSLRAEDTALYYCARGGSTLVRGVKGGYYGMDVWGQGTTVTVSSASTKG |
| | PSVFPLAPSSKSTSGGTAALGCLVKDYFPEPVTVSWNSGALTSGVHTFPAVLQSSGLYSLSSVVTV |
| | PSSSLGTQTYICNVNHKPSNTKVDKKVEPKSCDKTHTCPPCPAPELLGGPSVFLFPPKPKDTLMIS |
| | RTPEVTCVVVDVSHEDPEVKFNWYVDGVEVHNAKTKPREEQYNSTYRVVSVLTVLHQDWLNGKEYK |
| | CKVSNKALPAPIEKTISKAKGQPREPQVYTLPPSRDELTKNQVSLTCLVKGFYPSDIAVEWESNGQ |
| | PENNYKTTPPVLDSDGSFFLYSKLTVDKSRWQQGNVFSCSVMHEALHNHYTQKSLSLSPGK (SEQ |
| | ID NO: 547) |
| 69305 | QVQLVESGGGVVQPGRSLRLSCAASGFTFSSYGMHWVRQAPGKGLEWVAVIWYDGSNKYYADSVKG |
| | RFTISRDISKNTLYLQMNSLRAEDTAVYYCAGQLDLFFDYWGQGTLVTVSSASTKGPSVFPLAPSS |
| | KSTSGGTAALGCLVKDYFPEPVTVSWNSGALTSGVHTFPAVLQSSGLYSLSSVVTVPSSSLGTQTY |
| | ICNVNHKPSNTKVDKKVEPKSCDKTHTCPPCPAPELLGGPSVFLFPPKPKDTLMISRTPEVTCVVV |
| | DVSHEDPEVKFNWYVDGVEVHNAKTKPREEQYNSTYRVVSVLTVLHQDWLNGKEYKCKVSNKALPA |
| | PIEKTISKAKGQPREPQVYTLPPSRDELTKNQVSLTCLVKGFYPSDIAVEWESNGQPENNYKTTPP |
| | VLDSDGSFFLYSKLTVDKSRWQQGNVFSCSVMHEALHNHYTQKSLSLSPGK (SEQ ID NO: 548) |
| 69307 | EVQLVESGGGLVQPGGSLRLSCTASGFTFSNYWMTWVRQAPGKGLEWVANIKEDGSEKEYVDSVKG |
| | RFTISRDNAKNSLYLQMNSLRGEDTAVYYCARDGEQLVDYYYYYVMDVWGQGTTVTVSSASTKGPS |
| | VFPLAPSSKSTSGGTAALGCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSLSPGK (SEQ ID |
| | NO: 549) |
| 12795B | EVQLVESGGGLVQPGGSLRLSCATSGFTFTSYDMKWVRQAPGLGLEWVSAISGSGGNTYYADSVKG |
| | RFTISRDNSRNTLYLQMNSLRAEDTAVYYCTRSHDFGAFDYFDYWGQGTLVTVSSASTKGPSVEPL |
| | APSSKSTSGGTAALGCLVKDYFPEPVTVSWNSGALTSGVHTFPAVLQSSGLYSLSSVVTVPSSSLG |
| | TQTYICNVNHKPSNTKVDKKVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK (SEQ ID NO: |
| | 550) |
| 12798B | EVQLVESGGDLVQPGRSLRLSCAASGFTFDDYAMHWVRQAPGKGLEWVSGISWNSATRVYADSVKG |
| | RFTISRDNAKNFLYLQMNSLRSEDTALYHCAKDMDISLGYYGLDVWGQGTTVTVSSASTKGPSVEP |
| | LAPSSKSTSGGTAALGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK (SEQ ID NO: |
| | 551) |
| 12799B | QITLKESGPTLVKPTQTLTLTCTFSGFSLSTSGVGVVWIRQPPGKALEWLALIYWNDHKRYSPSLG |
| | SRLTITKDTSKNQVVLTMTNMDPVDTATYYCAHYSGSYSYYYYGLDVWGQGTTVTVSSASTKGPSV |
| | FPLAPSSKSTSGGTAALGCLVKDYFPEPVTVSWNSGALTSGVHTFPAVLQSSGLYSLSSVVTVPSS |
| | SLGTQTYICNVNHKPSNTKVDKKVEPKSCDKTHTCPPCPAPELLGGPSVFLFPPKPKDTLMISRTP |
| | EVTCVVVDVSHEDPEVKFNWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPSDIAVEWESNGOPEN |
| | NYKTTPPVLDSDGSFFLYSKLTVDKSRWQQGNVFSCSVMHEALHNHYTQKSLSLSPGK (SEQ ID |
| | NO: 552) |
| 12801B | EVQLLESGGALVQPGGSLRLSCAASGFTFTSYAMHWVRQAPGKGLEWVSSIRGSGGGTYSADSVKG |
| | RFTISRDNSRDTLYLQMNSVRAEDTAVYYCARSHDYGAFDFFDYWGQGTLVTVSSASTKGPSVFPL |
| | APSSKSTSGGTAALGCLVKDYFPEPVTVSWNSGALTSGVHTFPAVLQSSGLYSLSSVVTVPSSSLG |
| | TQTYICNVNHKPSNTKVDKKVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK (SEQ ID NO: |
| | 553) |
| 12802B | QVQLVESGGGLVKPGGSLRLSCAASGFTFSDYFMSWIRQAPGKGLEWVSYISSTGSTINYADSVKG |
| | RFTISRDNVKNSLYLQMTSLRVEDTAVYYCTRDNWNYEYWGQGTLVTVSSASTKGPSVFPLAPSSK |
| | STSGGTAALGCLVKDYFPEPVTVSWNSGALTSGVHTFPAVLQSSGLYSLSSVVTVPSSSLGTQTYI |
| | CNVNHKPSNTKVDKKVEPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQDWLNGKEYKCKVSNKALPAP |
| | IEKTISKAKGQPREPQVYTLPPSRDELTKNQVSLTCLVKGFYPSDIAVEWESNGQPENNYKTTPPV |
| | LDSDGSFFLYSKLTVDKSRWQQGNVFSCSVMHEALHNHYTQKSLSLSPGK (SEQ ID NO: 554) |
| 12808B | QLQLQESGPGLVKPSETLSLTCTVSGESISSNTYYWGWIRQPPGKGLEWIGSIDYSGTTNYNPSLK |
| | SRVTISVDTSRNHFSLRLRSVTAADTAVYYCAREWGNYGYYYGMDVWGQGTTVTVSSASTKGPSVF |
| | PLAPSSKSTSGGTAALGCLVKDYFPEPVTVSWNSGALTSGVHTFPAVLQSSGLYSLSSVVTVPSSS |
| | LGTQTYICNVNHKPSNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSPGK (SEQ ID NO: |
| | 555) |
| 12812B | QVQLVQSGAEVKKPGSSVRVSCKASRGTESSYAISWVRQAPGQGLEWMGGIIPIFGTANYAQKFLA |
| | RVTITADESTSTAYMELSSLRSEDTAVYYCAREKGWNYFDYWGQGTLVTVSSASTKGPSVFPLAPS |
| | SKSTSGGTAALGCLVKDYFPEPVTVSWNSGALTSGVHTFPAVLQSSGLYSLSSVVTVPSSSLGTQT |
| | YICNVNHKPSNTKVDKKVEPKSCDKTHTCPPCPAPELLGGPSVFLFPPKPKDTLMISRTPEVTCVV |
| | VDVSHEDPEVKFNWYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQPENNYKTTP |
| | PVLDSDGSFFLYSKLTVDKSRWQQGNVFSCSVMHEALHNHYTQKSLSLSPGK (SEQ ID NO: 556) |
| 12816B | QVQLVESGGGLVKPGGSLRLSCAASGFTFSDYYMNWIRQAPGKGLEWVSYISSSGTTIYYADSVKG |
| | RFTISRDNAKKSLYLEMNSLRAEDTAVYYCAREGYGNDYYYYGIDVWGQGTTVTVSSASTKGPSVF |
| | PLAPSSKSTSGGTAALGCLVKDYFPEPVTVSWNSGALTSGVHTFPAVLQSSGLYSLSSVVTVPSSS |
| | LGTQTYICNVNHKPSNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSPGK (SEQ ID NO: |
| | 557) |
| 12833B | QVQLVESGGGVVQPGRSLRLSCAASGFTFSSFGMHWVRQAPGKGLEWVIFISYDGSDKYYADSVKG |
| | RFAISRDSSKNTLYLQMNSLRAEDTAVYYCAKENGILTDSYGMDVWGQGTTVTVSSASTKGPSVFP |
| | LAPSSKSTSGGTAALGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK (SEQ ID NO: |
| | 558) |
| 12834B | QVQLVQSGAEVKKPGASVKVSCKASGYTFTSYGISWVRQAPGQGLEWMGWISVYHGNTNYAQKFQG |
| | RVTMTTDTSTSTAYMELRSLRSDDTAVYYCAREGYYDFWSGYYPFDYWGQGTLVTVSSASTKGPSV |
| | FPLAPSSKSTSGGTAALGCLVKDYFPEPVTVSWNSGALTSGVHTFPAVLQSSGLYSLSSVVTVPSS |
| | SLGTQTYICNVNHKPSNTKVDKKVEPKSCDKTHTCPPCPAPELLGGPSVFLFPPKPKDTLMISRTP |
| | EVTCVVVDVSHEDPEVKFNWYVDGVEVHNAKTKPREEQYNSTYRVVSVLTVLHQDWLNGKEYKCKV |
| | SNKALPAPIEKTISKAKGQPREPQVYTLPPSRDELTKNQVSLTCLVKGFYPSDIAVEWESNGOPEN |
| | NYKTTPPVLDSDGSFFLYSKLTVDKSRWQQGNVFSCSVMHEALHNHYTQKSLSLSPGK (SEQ ID |
| | NO: 559) |
| 12835B | EVQLVESGGGLIQPGGSLRLSCEASGFTFRNYEMNWVRQAPGKGLEWVSYISSSGNMKDYAESVKG |
| | RFTISRDNVKNSLQLQMNSLRVEDTAVYYCARDEFPYGMDVWGQGTTVTVSSASTKGPSVFPLAPS |
| | SKSTSGGTAALGCLVKDYFPEPVTVSWNSGALTSGVHTFPAVLQSSGLYSLSSVVTVPSSSLGTQT |
| | YICNVNHKPSNTKVDKKVEPKSCDKTHTCPPCPAPELLGGPSVFLFPPKPKDTLMISRTPEVTCVV |
| | VDVSHEDPEVKFNWYVDGVEVHNAKTKPREEQYNSTYRVVSVLTVLHQDWLNGKEYKCKVSNKALP |
| | APIEKTISKAKGQPREPQVYTLPPSRDELTKNQVSLTCLVKGFYPSDIAVEWESNGQPENNYKTTP |
| | PVLDSDGSFFLYSKLTVDKSRWQQGNVFSCSVMHEALHNHYTQKSLSLSPGK (SEQ ID NO: 560) |
| 12847B | EVQLVESGGGLVQPGRSLRLSCAASGFTEDDYAMNWVRQAPGKGLEWVSGISWSSGSMDYADSVKG |
| | RFTISRDNAKNSLYLQMNSLRTEDTALYYCAKAREVGDYYGMDVWGQGTTVTVSSASTKGPSVFPL |
| | APSSKSTSGGTAALGCLVKDYFPEPVTVSWNSGALTSGVHTFPAVLQSSGLYSLSSVVTVPSSSLG |
| | TQTYICNVNHKPSNTKVDKKVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK (SEQ ID NO: |
| | 561) |
| 12848B | EVQLVESGGGLVQPGRSLTLSCAASGFTFDNFGMHWVRQGPGKGLEWVSGLTWNSGVIGYADSVKG |
| | RFTISRDNAKNSLYLQMNSLRPEDTALYYCAKDIRNYGPFDYWGQGTLVTVSSASTKGPSVFPLAP |
| | SSKSTSGGTAALGCLVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVSNKAL |
| | PAPIEKTISKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGK (SEQ ID NO: 562) |
| 12843B | EVQLVESGGGLVQPGGSLRLSCAASGFTFNIFEMNWVRQAPGKGLEWISYISSRGTTTYYADSVRG |
| | RFTISRDNAKNSLYLQMNSLRAEDTAVYYCARDYEATIPFDFWGQGTLVTVSSASTKGPSVFPLAP |
| | SSKSTSGGTAALGCLVKDYFPEPVTVSWNSGALTSGVHTFPAVLQSSGLYSLSSVVTVPSSSLGTQ |
| | TYICNVNHKPSNTKVDKKVEPKSCDKTHTCPPCPAPELLGGPSVFLFPPKPKDTLMISRTPEVTCV |
| | VVDVSHEDPEVKFNWYVDGVEVHNAKTKPREEQYNSTYRVVSVLTVLHQDWLNGKEYKCKVSNKAL |
| | PAPIEKTISKAKGQPREPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSLSPGK (SEQ ID NO: 563) |
| 12844B | EVQLVESGGSVVRPGGSLRLSCEASGFTFDDYGMSWVRQDPGKGLEWVSGINWNGDRTNYADSVKG |
| | RFIISRDNAKNSVYLQMNSLRAEDSALYHCARDQGLGVAATLDYWGQGTLVTVSSASTKGPSVFPL |
| | APSSKSTSGGTAALGCLVKDYFPEPVTVSWNSGALTSGVHTFPAVLQSSGLYSLSSVVTVPSSSLG |
| | TQTYICNVNHKPSNTKVDKKVEPKSCDKTHTCPPCPAPELLGGPSVFLFPPKPKDTLMISRTPEVT |
| | CVVVDVSHEDPEVKFNWYVDGVEVHNAKTKPREEQYNSTYRVVSVLTVLHQDWLNGKEYKCKVSNK |
| | ALPAPIEKTISKAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPGK (SEQ ID NO: |
| | 564) |
| 12845B | EVQLVESGGGLVQPGGSLRLSCAASGFTVSNYEMNWVRQAPGKGLEWVSYISSSTSNIYYADSVKG |
| | RFTISRDNAENSLYLQMNSLRVEDTAVYYCVRDGIVVVPVGRGYYYYGLDVWGQGTTVTVSSASTK |
| | GPSVFPLAPSSKSTSGGTAALGCLVKDYFPEPVTVSWNSGALTSGVHTFPAVLQSSGLYSLSSVVT |
| | VPSSSLGTQTYICNVNHKPSNTKVDKKVEPKSCDKTHTCPPCPAPELLGGPSVFLFPPKPKDTLMI |
| | SRTPEVTCVVVDVSHEDPEVKFNWYVDGVEVHNAKTKPREEQYNSTYRVVSVLTVLHQDWLNGKEY |
| | KCKVSNKALPAPIEKTISKAKGQPREPQVYTLPPSRDELTKNQVSLTCLVKGFYPSDIAVEWESNG |
| | QPENNYKTTPPVLDSDGSFFLYSKLTVDKSRWQQGNVFSCSVMHEALHNHYTQKSLSLSPGK (SEQ |
| | ID NO: 565) |
| 12839B | QVQLVESGGGVVQPGRSLRLSCAASGFPFSNYVMYWVRQAPGKGLEWVALIFFDGKKNYHADSVKG |
| | RFTITRDNSKNMLYLQMNSLRPEDAAVYYCAKIHCPNGVCYKGYYGMDVWGQGTTVTVSSASTKGP |
| | SVFPLAPSSKSTSGGTAALGCLVKDYFPEPVTVSWNSGALTSGVHTFPAVLQSSGLYSLSSVVTVP |
| | SSSLGTQTYICNVNHKPSNTKVDKKVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVSVLTVLHQDWLNGKEYKC |
| | KVSNKALPAPIEKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLSLSPGK (SEQ ID |
| | NO: 566) |
| 12841B | EVQLVESGGGLVQPGGSLRLSCAASGFTFSNYWMNWVRQAPGKGLEWVANIKEDGGKKLYVDSVKG |
| | RFTISRDNAKNSLFLQMNSLRAEDTAVYYCAREDTTLVVDYYYYGMDVWGQGTTVTVSSASTKGPS |
| | VFPLAPSSKSTSGGTAALGCLVKDYFPEPVTVSWNSGALTSGVHTFPAVLQSSGLYSLSSVVTVPS |
| | SSLGTQTYICNVNHKPSNTKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVSVLTVLHQDWLNGKEYKCK |
| | VSNKALPAPIEKTISKAKGQPREPQVYTLPPSRDELTKNQVSLTCLVKGFYPSDIAVEWESNGQPE |
| | NNYKTTPPVLDSDGSFFLYSKLTVDKSRWQQGNVFSCSVMHEALHNHYTQKSLSLSPGK (SEQ ID |
| | NO: 567) |
| 12850B | QVQLVQSGAEVKKPGSSVKVSCKASGGTENTYAITWVRQAPGQGLEWMGGIIPISGIAEYAQKFQG |
| | RVTITTDDSSTTAYMELNSLRSEDTAVYYCASWNYALYYFYGMDVWGRGTTVTVSSASTKGPSVFP |
| | LAPSSKSTSGGTAALGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK (SEQ ID NO: |
| | 568) |
| 69261 | QVQLVESGGGLVKPGGSLRLSCAASGFTFSVYYMNWIRQAPGKGLEWVSYISSSGSTIYYADSVKG |
| | RFTISRDNAKNSLYLQMNSLRAEDTAVYYCGREGYSGTYSYYGMDVWGQGTTVTVSSASTKGPSVF |
| | PLAPSSKSTSGGTAALGCLVKDYFPEPVTVSWNSGALTSGVHTFPAVLQSSGLYSLSSVVTVPSSS |
| | LGTQTYICNVNHKPSNTKVDKKVEPKSCDKTHTCPPCPAPELLGGPSVFLFPPKPKDTLMISRTPE |
| | VTCVVVDVSHEDPEVKFNWYVDGVEVHNAKTKPREEQYNSTYRVVSVLTVLHQDWLNGKEYKCKVS |
| | NKALPAPIEKTISKAKGQPREPQVYTLPPSRDELTKNQVSLTCLVKGFYPSDIAVEWESNGQPENN |
| | YKTTPPVLDSDGSFFLYSKLTVDKSRWQQGNVFSCSVMHEALHNHYTQKSLSLSPGK (SEQ ID NO: |
| | 569) |
| 69263 | EVQLVESGGGLVQPGRSLRLSCAVSGFTEDDYAMHWVRQAPGKGLEWVSGISWNSGTRGYADSVKG |
| | RFTISRDNAKNSLYLQMNSLRGEDTALYYCVKDITISPNYYGMDVWGQGTTVTVSSASTKGPSVFP |
| | LAPSSKSTSGGTAALGCLVKDYFPEPVTVSWNSGALTSGVHTFPAVLQSSGLYSLSSVVTVPSSSL |
| | GTQTYICNVNHKPSNTKVDKKVEPKSCDKTHTCPPCPAPELLGGPSVFLFPPKPKDTLMISRTPEV |
| | TCVVVDVSHEDPEVKFNWYVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFYPSDIAVEWESNGQPENNY |
| | KTTPPVLDSDGSFFLYSKLTVDKSRWQQGNVFSCSVMHEALHNHYTQKSLSLSPGK (SEQ ID NO: |
| | 570) |

“31874B”; “31863B”; “69348”; “69340”; “69331”; “69332”; “69326”; “69329”; “69323”; “69305”; “69307”; “12795B”; “12798B”; “12799B”; “12801B”; “12802B”; “12808B”; “12812B”; “12816B”; “12833B”; “12834B”; “12835B”; “12847B”; “12848B”; “12843B”; “12844B”; “12845B”; “12839B”; “12841B”; “12850B”; “69261”; and “69263” refer to anti-TfR:Payload fusion proteins, e.g., anti-TfR scFv:GAA or anti-TfR Fab:GAA, comprising a light chain variable region comprising the amino acid sequence set forth in SEQ ID NO: 7; 17; 27; 37; 465; 47; 466; 57; 468; 67; 469; 77; 471; 87; 97; 107; 117; 474; 127; 137; 147; 476; 157; 167; 177; 187; 479; 197; 207; 217; 227; 237; 247; 257; 267; 277; 287; 297; 307; 488; 317 or 484 (or a variant thereof), and a heavy chain variable region comprising the amino acid sequence set forth in SEQ ID NO: 2; 462; 12; 463; 22; 464; 32; 42; 52; 467; 62; 492; 72; 470; 82; 92; 472; 102; 112; 473; 122; 132; 142; 475; 152; 162; 477; 172; 182; 478; 192; 480; 202; 481; 212; 222; 232; 242; 252; 482; 262; 272; 282; 292; 302; 483 or 312 (or a variant thereof); which, in the case of an scFv, can be fused together (in either order), e.g., by a peptide linker (e.g., (G₄S)₃ (SEQ ID NO: 538)), respectively; or that comprise a V_{H} that comprises the CDRs thereof (CDR-H1 (or a variant thereof), CDR-H2 (or a variant thereof) and CDR-H3 (or a variant thereof)) and/or a V_{L} that comprises the CDRs thereof (CDR-L1 (or a variant thereof), CDR-L2 (or a variant thereof) and CDR-L3 (or a variant thereof)), wherein the V_{H} fused to the V_{L} or the V_{L} fused to the V_{H}, in the case of an scFv, can be fused, e.g., by a peptide linker (e.g., (G₄S)₂ (SEQ ID NO: 537)), to a payload such as GAA polypeptide or variant thereof.

In some embodiments, the anti-TfR antigen-binding protein described herein comprises a humanized antibody or antigen binding fragment thereof, murine antibody or antigen binding fragment thereof, chimeric antibody or antigen binding fragment thereof, monoclonal antibody or antigen binding fragment thereof (e.g., monovalent Fab′, divalent Fab2, F(ab)′3 fragments, single-chain variable fragment (scFv), bis-scFv, (scFv)2, diabody, bivalent antibody, one-armed antibody, minibody, nanobody, triabody, tetrabody, disulfide stabilized Fv protein (dsFv), single-domain antibody (sdAb), Ig NAR, camelid antibody or antigen binding fragment thereof, bispecific antibody or biding fragment thereof, (e.g., bisscFv, or a bi-specific T-cell engager (BiTE)), trispecific antibody (e.g., F(ab)′3 fragments or a triabody), or a chemically modified derivative thereof. In some embodiments, the anti-TfR antigen-binding protein can be bivalent. In some embodiments, the anti-TfR antigen-binding protein can be monovalent (e.g., one-arm antibody).

The term “humanized antibody,” as used herein, includes antibodies in which CDR sequences derived from the germline of another mammalian species, such as a mouse, have been grafted onto human framework sequences, or otherwise modified to increase their similarity to antibody variants produced naturally in humans.

In some cases, the anti-TfR antigen-binding protein is an antibody which comprises one or more mutations in a framework region, e.g., in the CH1 domain, CH2 domain, CH3 domain, hinge region, or a combination thereof. In some embodiments, the one or more mutations are to stabilize the antibody and/or to increase half-life. In some embodiments, the one or more mutations are to modulate Fc receptor interactions, to reduce or eliminate Fc effector functions such as FcγR, antibody-dependent cell-mediated cytotoxicity (ADCC), or complement-dependent cytotoxicity (CDC). In additional embodiments, the one or more mutations are to modulate glycosylation.

In some embodiments, one, two or more mutations (e.g., amino acid substitutions) are introduced into the Fc region of an antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity. In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., PCT Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo. In some embodiments, the Fc region comprises a mutation at residue position L234, L235, or a combination thereof. In some embodiments, the mutations comprise L234 and L235. In some embodiments, the mutations comprise L234A and L235A.

The anti-TfR antibodies and antigen-binding fragments described herein may be modified after translation, e.g., glycosylated.

For example, antibodies and antigen-binding fragments described herein may be glycosylated (e.g., N-glycosylated and/or O-glycosylated) or aglycosylated. Typically, antibodies and antigen-binding fragments are glycosylated at the conserved residue N297 of the IgG Fc domain. Some antibodies and fragments include one or more additional glycosylation sites in a variable region. In an embodiment, the glycosylation site is in the following context: FN₂₉₇S or YN₂₉₇S.

In an embodiment, said glycosylation is any one or more of three different N-glycan types: high mannose, complex and/or hybrid that are found on IgGs with their respective linkage. Complex and hybrid types exist with core fucosylation, addition of a fucose residue to the innermost N-acetylglucosamine, and without core fucosylation.

In some cases, the anti-TfR antigen-binding protein is an aglycosylated antibody, i.e., an antibody that does not comprise a glycosylation sequence that might interfere with a transglutamination reaction, for instance an antibody that does not have a saccharide group at N180 and/or N297 on one or more heavy chains. In particular embodiments, an antibody heavy chain has an N180 mutation. In other words, the antibody is mutated to no longer have an asparagine residue at position 180 according to the EU numbering system as disclosed by Kabat et al. In particular embodiments, an antibody heavy chain has an N180Q mutation. In particular embodiments, an antibody heavy chain has an N297 mutation. In particular embodiments, an antibody heavy chain has an N297Q or an N297D mutation. Antibodies comprising such above-described mutations can be prepared by site-directed mutagenesis to remove or disable a glycosylation sequence or by site-directed mutagenesis to insert a glutamine residue at site apart from any interfering glycosylation site or any other interfering structure. Such antibodies also can be isolated from natural or artificial sources. Aglycosylated antibodies also include antibodies comprising a T299 or S298P or other mutations, or combinations of mutations that result in a lack of glycosylation.

In some cases, the antigen-binding protein is a deglycosylated antibody, i.e., an antibody in which a saccharide group at is removed to facilitate transglutaminase-mediated conjugation. Saccharides include, but are not limited to, N-linked oligosaccharides. In some embodiments, deglycosylation is performed at residue N180. In some embodiments, deglycosylation is performed at residue N297. In some embodiments, removal of saccharide groups is accomplished enzymatically, included but not limited to via PNGase.

In an embodiment, an antibody or fragment described herein is afucosylated.

The antibodies and antigen-binding fragments described herein may also be post-translationally modified in other ways including, for example: Glu or Gln cyclization at N-terminus; Loss of positive N-terminal charge; Lys variants at C-terminus; Deamidation (Asn to Asp); Isomerization (Asp to isoAsp); Deamidation (Gln to Glu); Oxidation (Cys, His, Met, Tyr, Trp); and/or Disulfide bond heterogeneity (Shuffling, thioether and trisulfide formation).

In some embodiments, an antibody disclosed herein comprises Q295 which can be native to the antibody heavy chain sequence. In some embodiments, an antibody heavy chain disclosed herein may comprise Q295. In some embodiments, an antibody heavy chain disclosed herein may comprise Q295 and an amino acid substitution N297D.

According to certain embodiments of the present disclosure, anti-TfR antibodies and antigen-binding fragments are provided comprising an Fc domain comprising one or more mutations which enhance or diminish antibody binding to the FcRn receptor, e.g., at acidic pH as compared to neutral pH. For example, the present disclosure includes anti-TfR antibodies comprising a mutation in the CH2 or a CH3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal.

Non-limiting examples of such Fc modifications include, e.g., a modification at position:
250 (e.g., E or Q);
250 and 428 (e.g., L or F);
252 (e.g., L/Y/F/W or T),
254 (e.g., S or T), and/or
256 (e.g., S/R/Q/E/D or T);
   and/or a modification at position:
428 and/or 433 (e.g., H/L/R/S/P/Q or K), and/or
434 (e.g., A, W, H, F or Y);
   and/or a modification at position:
250 and/or 428;
   and/or a modification at position:
307 or 308 (e.g., 308F, V308F), and/or
434.

In an embodiment, the modification comprises:
a 428L (e.g., M428L) and 434S (e.g., N434S) modification;
a 428L, 259I (e.g., V259I), and 308F (e.g., V308F) modification;
a 433K (e.g., H433K) and a 434 (e.g., 434Y) modification;
a 252, 254, and 256 (e.g., 252Y, 254T, and 256E) modification;
a 250Q and 428L modification (e.g., T250Q and M428L); and/or
a 307 and/or 308 modification (e.g., 308F or 308P).

For example, the present disclosure includes anti-TfR antibodies comprising an Fc domain comprising one or more pairs or groups of mutations selected from the group consisting of:
250Q and 248L (e.g., T250Q and M248L);
252Y, 254T and 256E (e.g., M252Y, S254T and T256E);
2571 and 3111 (e.g., P2571 and Q311I);
2571 and 434H (e.g., P2571 and N434H);
376V and 434H (e.g., D376V and N434H);
307A, 380A and 434A (e.g., T307A, E380A and N434A);
428L and 434S (e.g., M428L and N434S); and
433K and 434F (e.g., H433K and N434F).

In yet another embodiment, the modification comprises a 265A (e.g., D265A) and/or a 297A (e.g., N297A) modification.

In an embodiment, the heavy chain constant domain is gamma4 comprising an S228P and/or S108P mutation. See Angal et al., A single amino acid substitution abolishes the heterogeneity of chimeric mouse/human (IgG4) antibody, Mol Immunol. 1993 January; 30(1):105-108.

All possible combinations of the foregoing Fc domain mutations, and other mutations within the antibody variable domains disclosed herein, are contemplated within the scope of the present disclosure.

The anti-TfR antibodies described herein may comprise a modified Fc domain having reduced effector function. As used herein, a “modified Fc domain having reduced effector function” means any Fc portion of an immunoglobulin that has been modified, mutated, truncated, etc., relative to a wild-type, naturally occurring Fc domain such that a molecule comprising the modified Fc exhibits a reduction in the severity or extent of at least one effect selected from the group consisting of cell killing (e.g., ADCC and/or CDC), complement activation, phagocytosis and opsonization, relative to a comparator molecule comprising the wild-type, naturally occurring version of the Fc portion. In certain embodiments, a “modified Fc domain having reduced effector function” is an Fc domain with reduced or attenuated binding to an Fc receptor (e.g., FcTR).

In certain embodiments, the modified Fc domain is a variant IgG1 Fc or a variant IgG4 Fc comprising a substitution in the hinge region. For example, a modified Fc for use in the context of the present disclosure may comprise a variant IgG1 Fc wherein at least one amino acid of the IgG1 Fc hinge region is replaced with the corresponding amino acid from the IgG2 Fc hinge region. Alternatively, a modified Fc for use in the context of the present disclosure may comprise a variant IgG4 Fc wherein at least one amino acid of the IgG4 Fc hinge region is replaced with the corresponding amino acid from the IgG2 Fc hinge region. Non-limiting, exemplary modified Fc regions that can be used in the context of the present disclosure are set forth in US Patent Application Publication No. 2014/0243504, the disclosure of which is hereby incorporated by reference in its entirety, as well as any functionally equivalent variants of the modified Fc regions set forth therein.

Also provided herein are antigen-binding proteins, antibodies or antigen-binding fragments, comprising a HCVR set forth herein and a chimeric heavy chain constant (CH) region, wherein the chimeric CH region comprises segments derived from the CH regions of more than one immunoglobulin isotype. For example, the antibodies of the disclosure may comprise a chimeric CH region comprising part or all of a CH2 domain derived from a human IgG1, human IgG2 or human IgG4 molecule, combined with part or all of a CH3 domain derived from a human IgG1, human IgG2 or human IgG4 molecule. According to certain embodiments, the antibodies provided herein comprise a chimeric CH region having a chimeric hinge region. For example, a chimeric hinge may comprise an “upper hinge” amino acid sequence (amino acid residues from positions 216 to 227 according to EU numbering) derived from a human IgG1, a human IgG2 or a human IgG4 hinge region, combined with a “lower hinge” sequence (amino acid residues from positions 228 to 236 according to EU numbering) derived from a human IgG1, a human IgG2 or a human IgG4 hinge region. According to certain embodiments, the chimeric hinge region comprises amino acid residues derived from a human IgG1 or a human IgG4 upper hinge and amino acid residues derived from a human IgG2 lower hinge. An antibody comprising a chimeric CH region as described herein may, in certain embodiments, exhibit modified Fe effector functions without adversely affecting the therapeutic or pharmacokinetic properties of the antibody. See, e.g., WO2014/022540.

Other modified Fc domains and Fc modifications that can be used in the context of the present disclosure include any of the modifications as set forth in US2014/0171623; U.S. Pat. No. 8,697,396; US2014/0134162; WO2014/043361, the disclosures of which are hereby incorporated by reference in their entireties. Methods of constructing antibodies or other antigen-binding fusion proteins comprising a modified Fc domain as described herein are known in the art.

In some embodiments, the anti-TfR antibodies and antigen-binding fragments described herein comprise an Fc domain comprising one or more mutations in the CH2 and/or CH3 regions that generate a separate TfR binding site.

In an embodiment, the CH2 region comprises one or more amino acid mutations, or a combination thereof, selected from the following: a) position 47 is Glu, Gly, Gln, Ser, Ala, Asn, Tyr, or Trp; position 49 is Ile, Val, Asp, Glu, Thr, Ala, or Tyr; position 56 is Asp, Pro, Met, Leu, Ala, Asn, or Phe; position 58 is Arg, Ser, Ala, or Gly; position 59 is Tyr, Trp, Arg, or Val; position 60 is Glu; position 61 is Trp or Tyr; position 62 is Gln, Tyr, His, Ile, Phe, Val, or Asp; and position 63 is Leu, Trp, Arg, Asn, Tyr, or Val; b) position 39 is Pro, Phe, Ala, Met, or Asp; position 40 is Gln, Pro, Arg, Lys, Ala, Ile, Leu, Glu, Asp, or Tyr; position 41 is Thr, Ser, Gly, Met, Val, Phe, Trp, or Leu; position 42 is Pro, Val, Ala, Thr, or Asp; position 43 is Pro, Val, or Phe; position 44 is Trp, Gln, Thr, or Glu; position 68 is Glu, Val, Thr, Leu, or Trp; position 70 is Tyr, His, Val, or Asp; position 71 is Thr, His, Gln, Arg, Asn, or Val; and position 72 is Tyr, Asn, Asp, Ser, or Pro; c) position 41 is Val or Asp; position 42 is Pro, Met, or Asp; position 43 is Pro or Trp; position 44 is Arg, Trp, Glu, or Thr; position 45 is Met, Tyr, or Trp; position 65 is Leu or Trp; position 66 is Thr, Val, Ile, or Lys; position 67 is Ser, Lys, Ala, or Leu; position 69 is His, Leu, or Pro; and position 73 is Val or Trp; or d) position 45 is Trp, Val, Ile, or Ala; position 47 is Trp or Gly; position 49 is Tyr, Arg, or Glu; position 95 is Ser, Arg, or Gln; position 97 is Val, Ser, or Phe; position 99 is Ile, Ser, or Trp; position 102 is Trp, Thr, Ser, Arg, or Asp; position 103 is Trp; and position 104 is Ser, Lys, Arg, or Val; wherein the substitutions and the positions are determined with reference to amino acids 4-113 of

(SEQ ID NO: 536)
PCPAPELLGGPSVFLFPPKPKDTLMISRTPEVTCVVVDVSHEDPEVKEN
WYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYP
SDIAVEWESNGQPENNYKTTPPVLDSDGSFFLYSKLTVDKSRWQQGNVF
SCSVMHEALHNHYTQKSLSLSPGK.

In an embodiment, the CH3 region comprises one or more amino acid mutations, or a combination thereof, selected from the following: position 153 is Trp, Leu, or Glu; position 157 is Tyr or Phe; position 159 is Thr; position 160 is Glu; position 161 is Trp; position 162 is Ser, Ala, Val, or Asn; position 163 is Ser or Asn; position 186 is Thr or Ser; position 188 is Glu or Ser; position 189 is Glu; and position 194 is Phe; or b) position 118 is Phe or Ile; position 119 is Asp, Glu, Gly, Ala, or Lys; position 120 is Tyr, Met, Leu, Ile, or Asp; position 122 is Thr or Ala; position 210 is Gly; position 211 is Phe; position 212 is His, Tyr, Ser, or Phe; and position 213 is Asp; wherein the substitutions and the positions are determined with reference to amino acids 114-220 of SEQ ID NO: 536.

In some embodiments, the CH3 region comprises one or more mutations, or a combination thereof, selected from the following: position 384 is Leu, Tyr, Met, or Val; position 386 is Leu, Thr, His, or Pro; position 387 is Val, Pro, or an acidic amino acid; position 388 is Trp; position 389 is Val, Ser, or Ala; position 413 is Glu, Ala, Ser, Leu, Thr, or Pro; position 416 is Thr or an acidic amino acid; and position 421 is Trp, Tyr, His, or Phe, according to EU numbering. In an embodiment, the CH3 region comprises one or more amino acid mutations, or a combination thereof, selected from the following: a) position 380 is Trp, Leu, or Glu; position 384 is Tyr or Phe; position 386 is Thr; position 387 is Glu; position 388 is Trp; position 389 is Ser, Ala, Val, or Asn; position 390 is Ser or Asn; position 413 is Thr or Ser; position 415 is Glu or Ser; position 416 is Glu; and position 421 is Phe.

In some embodiments, the CH3 region comprises one or more mutations, or a combination thereof, selected from the following: a) Phe at position 382, Tyr at position 383, Asp at position 384, Asp at position 385, Ser at position 386, Lys at position 387, Leu at position 388, Thr at position 389, Pro at position 419, Arg at position 420, Gly at position 421, Leu at position 422, Ala at position 424, Glu at position 426, Tyr at position 438, Leu at position 440, Gly at position 442, and Glu at position 443; b) Phe at position 382, Tyr at position 383, Gly at position 384, N at position 385, Ala at position 386, Lys at position 387, Thr at position 389, Leu at position 422, Ala at position 424, Glu at position 426, Tyr at position 438, Leu at position 440; c) Phe at position 382, Tyr at position 383, Glu at position 384, Ala at position 385, Lys at position 387, Leu at position 388, Leu at position 422, Ala at position 424, Glu at position 426, Tyr at position 438, Leu at position 440; d) Phe at position 382, Glu at position 384, Ser at position 386, Lys at position 387, Thr at position 389, Leu at position 422, Ala at position 424, Glu at position 426, Tyr at position 438, Leu at position 440; e) Phe at position 382, Gly at position 384, Ala at position 385, Lys at position 387, Ser at position 389, Leu at position 422, Ala at position 424, Glu at position 426, Tyr at position 438, Leu at position 440; f) Phe at position 382, Gly at position 384, Ala at position 385, Lys at position 387, Leu at position 388, Thr at position 389, Leu at position 422, Ala at position 424, Glu at position 426, Tyr at position 438, Leu at position 440; wherein the positions are determined according to EU numbering.

Additional mutations in CH2 and/or CH3 regions that can introduce non-native TfR binding sites into the antigen-binding proteins descried herein include those described in US Patent Application Publication Nos. 2020/0223935, 2020/0369746, 2021/0130485, 2022/0017634; and PCT application Publications Nos. WO2023/279099, WO2023/114499 and WO2023/114510, which are incorporated herein by reference in their entireties.

Provided herein is a vessel (e.g., a plastic or glass vial, e.g., with a cap or a chromatography column, hollow bore needle or a syringe cylinder) comprising an anti-TfR:Payload fusion protein, e.g., anti-TfR scFv:GAA or anti-TfR Fab:GAA, provided herein, e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263.

Also provided is an injection device comprising an anti-TfR:Payload fusion protein, e.g., anti-TfR scFv:GAA or anti-TfR Fab:GAA disclosed herein, e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263, or a pharmaceutical composition thereof. The injection device may be packaged into a kit. An injection device is a device that introduces a substance into the body of a subject via a parenteral route, e.g., intramuscular, subcutaneous or intravenous. For example, an injection device may be a syringe (e.g., pre-filled with the pharmaceutical composition, such as an auto-injector) which, for example, includes a cylinder or barrel for holding fluid to be injected (e.g., comprising the fusion protein or a pharmaceutical composition thereof), a needle for piercing skin and/or blood vessels for injection of the fluid; and a plunger for pushing the fluid out of the cylinder and through the needle bore.

Further provided are methods for administering an anti-TfR:Payload fusion protein, e.g., anti-TfR scFv:GAA or anti-TfR Fab:GAA provided herein, e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263, to a subject, comprising introducing the fusion protein into the body of the subject (e.g., a human), for example, parenterally. For example, the method comprises piercing the body of the subject with a needle of a syringe and injecting the fusion protein into the body of the subject, e.g., into the vein, artery, tumor, muscular tissue or subcutis of the subject.

Further provided herein are methods for delivering a payload wherein the payload is fused to, e.g., an antigen-binding protein provided herein, e.g., anti-TfR scFv:GAA or anti-TfR Fab:GAA provided herein, e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263, to a targeted tissue in a subject (e.g., any of the tissues or cell types or cell types in or associated with the corresponding tissues as set forth in Table C below; or brain, cerebral cortex; cerebellum; hippocampus; caudate; parathyroid gland; adrenal gland; bronchus; lung; oral mucosa; esophagus; stomach; duodenum; small intestine; colon; rectum; liver; gallbladder; pancreas; kidney; urinary bladder; testis; epididymis; prostate; vagina; ovary; fallopian tube; endometrium; cervix; placenta; breast; muscle, (e.g., heart muscle; skeletal muscle, smooth muscle and/or endothelial vasculature thereof); soft tissue; skin; appendix; lymph node; tonsil; and/or bone marrow), comprising introducing the fusion protein into the body of the subject (e.g., a human), for example, parenterally. For example, the method comprises piercing the body of the subject with a needle of a syringe and injecting the fusion protein into the body of the subject, e.g., into the vein, artery, tumor, muscular tissue or subcutis of the subject.

TABLE C
Tissue And Cell Types Which Can Be Targeted
For Delivery Of A Payload Using An Anti-Tfr.

| Target tissue | Cell types |
|---|---|
| Brain/Spinal cord/CNS | endothelial cells |
| | neurons (all types) |
| | oligodendrocytes (and precursors) |
| | pericytes |
| | meninges/leptomeningeal cells |
| | arachnoid barrier cells |
| | peripheral glia |
| | astrocytes |
| | glia |
| | Schwann cells |
| | ependymal cells |
| | microglia |
| Eye | rod photoreceptor cells |
| | Muller glia cells |
| | bipolar cells |
| | cone photoreceptor cells |
| | endothelial cells |
| | cornea |
| | sclera |
| | optic nerve |
| | pupillary sphincter |
| Skeletal Muscle | skeletal myocytes |
| | fibroblasts |
| | endothelial cells |
| | macrophages |
| | satellite cells |
| Adipose tissue | adipocytes |
| | fibroblasts |
| | T-cells |
| | macrophages |
| | B-cells |
| | dendritic cells |
| Blood/Bone marrow | T-cells |
| | B-cells |
| | macrophages |
| | erythroid cells |
| | plasmid cells |
| | dendritic cells |
| Breast | glandular cells |
| | T-cells |
| | fibroblasts |
| | macrophages |
| | endothelial cells |
| | myoepithelial cells |
| | adipocytes |
| Lung/Bronchus | basal respiratory cells |
| | respiratory ciliated cells |
| | club cells |
| | smooth muscle cells |
| | ionocytes |
| | macrophages |
| | alveolar cells (type 1 & 2) |
| | T-cells |
| | endothelial cells |
| Colon | distal enterocytes |
| | intestinal goblet cells |
| | undifferentiated cells |
| | T-cells |
| | Paneth cells |
| | B-cells |
| | enteroendocrine cells |
| Uterus | glandular and luminal cells |
| | endometrial stromal cells |
| | endothelial cells |
| | smooth muscle cells |
| | T-cells |
| | macrophages |
| Esophagus | fibroblasts |
| | squamous epithelial cells |
| | endothelial cells |
| | smooth muscle cells |
| | macrophages |
| | plasma cells |
| | T-cells |
| Heart | cardiomyocytes |
| | endothelial cells |
| | fibroblasts |
| | macrophages |
| | T-cells |
| | B-cells |
| | dendritic cells |
| Kidney | proximal tubular cells |
| | T-cells |
| | macrophages |
| | collecting duct cells |
| | B-cells |
| | glomeruli |
| | fibroblasts |
| Liver | hepatocytes |
| | B-cells |
| | erythroid cells |
| Lymph node | B-cells |
| | T-cells |
| Ovary | granulosa cells |
| | fibroblasts |
| | smooth muscle cells |
| | macrophages |
| | T-cells |
| | theca cells |
| | fibroblasts |
| Pancreas | ductal cells |
| | pancreatic endocrine cells |
| | smooth muscle cells |
| | endothelial cells |
| | macrophages |
| | exocrine glandular cells |
| | monocytes |
| Placenta | cytotrophoblasts |
| | extravillous trophoblasts |
| | fibroblasts |
| | Hofbauer cells |
| | endothelial cells |
| Prostate | basal prostatic cells |
| | prostatic glandular cells |
| | urothelial cells |
| | endothelial cells |
| | fibroblasts |
| | smooth muscle cells |
| | macrophages |
| | T-cells |
| Rectum | undifferentiated cells |
| | intestinal goblet cells |
| | Paneth cells |
| | distal enterocytes |
| | enteroendocrine cells |
| Skin | Langerhans cells |
| | fibroblasts |
| | endothelial cells |
| | basal keratinocytes |
| | suprabasal keratinocytes |
| | T-cells |
| | smooth muscle cells |
| | melanocytes |
| PBMC | monocytes |
| | T-cells |
| | NK-cells |
| | dendritic cells |
| Small intestine | proximal enterocytes |
| | undifferentiated cells |
| | intestinal goblet cells |
| | Paneth cells |
| Spleen | B-cells |
| | T-cells |
| | plasma cells |
| | macrophages |
| Stomach | B-cells |
| | T-cells |
| | gastric mucus-secreting cells |
| | plasma cells |
| | fibroblasts |
| | macrophages |
| Testis | Leydig cells |
| | late spermatids |
| | spermatogonia |
| | early spermatids |
| | macrophages |
| | spermatocytes |
| | peritubular cells |
| | Sertoli cells |
| | endothelial cells |
| Peripheral nervous system | motor neurons |
| | sensory neurons |
| | Schwann cells |
| | dorsal root ganglion |
| Bone/cartilage/joint | chondrocytes |
| | chondroblasts |
| | mesenchymal cells |
| | osteoblasts |
| | osteoclasts |

### III. Treatment and Administration

Provided are anti-TfR:Payload fusion proteins (e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; and 69263), or polynucleotides encoding anti-TfR Payload fusion proteins, wherein the payload is a GAA polypeptide or variant thereof, which can be used, for example, for delivering GAA peptide to the body of a subject, e.g., for treating or preventing a disease or disorder mediated, at least in part, by deficiency of GAA protein and/or activity in the body (e.g., the brain) of the subject (a Glycogen Storage Disease (GSD)). Pompe disease is an example of a GSD. For example, a GSD is a glycogen storage disease that is mediated by deficiency in GAA.

Glycogen storage disease (GSD) type 2, also known as Pompe disease or acid maltase deficiency disease, is an example of GSD which is an inherited metabolic disorder. While glycogen storage disease type 2 is a single disease, it may be classified in 2 forms according to the rates of disease progression, its severity and the age at which symptoms start. The classic infantile-onset starts before 12 month of age and involves the heart muscle (myocardiopathy). The later-onset form may start before 12 months of age (non-classic infantile-onset), or after 12 months of age, but does not affect the heart. Muscle weakness is a main symptom in all forms. The infantile-onset is the most severe form and, if untreated, it may lead to death from heart failure in the first year of life. The late-onset form is usually milder, but if untreated may lead to severe breathing problems.

Glycogen storage disease type 2 is caused by variants (mutations) in the GAA gene which have instructions to produce the enzyme acid alpha-glucosidase (acid maltase), needed to break down glycogen, a substance that is a source of energy for the body. The enzyme deficiency results in the accumulation of glycogen inside lysosomes, structures within cells that break down waste products within the cell. Accumulation of glycogen in certain tissues, especially muscles, impairs their function.

The classic infantile form of glycogen storage disease type 2 is characterized by severe muscle weakness (myopathy) and abnormally diminished muscle tone (hypotonia) without muscle wasting, and usually manifests within the first few months of life. Additional abnormalities may include enlargement of the heart (cardiomegaly), the liver (hepatomegaly), and/or the tongue (macroglossia). Affected infants may also have poor feeding, failure to gain weight and grow at the expected rate (failure to thrive), breathing problems, and hearing loss. Most infants with glycogen storage disease type 2 cannot hold up their heads or move normally. Without treatment, progressive cardiac failure usually causes life-threatening complications by the age of 12 to 18 months.

The non-classic infantile form of glycogen storage disease type 2 usually presents within the first year of life. Initial symptoms may include delayed motor skills (crawling, sitting) and myopathy. Cardiomegaly may be present, but unlike the classic infantile form, cardiac failure does not typically occur. Muscle weakness may lead to serious, life-compromising breathing problems by early childhood.

In the late onset form of glycogen storage disease type 2, symptoms may not be evident until childhood, adolescence, or adulthood. This form is usually milder than the infantile-onset form of the disorder. Most individuals experience progressive muscle weakness, especially in the legs and the trunk, including the muscles that control breathing.

Thus, provided herein are methods for treating or preventing a glycogen storage disease (e.g., the classic infantile form, the non-classic infantile form or the late onset form of glycogen storage disease type 2), in a subject in need thereof, by administering a therapeutically effective amount of anti-TfR:GAA fusion protein (e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; and 69263) or polynucleotides encoding anti-TfR Payload fusion proteins, to the subject, e.g., wherein one or more signs or symptoms of the GSD are alleviated.

In an embodiment, a subject having Pompe disease has one or more of the following GAA mutations (e.g., homozygous or heterozygous):
ASP91ASN
MET318THR
GLU521LYS
GLY643ARG
ARG725TRP
IVS1AS, T-G, −13
   T-to-G transversion at position −13 of the acceptor site of intron 1 of the GAA gene, resulting in alternatively spliced transcripts with deletion of the first coding exon, exon 2. Huie et al., Aberrant splicing in adult onset glycogen storage disease type II (GSDII): molecular identification of an IVS1 (−13T to G) mutation in a majority of patients and a novel IVS10 (+GT to CT) mutation. Hum. Molec. Genet. 3: 2231-2236, 1994.
LYS903DEL
LEU299ARG
SER529VAL
ASP645GLU
GLU689LYS
EX18DEL
   Deletion of exon 18 of the GAA gene. Van der Kraan et al., Deletion of exon 18 is a frequent mutation in glycogen storage disease type II. Biochem. Biophys. Res. Commun. 203: 1535-1541, 1994
PRO545LEU
1-BP DEL, 525T
   Two mutations in the GAA gene: P545L and a 1-bp deletion (525delT), resulting in premature termination of the protein at nucleotide positions 658 to 660. Hermans et al., The effect of a single base pair deletion (delta-T525) and a C1634T missense mutation (pro5451eu) on the expression of lysosomal alpha-glucosidase in patients with glycogen storage disease type II. Hum. Molec. Genet. 3: 2213-2218, 1994
ARG854TER (nonsense mutation)
ALA237VAL
GLY293ARG
IVS6AS, G-C, −1
   G-to-C transversion in intron 6 of the GAA gene (1076-1G-C). Gort et al., A. Glycogen storage disease type II in Spanish patients: high frequency of c.1076-1G-C mutation. Molec. Genet. Metab. 92: 183-187, 2007.

Thus, provided herein are methods for treating or preventing a GSD (e.g., the classic infantile form, the non-classic infantile form or the late onset form of glycogen storage disease type 2), in a subject in need thereof, wherein the subject has GAA comprising one or more of said mutations, by administering a therapeutically effective amount of anti-TfR:GAA fusion protein (e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; and 69263) or polynucleotides encoding anti-TfR Payload fusion proteins, to the subject, e.g., wherein one or more signs or symptoms of the GSD are alleviated.

As used herein, the term “subject” refers to a mammal (e.g., rat, mouse, cat, dog, cow, sheep, horse, goat, rabbit), preferably a human, for example, in need of prevention and/or treatment of a GAA-deficiency disease or disorder. In an embodiment, a subject has been diagnosed as suffering from a GSD such as Pompe Disease.

Provided herein are combinations including an anti-TfR:Payload fusion protein provided herein (e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; and 69263) or polynucleotides encoding anti-TfR Payload fusion proteins, in association with one or more further therapeutic agents. The anti-TfR:Payload fusion protein and the further therapeutic agent can be in a single composition or in separate compositions. For example, in an embodiment, the further therapeutic agent is alglucosidase alfa (e.g., Myozyme or Lumizyme), Rituximab, Methotrexate, Intravenous immunoglobulin (IVIG), avalglucosidase alfa-ngpt (e.g., Nexviazyme), a selective beta agonist (e.g., levalbuterol), an antibiotic, a steroid (e.g., cortisone or prednisone), a bisphosphonate, an infectious disease treatment (e.g., an antibiotic, a vaccine (e.g., Pneumococcal vaccine), palivizumab).

Methods for treating or preventing a GSD (e.g., Pompe Disease) in a subject in need of said treatment or prevention by administering an anti-TfR:GAA fusion protein, e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; and 69263, or polynucleotides encoding anti-TfR Payload fusion proteins, in association with a further therapeutic agent are provided herein. Compositions comprising the anti-TfR:GAA fusion protein in association with one or more further therapeutic agents are also provided herein.

The term “in association with” indicates that components, an anti-TfR:Payload fusion protein provided herein, along with another agent such as methotrexate, can be formulated into a single composition, e.g., for simultaneous delivery, or formulated separately into two or more compositions (e.g., a kit including each component). Each component can be administered to a subject at a different time than when the other component is administered; for example, each administration may be given non-simultaneously (e.g., separately or sequentially) at intervals over a given period of time. Moreover, the separate components may be administered to a subject by the same or by a different route.

An effective or therapeutically effective dose of anti-TfR:Payload fusion protein provided herein for treating or preventing a GAA-deficiency disease or disorder refers to the amount of anti-TfR:Payload sufficient to alleviate one or more signs and/or symptoms of the disease or condition in the treated subject, whether by inducing the regression or elimination of such signs and/or symptoms or by inhibiting the progression of such signs and/or symptoms. In an embodiment, an effective or therapeutically effective dose of anti-TfR:GAA is about 1 mg/kg to about 50 mg/kg. The dose amount may vary depending upon the age and the size of a subject to be administered, target disease, conditions, route of administration, and the like. In certain embodiments, the initial dose may be followed by administration of a second or a plurality of subsequent doses of antigen-binding protein in an amount that can be approximately the same or less or more than that of the initial dose, wherein the subsequent doses are separated by days or weeks.

The diagnosis of Pompe disease can be based on a thorough clinical evaluation, a detailed patient and family history, and a variety of biochemical tests including the measuring of GAA activity. In individuals suspected of having Pompe disease, blood can be drawn and the function/activity of GAA can be measured in white blood cells (leukocytes). Proper assay conditions should be used and acarbose should be added to the reaction mixture to inhibit the activity of glucoamylase. Alternatively, the GAA activity/functional assay can also be performed on dried blood spots, although this method is not any quicker, less reliable, and also requires the use of acarbose to inhibit the glucoamylase activity.

Each diagnosis performed with the dried blood spot test method should be confirmed through molecular genetic testing (GAA gene copy analysis) or by measuring the GAA activity with another method. Leukocytes can be used for this purpose, but cultured skin fibroblasts obtained by a skin biopsy are the very best material. More invasive muscle biopsies are not needed and not optimal either for measuring the GAA activity.

The application of a skin biopsy and the initiation of a culture of skin fibroblasts might not be feasible in every diagnostic setting, but should be considered as there are important advantages with this procedure. The GAA activity/functional test using skin fibroblasts is superior to all other methods for its high sensitivity and for discriminating between classic-infantile, childhood and adult Pompe disease (IOPD vs LOPD) in almost all cases.

A variety of other tests can be performed to detect or assess symptoms potentially associated with Pompe disease such as sleep studies, tests that measure lung function, and tests that measure muscle function. Muscle MRI (imaging by magnetic resonance) is used to visualize the degree of muscle damage.

Specific tests may also be performed to assess the heart function, including chest x-ray, electrocardiography (ECG), and echocardiography (imaging by ultrasound). Chest x-rays allow physicians to assess the size of the heart, which is enlarged in classic infantile Pompe disease. Electrocardiography (ECG) measures the electric activity of the heart and detects abnormal heart rhythms. Echocardiography uses reflected sound waves to create a picture of the heart and can reveal abnormal thickening of the walls of the heart.

### IV. Payloads

The anti-TfR antigen-binding proteins set forth herein are useful for delivering any of many types of payload to a targeted tissue (e.g., brain)—for example, therapeutic agents (TAs). Such payloads include proteins, enzymes and viral vectors containing polynucleotides. The delivery of any payload by fusion to an anti-TfR antigen-binding protein may be referred to as anti-TfR-mediated delivery.

Payloads include polypeptides, e.g., enzymes and antigen-binding proteins (e.g., antibodies and antigen-binding fragments thereof). In some embodiments, the enzyme is a hydrolase, including esterases, glycosylases, hydrolases that act on ether bonds, peptidases, linear amidases, diphosphatases, ketone hydrolases, halogenases, phosphoamidases, sulfohydrolases, sulfinases, desulfinases, and the like. In some embodiments, the enzyme is a glycosylase, including glycosidases and N-glycosylases. In some embodiments, the enzyme is a glycosidase, including alpha-amylase, beta-amylase, glucan 1,4-alpha-glucosidase, cellulose, endo-1,3(4)-beta-glucanase, inulinase, endo-1,4-beta-xylanase, endo-1,4-b-xylanase, dextranase, chitinase, polygalacturonidase, lysozyme, exo-alpha-sialidase, alpha-glucosidase, beta-glucosidase, alpha-galactosidase, beta-galactosidase, alpha-mannosidase, beta-mannosidase, beta-fructofuranosidase, alpha,alpha-trehalose, beta-glucuronidase, xylan endo-1,3-beta-xylosidase, amylo-alpha-1,6-glucosidase, hyaluronoglucosaminidase, hyaluronoglucuronidase, and the like.

In some embodiments, the payload is a alpha-glucosidase (GAA) polypeptide. GAA is described in more detail elsewhere herein.

In some embodiments, the payload is an alpha-galactosidase A (GLA) polypeptide. “Alpha-galactosidase A” (GLA or “α-galactosidase A”) facilitates the hydrolysis of terminal α-galactosyl moieties from glycolipids and glycoproteins, and also hydrolyses α-D-fucosides. GLA is also known inter alia as EC 3.2.1.22, melibiase, α-D-galactosidase, α-galactosidase A, α-galactoside galactohydrolase, α-D-galactoside galactohydrolase. Fabry disease is caused by defective lysosomal enzyme alpha-galactosidase A (GLA), which results in the accumulation of globotriaosylceramide within the blood vessels and other tissues and organs. Symptoms associated with Fabry disease include pain from nerve damage and/or small vascular obstruction, renal insufficiency and eventual failure, cardiac complications such as high blood pressure and cardiomyopathy, dermatological symptoms such as formation of angiokeratomas, anhidrosis or hyperhidrosis, and ocular problems such as cornea verticillata, spoke-like cataract, and conjunctival and retinal vascular abnormalities. Treatments include FABRAZYME (agalsidase beta), REPLAGAL (agalsidase alfa) and GALAFOLD. Thus, provided herein are anti-TfR:Payload fusion proteins wherein the payload is alpha-galactosidase A, agalsidase beta, agalsidase alfa or miglastat as well as methods for treating Fabry disease in a patient by administering an effective amount of such a fusion protein to the patient.

In some embodiments, the payload is an acid sphingomyelinase (ASM) polypeptide. “Acid sphingomyelinase” (ASM, sphingomyelin phosphodiesterase, or SMPD1) converts sphingomyelin to ceramide. ASMD (acid sphingomyelinase deficiency) is historically known as Niemann-Pick disease types A, A/B, and B. In people with ASMD, the body is unable to make enough of the ASM enzyme, and sphingomyelin cannot be broken down efficiently, and instead builds up in major organs such as the liver, lungs, and spleen. This can lead to complications over time, as key organs in the body may not be able to function properly. Niemann-Pick disease A (NPDA) is an early-onset lysosomal storage disorder caused by failure to hydrolyze sphingomyelin to ceramide. It results in the accumulation of sphingomyelin and other metabolically related lipids in reticuloendothelial and other cell types throughout the body, leading to cell death. Niemann-Pick disease type A is a primarily neurodegenerative disorder characterized by onset within the first year of life, intellectual disability, digestive disorders, failure to thrive, major hepatosplenomegaly, and severe neurologic symptoms. The severe neurological disorders and pulmonary infections lead to an early death, often around the age of four. Clinical features are variable. A phenotypic continuum exists between type A (basic neurovisceral) and type B (purely visceral) forms of Niemann-Pick disease, and the intermediate types encompass a cluster of variants combining clinical features of both types A and B. Niemann-Pick disease B (NPDB) is a late-onset lysosomal storage disorder caused by failure to hydrolyze sphingomyelin to ceramide. It results in the accumulation of sphingomyelin and other metabolically related lipids in reticuloendothelial and other cell types throughout the body, leading to cell death. Clinical signs involve only visceral organs. The most constant sign is hepatosplenomegaly which can be associated with pulmonary symptoms. Patients remain free of neurologic manifestations. However, a phenotypic continuum exists between type A (basic neurovisceral) and type B (purely visceral) forms of Niemann-Pick disease, and the intermediate types encompass a cluster of variants combining clinical features of both types A and B. In Niemann-Pick disease type B, onset of the first symptoms occurs in early childhood and patients can survive into adulthood.

In some embodiments, the payload is a lysosomal acid glucosylceramidase (GBA) polypeptide. “Lysosomal acid glucosylceramidase” (GBA, glucocerebrosidase, lysosomal acid GCase, acid beta-glucosidase, alglucerase, beta-glucocerebrosidase, beta-GC, beta-glucosylceramidase 1, cholesterol glucosyltransferase, cholesteryl-beta-glucosidase, D-glucosyl-N-acylsphingosine glucohydrolase, glucosylceramidase beta 1, imiglucerase, lysosomal cholesterol glycosyltransferase, lysosomal galactosylceramidase, lysosomal glycosylceramidase, GBA, GBA1, GC, or GLUC) hydrolyzes glucosylceramide (GlcCer) to glucose and ceramide. In addition, GCase catalyzes the transfer of glucose from GlcCer to cholesterol to contribute to in the synthesis of 0-cholesteryl glucoside. Homozygous GBA mutations result in the most common lysosomal storage disorder, Gaucher disease (GD), which is classified according to the presence (neuronopathic types, type 2 and 3 GD) or absence (non-neuronopathic type, type 1 GD) of neurological symptoms.

A. Alpha-Glucosidase (GAA) Payload

Provided herein are methods and compositions for delivering the payload, alpha-glucosidase (GAA) mature peptide, preferably human GAA, to the brain. Alpha-glucosidases are enzymes that catalyze the exohydrolysis of 1,4-alpha-glucosidic linkages with release of alpha-glucose. Preferably, the TfR to which an antigen-binding protein (e.g., scFv) binds is from the same species from which the GAA polypeptide is obtained; for example, anti-human TfR is fused to a human GAA (or a variant thereof).

“Acid alpha-glucosidase” or “alpha-glucosidase” or “GAA” is intended to refer to the mature peptide of the human enzyme. The enzyme hydrolyzes alpha-1,4 linkages between the D-glucose units of glycogen, maltose, and isomaltose. Alternative names include but are not limited to lysosomal alpha-glucosidase (EC:3.2.1.20); glucoamylase; 1,4-alpha-D-glucan glucohydrolase; amyloglucosidase; gamma-amylase and exo-1,4-alpha-glucosidase. Human acid alpha-glucosidase is encoded by the GAA gene (National Centre for Biotechnology Information (NCBI) Gene ID 2548), which has been mapped to the long arm of chromosome 17 (location 17q25.2-q25.3). More than 500 mutations have currently been identified in the human GAA gene, many of which are associated with Pompe disease. Mutations resulting in misfolding or misprocessing of the acid alpha-glucosidase enzyme include T1064C (Leu355Pro) and C2104T (Arg702Cys). In addition, GAA mutations which affect maturation and processing of the enzyme include Leu405Pro and Met519Thr. The conserved hexapeptide WIDMNE at amino acid residues 516-521 is required for activity of the acid alpha-glucosidase protein. As used herein, the abbreviation “GAA” is intended to refer to the acid alpha-glucosidase enzyme, while the italicized abbreviation “GAA” is intended to refer to the human gene coding for the human acid alpha-glucosidase enzyme.

In an embodiment, the mature peptide of human alpha-glucosidase comprises the amino acid sequence:

(SEQ ID NO: 325)
AHPGRPRAVPTQCDVPPNSREDCAPDKAITQEQCEARGCCYIPAKQGLQ
GAQMGQPWCFFPPSYPSYKLENLSSSEMGYTATLTRTTPTFFPKDILTL
RLDVMMETENRLHFTIKDPANRRYEVPLETPHVHSRAPSPLYSVEFSEE
PFGVIVRRQLDGRVLLNTTVAPLFFADQFLQLSTSLPSQYITGLAEHLS
PLMLSTSWTRITLWNRDLAPTPGANLYGSHPFYLALEDGGSAHGVFLLN
SNAMDVVLQPSPALSWRSTGGILDVYIFLGPEPKSVVQQYLDVVGYPFM
PPYWGLGFHLCRWGYSSTAITRQVVENMTRAHFPLDVQWNDLDYMDSRR
DFTENKDGFRDFPAMVQELHQGGRRYMMIVDPAISSSGPAGSYRPYDEG
LRRGVFITNETGQPLIGKVWPGSTAFPDFTNPTALAWWEDMVAEFHDQV
PFDGMWIDMNEPSNFIRGSEDGCPNNELENPPYVPGVVGGTLQAATICA
SSHQFLSTHYNLHNLYGLTEAIASHRALVKARGTRPFVISRSTFAGHGR
YAGHWTGDVWSSWEQLASSVPEILQFNLLGVPLVGADVCGFLGNTSEEL
CVRWTQLGAFYPFMRNHNSLLSLPQEPYSFSEPAQQAMRKALTLRYALL
PHLYTLFHQAHVAGETVARPLFLEFPKDSSTWTVDHQLLWGEALLITPV
LQAGKAEVTGYFPLGTWYDLQTVPVEALGSLPPPPAAPREPAIHSEGQW
VTLPAPLDTINVHLRAGYIIPLQGPGLTTTESRQQPMALAVALTKGGEA
RGELFWDDGESLEVLERGAYTQVIFLARNNTIVNELVRVTSEGAGLQLQ
KVTVLGVATAPQQVLSNGVPVSNFTYSPDTKVLDICVSLLMGEQFLVSW
C

### V. Lysosomal Storage Diseases and Disorders

Provided herein are anti-TfR:Payload fusion proteins wherein the payload is a lysosomal storage disease therapeutic agent (LSD-TA), e.g., an LSD protein (which may be referred to as an anti-TfR:LSD protein fusion protein or anti-TfR:LSD protein fusion); e.g., wherein the antigen-binding protein of the fusion (e.g., scFv) is 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263. Methods for treating or preventing an LSD in a patient by administering an effective amount of anti-TfR:LSD-TA to the patient.

An LSD-therapeutic agent in an agent that, when delivered to a subject having an LSD, can treat such a disease. An LSD protein is any protein, e.g., an enzyme, that, when delivered to the cells of a patient having an LSD, treats or prevents the LSD. Preferably, the LSD protein is the enzyme for which the patient's lysosomes are deficient.

“Lysosomal storage diseases” (LSDs) include any disorder resulting from a defect in lysosome function. The most well-known lysosomal disease includes Tay-Sachs, Gaucher, and Niemann-Pick disease. The pathogeneses of the diseases are ascribed to the buildup of incomplete degradation products in the lysosome, sometimes due to loss of protein function. Lysosomal storage diseases may be caused by loss-of-function or attenuating variants in the proteins whose normal function is to degrade or coordinate degradation of lysosomal contents. The proteins affiliated with lysosomal storage diseases include enzymes, receptors and other transmembrane proteins (e.g., NPC1), post-translational modifying proteins (e.g., sulfatase), membrane transport proteins, and non-enzymatic cofactors and other soluble proteins (e.g., GM2 ganglioside activator). Thus, lysosomal storage diseases encompass more than those disorders caused by defective enzymes per se, and include any disorder caused by any molecular defect.

LSDs include sphingolipidoses (heterogeneous group of inherited disorders of lipid metabolism affecting primarily the central nervous system), a mucopolysaccharidoses (a group of inherited lysosomal storage disorders), and glycogen storage diseases. In some embodiments, the LSD is any one or more of Fabry disease, Gaucher disease type I, Gaucher disease type II, Gaucher disease type III, Niemann-Pick disease type A, Niemann-Pick disease type BGM1-gangliosidosis, Sandhoff disease, Tay-Sachs disease, GM2-activator deficiency, GM3-gangliosidosis, metachromatic leukodystrophy, sphingolipid-activator deficiency, Scheie disease, Hurler-Sceie disease, Hurler disease, Hunter disease, Sanfilippo A, Sanfilippo B, Sanfilippo C, Sanfilippo D, Morquio syndrome A, Morquio syndrome B, Maroteaux-Lamy disease, Sly disease, MPS IX, and Pompe disease. In a specific embodiment, the LSD is Fabry disease. In another embodiment, the LSD is Pompe disease. Thus, provided herein are methods for treating or preventing any such LSD in a patient by administering an effective amount of anti-TfR:LSD-TA to the patient.

The nature of the molecular lesion in a lysosomal storage disease affects the severity of the disease in many cases, i.e., complete loss-of-function may be associated with pre-natal or neo-natal onset, and involves severe symptoms; partial loss-of-function may be associated with milder (relatively) and later-onset disease. Only a small percentage of activity may need to be restored to have to correct metabolic defects in deficient cells. Table D-1 and D-2 lists some lysosomal storage diseases and their associated loss-of-function proteins. Lysosomal storage diseases are generally described in Desnick & Schuchman, “Enzyme replacement therapy for lysosomal diseases: lessons from 20 years of experience and remaining challenges,” 13 Annu. Rev. Genomics Hum. Genet. 307-35, 2012).

TABLE D-1
LSDs and corresponding Proteins which can be fused to an anti-TfR for treatment

| Class | LSD | LSD Protein |
|---|---|---|
| Sphingolipidoses | Fabry disease | α-Galactosidase A |
| | Farber lipogranulomatosis | Ceramidase |
| | Gaucher disease type I | β-Glucosidase |
| | Gaucher disease types II and III | Saposin-C activator |
| | Niemann-Pick diseases types A and B | Acid sphingomyelinase |
| | GM1-gangliosidosis | β-Galactosidase |
| | GM2-gangliosidosis (Sandhoff) | β-Hexosaminidase A and B |
| | GM2-gangliosidosis (Tay-Sachs) | β-Hexosaminidase A |
| | GM2-gangliosidosis (GM2-activator deficiency) | GM2-activator protein |
| | GM3-gangliosidosis | GM3 synthase |
| | Metachromatic leukodystrophy | Arylsulfatase A |
| | Sphingolipid-activator deficiency | Sphingolipid activator |
| Mucopoly- | MPS I (Scheie, Hurler-Scheie, and Hurler disease) | α-Iduronidase |
| saccharidoses | MPS II (Hunter) | Iduronidase-2-sulphatase |
| | MPS IIIA (Sanfilippo A) | Heparan N-sulphatase |
| | MPS IIIB (Sanfilippo B) | N-acetyl-α-glucosaminidase |
| | MPS IIIC (Sanfilippo C) | Acetyl-CoA; α-glucosamide N- |
| | | acetyltransferase |
| | MPS IIID (Sanfilippo D) | N-acetylglucosamine-6-sulphatase |
| | MPS IVA (Morquio syndrome A) | N-acetylgalactosamine-6-sulphate |
| | | sulphatase |
| | MPS IVB (Morquio syndrome B) | β-Galactosidase |
| | MPS VI (Maroteaux-Lamy) | N-acetylgalactosamine-4- |
| | | sulphatase (arylsulphatase B) |
| | MPS VII (Sly disease) | β-Glucuronidase |
| | MPS IX | Hylauronidase |
| Glycogen storage | Pompe (glycogen storage disease type II) | α-Glucosidase 2 |
| disease | | |
| Lipid metabolism | Lysosomal acid lipase deficiency (LAL-D; Wolman | Lysosomal acid lipase |
| | disease) | |

TABLE D-2
LSDs and corresponding Genes encoding Proteins
which can be fused to an anti-TfR for treatment

| | Mouse gene | Human gene | Human | OMIM | Genes per |
|---|---|---|---|---|---|
| OMIM disease name | entrez id | entrez id | gene | id | OMIM disease |
| Tangier disease | 11303 | 19 | ABCA1 | 205400 | 1 |
| Intellectual | 11305 | 20 | ABCA2 | 618808 | 1 |
| developmental | | | | | |
| disorder with poor | | | | | |
| growth and with or | | | | | |
| without seizures or | | | | | |
| ataxia | | | | | |
| Hypermethioninemia | 11534 | 132 | ADK | 614300 | 1 |
| due to adenosine | | | | | |
| kinase deficiency | | | | | |
| Aspartylglycosaminuria | | | AGA | | |
| Fructose intolerance, | 230163 | 229 | ALDOB | 229600 | 1 |
| hereditary | | | | | |
| MEDNIK syndrome | 11769 | 1174 | AP1S1 | 609313 | 1 |
| Spastic paraplegia 50, | 11781 | 9179 | AP4M1 | 612936 | 1 |
| autosomal recessive | | | | | |
| Sea-blue histiocyte | 11816 | 348 | APOE | 269600 | 1 |
| disease | | | | | |
| Adenine | 11821 | 353 | APRT | 614723 | 1 |
| phosphoribosyltransferase | | | | | |
| deficiency | | | | | |
| Maroteaux-Lamy | | | ARSB | | |
| syndrome | | | | | |
| (mucopolysaccharidosis | | | | | |
| type VI | | | | | |
| Mucopolysaccharidosis, | 77041 | 153642 | ARSK | 619698 | 1 |
| type X | | | | | |
| Spinocerebellar | 74244 | 10533 | ATG7 | 619422 | 1 |
| ataxia, autosomal | | | | | |
| recessive 31 | | | | | |
| Cutis laxa, autosomal | 11964 | 523 | ATP6V1A | 617403 | 1 |
| recessive, type IID | | | | | |
| Farber disease | | | ASAH1 | | |
| Hermansky-Pudlak | 18457 | 26258 | BLOC1S6 | 614171 | 1 |
| syndrome 9 | | | | | |
| Ceroid lipofuscinosis, | 76524 | 54982 | CLN6 | 601780 | 1 |
| neuronal, 6A | | | | | |
| Ceroid lipofuscinosis, | 76524 | 54982 | CLN6 | 204300 | 1 |
| neuronal, 6B (Kufs | | | | | |
| type) | | | | | |
| Ceroid lipofuscinosis, | 26889 | 2055 | CLN8 | 600143 | 1 |
| neuronal, 8 | | | | | |
| Ceroid lipofuscinosis, | 26889 | 2055 | CLN8 | 610003 | 1 |
| neuronal, 8, Northern | | | | | |
| epilepsy variant | | | | | |
| Galactosialidosis | 19025 | 5476 | CTSA | 256540 | 1 |
| cystinosis | | | CTNS | | |
| Haim-Munk | | | CTSC | | |
| syndrome, Papillon | | | | | |
| Lefevre Syndrome | | | | | |
| Ceroid lipofuscinosis, | 13033 | 1509 | CTSD | 610127 | 1 |
| neuronal, 10 | | | | | |
| Pycnodysostosis | 13038 | 1513 | CTSK | 265800 | 1 |
| Imerslund-Grasbeck | 65969 | 8029 | CUBN | 261100 | 1 |
| syndrome 1 | | | | | |
| Proteinuria, chronic | 65969 | 8029 | CUBN | 618884 | 1 |
| benign] | | | | | |
| WHIM syndrome 2 | 12765 | 3579 | CXCR2 | 619407 | 1 |
| Orthostatic | 13056 | 1534 | CYB561 | 618182 | 1 |
| hypotension 2 | | | | | |
| 5-fluorouracil toxicity | 99586 | 1806 | DPYD | 274270 | 1 |
| Dihydropyrimidine | 99586 | 1806 | DPYD | 274270 | 1 |
| dehydrogenase | | | | | |
| deficiency | | | | | |
| Cone-rod dystrophy | 67171 | 128338 | DRAM2 | 616502 | 1 |
| 21 | | | | | |
| Vici syndrome | 100502841 | 57724 | EPG5 | 242840 | 1 |
| Arthrogryposis | 67458 | 57222 | ERGIC1 | 208100 | 1 |
| multiplex congenita 2, | | | | | |
| neurogenic type | | | | | |
| Fucosidosis | 71665 | 2517 | FUCA1 | 230000 | 1 |
| Cataract 18, | 17281 | 79443 | FYCO1 | 610019 | 1 |
| autosomal recessive | | | | | |
| Pompe disease | | | GAA | | |
| Mucopolysaccharidosis | | | GALNS | | |
| IV | | | | | |
| Gaucher disease | | | GBA | | |
| Fabry disease | | | GLA | | |
| Mucolipidosis II | 432486 | 79158 | GNPTAB | 252500 | 1 |
| alpha/beta | | | | | |
| Mucolipidosis III | 432486 | 79158 | GNPTAB | 252600 | 1 |
| alpha/beta | | | | | |
| Mucolipidosis III | 214505 | 84572 | GNPTG | 252605 | 1 |
| gamma | | | | | |
| Mucopolysaccharidosis | | | GUSB | | |
| Type VII | | | | | |
| Tay Sachs Disease | | | HEXA | | |
| Sandhoff disease, | 15212 | 3074 | HEXB | 268800 | 1 |
| infantile, juvenile, and | | | | | |
| adult forms | | | | | |
| Mucopolysaccharidosis | 52120 | 138050 | HGSNAT | 252930 | 1 |
| type IIIC | | | | | |
| (Sanfilippo C) | | | | | |
| Retinitis pigmentosa | 52120 | 138050 | HGSNAT | 616544 | 1 |
| 73 | | | | | |
| Hermansky-Pudlak | 20170 | 79803 | HPS6 | 614075 | 1 |
| syndrome 6 | | | | | |
| Mucopolysaccharidosis | | | IDUA | | |
| I | | | | | |
| Mucopolysaccharidosis | | | IDS | | |
| II | | | | | |
| Spastic paraplegia, | 16594 | 64837 | KLC2 | 609541 | 1 |
| optic atrophy, and | | | | | |
| neuropathy | | | | | |
| Lysosomal acid lipase | | | LAL | | |
| defciency | | | | | |
| Danon disease | | | LAMP2 | | |
| Immunodeficiency 52 | 16797 | 27040 | LAT | 617514 | 1 |
| Leydig cell | 16867 | 3973 | LHCGR | 238320 | 1 |
| hypoplasia with | | | | | |
| hypergonadotropic | | | | | |
| hypogonadism | | | | | |
| Leydig cell | 16867 | 3973 | LHCGR | 238320 | 1 |
| hypoplasia with | | | | | |
| pseudohermaphroditism | | | | | |
| Luteinizing hormone | 16867 | 3973 | LHCGR | 238320 | 1 |
| resistance, female | | | | | |
| Immunodeficiency, | 80877 | 987 | LRBA | 614700 | 1 |
| common variable, 8, | | | | | |
| with autoimmunity | | | | | |
| Keratosis pilaris | 16971 | 4035 | LRP1 | 604093 | 1 |
| atrophicans | | | | | |
| Chediak-Higashi | 17101 | 1130 | LYST | 214500 | 1 |
| syndrome | | | | | |
| Alpha-Mannosidosis | | | MAN2B1 | | |
| Spondyloepiphyseal | | | MBTPS1 | | |
| Dysplasia, Kondo-Fu | | | | | |
| Type | | | | | |
| Mucolipidosis IV | | | MCOLN1 | | |
| Ceroid lipofuscinosis, | 72175 | 256471 | MFSD8 | 610951 | 1 |
| neuronal, 7 | | | | | |
| Macular dystrophy | 72175 | 256471 | MFSD8 | 616170 | 1 |
| with central cone | | | | | |
| involvement | | | | | |
| Megalencephalic | 170790 | 23209 | MLC1 | 604004 | 1 |
| leukoencephalopathy | | | | | |
| with subcortical cysts | | | | | |
| Myeloperoxidase | 17523 | 4353 | MPO | 254600 | 1 |
| deficiency | | | | | |
| Deafness, autosomal | 17921 | 4647 | MYO7A | 600060 | 1 |
| recessive 2 | | | | | |
| Usher syndrome, type | 17921 | 4647 | MYO7A | 276900 | 1 |
| 1B | | | | | |
| Kanzaki disease | 17939 | 4668 | NAGA | 609242 | 1 |
| Schindler disease, | 17939 | 4668 | NAGA | 609241 | 1 |
| type I | | | | | |
| Schindler disease, | 17939 | 4668 | NAGA | 609241 | 1 |
| type III | | | | | |
| Niemann-Pick | 18145 | 4864 | NPC1 | 257220 | 1 |
| disease, type C1 | | | | | |
| Niemann-Pick | 18145 | 4864 | NPC1 | 257220 | 1 |
| disease, type D | | | | | |
| Niemann-pick | 67963 | 10577 | NPC2 | 607625 | 1 |
| disease, type C2 | | | | | |
| Spastic paraplegia 45, | 76952 | 22978 | NT5C2 | 613162 | 1 |
| autosomal recessive | | | | | |
| Sialidosis | | | NEU1 | | |
| Parkinson disease 6, | 68943 | 65018 | PINK1 | 605909 | 1 |
| early onset | | | | | |
| Osteopetrosis, | 353047 | 9842 | PLEKHM1 | 611497 | 1 |
| autosomal recessive 6 | | | | | |
| Hemophagocytic | 18646 | 5551 | PRF1 | 603553 | 1 |
| lymphohistiocytosis, | | | | | |
| familial, 2 | | | | | |
| Epilepsy, progressive | 12492 | 950 | SCARB2 | 254900 | 1 |
| myoclonic 4, with or | | | | | |
| without renal failure | | | | | |
| Mucopolysaccharidos | 27029 | 6448 | SGSH | 252900 | 1 |
| is type IIIA | | | | | |
| (Sanfilippo A) | | | | | |
| Neurodevelopmental | 108037 | 6472 | SHMT2 | 619121 | 1 |
| disorder with | | | | | |
| cardiomyopathy, | | | | | |
| spasticity, and brain | | | | | |
| abnormalities | | | | | |
| Histiocytosis- | 71279 | 55315 | SLC29A3 | 602782 | 1 |
| lymphadenopathy | | | | | |
| plus syndrome | | | | | |
| Niemann-Pick | | | SMPD1 | | |
| disease, type A/B, | | | | | |
| acid | | | | | |
| sphingomyelinase | | | | | |
| deficiency | | | | | |
| Congenital disorder of | 67547 | 64116 | SLC39A8 | 616721 | 1 |
| glycosylation, type | | | | | |
| IIn | | | | | |
| Spinocerebellar | 244962 | 57231 | SNX14 | 616354 | 1 |
| ataxia, autosomal | | | | | |
| recessive 20 | | | | | |
| Amyotrophic lateral | 214585 | 80208 | SPG11 | 602099 | 1 |
| sclerosis 5, juvenile | | | | | |
| Charcot-Marie-Tooth | 214585 | 80208 | SPG11 | 616668 | 1 |
| disease, axonal, type | | | | | |
| 2X | | | | | |
| Spastic paraplegia 11, | 214585 | 80208 | SPG11 | 604360 | 1 |
| autosomal recessive | | | | | |
| Warburg micro | 67231 | 128637 | TBC1D20 | 615663 | 1 |
| syndrome 4 | | | | | |
| Dystonia 32 | 71732 | 55823 | VPS11 | 619637 | 1 |
| Leukodystrophy, | 71732 | 55823 | VPS11 | 616683 | 1 |
| hypomyelinating, 12 | | | | | |
| Choreoacanthocytosis | 271564 | 23230 | VPS13A | 200150 | 1 |
| Arthrogryposis, renal | 233405 | 26276 | VPS33B | 208085 | 1 |
| dysfunction, and | | | | | |
| cholestasis 1 | | | | | |
| Pontocerebellar | 68505 | 738 | VPS51 | 618606 | 1 |
| hypoplasia, type 13 | | | | | |
| Pontocerebellar | 68299 | 55275 | VPS53 | 615851 | 1 |
| hypoplasia, type 2E | | | | | |
| Neurodevelopmental | 66840 | 56270 | WDR45B | 617977 | 1 |
| disorder with spastic | | | | | |
| quadriplegia and brain | | | | | |
| abnormalities with or | | | | | |
| without seizures | | | | | |
| Cerebellar ataxia, | 192652 | 124997 | WDR81 | 610185 | 1 |
| mental retardation, | | | | | |
| and dysequilibrium | | | | | |
| syndrome 2 | | | | | |
| Hydrocephalus, | 192652 | 124997 | WDR81 | 617967 | 1 |
| congenital, 3, with | | | | | |
| brain anomalies | | | | | |
| Xanthinuria, type I | 22436 | 7498 | XDH | 278300 | 1 |
| Spastic paraplegia 15, | 211978 | 23503 | ZFYVE26 | 270700 | 1 |
| autosomal recessive | | | | | |

Thus, provided herein are anti-TfR:LSD protein fusions wherein the LSD fusion protein is as set forth in Table D-1 and D-2 as well as methods for treating or preventing the corresponding LSD in Table D-1 and D-2 in a patient by administering an effective amount of anti-TfR:LSD protein fusion to the patient.

Options for the treatment of lysosomal storage diseases include enzyme replacement therapy (ERT), substrate reduction therapy, pharmacological chaperone-mediated therapy, hematopoietic stem cell transplant therapy, and gene therapy. An example of substrate reduction therapy is Miglustat or Eliglustat for treating Gaucher Type 1. These drugs act by blocking synthase activity, which reduces subsequent substrate production. Hematopoietic stem cell therapy (HSCT), for example, is used to ameliorate and slow-down the negative central nervous system phenotype in patients with some forms of MPS. See R. M. Boustany, “Lysosomal storage diseases—the horizon expands,” 9(10) Nat. Rev. Neurol. 583-98, October 2013; which reference is incorporated herein in its entirety by reference. Thus, provided herein are anti-TfR:Payload fusion proteins wherein the payload is an enzyme replacement therapy (ERT) agent, substrate reduction therapy agent (e.g., Miglustat), pharmacological chaperone-mediated therapy agent, or gene therapy agent as well as methods for treating LSDs in a patient by administering an effective amount of such a fusion protein to the patient.

Two LSDs are Pompe disease and Fabry disease. As discussed herein, Pompe disease is caused by defective lysosomal enzyme alpha-glucosidase (GAA), which results in the deficient processing of lysosomal glycogen. Thus, Pompe disease may also be referred to as a glycogen storage disease. Thus, provided herein are anti-TfR:Payload fusion proteins wherein the payload is GAA as well as methods for treating Pompe disease in a patient by administering an effective amount of such a fusion protein to the patient.

Fabry disease is caused by defective lysosomal enzyme alpha-galactosidase A (GLA), which results in the accumulation of globotriaosylceramide within the blood vessels and other tissues and organs. Symptoms associated with Fabry disease include pain from nerve damage and/or small vascular obstruction, renal insufficiency and eventual failure, cardiac complications such as high blood pressure and cardiomyopathy, dermatological symptoms such as formation of angiokeratomas, anhidrosis or hyperhidrosis, and ocular problems such as cornea verticillata, spoke-like cataract, and conjunctival and retinal vascular abnormalities. Treatments include FABRAZYIE (agalsidase beta), REPLAGAL (agalsidase alfa) and GALAFOLD. Thus, provided herein are anti-TfR:Payload fusion proteins wherein the payload is alpha-galactosidase A, agalsidase beta, agalsidase alfa or miglastat as well as methods for treating Fabry disease in a patient by administering an effective amount of such a fusion protein to the patient.

“Alpha-galactosidase A” (GLA or “α-galactosidase A”) facilitates the hydrolysis of terminal α-galactosyl moieties from glycolipids and glycoproteins, and also hydrolyses α-D-fucosides. GLA is also known inter alia as EC 3.2.1.22, melibiase, α-D-galactosidase, α-galactosidase A, α-galactoside galactohydrolase, α-D-galactoside galactohydrolase.

### VI. Heart Diseases and Disorders

Provided herein are anti-TfR:Payload fusion proteins e.g., wherein the antigen-binding protein of the fusion (e.g., scFv) is 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263, and wherein the payload is a heart disease or disorder therapeutic agent, e.g., a protein that is set forth below in Table E or a variant thereof. Methods for treating or preventing a heart disease or disorder that is listed below in Table E, in a patient in need thereof, by administering an effective amount of an anti-TfR:Payload to the patient wherein the payload is a protein encoded by a gene corresponding to the particular heart disease or disorder in Table E.

TABLE E
Heart Diseases and Disorders and corresponding Genes encoding
Proteins which can be fused to an anti-TfR for treatment.

| | Mouse gene | Human gene | Human | OMIM | Genes per |
|---|---|---|---|---|---|
| OMIM disease name | entrez id | entrez id | gene | id | OMIM disease |
| Intellectual disability and | 20928 | 10060 | ABCC9 | 619719 | 1 |
| myopathy syndrome | | | | | |
| Muscular dystrophy, limb-girdle, | 23828 | 11149 | BVES | 616812 | 1 |
| autosomal recessive 25 | | | | | |
| Neurodevelopmental disorder with | 12287 | 774 | CACNA1B | 618497 | 1 |
| seizures and nonepileptic | | | | | |
| hyperkinetic movements | | | | | |
| Cerebellar atrophy with seizures | 56808 | 9254 | CACNA2D2 | 618501 | 1 |
| and variable developmental delay | | | | | |
| Ventricular tachycardia, | 12373 | 845 | CASQ2 | 611938 | 1 |
| catecholaminergic polymorphic, 2 | | | | | |
| Lipodystrophy, congenital | 12389 | 857 | CAV1 | 612526 | 1 |
| generalized, type 3 | | | | | |
| Arrhythmogenic right ventricular | 13506 | 1824 | DSC2 | 610476 | 1 |
| dysplasia 11 | | | | | |
| Arrhythmogenic right ventricular | 13506 | 1824 | DSC2 | 610476 | 1 |
| dysplasia 11 with mild | | | | | |
| palmoplantar keratoderma and | | | | | |
| woolly hair | | | | | |
| Cardiomyopathy, dilated, with | 109620 | 1832 | DSP | 605676 | 1 |
| woolly hair and keratoderma | | | | | |
| Epidermolysis bullosa, lethal | 109620 | 1832 | DSP | 609638 | 1 |
| acantholytic | | | | | |
| Skin fragility-woolly hair | 109620 | 1832 | DSP | 607655 | 1 |
| syndrome | | | | | |
| Congenital heart defects, multiple | 14464 | 140628 | GATA5 | 617912 | 1 |
| types, 5 | | | | | |
| Hemolytic anemia due to | 14775 | 2876 | GPX1 | 614164 | 1 |
| glutathione peroxidase deficiency | | | | | |
| Naxos disease | 16480 | 3728 | JUP | 601214 | 1 |
| Jervell and Lange-Nielsen | 16509 | 3753 | KCNE1 | 612347 | 1 |
| syndrome 2 | | | | | |
| Myopathy, myofibrillar, 12, | 17906 | 4633 | MYL2 | 619424 | 1 |
| infantile-onset, with | | | | | |
| cardiomyopathy | | | | | |
| Cardiomyopathy, hypertrophic, 8 | 17897 | 4634 | MYL3 | 608751 | 1 |
| Nephrotic syndrome, type 22 | 70729 | 9722 | NOS1AP | 619155 | 1 |
| Developmental and epileptic | 20266 | 6324 | SCN1B | 617350 | 1 |
| encephalopathy 52 | | | | | |
| Dicarboxylic aminoaciduria | 20510 | 6505 | SLC1A1 | 222730 | 1 |
| Lichtenstein-Knorr syndrome | 20544 | 6548 | SLC9A1 | 616291 | 1 |
| Hypogonadotropic hypogonadism | 21338 | 6870 | TACR3 | 614840 | 1 |
| 11 with or without anosmia | | | | | |
| Segawa syndrome, recessive | 21823 | 7054 | TH | 605407 | 1 |

### VII. Central Nervous System (CNS) Diseases and Disorders

Provided herein are anti-TfR:Payload fusion proteins e.g., wherein the antigen-binding protein of the fusion (e.g., scFv) is 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263, and wherein the payload is a CNS disease or disorder therapeutic agent, e.g., a protein that is set forth below in Table F or a variant thereof. Methods for treating or preventing a CNS disease or disorder that is listed below in Table F, in a patient in need thereof, by administering an effective amount of an anti-TfR:Payload to the patient wherein the payload is a protein encoded by a gene corresponding to the particular CNS disease or disorder in Table F.

TABLE F
CNS Disease or Disorder and Corresponding Genes Encoding
Proteins which Can Be Fused to an Anti-TfR for Treatment.

| | Mouse gene | Human gene | Human | OMIM | Genes per |
|---|---|---|---|---|---|
| OMIM disease name | entrez id | entrez id | gene | id | OMIM disease |
| Intellectual developmental disorder | 11305 | 20 | ABCA2 | 618808 | 1 |
| with poor growth and with or | | | | | |
| without seizures or ataxia | | | | | |
| Spondyloepimetaphyseal dysplasia, | 11595 | 176 | ACAN | 612813 | 1 |
| aggrecan type | | | | | |
| Neurodevelopmental disorder with | 110532 | 104 | ADARB1 | 618862 | 1 |
| hypotonia, microcephaly, and | | | | | |
| seizures | | | | | |
| Microcephaly 16, primary, | 71782 | 23141 | ANKLE2 | 616681 | 1 |
| autosomal recessive | | | | | |
| Spinocerebellar ataxia, autosomal | 74244 | 10533 | ATG7 | 619422 | 1 |
| recessive 31 | | | | | |
| Acromesomelic dysplasia 3 | 12167 | 658 | BMPR1B | 609441 | 1 |
| Elsahy-Waters syndrome | 12552 | 1009 | CDH11 | 211380 | 1 |
| Ceroid lipofuscinosis, neuronal, 8 | 26889 | 2055 | CLN8 | 600143 | 1 |
| Ceroid lipofuscinosis, neuronal, 8, | 26889 | 2055 | CLN8 | 610003 | 1 |
| Northern epilepsy variant | | | | | |
| Pitt-Hopkins like syndrome 1 | 66797 | 26047 | CNTNAP2 | 610042 | 1 |
| Gaze palsy, familial horizontal, with | 13176 | 1630 | DCC | 617542 | 1 |
| progressive scoliosis, 2 | | | | | |
| Short-rib thoracic dysplasia 3 with | 110350 | 79659 | DYNC2H1 | 613091 | 1 |
| or without polydactyly | | | | | |
| Bleeding disorder, platelet-type, 22 | 13844 | 2048 | EPHB2 | 618462 | 1 |
| Macrocephaly, dysmorphic facies, | 235439 | 8925 | HERC1 | 617011 | 1 |
| and psychomotor retardation | | | | | |
| Charcot-Marie-Tooth disease, | 20589 | 3508 | IGHMBP2 | 616155 | 1 |
| axonal, type 2S | | | | | |
| Neuronopathy, distal hereditary | 20589 | 3508 | IGHMBP2 | 604320 | 1 |
| motor, type VI | | | | | |
| SESAME syndrome | 16513 | 3766 | KCNJ10 | 612780 | 1 |
| Goldberg-Shprintzen megacolon | 72320 | 26128 | KIFBP | 609460 | 1 |
| syndrome | | | | | |
| Obesity, morbid, due to leptin | 16846 | 3952 | LEP | 614962 | 1 |
| deficiency | | | | | |
| Spastic paraplegia 75, autosomal | 17136 | 4099 | MAG | 616680 | 1 |
| recessive | | | | | |
| Hypogonadotropic hypogonadism | 18072 | 4808 | NHLH2 | 619755 | 1 |
| 27 without anosmia | | | | | |
| Seckel syndrome 7 | 18080 | 51199 | NIN | 614851 | 1 |
| Pitt-Hopkins-like syndrome 2 | 18189 | 9378 | NRXN1 | 614325 | 1 |
| Oxoglutarate dehydrogenase | 18293 | 4967 | OGDH | 203740 | 1 |
| deficiency | | | | | |
| Myopathy, congenital, progressive, | 18509 | 5081 | PAX7 | 618578 | 1 |
| with scoliosis | | | | | |
| Epilepsy, progressive myoclonic, 10 | 77630 | 56978 | PRDM8 | 616640 | 1 |
| Lissencephaly 2 (Norman-Roberts | 19699 | 5649 | RELN | 257320 | 1 |
| type) | | | | | |
| Thyroid hormone metabolism, | 75420 | 79048 | SECISBP2 | 609698 | 1 |
| abnormal | | | | | |
| Thyroid hormone metabolism, | 75420 | 79048 | SECISBP2 | 609698 | 1 |
| abnormal, 1 | | | | | |
| Neuropathy, hereditary motor and | 67453 | 91137 | SLC25A46 | 616505 | 1 |
| sensory, type VIB | | | | | |
| Pontocerebellar hypoplasia, type 1E | 67453 | 91137 | SLC25A46 | 619303 | 1 |
| Amyotrophic lateral sclerosis 5, | 214585 | 80208 | SPG11 | 602099 | 1 |
| juvenile | | | | | |
| Charcot-Marie-Tooth disease, | 214585 | 80208 | SPG11 | 616668 | 1 |
| axonal, type 2X | | | | | |
| Spastic paraplegia 11, autosomal | 214585 | 80208 | SPG11 | 604360 | 1 |
| recessive | | | | | |
| Netherton syndrome | 72432 | 11005 | SPINK5 | 256500 | 1 |
| Joubert syndrome 13 | 654470 | 79600 | TCTN1 | 614173 | 1 |
| Microphthalmia, syndromic 11 | 22326 | 11023 | VAX1 | 614402 | 1 |
| Osteogenesis imperfecta, type XV | 22408 | 7471 | WNT1 | 615220 | 1 |

### VIII. Eye Diseases and Disorders

Provided herein are anti-TfR:Payload fusion proteins e.g., wherein the antigen-binding protein of the fusion (e.g., scFv) is 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263, and wherein the payload is an eye disease or disorder therapeutic agent, e.g., a protein that is set forth below in Table G or a variant thereof. Methods for treating or preventing an eye disease or disorder that is listed below in Table G, in a patient in need thereof, by administering an effective amount of an anti-TfR:Payload to the patient wherein the payload is a protein encoded by a gene corresponding to the particular eye disease or disorder in Table G.

TABLE G
Eye Disease or Disorder and Corresponding Genes Encoding
Proteins which Can Be Fused to an Anti-TfR for Treatment.

| | Mouse gene | Human gene | Human | OMIM | Genes per |
|---|---|---|---|---|---|
| OMIM disease name | entrez id | entrez id | gene | id | OMIM disease |
| Intellectual developmental disorder | 11305 | 20 | ABCA2 | 618808 | 1 |
| with poor growth and with or | | | | | |
| without seizures or ataxia | | | | | |
| Microcornea, myopic chorioretinal | 208936 | 170692 | ADAMTS18 | 615458 | 1 |
| atrophy, and telecanthus | | | | | |
| Microphthalmia, isolated 8 | 56847 | 220 | ALDH1A3 | 615113 | 1 |
| Fructose intolerance, hereditary | 230163 | 229 | ALDOB | 229600 | 1 |
| Alstrom syndrome | 236266 | 7840 | ALMS1 | 203800 | 1 |
| Sea-blue histiocyte disease | 11816 | 348 | APOE | 269600 | 1 |
| Mucopolysaccharidosis, type X | 77041 | 153642 | ARSK | 619698 | 1 |
| Cutis laxa, autosomal recessive, | 11964 | 523 | ATP6V1A | 617403 | 1 |
| type IID | | | | | |
| Bardet-Biedl syndrome 4 | 102774 | 585 | BBS4 | 615982 | 1 |
| Bardet-Biedl syndrome 7 | 71492 | 55212 | BBS7 | 615984 | 1 |
| Acromesomelic dysplasia 3 | 12167 | 658 | BMPR1B | 609441 | 1 |
| Cone-rod synaptic disorder, | 73660 | 57010 | CABP4 | 610427 | 1 |
| congenital nonprogressive | | | | | |
| Joubert syndrome 5 | 216274 | 80184 | CEP290 | 610188 | 1 |
| Leber congenital amaurosis 10 | 216274 | 80184 | CEP290 | 611755 | 1 |
| Meckel syndrome 4 | 216274 | 80184 | CEP290 | 611134 | 1 |
| Senior-Loken syndrome 6 | 216274 | 80184 | CEP290 | 610189 | 1 |
| Complement factor D deficiency | 11537 | 1675 | CFD | 613912 | 1 |
| Ceroid lipofuscinosis, neuronal, 8 | 26889 | 2055 | CLN8 | 600143 | 1 |
| Ceroid lipofuscinosis, neuronal, 8, | 26889 | 2055 | CLN8 | 610003 | 1 |
| Northern epilepsy variant | | | | | |
| Achromatopsia 2 | 12790 | 1261 | CNGA3 | 216900 | 1 |
| Focal segmental | 241324 | 286204 | CRB2 | 616220 | 1 |
| glomerulosclerosis 9 | | | | | |
| Ventriculomegaly with cystic | 241324 | 286204 | CRB2 | 219730 | 1 |
| kidney disease | | | | | |
| Leber congenital amaurosis 7 | 12951 | 1406 | CRX | 613829 | 1 |
| Cataract 22 | 12962 | 1417 | CRYBB3 | 609741 | 1 |
| Galactosialidosis | 19025 | 5476 | CTSA | 256540 | 1 |
| Ceroid lipofuscinosis, neuronal, 10 | 13033 | 1509 | CTSD | 610127 | 1 |
| Pycnodysostosis | 13038 | 1513 | CTSK | 265800 | 1 |
| Imerslund-Grasbeck syndrome 1 | 65969 | 8029 | CUBN | 261100 | 1 |
| Proteinuria, chronic benign] | 65969 | 8029 | CUBN | 618884 | 1 |
| WHIM syndrome 2 | 12765 | 3579 | CXCR2 | 619407 | 1 |
| Cone-rod dystrophy 21 | 67171 | 128338 | DRAM2 | 616502 | 1 |
| Vici syndrome | 100502841 | 57724 | EPG5 | 242840 | 1 |
| Bleeding disorder, platelet-type, 22 | 13844 | 2048 | EPHB2 | 618462 | 1 |
| Anterior segment dysgenesis 2, | 30923 | 2301 | FOXE3 | 610256 | 1 |
| multiple subtypes | | | | | |
| Fucosidosis | 71665 | 2517 | FUCA1 | 230000 | 1 |
| Cataract 18, autosomal recessive | 17281 | 79443 | FYCO1 | 610019 | 1 |
| Ectodermal dysplasia/short stature | 252973 | 79977 | GRHL2 | 616029 | 1 |
| syndrome | | | | | |
| Night blindness, congenital | 108072 | 2916 | GRM6 | 257270 | 1 |
| stationary (complete), 1B, | | | | | |
| autosomal recessive | | | | | |
| Growth hormone deficiency with | 15209 | 8820 | HESX1 | 182230 | 1 |
| pituitary anomalies | | | | | |
| Pituitary hormone deficiency, | 15209 | 8820 | HESX1 | 182230 | 1 |
| combined, 5 | | | | | |
| Septooptic dysplasia | 15209 | 8820 | HESX1 | 182230 | 1 |
| Sandhoff disease, infantile, | 15212 | 3074 | HEXB | 268800 | 1 |
| juvenile, and adult forms | | | | | |
| Mucopolysaccharidosis type IIIC | 52120 | 138050 | HGSNAT | 252930 | 1 |
| (Sanfilippo C) | | | | | |
| Retinitis pigmentosa 73 | 52120 | 138050 | HGSNAT | 616544 | 1 |
| Hermansky-Pudlak syndrome 6 | 20170 | 79803 | HPS6 | 614075 | 1 |
| Cerebellar atrophy, developmental | 16531 | 3778 | KCNMA1 | 617643 | 1 |
| delay, and seizures | | | | | |
| Cornea plana 2, autosomal | 16545 | 11081 | KERA | 217300 | 1 |
| recessive | | | | | |
| Spastic paraplegia, optic atrophy, | 16594 | 64837 | KLC2 | 609541 | 1 |
| and neuropathy | | | | | |
| Poretti-Boltshauser syndrome | 16772 | 284217 | LAMA1 | 615960 | 1 |
| Cortical malformations, occipital | 23928 | 10319 | LAMC3 | 614115 | 1 |
| Leydig cell hypoplasia with | 16867 | 3973 | LHCGR | 238320 | 1 |
| hypergonadotropic hypogonadism | | | | | |
| Leydig cell hypoplasia with | 16867 | 3973 | LHCGR | 238320 | 1 |
| pseudohermaphroditism | | | | | |
| Luteinizing hormone resistance, | 16867 | 3973 | LHCGR | 238320 | 1 |
| female | | | | | |
| Immunodeficiency, common | 80877 | 987 | LRBA | 614700 | 1 |
| variable, 8, with autoimmunity | | | | | |
| Microphthalmia/coloboma and | 23937 | 10586 | MAB21L2 | 615877 | 1 |
| skeletal dysplasia syndrome | | | | | |
| Charcot-Marie-Tooth disease, | 170731 | 9927 | MFN2 | 617087 | 1 |
| axonal, type 2A2B | | | | | |
| Neurodevelopmental disorder with | 76574 | 84879 | MFSD2A | 616486 | 1 |
| progressive microcephaly, | | | | | |
| spasticity, and brain abnormalities | | | | | |
| Ceroid lipofuscinosis, neuronal, 7 | 72175 | 256471 | MFSD8 | 610951 | 1 |
| Macular dystrophy with central | 72175 | 256471 | MFSD8 | 616170 | 1 |
| cone involvement | | | | | |
| Megalencephalic | 170790 | 23209 | MLC1 | 604004 | 1 |
| leukoencephalopathy with | | | | | |
| subcortical cysts | | | | | |
| Myeloperoxidase deficiency | 17523 | 4353 | MPO | 254600 | 1 |
| Kanzaki disease | 17939 | 4668 | NAGA | 609242 | 1 |
| Schindler disease, type I | 17939 | 4668 | NAGA | 609241 | 1 |
| Schindler disease, type III | 17939 | 4668 | NAGA | 609241 | 1 |
| Intellectual developmental disorder | 18145 | 4864 | NPC1 | 257220 | 1 |
| with poor growth and with or | | | | | |
| without seizures or ataxia | | | | | |
| Microcornea, myopic chorioretinal | 18145 | 4864 | NPC1 | 257220 | 1 |
| atrophy, and telecanthus | | | | | |
| Microphthalmia, isolated 8 | 67963 | 10577 | NPC2 | 607625 | 1 |
| Fructose intolerance, hereditary | 53885 | 4867 | NPHP1 | 609583 | 1 |
| Alstrom syndrome | 53885 | 4867 | NPHP1 | 256100 | 1 |
| Sea-blue histiocyte disease | 53885 | 4867 | NPHP1 | 266900 | 1 |
| Mucopolysaccharidosis, type X | 18541 | 5116 | PCNT | 210720 | 1 |
| Cutis laxa, autosomal recessive, | 225600 | 5145 | PDE6A | 613810 | 1 |
| type IID | | | | | |
| Bardet-Biedl syndrome 4 | 18587 | 5158 | PDE6B | 613801 | 1 |
| Bardet-Biedl syndrome 7 | 18742 | 5309 | PITX3 | 610623 | 1 |
| Acromesomelic dysplasia 3 | 18742 | 5309 | PITX3 | 610623 | 1 |
| Cone-rod synaptic disorder, | 353047 | 9842 | PLEKHM1 | 611497 | 1 |
| congenital nonprogressive | | | | | |
| Joubert syndrome 5 | 18646 | 5551 | PRF1 | 603553 | 1 |
| Leber congenital amaurosis 10 | 69453 | 646960 | PRSS56 | 613517 | 1 |
| Meckel syndrome 4 | 69675 | 7837 | PXDN | 269400 | 1 |
| Senior-Loken syndrome 6 | 226407 | 22930 | RAB3GAP1 | 619420 | 1 |
| Complement factor D deficiency | 226407 | 22930 | RAB3GAP1 | 600118 | 1 |
| Ceroid lipofuscinosis, neuronal, 8 | 19662 | 5950 | RBP4 | 615147 | 1 |
| Ceroid lipofuscinosis, neuronal, 8, | 74023 | 343035 | RD3 | 610612 | 1 |
| Northern epilepsy variant | | | | | |
| Achromatopsia 2 | 244585 | 23322 | RPGRIP1L | 619113 | 1 |
| Focal segmental | 244585 | 23322 | RPGRIP1L | 611560 | 1 |
| glomerulosclerosis 9 | | | | | |
| Ventriculomegaly with cystic | 244585 | 23322 | RPGRIP1L | 611561 | 1 |
| kidney disease | | | | | |
| Leber congenital amaurosis 7 | 244891 | 49855 | SCAPER | 618195 | 1 |
| Cataract 22 | 12492 | 950 | SCARB2 | 254900 | 1 |
| Galactosialidosis | 27029 | 6448 | SGSH | 252900 | 1 |
| Ceroid lipofuscinosis, neuronal, 10 | 20510 | 6505 | SLC1A1 | 222730 | 1 |
| Pycnodysostosis | 71279 | 55315 | SLC29A3 | 602782 | 1 |
| Imerslund-Grasbeck syndrome 1 | 67547 | 64116 | SLC39A8 | 616721 | 1 |
| Proteinuria, chronic benign] | 71997 | 56006 | SMG9 | 616920 | 1 |
| WHIM syndrome 2 | 64075 | 64093 | SMOC1 | 206920 | 1 |
| Cone-rod dystrophy 21 | 244962 | 57231 | SNX14 | 616354 | 1 |
| Vici syndrome | 216892 | 124976 | SPNS2 | 618457 | 1 |
| Bleeding disorder, platelet-type, 22 | 67231 | 128637 | TBC1D20 | 615663 | 1 |
| Anterior segment dysgenesis 2, | 21823 | 7054 | TH | 605407 | 1 |
| multiple subtypes | | | | | |
| Fucosidosis | 17364 | 4308 | TRPM1 | 613216 | 1 |
| Cataract 18, autosomal recessive | 13345 | 117581 | TWIST2 | 227260 | 1 |
| Ectodermal dysplasia/short stature | 72088 | 10083 | USH1C | 602092 | 1 |
| syndrome | | | | | |
| Night blindness, congenital | 72088 | 10083 | USH1C | 276904 | 1 |
| stationary (complete), 1B, | | | | | |
| autosomal recessive | | | | | |
| Growth hormone deficiency with | 22326 | 11023 | VAX1 | 614402 | 1 |
| pituitary anomalies | | | | | |
| Pituitary hormone deficiency, | 71732 | 55823 | VPS11 | 619637 | 1 |
| combined, 5 | | | | | |
| Septooptic dysplasia | 71732 | 55823 | VPS11 | 616683 | 1 |
| Sandhoff disease, infantile, | 271564 | 23230 | VPS13A | 200150 | 1 |
| juvenile, and adult forms | | | | | |
| Mucopolysaccharidosis type IIIC | 233405 | 26276 | VPS33B | 208085 | 1 |
| (Sanfilippo C) | | | | | |
| Retinitis pigmentosa 73 | 66840 | 56270 | WDR45B | 617977 | 1 |
| Hermansky-Pudlak syndrome 6 | 192652 | 124997 | WDR81 | 610185 | 1 |
| Cerebellar atrophy, developmental | 192652 | 124997 | WDR81 | 617967 | 1 |
| delay, and seizures | | | | | |
| Cornea plana 2, autosomal | 211978 | 23503 | ZFYVE26 | 270700 | 1 |
| recessive | | | | | |

### IX. Brain Diseases and Disorders

Provided herein are anti-TfR:Payload fusion proteins e.g., wherein the antigen-binding protein of the fusion (e.g., scFv) is 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263, and wherein the payload is a brain disease or disorder therapeutic agent, e.g., a protein that is set forth below in Table H or a variant thereof. Methods for treating or preventing a brain disease or disorder that is listed below in Table H, in a patient in need thereof, by administering an effective amount of an anti-TfR:Payload to the patient wherein the payload is a protein encoded by a gene corresponding to the particular brain disease or disorder in Table H.

TABLE H
Brain Disease or Disorder and Corresponding Genes Encoding
Proteins which Can Be Fused to an Anti-TfR for Treatment.

| | Mouse gene | Human gene | Human | OMIM | Genes per |
|---|---|---|---|---|---|
| OMIM disease name | entrez id | entrez id | gene | id | OMIM disease |
| Deafness, autosomal recessive 44 | 432530 | 107 | ADCY1 | 610154 | 1 |
| Microcephaly 5, primary, | 12316 | 259266 | ASPM | 608716 | 1 |
| autosomal recessive | | | | | |
| Spinocerebellar ataxia, autosomal | 74244 | 10533 | ATG7 | 619422 | 1 |
| recessive 31 | | | | | |
| Bardet-Biedl syndrome 2 | 67378 | 583 | BBS2 | 615981 | 1 |
| Retinitis pigmentosa 74 | 67378 | 583 | BBS2 | 616562 | 1 |
| Bardet-Biedl syndrome 4 | 102774 | 585 | BBS4 | 615982 | 1 |
| Pitt-Hopkins like syndrome 1 | 66797 | 26047 | CNTNAP2 | 610042 | 1 |
| Joubert syndrome 17 | 73692 | 65250 | CPLANE1 | 614615 | 1 |
| Orofaciodigital syndrome VI | 73692 | 65250 | CPLANE1 | 277170 | 1 |
| Oculocutaneous albinism, type | 13190 | 1638 | DCT | 619165 | 1 |
| VIII | | | | | |
| Hermansky-Pudlak syndrome 7 | 94245 | 84062 | DTNBP1 | 614076 | 1 |
| Short-rib thoracic dysplasia 3 with | 110350 | 79659 | DYNC2H1 | 613091 | 1 |
| or without polydactyly | | | | | |
| Macrocephaly, dysmorphic facies, | 235439 | 8925 | HERC1 | 617011 | 1 |
| and psychomotor retardation | | | | | |
| Growth hormone deficiency with | 15209 | 8820 | HESX1 | 182230 | 1 |
| pituitary anomalies | | | | | |
| Pituitary hormone deficiency, | 15209 | 8820 | HESX1 | 182230 | 1 |
| combined, 5 | | | | | |
| Septooptic dysplasia | 15209 | 8820 | HESX1 | 182230 | 1 |
| Intellectual developmental | 77582 | 79143 | MBOAT7 | 617188 | 1 |
| disorder, autosomal recessive 57 | | | | | |
| Neurodevelopmental disorder with | 76574 | 84879 | MFSD2A | 616486 | 1 |
| progressive microcephaly, | | | | | |
| spasticity, and brain abnormalities | | | | | |
| Hypogonadotropic hypogonadism | 18072 | 4808 | NHLH2 | 619755 | 1 |
| 27 without anosmia | | | | | |
| Pitt-Hopkins-like syndrome 2 | 18189 | 9378 | NRXN1 | 614325 | 1 |
| Oxoglutarate dehydrogenase | 18293 | 4967 | OGDH | 203740 | 1 |
| deficiency | | | | | |
| Microcephalic osteodysplastic | 18541 | 5116 | PCNT | 210720 | 1 |
| primordial dwarfism, type II | | | | | |
| Intellectual developmental disorder | 207728 | 5138 | PDE2A | 619150 | 1 |
| with paroxysmal dyskinesia or | | | | | |
| seizures | | | | | |
| Neurodevelopmental disorder with | 241062 | 80055 | PGAP1 | 615802 | 1 |
| dysmorphic features, spasticity, | | | | | |
| and brain abnormalities | | | | | |
| Developmental and epileptic | 18795 | 23236 | PLCB1 | 613722 | 1 |
| encephalopathy 12 | | | | | |
| Martsolf syndrome 2 | 226407 | 22930 | RAB3GAP1 | 619420 | 1 |
| Warburg micro syndrome 1 | 226407 | 22930 | RAB3GAP1 | 600118 | 1 |
| Lissencephaly 2 (Norman-Roberts | 19699 | 5649 | RELN | 257320 | 1 |
| type) | | | | | |
| COACH syndrome 3 | 244585 | 23322 | RPGRIP1L | 619113 | 1 |
| Joubert syndrome 7 | 244585 | 23322 | RPGRIP1L | 611560 | 1 |
| Meckel syndrome 5 | 244585 | 23322 | RPGRIP1L | 611561 | 1 |
| Thyroid hormone metabolism, | 75420 | 79048 | SECISBP2 | 609698 | 1 |
| abnormal | | | | | |
| Thyroid hormone metabolism, | 75420 | 79048 | SECISBP2 | 609698 | 1 |
| abnormal, 1 | | | | | |
| Neuropathy, hereditary motor and | 67453 | 91137 | SLC25A46 | 616505 | 1 |
| sensory, type VIB | | | | | |
| Pontocerebellar hypoplasia, type | 67453 | 91137 | SLC25A46 | 619303 | 1 |
| 1E | | | | | |
| Spinocerebellar ataxia, autosomal | 20743 | 6712 | SPTBN2 | 615386 | 1 |
| recessive 14 | | | | | |
| Microcephaly-capillary | 70527 | 10617 | STAMBP | 614261 | 1 |
| malformation syndrome | | | | | |
| Neurodevelopmental disorder, | 21960 | 7143 | TNR | 619653 | 1 |
| nonprogressive, with spasticity and | | | | | |
| transient opisthotonus | | | | | |
| Intellectual developmental | 76510 | 83696 | TRAPPC9 | 613192 | 1 |
| disorder, autosomal recessive 13 | | | | | |
| Microcephaly 2, primary, | 233064 | 284403 | WDR62 | 604317 | 1 |
| autosomal recessive, with or | | | | | |
| without cortical malformations | | | | | |
| Osteogenesis imperfecta, type XV | 22408 | 7471 | WNT1 | 615220 | 1 |
| Diarrhea 9 | 22414 | 7482 | WNT2B | 618168 | 1 |

### X. Spinal Cord Diseases and Disorders

Provided herein are anti-TfR:Payload fusion proteins e.g., wherein the antigen-binding protein of the fusion (e.g., scFv) is 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263, and wherein the payload is a spinal cord disease or disorder therapeutic agent, e.g., a protein that is set forth below in Table I or a variant thereof. Methods for treating or preventing a spinal cord disease or disorder that is listed below in Table I, in a patient in need thereof, by administering an effective amount of an anti-TfR:Payload to the patient wherein the payload is a protein encoded by a gene corresponding to the particular spinal cord disease or disorder in Table I.

TABLE I
Spinal Cord Disease or Disorder and Corresponding Genes Encoding
Proteins which Can Be Fused to an Anti-TfR for Treatment.

| | Mouse gene | Human gene | Human | OMIM | Genes per |
|---|---|---|---|---|---|
| OMIM disease name | entrez id | entrez id | gene | id | OMIM disease |
| Neurodevelopmental disorder with | 110532 | 104 | ADARB1 | 618862 | 1 |
| hypotonia, microcephaly, and | | | | | |
| seizures | | | | | |
| Ceroid lipofuscinosis, neuronal, 8 | 26889 | 2055 | CLN8 | 600143 | 1 |
| Ceroid lipofuscinosis, neuronal, 8, | 26889 | 2055 | CLN8 | 610003 | 1 |
| Northern epilepsy variant | | | | | |
| Nephrotic syndrome, type 24 | 76441 | 23500 | DAAM2 | 619263 | 1 |
| Gaze palsy, familial horizontal, | 13176 | 1630 | DCC | 617542 | 1 |
| with progressive scoliosis, 2 | | | | | |
| Short-rib thoracic dysplasia 3 with | 110350 | 79659 | DYNC2H1 | 613091 | 1 |
| or without polydactyly | | | | | |
| Charcot-Marie-Tooth disease, | 20589 | 3508 | IGHMBP2 | 616155 | 1 |
| axonal, type 2S | | | | | |
| Neuronopathy, distal hereditary | 20589 | 3508 | IGHMBP2 | 604320 | 1 |
| motor, type VI | | | | | |
| Myopathy, congenital, progressive, | 18509 | 5081 | PAX7 | 618578 | 1 |
| with scoliosis | | | | | |
| Neu-Laxova syndrome 1 | 236539 | 26227 | PHGDH | 256520 | 1 |
| Phosphoglycerate dehydrogenase | 236539 | 26227 | PHGDH | 601815 | 1 |
| deficiency | | | | | |
| Carpenter syndrome | 19335 | 51715 | RAB23 | 201000 | 1 |
| Lissencephaly 2 (Norman-Roberts | 19699 | 5649 | RELN | 257320 | 1 |
| type) | | | | | |
| Joubert syndrome 13 | 654470 | 79600 | TCTN1 | 614173 | 1 |
| Cerebellar hypoplasia and mental | 22359 | 7436 | VLDLR | 224050 | 1 |
| retardation with or without | | | | | |
| quadrupedal locomotion 1 | | | | | |
| Osteogenesis imperfecta, type XV | 22408 | 7471 | WNT1 | 615220 | 1 |

### XI. Peripheral Nervous System (PNS) Diseases and Disorders

Provided herein are anti-TfR:Payload fusion proteins e.g., wherein the antigen-binding protein of the fusion (e.g., scFv) is 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263, and wherein the payload is a PNS disease or disorder therapeutic agent, e.g., a protein that is set forth below in Table J or a variant thereof. Methods for treating or preventing a PNS disease or disorder that is listed below in Table J, in a patient in need thereof, by administering an effective amount of an anti-TfR:Payload to the patient wherein the payload is a protein encoded by a gene corresponding to the particular PNS disease or disorder in Table J.

TABLE J
PNS Disease or Disorder and Corresponding Genes Encoding
Proteins which Can Be Fused to an Anti-TfR for Treatment.

| | Mouse gene | Human gene | Human | OMIM | Genes per |
|---|---|---|---|---|---|
| OMIM disease name | entrez id | entrez id | gene | id | OMIM disease |
| Visceral neuropathy, familial, 2, | 13866 | 2064 | ERBB2 | 619465 | 1 |
| autosomal recessive | | | | | |
| Arthrogryposis multiplex congenita | 243914 | 163175 | LGI4 | 617468 | 1 |
| 1, neurogenic, with myelin defect | | | | | |
| Multicentric osteolysis, nodulosis, | 17390 | 4313 | MMP2 | 259600 | 1 |
| and arthropathy | | | | | |
| Charcot-Marie-Tooth disease, type | 17988 | 10397 | NDRG1 | 601455 | 1 |
| 4D | | | | | |
| Hypogonadotropic hypogonadism | 18072 | 4808 | NHLH2 | 619755 | 1 |
| 27 without anosmia | | | | | |
| Charcot-Marie-Tooth disease, type | 225608 | 79628 | SH3TC2 | 601596 | 1 |
| 4C | | | | | |
| Neuropathy, hereditary motor and | 67453 | 91137 | SLC25A46 | 616505 | 1 |
| sensory, type VIB | | | | | |
| Pontocerebellar hypoplasia, type 1E | 67453 | 91137 | SLC25A46 | 619303 | 1 |
| Encephalopathy, progressive, with | 70430 | 6905 | TBCE | 617207 | 1 |
| amyotrophy and optic atrophy | | | | | |
| Hypoparathyroidism-retardation- | 70430 | 6905 | TBCE | 241410 | 1 |
| dysmorphism syndrome | | | | | |
| Kenny-Caffey syndrome, type 1 | 70430 | 6905 | TBCE | 244460 | 1 |

### XII. Skeletal Muscle Diseases and Disorders

Provided herein are anti-TfR:Payload fusion proteins e.g., wherein the antigen-binding protein of the fusion (e.g., scFv) is 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263, and wherein the payload is a skeletal muscle disease or disorder therapeutic agent, e.g., a protein that is set forth below in Table K or a variant thereof. Methods for treating or preventing a skeletal muscle disease or disorder that is listed below in Table K, in a patient in need thereof, by administering an effective amount of an anti-TfR:Payload to the patient wherein the payload is a protein encoded by a gene corresponding to the particular skeletal muscle disease or disorder in Table K.

TABLE K
Skeletal Muscle Disease or Disorder and Corresponding Genes Encoding
Proteins which Can Be Fused to an Anti-TfR for Treatment.

| | Mouse gene | Human gene | Human | OMIM | Genes per |
|---|---|---|---|---|---|
| OMIM disease name | entrez id | entrez id | gene | id | OMIM disease |
| Brody myopathy | 11937 | 487 | ATP2A1 | 601003 | 1 |
| Muscular dystrophy, limb-girdle, | 23828 | 11149 | BVES | 616812 | 1 |
| autosomal recessive 25 | | | | | |
| Lipodystrophy, congenital | 12389 | 857 | CAV1 | 612526 | 1 |
| generalized, type 3 | | | | | |
| Myasthenic syndrome, congenital, | 11435 | 1134 | CHRNA1 | 608930 | 1 |
| 1B, fast-channel | | | | | |
| Myasthenic syndrome, congenital, | 11447 | 1144 | CHRND | 616322 | 1 |
| 3B, fast-channel | | | | | |
| Myasthenic syndrome, congenital, | 11447 | 1144 | CHRND | 616323 | 1 |
| 3C, associated with acetylcholine | | | | | |
| receptor deficiency | | | | | |
| Ceroid lipofuscinosis, neuronal, 8 | 26889 | 2055 | CLN8 | 600143 | 1 |
| Ceroid lipofuscinosis, neuronal, 8, | 26889 | 2055 | CLN8 | 610003 | 1 |
| Northern epilepsy variant | | | | | |
| Spondylocarpotarsal synostosis | 286940 | 2317 | FLNB | 272460 | 1 |
| syndrome | | | | | |
| Hemolytic anemia due to glutathione | 14775 | 2876 | GPX1 | 614164 | 1 |
| peroxidase deficiency | | | | | |
| Gillespie syndrome | 16438 | 3708 | ITPR1 | 206700 | 1 |
| Nemaline myopathy 10 | 320502 | 56203 | LMOD3 | 616165 | 1 |
| Myopathy, congenital, progressive, | 18509 | 5081 | PAX7 | 618578 | 1 |
| with scoliosis | | | | | |
| Myasthenic syndrome, congenital, 16 | 110880 | 6329 | SCN4A | 614198 | 1 |
| Dystonia, dopa-responsive, due to | 20751 | 6697 | SPR | 612716 | 1 |
| sepiapterin reductase deficiency | | | | | |
| Split-hand/foot malformation 6 | 22410 | 7480 | WNT10B | 225300 | 1 |

### XIII. Cartilage Diseases and Disorders

Provided herein are anti-TfR:Payload fusion proteins e.g., wherein the antigen-binding protein of the fusion (e.g., scFv) is 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263, and wherein the payload is a cartilage disease or disorder therapeutic agent, e.g., a protein that is set forth below in Table L or a variant thereof. Methods for treating or preventing a cartilage disease or disorder that is listed below in Table L, in a patient in need thereof, by administering an effective amount of an anti-TfR:Payload to the patient wherein the payload is a protein encoded by a gene corresponding to the particular cartilage disease or disorder in Table L.

TABLE L
Cartilage Disease or Disorder and Corresponding Genes Encoding
Proteins which Can Be Fused to an Anti-TfR for Treatment.

| | Mouse gene | Human gene | Human | OMIM | Genes per |
|---|---|---|---|---|---|
| OMIM disease name | entrez id | entrez id | gene | id | OMIM disease |
| Spondyloepimetaphyseal dysplasia, | 11595 | 176 | ACAN | 612813 | 1 |
| aggrecan type | | | | | |
| Bardet-Biedl syndrome 2 | 67378 | 583 | BBS2 | 615981 | 1 |
| Retinitis pigmentosa 74 | 67378 | 583 | BBS2 | 616562 | 1 |
| Acromesomelic dysplasia 3 | 12167 | 658 | BMPR1B | 609441 | 1 |
| Osteochondrodysplasia, | 58250 | 50515 | CHST11 | 618167 | 1 |
| brachydactyly, and overlapping | | | | | |
| malformed digits | | | | | |
| Temtamy preaxial brachydactyly | 269941 | 22856 | CHSY1 | 605282 | 1 |
| syndrome | | | | | |
| Fibrochondrogenesis 1 | 12814 | 1301 | COL11A1 | 228520 | 1 |
| Deafness, autosomal recessive 53 | 12815 | 1302 | COL11A2 | 609706 | 1 |
| Fibrochondrogenesis 2 | 12815 | 1302 | COL11A2 | 614524 | 1 |
| Otospondylomegaepiphyseal | 12815 | 1302 | COL11A2 | 215150 | 1 |
| dysplasia, autosomal recessive | | | | | |
| Steel syndrome | 373864 | 85301 | COL27A1 | 615155 | 1 |
| Pycnodysostosis | 13038 | 1513 | CTSK | 265800 | 1 |
| Spondyloepimetaphyseal dysplasia, | 77006 | 65992 | DDRGK1 | 602557 | 1 |
| Shohat type | | | | | |
| Acromesomelic dysplasia 2A | 14563 | 8200 | GDF5 | 200700 | 1 |
| Acromesomelic dysplasia 2B | 14563 | 8200 | GDF5 | 228900 | 1 |
| Acromesomelic dysplasia 2C, | 14563 | 8200 | GDF5 | 201250 | 1 |
| Hunter-Thompson type | | | | | |
| Brachydactyly, type A1, C | 14563 | 8200 | GDF5 | 615072 | 1 |
| Leber congenital amaurosis 17 | 242316 | 392255 | GDF6 | 615360 | 1 |
| Short-rib thoracic dysplasia 2 with | 68259 | 57560 | IFT80 | 611263 | 1 |
| or without polydactyly | | | | | |
| Obesity, morbid, due to leptin | 16846 | 3952 | LEP | 614962 | 1 |
| deficiency | | | | | |
| Neurodevelopmental disorder with | 69605 | 80856 | LNPK | 618090 | 1 |
| epilepsy and hypoplasia of the | | | | | |
| corpus callosum | | | | | |
| Myopathy, congenital, progressive, | 18509 | 5081 | PAX7 | 618578 | 1 |
| with scoliosis | | | | | |
| Rhizomelic limb shortening with | 106522 | 91461 | PKDCC | 618821 | 1 |
| dysmorphic features | | | | | |
| Short stature, onychodysplasia, | 70235 | 25886 | POC1A | 614813 | 1 |
| facial dysmorphism, and | | | | | |
| hypotrichosis | | | | | |
| Hypoparathyroidism, familial | 19226 | 5741 | PTH | 146200 | 1 |
| isolated 1 | | | | | |
| Robinow syndrome, autosomal | 26564 | 4920 | ROR2 | 268310 | 1 |
| recessive | | | | | |
| Spondyloepimetaphyseal dysplasia, | 70661 | 23387 | SIK3 | 618162 | 1 |
| Krakow type | | | | | |
| Congenital disorder of | 67547 | 64116 | SLC39A8 | 616721 | 1 |
| glycosylation, type IIn | | | | | |
| Waardenburg syndrome, type 2D | 20583 | 6591 | SNAI2 | 608890 | 1 |

### XIV. Bone Growth Plate Diseases and Disorders

Provided herein are anti-TfR:Payload fusion proteins e.g., wherein the antigen-binding protein of the fusion (e.g., scFv) is 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263, and wherein the payload is a bone growth plate disease or disorder therapeutic agent, e.g., a protein that is set forth below in Table M or a variant thereof. Methods for treating or preventing a bone growth plate disease or disorder that is listed below in Table M, in a patient in need thereof, by administering an effective amount of an anti-TfR:Payload to the patient wherein the payload is a protein encoded by a gene corresponding to the particular bone growth plate disease or disorder in Table M.

TABLE M
Bone growth plate Disease or Disorder and Corresponding Genes Encoding
Proteins which Can Be Fused to an Anti-TfR for Treatment.

| OMIM | Mouse gene | Human gene | Human | OMIM | Genes per |
|---|---|---|---|---|---|
| disease name | entrez id | entrez id | gene | id | OMIM disease |
| Ehlers-Danlos | 12843 | 1278 | COL1A2 | 225320 | 1 |
| syndrome, | | | | | |
| cardiac | | | | | |
| valvular type | | | | | |
| Steel | 373864 | 85301 | COL27A1 | 615155 | 1 |
| syndrome | | | | | |
| Factor VII | 14068 | 2155 | F7 | 227500 | 1 |
| deficiency | | | | | |
| Short-rib | 68259 | 57560 | IFT80 | 611263 | 1 |
| thoracic | | | | | |
| dysplasia 2 | | | | | |
| with or | | | | | |
| without | | | | | |
| polydactyly | | | | | |
| Keratosis | 16971 | 4035 | LRP1 | 604093 | 1 |
| pilaris | | | | | |
| atrophicans | | | | | |
| Short stature, | 70235 | 25886 | POC1A | 614813 | 1 |
| onychodysplasia, | | | | | |
| facial | | | | | |
| dysmorphism, and | | | | | |
| hypotrichosis | | | | | |
| Nephrotic | 20397 | 8879 | SGPL1 | 617575 | 1 |
| syndrome, | | | | | |
| type 14 | | | | | |
| Waardenburg | 20583 | 6591 | SNAI2 | 608890 | 1 |
| syndrome, | | | | | |
| type 2D | | | | | |

### XV. Kidney Diseases and Disorders

Provided herein are anti-TfR:Payload fusion proteins e.g., wherein the antigen-binding protein of the fusion (e.g., scFv) is 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263, and wherein the payload is a kidney disease or disorder therapeutic agent, e.g., a protein that is set forth below in Table N or a variant thereof. Methods for treating or preventing a kidney disease or disorder that is listed below in Table N, in a patient in need thereof, by administering an effective amount of an anti-TfR:Payload to the patient wherein the payload is a protein encoded by a gene corresponding to the particular kidney disease or disorder in Table N.

TABLE N
Kidney Disease or Disorder and Corresponding Genes Encoding
Proteins which Can Be Fused to an Anti-TfR for Treatment.

| | Mouse gene | Human gene | Human | OMIM | Genes per |
|---|---|---|---|---|---|
| OMIM disease name | entrez id | entrez id | gene | id | OMIM disease |
| Cone-rod dystrophy 3 | 11304 | 24 | ABCA4 | 604116 | 1 |
| Fundus flavimaculatus | 11304 | 24 | ABCA4 | 248200 | 1 |
| Retinal dystrophy, early-onset | 11304 | 24 | ABCA4 | 248200 | 1 |
| severe | | | | | |
| Retinitis pigmentosa 19 | 11304 | 24 | ABCA4 | 601718 | 1 |
| Stargardt disease 1 | 11304 | 24 | ABCA4 | 248200 | 1 |
| Lethal congenital contracture | 11512 | 112 | ADCY6 | 616287 | 1 |
| syndrome 8 | | | | | |
| Nephronophthisis 16 | 75691 | 203286 | ANKS6 | 615382 | 1 |
| Distal renal tubular acidosis 3, | 140494 | 50617 | ATP6V0A4 | 602722 | 1 |
| with or without sensorineural | | | | | |
| hearing loss | | | | | |
| Distal renal tubular acidosis 2 with | 110935 | 525 | ATP6V1B1 | 267300 | 1 |
| progressive sensorineural hearing | | | | | |
| loss | | | | | |
| Bardet-Biedl syndrome 2 | 67378 | 583 | BBS2 | 615981 | 1 |
| Retinitis pigmentosa 74 | 67378 | 583 | BBS2 | 616562 | 1 |
| Deafness, autosomal recessive 93 | 29866 | 51475 | CABP2 | 614899 | 1 |
| Cone-rod synaptic disorder, | 73660 | 57010 | CABP4 | 610427 | 1 |
| congenital nonprogressive | | | | | |
| Joubert syndrome 5 | 216274 | 80184 | CEP290 | 610188 | 1 |
| Leber congenital amaurosis 10 | 216274 | 80184 | CEP290 | 611755 | 1 |
| Meckel syndrome 4 | 216274 | 80184 | CEP290 | 611134 | 1 |
| Senior-Loken syndrome 6 | 216274 | 80184 | CEP290 | 610189 | 1 |
| Bartter syndrome, type 3 | 56365 | 1188 | CLCNKB | 607364 | 1 |
| Deafness, autosomal recessive 103 | 224796 | 53405 | CLIC5 | 616042 | 1 |
| Ceroid lipofuscinosis, neuronal, | 76524 | 54982 | CLN6 | 601780 | 1 |
| 6A | | | | | |
| Ceroid lipofuscinosis, neuronal, | 76524 | 54982 | CLN6 | 204300 | 1 |
| 6B (Kufs type) | | | | | |
| Ceroid lipofuscinosis, neuronal, 8 | 26889 | 2055 | CLN8 | 600143 | 1 |
| Ceroid lipofuscinosis, neuronal, 8, | 26889 | 2055 | CLN8 | 610003 | 1 |
| Northern epilepsy variant | | | | | |
| Retinitis pigmentosa 61 | 229320 | 7401 | CLRN1 | 614180 | 1 |
| Usher syndrome, type 3A | 229320 | 7401 | CLRN1 | 276902 | 1 |
| Achromatopsia 2 | 12790 | 1261 | CNGA3 | 216900 | 1 |
| Fibrochondrogenesis 1 | 12814 | 1301 | COL11A1 | 228520 | 1 |
| Joubert syndrome 17 | 73692 | 65250 | CPLANE1 | 614615 | 1 |
| Orofaciodigital syndrome VI | 73692 | 65250 | CPLANE1 | 277170 | 1 |
| Leber congenital amaurosis 7 | 12951 | 1406 | CRX | 613829 | 1 |
| Cataract 22 | 12962 | 1417 | CRYBB3 | 609741 | 1 |
| Chronic granulomatous disease 4, | 13057 | 1535 | CYBA | 233690 | 1 |
| autosomal recessive | | | | | |
| Cone-rod dystrophy 21 | 67171 | 128338 | DRAM2 | 616502 | 1 |
| Short-rib thoracic dysplasia 3 with | 110350 | 79659 | DYNC2H1 | 613091 | 1 |
| or without polydactyly | | | | | |
| Leber congenital amaurosis 17 | 242316 | 392255 | GDF6 | 615360 | 1 |
| Hyperekplexia 2 | 14658 | 2743 | GLRB | 614619 | 1 |
| Night blindness, congenital | 108072 | 2916 | GRM6 | 257270 | 1 |
| stationary (complete), 1B, | | | | | |
| autosomal recessive | | | | | |
| Immunodeficiency-centromeric | 15201 | 3070 | HELLS | 616911 | 1 |
| instability-facial anomalies | | | | | |
| syndrome 4 | | | | | |
| Muscular dystrophy, congenital, | 19062 | 51763 | INPP5K | 617404 | 1 |
| with cataracts and intellectual | | | | | |
| disability | | | | | |
| Renal hypodysplasia/aplasia 1 | 241226 | 8516 | ITGA8 | 191830 | 1 |
| SESAME syndrome | 16513 | 3766 | KCNJ10 | 612780 | 1 |
| Cerebellar atrophy, developmental | 16531 | 3778 | KCNMA1 | 617643 | 1 |
| delay, and seizures | | | | | |
| Pseudohypoaldosteronism, type | 100503085 | 26249 | KLHL3 | 614495 | 1 |
| IID | | | | | |
| Cortical malformations, occipital | 23928 | 10319 | LAMC3 | 614115 | 1 |
| Leber congenital amaurosis 14 | 79235 | 9227 | LRAT | 613341 | 1 |
| Retinal dystrophy, early-onset | 79235 | 9227 | LRAT | 613341 | 1 |
| severe | | | | | |
| Retinitis pigmentosa, juvenile | 79235 | 9227 | LRAT | 613341 | 1 |
| Night blindness, congenital | 242235 | 345193 | LRIT3 | 615058 | 1 |
| stationary (complete), 1F, | | | | | |
| autosomal recessive | | | | | |
| Metaphyseal anadysplasia 2 | 17395 | 4318 | MMP9 | 613073 | 1 |
| Deafness, autosomal recessive 30 | 667663 | 53904 | MYO3A | 607101 | 1 |
| Deafness, autosomal recessive 2 | 17921 | 4647 | MYO7A | 600060 | 1 |
| Usher syndrome, type 1B | 17921 | 4647 | MYO7A | 276900 | 1 |
| Short-rib thoracic dysplasia 6 with | 18004 | 4750 | NEK1 | 263520 | 1 |
| or without polydactyly | | | | | |
| Meckel syndrome 7 | 74025 | 27031 | NPHP3 | 267010 | 1 |
| Nephronophthisis 3 | 74025 | 27031 | NPHP3 | 604387 | 1 |
| Renal-hepatic-pancreatic dysplasia | 74025 | 27031 | NPHP3 | 208540 | 1 |
| 1 | | | | | |
| Boudin-Mortier syndrome | 18162 | 4883 | NPR3 | 619543 | 1 |
| Microcephalic osteodysplastic | 18541 | 5116 | PCNT | 210720 | 1 |
| primordial dwarfism, type II | | | | | |
| Retinitis pigmentosa 43 | 225600 | 5145 | PDE6A | 613810 | 1 |
| Retinitis pigmentosa-40 | 18587 | 5158 | PDE6B | 613801 | 1 |
| Leber congenital amaurosis 12 | 74023 | 343035 | RD3 | 610612 | 1 |
| Bothnia retinal dystrophy | 19771 | 6017 | RLBP1 | 607475 | 1 |
| Newfoundland rod-cone dystrophy | 19771 | 6017 | RLBP1 | 607476 | 1 |
| COACH syndrome 3 | 244585 | 23322 | RPGRIP1L | 619113 | 1 |
| Joubert syndrome 7 | 244585 | 23322 | RPGRIP1L | 611560 | 1 |
| Meckel syndrome 5 | 244585 | 23322 | RPGRIP1L | 611561 | 1 |
| Nephrotic syndrome, type 14 | 20397 | 8879 | SGPL1 | 617575 | 1 |
| Leber congenital amaurosis 3 | 104871 | 55812 | SPATA7 | 604232 | 1 |
| Retinitis pigmentosa 94, variable | 104871 | 55812 | SPATA7 | 604232 | 1 |
| age at onset, autosomal recessive | | | | | |
| Immunodeficiency 31B, | 20846 | 6772 | STAT1 | 613796 | 1 |
| mycobacterial and viral infections, | | | | | |
| autosomal recessive | | | | | |
| Corneal dystrophy, gelatinous | 56753 | 4070 | TACSTD2 | 204870 | 1 |
| drop-like | | | | | |
| Segawa syndrome, recessive | 21823 | 7054 | TH | 605407 | 1 |
| COACH syndrome 1 | 329795 | 91147 | TMEM67 | 216360 | 1 |
| Joubert syndrome 6 | 329795 | 91147 | TMEM67 | 610688 | 1 |
| Meckel syndrome 3 | 329795 | 91147 | TMEM67 | 607361 | 1 |
| Nephronophthisis 11 | 329795 | 91147 | TMEM67 | 613550 | 1 |
| RHYNS syndrome | 329795 | 91147 | TMEM67 | 602152 | 1 |
| Night blindness, congenital | 17364 | 4308 | TRPM1 | 613216 | 1 |
| stationary (complete), 1C, | | | | | |
| autosomal recessive | | | | | |
| Diarrhea 9 | 22414 | 7482 | WNT2B | 618168 | 1 |
| Nephronophthisis-like | 321003 | 63929 | XPNPEP3 | 613159 | 1 |
| nephropathy 1 | | | | | |

### XVI. Blood Diseases and Disorders

Provided herein are anti-TfR:Payload fusion proteins e.g., wherein the antigen-binding protein of the fusion (e.g., scFv) is 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263, and wherein the payload is a blood disease or disorder therapeutic agent, e.g., a protein that is set forth below in Table O or a variant thereof. Methods for treating or preventing a blood disease or disorder that is listed below in Table O, in a patient in need thereof, by administering an effective amount of an anti-TfR:Payload to the patient wherein the payload is a protein encoded by a gene corresponding to the particular blood

TABLE O
Blood Disease or Disorder and Corresponding Genes Encoding
Proteins which Can Be Fused to an Anti-TfR for Treatment.

| | Mouse gene | Human gene | Human | OMIM | Genes per |
|---|---|---|---|---|---|
| OMIM disease name | entrez id | entrez id | gene | id | OMIM disease |
| Combined oxidative | 224805 | 57505 | AARS2 | 614096 | 1 |
| phosphorylation deficiency 8 | | | | | |
| Leukoencephalopathy, | 224805 | 57505 | AARS2 | 615889 | 1 |
| progressive, with ovarian failure | | | | | |
| 2-methylbutyrylglycinuria | 66885 | 36 | ACADSB | 610006 | 1 |
| Alpha-methylacetoacetic aciduria | 110446 | 38 | ACAT1 | 203750 | 1 |
| Aicardi-Goutieres syndrome 6 | 56417 | 103 | ADAR | 615010 | 1 |
| Neurodevelopmental disorder with | 110532 | 104 | ADARB1 | 618862 | 1 |
| hypotonia, microcephaly, and | | | | | |
| seizures | | | | | |
| Deafness, autosomal recessive 44 | 432530 | 107 | ADCY1 | 610154 | 1 |
| Obesity, susceptibility to, | 104111 | 109 | ADCY3 | 617885 | 1 |
| BMIQ19} | | | | | |
| Lethal congenital contracture | 11512 | 112 | ADCY6 | 616287 | 1 |
| syndrome 8 | | | | | |
| Hypermethioninemia due to | 11534 | 132 | ADK | 614300 | 1 |
| adenosine kinase deficiency | | | | | |
| Neurodegeneration, childhood- | 100206 | 54936 | ADPRS | 618170 | 1 |
| onset, stress-induced, with variable | | | | | |
| ataxia and seizures | | | | | |
| Alopecia-intellectual disability | 11625 | 197 | AHSG | 203650 | 1 |
| syndrome 1 | | | | | |
| Immunodeficiency with hyper- | 11628 | 57379 | AICDA | 605258 | 1 |
| IgM, type 2 | | | | | |
| Leukodystrophy, hypomyelinating, | 13722 | 9255 | AIMP1 | 260600 | 1 |
| 3 | | | | | |
| Autoimmune polyendocrinopathy | 11634 | 326 | AIRE | 240300 | 1 |
| syndrome, type I, with or without | | | | | |
| reversible metaphyseal dysplasia | | | | | |
| Spermatogenic failure 27 | 78801 | 122481 | AK7 | 617965 | 1 |
| Glycogen storage disease XII | 11674 | 226 | ALDOA | 611881 | 1 |
| Fructose intolerance, hereditary | 230163 | 229 | ALDOB | 229600 | 1 |
| Intellectual developmental | 67667 | 91801 | ALKBH8 | 618504 | 1 |
| disorder, autosomal recessive 71 | | | | | |
| Myopathy due to myoadeny late | 229665 | 270 | AMPD1 | 615511 | 1 |
| deaminase deficiency | | | | | |
| Pontocerebellar hypoplasia, type 9 | 109674 | 271 | AMPD2 | 615809 | 1 |
| Spastic paraplegia 63 | 109674 | 271 | AMPD2 | 615686 | 1 |
| Ferguson-Bonni | 56317 | 51434 | ANAPC7 | 619699 | 1 |
| neurodevelopmental syndrome | | | | | |
| Scott syndrome | 105722 | 196527 | ANO6 | 262890 | 1 |
| Spastic paraplegia 48, autosomal | 231855 | 9907 | AP5Z1 | 613647 | 1 |
| recessive | | | | | |
| Adenine phosphoribosyltransferase | 11821 | 353 | APRT | 614723 | 1 |
| deficiency | | | | | |
| Ataxia, early-onset, with | 66408 | 54840 | APTX | 208920 | 1 |
| oculomotor apraxia and | | | | | |
| hypoalbuminemia | | | | | |
| Spinal muscular atrophy with | 69090 | 51008 | ASCC1 | 616867 | 1 |
| congenital bone fractures 2 | | | | | |
| Cutis laxa, autosomal recessive, | 11964 | 523 | ATP6V1A | 617403 | 1 |
| type IID | | | | | |
| Distal renal tubular acidosis 2 with | 110935 | 525 | ATP6V1B1 | 267300 | 1 |
| progressive sensorineural hearing | | | | | |
| loss | | | | | |
| Muscular dystrophy - | 97884 | 148789 | B3GALNT2 | 615181 | 1 |
| dystroglycanopathy (congenital | | | | | |
| with brain and eye anomalies, type | | | | | |
| A, 11 | | | | | |
| Bile acid conjugation defect 1 | 12012 | 570 | BAAT | 619232 | 1 |
| Agammaglobulinemia 4 | 17060 | 29760 | BLNK | 613502 | 1 |
| Hermansky-Pudlak syndrome 9 | 18457 | 26258 | BLOC1S6 | 614171 | 1 |
| Acromesomelic dysplasia 3 | 12167 | 658 | BMPR1B | 609441 | 1 |
| Erythrocytosis, familial, 8 | 12183 | 669 | BPGM | 222800 | 1 |
| Fanconi anemia, complementation | 237911 | 83990 | BRIP1 | 609054 | 1 |
| group J | | | | | |
| Desbuquois dysplasia 1 | 76025 | 124583 | CANT1 | 251450 | 1 |
| Epiphyseal dysplasia, multiple, 7 | 76025 | 124583 | CANT1 | 617719 | 1 |
| Immunodeficiency 11A | 108723 | 84433 | CARD11 | 615206 | 1 |
| Immunodeficiency, common | 12478 | 930 | CD19 | 613493 | 1 |
| variable, 3 | | | | | |
| Lymphoproliferative syndrome 2 | 21940 | 939 | CD27 | 615122 | 1 |
| Immunodeficiency with hyper- | 21939 | 958 | CD40 | 606843 | 1 |
| IgM, type 3 | | | | | |
| Deafness, autosomal recessive 32, | 229776 | 8556 | CDC14A | 608653 | 1 |
| with or without immotile sperm | | | | | |
| Microcephaly 12, primary, | 12571 | 1021 | CDK6 | 616080 | 1 |
| autosomal recessive | | | | | |
| Microcephaly 13, primary, | 229841 | 1062 | CENPE | 616051 | 1 |
| autosomal recessive | | | | | |
| Nephronophthisis 15 | 214552 | 22897 | CEP164 | 614845 | 1 |
| Complement factor B deficiency | 14962 | 629 | CFB | 615561 | 1 |
| Cocoon syndrome | 12675 | 1147 | CHUK | 613630 | 1 |
| Popliteal pterygium syndrome, | 12675 | 1147 | CHUK | 619339 | 1 |
| Bartsocas-Papas type 2 | | | | | |
| Cold-induced sweating syndrome | 56708 | 23529 | CLCF1 | 610313 | 1 |
| 2 | | | | | |
| Leukodystrophy, hypomyelinating, | 12799 | 1267 | CNP | 619071 | 1 |
| 20 | | | | | |
| Pitt-Hopkins like syndrome 1 | 66797 | 26047 | CNTNAP2 | 610042 | 1 |
| Neurodegeneration with brain iron | 71743 | 80347 | COASY | 615643 | 1 |
| accumulation 6 | | | | | |
| Pontocerebellar hypoplasia, type | 71743 | 80347 | COASY | 618266 | 1 |
| 12 | | | | | |
| Carbamoylphosphate synthetase I | 227231 | 1373 | CPS1 | 237300 | 1 |
| deficiency | | | | | |
| Surfactant metabolism | 12983 | 1439 | CSF2RB | 614370 | 1 |
| dysfunction, pulmonary, 5 | | | | | |
| Neutropenia, severe congenital, 7, | 12986 | 1441 | CSF3R | 617014 | 1 |
| autosomal recessive | | | | | |
| Joubert syndrome 21 | 211660 | 79848 | CSPP1 | 615636 | 1 |
| Cerebroretinal microangiopathy | 68964 | 80169 | CTC1 | 612199 | 1 |
| with calcifications and cysts | | | | | |
| Microcephaly, facial | 66965 | 348180 | CTU2 | 618142 | 1 |
| dysmorphism, renal agenesis, and | | | | | |
| ambiguous genitalia syndrome | | | | | |
| WHIM syndrome 2 | 12765 | 3579 | CXCR2 | 619407 | 1 |
| Aromatase deficiency | 13075 | 1588 | CYP19A1 | 613546 | 1 |
| Bile acid synthesis defect, | 13123 | 9420 | CYP7B1 | 613812 | 1 |
| congenital, 3 | | | | | |
| Spastic paraplegia 5A, autosomal | 13123 | 9420 | CYP7B1 | 270800 | 1 |
| recessive | | | | | |
| Developmental and epileptic | 67789 | 55152 | DALRD3 | 618910 | 1 |
| encephalopathy 86 | | | | | |
| Leukoencephalopathy with brain | 226539 | 55157 | DARS2 | 611105 | 1 |
| stem and spinal cord involvement | | | | | |
| and lactate elevation | | | | | |
| Oculocutaneous albinism, type | 13190 | 1638 | DCT | 619165 | 1 |
| VIII | | | | | |
| Pentosuria] | 67880 | 51181 | DCXR | 260800 | 1 |
| Aromatic L-amino acid | 13195 | 1644 | DDC | 608643 | 1 |
| decarboxylase deficiency | | | | | |
| Mitochondrial DNA depletion | 27369 | 1716 | DGUOK | 251880 | 1 |
| syndrome 3 (hepatocerebral type) | | | | | |
| Progressive external | 27369 | 1716 | DGUOK | 617070 | 1 |
| ophthalmoplegia with | | | | | |
| mitochondrial DNA deletions, | | | | | |
| autosomal recessive 4 | | | | | |
| Miller syndrome | 56749 | 1723 | DHODH | 263750 | 1 |
| Pyruvate dehydrogenase E2 | 235339 | 1737 | DLAT | 245348 | 1 |
| deficiency | | | | | |
| Systemic lupus erythematosus 16 | 13421 | 1776 | DNASE1L3 | 614420 | 1 |
| Immunodeficiency-centromeric | 13436 | 1789 | DNMT3B | 242860 | 1 |
| instability-facial anomalies | | | | | |
| syndrome 1 | | | | | |
| Immunodeficiency 40 | 94176 | 1794 | DOCK2 | 616433 | 1 |
| Congenital disorder of | 13480 | 8813 | DPM1 | 608799 | 1 |
| glycosylation, type Ie | | | | | |
| 5-fluorouracil toxicity | 99586 | 1806 | DPYD | 274270 | 1 |
| Dihydropyrimidine dehydrogenase | 99586 | 1806 | DPYD | 274270 | 1 |
| deficiency | | | | | |
| Intellectual developmental | 353190 | 80153 | EDC3 | 616460 | 1 |
| disorder, autosomal recessive 50 | | | | | |
| Combined oxidative | 68626 | 60528 | ELAC2 | 615440 | 1 |
| phosphorylation deficiency 17 | | | | | |
| Dysautonomia, familial | 230233 | 8518 | ELP1 | 223900 | 1 |
| Spastic paraplegia 64, autosomal | 12495 | 953 | ENTPD1 | 615683 | 1 |
| recessive | | | | | |
| Bleeding disorder, platelet-type, 22 | 13844 | 2048 | EPHB2 | 618462 | 1 |
| Eosinophil peroxidase deficiency] | 13861 | 8288 | EPX | 261500 | 1 |
| Visceral neuropathy, familial, 2, | 13866 | 2064 | ERBB2 | 619465 | 1 |
| autosomal recessive | | | | | |
| Fanconi anemia, complementation | 50505 | 2072 | ERCC4 | 615272 | 1 |
| group Q | | | | | |
| XFE progeroid syndrome | 50505 | 2072 | ERCC4 | 610965 | 1 |
| Xeroderma pigmentosum, group F | 50505 | 2072 | ERCC4 | 278760 | 1 |
| Xeroderma pigmentosum, type | 50505 | 2072 | ERCC4 | 278760 | 1 |
| F/Cockayne syndrome | | | | | |
| Cerebrooculofacioskeletal | 22592 | 2073 | ERCC5 | 616570 | 1 |
| syndrome 3 | | | | | |
| Xeroderma pigmentosum, group G | 22592 | 2073 | ERCC5 | 278780 | 1 |
| Xeroderma pigmentosum, group | 22592 | 2073 | ERCC5 | 278780 | 1 |
| G/Cockayne syndrome | | | | | |
| Cockayne syndrome, type A | 71991 | 1161 | ERCC8 | 216400 | 1 |
| UV-sensitive syndrome 2 | 71991 | 1161 | ERCC8 | 614621 | 1 |
| Deafness, autosomal recessive 109 | 207920 | 54845 | ESRP1 | 618013 | 1 |
| Pontocerebellar hypoplasia, type | 66583 | 51013 | EXOSC1 | 619304 | 1 |
| 1F | | | | | |
| Dysprothrombinemia | 14061 | 2147 | F2 | 613679 | 1 |
| Hypoprothrombinemia | 14061 | 2147 | F2 | 613679 | 1 |
| Immunodeficiency 90 with | 14082 | 8772 | FADD | 613759 | 1 |
| encephalopathy, functional | | | | | |
| hyposplenia, and hepatic | | | | | |
| dysfunction | | | | | |
| Raine syndrome | 80752 | 56975 | FAM20C | 259775 | 1 |
| Fanconi anemia, complementation | 211651 | 2177 | FANCD2 | 227646 | 1 |
| group D2 | | | | | |
| Fanconi anemia, complementation | 208836 | 55215 | FANCI | 609053 | 1 |
| group I | | | | | |
| Fanconi anemia, complementation | 67030 | 55120 | FANCL | 614083 | 1 |
| group L | | | | | |
| Peroxisomal fatty acyl-CoA | 67420 | 84188 | FAR1 | 616154 | 1 |
| reductase 1 disorder | | | | | |
| Combined oxidative | 69955 | 10667 | FARS2 | 614946 | 1 |
| phosphorylation deficiency 14 | | | | | |
| Spastic paraplegia 77, autosomal | 69955 | 10667 | FARS2 | 617046 | 1 |
| recessive | | | | | |
| Rajab interstitial lung disease with | 66590 | 2193 | FARSA | 619013 | 1 |
| brain calcifications 2 | | | | | |
| Combined oxidative | 75619 | 22868 | FASTKD2 | 618855 | 1 |
| phosphorylation deficiency 44 | | | | | |
| Parkinson disease 15, autosomal | 69754 | 25793 | FBXO7 | 260300 | 1 |
| recessive | | | | | |
| Leukocyte adhesion deficiency, | 108101 | 83706 | FERMT3 | 612840 | 1 |
| type III | | | | | |
| Siddiqi syndrome | 228859 | 128486 | FITM2 | 618635 | 1 |
| Anterior segment dysgenesis 2, | 30923 | 2301 | FOXE3 | 610256 | 1 |
| multiple subtypes | | | | | |
| T-cell immunodeficiency, | 15218 | 8456 | FOXN1 | 601705 | 1 |
| congenital alopecia, and nail | | | | | |
| dystrophy | | | | | |
| Combined oxidative | 229487 | 5188 | GATB | 618838 | 1 |
| phosphorylation deficiency 41 | | | | | |
| Glutaricaciduria, type I | 270076 | 2639 | GCDH | 231670 | 1 |
| Diabetes mellitus, permanent | 103988 | 2645 | GCK | 606176 | 1 |
| neonatal 1 | | | | | |
| Bleeding disorder, platelet-type, 17 | 14582 | 8328 | GFI1B | 187900 | 1 |
| Nonaka myopathy | 50798 | 10020 | GNE | 605820 | 1 |
| Hypertriglyceridemia, transient | 14555 | 2819 | GPD1 | 614480 | 1 |
| infantile | | | | | |
| Chudley-McCullough syndrome | 76123 | 29899 | GPSM2 | 604213 | 1 |
| Jaberi-Elahi syndrome | 56055 | 54676 | GTPBP2 | 617988 | 1 |
| Combined oxidative | 70359 | 84705 | GTPBP3 | 616198 | 1 |
| phosphorylation deficiency 23 | | | | | |
| Vertebral, cardiac, renal, and limb | 107766 | 23498 | HAAO | 617660 | 1 |
| defects syndrome 1 | | | | | |
| T-cell lymphoma, subcutaneous | 171285 | 84868 | HAVCR2 | 618398 | 1 |
| panniculitis-like | | | | | |
| Immunodeficiency-centromeric | 15201 | 3070 | HELLS | 616911 | 1 |
| instability-facial anomalies | | | | | |
| syndrome 4 | | | | | |
| Hemochromatosis, type 2A | 69585 | 148738 | HJV | 602390 | 1 |
| Heme oxygenase-1 deficiency | 15368 | 3162 | HMOX1 | 614034 | 1 |
| Dystonia 2, torsion, autosomal | 15444 | 3208 | HPCA | 224500 | 1 |
| recessive | | | | | |
| D-bifunctional protein deficiency | 15488 | 3295 | HSD17B4 | 261515 | 1 |
| Perrault syndrome 1 | 15488 | 3295 | HSD17B4 | 233400 | 1 |
| Premature ovarian failure 19 | 74377 | 11077 | HSF2BP | 619245 | 1 |
| Immunodeficiency 27A, | 15979 | 3459 | IFNGR1 | 209950 | 1 |
| mycobacteriosis, AR | | | | | |
| Charcot-Marie-Tooth disease, | 20589 | 3508 | IGHMBP2 | 616155 | 1 |
| axonal, type 2S | | | | | |
| Neuronopathy, distal hereditary | 20589 | 3508 | IGHMBP2 | 604320 | 1 |
| motor, type VI | | | | | |
| Immunodeficiency 15B | 16150 | 3551 | IKBKB | 615592 | 1 |
| Immunodeficiency 29, | 16160 | 3593 | IL12B | 614890 | 1 |
| mycobacteriosis | | | | | |
| Immunodeficiency 30 | 16161 | 3594 | IL12RB1 | 614891 | 1 |
| Candidiasis, familial, 9 | 171095 | 84818 | IL17RC | 616445 | 1 |
| Immunodeficiency, common | 60505 | 59067 | IL21 | 615767 | 1 |
| variable, 11 | | | | | |
| Immunodeficiency 56 | 60504 | 50615 | IL21R | 615207 | 1 |
| Immunodeficiency 41 with | 16184 | 3559 | IL2RA | 606367 | 1 |
| lymphoproliferation and | | | | | |
| auto immunity | | | | | |
| Immunodeficiency 63 with | 16185 | 3560 | IL2RB | 618495 | 1 |
| lymphoproliferation and | | | | | |
| autoimmunity | | | | | |
| Immunodeficiency 39 | 54123 | 3665 | IRF7 | 616345 | 1 |
| Immunodeficiency 32B, monocyte | 15900 | 3394 | IRF8 | 226990 | 1 |
| and dendritic cell deficiency, | | | | | |
| autosomal recessive | | | | | |
| Autoimmune disease, multisystem, | 16396 | 83737 | ITCH | 613385 | 1 |
| with facial dysmorphism | | | | | |
| Lymphoproliferative syndrome 1 | 16428 | 3702 | ITK | 613011 | 1 |
| Muscular dystrophy, limb-girdle, | 16450 | 3714 | JAG2 | 619566 | 1 |
| autosomal recessive 27 | | | | | |
| SCID, autosomal recessive, T- | 16453 | 3718 | JAK3 | 600802 | 1 |
| negative/B-positive type | | | | | |
| Basal ganglia calcification, | 67374 | 58494 | JAM2 | 618824 | 1 |
| idiopathic, 8, autosomal recessive | | | | | |
| Hemorrhagic destruction of the | 83964 | 83700 | JAM3 | 613730 | 1 |
| brain, subependymal calcification, | | | | | |
| and cataracts | | | | | |
| Hydroxykynureninuria | 70789 | 8942 | KYNU | 236800 | 1 |
| Vertebral, cardiac, renal, and limb | 70789 | 8942 | KYNU | 617661 | 1 |
| defects syndrome 2 | | | | | |
| Immunodeficiency 52 | 16797 | 27040 | LAT | 617514 | 1 |
| Immunodeficiency 81 | 16822 | 3937 | LCP2 | 619374 | 1 |
| Obesity, morbid, due to leptin | 16846 | 3952 | LEP | 614962 | 1 |
| deficiency | | | | | |
| Obesity, morbid, due to leptin | 16847 | 3953 | LEPR | 614963 | 1 |
| receptor deficiency | | | | | |
| Lipodystrophy, familial partial, | 16890 | 3991 | LIPE | 615980 | 1 |
| type 6 | | | | | |
| Chediak-Higashi syndrome | 17101 | 1130 | LYST | 214500 | 1 |
| 3-Methylcrotonyl-CoA | 78038 | 64087 | MCCC2 | 210210 | 1 |
| carboxylase 2 deficiency | | | | | |
| Basel-Vanagait-Smirin-Yosef | 75613 | 81857 | MED25 | 616449 | 1 |
| syndrome | | | | | |
| Intellectual developmental | 75422 | 29081 | METTL5 | 618665 | 1 |
| disorder, autosomal recessive 72 | | | | | |
| Mitochondrial DNA depletion | 74528 | 92667 | MGME1 | 615084 | 1 |
| syndrome 11 | | | | | |
| Mismatch repair cancer syndrome | 17350 | 4292 | MLH1 | 276300 | 1 |
| 1 | | | | | |
| Metaphyseal anadysplasia 2 | 17395 | 4318 | MMP9 | 613073 | 1 |
| Xanthinuria, type II | 68591 | 55034 | MOCOS | 603592 | 1 |
| Molybdenum cofactor deficiency | 17434 | 4338 | MOCS2 | 252160 | 1 |
| B | | | | | |
| Thrombocytopenia, anemia, and | 106722 | 80739 | MPIG6B | 617441 | 1 |
| myelofibrosis | | | | | |
| Deafness, autosomal recessive 111 | 14012 | 10205 | MPZL2 | 618145 | 1 |
| Familial adenomatous polyposis 4 | 17686 | 4437 | MSH3 | 617100 | 1 |
| Premature ovarian failure 13 | 17687 | 4439 | MSH5 | 617442 | 1 |
| Vertebral, cardiac, renal, and limb | 78914 | 55191 | NADSYN1 | 618845 | 1 |
| defects syndrome 3 | | | | | |
| Encephalopathy, progressive, | 246703 | 128240 | NAXE | 617186 | 1 |
| early-onset, with brain edema | | | | | |
| and/or leukoencephalopathy | | | | | |
| Infantile liver failure syndrome 2 | 71169 | 51594 | NBAS | 616483 | 1 |
| Short stature, optic nerve atrophy, | 71169 | 51594 | NBAS | 614800 | 1 |
| and Pelger-Huet anomaly | | | | | |
| Microcephaly 22, primary, | 78658 | 23310 | NCAPD3 | 617984 | 1 |
| autosomal recessive | | | | | |
| Chronic granulomatous disease 1, | 17969 | 653361 | NCF1 | 233700 | 1 |
| autosomal recessive | | | | | |
| Chronic granulomatous disease 2, | 17970 | 4688 | NCF2 | 233710 | 1 |
| autosomal recessive | | | | | |
| Charcot-Marie-Tooth disease, type | 17988 | 10397 | NDRG1 | 601455 | 1 |
| 4D | | | | | |
| Mitochondrial complex I | 67273 | 4705 | NDUFA10 | 618243 | 1 |
| deficiency, nuclear type 22 | | | | | |
| Mitochondrial complex I | 68375 | 4702 | NDUFA8 | 619272 | 1 |
| deficiency, nuclear type 37 | | | | | |
| Mitochondrial complex I | 67264 | 4714 | NDUFB8 | 618252 | 1 |
| deficiency, nuclear type 32 | | | | | |
| Mitochondrial complex I | 66218 | 4715 | NDUFB9 | 618245 | 1 |
| deficiency, nuclear type 24 | | | | | |
| Mitochondrial complex I | 227197 | 4719 | NDUFS1 | 618226 | 1 |
| deficiency, nuclear type 5 | | | | | |
| Dyskeratosis congenita, autosomal | 52530 | 55651 | NHP2 | 613987 | 1 |
| recessive 2 | | | | | |
| Glucocorticoid deficiency 4, with | 18115 | 23530 | NNT | 614736 | 1 |
| or without mineralocorticoid | | | | | |
| deficiency | | | | | |
| Acromesomelic dysplasia 1, | 230103 | 4882 | NPR2 | 602875 | 1 |
| Maroteaux type | | | | | |
| Boudin-Mortier syndrome | 18162 | 4883 | NPR3 | 619543 | 1 |
| Spastic paraplegia 45, autosomal | 76952 | 22978 | NT5C2 | 613162 | 1 |
| recessive | | | | | |
| Insensitivity to pain, congenital, | 18211 | 4914 | NTRK1 | 256800 | 1 |
| with anhidrosis | | | | | |
| Striatonigral degeneration, | 18226 | 23636 | NUP62 | 271930 | 1 |
| infantile | | | | | |
| Nephrotic syndrome, type 17 | 445007 | 79902 | NUP85 | 618176 | 1 |
| Oxoglutarate dehydrogenase | 18293 | 4967 | OGDH | 203740 | 1 |
| deficiency | | | | | |
| Hyperphenylalaninemia, non-PKU | 18478 | 5053 | PAH | 261600 | 1 |
| mild] | | | | | |
| Phenylketonuria | 18478 | 5053 | PAH | 261600 | 1 |
| Parkinson disease 7, autosomal | 57320 | 11315 | PARK7 | 606324 | 1 |
| recessive early-onset | | | | | |
| Myopathy, congenital, progressive, | 18509 | 5081 | PAX7 | 618578 | 1 |
| with scoliosis | | | | | |
| Intellectual developmental disorder | 207728 | 5138 | PDE2A | 619150 | 1 |
| with paroxysmal dyskinesia or | | | | | |
| seizures | | | | | |
| Lacticacidemia due to PDX1 | 27402 | 8050 | PDHX | 245349 | 1 |
| deficiency | | | | | |
| Pancreatic agenesis 1 | 18609 | 3651 | PDX1 | 260370 | 1 |
| Neuropathy, hereditary motor and | 216134 | 8566 | PDXK | 618511 | 1 |
| sensory, type VIC, with optic | | | | | |
| atrophy | | | | | |
| Glycogen storage disease VII | 18642 | 5213 | PFKM | 232800 | 1 |
| Immunodeficiency 23 | 109785 | 5238 | PGM3 | 615816 | 1 |
| Rhizomelic limb shortening with | 106522 | 91461 | PKDCC | 618821 | 1 |
| dysmorphic features | | | | | |
| Developmental and epileptic | 18795 | 23236 | PLCB1 | 613722 | 1 |
| encephalopathy 12 | | | | | |
| Osteopetrosis, autosomal recessive | 353047 | 9842 | PLEKHM1 | 611497 | 1 |
| 6 | | | | | |
| Short stature, onychodysplasia, | 70235 | 25886 | POC1A | 614813 | 1 |
| facial dysmorphism, and | | | | | |
| hypotrichosis | | | | | |
| Mitochondrial DNA depletion | 50776 | 11232 | POLG2 | 618528 | 1 |
| syndrome 16 (hepatic type) | | | | | |
| Mitochondrial DNA depletion | 50776 | 11232 | POLG2 | 619425 | 1 |
| syndrome 16B (neuroophthalmic | | | | | |
| type) | | | | | |
| Hemophagocytic | 18646 | 5551 | PRF1 | 603553 | 1 |
| lymphohistiocytosis, familial, 2 | | | | | |
| Immunodeficiency 26, with or | 19090 | 5591 | PRKDC | 615966 | 1 |
| without neurologic abnormalities | | | | | |
| Dystonia 16 | 23992 | 8575 | PRKRA | 612067 | 1 |
| Hypoparathyroidism, familial | 19226 | 5741 | PTH | 146200 | 1 |
| isolated 1 | | | | | |
| Hyperphenylalaninemia, BH4- | 19286 | 5805 | PTS | 261640 | 1 |
| deficient, A | | | | | |
| Myopathy, lactic acidosis, and | 56361 | 80324 | PUS1 | 600462 | 1 |
| sideroblastic anemia 1 | | | | | |
| Intellectual developmental disorder | 78697 | 54517 | PUS7 | 618342 | 1 |
| with abnormal behavior, | | | | | |
| microcephaly, and short stature | | | | | |
| Leukodystrophy, hypomyelinating, | 69051 | 29920 | PYCR2 | 616420 | 1 |
| 10 | | | | | |
| Combined oxidative | 76563 | 55278 | QRSL1 | 618835 | 1 |
| phosphorylation deficiency 40 | | | | | |
| Carpenter syndrome | 19335 | 51715 | RAB23 | 201000 | 1 |
| Immunodeficiency 73C with | 19354 | 5880 | RAC2 | 618987 | 1 |
| defective neutrophil chemotaxis | | | | | |
| and hypogammaglobulinemia | | | | | |
| Leber congenital amaurosis 12 | 74023 | 343035 | RD3 | 610612 | 1 |
| Deafness, autosomal recessive 24 | 19684 | 5962 | RDX | 611022 | 1 |
| Aicardi-Goutieres syndrome 3 | 68209 | 84153 | RNASEH2C | 610329 | 1 |
| RIDDLE syndrome | 70238 | 165918 | RNF168 | 611943 | 1 |
| Robinow syndrome, autosomal | 26564 | 4920 | ROR2 | 268310 | 1 |
| recessive | | | | | |
| Ribose 5-phosphate isomerase | 19895 | 22934 | RPIA | 608611 | 1 |
| deficiency | | | | | |
| Mitochondrial complex II | 67680 | 6390 | SDHB | 619224 | 1 |
| deficiency, nuclear type 4 | | | | | |
| Spinocerebellar ataxia, autosomal | 269254 | 23064 | SETX | 606002 | 1 |
| recessive, with axonal neuropathy | | | | | |
| 2 | | | | | |
| Neurodevelopmental disorder with | 108037 | 6472 | SHMT2 | 619121 | 1 |
| cardiomyopathy, spasticity, and | | | | | |
| brain abnormalities | | | | | |
| Albinism, oculocutaneous, type VI | 317750 | 283652 | SLC24A5 | 113750 | 1 |
| Skin/hair/eye pigmentation 4, | 317750 | 283652 | SLC24A5 | 113750 | 1 |
| fair/dark skin] | | | | | |
| Citrullinemia, adult-onset type II | 50799 | 10165 | SLC25A13 | 603471 | 1 |
| Citrullinemia, type II, neonatal- | 50799 | 10165 | SLC25A13 | 605814 | 1 |
| onset | | | | | |
| Congenital disorder of | 67547 | 64116 | SLC39A8 | 616721 | 1 |
| glycosylation, type IIn | | | | | |
| Parkinsonism-dystonia, infantile, 1 | 13162 | 6531 | SLC6A3 | 613135 | 1 |
| Lichtenstein-Knorr syndrome | 20544 | 6548 | SLC9A1 | 616291 | 1 |
| Heart and brain malformation | 71997 | 56006 | SMG9 | 616920 | 1 |
| syndrome | | | | | |
| Dentin dysplasia, type I, with | 64074 | 64094 | SMOC2 | 125400 | 1 |
| microdontia and misshapen teeth | | | | | |
| Osteopetrosis, autosomal recessive | 71982 | 29887 | SNX10 | 615085 | 1 |
| 8 | | | | | |
| Ovarian dysgenesis 9 | 224008 | 23514 | SPIDR | 619665 | 1 |
| Deafness, autosomal recessive 115 | 216892 | 124976 | SPNS2 | 618457 | 1 |
| Dystonia, dopa-responsive, due to | 20751 | 6697 | SPR | 612716 | 1 |
| sepiapterin reductase deficiency | | | | | |
| Pyropoikilocytosis | 20739 | 6708 | SPTA1 | 266140 | 1 |
| Spherocytosis, type 3 | 20739 | 6708 | SPTA1 | 270970 | 1 |
| Immunodeficiency 31B, | 20846 | 6772 | STAT1 | 613796 | 1 |
| mycobacterial and viral infections, | | | | | |
| autosomal recessive | | | | | |
| Hemophagocytic | 74732 | 8676 | STX11 | 603552 | 1 |
| lymphohistiocytosis, familial, 4 | | | | | |
| Intellectual developmental | 319944 | 6873 | TAF2 | 615599 | 1 |
| disorder, autosomal recessive 40 | | | | | |
| Hypertryptophanemia] | 56720 | 6999 | TDO2 | 600627 | 1 |
| Spinocerebellar ataxia, autosomal | 104884 | 55775 | TDP1 | 607250 | 1 |
| recessive, with axonal neuropathy | | | | | |
| 1 | | | | | |
| Osteogenesis imperfecta, type | 212943 | 55603 | TENT5A | 617952 | 1 |
| XVIII | | | | | |
| Catel-Manzke syndrome | 76355 | 23483 | TGDS | 616145 | 1 |
| Segawa syndrome, recessive | 21823 | 7054 | TH | 605407 | 1 |
| Spinocerebellar ataxia, autosomal | 66628 | 54974 | THG1L | 618800 | 1 |
| recessive 28 | | | | | |
| Paget disease of bone 5, juvenile- | 18383 | 4982 | TNFRSF11B | 239000 | 1 |
| onset | | | | | |
| Mosaic variegated aneuploidy | 69716 | 9319 | TRIP13 | 617598 | 1 |
| syndrome 3 | | | | | |
| Oocyte maturation defect 9 | 69716 | 9319 | TRIP13 | 619011 | 1 |
| Intellectual developmental | 212528 | 55621 | TRMT1 | 618302 | 1 |
| disorder, autosomal recessive 68 | | | | | |
| Immunodeficiency 35 | 54721 | 7297 | TYK2 | 611521 | 1 |
| Beta-ureidopropionase deficiency | 103149 | 51733 | UPB1 | 613161 | 1 |
| Leber congenital amaurosis 19 | 77593 | 85015 | USP45 | 618513 | 1 |
| Combined oxidative | 68915 | 57176 | VARS2 | 615917 | 1 |
| phosphorylation deficiency 20 | | | | | |
| Galloway-Mowat syndrome 6 | 57773 | 10785 | WDR4 | 618347 | 1 |
| Microcephaly, growth deficiency, | 57773 | 10785 | WDR4 | 618346 | 1 |
| seizures, and brain malformations | | | | | |
| Osteogenesis imperfecta, type XV | 22408 | 7471 | WNT1 | 615220 | 1 |
| Split-hand/foot malformation 6 | 22410 | 7480 | WNT10B | 225300 | 1 |
| Dyskeratosis congenita, autosomal | 216853 | 55135 | WRAP53 | 613988 | 1 |
| recessive 3 | | | | | |
| Xanthinuria, type I | 22436 | 7498 | XDH | 278300 | 1 |
| Spastic paraplegia 15, autosomal | 211978 | 23503 | ZFYVE26 | 270700 | 1 |
| recessive | | | | | |

### XVII. Polynucleotides and Methods of Making

A polynucleotide includes DNA and RNA. Provided herein is any polynucleotide disclosed herein, for example, encoding an anti-TfR:Payload fusion protein, e.g., anti-TfR scFv:GAA or anti-TfR Fab:GAA (e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263), optionally, which is operably linked to a promoter or other expression control sequence. Polypeptides encoded by such polynucleotides are also provided herein.

Nucleotide sequences of HCVRs and LCVRs of anti-hTfR:Payload fusion proteins set forth herein are summarized below in Table P. Polynucleotides encoding an anti-hTfR:Payload fusion protein that includes one or more of the HCVRs and/or LCVRs set forth in Table P are provided herein.

TABLE P
Nucleotide Sequences encoding Domains in Antibodies,
Antigen-binding Fragments (e.g., Fabs) or scFv
Molecules in Fusion Proteins (an anti-hTfR).

| Anti-hTfR Molecule | HCVR | LCVR |
|---|---|---|
| 31874B | 1 | 6 |
| 31863B | 11 | 16 |
| 69348 | 21 | 26 |
| 69340 | 31 | 36 |
| 69331 | 41 | 46 |
| 69332 | 51 | 56 |
| 69326 | 61 | 66 |
| 69329 | 71 | 76 |
| 69323 | 81 | 86 |
| 69305 | 91 | 96 |
| 69307 | 101 | 106 |
| 12795B | 111 | 116 |
| 12798B | 121 | 126 |
| 12799B | 131 | 136 |
| 12801B | 141 | 146 |
| 12802B | 151 | 156 |
| 12808B | 161 | 166 |
| 12812B | 171 | 176 |
| 12816B | 181 | 186 |
| 12833B | 191 | 196 |
| 12834B | 201 | 206 |
| 12835B | 211 | 216 |
| 12847B | 221 | 226 |
| 12848B | 231 | 236 |
| 12843B | 241 | 246 |
| 12844B | 251 | 256 |
| 12845B | 261 | 266 |
| 12839B | 271 | 276 |
| 12841B | 281 | 286 |
| 12850B | 291 | 296 |
| 69261 | 301 | 306 |
| 69263 | 311 | 316 |

For example, provided herein is a polynucleotide encoding an anti-hTfR:Payload fusion protein that includes:
a polynucleotide encoding a HCVR that comprises the nucleotide sequence set forth in SEQ TD NO: 1, and a LCVR that comprises the nucleotide sequence set forth in SEQ TD NO: 6;
a polynucleotide encoding a HCVR that comprises the nucleotide sequence set forth in SEQ TD NO: 11, and a LCVR that comprises the nucleotide sequence set forth in SEQ TD NO: 16;
a polynucleotide encoding a HCVR that comprises the nucleotide sequence set forth in SEQ ID NO: 21, and a LCVR that comprises the nucleotide sequence set forth in SEQ ID NO: 26;
a polynucleotide encoding a HCVR that comprises the nucleotide sequence set forth in SEQ ID NO: 31, and a LCVR that comprises the nucleotide sequence set forth in SEQ ID NO: 36;
a polynucleotide encoding a HCVR that comprises the nucleotide sequence set forth in SEQ ID NO: 41, and a LCVR that comprises the nucleotide sequence set forth in SEQ ID NO: 46;
a polynucleotide encoding a HCVR that comprises the nucleotide sequence set forth in SEQ ID NO: 51, and a LCVR that comprises the nucleotide sequence set forth in SEQ ID NO: 56;
a polynucleotide encoding a HCVR that comprises the nucleotide sequence set forth in SEQ ID NO: 61, and a LCVR that comprises the nucleotide sequence set forth in SEQ ID NO: 66;
a polynucleotide encoding a HCVR that comprises the nucleotide sequence set forth in SEQ ID NO: 71, and a LCVR that comprises the nucleotide sequence set forth in SEQ ID NO: 76;
a polynucleotide encoding a HCVR that comprises the nucleotide sequence set forth in SEQ ID NO: 81, and a LCVR that comprises the nucleotide sequence set forth in SEQ ID NO: 86;
a polynucleotide encoding a HCVR that comprises the nucleotide sequence set forth in SEQ ID NO: 91, and a LCVR that comprises the nucleotide sequence set forth in SEQ ID NO: 96;
a polynucleotide encoding a HCVR that comprises the nucleotide sequence set forth in SEQ ID NO: 101, and a LCVR that comprises the nucleotide sequence set forth in SEQ ID NO: 106;
a polynucleotide encoding a HCVR that comprises the nucleotide sequence set forth in SEQ ID NO: 111, and a LCVR that comprises the nucleotide sequence set forth in SEQ ID NO: 116;
a polynucleotide encoding a HCVR that comprises the nucleotide sequence set forth in SEQ ID NO: 121, and a LCVR that comprises the nucleotide sequence set forth in SEQ ID NO: 126;
a polynucleotide encoding a HCVR that comprises the nucleotide sequence set forth in SEQ ID NO: 131, and a LCVR that comprises the nucleotide sequence set forth in SEQ ID NO: 136;
a polynucleotide encoding a HCVR that comprises the nucleotide sequence set forth in SEQ ID NO: 141, and a LCVR that comprises the nucleotide sequence set forth in SEQ ID NO: 146;
a polynucleotide encoding a HCVR that comprises the nucleotide sequence set forth in SEQ ID NO: 151, and a LCVR that comprises the nucleotide sequence set forth in SEQ ID NO: 156;
a polynucleotide encoding a HCVR that comprises the nucleotide sequence set forth in SEQ ID NO: 161, and a LCVR that comprises the nucleotide sequence set forth in SEQ ID NO: 166;
a polynucleotide encoding a HCVR that comprises the nucleotide sequence set forth in SEQ ID NO: 171, and a LCVR that comprises the nucleotide sequence set forth in SEQ ID NO: 176;
a polynucleotide encoding a HCVR that comprises the nucleotide sequence set forth in SEQ ID NO: 181, and a LCVR that comprises the nucleotide sequence set forth in SEQ ID NO: 186;
a polynucleotide encoding a HCVR that comprises the nucleotide sequence set forth in SEQ ID NO: 191, and a LCVR that comprises the nucleotide sequence set forth in SEQ ID NO: 196;
a polynucleotide encoding a HCVR that comprises the nucleotide sequence set forth in SEQ ID NO: 201, and a LCVR that comprises the nucleotide sequence set forth in SEQ ID NO: 206;
a polynucleotide encoding a HCVR that comprises the nucleotide sequence set forth in SEQ ID NO: 211, and a LCVR that comprises the nucleotide sequence set forth in SEQ ID NO: 216;
a polynucleotide encoding a HCVR that comprises the nucleotide sequence set forth in SEQ ID NO: 221, and a LCVR that comprises the nucleotide sequence set forth in SEQ ID NO: 226;
a polynucleotide encoding a HCVR that comprises the nucleotide sequence set forth in SEQ ID NO: 231, and a LCVR that comprises the nucleotide sequence set forth in SEQ ID NO: 236;
a polynucleotide encoding a HCVR that comprises the nucleotide sequence set forth in SEQ ID NO: 241, and a LCVR that comprises the nucleotide sequence set forth in SEQ ID NO: 246;
a polynucleotide encoding a HCVR that comprises the nucleotide sequence set forth in SEQ ID NO: 251, and a LCVR that comprises the nucleotide sequence set forth in SEQ ID NO: 256;
a polynucleotide encoding a HCVR that comprises the nucleotide sequence set forth in SEQ ID NO: 261, and a LCVR that comprises the nucleotide sequence set forth in SEQ ID NO: 266;
a polynucleotide encoding a HCVR that comprises the nucleotide sequence set forth in SEQ ID NO: 271, and a LCVR that comprises the nucleotide sequence set forth in SEQ ID NO: 276;
a polynucleotide encoding a HCVR that comprises the nucleotide sequence set forth in SEQ ID NO: 281, and a LCVR that comprises the nucleotide sequence set forth in SEQ ID NO: 286;
a polynucleotide encoding a HCVR that comprises the nucleotide sequence set forth in SEQ ID NO: 291, and a LCVR that comprises the nucleotide sequence set forth in SEQ ID NO: 296;
a polynucleotide encoding a HCVR that comprises the nucleotide sequence set forth in SEQ ID NO: 301, and a LCVR that comprises the nucleotide sequence set forth in SEQ ID NO: 306; or
a polynucleotide encoding a HCVR that comprises the nucleotide sequence set forth in SEQ ID NO: 311, and a LCVR that comprises the nucleotide sequence set forth in SEQ ID NO: 316;
   for example, fused to a polynucleotide encoding a payload,
   wherein the HCVR and LCVR are in either order.

In general, a “promoter” or “promoter sequence” is a DNA regulatory region capable of binding an RNA polymerase in a cell (e.g., directly or through other promoter-bound proteins or substances) and initiating transcription of a coding sequence. A promoter may be operably linked to other expression control sequences, including enhancer and repressor sequences and/or with a polynucleotide provided herein. Promoters which may be used to control gene expression include, but are not limited to, cytomegalovirus (CMV) promoter (U.S. Pat. Nos. 5,385,839 and 5,168,062), the SV40 early promoter region (Benoist, et al., (1981) Nature 290:304-310), the promoter contained in the 3′ long terminal repeat of Rous sarcoma virus (Yamamoto, et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster, et al., (1982) Nature 296:39-42); prokaryotic expression vectors such as the beta-lactamase promoter (VIIIa-Komaroff, et al., (1978) Proc. Natl. Acad. Sci. USA 75:3727-3731), or the tac promoter (DeBoer, et al., (1983) Proc. Natl. Acad. Sci. USA 80:21-25); see also “Useful proteins from recombinant bacteria” in Scientific American (1980) 242:74-94; and promoter elements from yeast or other fungi such as the Ga4 promoter, the ADC (alcohol dehydrogenase) promoter, PGK (phosphoglycerol kinase) promoter or the alkaline phosphatase promoter.

A polynucleotide encoding a polypeptide is “operably linked” to a promoter or other expression control sequence when, in a cell or other expression system, the sequence directs RNA polymerase mediated transcription of the coding sequence into RNA, preferably mRNA, which then may be RNA spliced (if it contains introns) and, optionally, translated into a protein encoded by the coding sequence.

Provided herein are polynucleotides encoding polypeptide chains which are variants of those whose nucleotide sequence is specifically set forth herein. A “variant” of a polynucleotide refers to a polynucleotide comprising a nucleotide sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical to a referenced nucleotide sequence that is set forth herein (see e.g., the nucleotide sequences of Table P); when the comparison is performed by a BLAST algorithm wherein the parameters of the algorithm are selected to give the largest match between the respective sequences over the entire length of the respective reference sequences (e.g., expect threshold: 10; word size: 28; max matches in a query range: 0; match/mismatch scores: 1, −2; gap costs: linear). In an embodiment, a variant of a nucleotide sequence specifically set forth herein comprises one or more (e.g., 1, 2, 3, 4, 5, 6, 7, 8, 9, 10, 11 or 12) point mutations, insertions (e.g., in frame insertions) or deletions (e.g., in frame deletions) of one or more nucleotides. Such mutations may, in an embodiment, be missense or nonsense mutations.

Eukaryotic and prokaryotic host cells, including mammalian cells, may be used as hosts for expression of an anti-TfR:Payload fusion protein. Such host cells are well known in the art and many are available from the American Type Culture Collection (ATCC). These host cells include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Mammalian host cells include human, mouse, rat, dog, monkey, pig, goat, bovine, horse and hamster cells. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia, Pichiapastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens and Neurospora crassa. Provided herein is an isolated host cell (e.g., a CHO cell or any type of host cell set forth above) comprising an anti-TfR:Payload such as 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; and 69263.

In addition, also provided herein is a complex comprising an anti-TfR:Payload, as discussed herein complexed with a transferrin receptor polypeptide or an antigenic fragment thereof or fusion thereof and/or with a secondary antibody or antigen-binding fragment thereof (e.g., detectably labeled secondary antibody) that binds specifically to the anti-TfR:Payload. In an embodiment, the complex is in vitro (e.g., is immobilized to a solid substrate) or is in the body of a subject.

Recombinant anti-TfR:Payload fusion proteins disclosed herein may also be produced in an E. coli/T7 expression system. In this embodiment, polynucleotides encoding the anti-TfR:Payload fusion proteins disclosed herein (e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; and 69263) may be inserted into a pET-based plasmid and expressed in the E. coli/T7 system. For example, provided herein are methods for expressing anti-TfR:Payload fusion proteins in a host cell (e.g., bacterial host cell such as E. coli such as BL21 or BL21DE3) comprising expressing T7 RNA polymerase in the cell which also includes a polynucleotide encoding the anti-TfR:Payload fusion protein that is operably linked to a T7 promoter. For example, in an embodiment, a bacterial host cell, such as an E. coli, includes a polynucleotide encoding the T7 RNA polymerase gene operably linked to a lac promoter and expression of the polymerase and the chain is induced by incubation of the host cell with IPTG (isopropyl-beta-D-thiogalactopyranoside). See U.S. Pat. Nos. 4,952,496 and 5,693,489 or Studier & Moffatt, Use of bacteriophage T7 RNA polymerase to direct selective high-level expression of cloned genes, J. Mol. Biol. 1986 May 5; 189(1): 113-30.

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, polynucleotides may be introduced into mammalian cells by viral vectors. Methods of transforming cells are well known in the art. See, for example, U.S. Pat. Nos. 4,399,216; 4,912,040; 4,740,461 and 4,959,455. Thus, provided herein are recombinant methods for making an anti-TfR:Payload fusion protein disclosed herein comprising (i) introducing, into a host cell, one or more polynucleotides encoding the anti-TfR:Payload, for example, wherein the polynucleotide is in a vector; and/or integrates into the host cell chromosome and/or is operably linked to a promoter; (ii) culturing the host cell (e.g., CHO or Pichia or Pichia pastoris) under conditions favorable to expression of the polynucleotide and, (iii) optionally, isolating the anti-TfR:Payload fusion protein from the host cell and/or medium in which the host cell is grown. Also provided are anti-TfR:Payload fusion protein which are the product of the production methods set forth herein, and, optionally, the purification methods set forth herein.

In an embodiment, a method for making an anti-TfR:Payload fusion protein, includes a method of purifying the anti-TfR:Payload fusion protein, e.g., by column chromatography, precipitation and/or filtration. As discussed, the product of such a method are also provided herein.

In one aspect, provided is a method of producing an anti-TfR:Payload fusion protein in a cell. In one embodiment, the anti-TfR:Payload fusion protein is produced by administering to the cell a gene therapy vector comprising a polynucleotide encoding the anti-TfR:Payload fusion protein. In one embodiment, the polynucleotide subsequently integrates at a genomic locus (e.g., in the liver) and the encoded fusion protein is produced (i.e., the polynucleotide is transcribed and translated). In another embodiment, the polynucleotide is transcribed episomally (e.g., in the liver) and the encoded fusion protein is produced.

In some embodiments, the methods further comprise administering a nuclease agent or one or more polynucleotides encoding the nuclease agent to the cell, wherein the nuclease agent cleaves the genomic locus, and the polynucleotide encoding the anti-TfR:Payload fusion protein is integrated into the genomic locus. Suitable nuclease agents include, for example, Clustered Regularly Interspersed Short Palindromic Repeats (CRISPR)/CRISPR-associated (Cas) systems, zinc finger nuclease (ZFN) systems, or Transcription Activator-Like Effector Nuclease (TALEN) systems.

In some embodiments, the polynucleotide encoding the anti-TfR:Payload fusion protein can comprise flanking homology arms for integration into the genomic locus by homology-directed repair. In other embodiments, the polynucleotide does not include homology arms, such as for integration into the genomic locus by non-homologous end joining.

In some embodiments, the gene therapy vector is one that is commonly used in cell transfection, such as an adeno-associated virus (AAV) vector. In some embodiments, the gene therapy vector is selected from the group consisting of a viral vector, optionally wherein the viral vector is a natural virus, an engineered virus, or a chimeric virus, and a naked polynucleotide comprising a polynucleotide described herein, a polynucleotide complex, optionally wherein the polynucleotide complex is a lipid nanoparticle comprising the polynucleotide and lipids, and any combination thereof. In some embodiments, the gene therapy vector is a viral vector, optionally selected from the group consisting of a retrovirus, adenovirus, a herpes simplex virus, a pox virus, a vaccinia virus, a lentivirus, or an adeno-associated virus. In some embodiments, the gene therapy vector is AAV9, Anc80, a recombinant AAV8 (e.g., an AAV2/8 chimera) and/or an AAV pseudotyped to a specific tissue, e.g., the liver or neuronal tissue.

In some embodiments, the genomic locus into which the polynucleotide encoding the anti-TfR:Payload fusion protein is integrated is a “safe harbor locus.” In one embodiment, a “safe harbor locus” enables high expression of the anti-TfR:Payload fusion protein, while not interfering with the expression of essential genes or promoting the expression of oncogenes or other deleterious genes. In one embodiment, the genomic locus is at or proximal to the liver-expressed albumin (Alb) locus, a EESYR locus, a SARS locus, position 188,083,272 of human chromosome 1 or its non-human mammalian orthologue, position 3,046,320 of human chromosome 10 or its non-human mammalian orthologue, position 67,328,980 of human chromosome 17 or its non-human mammalian orthologue, an adeno-associated virus site 1 (AAVS1) on chromosome, a naturally occurring site of integration of AAV virus on human chromosome 19 or its non-human mammalian orthologue, a chemokine receptor 5 (CCR5) gene, a chemokine receptor gene encoding an HIV-1 coreceptor, or a mouse Rosa26 locus or its non-murine mammalian orthologue. In one embodiment, the genomic locus is an adeno-associated virus site. In one embodiment, the genomic locus for integration is selected according to the method of Papapetrou and Schambach, J. Molecular Therapy, vol. 24 (4):678-684, April 2016, which is herein incorporated by reference for the stepwise selection of a safe harbor genomic locus for gene therapy vector integration; see also Barzel et al. Nature, vol. 517:360-364, incorporated herein by reference in its entirety, for the promoterless gene targeting into the liver-expressed albumin (Alb) locus.

In some embodiments, the polynucleotide, e.g., DNA, also contains a promoter operably linked to the anti-TfR:Payload fusion protein encoding sequence. In a specific embodiment, the promoter is a tissue-specific promotor that drives gene expression in a particular tissue. In one embodiment, the tissue specific promoter is a liver-specific enhancer/promoter derived from serpinal and/or is a TTR promoter. In other embodiments, the promoter is a CMV promoter. In other embodiments, the promoter is a ubiquitin C promoter.

In one embodiment, the cell is a mammalian cell, such as a human cell or a mouse cell. In one embodiment, the cell is a liver cell, such as a mammalian liver cell, a human liver cell, or a mouse liver cell. In one embodiment, the cell is ex vivo. In another embodiment, the cell is in vivo, and the gene therapy vector containing the polynucleotide encoding the anti-TfR:Payload fusion protein is administered to a subject (e.g., a human or non-human subject).

In one embodiment, the polynucleotide encoding the anti-TfR:Payload fusion protein is used to treat a subject in need of enzyme replacement therapy (e.g., in a method of delivering a therapeutic protein to the central nervous system (CNS) of a subject), comprising administering to the subject a gene therapy vector comprising a polynucleotide encoding the anti-TfR:Payload fusion protein (e.g., via a liver-targeted delivery method sufficient to provide a therapeutically effective amount of the anti-TfR:Payload fusion protein in the CNS). In some embodiments, the subject is an animal. In some embodiments, the subject is a human. In one embodiment, the polynucleotide subsequently integrates at a genomic locus in the liver and the encoded fusion protein is produced. In another embodiment, the polynucleotide is transcribed episomally in the liver and the encoded fusion protein is produced.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### Brief Description of the Sequences

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

### EXAMPLES

### Example 1. Generation, Selection and Characterization of Immunoglobulin Molecules

Anti-human transferrin receptor (hTfR) antibodies were generated and screened for the ability to bind hTfR and for lack of strong blocking of human transferrin-hTfR binding.

Anti-hTfR Generation. VelocImmune mice were immunized with a recombinant protein comprising human transferrin receptor extracellular domain fused at N-terminus to a 6-His tag (referred to as human 6×His-TfR) as immunogen via subcutaneous footpad injection with Alum:CpG adjuvant. Mice bleeds were collected prior to the initial immunization injection and post-boost injections, and the immune sera were subjected to antibody titer determination using a human TfR specific enzyme-linked immunosorbent assay (ELISA). In this assay serum samples in serial dilutions were added to the immunogen coated plates and plate-bound mouse IgG were detected using an HRP-conjugated anti-mouse IgG antibody. Titer of a tested serum sample is defined as the extrapolated dilution factor of the sample that produces a binding signal two times of the signal of the buffer alone control sample. The mice with optimal anti-TfR antibody titers were selected and subjected to a final boost 3-5 days prior to euthanasia and splenocytes from these mice were harvested and subject to antibody isolation using B cell sorting technology (BST).

TfR specific antibodies of isolated antibodies were isolated and characterized. Two hundred and fourteen TfR-binding antibodies were cloned into single chain fragment variables (scFvs) in complementary orientations with either the variable heavy chain followed by the variable light chain (V_{H}-V_{K}), or the variable light chain followed by the variable heavy chain (V_{K}-VH), and as fragment antigen-binding regions (Fabs). Conditioned media of CHO cell culture containing the scFvs or Fabs were tested for the ability to bind hTfR proteins and hTfR-expressing cells.

### Example 2. Binding Kinetics of 32 Anti-hTfR Primary Supernatants from CHO

Biacore binding kinetics assays were conducted for the interaction of 32 anti-human TfR IgG1 monoclonal antibodies from CHO supernatants with TfR reagents at 25° C.

TABLE 2-1
Monoclonal Antibody Clones Tested

| mAb# | AbID# | Source |
|---|---|---|
| 1 | 12795B | primary supernatant |
| 2 | 12798B | primary supernatant |
| 3 | 12799B | primary supernatant |
| 4 | 12801B | primary supernatant |
| 5 | 12802B | primary supernatant |
| 6 | 12808B | primary supernatant |
| 7 | 12812B | primary supernatant |
| 8 | 12834B | primary supernatant |
| 9 | 12835B | primary supernatant |
| 10 | 12839B | primary supernatant |
| 11 | 12841B | primary supernatant |
| 12 | 12843B | primary supernatant |
| 13 | 12844B | primary supernatant |
| 14 | 12845B | primary supernatant |
| 15 | 12847B | primary supernatant |
| 16 | 12848B | primary supernatant |
| 17 | 12850B | primary supernatant |
| 18 | 31863B | primary supernatant |
| 19 | 31874B | primary supernatant |
| 20 | 12816B | primary supernatant |
| 21 | 12833B | primary supernatant |
| 22 | 69261 | primary supernatant |
| 23 | 69263 | primary supernatant |
| 24 | 69305 | primary supernatant |
| 25 | 69307 | primary supernatant |
| 26 | 69323 | primary supernatant |
| 27 | 69326 | primary supernatant |
| 28 | 69329 | primary supernatant |
| 29 | 69331 | primary supernatant |
| 30 | 69332 | primary supernatant |
| 31 | 69340 | primary supernatant |
| 32 | 69348 | primary supernatant |
| 33 | REGN1945 | |

Reagents Used:
REGN2431 (hmm.hTfRC; 79210 g/mol molecular weight), having the amino acid sequence:

(SEQ ID NO: 460)
HHHHHHEQKLISEEDLGGEQKLISEEDLCKGVEPKTECERLAGTESPVR
EEPGEDFPAARRLYWDDLKRKLSEKLDSTDFTGTIKLLNENSYVPREAG
SQKDENLALYVENQFREFKLSKVWRDQHFVKIQVKDSAQNSVIIVDKNG
RLVYLVENPGGYVAYSKAATVTGKLVHANFGTKKDFEDLYTPVNGSIVI
VRAGKITFAEKVANAESLNAIGVLIYMDQTKFPIVNAELSFFGHAHLGT
GDPYTPGFPSFNHTQFPPSRSSGLPNIPVQTISRAAAEKLFGNMEGDCP
SDWKTDSTCRMVTSESKNVKLTVSNVLKEIKILNIFGVIKGFVEPDHYV
VVGAQRDAWGPGAAKSGVGTALLLKLAQMFSDMVLKDGFQPSRSIIFAS
WSAGDEGSVGATEWLEGYLSSLHLKAFTYINLDKAVLGTSNFKVSASPL
LYTLIEKTMQNVKHPVTGQFLYQDSNWASKVEKLTLDNAAFPFLAYSGI
PAVSFCFCEDTDYPYLGTTMDTYKELIERIPELNKVARAAAEVAGQFVI
KLTHDVELNLDYERYNSQLLSFVRDLNQYRADIKEMGLSLQWLYSARGD
FFRATSRLTTDFGNAEKTDRFVMKKLNDRVMRVEYHELSPYVSPKESPF
RHVFWGSGSHTLPALLENLKLRKQNNGAFNETLERNQLALATWTIQGAA
NALSGDVWDIDNEF.

REGN2054 (mf TFRC ecto-mmh; 78500 g/mol molecular weight):
   monomeric monkey (cyno) Tfrc ectodomain (amino acids C89-F760, Accession #: XP_045243212.1) with a c-terminal myc-myc-hexahistidine tag containing a GG linker (underlined) between the 2 myc epitope sequences (EQKLISEEDLGGEQKLISEEDLHHHHHH (SEQ ID NO: 461)).

Equilibrium dissociation constants (K_{D}) for the interaction of anti-TfR monoclonal antibodies with human and fascicularis monkey TfR ecto domain recombinant proteins were determined using a real-time surface plasmon resonance (SPR) based Biacore S200 biosensor. All binding studies were performed in 10 mM HEPES, 150 mM NaCl, 3 mM EDTA, and 0.05% v/v surfactant Tween-20, pH 7.4 (HBS-EP) running buffer at 37° C. The Biacore CM5 sensor surface was first derivatized by amine coupling with a monoclonal mouse anti-human Fc antibody (REGN2567) followed by a step to capture anti-TfR monoclonal antibodies in CHO conditioned media. Human TfR extracellular domain expressed with a C-terminal myc-myc-hexahistidine tag (hTFR-mmh; REGN2431) or monkey TfR extracellular domain expressed with a C-terminal myc-myc-hexahistidine tag (mfTFR-mmh; REGN2054) at concentrations of 100 nM in HBS-EP running buffer were injected at a flow rate of 50 μL/min for 2 minutes. The dissociation of TfR bound to anti-TfR monoclonal antibodies was monitored for 3 minutes in HBS-EP running buffer. At the end of each cycle, the anti-TfR monoclonal antibodies capture surface was regenerated using a 12-sec injection of 20 mM H₃PO₄. The association rate (ka) and dissociation rate (kd) were determined by fitting the real-time binding sensorgrams to a 1:1 binding model with mass transport limitation using Scrubber 2.0c software. The dissociation equilibrium constant (K_{D}) and dissociative half-life (t½) were calculated from the kinetic rate constants as:

$${K}_{D} ( M ) = \frac{k d}{k a} , and t 1 / 2 ( min ) = \frac{ln ( 2 )}{60 * k d}$$

The equilibrium binding constant and the kinetic binding constants are summarized in Tables 2-2 and Table 2-3 for human TfR and monkey TfR, respectively. At 25° C., anti-TfR monoclonal antibodies bound to hTfR-mmh with K_{D} values ranging from 65.6 pM to 41 nM, as shown in Table 2-2. Anti-TfR monoclonal antibodies bound to mfTfR-mmh with K_{D} values ranging from 1.16 nM to 20.5 nM, as shown in Table 2-3.

Results are set forth below.

TABLE 2-2
Equilibrium and kinetic binding parameters for the interaction of
hTFR-mmh with anti-TfR monoclonal antibodies (bivalent IgG)
at 25° C.

| | mAb | 100 | | | | |
|---|---|---|---|---|---|---|
| | Cap- | nM | | | | |
| | ture | Ag | | | | |
| | Level | Bound | kₐ | k_{d} | K_{D} | t½ |
| Molecule | (RU) | (RU) | (1/Ms) | (1/s) | (M) | (min) |
| 12795B | 3344 | 883 | 1.22E+05 | <1.00E−05 | 8.23E−11 | >1155^{#} |
| 12798B | 4552 | 1151 | 1.63E+05 | 6.33E−05 | 3.87E−10 | 182.6 |
| 12799B | 2388 | 699 | 7.76E+04 | 6.62E−05 | 8.51E−10 | 174.5 |
| 12801B | 4720 | 598 | 8.19E+04 | 4.19E−05 | 5.13E−10 | 276.0 |
| 12802B | 2828 | 903 | 1.23E+05 | 2.92E−05 | 2.36E−10 | 395.4 |
| 12808B | 4336 | 964 | 1.19E+05 | 1.07E−04 | 8.94E−10 | 108.5 |
| 12812B | 2222 | 11 | 4.33E+05 | 1.31E−03 | 3.02E−09 | 8.8 |
| 12834B | 3837 | 11 | 6.00E+05 | 4.39E−03 | 7.00E−09 | 2.6 |
| 12835B | 3276 | 1146 | 1.34E+05 | 4.17E−04 | 3.11E−09 | 27.7 |
| 12839B | 5192 | 660 | 1.18E+05 | 5.33E−05 | 4.48E−10 | 216.9 |
| 12841B | 2895 | 1151 | 1.73E+05 | 1.16E−04 | 6.70E−10 | 99.8 |
| 12843B | 3080 | 854 | 9.42E+04 | 1.68E−04 | 1.78E−09 | 68.8 |
| 12844B | 2869 | 946 | 1.31E+05 | 1.22E−04 | 9.35E−10 | 95.0 |
| 12845B | 2911 | 1104 | 1.68E+05 | 1.99E−04 | 1.18E−09 | 57.9 |
| 12847B | 4425 | 895 | 1.05E+05 | 1.71E−04 | 1.62E−09 | 67.6 |
| 12848B | 4530 | 823 | 1.01E+05 | 4.99E−05 | 4.94E−10 | 231.6 |
| 12850B | 2990 | 725 | 8.37E+04 | 1.76E−05 | 2.15E−10 | 656.8 |
| 31863B | 5490 | 1083 | 1.52E+05 | <1.00E−05 | 6.56E−11 | >1155^{#} |
| 31874B | 5594 | 904 | 1.38E+05 | 1.15E−04 | 8.36E−10 | 100.5 |
| 12816B | 3377 | 357 | 5.79E+04 | 3.36E−04 | 5.80E−09 | 34.3 |
| 12833B | 3972 | 409 | 7.20E+04 | 2.52E−04 | 3.51E−09 | 45.9 |
| 69261 | 3546 | 462 | 7.00E+04 | 8.08E−05 | 1.16E−09 | 142.9 |
| 69263 | 180 | 150 | 1.77E+05 | 1.15E−03 | 6.50E−09 | 10.0 |
| 69305 | 2726 | 6 | NB* | NB* | NB* | NB* |
| 69307 | 2349 | 1285 | 1.90E+05 | 4.26E−05 | 2.26E−10 | 271.1 |
| 69323 | 4506 | 465 | 7.34E+04 | 1.80E−04 | 2.45E−09 | 64.1 |
| 69326 | 1709 | 680 | 1.15E+05 | 2.56E−04 | 2.22E−09 | 45.1 |
| 69329 | 1573 | 1100 | 1.92E+05 | 1.86E−04 | 9.70E−10 | 62.2 |
| 69331 | 4617 | 87 | 1.14E+05 | 4.66E−03 | 4.10E−08 | 2.5 |
| 69332 | 3915 | 9 | NB* | NB* | NB* | NB* |
| 69340 | 3226 | 999 | 1.44E+05 | 6.23E−05 | 4.29E−10 | 185.4 |
| 69348 | 2848 | 680 | 1.06E+05 | 1.72E−04 | 1.62E−09 | 67.2 |
| REGN1945 | 4011 | 6 | NB | NB | NB | NB |

^{#}indicates no dissociation was observed under the current experimental conditions and the kd value was manually fixed at 1.00E−05 s⁻¹ while fitting the real time binding sensorgrams.
*NB indicates that no binding was observed under the current experimental conditions.

TABLE 2-3
Equilibrium and kinetic binding parameters for the interaction of
mfTfR- mmh with anti-TfR monoclonal antibodies (bivalent IgG) at
25° C.

| | mAb | 100 nM | | | | |
|---|---|---|---|---|---|---|
| | Capture | Ag | | | | |
| | Level | Bound | kₐ | k_{d} | K_{D} | t½ |
| Molecule | (RU) | (RU) | (1/Ms) | (1/s) | (M) | (min) |
| 12795B | 3334 | 364 | 6.10E+04 | 7.13E−05 | 1.16E−09 | 162.0 |
| 12798B | 4542 | 508 | 7.90E+04 | 1.29E−03 | 1.63E−08 | 9.0 |
| 12799B | 2384 | 651 | 8.64E+04 | 1.62E−04 | 1.88E−09 | 71.2 |
| 12801B | 4684 | 159 | 6.40E+04 | 6.53E−04 | 1.02E−08 | 17.7 |
| 12802B | 2819 | 464 | 7.05E+04 | 5.29E−04 | 7.51E−09 | 21.8 |
| 12808B | 4329 | 626 | 7.85E+04 | 4.54E−04 | 5.78E−09 | 25.5 |
| 12812B | 2220 | 1 | NB* | NB* | NB* | NB* |
| 12834B | 3820 | 5 | NB* | NB* | NB* | NB* |
| 12835B | 3262 | 254 | 9.51E+04 | 1.68E−03 | 1.77E−08 | 6.9 |
| 12839B | 5170 | 11 | NB* | NB* | NB* | NB* |
| 12841B | 2888 | 4 | NB* | NB* | NB* | NB* |
| 12843B | 3072 | 399 | 7.90E+04 | 1.18E−03 | 1.50E−08 | 9.8 |
| 12844B | 2857 | 437 | 7.59E+04 | 7.35E−04 | 9.68E−09 | 15.7 |
| 12845B | 2906 | 13 | NB* | NB* | NB* | NB* |
| 12847B | 4420 | 702 | 9.92E+04 | 3.33E−04 | 3.36E−09 | 34.7 |
| 12848B | 4524 | 261 | 5.29E+04 | 9.62E−04 | 1.82E−08 | 12.0 |
| 12850B | 2985 | 87 | 1.01E+05 | 1.57E−03 | 1.55E−08 | 7.3 |
| 31863B | 5480 | 145 | 1.63E+05 | 2.98E−03 | 1.83E−08 | 3.9 |
| 31874B | 5557 | 26 | 6.91E+05 | 9.96E−03 | 1.44E−08 | 1.2 |
| 12816B | 3376 | 315 | 6.52E+04 | 4.85E−04 | 7.44E−09 | 23.8 |
| 12833B | 3966 | 346 | 6.92E+04 | 4.86E−04 | 7.02E−09 | 23.8 |
| 69261 | 3537 | 331 | 6.83E+04 | 4.03E−04 | 5.90E−09 | 28.7 |
| 69263 | 181 | 77 | 1.97E+05 | 3.78E−03 | 1.92E−08 | 3.1 |
| 69305 | 2725 | -7 | NB* | NB* | NB* | NB* |
| 69307 | 2344 | 3 | NB* | NB* | NB | NB* |
| 69323 | 4500 | 24 | 5.58E+05 | 1.06E−02 | 1.90E−08 | 1.1 |
| 69326 | 1707 | 9 | NB* | NB* | NB* | NB* |
| 69329 | 1571 | 8 | NB* | NB* | NB* | NB* |
| 69331 | 4611 | 26 | 4.89E+05 | 1.00E−02 | 2.05E−08 | 1.2 |
| 69332 | 3897 | 1 | NB* | NB* | NB* | NB* |
| 69340 | 3219 | 634 | 8.92E+04 | 7.23E−04 | 8.11E−09 | 16.0 |
| 69348 | 2851 | 433 | 9.60E+04 | 4.61E−04 | 4.80E−09 | 25.1 |
| REGN1945 | 4009 | 4 | NB | NB | NB | NB |

*NB indicates that no binding was observed under the current experimental conditions.

### Example 3. Anti-TfR Antibodies Blocking Human TfRC Monomer Binding to Human Holo-Transferrin by ELISA

An ELISA-based blocking assay was developed to determine the ability of anti-Transferrin Receptor (TfR) antibodies to block the binding of human Transferrin Receptor to human holo-transferrin ligand.

TABLE 3-1
Reagents

| Reagent | Source |
|---|---|
| Human Transferrin polyclonal goat IgG antibody | R&D Systems |
| Human Holo-Transferrin protein | R&D Systems |
| His-myc-myc-hTFRC ecto | Regeneron |
| HRP conjugated c-Myc polyclonal rabbit IgG | Novus Biologicals |
| antibody | |
| 1X PBS | Irvine Scientific |
| 1X PBST (0.05% tween-20 in PBS) | Sigma |
| BSA: albumin solution from bovine serum, 30% | Sigma |
| 3-3′, 5-5′-tetramethylbenzidine (TMB) Substrate A | BD Biosciences |
| 3-3′, 5-5′-tetramethylbenzidine (TMB) Substrate B | BD Biosciences |
| 2N Sulfuric Acid | VWR |
| Reacti-Bind 96-well plates corner notch | ThermoFisher |
| | Scientific |
| VWR 96-Well Deep Well Plates 0.5 ML | VWR |
| Aquamax Plate Washer 2000 | Molecular Devices |
| VICTOR ™ X4 Multilabel Plate Reader | PerkinElmer |

The human Transferrin Receptor recombinant protein, hTFRC, used in the experiment was comprised of hTfR extracellular domain (amino acids C89-F760) expressed with an N-terminal 6-Histidine-myc-myc tag (Hmm.hTfrc (REGN2431): Monomeric human Tfrc ectodomain (amino acids C89-F760, Accession #: NP_001121620.1) with an N-terminal hexahistidine-myc-myc-tag containing a GG linker (underlined) between the 2 myc epitope sequences (HHHHHHEQKLISEEDLGGEQKLISEEDL) (amino acids 1-28 of SEQ ID NO: 460)). The human holo-transferrin ligand protein (holo-Tf) isolated from human plasma was purchased from R&D Systems.

In the blocking assay, the anti-human Transferrin goat IgG polyclonal antibody (anti-hTf pAb) was passively absorbed at a concentration of 2 micrograms/mL in PBS on a 96-well microtiter plate overnight at 4° C. Nonspecific binding sites were subsequently blocked using a 0.5% (w/v) solution of BSA in PBS for 1 hour at room temperature. To the same plate, human holo-Tf was then added at a concentration of 1 micrograms/mL in PBS+0.5% BSA for 2 hours at room temperature. In a separate set of 96-well microtiter plates, solutions of 300 pM Hmm-hTFRC were mixed with TFRC antibody supernatants at 2-fold dilution. After a 1-hour incubation, the mixtures were transferred to the human holo-Tf microtiter plates. After another hour incubation at room temperature, plates were washed, and plate-bound Hmm-hTFRC was detected with horseradish peroxidase (HRP) conjugated rabbit anti-Myc polyclonal antibody. The plates were developed using TMB substrate solution according to the manufacturer's recommended procedure and absorbance at 450 nm was measured on a Victor™ Multilabel Plate Reader.

Percent blocking for the tested anti-TfR antibodies was calculated using the formula below:

$$\% Blocking = 100 - [ \frac{\begin{matrix}( Test antibody - \\ Buffer alone without Hmm ‐ hTFRC )\end{matrix}}{\begin{matrix}( Hmm ‐ hTFRC alone - \\ Buffer alone without Hmm ‐ hTFRC )\end{matrix}} ] ⨯ 100$$

Antibodies that blocked binding of Hmm-hTFRC to human holo-Tf equal or more than 50% were classified as blockers.

The ability of the anti-TfR antibody to block human TFRC binding to human holo-Tf was evaluated using an ELISA-based blocking assay. In this assay, a fixed concentration of Hmm-hTFRC was pre-incubated with anti-TfR antibody containing supernatant before binding to plate immobilized human holo-Tf protein, and the plate-bound Hmm-hTFRC was detected with HRP-conjugated c-Myc specific rabbit polyclonal antibodies.

Thirty-two anti-TfR antibodies cloned into single chain fragment variables (scFvs) in complementary orientations with either the variable heavy chain followed by the variable light chain (V_{H}-VK), or the variable light chain followed by the variable heavy chain (V_{K}-V_{H}) and also as fragment antigen-binding regions (Fabs). All ninety-six anti-TfR antibody supernatants were tested for the ability to block human TFRC binding to human holo-Tf. Ninety-four anti-TfR antibody supernatants showed no or low blocking activity with percentage blocking ranging from 0% to 45%, and these antibodies (Fabs or scFvs formats) were classified as non-blockers (Table 3-2). Only two Fab supernatants had blocking activity greater than 50%, with % blocking values of 64% and 78% respectively.

TABLE 3-2
Summary of Anti-TfR scFv and Fab Supernatants Ability to
Block Human TFRC binding to Immobilized Human Holo-Tf

| | Blocking of Hmm-hTFRC Binding to | | |
|---|---|---|---|
| | Human Holo-Tf, % Blocking | | |
| | Fab | scFv (V_{K} − V_{H}) | scFv (V_{H} − V_{K}) |
| AbPID | Format | Format | Format |
| 12795B | 44 | 23 | 45 |
| 12798B | 10 | 10 | 0 |
| 12799B | 5 | 10 | 26 |
| 12801B | 45 | 27 | 37 |
| 12802B | 15 | 17 | 11 |
| 12808B | 16 | 18 | 19 |
| 12812B | 14 | 12 | 19 |
| 12816B | 14 | 14 | 16 |
| 12833B | 64 | 40 | 22 |
| 12834B | −2 | 11 | −3 |
| 12835B | 78 | 37 | 45 |
| 12839B | 13 | 6 | 23 |
| 12841B | 29 | 1 | 10 |
| 12843B | 10 | 8 | 17 |
| 12844B | 20 | 10 | 12 |
| 12845B | 11 | 3 | 18 |
| 12847B | 3 | 13 | 11 |
| 12848B | 13 | 9 | 19 |
| 12850B | 18 | 8 | 10 |
| 31863B | 24 | 7 | 13 |
| 31874B | 16 | −1 | 14 |
| 69261 | 11 | 16 | 19 |
| 69263 | 14 | 4 | 14 |
| 69305 | 3 | 5 | 3 |
| 69307 | 12 | 12 | 9 |
| 69323 | 12 | 17 | 7 |
| 69326 | −2 | 12 | 18 |
| 69329 | 8 | 19 | 25 |
| 69331 | 9 | 13 | 7 |
| 69332 | 18 | 22 | 6 |
| 69340 | 3 | 13 | 6 |
| 69348 | 40 | 16 | 0 |

### Example 4. Anti-TFRC:GAA Gene Therapy

In this example, the ability of various anti-TFRC molecules to cross the blood-brain barrier and localize to the parenchyma of the brain was evaluated. Delivery of the molecules via episomal AAV liver depot was also evaluated along with rescue of the glycogen storage phenotype in various tissues.

### In Vivo Screening of Anti-hTFRC Scfv by HDD

To further evaluate the anti-human TFRC antibodies that were screened for binding in vitro, in vivo mouse studies in Tfrc^{hum/hum} knock-in mice were performed to evaluate blood-brain-barrier (BBB) crossing. This screen of 31 antibodies revealed 11 that had mature hGAA protein in brain homogenate detected by Western blot.

### GAA Fusions by Hydrodynamic Delivery (HDD)

Human TFRC knock-in mice were injected with DNA plasmids expressing the various anti-hTFRC antibodies in the anti-hTFRC scfv:2×G₄S(SEQ ID NO: 537):hGAA format under the liver-specific mouse TTR promoter. Mice received 50 ug of DNA in 0.9% sterile saline diluted to 10% of the mouse's body weight (0.1 mL/g body weight). Forty-eight hours post-injection, tissues were dissected from mice immediately after sacrifice by CO₂ asphyxiation, snap frozen in liquid nitrogen, and stored at −80° C.

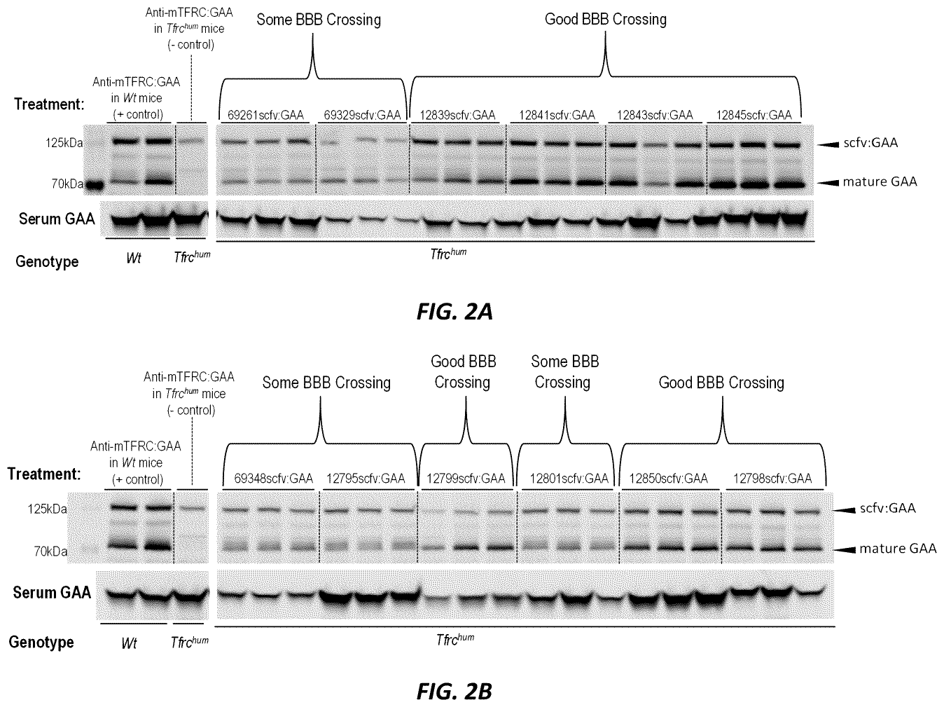

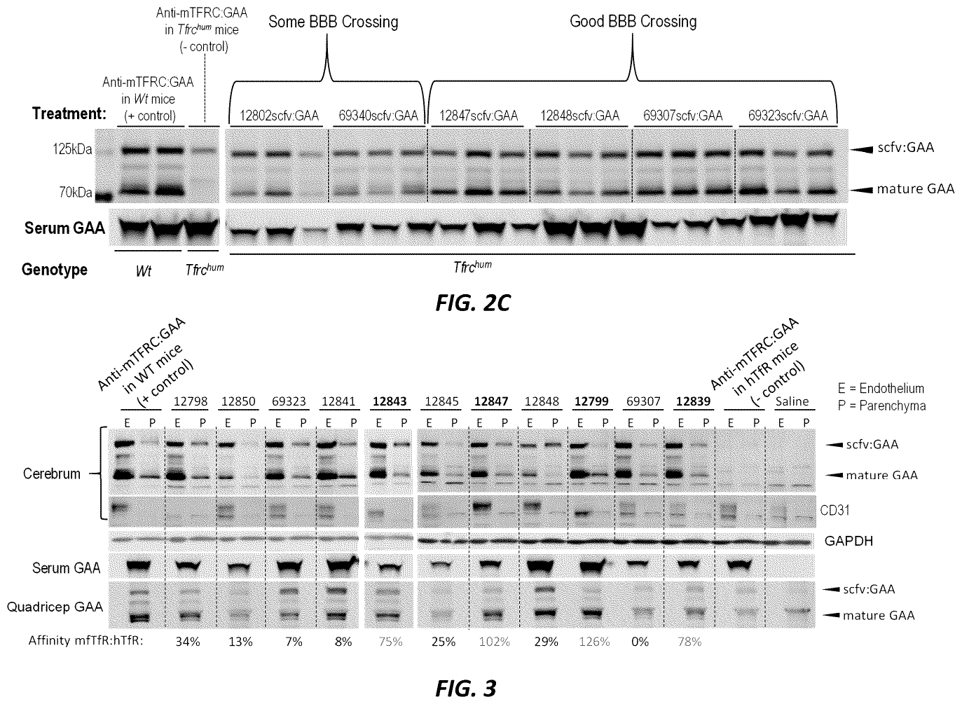

### Western Blot: (FIGS. 2A-2C)

Tissue lysates were prepared by lysis in RIPA buffer with protease inhibitors (1861282, Thermo Fisher, Waltham, MA, USA). Tissue lysates were homogenized with a bead homogenizer (FastPrep5, MP Biomedicals, Santa Ana, CA, USA). Cells or tissue lysates were run on SDS-PAGE gels using the Novex system (LifeTech Thermo, XPO4200BOX, LC2675, LC3675, LC2676). Gels were transferred to low-fluorescence polyvinylidene fluoridev (PVDF) membrane (IPFLO7810, LI-COR, Lincoln, NE, USA) and stained with Revert 700 Total Protein Stain (TPS; 926-11010 LI-COR, Lincoln, NE, USA), followed by blocking with Odyssey blocking buffer (927-500000, LI-COR, Lincoln, NE, USA) in Tris buffer saline with 0.1% Tween 20 and staining with antibodies against GAA (ab137068, Abcam, Cambridge, MA, USA), or anti-GAPDH (ab9484, Abcam, Cambridge, MA, USA) and the appropriate secondary (926-32213 or 925-68070, LI-COR, Lincoln, NE, USA). Blots were imaged with a LI-COR Odyssey CLx.

Protein band intensity was quantified in LI-COR Image Studio software. The quantification of the mature 77 kDa GAA band for each sample was determined by first normalizing to the lane's TPS signal, then normalizing to GAA levels in the serum (loading control and liver expression control, respectively). Values were then compared to the positive control group anti-mouse TFRC scfv:hGAA in Wt mice, and negative control group anti-mTFRC scfv:hGAA in Tfrc^{hum/hum} mice (FIGS. 2A-2C, Table 4-1).

TABLE 4-1
Quantification of mature hGAA protein in brain homogenate
from mice treated HDD with anti-hTFRC scfv:hGAA plasmids

| | | Mature hGAA protein in brain |
|---|---|---|
| Treatment group | Genotype | (normalized to positive control) |
| anti-mTFRCscfv:hGAA | Wt | 1.00 ± 0.43* |
| (positive control) | | |
| anti-mTFRCscfv:hGAA | Tfrc^{hum/hum} | 0.02 ± 0.03 |
| (negative control) | | |
| 69261scfv:hGAA | Tfrc^{hum/hum} | 0.67 ± 0.50 |
| 69307scfv:hGAA | Tfrc^{hum/hum} | 1.08 ± 0.19 |
| 69323scfv:hGAA | Tfrc^{hum/hum} | 0.91 ± 0.46 |
| 69329scfv:hGAA | Tfrc^{hum/hum} | 0.65 ± 0.13 |
| 69340scfv:hGAA | Tfrc^{hum/hum} | 0.55 ± 0.58 |
| 69348scfv:hGAA | Tfrc^{hum/hum} | 0.50 ± 0.05 |
| 12795scfv:hGAA | Tfrc^{hum/hum} | 0.27 ± 0.20 |
| 12798scfv:hGAA | Tfrc^{hum/hum} | 0.72 ± 0.42 |
| 12799scfv:hGAA | Tfrc^{hum/hum} | 1.05 ± 0.51* |
| 12801scfv:hGAA | Tfrc^{hum/hum} | 0.49 ± 0.18 |
| 12802scfv:hGAA | Tfrc^{hum/hum} | 0.29 ± 0.27 |
| 12839scfv:hGAA | Tfrc^{hum/hum} | 1.29 ± 0.27** |
| 12841scfv:hGAA | Tfrc^{hum/hum} | 1.72 ± 0.06*** |
| 12843scfv:hGAA | Tfrc^{hum/hum} | 1.79 ± 0.85*** |
| 12845scfv:hGAA | Tfrc^{hum/hum} | 3.08 ± 0.92*** |
| 12847scfv:hGAA | Tfrc^{hum/hum} | 1.24 ± 0.30 |
| 12848scfv:hGAA | Tfrc^{hum/hum} | 0.59 ± 0.16 |
| 12850scfv:hGAA | Tfrc^{hum/hum} | 0.47 ± 0.05 |

Data quantified from western blot as arbitrary units (FIGS. 2A-2C). All values are mean ± SD, n = 3-6 per group.
One Way ANOVA vs. negative control anti-mTFRC scfv:hGAA in Tfrc^{hum/hum} mice;
*p < 0.05;
**p < 0.005;
***p < 0.0001

The control anti-mTRFC that was conjugated to GAA was 8D3 scFv. The 8D3 scFv has the heavy chain amino acid sequence:

(SEQ ID NO: 326)
EVQLVESGGGLVQPGNSLTLSCVASGFTFSNYGMHWIRQAPKKGLEWIA
MIYYDSSKMNYADTVKGRFTISRDNSKNTLYLEMNSLRSEDTAMYYCAV
PTSHYVVDVWGQGVSVTVSS,

and the light chain amino acid sequence:

(SEQ ID NO: 327)
DIQMTQSPASLSASLEEIVTITCQASQDIGNWLAWYQQKPGKSPQLLIY
GATSLADGVPSRESGSRSGTQFSLKISRVQVEDIGIYYCLQAYNTPWTF
GGGTKLELK.

Capillary Depletion of Brain Samples Following HDD of Anti-hTFRC Scfv:hGAA Plasmids

Anti-hTFRC scfv:hGAA molecules from Table 4-1 were tested in a secondary screen in Tfrc^{hum} mice to determine whether hGAA was present in the brain parenchyma, and not trapped in the BBB endothelial cells. Four molecules (12799, 12839, 12843, and 12847) identified in screen as being present in parenchyma based on mature hGAA in the parenchyma fraction on Western blot, as well as high affinity to cyno TFRC.

Animals were treated HDD as detailed above. Forty-eight hours post-injection, mice were perfused with 30 mL 0.9% saline immediately after sacrifice by CO₂ asphyxiation. A 2 mm coronal slice of cerebrum was taken between bregma and −2 mm bregma and placed in 700 uL physiological buffer (10 mM HEPES, 4 mM KCl, 2.8 mM CaCl₂), 1 mM MgSO₄, 1 mM NaH₂PO₄, 10 mM D-glucose in 0.9% saline pH 7.4) on ice. Brain slices were gently homogenized on ice with a glass dounce homogenizer. An equivalent volume of 26% dextran (MW 70,000 Da) in physiological buffer was added (final 13% dextran) and homogenized 10 more strokes. Parenchyma (supernatant) and endothelial (pellet) fractions were separated by centrifugation at 5,400 g for 15 min at 4° C. Anti-hGAA western blot was performed on fractions as detailed above (FIG. 3, Table 4-2). Blots were also probed with anti-CD31 endothelial marker (Abcam ab182982).

TABLE 4-2
Quantification of mature hGAA protein in brain parenchyma fractions and BBB
endothelial fractions of mice treated HDD with anti-hTFRC scfv:hGAA plasmids

| | | Mature hGAA protein | Mature hGAA protein | Affinity to |
|---|---|---|---|---|
| | | in brain parenchyma | in brain endothelium | mfTFRC |
| | | (normalized to | (normalized to | (% of |
| Treatment group | Genotype | positive control) | positive control) | hTFRC binding) |
| anti- | Wt | 1.00 | 5.82 | ND |
| mTFRCscfv:hGAA | | | | |
| (positive control) | | | | |
| anti- | Tfrc^{hum/hum} | 0.00 | 0.01 | ND |
| mTFRCscfv:hGAA | | | | |
| (negative control) | | | | |
| 69307scfv:hGAA | Tfrc^{hum/hum} | 1.24 | 10.73 | 0% |
| 69323scfv:hGAA | Tfrc^{hum/hum} | 0.62 | 4.18 | 7% |
| 12798scfv:hGAA | Tfrc^{hum/hum} | 0.91 | 8.37 | 34% |
| 12799scfv:hGAA | Tfrc^{hum/hum} | 0.44 | 3.99 | 126% |
| 12839scfv:hGAA | Tfrc^{hum/hum} | 0.55 | 0.84 | 78% |
| 12841scfv:hGAA | Tfrc^{hum/hum} | 0.78 | 4.23 | 8% |
| 12843scfv:hGAA | Tfrc^{hum/hum} | 1.13 | 12.99 | 75% |
| 12845scfv:hGAA | Tfrc^{hum/hum} | 2.04 | 13.06 | 25% |
| 12847scfv:hGAA | Tfrc^{hum/hum} | 0.60 | 4.96 | 102% |
| 12848scfv:hGAA | Tfrc^{hum/hum} | 0.17 | 1.24 | 29% |
| 12850scfv:hGAA | Tfrc^{hum/hum} | 0.22 | 2.25 | 13% |

hGAA protein quantified from western blot as arbitrary units (FIG. 3). n = 1 per group. Affinity to cynomolgus macaque TFRC Luminex data, calculated as percent of binding to hTFRC: (mfTFRC binding ÷ hTFRC binding) × 100

TABLE 4-3
Quantification of hGAA protein in quadricep of mice
treated HDD with anti-hTFRC scfv:hGAA plasmids

| | | hGAA protein in quadricep |
|---|---|---|
| Treatment group | Genotype | (normalized to positive control) |
| Saline (vehicle) | Tfrc^{hum/hum} | 0.38 ± 0.25 |
| anti-mTFRCscfv:hGAA | Wt | 1.07 ± 0.27 |
| (positive control) | | |
| anti-mTFRCscfv:hGAA | Tfrc^{hum/hum} | 0.56 ± 0.17 |
| (negative control) | | |
| 69307scfv:hGAA | Tfrc^{hum/hum} | 0.58 ± 0.18 |
| 69323scfv:hGAA | Tfrc^{hum/hum} | 1.10 ± 0.19 |
| 12798scfv:hGAA | Tfrc^{hum/hum} | 1.33 ± 0.56 |
| 12799scfv:hGAA | Tfrc^{hum/hum} | 0.67 ± 0.18 |
| 12839scfv:hGAA | Tfrc^{hum/hum} | 1.80 ± 0.18 |
| 12841scfv:hGAA | Tfrc^{hum/hum} | 1.15 ± 0.12 |
| 12843scfv:hGAA | Tfrc^{hum/hum} | 1.78 ± 0.43 |
| 12845scfv:hGAA | Tfrc^{hum/hum} | 1.70 ± 1.33 |
| 12847scfv:hGAA | Tfrc^{hum/hum} | 7.74 ± 9.42 |
| 12848scfv:hGAA | Tfrc^{hum/hum} | 0.82 ± 0.18 |
| 12850scfv:hGAA | Tfrc^{hum/hum} | 0.76 ± 0.34 |

Capillary Depletion of Mouse Brain Samples Following Liver-Depot AAV8 Anti-hTFRC Scfv:hGAA Treatment

To confirm our HDD screen findings in a more long-term treatment model, we treated Tfrc^{hum} mice with anti-hTFRC scfv:hGAA molecules delivered as episomal liver depot AAV8 anti-hTFRC scfv:GAA under the TTR promoter. We found that all 4 molecules (12799, 12843, 12847 and 12839) delivered mature hGAA to the brain parenchyma when delivered as AAV8.

### AAV Production and In Vivo Transduction

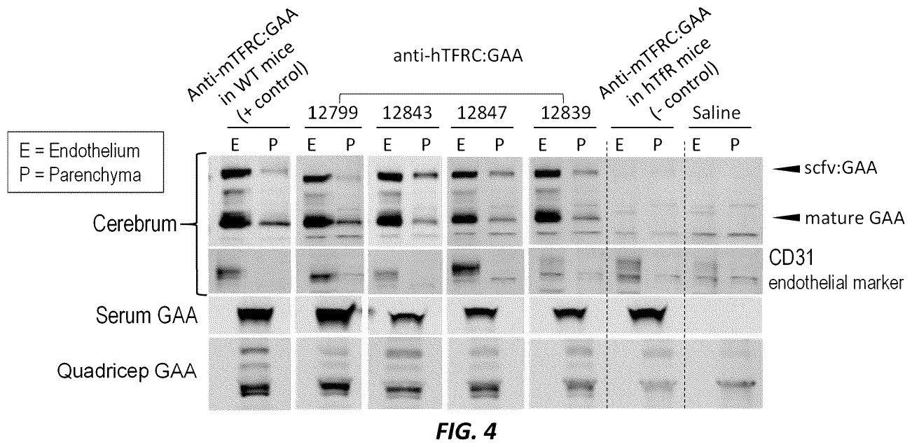

Recombinant AAV8 (AAV2/8) was produced in HEK293 cells. Cells were transfected with three plasmids encoding adenovirus helper genes, AAV8 rep and cap genes, and recombinant AAV genomes containing transgenes flanked by AAV2 inverted terminal repeats (ITRs). On day 5, cells and medium were collected, centrifuged, and processed for AAV purification. Cell pellets were lysed by freeze-thaw and cleared by centrifugation. Processed cell lysates and medium were overlaid onto iodixanol gradients columns and centrifuged in an ultracentrifuge. Virus fractions were removed from the interface between the 40% and 60% iodixanol solutions and exchanged into 1×PBS with desalting columns. AAV vg (vg=viral genomes) were quantified by ddPCR. AAVs were diluted in PBS+0.001% F-68 Pluronic immediately prior to injection. Tfrc^{hum} mice were dosed with 3e12vg/kg body weight in a volume of −100 uL. Mice were sacrificed 4 weeks post injection and capillary depletion and western blotting were performed as described above (FIG. 4, Table 4-4).

TABLE 4-4
Quantification of mature hGAA protein in brain parenchyma fractions and BBB endothelial
fractions of mice treated with liver-depot AAV8 anti-hTFRC scfv:hGAA

| | | Mature hGAA protein in brain | Mature hGAA protein in brain |
|---|---|---|---|
| | | parenchyma (normalized to | endothelium (normalized to |
| Treatment group | Genotype | positive control) | positive control) |
| anti-mTFRCscfv:hGAA | Wt | 1.00 | 1.00 |
| (positive control) | | | |
| anti-mTFRCscfv:hGAA | Tfrc^{hum/hum} | 0.02 | 0.01 |
| (negative control) | | | |
| 12799scfv:hGAA | Tfrc^{hum/hum} | 0.94 | 0.94 |
| 12839scfv:hGAA | Tfrc^{hum/hum} | 0.49 | 0.62 |
| 12843scfv:hGAA | Tfrc^{hum/hum} | 0.61 | 0.63 |
| 12847scfv:hGAA | Tfrc^{hum/hum} | 1.90 | 1.33 |

Data quantified from western blot as arbitrary units (FIG. 4). n = 1 per group

Rescue of Glycogen Storage Phenotype in Gaa^{-/-}/Tfrc^{hum} Mice with AAV8 Episomal Liver Depot Anti-hTFRC Scfv:GAA

Anti-hTFRC scfv:GAA molecules in Pompe disease model mice were tested to determine whether anti-hTFRCscfv:GAA rescued the glycogen storage phenotype. The molecules, 12839, 12843, 12847, normalized glycogen to Wt levels. (12799 not tested).

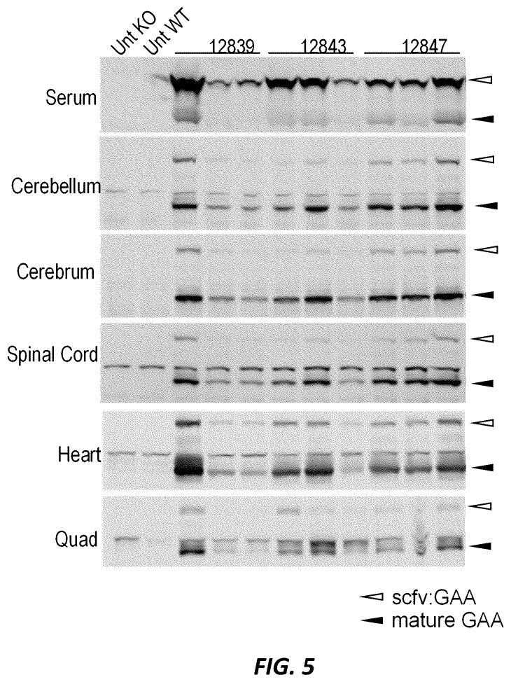

AAV production and in vivo transduction were performed as above. Gaa^{-/-}/Tfrc^{hum} mice were dosed with 2e12vg/kg AAV8. Tissues were harvested 4 weeks post-injection and flash-frozen as above. hGAA Western blot was performed as above (FIG. 5, Table 4-5).

### Glycogen Quantification

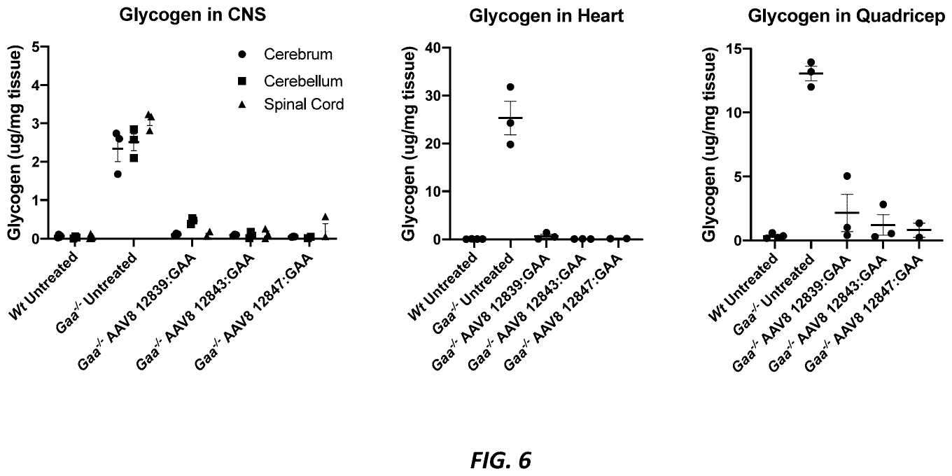

Tissues were dissected from mice immediately after sacrifice by CO₂ asphyxiation, snap frozen in liquid nitrogen, and stored at −80° C. Tissues were lysed on a benchtop homogenizer with stainless steel beads in distilled water for glycogen measurements or RIPA buffer for protein analyses. Glycogen analysis lysates were boiled and centrifuged to clear debris. Glycogen measurements were performed fluorometrically with a commercial kit according to manufacturer's instructions (K646, BioVision, Milpitas, CA, USA). See Table 4-6 and FIG. 6.

TABLE 4-5
Quantification of hGAA protein in tissues of Gaa^{-/-}/Tfrc^{hum} mice
treated with liver-depot AAV8 anti-hTFRC scfv:hGAA

| | | | | | | Spinal | | |
|---|---|---|---|---|---|---|---|---|
| Treatment group | n | Serum* | Liver* | Cerebrum** | Cerebellum** | Cord** | Heart** | Quadricep** |
| Gaa^{-/-} Untreated | 1 | 0.00 | 0.02 | 0.00 | 0.00 | 0.00 | 0.02 | 0.01 |
| Gaa^{-/-} | 3 | 2.42 ± | 1.63 ± | 0.14 ± 0.12 | 0.13 ± 0.12 | 0.19 ± | 0.53 ± | 0.14 ± 0.16 |
| 12839scfv:hGAA | | 2.41 | 0.96 | | | 0.19 | 0.52 | |
| Gaa^{-/-} | 3 | 2.07 ± | 2.23 ± | 0.17 ± 0.07 | 0.11 ± 0.05 | 0.17 ± | 0.49 ± | 0.18 ± 0.06 |
| 12843scfv:hGAA | | 1.35 | 0.08 | | | 0.09 | 0.31 | |
| Gaa^{-/-} | 3 | 1.56 ± | 1.40 ± | 0.25 ± 0.04 | 0.21 ± 0.09 | 0.42 ± | 0.58 ± | 0.19 ± 0.08 |
| 12847scfv:hGAA | | 0.71 | 0.13 | | | 0.19 | 0.17 | |

Data quantified from western blot as arbitrary units (FIG. 5). All values are mean ± SD, n = 1-3 per group.
*Total hGAA protein;
**Mature hGAA protein

TABLE 4-6
Quantification of glycogen in tissues of Gaa^{-/-}/Tfrc^{hum}
mice treated with liver-depot AAV8 anti-hTFRC scfv:hGAA

| Treatment group | Cerebrum | Cerebellum | Spinal Cord | Heart | Quadricep |
|---|---|---|---|---|---|
| Wt Untreated | 0.06 ± 0.04* | 0.01 ± 0.04* | 0.05 ± 0.05* | 0.08 ± 0.02* | 0.34 ± 0.19* |
| Gaa−/− Untreated | 2.34 ± 0.58 | 2.51 ± 0.38 | 3.08 ± 0.23 | 25.30 ± 6.06 | 13.05 ± 0.98 |
| Gaa−/− 12839scfv:hGAA | 0.11 ± 0.03* | 0.46 ± 0.08* | 0.08 ± 0.10* | 0.68 ± 0.68* | 2.15 ± 2.52* |
| Gaa−/− 12843scfv:hGAA | 0.09 ± 0.02* | 0.09 ± 0.08* | 0.13 ± 0.13* | 0.09 ± 0.01* | 1.22 ± 1.39* |
| Gaa−/− 12847scfv:hGAA | 0.05 ± 0.01* | 0.02 ± 0.03* | 0.20 ± 0.33* | 0.11 ± 0.11* | 0.80 ± 0.79* |

All values are glycogen ug/mg tissue, mean ± SD, n = 3-4 per group. One Way ANOVA
*p < 0.0001 vs. Gaa^{-/-} Untreated group

Rescue of Glycogen Storage in Brain and Muscle in Gaa^{-/-}/Tfrc^{hum} Mice with AAV8 Episomal Liver Depot Anti-hTFRC Scfv:GAA

Anti-hTFRCscfv:GAA molecules, 12799, 12843, and 12847, were tested in Pompe disease model mice to determine whether they rescued the glycogen storage phenotype. Histology was performed on brain and muscle sections to visualize glycogen in the tissues. All 3 molecules reduced glycogen staining in the brain and muscle.

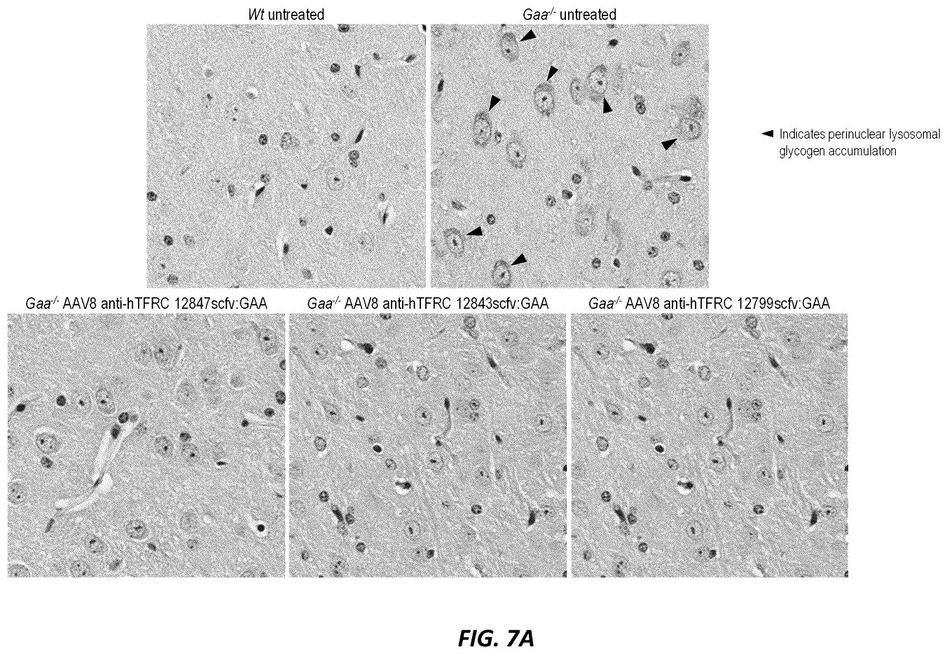

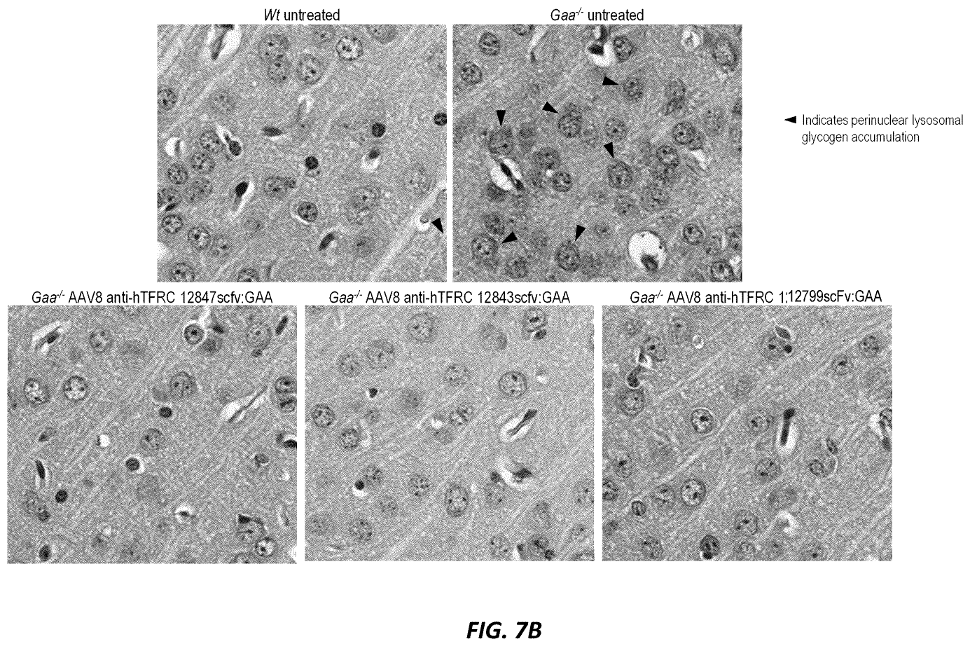

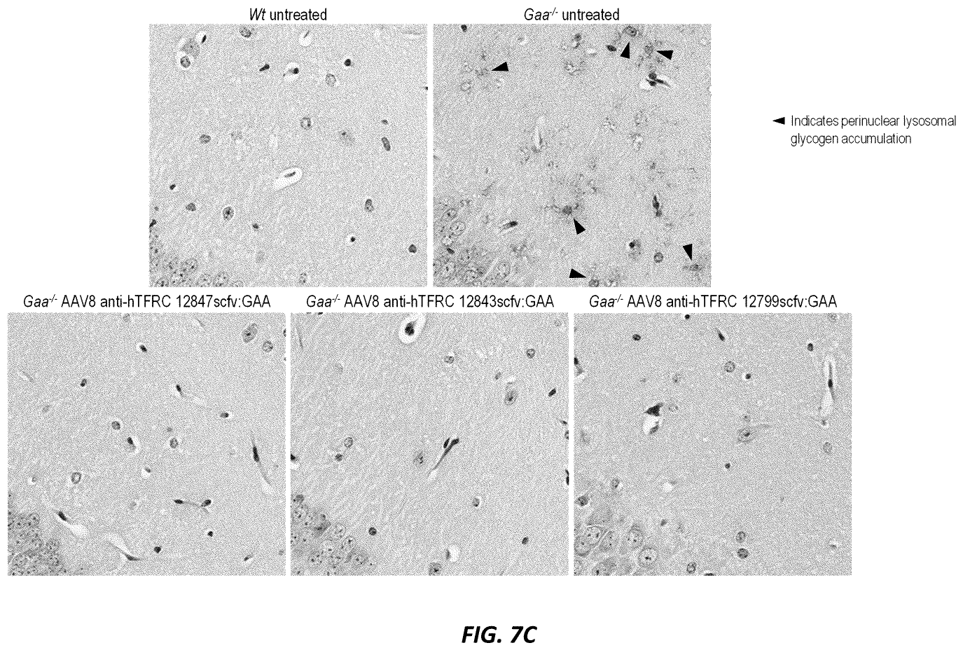

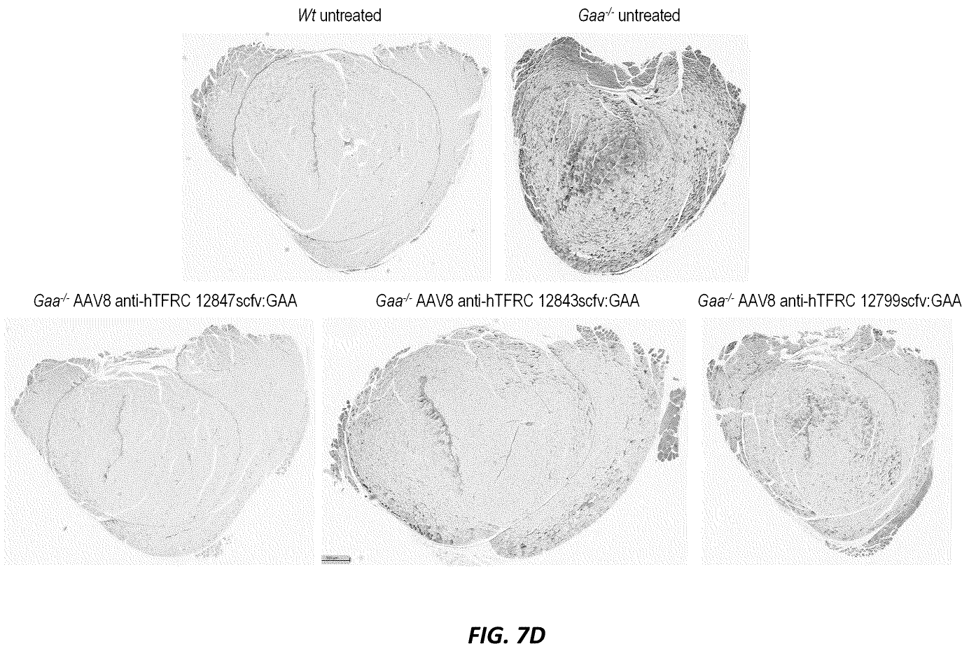

AAV production and in vivo transduction were performed as above. Three month old Gaa^{-/-}/Tfrc^{hum} mice were dosed with 4e11vg/kg AAV8. Four weeks post-injection, tissues were frozen for glycogen analysis as above (Table 4-7). For histology, animals were perfused with saline (0.9% NaCl), and tissues were drop-fixed overnight in 10% Normal Buffered Formalin. Tissues were washed 3× in PBS and stored in PBS/0.01% sodium azide until embedding. Tissues were embedded in paraffin and Sum sections were cut from brain (coronal, −2 mm bregma) and quadricep (fiber cross-section). Sections were stained with Periodic Acid-Schiff and Hematoxylin using standard protocols (FIGS. 7A-7D).

TABLE 4-7
Quantification of glycogen in tissues of Gaa^{-/-}/Tfrc^{hum}
mice treated with liver-depot AAV8 anti-hTFRC scfv:hGAA

| Treatment group | Cerebellum | Quadricep |
|---|---|---|
| Wt Untreated | 0.02 ± 0.03* | 0.55 ± 0.10* |
| Gaa−/− Untreated | 1.91 ± 0.26 | 12.19 ± 3.02 |
| Gaa−/− 12799scfv:hGAA | 0.10 ± 0.06* | 1.34 ± 0.9* |
| Gaa−/− 12843scfv:hGAA | 0.09 ± 0.06* | 1.09 ± 1.27* |
| Gaa−/− 12847scfv:hGAA | 0.07 ± 0.06* | 0.72 ± 0.64* |

All values are glycogen ug/mg tissue, mean ± SD, n = 5-8 per group. One Way ANOVA
*p < 0.0001 vs. Gaa^{-/-} Untreated group

### Example 5. Iron Assay

This Example evaluated the effect of anti-TfR antigen-binding proteins on iron homeostasis in mice.

### Validating TFRC Expression in Tfrc^{hum} Mice and Assessing Iron Homeostasis

To validate that Tfrc^{hum} mice expressed TFRC at physiological levels and had normal iron homeostasis, we compared Tfrc^{hum} mice to Wt mice and quantified expression of TFRC in tissues, serum markers, tissue iron content, and transferrin in tissues. Overall, TFRC expression and iron homeostasis was normal in the Tfrc^{hum} mice.

Six month old Wt mice (11 males, 4 females) and Tfrc^{hum} mice (10 males, 8 females) were analyzed in this experiment. Tissues were dissected from mice immediately after sacrifice by CO₂ asphyxiation, snap frozen in liquid nitrogen, and stored at −80° C.

Tfrc RNA Quantification by qPCR

Total RNA was isolated from tissues with Trizol following manufacturer protocol (ThermoFisher 15596026). Tfrc RNA was quantified by Taqman qPCR (ThermoFisher) following standard protocols using universal primers to exon 1 that amplify from both Wt and and Tfrc^{hum} mice (GCTGCATTGCGGACTGTAGA (SEQ ID NO: 503)/TCCATCATTCTCAGCTGCTACAA (SEQ ID NO: 504)). ΔΔCT values were calculated relative to the Wt male group. Data in Table 5-1.

### Serum Assays

Blood was collected from mice by cardiac puncture immediately following CO₂ asphyxiation and serum was separated using serum separator tubes (BD Biosciences, 365967). Serum iron and Total Iron Binding Content (TIBC) were quantified using standard protocols. Serum hepcidin was quantified by ELISA kit (Intrinsic Life Sciences SKU HMC-001). Data in Table 5-2.

### Tissue Iron Content

Wet tissue was weighed to achieve uniformity and then dried for 72 hours in an open tube at 56° C. Tissue was then placed in digestion buffer (10% Tricloroacetic acid and 37% HCL) and heated at 65° C. for 48 hours. To assay iron content, the supernatant was placed in a 96 well plate and incubated in a color development solution (Thioglycolic acid, bathophenanthroline acid and sodium acetate). Absorbance was read on a Spectramax i3 by Molecular Devices and Graph Pad Prism was used to interpolate the sample absorbance values read against a standard curve to calculate iron content in the whole piece of tissue. Iron content was then calculated based on dry weight. Data in Table 5-3.

### Transferrin ELISA

All tissues were homogenized using a Fastprep-24 5G from MP Biomedicals. Prior to homogenization, tissues were placed in RIPA buffer with phosphatase and HALT protease inhibitors (ThermoFisher), homogenized with their organ specific protocol and then centrifuged to pellet debris. The supernatant was collected and assayed for total protein using a Pierce BCA Protein Assay Kit. Absorbance was measured on a Spectramax i3 by Molecular Devices. Once total protein was measured, all samples were diluted to match the least concentrated sample so loading would be uniform for the ELISA. Kits obtained from Abcam were used to measure the presence of total transferrin in tissue homogenate (Abcam ab157724). Plates were run in accordance with the supplied protocol using the provided reagents and absorbance was read on a Spectramax i3 by Molecular Devices. Graph Pad Prism was used to interpolate the sample absorbance values read against a standard curve. Data in Table 5-4.

TABLE 5-1
Tfrc RNA quantification in untreated Wt and Tfrc^{hum} mice

| Genotype | Sex | Liver Tfrc | Quadricep Tfrc | Brain Tfrc |
|---|---|---|---|---|
| Wt | M | 1.02 ± 0.21 | 1.10 ± 0.53 | 1.02 ± 0.21 |
| Wt | F | 1.11 ± 0.64 | 0.60 ± 0.17 | 1.03 ± 0.13 |
| Tfrc^{hum} | M | 1.14 ± 0.28 | 1.02 ± 0.39 | 1.86 ± 0.35* |
| Tfrc^{hum} | F | 0.75 ± 0.22 | 0.43 ± 0.93 | 1.88 ± 0.25* |

All values are ΔΔCT vs. Wt males group, mean ± SD, n = 4-11 per group. One Way ANOVA
*p < 0.001 vs. Wt sex-matched group

TABLE 5-2
Serum iron markers in untread Wt and Tfrc^{hum} mice

| | | Serum Iron | Serum TIBC | Serum Hepcidin |
|---|---|---|---|---|
| Genotype | Sex | ug/dL | ug/dL | ng/mL |
| Wt | M | 146.73 ± 20.30 | 360.18 ± 27.02 | 416.73 ± 133.04 |
| Wt | F | 125.50 ± 9.04 | 342.25 ± 22.25 | 436.35 ± 143.28 |
| Tfrc^{hum} | M | 173.00 ± 12.77* | 351.20 ± 21.94 | 415.86 ± 101.27 |
| Tfrc^{hum} | F | 156.75 ± 14.18* | 353.50 ± 17.03 | 523.30 ± 175.70 |

All values are mean ± SD, n = 4-11 per group. All values are within normal physiological range. One Way ANOVA
*p < 0.05 vs. Wt sex-matched group

TABLE 5-3
Tissue iron quantification in untread Wt and Tfrc^{hum} mice

| Genotype | Sex | Liver | Spleen |
|---|---|---|---|
| Wt | M | 307.03 ± 32.74 | 1666.38 ± 239.18 |
| Wt | F | 507.45 ± 110.45 | 1833.12 ± 173.36 |
| Tfrc^{hum} | M | 300.00 ± 33.77 | 1818.44 ± 276.86 |
| Tfrc^{hum} | F | 638.46 ± 139.03 | 1695.96 ± 140.02 |

All values are ug/g dry tissue, mean ± SD, n = 4-11 per group. Values are non-significant (One Way ANOVA vs. Wt sex-matched group).

TABLE 5-4
Transferrin protein in untreated Wt and Tfrc^{hum} mice (ELISA)

| Genotype | Sex | Serum | Liver | Cerebrum |
|---|---|---|---|---|
| Wt | M | 1191.28 ± 137.03 | 32.61 ± 9.87 | 7.35 ± 1.30 |
| Wt | F | 1270.81 ± 138.42 | 27.01 ± 13.22 | 6.33 ± 0.93 |
| Tfrc^{hum} | M | 1251.40 ± 113.59 | 32.97 ± 7.26 | 7.92 ± 1.63 |
| Tfrc^{hum} | F | 1425.89 ± 290.77 | 40.17 ± 8.22 | 8.26 ± 2.08 |

All values are ug/mL homogenate normalized to protein content; mean ± SD, n = 4-11 per group. Values are non-significant (One Way ANOVA vs. Wt sex-matched group).

Rescue of Glycogen Storage in Brain and Muscle in Gaa^{-/-}/Tfrc^{hum} Mice with AAV8 Episomal Liver Depot Anti-hTFRC Scfv:GAA

We tested the anti-hTFRC scfv:GAA leads 12799, 12843, and 12847 in Pompe disease model mice to determine whether anti-hTFRC scfv:GAA rescued the glycogen storage phenotype (glycogen data in other data package). Here we also tested whether treatment with anti-TFRCscfv:GAA leads altered iron homeostasis (Tables 5-5, 5-6 and 5-7). We found that 4-week treatment did not affect iron homeostasis with any of the leads.

TABLE 5-5
Serum iron markers in Gaa^{-/-}/Tfrc^{hum} mice treated
with AAV8 episomal liver depot anti-hTFRC scfv:GAA

| | Serum iron | Serum TIBC | Serum Hepcidin |
|---|---|---|---|
| Treatment group | ug/dL | ug/dL | ng/mL |
| Wt Untreated | 203.83 ± 29.49 | 334.33 ± 17.83 | 265.89 ± 60.71 |
| Gaa^{-/-} Untreated | 196.50 ± 25.15 | 326.50 ± 34.39 | 329.19 ± 124.11 |
| Gaa^{-/-} | 188.50 ± 32.83 | 319.14 ± 28.20 | 341.25 ± 104.87 |
| 12799scfv:hGAA | | | |
| Gaa^{-/-} | 163.63 ± 28.27 | 275.88 ± 65.67 | 298.47 ± 104.60 |
| 12843scfv:hGAA | | | |
| Gaa^{-/-} | 159.29 ± 19.09 | 323.00 ± 24.82 | 387.47 ± 69.56 |
| 12847scfv:hGAA | | | |

All values are mean ± SD, n = 5-8 per group. All values are non-significant (One Way ANOVA)

TABLE 5-6
Tissue iron quantification in Gaa^{-/-}/Tfrc^{hum} mice treated
with AAV8 episomal liver depot anti-hTFRC scfv:GAA

| Treatment group | Liver | Heart | Spleen |
|---|---|---|---|
| Wt Untreated | 228.12 ± 37.65 | 349.78 ± 27.98 | 893.68 ± 216.93 |
| Gaa^{-/-} Untreated | 260.59 ± 49.54 | 355.82 ± 48.43 | 1258.57 ± 600.35 |
| Gaa^{-/-} | 285.07 ± 67.17 | 350.44 ± 51.70 | 1251.36 ± 628.45 |
| 12799scfv:hGAA | | | |
| Gaa^{-/-} | 279.64 ± 41.89 | 360.78 ± 37.34 | 906.81 ± 280.82 |
| 12843scfv:hGAA | | | |
| Gaa^{-/-} | 336.33 ± 85.74 | 391.67 ± 58.36 | 1773.74 ± 374.26 |
| 12847scfv:hGAA | | | |

All values are ug/g dry tissue, mean ± SD, n = 5-8 per group. All values are non-significant (One Way ANOVA).

TABLE 5-7
Transferrin protein in Gaa^{-/-}/Tfrc^{hum} mice treated with
AAV8 episomal liver depot anti-hTFRC scfv:GAA (ELISA)

| Treatment group | Liver | Spleen | Cerebrum |
|---|---|---|---|
| Wt Untreated | 19.82 ± 4.73 | 3.17 ± 1.46 | 10.69 ± 1.05 |
| Gaa^{-/-} Untreated | 14.71 ± 7.37 | 6.36 ± 2.59 | 12.54 ± 2.07 |
| Gaa^{-/-} 12799scfv:hGAA | 16.66 ± 6.99 | 5.87 ± 2.48 | 10.34 ± 1.49 |
| Gaa^{-/-} 12843scfv:hGAA | 14.16 ± 5.93 | 5.67 ± 1.95 | 11.19 ± 2.56 |
| Gaa^{-/-} 12847scfv:hGAA | 13.81 ± 3.04 | 5.70 ± 1.30 | 13.72 ± 1.87 |

All values are ug/mL homogenate normalized to protein content; mean ± SD, n = 5-8 per group. Values are non-significant (One Way ANOVA vs. Gaa^{-/-} Untreated group).

### Example 6. Insertion Anti-hTFRC:GAA Gene Therapy in Mice

### mAb Clone IDs

H1H12847B in scfv:GAA format (REGN16826)
12450NVH in scfv:GAA format (comparator, REGN5534)
   Insertion of Anti-hTFRC 12847Scfv:GAA in Gaa^{-/-}/Tfrc^{hum/hum} Mice

We tested our lead anti-hTFRC 12847scfv:GAA in Pompe disease model mice by albumin insertion to determine whether we could replicate the results we saw with episomal AAV8 liver depot expression. Albumin insertion of 12847scfv:GAA delivered mature hGAA protein to the brain and muscle, and rescued the glycogen storage phenotype in Gaa^{-/-}/Tfrc^{hum/hum} mice. These data were produced with the native 12847scfv:GAA sequence that is not optimized.

We compared 12847scfv:GAA to the muscle-targeted anti-hCD63scfv:GAA in Gaa^{-/-}/Cd63^{hum} mice. In this particular experiment, the expression of anti-hCD63scfv:GAA was lower than usual and does not deliver as much GAA protein to the muscle nor normalize glycogen as it usually does. This may make it appear that anti-hCD63scfv:GAA is less effective than 12847scfv:GAA in the muscle but in most experiments we found them to be comparable in the muscle.

### AAV Production

A promoterless AAV genome plasmid was created with the 12847scfv:GAA sequence and the mouse albumin exon 1 splice acceptor site at the 3′ end. Recombinant AAV8 (AAV2/8) was produced in HEK293 cells. Cells were transfected with three plasmids encoding adenovirus helper genes, AAV8 rep and cap genes, and recombinant AAV genomes containing transgenes flanked by AAV2 inverted terminal repeats (ITRs). On day 5, cells and medium were collected, centrifuged, and processed for AAV purification. Cell pellets were lysed by freeze-thaw and cleared by centrifugation. Processed cell lysates and medium were overlaid onto iodixanol gradients columns and centrifuged in an ultracentrifuge. Virus fractions were removed from the interface between the 40% and 60% iodixanol solutions and exchanged into 1×PBS with desalting columns. AAV vg were quantified by ddPCR.

In Vivo CRISPR/Cas9 Insertion into the Albumin Locus

Three month old Gaa^{-/-}/Tfrc^{hum/hum} mice were dosed via tail vein injection with 3e12vg/kg AAV8 12847scfv:GAA and 3 mg/kg LNP gRNA/Cas9 mRNA diluted in PBS+0.001% F-68 Pluronic. Mice were sacrificed 3 weeks post injection. Negative control mice received insertion AAV8 without LNP. Positive control mice were dosed with 4e11vg/kg episomal liver depot AAV8 12847scfv:GAA under the TTR promoter (phenotype rescue data previously shown). Tissues were dissected from mice immediately after sacrifice by CO₂ asphyxiation, snap frozen in liquid nitrogen, and stored at −80° C. Blood was collected from mice by cardiac puncture immediately following CO₂ asphyxiation and serum was separated using serum separator tubes (BD Biosciences, 365967).

TABLE 6-1
Treatment groups and controls

| Treatment group | Genotype | Function |
|---|---|---|
| Wt Untreated | Tfrc^{hum} | Normal untreated mouse control |
| Gaa^{-/-} untreated | Gaa^{-/-}/ | Untreated Pompe disease mouse |
| | Tfrc^{hum} | |
| Gaa^{-/-} insertion | Gaa^{-/-}/ | Negative control for insertion |
| AAV only | Tfrc^{hum} | (no Cas9/gRNA delivered) |
| Gaa^{-/-} episomal | Gaa^{-/-}/ | Positive control, previously |
| AAV8 TTR | Tfrc^{hum} | shown rescue of glycogen |
| 12847scfv:hGAA | | storage phenotype |
| Gaa^{-/-} insertion | Gaa^{-/-}/ | Experimental insertion group |
| 12847scfv:hGAA | Tfrc^{hum} | |
| Gaa^{-/-} untreated | Gaa^{-/-}/ | Untreated Pompe disease mouse |
| | Cd63^{hum} | (CD63 humanized) |
| Gaa^{-/-} insertion | Gaa^{-/-}/ | Negative control for BBB- |
| anti-CD63scfv:hGAA | Cd63^{hum} | crossing (muscle targeted) |

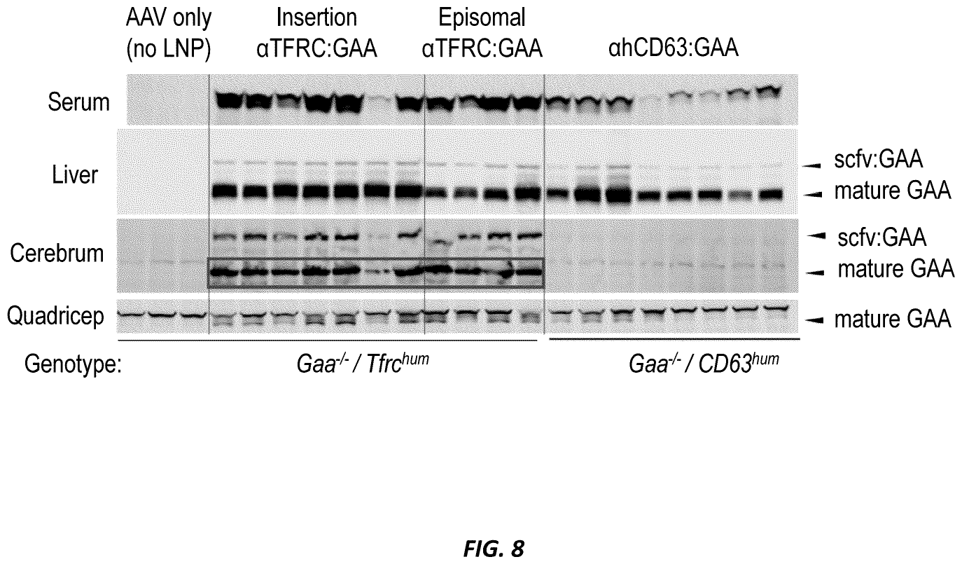

### Western Blot: (Table 6-2, FIG. 8)

Tissue lysates were prepared by lysis in RIPA buffer with protease inhibitors (1861282, Thermo Fisher, Waltham, MA, USA). Tissue lysates were homogenized with a bead homogenizer (FastPrep5, MP Biomedicals, Santa Ana, CA, USA). Cells or tissue lysates were run on SDS-PAGE gels using the Novex system (LifeTech Thermo, XPO4200BOX, LC2675, LC3675, LC2676). Gels were transferred to low-fluorescence polyvinylidene fluoridev (PVDF) membrane (IPFL07810, LI-COR, Lincoln, NE, USA) and stained with Revert 700 Total Protein Stain (TPS; 926-11010 LI-COR, Lincoln, NE, USA), followed by blocking with Odyssey blocking buffer (927-500000, LI-COR, Lincoln, NE, USA) in Tris buffer saline with 0.1% Tween 20 and staining with antibodies against GAA (ab137068, Abcam, Cambridge, MA, USA), or anti-GAPDH (ab9484, Abcam, Cambridge, MA, USA) and the appropriate secondary (926-32213 or 925-68070, LI-COR, Lincoln, NE, USA). Blots were imaged with a LI-COR Odyssey CLx.

Protein band intensity was quantified in LI-COR Image Studio software. The quantification of the mature 77 kDa GAA band for each sample was determined by normalizing to the lane's TPS signal (loading control).

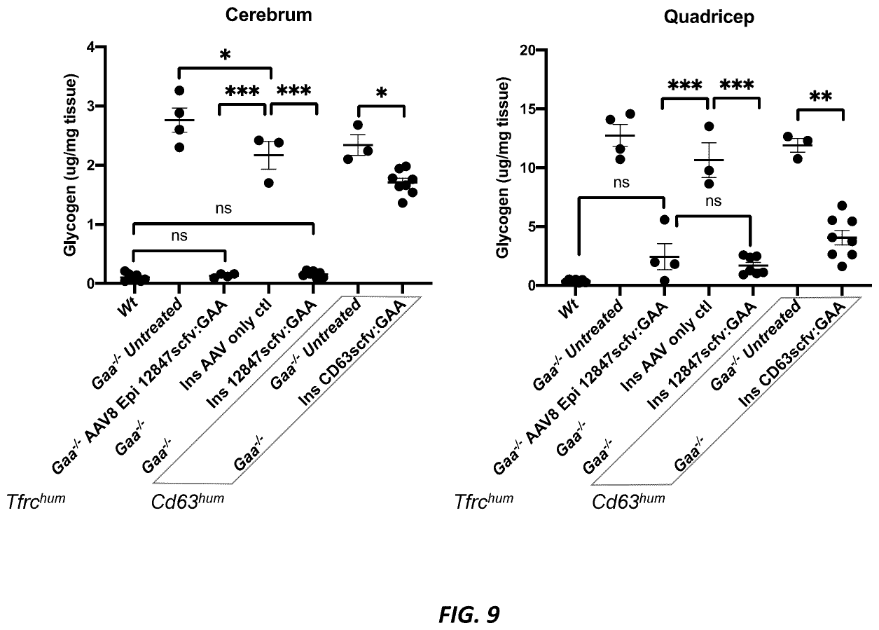

### Glycogen Quantification: (Table 6-3, FIG. 9)

Tissues were dissected from mice immediately after sacrifice by CO₂ asphyxiation, snap frozen in liquid nitrogen, and stored at −80° C. Tissues were lysed on a benchtop homogenizer with stainless steel beads in distilled water for glycogen measurements or RIPA buffer for protein analyses. Glycogen analysis lysates were boiled and centrifuged to clear debris. Glycogen measurements were performed fluorometrically with a commercial kit according to manufacturer's instructions (K646, BioVision, Milpitas, CA, USA).

TABLE 6-2
Quantification of hGAA protein in tissues of Gaa^{-/-}/Tfrc^{hum/hum}
mice treated with insertion anti-hTFRC 12847scfv:hGAA

| | Liver | Serum | Cerebrum | Quadricep |
|---|---|---|---|---|
| Treatment group | total hGAA | total hGAA | mature hGAA | mature hGAA |
| Gaa^{-/-} insertion AAV only negative control | 0.02 ± 0.003 | 0.03 ± 0.02 | 0.002 ± 0.001 | 0.006 ± 0.002 |
| Gaa^{-/-} episomal AAV8 TTR 12847scfv:hGAA | 2.35 ± 0.72 | 3.65 ± 2.09 | 0.49 ± 0.20^{§§} | 0.148 ± 0.043^{§§} |
| Gaa^{-/-} insertion 12847scfv:hGAA | 4.31 ± 0.87* | 3.47 ± 2.37 | 0.57 ± 0.26^{§§} | 0.141 ± 0.062^{§§} |
| Gaa^{-/-} insertion anti-CD63scfv:hGAA | 2.67 ± 1.04* | 0.93 ± 0.55* | 0.01 ± 0.003 | 0.060 ± 0.037 |

All values are arbitrary units, mean ± SD, n = 3-8 per group. One Way ANOVA
*p < 0.05 vs. Gaa^{-/-} episomal AAV8 TTR 12847scfv:GAA group;
^{§§}p < 0.001 vs. AAV only negative control group.

TABLE 6-3
Quantification of glycogen in tissues of Gaa^{-/-}/Tfrc^{hum/hum}
mice treated with insertion anti-hTFRC 12847scfv:hGAA

| Treatment group | Cerebrum | Quadricep |
|---|---|---|
| Wt untreated | 0.10 ± 0.07 | 0.37 ± 0.13 |
| Gaa^{-/-}/Tfrc^{hum} untreated | 2.76 ± 0.41 | 12.75 ± 1.88 |
| (Tfrc^{hum}) | | |
| Gaa^{-/-}/Tfrc^{hum} insertion | 2.17 ± 0.40* | 10.64 ± 2.56 |
| AAV only | | |
| Gaa^{-/-}/Tfrc^{hum} episomal | 0.13 ± 0.03***^{§} | 2.44 ± 2.21***^{§} |
| AAV8 TTR | | |
| 12847scfv:hGAA | | |
| Gaa^{-/-}/Tfrc^{hum} insertion | 0.16 ± 0.05***^{§} | 1.67 ± 0.76***^{§} |
| 12847scfv:hGAA | | |
| Gaa^{-/-}/Cd63^{hum} untreated | 2.34 ± 0.30 | 11.91 ± 1.01 |
| Gaa^{-/-}/Cd63^{hum} insertion | 1.71 ± 0.20* | 4.06 ± 0.13** |
| anti-CD63scfv:hGAA | | |

All values are glycogen ug/mg tissue, mean ± SD, n = 3-8 per group. One Way ANOVA
*p < 0.01 vs. Gaa^{-/-}/Cd63^{hum} untreated group;
**p < 0.001 vs. Gaa^{-/-}/Cd63^{hum} untreated group;
***p < 0.0001 vs. Gaa^{-/-}/Tfrc^{hum/hum} untreated group;
^{§}non-significant vs. Wt untreated group.

### Example 7. Anti-hTFRC:GAA Gene Insertion in Cynomolgus Monkeys

### mAb Clone IDs

H1H12847B in scfv:GAA format (REGN16826)
12450NVH in scfv:GAA format (comparator, REGN5534)

### Insertion of Anti-hTFRC 12847Scfv:GAA in Cynomolgus Monkeys

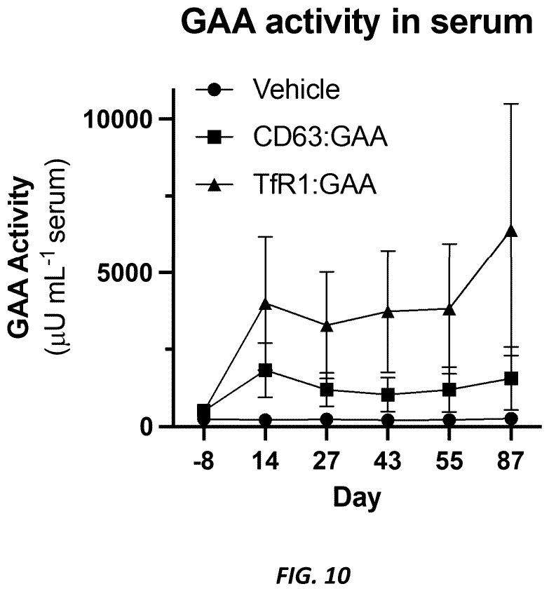

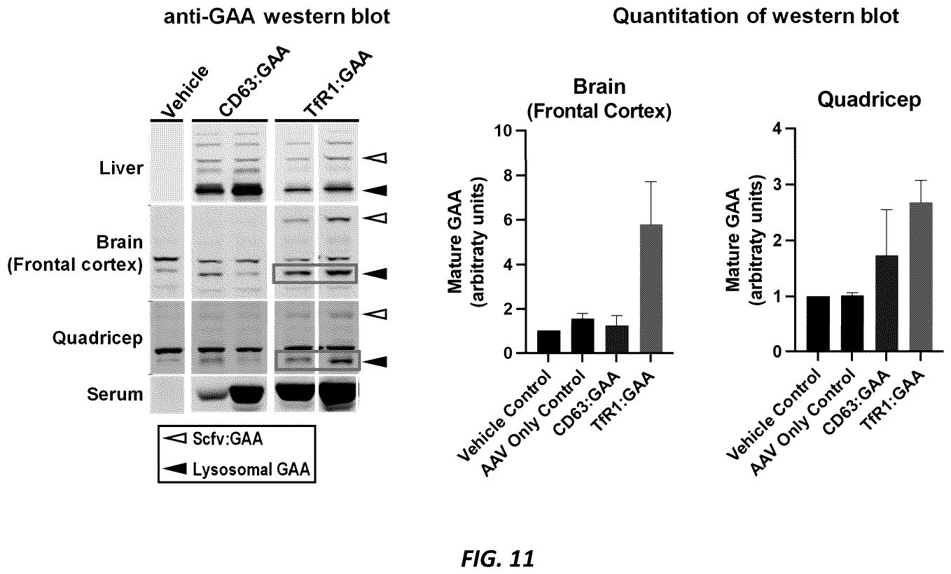

We tested our lead anti-hTFRC 12847scfv:GAA in cynomolgus monkeys by albumin insertion to determine whether we could replicate the results we saw in mice. We compared 12847scfv:GAA to the muscle-targeted anti-hCD63scfv:GAA in cynomolgus monkeys. As shown in FIGS. 10 and 11, serum GAA activity corresponded to serum GAA protein levels. As shown in FIG. 11, albumin insertion of 12847scfv:GAA delivered mature hGAA protein to the brain (frontal cortex) and muscle (quadricep).

Albumin insertion of anti-hCD63scfv:GAA or 12847scfv:GAA resulted in similar serum GAA levels with two different gRNAs, regardless of what gRNA was used (data not shown). Insertion did not negatively affect serum iron panel or creatinine (data not shown).

### AAV Production

A promoterless AAV genome plasmid was created with the 12847scfv:GAA sequence and the mouse albumin exon 1 splice acceptor site at the 3′ end. Recombinant AAV8 (AAV2/8) was produced in HEK293 cells. Cells were transfected with three plasmids encoding adenovirus helper genes, AAV8 rep and cap genes, and recombinant AAV genomes containing transgenes flanked by AAV2 inverted terminal repeats (ITRs). On day 5, cells and medium were collected, centrifuged, and processed for AAV purification. Cell pellets were lysed by freeze-thaw and cleared by centrifugation. Processed cell lysates and medium were overlaid onto iodixanol gradients columns and centrifuged in an ultracentrifuge. Virus fractions were removed from the interface between the 40% and 60% iodixanol solutions and exchanged into 1×PBS with desalting columns. AAV vg were quantified by ddPCR.

In Vivo CRISPR/Cas9 Insertion into the Albumin Locus

Cynomolgus monkeys age 2-3 years were dosed intravenously with 1.5e13vg/kg AAV8 12847scfv:GAA (or anti-CD63scfv:GAA) and 3 mg/kg LNP gRNA/Cas9 mRNA. Negative control monkeys received insertion AAV8 without LNP or vehicle control only. Serum and flash-frozen tissues were collected 90 days post-injection.

### GAA Activity in Serum: (FIG. 10)

Serum was collected prior to dosing and at indicated timepoints post-injection. GAA activity in the serum was quantified using Lysosomal alpha-Glucosidase Activity Assay Kit (Abcam ab252887). Serum GAA activity in CD63scfv:GAA and 12847scfv:GAA treated animals was above the vehicle controls and activity was similar between the treatment groups. Serum GAA activity corresponded with liver GAA protein expression and serum GAA protein levels (FIG. 11).

### Western Blot: (FIG. 11)

Tissue lysates were prepared by lysis in RIPA buffer with protease inhibitors (1861282, Thermo Fisher, Waltham, MA, USA). Tissue lysates were homogenized with a bead homogenizer (FastPrep5, MP Biomedicals, Santa Ana, CA, USA). Cells or tissue lysates were run on SDS-PAGE gels using the Novex system (LifeTech Thermo, XPO4200BOX, LC2675, LC3675, LC2676). Gels were transferred to low-fluorescence polyvinylidene fluoridev (PVDF) membrane (IPFL07810, LI-COR, Lincoln, NE, USA) and stained with Revert 700 Total Protein Stain (TPS; 926-11010 LI-COR, Lincoln, NE, USA), followed by blocking with Odyssey blocking buffer (927-500000, LI-COR, Lincoln, NE, USA) in Tris buffer saline with 0.1% Tween 20 and staining with antibodies against GAA (ab137068, Abcam, Cambridge, MA, USA), or anti-GAPDH (ab9484, Abcam, Cambridge, MA, USA) and the appropriate secondary (926-32213 or 925-68070, LI-COR, Lincoln, NE, USA). Blots were imaged with a LI-COR Odyssey CLx.

Protein band intensity was quantified in LI-COR Image Studio software. The quantification of the mature 77 kDa GAA band for each sample was determined by normalizing to the lane's TPS signal (loading control).

### Example 8. Epitope Mapping for Transferrin (TfR) Antibodies

Hydrogen-Deuterium Exchange Mass Spectrometry (HDX-MS) was performed to delineate regions in mouse and human Transferrin (m/hTfR) involved in binding of anti-Transferrin Receptor (TfR) antibodies. The anti-TfR monoclonal antibodies tested are described in Table 8-1. The reagents used and corresponding lot numbers are set forth in Table 8-2.

TABLE 8-1
Monoclonal Antibody Clones Tested

| REGN# | AbPID | Lot # |
|---|---|---|
| REGN17507 | H1H12798B | L1 |
| REGN17508 | H1H12799B | L1 |
| REGN17509 | H1H12835B | L1 |
| REGN17510 | H1H12839B | L1 |
| REGN17511 | H1H12841B | L1 |
| REGN17512 | H1H12843B | L1 |
| REGN17513 | H1H12845B | L1 |
| REGN17514 | H1H12847B | L1 |
| REGN17515 | H1H12848B | L1 |
| REGN17516 | H1H12850B | L1 |
| REGN17517 | H1H31874B | L1 |

TABLE 8-2
Reagents Used and Lot Numbers

| REGN# | Lot # | Description |
|---|---|---|
| REGN2120 | 03-121015 | hTfR(C89-F763).mmh |
| REGN2431 | L1 | hmm.hTfR(C89-F763) |

A general description of the HDX-MS method is set forth in, e.g., Ehring (1999) Analytical Biochemistry 267(2):252-259; and Engen and Smith (2001) Anal. Chem. 73:256A-265A. The experiment was performed on a customized HDX automation system (NovaBioAssays, MA) coupled to a Q Exactive HF mass spectrometer (Thermo Fisher Scientific, MA).

PBS-D₂O buffer was prepared by dissolving one PBS tablet in 100 mL 99.9% D₂O to form solution of 10 mM sodium phosphate, 137 mM NaCl, 3 mM KCl, pD 7.0 (equivalent to pH 7.4 at 25° C.). To initiate deuterium exchange, 10 μL of protein sample (hTfR alone, or hTfR in mixture with either of the monoclonal mAbs listed above, see, e.g., Table 8-1) was diluted with 90 μL PBS-D₂O buffer. After 5 minutes or 10 minutes, deuterium exchange was quenched by adding 100 μL quenching buffer (0.5 M TCEP, 4 M guanidine hydrochloride, pH 2.08) followed by 90 second incubation at 20° C. The quenched samples were digested by online pepsin/protease XIII column (NovaBioAssays, MA) at room temperature with 100 μL/min 0.1% formic acid in water. Peptic peptides were trapped by an ACQUITY UPLC Peptide BEH C18 VanGuard Pre-column (2.1×5 mm, Waters, MA) and further separated by an ACQUITY UPLC Peptide BEH C18 column (2.1×50 mm, Waters, MA) at −5° C., using 10-minute or 15-minute gradients with 0.1% formic acid in water and 0.1% formic acid in acetonitrile as mobile phases at 200 μL/min. Eluted peptides were analyzed by the mass spectrometer in LC-MS/MS or LC-MS mode.

A set of non-deuterated samples was prepared in PBS—H₂O buffer and analyzed with the method described above to identify peptide sequences and determine peptide masses without deuterium exchange. The LC-MS/MS data of non-deuterated samples were searched against a database containing sequences of hTfR, pepsin and protease XIII using the Byonic search engine (Protein Metrics, CA) with parameters for non-specific enzymatic digestion. The identified peptide list was then imported into the HDExaminer software (Sierra Analytics, CA) together with LC-MS data from all deuterated samples to calculate the deuterium uptake percentage (D %) of individual peptides from hTfR. Differences in deuterium uptake were calculated as ΔD %=D % of hTfR-mAb−D % of hTfR. Differences were considered significant if ΔD %<−5% (equivalent to |ΔD|>5% and ΔD %<0, averaged from 2 replicates). Mass spectra of peptides showing significant differences were examined manually to ensure that correct isotopic patterns were used for D % calculations by the software.

Two TfR protein constructs were used in HDX-MS experiments by reason of reagent availability and antibody specificity: hTfR(C89-F763).mmh, and hmm.hTfR(C89-F763). HDX data were obtained on 88%-95% of amino acids in hTfR with mmh tag. The numerical range provided before each amino acid sequence in the list below indicates the amino acid (aa) residue positions in hTfR which are protected by the indicated antibody. These amino acid residue positions are indicative of antibody binding sites on hTfR and does not provide residue-level contacts between them. Due to the nature of HDX-MS technique, the regions obtained by HDX-MS may be larger or smaller than actual contacts determined by high-resolution structural studies such as X-ray crystallography and cryogenic electron microscopy methods.

REGN17507 (H1H12798B) protects the following regions in hTfR:

(SEQ ID NO: 505)
146-167 LLNENSYVPREAGSQKDENLAL;
(SEQ ID NO: 506)
281-295 IYMDQTKEPIVNAEL;
and
(SEQ ID NO: 507)
572-576 TYKEL

REGN17508 (H1H12799B) protects the following regions in hTfR:

128-146
(SEQ ID NO: 508)
KRKLSEKLDSTDFTGTIKL;
503-522
(SEQ ID NO: 509)
YTLIEKTMQNVKHPVTGQFL;
and
576-592
(SEQ ID NO: 510)
LIERIPELNKVARAAAE.

REGN17509 (H1H12835B) protects the following region in hTfR:

147-165
(SEQ ID NO: 511)
LNENSYVPREAGSQKDENL.

REGN17510 (H1H12839B) protects the following region in hTfR:

238-246
(SEQ ID NO: 512)
GTKKDFEDL.

REGN17511 (H1H12841B) protects the following region in hTfR:

199-224
(SEQ ID NO: 513)
SVIIVDKNGRLVYLVENPGGYVAYSK.

REGN17512 (H1H12843B) protects the following region in hTfR:

146-164
(SEQ ID NO: 514)
LLNENSYVPREAGSQKDEN;
284-295
(SEQ ID NO: 515)
DQTKFPIVNAEL;
and
572-585
(SEQ ID NO: 516)
TYKELIERIPELNK.

REGN17513 (H1H12845B) protects the following region in hTfR:

199-222
(SEQ ID NO: 517)
SVIIVDKNGRLVYLVENPGGYVAY.

REGN17514 (H1H12847B) protects the following region in hTfR:

146-164
(SEQ ID NO: 514)
LLNENSYVPREAGSQKDEN;
and
572-585
(SEQ ID NO: 516)
TYKELIERIPELNK.

REGN17515 (H1H12848B) protects the following region in hTfR:

281-295
(SEQ ID NO: 506)
IYMDQTKFPIVNAEL;
and
346-365
(SEQ ID NO: 518)
FGNMEGDCPSDWKTDSTCRM.

REGN17516 (H1H12850B) protects the following region in hTfR:

146-167
(SEQ ID NO: 505)
LLNENSYVPREAGSQKDENLAL;
212-232
(SEQ ID NO: 520)
LVENPGGYVAYSKAATVTGKL;
281-297
(SEQ ID NO: 521)
IYMDQTKFPIVNAELSF;
337-345
(SEQ ID NO: 522)
ISRAAAEKL;
366-383
(SEQ ID NO: 523)
VTSESKNVKLTVSNVLKE;
and
557-572
(SEQ ID NO: 524)
FCEDTDYPYLGTTMDT

REGN17517 (H1H1874B) protects the following region in hTfR:

243-246
(SEQ ID NO: 519)
FEDL.

The minimal amino acid sequence in hTfR which is protected by the above-listed anti-TfR antibodies (i.e., the minimal epitope sequence), numerical range indicating the amino acid (aa) residue positions in hTfR which are protected each antibody, as well as the conformational or linear nature of each minimal epitope are described in Table 8-3. Each of the minimal epitopes is bound by its corresponding antibody at one or more amino acid residues within the minimal epitope sequence.

TABLE 8-3
Minimal epitope sequences in hTfR protected by anti-TfR antibodies.

| Antibody | Epitope | | Amino acid | | SEQ ID |
|---|---|---|---|---|---|
| ID | No. | Class | residue positions | Amino acid sequence | NO |
| REGN17507 | 1 | conformational | 146-149 | LLNE | 525 |
| (H1H12798B) | | | | | |
| REGN17507 | 2 | conformational | 572-576 | TYKEL | 507 |
| (H1H12798B) | | | | | |
| REGN17508 | 1 | conformational | 136-143 | DSTDFTGT | 526 |
| (H1H12799B) | | | | | |
| REGN17508 | 2 | conformational | 513-521 | VKHPVTGQF | 527 |
| (H1H12799B) | | | | | |
| REGN17508 | 3 | conformational | 577-583 | IERIPEL | 528 |
| (H1H12799B) | | | | | |
| REGN17509 | 1 | linear | 147-164 | LNENSYVPREAGSQKDEN | 529 |
| (H1H12835B) | | | | | |
| REGN17510 | 1 | linear | 243-246 | FEDL | 519 |
| (H1H12839B) | | | | | |
| REGN17511 | 1 | linear | 202-209 | IVDKNGRL | 530 |
| (H1H12841B) | | | | | |
| REGN17512 | 1 | conformational | 146-149 | LLNE | 525 |
| (H1H12843B) | | | | | |
| REGN17512 | 2 | conformational | 572-576 | TYKEL | 507 |
| (H1H12843B) | | | | | |
| REGN17513 | 1 | linear | 202-211 | IVDKNGRLVY | 531 |
| (H1H12845B) | | | | | |
| REGN17514 | 1 | conformational | 146-149 | LLNE | 525 |
| (H1H12847B) | | | | | |
| REGN17514 | 2 | conformational | 572-576 | TYKEL | 507 |
| (H1H12847B) | | | | | |
| REGN17515 | 1 | linear | 284-288 | DQTKF | 532 |
| (H1H12848B) | | | | | |
| REGN17516 | 1 | conformational | 212-218 | LVENPGGY | 533 |
| (H1H12850B) | | | | | |
| REGN17516 | 2 | conformational | 289-297 | PIVNAELSF | 534 |
| (H1H12850B) | | | | | |
| REGN17516 | 3 | conformational | 564-572 | PYLGTTMDT | 535 |
| (H1H12850B) | | | | | |
| REGN17517 | 1 | linear | 243-246 | FEDL | 519 |
| (H1H31874B) | | | | | |

The extracellular unit of hTfR is structurally categorized into three domains, the helical, protease-like and apical domains (PDB 1 SUV).

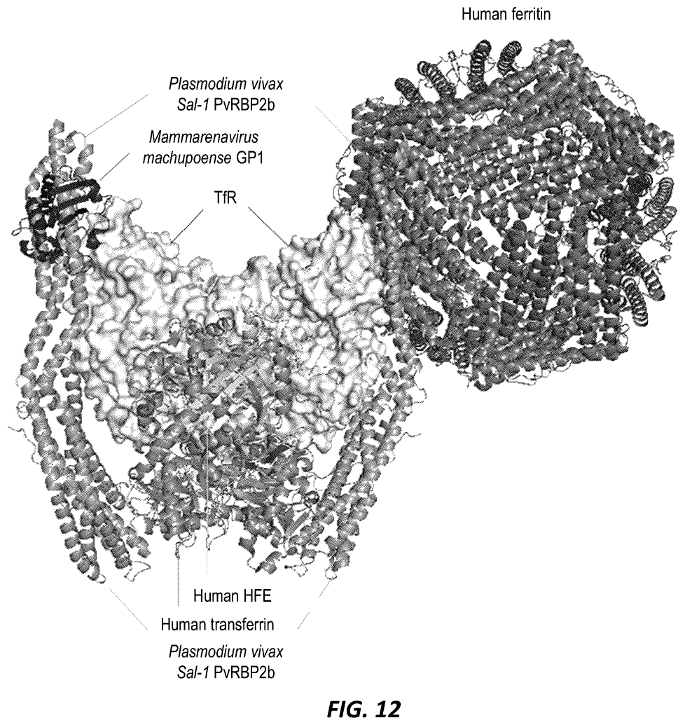

Structural studies of TfR in complex with a variety of molecules that have identified TfR binding sites, including Mammarenavirus machupoense GP1 protein (PDB 3KAS), canine parvovirus (PDB 2NSU), human ferritin (PDB 6GSR), Plasmodium vivax Sal-1 PvRBP2b (PDB 61D04), human HFE protein (PDB 1DE4), human transferrin (PDB 1 SUV), etc. FIG. 12 shows the interactions of the above-listed molecules superimposed on a single TfR molecule.

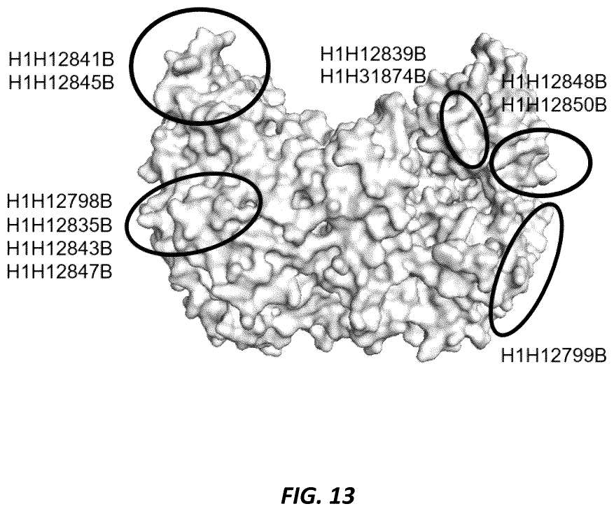

HDX protections for the antibodies tested in HDX-MS experiments can be assigned to 5 regions in TfR (PDB 1SUV) as depicted in FIG. 13.

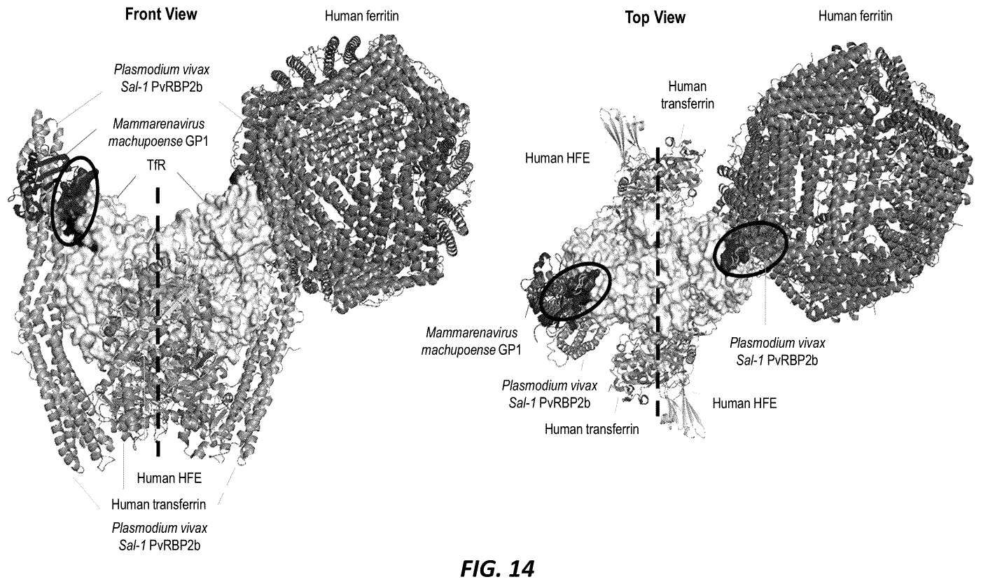

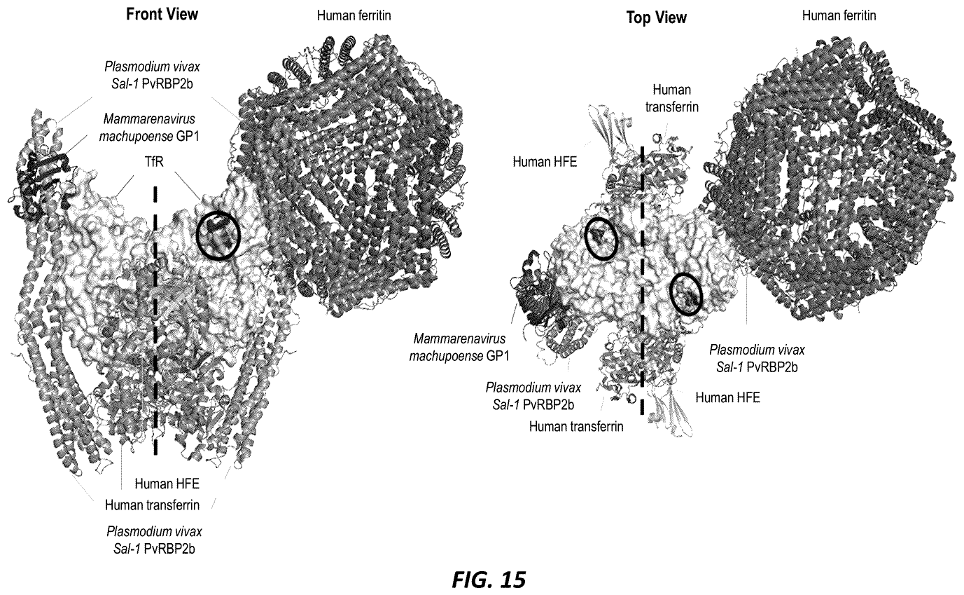

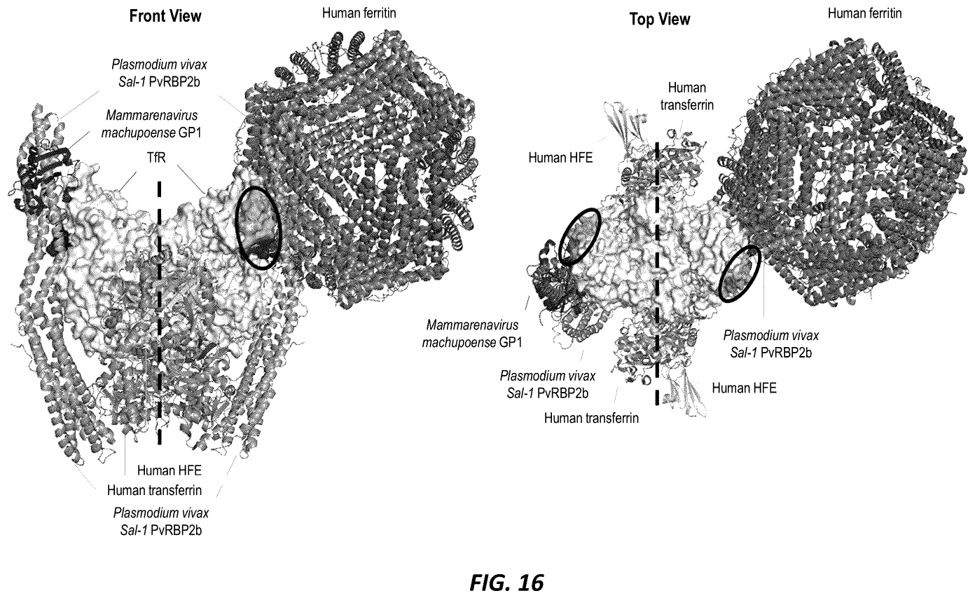

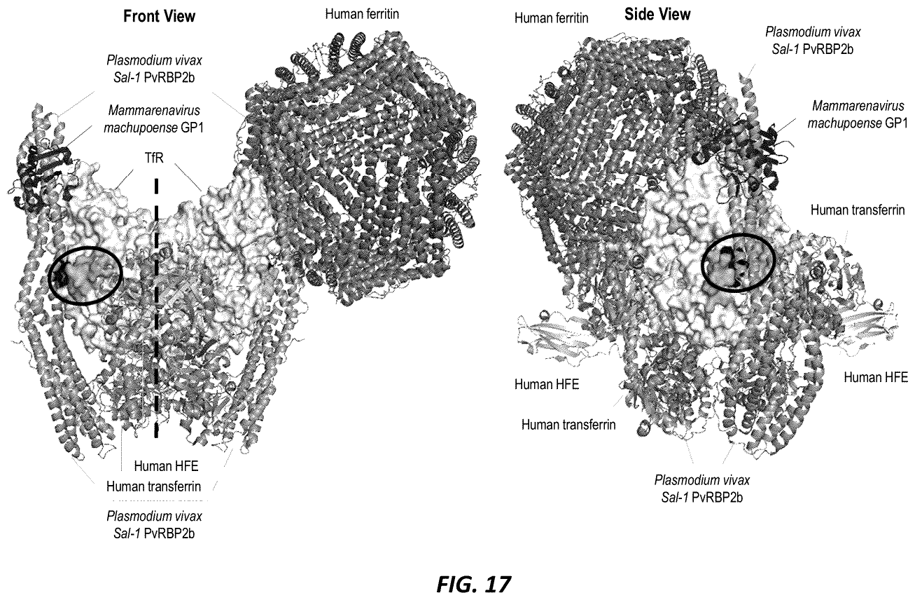

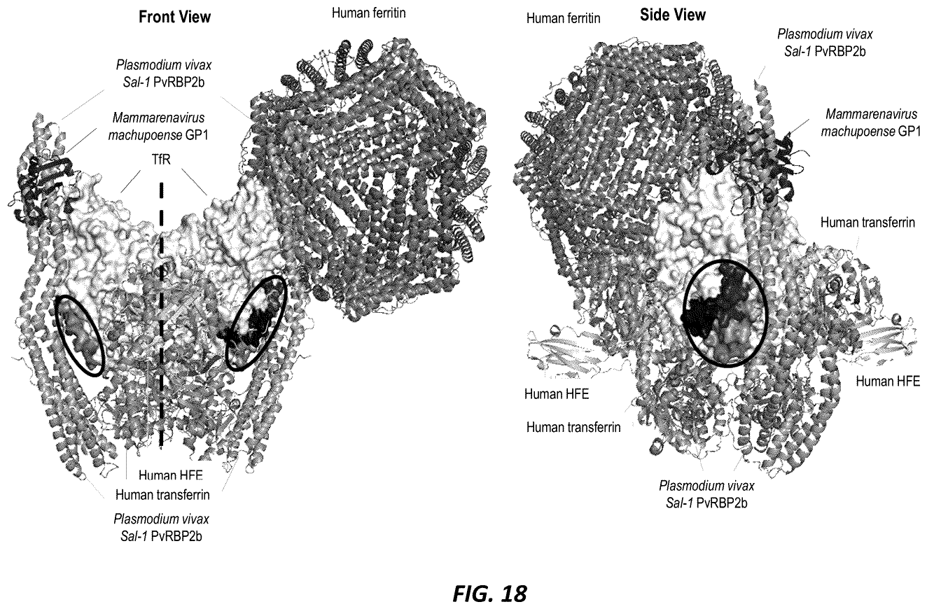

Tabulated summaries of data of the present Example are described in Tables 8-4 to Table 8-8. FIGS. 14-18 correspond to the tables below.

TABLE 8-4
Antibodies that show HDX protections
in TfR apical domain and overlap wit
Mammarenavirus machupoense GP1, canine
parvovirus, human ferritin, and plasmodium
vivax Sal-1 PvRBP2b binding sites.

| | | | m/hTfR | |
|---|---|---|---|---|
| | | | residues | |
| | | | with | |
| | | | significant | |
| | | | changes in | |
| | | | deuterium | Sequence |
| Antibody | REGN# | Antigen | % | coverage |
| H1H12841B | REGN17511 | hTfR. | 199-224 | ~92.9% |
| | | mmh | SVIIVDKNGRL | |
| | | | VYLVENPGGYV | |
| | | | AYSK | |
| | | | (SEQ ID | |
| | | | NO: 513) | |
| H1H12845B | REGN17513 | hmm. | 199-222 | ~88.1% |
| | | hTfR | SVIIVDKNGRL | |
| | | | VYLVENPGGYV | |
| | | | AY | |
| | | | (SEQ ID | |
| | | | NO: 517) | |

TABLE 8-5
Antibodies with HDX protections in TfR
apical domain that are not shared by
other TfR binding partners listed in
Table 8-4.

| | | | m/hTfR | |
|---|---|---|---|---|
| | | | residues | |
| | | | with | |
| | | | significant | |
| | | | changes | |
| | | | in | |
| | | | deuterium | Sequence |
| Antibody | REGN# | Antigen | % | coverage |
| H1H31874B | REGN17517 | hTfR. | 243-246 | ~92.9% |
| | | mmh | FEDL | |
| | | | (SEQ ID | |
| | | | NO: 519) | |
| H1H12839B | REGN17510 | hmm. | 238-246 | ~88.3% |
| | | hTfR | GTKKDFEDL | |
| | | | (SEQ ID | |
| | | | NO: 512) | |

TABLE 8-6
Antibodies with HDX protections in TfR
apical domain that share binding sites
with human ferritin and plasmodium
vivax Sal-1 PvRBP2b.

| | | | m/hTfR | |
|---|---|---|---|---|
| | | | residues | |
| | | | with | |
| | | | significant | |
| | | | changes | |
| | | | in | |
| | | | deuterium | Sequence |
| Antibody | REGN# | Antigen | % | coverage |
| H1H12848B | REGN17515 | hTfR. | 281-295 | ~92.9% |
| | | mmh | IYMDQTKFP | |
| | | | IVNAEL | |
| | | | (SEQ ID | |
| | | | NO: 506) | |
| | | | 346-365 | |
| | | | FGNMEGDCPS | |
| | | | DWKTDSTCRM | |
| | | | (SEQ ID | |
| | | | NO: 518) | |
| H1H12850B | REGN17516 | hTfR. | 146-167 LLN | ~92.9% |
| | | mmh | ENSYVPREAGS | |
| | | | QKDENLAL | |
| | | | (SEQ ID | |
| | | | NO: 505) | |
| | | | 212-232 LVE | |
| | | | NPGGYVAYSKA | |
| | | | ATVTGKL | |
| | | | (SEQ ID | |
| | | | NO: 520) | |
| | | | 281-297 | |
| | | | IYMDQTKFPI | |
| | | | VNAELSE | |
| | | | (SEQ ID | |
| | | | NO: 521) | |
| | | | 337-345 | |
| | | | ISRAAAEKL | |
| | | | (SEQ ID | |
| | | | NO: 522) | |
| | | | 366-383 | |
| | | | VTSESKNVKL | |
| | | | TVSNVLKE | |
| | | | (SEQ ID | |
| | | | NO: 523) | |
| | | | 557-572 | |
| | | | FCEDTDYPYLG | |
| | | | TTMDT | |
| | | | (SEQ ID | |
| | | | NO: 524) | |

TABLE 8-7
Antibodies with HDX protections in
TfR protease-like domain and share
binding sites with plasmodium vivax
Sal-1 PvRBP2b.

| | | | m/hTfR | |
|---|---|---|---|---|
| | | | residues | |
| | | | with | |
| | | | significant | |
| | | | changes | |
| | | | in | Se- |
| | | | deuterium | quence |
| Antibody | REGN# | Antigen | % | coverage |
| H1H12798B | REGN17507 | hmm. | 146-167 | ~87.0% |
| | | hTfR | LLNENSYVP | |
| | | | REAGSQKDE | |
| | | | NLAL | |
| | | | (SEQ ID | |
| | | | NO: 505) | |
| | | | 281-295 | |
| | | | IYMDQTKFPI | |
| | | | VNAEL | |
| | | | (SEQ ID | |
| | | | NO: 506) | |
| | | | 572-576 | |
| | | | TYKEL | |
| | | | (SEQ ID | |
| | | | NO: 507) | |
| H1H12843B | REGN17512 | hmm. | 146-164 | ~88.3% |
| | | hTfR | LLNENSYVPR | |
| | | | EAGSQKDEN | |
| | | | (SEQ ID | |
| | | | NO: 514) | |
| | | | 284-295 | |
| | | | DQTKFPI | |
| | | | VNAEL | |
| | | | (SEQ ID | |
| | | | NO: 515) | |
| | | | 572-585 | |
| | | | TYKELIE | |
| | | | RIPELNK | |
| | | | (SEQ ID | |
| | | | NO: 516) | |
| H1H12847B | REGN17514 | hmm. | 146-164 | ~88.1% |
| | | hTfR | LLNENSYVP | |
| | | | REAGSQKDE | |
| | | | N | |
| | | | (SEQ ID | |
| | | | NO: 514) | |
| | | | 572-585 | |
| | | | TYKELIE | |
| | | | RIPELNK | |
| | | | (SEQ ID | |
| | | | NO: 516) | |
| H1H12835B | REGN17509 | hTfR. | 147-165 | ~92.9% |
| | | mmh | LNENSY | |
| | | | VPREAGS | |
| | | | QKDENL | |
| | | | (SEQ ID | |
| | | | NO: 511) | |

TABLE 8-8
Antibodies with HDX protections in TfR
protease-like domain. This region is not
utilized by other TfR interacting
molecules listed in Table 8-7.

| | | | m/hTfR | |
|---|---|---|---|---|
| | | | residues | |
| | | | with | |
| | | | significant | |
| | | | changes in | |
| | | | deuterium | Sequence |
| Antibody | REGN# | Antigen | % | coverage |
| H1H12799B | REGN17508 | hmm. | 128-146 | ~88.7% |
| | | hTfR | KRKLSEK | |
| | | | LDSTDFT | |
| | | | GTIKL | |
| | | | (SEQ ID | |
| | | | NO: 508) | |
| | | | 503-522 | |
| | | | YTLIEKT | |
| | | | MQNVKHP | |
| | | | VTGQFL | |
| | | | (SEQ ID | |
| | | | NO: 509) | |
| | | | 576-592 | |
| | | | LIERIPELN | |
| | | | KVARAAAE | |
| | | | (SEQ ID | |
| | | | NO: 510) | |

### REFERENCES

1. Ehring (1999) Analytical Biochemistry 267(2):252-259
2. Engen and Smith (2001) Anal. Chem. 73:256A-265A

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants to relate to each and every individual publication, database entry (e.g., Genbank sequences or GeneID entries), patent application, or patent, each of which is clearly identified in even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

We claim:

1. An antigen-binding protein that binds specifically to transferrin receptor or an antigenic-fragment thereof or variant thereof which comprises:
(i) a HCVR that comprises the HCDR1, HCDR2 and HCDR3 of a HCVR comprising the amino acid sequence set forth in SEQ ID NO: 2; 12; 22; 32; 42; 52; 62; 72; 82; 92; 102; 112; 122; 132; 142; 152; 162; 172; 182; 192; 202; 212; 222; 232; 242; 252; 262; 272; 282; 292; 302; or 312 (or a variant thereof); and/or
(ii) a LCVR that comprises the LCDR1, LCDR2 and LCDR3 of a LCVR comprising the amino acid sequence set forth in SEQ ID NO: 7; 17; 27; 37; 47; 57; 67; 77; 87; 97; 107; 117; 127; 137; 147; 157; 167; 177; 187; 197; 207; 217; 227; 237; 247; 257; 267; 277; 287; 297; 307; or 317 (or a variant thereof);
which, optionally, is fused to a payload.

2. The antigen-binding protein of claim 1 which comprises:
(1) a HCVR comprising the HCDR1, HCDR2 and HCDR3 of a HCVR that comprises the amino acid sequence set forth in SEQ ID NO: 2 (or a variant thereof); and a LCVR comprising the LCDR1, LCDR2 and LCDR3 of a LCVR that comprises the amino acid sequence set forth in SEQ ID NO: 7 (or a variant thereof);
(2) a HCVR comprising the HCDR1, HCDR2 and HCDR3 of a HCVR that comprises the amino acid sequence set forth in SEQ ID NO: 12 (or a variant thereof); and a LCVR comprising the LCDR1, LCDR2 and LCDR3 of a LCVR that comprises the amino acid sequence set forth in SEQ ID NO: 17 (or a variant thereof);
(3) a HCVR comprising the HCDR1, HCDR2 and HCDR3 of a HCVR that comprises the amino acid sequence set forth in SEQ ID NO: 22 (or a variant thereof); and a LCVR comprising the LCDR1, LCDR2 and LCDR3 of a LCVR that comprises the amino acid sequence set forth in SEQ ID NO: 27 (or a variant thereof);
(4) a HCVR comprising the HCDR1, HCDR2 and HCDR3 of a HCVR that comprises the amino acid sequence set forth in SEQ ID NO: 32 (or a variant thereof); and a LCVR comprising the LCDR1, LCDR2 and LCDR3 of a LCVR that comprises the amino acid sequence set forth in SEQ ID NO: 37 (or a variant thereof);
(5) a HCVR comprising the HCDR1, HCDR2 and HCDR3 of a HCVR that comprises the amino acid sequence set forth in SEQ ID NO: 42 (or a variant thereof); and a LCVR comprising the LCDR1, LCDR2 and LCDR3 of a LCVR that comprises the amino acid sequence set forth in SEQ ID NO: 47 (or a variant thereof);
(6) a HCVR comprising the HCDR1, HCDR2 and HCDR3 of a HCVR that comprises the amino acid sequence set forth in SEQ ID NO: 52 (or a variant thereof); and a LCVR comprising the LCDR1, LCDR2 and LCDR3 of a LCVR that comprises the amino acid sequence set forth in SEQ ID NO: 57 (or a variant thereof);
(7) a HCVR comprising the HCDR1, HCDR2 and HCDR3 of a HCVR that comprises the amino acid sequence set forth in SEQ ID NO: 62 (or a variant thereof); and a LCVR comprising the LCDR1, LCDR2 and LCDR3 of a LCVR that comprises the amino acid sequence set forth in SEQ ID NO: 67 (or a variant thereof);
(8) a HCVR comprising the HCDR1, HCDR2 and HCDR3 of a HCVR that comprises the amino acid sequence set forth in SEQ ID NO: 72 (or a variant thereof); and a LCVR comprising the LCDR1, LCDR2 and LCDR3 of a LCVR that comprises the amino acid sequence set forth in SEQ ID NO: 77 (or a variant thereof);
(9) a HCVR comprising the HCDR1, HCDR2 and HCDR3 of a HCVR that comprises the amino acid sequence set forth in SEQ ID NO: 82 (or a variant thereof); and a LCVR comprising the LCDR1, LCDR2 and LCDR3 of a LCVR that comprises the amino acid sequence set forth in SEQ ID NO: 87 (or a variant thereof);
(10) a HCVR comprising the HCDR1, HCDR2 and HCDR3 of a HCVR that comprises the amino acid sequence set forth in SEQ ID NO: 92 (or a variant thereof); and a LCVR comprising the LCDR1, LCDR2 and LCDR3 of a LCVR that comprises the amino acid sequence set forth in SEQ ID NO: 97 (or a variant thereof);
(11) a HCVR comprising the HCDR1, HCDR2 and HCDR3 of a HCVR that comprises the amino acid sequence set forth in SEQ ID NO: 102 (or a variant thereof); and a LCVR comprising the LCDR1, LCDR2 and LCDR3 of a LCVR that comprises the amino acid sequence set forth in SEQ ID NO: 107 (or a variant thereof);
(12) a HCVR comprising the HCDR1, HCDR2 and HCDR3 of a HCVR that comprises the amino acid sequence set forth in SEQ ID NO: 112 (or a variant thereof); and a LCVR comprising the LCDR1, LCDR2 and LCDR3 of a LCVR that comprises the amino acid sequence set forth in SEQ ID NO: 117 (or a variant thereof);
(13) a HCVR comprising the HCDR1, HCDR2 and HCDR3 of a HCVR that comprises the amino acid sequence set forth in SEQ ID NO: 122 (or a variant thereof); and a LCVR comprising the LCDR1, LCDR2 and LCDR3 of a LCVR that comprises the amino acid sequence set forth in SEQ ID NO: 127 (or a variant thereof);
(14) a HCVR comprising the HCDR1, HCDR2 and HCDR3 of a HCVR that comprises the amino acid sequence set forth in SEQ ID NO: 132 (or a variant thereof); and a LCVR comprising the LCDR1, LCDR2 and LCDR3 of a LCVR that comprises the amino acid sequence set forth in SEQ ID NO: 137 (or a variant thereof);
(15) a HCVR comprising the HCDR1, HCDR2 and HCDR3 of a HCVR that comprises the amino acid sequence set forth in SEQ ID NO: 142 (or a variant thereof); and a LCVR comprising the LCDR1, LCDR2 and LCDR3 of a LCVR that comprises the amino acid sequence set forth in SEQ ID NO: 147 (or a variant thereof);
(16) a HCVR comprising the HCDR1, HCDR2 and HCDR3 of a HCVR that comprises the amino acid sequence set forth in SEQ ID NO: 152 (or a variant thereof); and a LCVR comprising the LCDR1, LCDR2 and LCDR3 of a LCVR that comprises the amino acid sequence set forth in SEQ ID NO: 157 (or a variant thereof);
(17) a HCVR comprising the HCDR1, HCDR2 and HCDR3 of a HCVR that comprises the amino acid sequence set forth in SEQ ID NO: 162 (or a variant thereof); and a LCVR comprising the LCDR1, LCDR2 and LCDR3 of a LCVR that comprises the amino acid sequence set forth in SEQ ID NO: 167 (or a variant thereof);
(18) a HCVR comprising the HCDR1, HCDR2 and HCDR3 of a HCVR that comprises the amino acid sequence set forth in SEQ ID NO: 172 (or a variant thereof); and a LCVR comprising the LCDR1, LCDR2 and LCDR3 of a LCVR that comprises the amino acid sequence set forth in SEQ ID NO: 177 (or a variant thereof);
(19) a HCVR comprising the HCDR1, HCDR2 and HCDR3 of a HCVR that comprises the amino acid sequence set forth in SEQ ID NO: 182 (or a variant thereof); and a LCVR comprising the LCDR1, LCDR2 and LCDR3 of a LCVR that comprises the amino acid sequence set forth in SEQ ID NO: 187 (or a variant thereof);
(20) a HCVR comprising the HCDR1, HCDR2 and HCDR3 of a HCVR that comprises the amino acid sequence set forth in SEQ ID NO: 192 (or a variant thereof); and a LCVR comprising the LCDR1, LCDR2 and LCDR3 of a LCVR that comprises the amino acid sequence set forth in SEQ ID NO: 197 (or a variant thereof);
(21) a HCVR comprising the HCDR1, HCDR2 and HCDR3 of a HCVR that comprises the amino acid sequence set forth in SEQ ID NO: 202 (or a variant thereof); and a LCVR comprising the LCDR1, LCDR2 and LCDR3 of a LCVR that comprises the amino acid sequence set forth in SEQ ID NO: 207 (or a variant thereof);
(22) a HCVR comprising the HCDR1, HCDR2 and HCDR3 of a HCVR that comprises the amino acid sequence set forth in SEQ ID NO: 212 (or a variant thereof); and a LCVR comprising the LCDR1, LCDR2 and LCDR3 of a LCVR that comprises the amino acid sequence set forth in SEQ ID NO: 217 (or a variant thereof);
(23) a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof);
(24) a HCVR comprising the HCDR1, HCDR2 and HCDR3 of a HCVR that comprises the amino acid sequence set forth in SEQ ID NO: 232 (or a variant thereof); and a LCVR comprising the LCDR1, LCDR2 and LCDR3 of a LCVR that comprises the amino acid sequence set forth in SEQ ID NO: 237 (or a variant thereof);
(25) a HCVR comprising the HCDR1, HCDR2 and HCDR3 of a HCVR that comprises the amino acid sequence set forth in SEQ ID NO: 242 (or a variant thereof); and a LCVR comprising the LCDR1, LCDR2 and LCDR3 of a LCVR that comprises the amino acid sequence set forth in SEQ ID NO: 247 (or a variant thereof);
(26) a HCVR comprising the HCDR1, HCDR2 and HCDR3 of a HCVR that comprises the amino acid sequence set forth in SEQ ID NO: 252 (or a variant thereof); and a LCVR comprising the LCDR1, LCDR2 and LCDR3 of a LCVR that comprises the amino acid sequence set forth in SEQ ID NO: 257 (or a variant thereof);
(27) a HCVR comprising the HCDR1, HCDR2 and HCDR3 of a HCVR that comprises the amino acid sequence set forth in SEQ ID NO: 262 (or a variant thereof); and a LCVR comprising the LCDR1, LCDR2 and LCDR3 of a LCVR that comprises the amino acid sequence set forth in SEQ ID NO: 267 (or a variant thereof);
(28) a HCVR comprising the HCDR1, HCDR2 and HCDR3 of a HCVR that comprises the amino acid sequence set forth in SEQ ID NO: 272 (or a variant thereof); and a LCVR comprising the LCDR1, LCDR2 and LCDR3 of a LCVR that comprises the amino acid sequence set forth in SEQ ID NO: 277 (or a variant thereof);
(29) a HCVR comprising the HCDR1, HCDR2 and HCDR3 of a HCVR that comprises the amino acid sequence set forth in SEQ ID NO: 282 (or a variant thereof); and a LCVR comprising the LCDR1, LCDR2 and LCDR3 of a LCVR that comprises the amino acid sequence set forth in SEQ ID NO: 287 (or a variant thereof);
(30) a HCVR comprising the HCDR1, HCDR2 and HCDR3 of a HCVR that comprises the amino acid sequence set forth in SEQ ID NO: 292 (or a variant thereof); and a LCVR comprising the LCDR1, LCDR2 and LCDR3 of a LCVR that comprises the amino acid sequence set forth in SEQ ID NO: 297 (or a variant thereof);
(31) a HCVR comprising the HCDR1, HCDR2 and HCDR3 of a HCVR that comprises the amino acid sequence set forth in SEQ ID NO: 302 (or a variant thereof); and a LCVR comprising the LCDR1, LCDR2 and LCDR3 of a LCVR that comprises the amino acid sequence set forth in SEQ ID NO: 307 (or a variant thereof); and/or
(32) a HCVR comprising the HCDR1, HCDR2 and HCDR3 of a HCVR that comprises the amino acid sequence set forth in SEQ ID NO: 312 (or a variant thereof); and a LCVR comprising the LCDR1, LCDR2 and LCDR3 of a LCVR that comprises the amino acid sequence set forth in SEQ ID NO: 317 (or a variant thereof).

3. The antigen-binding protein of claim 1 or 2 which comprises:
(1) a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof);
(2) a HCVR comprising the HCDR1, HCDR2 and HCDR3 of a HCVR that comprises the amino acid sequence set forth in SEQ ID NO: 242 (or a variant thereof); and a LCVR comprising the LCDR1, LCDR2 and LCDR3 of a LCVR that comprises the amino acid sequence set forth in SEQ ID NO: 247 (or a variant thereof);
(3) a HCVR comprising the HCDR1, HCDR2 and HCDR3 of a HCVR that comprises the amino acid sequence set forth in SEQ ID NO: 132 (or a variant thereof); and a LCVR comprising the LCDR1, LCDR2 and LCDR3 of a LCVR that comprises the amino acid sequence set forth in SEQ ID NO: 137 (or a variant thereof);
(4) a HCVR comprising the HCDR1, HCDR2 and HCDR3 of a HCVR that comprises the amino acid sequence set forth in SEQ ID NO: 172 (or a variant thereof); and a LCVR comprising the LCDR1, LCDR2 and LCDR3 of a LCVR that comprises the amino acid sequence set forth in SEQ ID NO: 177 (or a variant thereof);
(5) a HCVR comprising the HCDR1, HCDR2 and HCDR3 of a HCVR that comprises the amino acid sequence set forth in SEQ ID NO: 262 (or a variant thereof); and a LCVR comprising the LCDR1, LCDR2 and LCDR3 of a LCVR that comprises the amino acid sequence set forth in SEQ ID NO: 267 (or a variant thereof); and/or
(6) a HCVR comprising the HCDR1, HCDR2 and HCDR3 of a HCVR that comprises the amino acid sequence set forth in SEQ ID NO: 272 (or a variant thereof); and a LCVR comprising the LCDR1, LCDR2 and LCDR3 of a LCVR that comprises the amino acid sequence set forth in SEQ ID NO: 277 (or a variant thereof).

4. The antigen-binding protein of any one of claims 1-3 which comprises a HCVR comprising the HCDR1, HCDR2 and HCDR3 of a HCVR that comprises the amino acid sequence set forth in SEQ ID NO: 222 (or a variant thereof); and a LCVR comprising the LCDR1, LCDR2 and LCDR3 of a LCVR that comprises the amino acid sequence set forth in SEQ ID NO: 227 (or a variant thereof).

5. The antigen-binding protein of any one of claims 1-4 which comprises:
(a) a HCVR that comprises: an HCDR1 comprising the amino acid sequence set forth in SEQ ID NO: 3 (or a variant thereof), an HCDR2 comprising the amino acid sequence set forth in SEQ ID NO: 4 (or a variant thereof), and an HCDR3 comprising the amino acid sequence set forth in SEQ ID NO: 5 (or a variant thereof); and
a LCVR that comprises: an LCDR1 comprising the amino acid sequence set forth in SEQ ID NO: 8 (or a variant thereof), an LCDR2 comprising the amino acid sequence set forth in SEQ ID NO: 9 (or a variant thereof), and an LCDR3 comprising the amino acid sequence set forth in SEQ ID NO: 10 (or a variant thereof);
(b) a HCVR that comprises: an HCDR1 comprising the amino acid sequence set forth in SEQ ID NO: 13 (or a variant thereof), an HCDR2 comprising the amino acid sequence set forth in SEQ ID NO: 14 (or a variant thereof), and an HCDR3 comprising the amino acid sequence set forth in SEQ ID NO: 15 (or a variant thereof); and
a LCVR that comprises: an LCDR1 comprising the amino acid sequence set forth in SEQ ID NO: 18 (or a variant thereof), an LCDR2 comprising the amino acid sequence set forth in SEQ ID NO: 19 (or a variant thereof), and an LCDR3 comprising the amino acid sequence set forth in SEQ ID NO: 20 (or a variant thereof);
(c) a HCVR that comprises: an HCDR1 comprising the amino acid sequence set forth in SEQ ID NO: 23 (or a variant thereof), an HCDR2 comprising the amino acid sequence set forth in SEQ ID NO: 24 (or a variant thereof), and an HCDR3 comprising the amino acid sequence set forth in SEQ ID NO: 25 (or a variant thereof); and
a LCVR that comprises: an LCDR1 comprising the amino acid sequence set forth in SEQ ID NO: 28 (or a variant thereof), an LCDR2 comprising the amino acid sequence set forth in SEQ ID NO: 29 (or a variant thereof), and an LCDR3 comprising the amino acid sequence set forth in SEQ ID NO: 30 (or a variant thereof);
(d) a HCVR that comprises: an HCDR1 comprising the amino acid sequence set forth in SEQ ID NO: 33 (or a variant thereof), an HCDR2 comprising the amino acid sequence set forth in SEQ ID NO: 34 (or a variant thereof), and an HCDR3 comprising the amino acid sequence set forth in SEQ ID NO: 35 (or a variant thereof); and
a LCVR that comprises: an LCDR1 comprising the amino acid sequence set forth in SEQ ID NO: 38 (or a variant thereof), an LCDR2 comprising the amino acid sequence set forth in SEQ ID NO: 39 (or a variant thereof), and an LCDR3 comprising the amino acid sequence set forth in SEQ ID NO: 40 (or a variant thereof);
(e) a HCVR that comprises: an HCDR1 comprising the amino acid sequence set forth in SEQ ID NO: 43 (or a variant thereof), an HCDR2 comprising the amino acid sequence set forth in SEQ ID NO: 44 (or a variant thereof), and an HCDR3 comprising the amino acid sequence set forth in SEQ ID NO: 45 (or a variant thereof); and
a LCVR that comprises: an LCDR1 comprising the amino acid sequence set forth in SEQ ID NO: 48 (or a variant thereof), an LCDR2 comprising the amino acid sequence set forth in SEQ ID NO: 49 (or a variant thereof), and an LCDR3 comprising the amino acid sequence set forth in SEQ ID NO: 50 (or a variant thereof);
(f) a HCVR that comprises: an HCDR1 comprising the amino acid sequence set forth in SEQ ID NO: 53 (or a variant thereof), an HCDR2 comprising the amino acid sequence set forth in SEQ ID NO: 54 (or a variant thereof), and an HCDR3 comprising the amino acid sequence set forth in SEQ ID NO: 55 (or a variant thereof); and
a LCVR that comprises: an LCDR1 comprising the amino acid sequence set forth in SEQ ID NO: 58 (or a variant thereof), an LCDR2 comprising the amino acid sequence set forth in SEQ ID NO: 59 (or a variant thereof), and an LCDR3 comprising the amino acid sequence set forth in SEQ ID NO: 60 (or a variant thereof);
(g) a HCVR that comprises: an HCDR1 comprising the amino acid sequence set forth in SEQ ID NO: 63 (or a variant thereof), an HCDR2 comprising the amino acid sequence set forth in SEQ ID NO: 64 (or a variant thereof), and an HCDR3 comprising the amino acid sequence set forth in SEQ ID NO: 65 (or a variant thereof); and
a LCVR that comprises: an LCDR1 comprising the amino acid sequence set forth in SEQ ID NO: 68 (or a variant thereof), an LCDR2 comprising the amino acid sequence set forth in SEQ ID NO: 69 (or a variant thereof), and an LCDR3 comprising the amino acid sequence set forth in SEQ ID NO: 70 (or a variant thereof);
(h) a HCVR that comprises: an HCDR1 comprising the amino acid sequence set forth in SEQ ID NO: 73 (or a variant thereof), an HCDR2 comprising the amino acid sequence set forth in SEQ ID NO: 74 (or a variant thereof), and an HCDR3 comprising the amino acid sequence set forth in SEQ ID NO: 75 (or a variant thereof); and
a LCVR that comprises: an LCDR1 comprising the amino acid sequence set forth in SEQ ID NO: 78 (or a variant thereof), an LCDR2 comprising the amino acid sequence set forth in SEQ ID NO: 79 (or a variant thereof), and an LCDR3 comprising the amino acid sequence set forth in SEQ ID NO: 80 (or a variant thereof);
(i) a HCVR that comprises: an HCDR1 comprising the amino acid sequence set forth in SEQ ID NO: 83 (or a variant thereof), an HCDR2 comprising the amino acid sequence set forth in SEQ ID NO: 84 (or a variant thereof), and an HCDR3 comprising the amino acid sequence set forth in SEQ ID NO: 85 (or a variant thereof); and
a LCVR that comprises: an LCDR1 comprising the amino acid sequence set forth in SEQ ID NO: 88 (or a variant thereof), an LCDR2 comprising the amino acid sequence set forth in SEQ ID NO: 89 (or a variant thereof), and an LCDR3 comprising the amino acid sequence set forth in SEQ ID NO: 90 (or a variant thereof);
(j) a HCVR that comprises: an HCDR1 comprising the amino acid sequence set forth in SEQ ID NO: 93 (or a variant thereof), an HCDR2 comprising the amino acid sequence set forth in SEQ ID NO: 94 (or a variant thereof), and an HCDR3 comprising the amino acid sequence set forth in SEQ ID NO: 95 (or a variant thereof); and
a LCVR that comprises: an LCDR1 comprising the amino acid sequence set forth in SEQ ID NO: 98 (or a variant thereof), an LCDR2 comprising the amino acid sequence set forth in SEQ ID NO: 99 (or a variant thereof), and an LCDR3 comprising the amino acid sequence set forth in SEQ ID NO: 100 (or a variant thereof);
(k) a HCVR that comprises: an HCDR1 comprising the amino acid sequence set forth in SEQ ID NO: 103 (or a variant thereof), an HCDR2 comprising the amino acid sequence set forth in SEQ ID NO: 104 (or a variant thereof), and an HCDR3 comprising the amino acid sequence set forth in SEQ ID NO: 105 (or a variant thereof); and
a LCVR that comprises: an LCDR1 comprising the amino acid sequence set forth in SEQ ID NO: 108 (or a variant thereof), an LCDR2 comprising the amino acid sequence set forth in SEQ ID NO: 109 (or a variant thereof), and an LCDR3 comprising the amino acid sequence set forth in SEQ ID NO: 110 (or a variant thereof);
(l) a HCVR that comprises: an HCDR1 comprising the amino acid sequence set forth in SEQ ID NO: 113 (or a variant thereof), an HCDR2 comprising the amino acid sequence set forth in SEQ ID NO: 114 (or a variant thereof), and an HCDR3 comprising the amino acid sequence set forth in SEQ ID NO: 115 (or a variant thereof); and
a LCVR that comprises: an LCDR1 comprising the amino acid sequence set forth in SEQ ID NO: 118 (or a variant thereof), an LCDR2 comprising the amino acid sequence set forth in SEQ ID NO: 119 (or a variant thereof), and an LCDR3 comprising the amino acid sequence set forth in SEQ ID NO: 120 (or a variant thereof);
(m) a HCVR that comprises: an HCDR1 comprising the amino acid sequence set forth in SEQ ID NO: 123 (or a variant thereof), an HCDR2 comprising the amino acid sequence set forth in SEQ ID NO: 124 (or a variant thereof), and an HCDR3 comprising the amino acid sequence set forth in SEQ ID NO: 125 (or a variant thereof); and
a LCVR that comprises: an LCDR1 comprising the amino acid sequence set forth in SEQ ID NO: 128 (or a variant thereof), an LCDR2 comprising the amino acid sequence set forth in SEQ ID NO: 129 (or a variant thereof), and an LCDR3 comprising the amino acid sequence set forth in SEQ ID NO: 130 (or a variant thereof);
(n) a HCVR that comprises: an HCDR1 comprising the amino acid sequence set forth in SEQ ID NO: 133 (or a variant thereof), an HCDR2 comprising the amino acid sequence set forth in SEQ ID NO: 134 (or a variant thereof), and an HCDR3 comprising the amino acid sequence set forth in SEQ ID NO: 135 (or a variant thereof); and
a LCVR that comprises: an LCDR1 comprising the amino acid sequence set forth in SEQ ID NO: 138 (or a variant thereof), an LCDR2 comprising the amino acid sequence set forth in SEQ ID NO: 139 (or a variant thereof), and an LCDR3 comprising the amino acid sequence set forth in SEQ ID NO: 140 (or a variant thereof);
(o) a HCVR that comprises: an HCDR1 comprising the amino acid sequence set forth in SEQ ID NO: 143 (or a variant thereof), an HCDR2 comprising the amino acid sequence set forth in SEQ ID NO: 144 (or a variant thereof), and an HCDR3 comprising the amino acid sequence set forth in SEQ ID NO: 145 (or a variant thereof); and
a LCVR that comprises: an LCDR1 comprising the amino acid sequence set forth in SEQ ID NO: 148 (or a variant thereof), an LCDR2 comprising the amino acid sequence set forth in SEQ ID NO: 149 (or a variant thereof), and an LCDR3 comprising the amino acid sequence set forth in SEQ ID NO: 150 (or a variant thereof);
(p) a HCVR that comprises: an HCDR1 comprising the amino acid sequence set forth in SEQ ID NO: 153 (or a variant thereof), an HCDR2 comprising the amino acid sequence set forth in SEQ ID NO: 154 (or a variant thereof), and an HCDR3 comprising the amino acid sequence set forth in SEQ ID NO: 155 (or a variant thereof); and
a LCVR that comprises: an LCDR1 comprising the amino acid sequence set forth in SEQ ID NO: 158 (or a variant thereof), an LCDR2 comprising the amino acid sequence set forth in SEQ ID NO: 159 (or a variant thereof), and an LCDR3 comprising the amino acid sequence set forth in SEQ ID NO: 160 (or a variant thereof);
(q) a HCVR that comprises: an HCDR1 comprising the amino acid sequence set forth in SEQ ID NO: 163 (or a variant thereof), an HCDR2 comprising the amino acid sequence set forth in SEQ ID NO: 164 (or a variant thereof), and an HCDR3 comprising the amino acid sequence set forth in SEQ ID NO: 165 (or a variant thereof); and
a LCVR that comprises: an LCDR1 comprising the amino acid sequence set forth in SEQ ID NO: 168 (or a variant thereof), an LCDR2 comprising the amino acid sequence set forth in SEQ ID NO: 169 (or a variant thereof), and an LCDR3 comprising the amino acid sequence set forth in SEQ ID NO: 170 (or a variant thereof);
(r) a HCVR that comprises: an HCDR1 comprising the amino acid sequence set forth in SEQ ID NO: 173 (or a variant thereof), an HCDR2 comprising the amino acid sequence set forth in SEQ ID NO: 174 (or a variant thereof), and an HCDR3 comprising the amino acid sequence set forth in SEQ ID NO: 175 (or a variant thereof); and
a LCVR that comprises: an LCDR1 comprising the amino acid sequence set forth in SEQ ID NO: 178 (or a variant thereof), an LCDR2 comprising the amino acid sequence set forth in SEQ ID NO: 179 (or a variant thereof), and an LCDR3 comprising the amino acid sequence set forth in SEQ ID NO: 180 (or a variant thereof);
(s) a HCVR that comprises: an HCDR1 comprising the amino acid sequence set forth in SEQ ID NO: 183 (or a variant thereof), an HCDR2 comprising the amino acid sequence set forth in SEQ ID NO: 184 (or a variant thereof), and an HCDR3 comprising the amino acid sequence set forth in SEQ ID NO: 185 (or a variant thereof); and
a LCVR that comprises: an LCDR1 comprising the amino acid sequence set forth in SEQ ID NO: 188 (or a variant thereof), an LCDR2 comprising the amino acid sequence set forth in SEQ ID NO: 189 (or a variant thereof), and an LCDR3 comprising the amino acid sequence set forth in SEQ ID NO: 190 (or a variant thereof);
(t) a HCVR that comprises: an HCDR1 comprising the amino acid sequence set forth in SEQ ID NO: 193 (or a variant thereof), an HCDR2 comprising the amino acid sequence set forth in SEQ ID NO: 194 (or a variant thereof), and an HCDR3 comprising the amino acid sequence set forth in SEQ ID NO: 195 (or a variant thereof); and
a LCVR that comprises: an LCDR1 comprising the amino acid sequence set forth in SEQ ID NO: 198 (or a variant thereof), an LCDR2 comprising the amino acid sequence set forth in SEQ ID NO: 199 (or a variant thereof), and an LCDR3 comprising the amino acid sequence set forth in SEQ ID NO: 200 (or a variant thereof);
(u) a HCVR that comprises: an HCDR1 comprising the amino acid sequence set forth in SEQ ID NO: 203 (or a variant thereof), an HCDR2 comprising the amino acid sequence set forth in SEQ ID NO: 204 (or a variant thereof), and an HCDR3 comprising the amino acid sequence set forth in SEQ ID NO: 205 (or a variant thereof); and
a LCVR that comprises: an LCDR1 comprising the amino acid sequence set forth in SEQ ID NO: 208 (or a variant thereof), an LCDR2 comprising the amino acid sequence set forth in SEQ ID NO: 209 (or a variant thereof), and an LCDR3 comprising the amino acid sequence set forth in SEQ ID NO: 210 (or a variant thereof);
(v) a HCVR that comprises: an HCDR1 comprising the amino acid sequence set forth in SEQ ID NO: 213 (or a variant thereof), an HCDR2 comprising the amino acid sequence set forth in SEQ ID NO: 214 (or a variant thereof), and an HCDR3 comprising the amino acid sequence set forth in SEQ ID NO: 215 (or a variant thereof); and
a LCVR that comprises: an LCDR1 comprising the amino acid sequence set forth in SEQ ID NO: 218 (or a variant thereof), an LCDR2 comprising the amino acid sequence set forth in SEQ ID NO: 219 (or a variant thereof), and an LCDR3 comprising the amino acid sequence set forth in SEQ ID NO: 220 (or a variant thereof);
(w) a HCVR that comprises: an HCDR1 comprising the amino acid sequence set forth in SEQ ID NO: 223 (or a variant thereof), an HCDR2 comprising the amino acid sequence set forth in SEQ ID NO: 224 (or a variant thereof), and an HCDR3 comprising the amino acid sequence set forth in SEQ ID NO: 225 (or a variant thereof); and
a LCVR that comprises: an LCDR1 comprising the amino acid sequence set forth in SEQ ID NO: 228 (or a variant thereof), an LCDR2 comprising the amino acid sequence set forth in SEQ ID NO: 229 (or a variant thereof), and an LCDR3 comprising the amino acid sequence set forth in SEQ ID NO: 230 (or a variant thereof);
(x) a HCVR that comprises: an HCDR1 comprising the amino acid sequence set forth in SEQ ID NO: 233 (or a variant thereof), an HCDR2 comprising the amino acid sequence set forth in SEQ ID NO: 234 (or a variant thereof), and an HCDR3 comprising the amino acid sequence set forth in SEQ ID NO: 235 (or a variant thereof); and
a LCVR that comprises: an LCDR1 comprising the amino acid sequence set forth in SEQ ID NO: 238 (or a variant thereof), an LCDR2 comprising the amino acid sequence set forth in SEQ ID NO: 239 (or a variant thereof), and an LCDR3 comprising the amino acid sequence set forth in SEQ ID NO: 240 (or a variant thereof);
(y) a HCVR that comprises: an HCDR1 comprising the amino acid sequence set forth in SEQ ID NO: 243 (or a variant thereof), an HCDR2 comprising the amino acid sequence set forth in SEQ ID NO: 244 (or a variant thereof), and an HCDR3 comprising the amino acid sequence set forth in SEQ ID NO: 245 (or a variant thereof); and
a LCVR that comprises: an LCDR1 comprising the amino acid sequence set forth in SEQ ID NO: 248 (or a variant thereof), an LCDR2 comprising the amino acid sequence set forth in SEQ ID NO: 249 (or a variant thereof), and an LCDR3 comprising the amino acid sequence set forth in SEQ ID NO: 250 (or a variant thereof);
(z) a HCVR that comprises: an HCDR1 comprising the amino acid sequence set forth in SEQ ID NO: 253 (or a variant thereof), an HCDR2 comprising the amino acid sequence set forth in SEQ ID NO: 254 (or a variant thereof), and an HCDR3 comprising the amino acid sequence set forth in SEQ ID NO: 255 (or a variant thereof); and
a LCVR that comprises: an LCDR1 comprising the amino acid sequence set forth in SEQ ID NO: 258 (or a variant thereof), an LCDR2 comprising the amino acid sequence set forth in SEQ ID NO: 259 (or a variant thereof), and an LCDR3 comprising the amino acid sequence set forth in SEQ ID NO: 260 (or a variant thereof);
(aa) a HCVR that comprises: an HCDR1 comprising the amino acid sequence set forth in SEQ ID NO: 263 (or a variant thereof), an HCDR2 comprising the amino acid sequence set forth in SEQ ID NO: 264 (or a variant thereof), and an HCDR3 comprising the amino acid sequence set forth in SEQ ID NO: 265 (or a variant thereof); and
a LCVR that comprises: an LCDR1 comprising the amino acid sequence set forth in SEQ ID NO: 268 (or a variant thereof), an LCDR2 comprising the amino acid sequence set forth in SEQ ID NO: 269 (or a variant thereof), and an LCDR3 comprising the amino acid sequence set forth in SEQ ID NO: 270 (or a variant thereof);
(ab) a HCVR that comprises: an HCDR1 comprising the amino acid sequence set forth in SEQ ID NO: 273 (or a variant thereof), an HCDR2 comprising the amino acid sequence set forth in SEQ ID NO: 274 (or a variant thereof), and an HCDR3 comprising the amino acid sequence set forth in SEQ ID NO: 275 (or a variant thereof); and
a LCVR that comprises: an LCDR1 comprising the amino acid sequence set forth in SEQ ID NO: 278 (or a variant thereof), an LCDR2 comprising the amino acid sequence set forth in SEQ ID NO: 279 (or a variant thereof), and an LCDR3 comprising the amino acid sequence set forth in SEQ ID NO: 280 (or a variant thereof);
(ac) a HCVR that comprises: an HCDR1 comprising the amino acid sequence set forth in SEQ ID NO: 283 (or a variant thereof), an HCDR2 comprising the amino acid sequence set forth in SEQ ID NO: 284 (or a variant thereof), and an HCDR3 comprising the amino acid sequence set forth in SEQ ID NO: 285 (or a variant thereof); and
a LCVR that comprises: an LCDR1 comprising the amino acid sequence set forth in SEQ ID NO: 288 (or a variant thereof), an LCDR2 comprising the amino acid sequence set forth in SEQ ID NO: 289 (or a variant thereof), and an LCDR3 comprising the amino acid sequence set forth in SEQ ID NO: 290 (or a variant thereof);
(ad) a HCVR that comprises: an HCDR1 comprising the amino acid sequence set forth in SEQ ID NO: 293 (or a variant thereof), an HCDR2 comprising the amino acid sequence set forth in SEQ ID NO: 294 (or a variant thereof), and an HCDR3 comprising the amino acid sequence set forth in SEQ ID NO: 295 (or a variant thereof); and
a LCVR that comprises: an LCDR1 comprising the amino acid sequence set forth in SEQ ID NO: 298 (or a variant thereof), an LCDR2 comprising the amino acid sequence set forth in SEQ ID NO: 299 (or a variant thereof), and an LCDR3 comprising the amino acid sequence set forth in SEQ ID NO: 300 (or a variant thereof);
(ae) a HCVR that comprises: an HCDR1 comprising the amino acid sequence set forth in SEQ ID NO: 303 (or a variant thereof), an HCDR2 comprising the amino acid sequence set forth in SEQ ID NO: 304 (or a variant thereof), and an HCDR3 comprising the amino acid sequence set forth in SEQ ID NO: 305 (or a variant thereof); and
a LCVR that comprises: an LCDR1 comprising the amino acid sequence set forth in SEQ ID NO: 308 (or a variant thereof), an LCDR2 comprising the amino acid sequence set forth in SEQ ID NO: 309 (or a variant thereof), and an LCDR3 comprising the amino acid sequence set forth in SEQ ID NO: 310 (or a variant thereof); and/or
(af) a HCVR that comprises: an HCDR1 comprising the amino acid sequence set forth in SEQ ID NO: 313 (or a variant thereof), an HCDR2 comprising the amino acid sequence set forth in SEQ ID NO: 314 (or a variant thereof), and an HCDR3 comprising the amino acid sequence set forth in SEQ ID NO: 315 (or a variant thereof); and
a LCVR that comprises: an LCDR1 comprising the amino acid sequence set forth in SEQ ID NO: 318 (or a variant thereof), an LCDR2 comprising the amino acid sequence set forth in SEQ ID NO: 319 (or a variant thereof), and an LCDR3 comprising the amino acid sequence set forth in SEQ ID NO: 320 (or a variant thereof).

6. The antigen-binding protein of any one of claims 1-5 which comprises:
(a) a HCVR that comprises: an HCDR1 comprising the amino acid sequence set forth in SEQ ID NO: 223 (or a variant thereof), an HCDR2 comprising the amino acid sequence set forth in SEQ ID NO: 224 (or a variant thereof), and an HCDR3 comprising the amino acid sequence set forth in SEQ ID NO: 225 (or a variant thereof); and
a LCVR that comprises: an LCDR1 comprising the amino acid sequence set forth in SEQ ID NO: 228 (or a variant thereof), an LCDR2 comprising the amino acid sequence set forth in SEQ ID NO: 229 (or a variant thereof), and an LCDR3 comprising the amino acid sequence set forth in SEQ ID NO: 230 (or a variant thereof);
(b) a HCVR that comprises: an HCDR1 comprising the amino acid sequence set forth in SEQ ID NO: 243 (or a variant thereof), an HCDR2 comprising the amino acid sequence set forth in SEQ ID NO: 244 (or a variant thereof), and an HCDR3 comprising the amino acid sequence set forth in SEQ ID NO: 245 (or a variant thereof); and
a LCVR that comprises: an LCDR1 comprising the amino acid sequence set forth in SEQ ID NO: 248 (or a variant thereof), an LCDR2 comprising the amino acid sequence set forth in SEQ ID NO: 249 (or a variant thereof), and an LCDR3 comprising the amino acid sequence set forth in SEQ ID NO: 250 (or a variant thereof);
(c) a HCVR that comprises: an HCDR1 comprising the amino acid sequence set forth in SEQ ID NO: 133 (or a variant thereof), an HCDR2 comprising the amino acid sequence set forth in SEQ ID NO: 134 (or a variant thereof), and an HCDR3 comprising the amino acid sequence set forth in SEQ ID NO: 135 (or a variant thereof); and
a LCVR that comprises: an LCDR1 comprising the amino acid sequence set forth in SEQ ID NO: 138 (or a variant thereof), an LCDR2 comprising the amino acid sequence set forth in SEQ ID NO: 139 (or a variant thereof), and an LCDR3 comprising the amino acid sequence set forth in SEQ ID NO: 140 (or a variant thereof);
(d) a HCVR that comprises: an HCDR1 comprising the amino acid sequence set forth in SEQ ID NO: 173 (or a variant thereof), an HCDR2 comprising the amino acid sequence set forth in SEQ ID NO: 174 (or a variant thereof), and an HCDR3 comprising the amino acid sequence set forth in SEQ ID NO: 175 (or a variant thereof); and
a LCVR that comprises: an LCDR1 comprising the amino acid sequence set forth in SEQ ID NO: 178 (or a variant thereof), an LCDR2 comprising the amino acid sequence set forth in SEQ ID NO: 179 (or a variant thereof), and an LCDR3 comprising the amino acid sequence set forth in SEQ ID NO: 180 (or a variant thereof);
(e) a HCVR that comprises: an HCDR1 comprising the amino acid sequence set forth in SEQ ID NO: 263 (or a variant thereof), an HCDR2 comprising the amino acid sequence set forth in SEQ ID NO: 264 (or a variant thereof), and an HCDR3 comprising the amino acid sequence set forth in SEQ ID NO: 265 (or a variant thereof); and
a LCVR that comprises: an LCDR1 comprising the amino acid sequence set forth in SEQ ID NO: 268 (or a variant thereof), an LCDR2 comprising the amino acid sequence set forth in SEQ ID NO: 269 (or a variant thereof), and an LCDR3 comprising the amino acid sequence set forth in SEQ ID NO: 270 (or a variant thereof); and/or
(f) a HCVR that comprises: an HCDR1 comprising the amino acid sequence set forth in SEQ ID NO: 273 (or a variant thereof), an HCDR2 comprising the amino acid sequence set forth in SEQ ID NO: 274 (or a variant thereof), and an HCDR3 comprising the amino acid sequence set forth in SEQ ID NO: 275 (or a variant thereof); and
a LCVR that comprises: an LCDR1 comprising the amino acid sequence set forth in SEQ ID NO: 278 (or a variant thereof), an LCDR2 comprising the amino acid sequence set forth in SEQ ID NO: 279 (or a variant thereof), and an LCDR3 comprising the amino acid sequence set forth in SEQ ID NO: 280 (or a variant thereof).

7. The antigen-binding protein of any one of claims 1-6 which comprises:
a HCVR that comprises: an HCDR1 comprising the amino acid sequence set forth in SEQ ID NO: 223 (or a variant thereof), an HCDR2 comprising the amino acid sequence set forth in SEQ ID NO: 224 (or a variant thereof), and an HCDR3 comprising the amino acid sequence set forth in SEQ ID NO: 225 (or a variant thereof); and
a LCVR that comprises: an LCDR1 comprising the amino acid sequence set forth in SEQ ID NO: 228 (or a variant thereof), an LCDR2 comprising the amino acid sequence set forth in SEQ ID NO: 229 (or a variant thereof), and an LCDR3 comprising the amino acid sequence set forth in SEQ ID NO: 230 (or a variant thereof).

8. The antigen-binding protein of any one of claims 1-7 which comprises:
(i) a HCVR that comprises the amino acid sequence set forth in SEQ ID NO: 2 (or a variant thereof); and a LCVR that comprises the amino acid sequence set forth in SEQ ID NO: 7 (or a variant thereof);
(ii) a HCVR that comprises the amino acid sequence set forth in SEQ ID NO: 12 (or a variant thereof); and a LCVR that comprises the amino acid sequence set forth in SEQ ID NO: 17 (or a variant thereof);
(iii) a HCVR that comprises the amino acid sequence set forth in SEQ ID NO: 22 (or a variant thereof); and a LCVR that comprises the amino acid sequence set forth in SEQ ID NO: 27 (or a variant thereof);
(iv) a HCVR that comprises the amino acid sequence set forth in SEQ ID NO: 32 (or a variant thereof); and a LCVR that comprises the amino acid sequence set forth in SEQ ID NO: 37 (or a variant thereof);
(v) a HCVR that comprises the amino acid sequence set forth in SEQ ID NO: 42 (or a variant thereof); and a LCVR that comprises the amino acid sequence set forth in SEQ ID NO: 47 (or a variant thereof);
(vi) a HCVR that comprises the amino acid sequence set forth in SEQ ID NO: 52 (or a variant thereof); and a LCVR that comprises the amino acid sequence set forth in SEQ ID NO: 57 (or a variant thereof);
(vii) a HCVR that comprises the amino acid sequence set forth in SEQ ID NO: 62 (or a variant thereof); and a LCVR that comprises the amino acid sequence set forth in SEQ ID NO: 67 (or a variant thereof);
(viii) a HCVR that comprises the amino acid sequence set forth in SEQ ID NO: 72 (or a variant thereof); and a LCVR that comprises the amino acid sequence set forth in SEQ ID NO: 77 (or a variant thereof);
(ix) a HCVR that comprises the amino acid sequence set forth in SEQ ID NO: 82 (or a variant thereof); and a LCVR that comprises the amino acid sequence set forth in SEQ ID NO: 87 (or a variant thereof);
(x) a HCVR that comprises the amino acid sequence set forth in SEQ ID NO: 92 (or a variant thereof); and a LCVR that comprises the amino acid sequence set forth in SEQ ID NO: 97 (or a variant thereof);
(xi) a HCVR that comprises the amino acid sequence set forth in SEQ ID NO: 102 (or a variant thereof); and a LCVR that comprises the amino acid sequence set forth in SEQ ID NO: 107 (or a variant thereof);
(xii) a HCVR that comprises the amino acid sequence set forth in SEQ ID NO: 112 (or a variant thereof); and a LCVR that comprises the amino acid sequence set forth in SEQ ID NO: 117 (or a variant thereof);
(xiii) a HCVR that comprises the amino acid sequence set forth in SEQ ID NO: 122 (or a variant thereof); and a LCVR that comprises the amino acid sequence set forth in SEQ ID NO: 127 (or a variant thereof);
(xiv) a HCVR that comprises the amino acid sequence set forth in SEQ ID NO: 132 (or a variant thereof); and a LCVR that comprises the amino acid sequence set forth in SEQ ID NO: 137 (or a variant thereof);
(xv) a HCVR that comprises the amino acid sequence set forth in SEQ ID NO: 142 (or a variant thereof); and a LCVR that comprises the amino acid sequence set forth in SEQ ID NO: 147 (or a variant thereof);
(xvi) a HCVR that comprises the amino acid sequence set forth in SEQ ID NO: 152 (or a variant thereof); and a LCVR that comprises the amino acid sequence set forth in SEQ ID NO: 157 (or a variant thereof);
(xvii) a HCVR that comprises the amino acid sequence set forth in SEQ ID NO: 162 (or a variant thereof); and a LCVR that comprises the amino acid sequence set forth in SEQ ID NO: 167 (or a variant thereof);
(xviii) a HCVR that comprises the amino acid sequence set forth in SEQ ID NO: 172 (or a variant thereof); and a LCVR that comprises the amino acid sequence set forth in SEQ ID NO: 177 (or a variant thereof);
(xix) a HCVR that comprises the amino acid sequence set forth in SEQ ID NO: 182 (or a variant thereof); and a LCVR that comprises the amino acid sequence set forth in SEQ ID NO: 187 (or a variant thereof);
(xx) a HCVR that comprises the amino acid sequence set forth in SEQ ID NO: 192 (or a variant thereof); and a LCVR that comprises the amino acid sequence set forth in SEQ ID NO: 197 (or a variant thereof);
(xxi) a HCVR that comprises the amino acid sequence set forth in SEQ ID NO: 202 (or a variant thereof); and a LCVR that comprises the amino acid sequence set forth in SEQ ID NO: 207 (or a variant thereof);
(xxii) a HCVR that comprises the amino acid sequence set forth in SEQ ID NO: 212 (or a variant thereof); and a LCVR that comprises the amino acid sequence set forth in SEQ ID NO: 217 (or a variant thereof);
(xxiii) a HCVR that comprises the amino acid sequence set forth in SEQ ID NO: 222 (or a variant thereof); and a LCVR that comprises the amino acid sequence set forth in SEQ ID NO: 227 (or a variant thereof);
(xxiv) a HCVR that comprises the amino acid sequence set forth in SEQ ID NO: 232 (or a variant thereof); and a LCVR that comprises the amino acid sequence set forth in SEQ ID NO: 237 (or a variant thereof);
(xxv) a HCVR that comprises the amino acid sequence set forth in SEQ ID NO: 242 (or a variant thereof); and a LCVR that comprises the amino acid sequence set forth in SEQ ID NO: 247 (or a variant thereof);
(xxvi) a HCVR that comprises the amino acid sequence set forth in SEQ ID NO: 252 (or a variant thereof); and a LCVR that comprises the amino acid sequence set forth in SEQ ID NO: 257 (or a variant thereof);
(xxvii) a HCVR that comprises the amino acid sequence set forth in SEQ ID NO: 262 (or a variant thereof); and a LCVR that comprises the amino acid sequence set forth in SEQ ID NO: 267 (or a variant thereof);
(xxviii) a HCVR that comprises the amino acid sequence set forth in SEQ ID NO: 272 (or a variant thereof); and a LCVR that comprises the amino acid sequence set forth in SEQ ID NO: 277 (or a variant thereof);
(xxix) a HCVR that comprises the amino acid sequence set forth in SEQ ID NO: 282 (or a variant thereof); and a LCVR that comprises the amino acid sequence set forth in SEQ ID NO: 287 (or a variant thereof);
(xxx) a HCVR that comprises the amino acid sequence set forth in SEQ ID NO: 292 (or a variant thereof); and a LCVR that comprises the amino acid sequence set forth in SEQ ID NO: 297 (or a variant thereof);
(xxxi) a HCVR that comprises the amino acid sequence set forth in SEQ ID NO: 302 (or a variant thereof); and a LCVR that comprises the amino acid sequence set forth in SEQ ID NO: 307 (or a variant thereof); and/or
(xxxii) a HCVR that comprises the amino acid sequence set forth in SEQ ID NO: 312 (or a variant thereof); and a LCVR that comprises the amino acid sequence set forth in SEQ ID NO: 317 (or a variant thereof).

9. The antigen-binding protein of any one of claims 1-8 which comprises:
(i) a HCVR that comprises the amino acid sequence set forth in SEQ ID NO: 222 (or a variant thereof); and a LCVR that comprises the amino acid sequence set forth in SEQ ID NO: 227 (or a variant thereof);
(ii) a HCVR that comprises the amino acid sequence set forth in SEQ ID NO: 242 (or a variant thereof); and a LCVR that comprises the amino acid sequence set forth in SEQ ID NO: 247 (or a variant thereof);
(iii) a HCVR that comprises the amino acid sequence set forth in SEQ ID NO: 132 (or a variant thereof); and a LCVR that comprises the amino acid sequence set forth in SEQ ID NO: 137 (or a variant thereof);
(iv) a HCVR that comprises the amino acid sequence set forth in SEQ ID NO: 172 (or a variant thereof); and a LCVR that comprises the amino acid sequence set forth in SEQ ID NO: 177 (or a variant thereof);
(v) a HCVR that comprises the amino acid sequence set forth in SEQ ID NO: 262 (or a variant thereof); and a LCVR that comprises the amino acid sequence set forth in SEQ ID NO: 267 (or a variant thereof); and/or
(vi) a HCVR that comprises the amino acid sequence set forth in SEQ ID NO: 272 (or a variant thereof); and a LCVR that comprises the amino acid sequence set forth in SEQ ID NO: 277 (or a variant thereof).

10. The antigen-binding protein of any one of claims 1-9 which comprises a HCVR that comprises the amino acid sequence set forth in SEQ ID NO: 222 (or a variant thereof); and a LCVR that comprises the amino acid sequence set forth in SEQ ID NO: 227 (or a variant thereof).

11. The antigen-binding protein of any one of claims 1-10, wherein the transferrin receptor is the human transferrin receptor or a variant thereof.

12. The antigen-binding protein of any one of claims 1-11 which is an antibody or antigen-binding fragment thereof.

13. The antigen-binding protein of any one of claims 1-12 which is a Fab.

14. The antigen-binding protein of any one of claims 1-12 which is an scFv;
optionally wherein the scFv and the payload are connected by a peptide linker which is -(GGGGS)ₘ- (SEQ ID NO: 426); wherein m is 1, 2, 3, 4, 5, 6, 7, 8, 9 or 10; and
optionally, wherein the scFv variable regions are connected by a peptide linker which is -(GGGGS)ₙ- (SEQ ID NO: 426); wherein n is 1, 2, 3, 4, 5, 6, 7, 8, 9 or 10.

15. The antigen-binding protein of claim 14, wherein the scFv comprises the amino acid sequence set forth in SEQ ID NO: 443 (or a variant thereof), comprises the amino acid sequence set forth in SEQ ID NO: 440 (or a variant thereof), comprises the amino acid sequence set forth in SEQ ID NO: 429 (or a variant thereof), comprises the amino acid sequence set forth in SEQ ID NO: 433 (or a variant thereof), comprises the amino acid sequence set forth in SEQ ID NO: 442 (or a variant thereof), or comprises the amino acid sequence set forth in SEQ ID NO: 438 (or a variant thereof).

16. The antigen-binding protein of claim 14 or 15, wherein the scFv comprises the amino acid sequence set forth in SEQ ID NO: 443 (or a variant thereof).

17. An antigen-binding protein that binds specifically to transferrin receptor or an antigenic-fragment thereof or variant thereof which binds to one or more epitopes of hTfR selected from:
(a) an epitope comprising the sequence LLNE (SEQ ID NO: 525) and/or an epitope comprising the sequence TYKEL (SEQ ID NO: 507);
(b) an epitope comprising the sequence DSTDFTGT (SEQ ID NO: 526) and/or an epitope comprising the sequence VKHPVTGQF (SEQ ID NO: 527) and/or an epitope comprising the sequence IERIPEL (SEQ ID NO: 528);
(c) an epitope comprising the sequence LNENSYVPREAGSQKDEN (SEQ ID NO: 529);
(d) an epitope comprising the sequence FEDL (SEQ ID NO: 519);
(e) an epitope comprising the sequence IVDKNGRL (SEQ ID NO: 530);
(f) an epitope comprising the sequence IVDKNGRLVY (SEQ ID NO: 531);
(g) an epitope comprising the sequence DQTKF (SEQ ID NO: 532);
(h) an epitope comprising the sequence LVENPGGY (SEQ ID NO: 533) and/or an epitope comprising the sequence PIVNAELSF (SEQ ID NO: 534) and/or an epitope comprising the sequence PYLGTTMDT (SEQ ID NO: 535);
(i) an epitope comprising the sequence LLNENSYVPREAGSQKDENLAL (SEQ ID NO: 505) and/or an epitope comprising the sequence IYMDQTKFPIVNAEL (SEQ ID NO: 506) and/or an epitope comprising the sequence TYKEL (SEQ ID NO: 507);
(j) an epitope comprising the sequence KRKLSEKLDSTDFTGTIKL (SEQ ID NO: 508) and/or an epitope comprising the sequence YTLIEKTMQNVKHPVTGQFL (SEQ ID NO: 509) and/or an epitope comprising the sequence LIERIPELNKVARAAAE (SEQ ID NO: 510);
(k) an epitope comprising the sequence LNENSYVPREAGSQKDENL (SEQ ID NO: 511);
(l) an epitope comprising the sequence GTKKDFEDL (SEQ ID NO: 512);
(m) an epitope comprising the sequence SVIIVDKNGRLVYLVENPGGYVAYSK (SEQ ID NO: 513);
(n) an epitope comprising the sequence LLNENSYVPREAGSQKDEN (SEQ ID NO: 514) and/or an epitope comprising the sequence DQTKFPIVNAEL (SEQ ID NO: 515) and/or an epitope comprising the sequence TYKELIERIPELNK (SEQ ID NO: 516);
(o) an epitope comprising the sequence LLNENSYVPREAGSQKDEN (SEQ ID NO: 514) and/or an epitope comprising the sequence TYKELIERIPELNK (SEQ ID NO: 516);
(p) an epitope comprising the sequence SVIIVDKNGRLVYLVENPGGYVAY (SEQ ID NO: 517);
(q) an epitope comprising the sequence IYMDQTKFPIVNAEL (SEQ ID NO: 506) and/or an epitope comprising the sequence FGNMEGDCPSDWKTDSTCRM (SEQ ID NO: 518);
(r) an epitope comprising the sequence LLNENSYVPREAGSQKDENLAL (SEQ ID NO: 505) and/or an epitope comprising the sequence LVENPGGYVAYSKAATVTGKL (SEQ ID NO: 520) and/or an epitope comprising the sequence IYMDQTKFPIVNAELSF (SEQ ID NO: 521) and/or an epitope comprising the sequence ISRAAAEKL (SEQ ID NO: 522) and/or an epitope comprising the sequence VTSESKNVKLTVSNVLKE (SEQ ID NO: 523) and/or an epitope comprising the sequence FCEDTDYPYLGTTMDT (SEQ ID NO: 524);
(s) an epitope comprised within or overlapping with the sequence LLNENSYVPREAGSQKDENLAL (SEQ ID NO: 505) and/or an epitope comprised within or overlapping with the sequence IYMDQTKFPIVNAEL (SEQ ID NO: 506) and/or an epitope comprised within or overlapping with the sequence TYKEL (SEQ ID NO: 507);
(t) an epitope comprised within or overlapping with the sequence KRKLSEKLDSTDFTGTIKL (SEQ ID NO: 508) and/or an epitope comprised within or overlapping with the sequence YTLIEKTMQNVKHPVTGQFL (SEQ ID NO: 509) and/or an epitope comprised within or overlapping with the sequence LIERIPELNKVARAAAE (SEQ ID NO: 510);
(u) an epitope comprised within or overlapping with the sequence LNENSYVPREAGSQKDENL (SEQ ID NO: 511);
(v) an epitope comprised within or overlapping with the sequence GTKKDFEDL (SEQ ID NO: 512);
(w) an epitope comprised within or overlapping with the sequence SVIIVDKNGRLVYLVENPGGYVAYSK (SEQ ID NO: 513);
(x) an epitope comprised within or overlapping with the sequence LLNENSYVPREAGSQKDEN (SEQ ID NO: 514) and/or an epitope comprised within or overlapping with the sequence DQTKFPIVNAEL (SEQ ID NO: 515) and/or an epitope comprised within or overlapping with the sequence TYKELIERIPELNK (SEQ ID NO: 516);
(y) an epitope comprised within or overlapping with the sequence LLNENSYVPREAGSQKDEN (SEQ ID NO: 514) and/or an epitope comprised within or overlapping with the sequence TYKELIERIPELNK (SEQ ID NO: 516);
(z) an epitope comprised within or overlapping with the sequence SVIIVDKNGRLVYLVENPGGYVAY (SEQ ID NO: 517);
(aa) an epitope comprised within or overlapping with the sequence IYMDQTKFPIVNAEL (SEQ ID NO: 506) and/or an epitope comprised within or overlapping with the sequence FGNMEGDCPSDWKTDSTCRM (SEQ ID NO: 518); and
(bb) an epitope comprised within or overlapping with the sequence LLNENSYVPREAGSQKDENLAL (SEQ ID NO: 505) and/or an epitope comprised within or overlapping with the sequence LVENPGGYVAYSKAATVTGKL (SEQ ID NO: 520) and/or an epitope comprised within or overlapping with the sequence IYMDQTKFPIVNAELSF (SEQ ID NO: 521) and/or an epitope comprised within or overlapping with the sequence ISRAAAEKL (SEQ ID NO: 522) and/or an epitope comprised within or overlapping with the sequence VTSESKNVKLTVSNVLKE (SEQ ID NO: 523) and/or an epitope comprised within or overlapping with the sequence FCEDTDYPYLGTTMDT (SEQ ID NO: 524).

18. The antigen-binding protein of claim 17, wherein the antigen binding protein comprises an antibody or antigen-binding fragment thereof which binds to one or more epitopes of hTfR selected from:
(a) an epitope consisting of the sequence LLNE (SEQ ID NO: 525) and/or an epitope consisting of the sequence TYKEL (SEQ ID NO: 507);
(b) an epitope consisting of the sequence DSTDFTGT (SEQ ID NO: 526) and/or an epitope consisting of the sequence VKHPVTGQF (SEQ ID NO: 527) and/or an epitope consisting of the sequence IERIPEL (SEQ ID NO: 528);
(c) an epitope consisting of the sequence LNENSYVPREAGSQKDEN (SEQ ID NO: 529);
(d) an epitope consisting of the sequence FEDL (SEQ ID NO: 519);
(e) an epitope consisting of the sequence IVDKNGRL (SEQ ID NO: 530);
(f) an epitope consisting of the sequence IVDKNGRLVY (SEQ ID NO: 531);
(g) an epitope consisting of the sequence DQTKF (SEQ ID NO: 532);
(h) an epitope consisting of the sequence LVENPGGY (SEQ ID NO: 533) and/or an epitope consisting of the sequence PIVNAELSF (SEQ ID NO: 534) and/or an epitope consisting of the sequence PYLGTTMDT (SEQ ID NO: 535);
(i) an epitope consisting of the sequence LLNENSYVPREAGSQKDENLAL (SEQ ID NO: 505) and/or an epitope consisting of the sequence IYMDQTKFPIVNAEL (SEQ ID NO: 506) and/or an epitope consisting of the sequence TYKEL (SEQ ID NO: 507);
(j) an epitope consisting of the sequence KRKLSEKLDSTDFTGTIKL (SEQ ID NO: 508) and/or an epitope consisting of the sequence YTLIEKTMQNVKHPVTGQFL (SEQ ID NO: 509) and/or an epitope consisting of the sequence LIERIPELNKVARAAAE (SEQ ID NO: 510);
(k) an epitope consisting of the sequence LNENSYVPREAGSQKDENL (SEQ ID NO: 511);
(l) an epitope consisting of the sequence GTKKDFEDL (SEQ ID NO: 512);
(m) an epitope consisting of the sequence SVIIVDKNGRLVYLVENPGGYVAYSK (SEQ ID NO: 513);
(n) an epitope consisting of the sequence LLNENSYVPREAGSQKDEN (SEQ ID NO: 514) and/or an epitope consisting of the sequence DQTKFPIVNAEL (SEQ ID NO: 515) and/or an epitope consisting of the sequence TYKELIERIPELNK (SEQ ID NO: 516);
(o) an epitope consisting of the sequence LLNENSYVPREAGSQKDEN (SEQ ID NO: 514) and/or an epitope consisting of the sequence TYKELIERIPELNK (SEQ ID NO: 516);
(p) an epitope consisting of the sequence SVIIVDKNGRLVYLVENPGGYVAY (SEQ ID NO: 517);
(q) an epitope consisting of the sequence IYMDQTKFPIVNAEL (SEQ ID NO: 506) and/or an epitope consisting of the sequence FGNMEGDCPSDWKTDSTCRM (SEQ ID NO: 518); and
(r) an epitope consisting of the sequence LLNENSYVPREAGSQKDENLAL (SEQ ID NO: 505) and/or an epitope consisting of the sequence LVENPGGYVAYSKAATVTGKL (SEQ ID NO: 520) and/or an epitope consisting of the sequence IYMDQTKFPIVNAELSF (SEQ ID NO: 521) and/or an epitope consisting of the sequence ISRAAAEKL (SEQ ID NO: 522) and/or an epitope consisting of the sequence VTSESKNVKLTVSNVLKE (SEQ ID NO: 523) and/or an epitope consisting of the sequence FCEDTDYPYLGTTMDT (SEQ ID NO: 524).

19. The antigen-binding protein of claim 17 or 18, wherein the antigen-binding protein is selected from a humanized antibody or antigen binding fragment thereof, human antibody or antigen binding fragment thereof, murine antibody or antigen binding fragment thereof, chimeric antibody or antigen binding fragment thereof, monovalent Fab′, divalent Fab2, F(ab)′3 fragments, single-chain fragment variable (scFv), bis-scFv, (scFv)2, diabody, bivalent antibody, one-armed antibody, minibody, nanobody, triabody, tetrabody, disulfide stabilized Fv protein (dsFv), single-domain antibody (sdAb), Ig NAR, single heavy chain antibody, bispecific antibody or biding fragment thereof, bi-specific T-cell engager (BiTE), trispecific antibody, or chemically modified derivatives thereof.

20. A fusion protein comprising the antigen-binding protein of any one of claims 1-19 fused to a payload.

21. A fusion protein comprising an antigen-binding protein that binds specifically to transferrin receptor or an antigenic-fragment thereof or variant thereof fused to a payload, wherein the antigen-binding protein binds to human transferrin receptor with a K_{D} of about 41 nM or a stronger affinity.

22. The fusion protein of claim 20 or 21, wherein the antigen-binding protein binds to human transferrin receptor with a K_{D} of about 3 nM or a stronger affinity, or wherein the antigen-binding protein binds to human transferrin receptor with a K_{D} of about 0.45 nM to 3 nM.

23. The fusion protein of any one of claims 20-22, wherein the payload is one or more antibodies or antigen-binding fragments thereof, proteins, enzymes or viral vectors containing one or more polynucleotides or oligonucleotides; or human alpha-glucosidase polypeptide (hGAA) or a variant thereof.

24. The fusion protein of any one of claims 20-23, wherein the payload is a lysosomal storage disease therapeutic agent (LSD-TA), optionally wherein the payload is an LSD-TA which is Miglustat, Eliglustat, α-galactosidase A; ceramidase; β-glucosidase; saposin-C activator; acid sphingomyelinase; β-galactosidase; β-hexosaminidase A and B; β-hexosaminidase A; GM2-activator protein; GM3 synthase; arylsulfatase A; sphingolipid activator; α-iduronidase; iduronidase-2-sulphatase; heparan N-sulphatase; N-acetyl-α-glucosaminidase; acetyl-CoA; α-glucosamide N-acetyltransferase; N-acetylglucosamine-6-sulphatase; N-acetylgalactosamine-6-sulphate sulphatase; β-galactosidase; N-acetylgalactosamine-4-sulphatase (arylsulphatase B); β-glucuronidase; hylauronidase; α-hlucosidase 2; or lysosomal acid lipase; or
a polypeptide or a polypeptide encoded by a human gene specified in any one of Tables C-N or a variant thereof.

25. A fusion protein comprising an antigen-binding protein that binds specifically to human transferrin receptor, which comprises a heavy chain variable region (HCVR or V_{H}) and a light chain variable region (LCVR or V_{L}),
which is fused to an alpha-glucosidase polypeptide (GAA),
wherein a Fab having said V_{H} and V_{L} binds to human transferrin receptor with a K_{D} of about 0.65 nM or a greater affinity; and
wherein, when said fusion protein is administered to a mouse expressing human transferrin receptor in the brain, the mouse achieves a molar ratio of mature GAA protein in the brain:serum GAA protein, in the mouse, of about 1:1 or greater when normalized against said ratio in mouse expressing mouse transferrin receptor that was administered 8D3.

26. The fusion protein of claim 25, wherein the antigen-binding protein is a Fab or a single chain fragment variable (scFv).

27. The fusion protein of claim 26, wherein the fusion protein comprises the amino acid sequence set forth in SEQ ID NO: 408 (or a variant thereof) or comprises the amino acid sequence set forth in SEQ ID NO: 405 (or a variant thereof).

28. The fusion protein of claim 26 or 27, wherein the fusion protein comprises the amino acid sequence set forth in SEQ ID NO: 408 (or a variant thereof).

29. The fusion protein of claim 25, wherein the antigen-binding protein is an antibody or antigen-binding fragment thereof.

30. The fusion protein of any one of claims 20-28, wherein the antigen-binding protein is an scFv comprising domains arranged in the following orientation: N-Heavy chain variable region-Light chain variable region-GAA protein-C.

31. The fusion protein of any one of claims 20-28, wherein the antigen-binding protein is an scFv comprising domains arranged in the following orientation: N-Light chain variable region-Heavy chain variable region-GAA protein-C.

32. The fusion protein of any one of claims 24-31, wherein the antigen-binding protein is an scFv, wherein said scFv and GAA are connected by a peptide linker.

33. The fusion protein of claim 32, wherein the scFv and GAA are connected by a peptide linker which is -(GGGGS)ₘ- (SEQ ID NO: 426); wherein m is 1, 2, 3, 4, 5, 6, 7, 8, 9 or 10.

34. The fusion protein of any one of claims 24-33, wherein the antigen-binding protein is an scFv and said scFv variable regions are connected by a peptide linker.

35. The fusion protein of claim 34, wherein the scFv variable regions are connected by a peptide linker which is -(GGGGS)ₙ- (SEQ ID NO: 426); wherein n is 1, 2, 3, 4, 5, 6, 7, 8, 9 or 10.

36. The fusion protein of any one of claims 24-35, wherein the fusion protein binds to human transferrin receptor with a K_{D} of about 1×10⁻⁷ M or a greater affinity.

37. The fusion protein of any one of claims 24-36 which comprises:
(i) a HCVR that comprises the HCDR1, HCDR2 and HCDR3 of a HCVR comprising the amino acid sequence set forth in SEQ ID NO: 2; 462; 12; 463; 22; 464; 32; 42; 52; 467; 62; 492; 72; 470; 82; 92; 472; 102; 112; 473; 122; 132; 142; 475; 152; 162; 477; 172; 182; 478; 192; 480; 202; 481; 212; 222; 232; 242; 252; 482; 262; 272; 282; 292; 302; 483 or 312 (or a variant thereof); and/or
(ii) a LCVR that comprises the LCDR1, LCDR2 and LCDR3 of a LCVR comprising the amino acid sequence set forth in SEQ ID NO: 7; 17; 27; 37; 465; 47; 466; 57; 468; 67; 469; 77; 471; 87; 97; 107; 117; 474; 127; 137; 147; 476; 157; 167; 177; 187; 479; 197; 207; 217; 227; 237; 247; 257; 267; 277; 287; 297; 307; 488; 317 or 484 (or a variant thereof).

38. The fusion protein of any one of claims 24-37 which is an scFv that comprises a heavy chain variable region (V_{H}) and a light chain variable region (V_{L}), and an alpha-glucosidase polypeptide (GAA), wherein said V_{H}, V_{L} and GAA are arranged as follows:
(i) V_{L}-V_{H}-GAA;
(ii) V_{H}-V_{L}-GAA;
(iii) V_{L}-[(GGGGS)₃ (SEQ ID NO: 538)]-V_{H}-[(GGGGS)₂ (SEQ ID NO: 537)]-GAA; or
(iv) V_{H}-[(GGGGS)₃ (SEQ ID NO: 538)]-V_{L}-[(GGGGS)₂ (SEQ ID NO: 537)]-GAA.

39. The fusion protein of any one of claims 24-38 comprising: the amino acid sequence set forth in a member selected from the group consisting of SEQ ID NOs: 392-423; SEQ ID NO: 321 (optionally lacking the N-terminal MHRPRRRGTRPPPLALLAALLLAARGADA (SEQ ID NO: 500) signal sequence); SEQ ID NO: 322 (optionally lacking the N-terminal MHRPRRRGTRPPPLALLAALLLAARGADA (SEQ ID NO: 500) signal sequence); SEQ ID NO: 323 (optionally lacking the N-terminal MHRPRRRGTRPPPLALLAALLLAARGADA (SEQ ID NO: 500) signal sequence); and SEQ ID NO: 324 (optionally lacking the N-terminal MIRPRRRGTRPPPLALLAALLLAARGADA (SEQ ID NO: 500) signal sequence); or a variant thereof.

40. The fusion protein of any one of claims 24-39, wherein the antigen-binding protein, which when not fused to a GAA polypeptide, does not block more than 50% of binding of a human transferrin receptor C-terminal fragment to human holo-transferrin that occurs in the absence of such single chain fragment variable (scFv), antibody or an antigen-binding fragment.

41. The fusion protein of claim 40, wherein said blocking is as measured in an Enzyme Linked Immunosorbent Assay (ELISA) plate assay wherein binding of human transferrin receptor extracellular domain that is fused to a His6-myc-myc tag is pre-bound to said scFv, antibody or antigen-binding fragment and then contacted with holo-transferrin which is immobilized to the surface of the plate by binding of an anti-holo-transferrin antibody that is bound to the plate.

42. The fusion protein of claim 40 or 41, wherein binding of the holo-transferrin and human transferrin receptor extracellular domain in the absence of the scFv, antibody or antigen-binding fragment is measured at a concentration of about 300 pM human transferrin receptor extracellular domain.

43. A fusion protein or antigen-binding protein that binds specifically to human transferrin receptor which has one or more of the following characteristics:
affinity (K_{D}) for binding to human TfR at 25° C. in surface plasmon resonance format of about 41 nM or a higher affinity;
affinity (K_{D}) for binding to monkey TfR at 25° C. in surface plasmon resonance format of about 0 nM (no detectable binding) or a higher affinity;
ratio of [K_{D} for binding to monkey TfR/K_{D} for binding to human TfR] at 25° C. in surface plasmon resonance format of from 0 to 278;
blocks about 3-13% hTfR binding to Human Holo-Tf when in Fab format (IgG1);
blocks about 6-13% hTfR binding to Human Holo-Tf when in scFv (V_{K}-V_{H}) format;
blocks about 11-26% hTfR binding to Human Holo-Tf when in scFv (V_{H}-V_{L}) format;
when in anti-hTfR scFv:hGAA format, exhibits a ratio of about 1-2 mature hGAA protein in brain (normalized to that of positive control 8D3:GAA scFv) when administered to mice expressing human transferrin receptor (optionally, lacking functional endogenous GAA);
when in anti-hTfR scFv:hGAA format, exhibits a ratio of about 0.1-1.2 mature hGAA protein in brain parenchyma (normalized to that of positive control 8D3:GAA scFv) when administered to mice expressing human transferrin receptor (optionally, lacking functional endogenous GAA);
when in anti-hTfR scFv:hGAA format, exhibits a ratio of about 0.67, 1.80, 1.78 or 7.74 (about 1-2) mature hGAA protein in quadriceps (normalized to that of positive control 8D3:GAA scFv) when administered to mice expressing human transferrin receptor (optionally, lacking functional endogenous GAA);
when in anti-hTfR scFv:hGAA format, exhibits a ratio of about 0.1-1.2 mature hGAA protein in brain parenchyma (normalized to that of positive control 8D3:GAA scFv) when administered to mice expressing human transferrin receptor (optionally, lacking functional endogenous GAA);
when in anti-hTfR scFv:hGAA format, delivers mature hGAA protein to serum, liver, cerebrum, cerebellum, spinal cord, heart and/or quadricep when administered to mice expressing human transferrin receptor (optionally, lacking functional endogenous GAA);
when in anti-hTfR scFv:hGAA format, reduces glycogen stored in cerebrum, cerebellum, spinal cord, heart and/or quadricep when administered to mice expressing human transferrin receptor (optionally, lacking functional endogenous GAA);
when comprising the antigen-binding protein fused to GAA, reduces glycogen levels in cerebellum of mice expressing human transferrin receptor but lacking functional endogenous GAA by at least about 90% relative to that of untreated mice;
when comprising the antigen-binding protein fused to GAA, reduces glycogen levels in quadricep of mice expressing human transferrin receptor but lacking functional endogenous GAA by at least about 89% relative to that of untreated mice; and/or
does not cause abnormal iron homeostasis when administered to mice expressing human transferrin receptor.

44. A pharmaceutical composition comprising the fusion protein of any one of claims 20-43 or the antigen-binding protein of any one of claims 1-19 and 43 and a pharmaceutically acceptable carrier.

45. A composition or kit comprising the fusion protein of any one of claims 20-43 or the antigen-binding protein of any one of claims 1-19 and 43 or a pharmaceutical composition thereof in association with a further therapeutic agent.

46. The composition or kit of claim 45, wherein the further therapeutic agent is selected from: alglucosidase alfa, rituximab, methotrexate, intravenous immunoglobulin (IVIG), avalglucosidase alfa, levalbuterol, an antibiotic, cortisone, prednisone, a bisphosphonate, and palivizumab.

47. The composition or kit of claim 45, wherein the further therapeutic agent is selected from: a beta2-adrenergic agonist, a steroid, a bisphosphonate, an infectious disease treatment, a vaccine, and a Pneumococcal vaccine.

48. A complex comprising the fusion protein of any one of claims 20-43 or the antigen-binding protein of any one of claims 1-19 and 43 bound to a human transferrin receptor polypeptide or antigenic fragment thereof.

49. An isolated polynucleotide encoding the fusion protein of any one of claims 20-43 or the antigen-binding protein of any one of claims 1-19 and 43.

50. The polynucleotide of claim 49 that comprises the nucleotide sequence set forth in SEQ ID NO: 1; 6; 11; 16; 21; 26; 31; 36; 41; 46; 51; 56; 61; 66; 71; 76; 81; 86; 91; 96; 101; 106; 111; 116; 121; 126; 131; 136; 141; 146; 151; 156; 161; 166; 171; 176; 181; 186; 191; 196; 201; 206; 211; 216; 221; 226; 231; 236; 241; 246; 251; 256; 261; 266; 271; 276; 281; 286; 291; 296; 301; 306; 311; and/or 316.

51. The polynucleotide of claim 49 or 50 that comprises:
(1) the nucleotide sequence set forth in SEQ ID NO: 1 and SEQ ID NO: 6;
(2) the nucleotide sequence set forth in SEQ ID NO: 11 and SEQ ID NO: 16;
(3) the nucleotide sequence set forth in SEQ ID NO: 21 and SEQ ID NO: 26;
(4) the nucleotide sequence set forth in SEQ ID NO: 31 and SEQ ID NO: 36;
(5) the nucleotide sequence set forth in SEQ ID NO: 41 and SEQ ID NO: 46;
(6) the nucleotide sequence set forth in SEQ ID NO: 51 and SEQ ID NO: 56;
(7) the nucleotide sequence set forth in SEQ ID NO: 61 and SEQ ID NO: 66;
(8) the nucleotide sequence set forth in SEQ ID NO: 71 and SEQ ID NO: 76;
(9) the nucleotide sequence set forth in SEQ ID NO: 81 and SEQ ID NO: 86;
(10) the nucleotide sequence set forth in SEQ ID NO: 91 and SEQ ID NO: 96;
(11) the nucleotide sequence set forth in SEQ ID NO: 101 and SEQ ID NO: 106;
(12) the nucleotide sequence set forth in SEQ ID NO: 111 and SEQ ID NO: 116;
(13) the nucleotide sequence set forth in SEQ ID NO: 121 and SEQ ID NO: 126;
(14) the nucleotide sequence set forth in SEQ ID NO: 131 and SEQ ID NO: 136;
(15) the nucleotide sequence set forth in SEQ ID NO: 141 and SEQ ID NO: 146;
(16) the nucleotide sequence set forth in SEQ ID NO: 151 and SEQ ID NO: 156;
(17) the nucleotide sequence set forth in SEQ ID NO: 161 and SEQ ID NO: 166;
(18) the nucleotide sequence set forth in SEQ ID NO: 171 and SEQ ID NO: 176;
(19) the nucleotide sequence set forth in SEQ ID NO: 181 and SEQ ID NO: 186;
(20) the nucleotide sequence set forth in SEQ ID NO: 191 and SEQ ID NO: 196;
(21) the nucleotide sequence set forth in SEQ ID NO: 201 and SEQ ID NO: 206;
(22) the nucleotide sequence set forth in SEQ ID NO: 211 and SEQ ID NO: 216;
(23) the nucleotide sequence set forth in SEQ ID NO: 221 and SEQ ID NO: 226;
(24) the nucleotide sequence set forth in SEQ ID NO: 231 and SEQ ID NO: 236;
(25) the nucleotide sequence set forth in SEQ ID NO: 241 and SEQ ID NO: 246;
(26) the nucleotide sequence set forth in SEQ ID NO: 251 and SEQ ID NO: 256;
(27) the nucleotide sequence set forth in SEQ ID NO: 261 and SEQ ID NO: 266;
(28) the nucleotide sequence set forth in SEQ ID NO: 271 and SEQ ID NO: 276;
(29) the nucleotide sequence set forth in SEQ ID NO: 281 and SEQ ID NO: 286;
(30) the nucleotide sequence set forth in SEQ ID NO: 291 and SEQ ID NO: 296;
(31) the nucleotide sequence set forth in SEQ ID NO: 301 and SEQ ID NO: 306; and/or
(32) the nucleotide sequence set forth in SEQ ID NO: 311 and SEQ ID NO: 316.

52. A vector comprising the polynucleotide one any one of claims 49-51.

53. A host cell comprising the fusion protein of any one of claims 20-43, the antigen-binding protein of any one of claims 1-19 and 43, the polynucleotide of any one of claims 49-51, or the vector of claim 52.

54. The host cell of claim 53 which is a Chinese hamster ovary (CHO) cell.

55. A method for making the fusion protein of any one of claims 20-43 or the antigen-binding protein of any one of claims 1-19 and 43, comprising culturing a host cell comprising a polynucleotide that encodes the fusion protein or antigen-binding protein in a culture medium under conditions favorable to expression of the fusion protein or antigen-binding protein.

56. The method of claim 55 comprising the steps:
(a) introducing said polynucleotide into a host cell;
(b) culturing the host cell under conditions favorable to expression of the fusion protein or antigen-binding protein; and
(c) optionally, isolating the fusion protein or antigen-binding protein from the culture medium and/or host cell; and
(d) optionally, chemically conjugating the antigen-binding protein to a payload.

57. A fusion protein or antigen-binding protein which is the product of a method of claim 55 or 56.

58. A vessel or injection device comprising the fusion protein of any one of claims 20-43 and 57 or the antigen-binding protein of any one of claims 1-19, 43, and 57.

59. A method for administering the fusion protein of any one of claims 20-43 and 57 or the antigen-binding protein of any one of claims 1-19, 43, and 57 to a subject comprising introducing the protein into the body of the subject.

60. The method of claim 59, wherein said fusion protein or antigen-binding protein is introduced into the body of the subject parenterally.

61. A method for treating or preventing a lysosomal storage disease in a subject in need thereof comprising administering, to the subject, an effective amount of the fusion protein of any one of claims 20-43 and 57, wherein the payload is a lysosomal storage disease therapeutic agent (LSD-TA).

62. The method of claim 61, wherein the lysosomal storage disease is: Fabry disease; Farber lipogranulomatosis; Gaucher disease type I; Gaucher disease (type II or III); Niemann-Pick diseases (type A or B); GM1-gangliosidosis; GM2-gangliosidosis (Sandhoff); GM2-gangliosidosis (Tay-Sachs); GM2-gangliosidosis (GM2-activator deficiency); GM3-gangliosidosis; Metachromatic leukodystrophy; Sphingolipid-activator deficiency; MPS I (Scheie, Hurler-Scheie, or Hurler disease); MPS II (Hunter); MPS IIIA (Sanfilippo A); MPS IIIB (Sanfilippo B); MPS IIIC (Sanfilippo C); MPS IIID (Sanfilippo D); MPS IVA (Morquio syndrome A); MPS IVB (Morquio syndrome B); MPS VI (Maroteaux-Lamy); MPS VII (Sly disease); MPS IX; Pompe (glycogen storage disease type II); or Lysosomal acid lipase deficiency (LAL-D; Wolman disease).

63. The method of claim 61 or 62, wherein one or more signs or symptoms of the LSD in the subject are alleviated after the fusion protein or antigen-binding protein is administered.

64. A method for treating or preventing a glycogen storage disease (GSD)) in a subject in need thereof comprising administering, to the subject, an effective amount of the fusion protein of any one of claims 20-43 and 57.

65. The method of claim 64, wherein the glycogen storage disease is Pompe disease.

66. The method of claim 65, wherein the Pompe disease is classic infantile-onset form Pompe disease.

67. The method of claim 65, wherein the Pompe disease is non-classic infantile form Pompe disease.

68. The method of claim 65, wherein the Pompe disease is late onset form Pompe disease.

69. The method of any one of claims 64-68, wherein the subject has a GAA genotype selected from the group consisting of:
ASP91ASN; MET318THR; GLU521LYS; GLY643ARG; ARG725TRP; IVS1AS, T-G, −13; LYS903DEL; LEU299ARG; SER529VAL; ASP645GLU; GLU689LYS; EX18DEL; PRO545LEU; 1-BP DEL, 525T; ARG854TER; ALA237VAL; GLY293ARG; and IVS6AS, G-C, −1.

70. The method of any one of claims 64-69, wherein the subject is administered the fusion protein in association with a further therapeutic agent.

71. The method of claim 70, wherein the further therapeutic agent is selected from: alglucosidase alfa, rituximab, methotrexate, intravenous immunoglobulin (IVIG), avalglucosidase alfa, levalbuterol, an antibiotic, cortisone, prednisone, a bisphosphonate, and palivizumab.

72. The method of claim 70, wherein the further therapeutic agent is selected from: a beta2-adrenergic agonist, a steroid, a bisphosphonate, an infectious disease treatment, a vaccine, and a pneumococcal vaccine.

73. The method of any one of claims 64-72, wherein the subject is 1 year of age or less and experiences a symptom selected from:
trouble eating and not gaining weight;
poor head and neck control;
rolling over and sitting up later than expected;
breathing problems;
lung infection;
enlarged and thickening heart
heart defect;
enlarged liver; and
enlarged tongue.

74. The method of any one of claims 64-73, wherein the subject is an adult and experiences a symptom selected from:
weakness in the legs, trunk, and/or arms;
shortness of breath;
lung infection;
trouble breathing while sleeping;
spine curvature;
enlarged liver;
enlarged tongue; and
stiff joints.

75. The method of any one of claims 64-74, wherein one or more signs or symptoms of the GSD in the subject are alleviated after the fusion protein or antigen-binding protein is administered.

76. A method for delivering a payload to a tissue or cell type in the body of a subject comprising administering, to the subject, an antigen-binding protein that binds specifically to transferrin receptor or an antigenic-fragment thereof or variant thereof to the subject fused to the payload.

77. The method of claim 76, wherein the payload is one or more antibodies or antigen-binding fragments thereof, proteins, enzymes or viral vectors containing one or more polynucleotides or oligonucleotides.

78. The method of claim 76 or 77, wherein the payload is human GAA protein or a variant thereof.

79. The method of any one of claims 76-78, wherein the tissue is brain/spinal cord/CNS; eye; skeletal muscle; adipose tissue; blood/bone marrow; breast; lung/bronchus; colon; uterus; esophagus; heart; kidney; liver; lymph node; ovary; pancreas; placenta; prostate; rectum; skin; peripheral blood mononuclear cell (PBMC); small intestine; spleen; stomach; testis; peripheral nervous system; and/or bone/cartilage/joint.

80. The method of any one of claims 76-79, wherein the cell type and tissue that is associate with the cell type is as follows:

| | |
|---|---|
| (1) brain/spinal cord/CNS tissue | endothelial cells |
| | neurons (all types) |
| | oligodendrocytes (and/or precursors) |
| | pericytes |
| | meninges/leptomeningeal cells |
| | arachnoid barrier cells |
| | peripheral glia |
| | astrocytes |
| | glia |
| | Schwann cells |
| | ependymal cells |
| | microglia; |
| (2) eye tissue | rod photoreceptor cells |
| | Muller glia cells |
| | bipolar cells |
| | cone photoreceptor cells |
| | endothelial cells |
| | cornea |
| | sclera |
| | optic nerve |
| | pupillary sphincter; |
| (3) skeletal muscle tissue | skeletal myocytes |
| | fibroblasts |
| | endothelial cells |
| | macrophages |
| | satellite cells; |
| (4) adipose tissue | adipocytes |
| | fibroblasts |
| | T-cells |
| | macrophages |
| | B-cells |
| | dendritic cells; |
| (5) blood/bone marrow tissue | T-cells |
| | B-cells |
| | macrophages |
| | erythroid cells |
| | plasmid cells |
| | dendritic cells; |
| (6) breast tissue | glandular cells |
| | T-cells |
| | fibroblasts |
| | macrophages |
| | endothelial cells |
| | myoepithelial cells |
| | adipocytes; |
| (7) lung/bronchus tissue | basal respiratory cells |
| | respiratory ciliated cells |
| | club cells |
| | smooth muscle cells |
| | ionocytes |
| | macrophages |
| | alveolar cells (type 1 and/or 2) |
| | T-cells |
| | endothelial cells; |
| (8) colon tissue | distal enterocytes |
| | intestinal goblet cells |
| | undifferentiated cells |
| | T-cells |
| | Paneth cells |
| | B-cells |
| | enteroendocrine cells; |
| (9) uterus tissue | glandular and luminal cells |
| | endometrial stromal cells |
| | endothelial cells |
| | smooth muscle cells |
| | T-cells |
| | macrophages; |
| (10) esophagus tissue | fibroblasts |
| | squamous epithelial cells |
| | endothelial cells |
| | smooth muscle cells |
| | macrophages |
| | plasma cells |
| | T-cells; |
| (11) heart tissue | cardiomyocytes |
| | endothelial cells |
| | fibroblasts |
| | macrophages |
| | T-cells |
| | B-cells |
| | dendritic cells; |
| (12) kidney tissue | proximal tubular cells |
| | T-cells |
| | macrophages |
| | collecting duct cells |
| | B-cells |
| | glomeruli |
| | fibroblasts; |
| (13) liver tissue | hepatocytes |
| | B-cells |
| | erythroid cells; |
| (14) lymph node tissue | B-cells |
| | T-cells; |
| (15) ovary tissue | granulosa cells |
| | fibroblasts |
| | smooth muscle cells |
| | macrophages |
| | T-cells |
| | theca cells |
| | fibroblasts; |
| (16) pancreas tissue | ductal cells |
| | pancreatic endocrine cells |
| | smooth muscle cells |
| | endothelial cells |
| | macrophages |
| | exocrine glandular cells |
| | monocytes; |
| (17) placenta tissue | cytotrophoblasts |
| | extravillous trophoblasts |
| | fibroblasts |
| | Hofbauer cells |
| | endothelial cells; |
| (18) prostate tissue | basal prostatic cells |
| | prostatic glandular cells |
| | urothelial cells |
| | endothelial cells |
| | fibroblasts |
| | smooth muscle cells |
| | macrophages |
| | T-cells; |
| (19) rectum tissue | undifferentiated cells |
| | intestinal goblet cells |
| | Paneth cells |
| | distal enterocytes |
| | enteroendocrine cells; |
| (20) skin tissue | Langerhans cells |
| | fibroblasts |
| | endothelial cells |
| | basal keratinocytes |
| | suprabasal keratinocytes |
| | T-cells |
| | smooth muscle cells |
| | melanocytes; |
| (21) PBMC tissue | monocytes |
| | T-cells |
| | NK-cells |
| | dendritic cells; |
| (22) small intestine tissue | proximal enterocytes |
| | undifferentiated cells |
| | intestinal goblet cells |
| | Paneth cells; |
| (23) spleen tissue | B-cells |
| | T-cells |
| | plasma cells |
| | macrophages; |
| (24) stomach tissue | B-cells |
| | T-cells |
| | gastric mucus-secreting cells |
| | plasma cells |
| | fibroblasts |
| | macrophages; |
| (25) testes tissue | Leydig cells |
| | late spermatids |
| | spermatogonia |
| | early spermatids |
| | macrophages |
| | spermatocytes |
| | peritubular cells |
| | Sertoli cells |
| | endothelial cells; |
| (26) peripheral nervous system | motor neurons |
| tissue | sensory neurons |
| | Schwann cells |
| | dorsal root ganglion; |
| (27) bone/cartilage/joint tissue | chondrocytes |
| | chondroblasts |
| | mesenchymal cells |
| | osteoblasts |
| | osteoclasts. |

81. The method of any one of claims 76-80 which comprises piercing the body of the subject with a needle of a syringe and injecting the antigen-binding protein that binds specifically to transferrin receptor or an antigenic-fragment thereof or variant thereof to the subject fused to the payload into the body of the subject.

82. The method of any one of claims 76-81, wherein the subject suffers from a muscle atrophy condition, metabolic disease, sarcopenia or cachexia.

83. A method of expressing in a cell a fusion protein comprising an antigen-binding protein that binds specifically to transferrin receptor or an antigenic-fragment thereof or variant thereof fused to a payload comprising:
(a) administering to the cell a gene therapy vector comprising the isolated polynucleotide of any one of claims 49-51, wherein the isolated polynucleotide encodes the fusion protein;
(b) allowing the isolated polynucleotide to integrate into a genomic locus of the cell; and
(c) allowing the cell to produce the fusion protein.

84. The method of claim 83 further comprising administering a nuclease agent or one or more polynucleotides encoding the nuclease agent to the cell, wherein the nuclease agent cleaves a nuclease target site in the genomic locus, and the isolated polynucleotide is integrated into the genomic locus.

85. The method of claim 84, wherein the nuclease agent comprises a Clustered Regularly Interspersed Short Palindromic Repeats (CRISPR)/CRISPR-associated (Cas) system, a zinc finger nuclease (ZFN), or a Transcription Activator-Like Effector Nuclease (TALEN).

86. The method of any one of claims 83-85, wherein the cell is in vivo in a subject.

87. The method of any one of claims 83-85, wherein the cell is ex vivo.

88. The method of any one of claims 83-87, wherein the gene therapy vector is a viral vector, a naked polynucleotide, or a polynucleotide complex, optionally wherein the polynucleotide complex is a lipid nanoparticle comprising the polynucleotide.

89. The method of any one of claims 83-88, wherein the gene therapy vector is a viral vector selected from the group consisting of a retrovirus, an adenovirus, a herpes simplex virus, a pox virus, a vaccinia virus, a lentivirus, or an adeno-associated virus.

90. The method of any one of claims 83-89, wherein the gene therapy vector is an adeno-associated virus (AAV) vector, optionally wherein the gene therapy vector is an AAV2/8 chimera and/or an AAV pseudotyped to the liver.

91. The method of any one of claims 83-90, wherein the genomic locus is a safe harbor locus.

92. The method of claim 91, wherein the genomic locus is at or proximal to a locus selected from the group consisting of an EESYR locus, a SARS locus, position 188,083,272 of human chromosome 1 or its non-human mammalian orthologue, position 3,046,320 of human chromosome 10 or its non-human mammalian orthologue, position 67,328,980 of human chromosome 17 or its non-human mammalian orthologue, an adeno-associated virus site 1 (AAVS1) on chromosome, a naturally occurring site of integration of AAV virus on human chromosome 19 or its non-human mammalian orthologue, a chemokine receptor 5 (CCR5) gene, a chemokine receptor gene encoding an HIV-1 coreceptor, a mouse Rosa26 locus or its non-murine mammalian orthologue, and an albumin (alb) locus.

93. The method of any one of claims 83-92, wherein the cell is a human cell.

94. The method of any one of claims 83-93, wherein the cell is a liver cell.
